(19) United States
(12) Patent Application Publication
(10) Pub. No.: US 2025/0221998 A1
(43) Pub. Date: Jul. 10, 2025

(54) DISCOVERY OF COVALENT EGFR INHIBITOR THROUGH CYSTEINE 775

(71) Applicants: Dana-Farber Cancer Institute, Inc., Boston, MA (US); The Brd. of Trust. of the Leland Stan. Jr. Univ., Stanford, CA (US)

(72) Inventors: Nathanael S. Gray, Stanford, CA (US); Tinghu Zhang, Brookline, MA (US); Zhengnian Li, Stanford, CA (US); Jianwei Che, Sharon, MA (US); Stephen Leycester Gwaltney, II, Stamford, CT (US); Dimitrios Gazgalis, Boston, MA (US)

(21) Appl. No.: 18/853,712

(22) PCT Filed: Apr. 5, 2023
(86) PCT No.: PCT/US2023/017602

§ 371 Date: Oct. 2, 2024

Related U.S. Application Data
(60) Provisional application No. 63/362,490, filed on Apr. 5, 2022.

(51) Int. Cl. A61K 31/519; A61P 35/00; C07D 471/04

(52) U.S. Cl. CPC A61K 31/519; A61P 35/00; C07D 471/04

(57) ABSTRACT

The disclosure relates to compounds that act as inhibitors of epidermal growth factor receptor (EGFR); pharmaceutical compositions comprising the compounds; and methods of treating or preventing kinase-mediated disorders, including cancer and other proliferation diseases.

### RELATED APPLICATIONS

This application claims priority to U.S. Provisional Application No. 63/362,490 filed on Apr. 5, 2022, the entire content of which is hereby incorporated by reference in its entirety.

### BACKGROUND

The epidermal growth factor receptor (EGFR, Erb-B1) belongs to a family of receptor tyrosine kinases that mediate the proliferation, differentiation, and survival of normal and malignant cells (Arteaga, C. L., J. Clin. Oncol. 19, 2001, 32-40). Deregulation of EGFR has been implicated in many types of human cancer, with overexpression of the receptor present in at least 70% of human cancers (Seymour, L. K., Curr. Drug Targets 2, 2001, 117-133), including non-small lung cell carcinomas, breast cancers, gliomas, squamous cell carcinomas of the head and neck, and prostate cancer (Raymond, E., et al., Drugs 60 (Suppl. 1), 2000, 15-23, discussion 41-2; Salomon, D. S., et al., Crit. Rev. Oncol. Hematol. 19, 1995, 183-232; Voldborg B. R., et al., Ann. Oncol. 8, 1997, 1197-1206). EGFR has therefore emerged as an attractive target for the design and development of diagnostic and therapeutic agents that can specifically bind and inhibit the receptor's tyrosine kinase activity and signal transduction pathway in cancer cells. For example, the EGFR tyrosine kinase (EGFR-TK) reversible inhibitor TARCEVA® is approved by the FDA for treatment of NSCLC and advanced pancreatic cancer. Other anti-EGFR targeted molecules have also been approved, including Lapatinib and IRESSA®.

The epidermal growth factor receptor (EGFR) is one of the most investigated receptor protein tyrosine kinases and its link to non-small-cell lung cancer (NSCLC) is well established (A. Russo, T. et al., Oncotarget 2015, 6, 26814). However, over 75% of patients die five years after their NSCLC diagnosis. Tumors driven by activating mutations within the EGFR tyrosine kinase domain, e.g., point-mutation L858R or in-frame exon-19 deletions (ex19del) are initially sensitive to EGFR tyrosine kinase inhibitors (TKIs), such as gefitinib and erlotinib, (Paez, J. G., et al., Science (New York, N.Y.) 2004, 304, 1497; Lynch, T. J., et al., The New England Journal of Medicine 2004, 350, 2129), but these inhibitors are rendered resistant due to the acquisition of the secondary ‘gatekeeper’ T790M mutation (Pao, W., et al., PLoS Medicine 2005, 2, e73; Yu, H. A., et al., Clinical Cancer Research 2013, 19, 2240). Efforts to overcome first-generation TKI drug resistance resulted in the discovery and optimization of T790M-targeting irreversible inhibitors, which are rendered effective due to the ability to form covalent bonds with C797 (D. A. E. Cross, et al., Cancer Discovery 2014, 4, 1046; E. L. Kwak, et al., Proceedings of the National Academy of Sciences of the United States of America. 2005, 102, 7665). Patients harboring T790M positive tumors respond well to treatment with AZD9291, and more recently this drug has been shown to be a superior treatment as a front-line therapy in untreated EGFR mutant NSCLC patients (J.-C. Soria, et al., The New England Journal of Medicine 2018, 378, 113). However, despite these successes, patients can acquire resistance to AZD9291 through the acquisition of the C797S mutation that precludes the ability for the drug to form their essential covalent bonds (K. S. Thress, et al., Nature Medicine 2015, 21, 560).

Thus, there is a need for potent small molecule EGFR inhibitors with alternative mechanisms of action targeting mutant EGFR.

### SUMMARY

In an aspect, provided herein is a compound of Formula A-I:

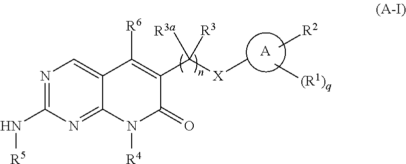

or a pharmaceutically acceptable salt thereof, wherein the variables are defined herein.

In an embodiment, the compound of Formula A-I is a compound of Formula I:

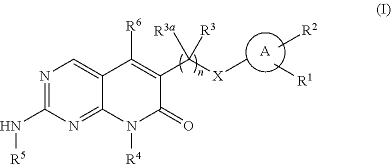

or a pharmaceutically acceptable salt thereof, wherein the variables are defined herein.

In another embodiment, the compound of Formula I is a compound of Formula IA:

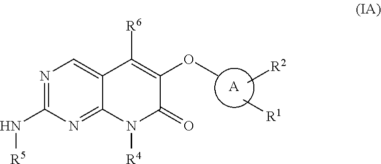

or a pharmaceutically acceptable salt thereof, wherein the variables are defined herein.

In yet another embodiment, the compound of Formula I is a compound of Formula IB:

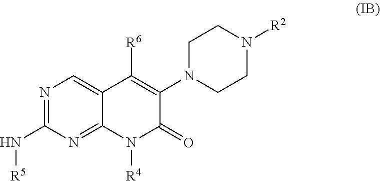

or a pharmaceutically acceptable salt thereof, wherein the variables are defined herein.

In still another embodiment, the compound of Formula I is a compound of Formula IC:

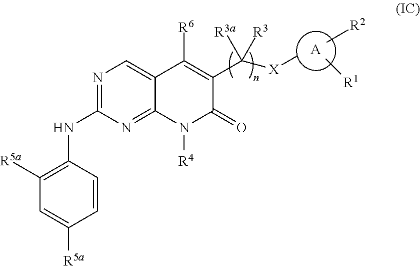

or a pharmaceutically acceptable salt thereof, wherein the variables are defined herein.

In an embodiment, the compound of Formula I is a compound of Formula ID:

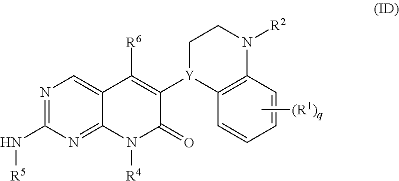

or a pharmaceutically acceptable salt thereof, wherein the variables are defined herein. In another embodiment, the compound of Formula ID is a compound of Formula IDa:

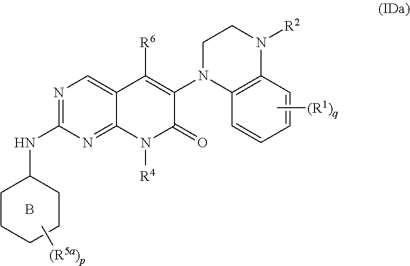

or a pharmaceutically acceptable salt thereof, wherein the variables are defined herein.

In yet another embodiment, the compound of Formula ID is a compound of Formula IDb:

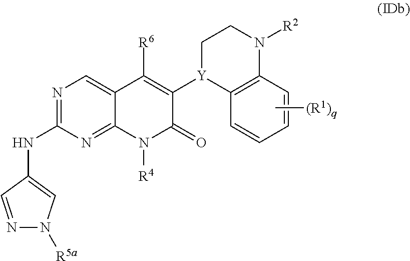

or a pharmaceutically acceptable salt thereof, wherein the variables are defined herein.

In an embodiment, the compound of Formula I is a compound of Formula IE:

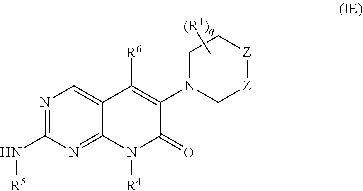

or a pharmaceutically acceptable salt thereof, wherein the variables are defined herein. In another embodiment, the compound of Formula IE is a compound of Formula IEa:

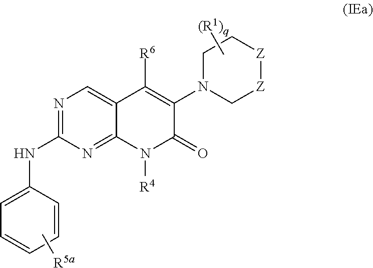

or a pharmaceutically acceptable salt thereof, wherein the variables are defined herein.

In yet another embodiment, the compound of Formula IE is a compound of Formula IEb:

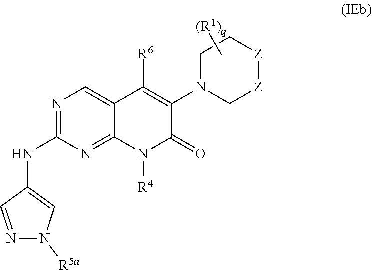

or a pharmaceutically acceptable salt thereof, wherein the variables are defined herein.

In still another embodiment, the compound of Formula I is a compound of Formula IF:

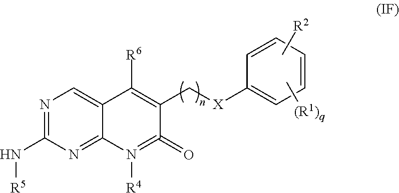

or a pharmaceutically acceptable salt thereof, wherein the variables are defined herein.

In an embodiment, the compound of Formula IF is a compound of Formula IFa:

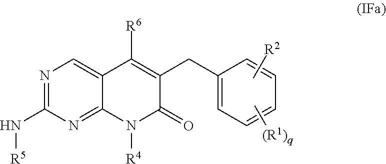

or a pharmaceutically acceptable salt thereof, wherein the variables are defined herein. In another embodiment, the compound of Formula IFa is a compound of Formula IFaa:

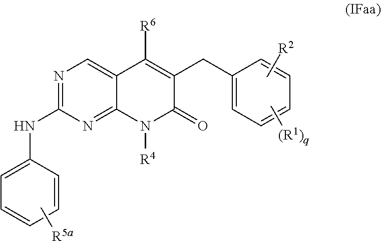

or a pharmaceutically acceptable salt thereof, wherein the variables are defined herein.

In yet another embodiment, the compound of Formula IF is a compound of Formula IFb:

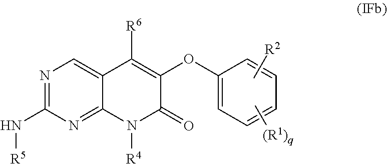

or a pharmaceutically acceptable salt thereof, wherein the variables are defined herein.

In yet another embodiment, the compound of Formula IFb is a compound of Formula IFba:

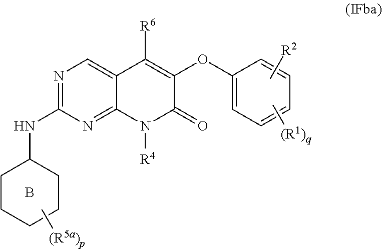

or a pharmaceutically acceptable salt thereof, wherein the variables are defined herein.

In another aspect, provided herein is a method of treating cancer or a proliferation disease, comprising administering to a subject in need thereof an effective amount of a compound disclosed herein or a pharmaceutical composition comprising a compound disclosed herein and a pharmaceutically acceptable carrier. In one embodiment, the cancer is lung cancer, breast cancer, glioma, squamous cell carcinoma, or prostate cancer. In another embodiment, the cancer is non-small cell lung cancer (NSCLC).

In yet another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

The disclosure also provides a kit comprising a compound capable of inhibiting EGFR activity selected from a compound of the present disclosure, or a pharmaceutically acceptable salt thereof, and instructions for use in treating cancer. In one embodiment, the kit further comprises components for performing a test to determine whether a subject has an activating mutation in EGFR or a resistance mutation in EGFR.

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of +20% or +10%, including+5%, +1%, and +0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with wild-type or mutant EGFR an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the disclosure and a co-agent, are both administered to a patient simultaneously in the form of a single entity or dosage. The term “non-fixed combination” means that the active ingredients, e.g. a compound of the disclosure and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “EGFR” refers to epidermal growth factor receptor (alternately referred to as ErbB-1 or HER1) and may refer to the wild-type receptor or to a receptor containing one or more mutations.

As used herein, the term “HER” or Her” refers to members of the ErbB receptor tyrosine kinase family, including EGFR, ERBB2, HER3, and HER4.

As used herein, the term “allosteric site” refers to a site on EGFR other than the ATP binding site, such as that characterized in a crystal structure of EGFR. An “allosteric site” can be a site that is close to the ATP binding site, such as that characterized in a crystal structure of EGFR. For example, one allosteric site includes one or more of the following amino acid residues of epidermal growth factor receptor (EGFR): Lys745, Leu788, Ala743, Cys755, Leu777, Phe856, Asp855, Met766, IIe759, Glu762, and/or Ala763.

As used herein, the term “agent that prevents EGFR dimer formation,” or iterations thereof, refers to an agent that prevents dimer formation in which the C-lobe of the “activator” subunit impinges on the N-lobe of the “receiver” subunit. Examples of agents that prevent EGFR dimer formation include, but are not limited to, cetuximab, trastuzumab, panitumumab, and Mig6.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “haloalkyl” refers to an alkyl group, as defined above, substituted with one or more halo substituents, wherein alkyl and halo are as defined herein. Haloalkyl includes, by way of example, chloromethyl, trifluoromethyl, bromoethyl, chlorofluoroethyl, and the like.

As used herein, the term “alkoxy” refers to the group-O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. In an embodiment, “cycloalkyl” is C3-C10 cycloalkyl. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl.

As used herein, the term “bicyclic ring” means a fused ring system comprising two rings, wherein the first ring is aryl or heteroaryl and the second ring is cycloalkyl or heterocycloalkyl. The term “bicyclic ring” includes, but is not limited to, indoline, isoindoline-1,3-dione, isoindolin-1-one, and dihydro-indene. In an embodiment, the bicyclic ring is indoline.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. In an embodiment, “heterocyclyl” or “heterocycloalkyl” is 3-10 membered heterocycloalkyl. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydro-furanyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo-[2.1.1]hexanyl, 6-azabicyclo[3.1.1]heptanyl, 2-azabicyclo[2.2.1]heptanyl, 3-azabicyclo[3.1.1]-heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]-heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl.

As used herein, the term “heterocycloalkenyl” refers to monounsaturated or polyunsaturated monocyclic carbocycles containing at least one heteroatom selected from oxygen, sulfur, and nitrogen and may comprise benzo-fused analogues thereof. The term “heterocycloalkenyl” specifically includes, but is not limited to dihydropyranyl, dihydrothiopyranyl, dihydrothiophenyl, and tetrahydropyridinyl.

As used herein, the term “aromatic” refers to a carbocycle or heterocycle with one or more polyunsaturated rings and having aromatic character, i.e., having (4n+2) delocalized TT (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. In an embodiment, “aryl” is C6-C10 aryl. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. In an embodiment, “heteroaryl” is 5-10 membered heteroaryl. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl, heteroaryl, cycloalkyl, bicyclic ring, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

Provided herein are compounds that are inhibitors of epidermal growth factor receptor (EGFR) useful in the treatment of kinase-mediated disorders, including cancer and other proliferation diseases. In an embodiment, the compounds provided herein are mutant selective EGFR inhibitors.

In an aspect, provided herein is a compound of Formula A-I:

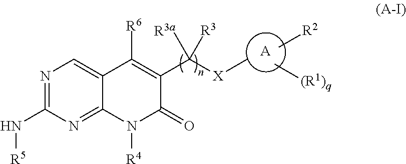

or a pharmaceutically acceptable salt thereof;
   wherein:
X is O, S, NH, CH₂, or a bond;
A is selected from the group consisting of C6-C10 aryl, 5-10 membered heteroaryl, C3-C10 cycloalkyl, 3-10 membered heterocycloalkyl, C₃-C10 cycloalkenyl, 3-10 membered heterocycloalkenyl, and 6-11 membered bicyclic ring;
each R¹ is independently selected from the group consisting of H, ═O, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, NS(O)(C₁-C₆ alkyl)₂, NS(NH)(C₁-C₆ alkyl)₂, NS(NC₁-C₆ alkyl) (C₁-C₆ alkyl)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
q is 1, 2, or 3;
R³ and R^{3a} are each independently selected from the group consisting of H, halo, CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with R⁹;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted with one or two R⁸;
R⁵ is selected from the group consisting of H, C₁-C₆ alkyl, C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl, 6-11 membered bicyclic ring, wherein 3-10 membered heterocycloalkyl, 6-11 membered bicyclic ring, C₆-C₁₀ aryl, and 5-10 membered heteroaryl are each optionally substituted with one, two, or three R^{5a};
each R^{5a} is independently selected from the group consisting of ═O, 3-10 membered heterocycloalkyl, O-3-10 membered heterocycloalkyl, halo, NS(O)(C₁-C₆ alkyl)₂, NS(NH)(C₁-C₆ alkyl)₂, NS(NC₁-C₆ alkyl) (C₁-C₆ alkyl)₂, SO₂C₁-C₆ alkyl, C₁-C₆ alkyl, C₁-C₆ alkoxy, and C₃-C₁₀ cycloalkyl, wherein 3-10 membered heterocycloalkyl, O-3-10 membered heterocycloalkyl, C₁-C₆ alkyl, and C₁-C₆ alkoxy are optionally substituted with one, two, or three R^{5aa};
each R^{5aa} is independently selected from the group consisting of ═O, C₁-C₆ alkyl, C₁-C₆ haloalkyl, halo, CN, OH, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-10 membered heterocycloalkyl, wherein 3-10 membered heterocycloalkyl is optionally substituted with one or two C₁-C₆ alkyl or SO₂C₁-C₆ alkyl;
R⁶ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
each R⁸ is independently selected from halo, C₁-C₆ alkyl, C₁-C₆ alkoxy, C₁-C₆ haloalkoxy, C₃-C₁₀ cycloalkyl, 3-10 membered heterocycloalkyl, O-3-10 membered heterocycloalkyl, C₆-C₁₀ aryl, 5-10 membered heteroaryl, OH, CN, NH₂, NH(C₁-C₆ alkyl), N(C₁-C₆ alkyl)₂, C₁-C₆ alkyl-OH, C(O)C₁-C₆ alkyl-OH, SO₂C₁-C₆ alkyl, SO₂C₃-C₆ cycloalkyl, C(O)C₁-C₆ alkyl, C(O)NHC₁-C₆ alkyl, NHC(O)C₁-C₆ alkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl), wherein C₁-C₆ alkyl, C₁-C₆ alkoxy, 3-10 membered heterocycloalkyl, O-3-10 membered heterocycloalkyl, NH(C₁-C₆ alkyl), and N(C₁-C₆ alkyl)₂ are optionally substituted with one or two R^{8a}; R^{8a} is selected from the group consisting of C₁-C₆ alkyl, C₁-C₆ alkoxy, OH, NH₂, NH(C₁-C₆ alkyl), N(C₁-C₆ alkyl)₂, and 3-10 membered heterocycloalkyl, wherein 3-10 membered heterocycloalkyl is optionally substituted with one or two C₁-C₆ alkyl and C₁-C₆ alkoxy; each R⁹ is independently selected from the group consisting of halo, CN, OH, C₁-C₆ alkyl, OC₁-C₆ alkoxy, NH₂, NH(C₁-C₆ alkyl), and N(C₁-C₆ alkyl)₂;
R² is selected from the group consisting of:

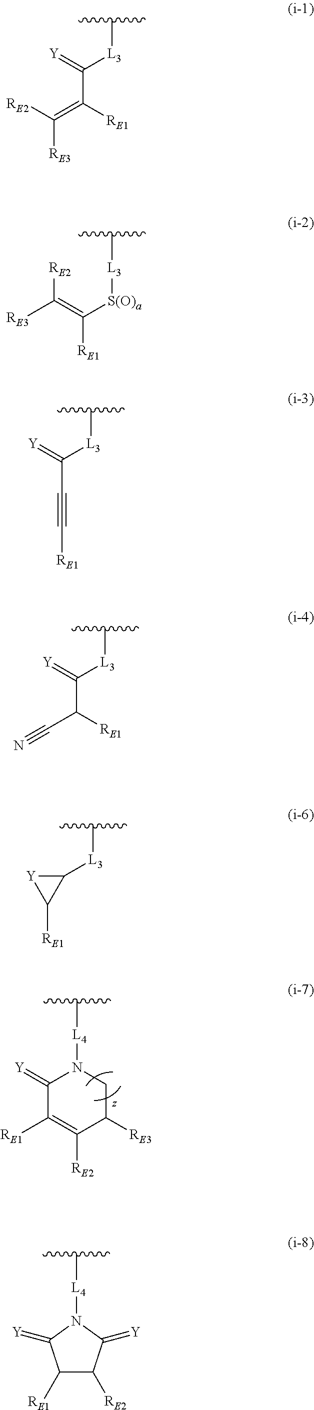

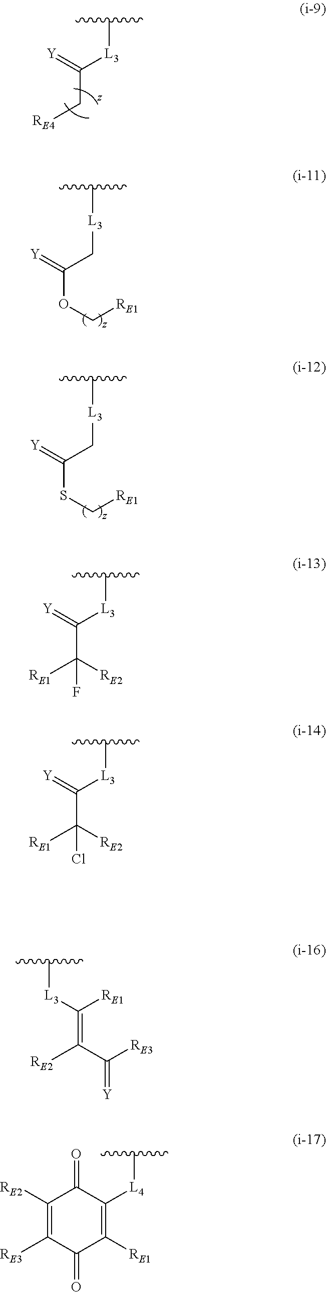

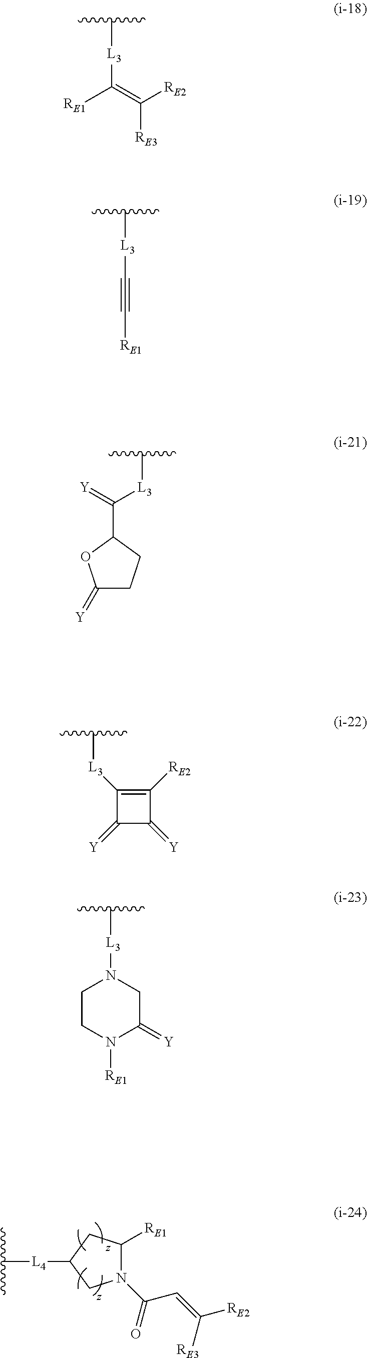

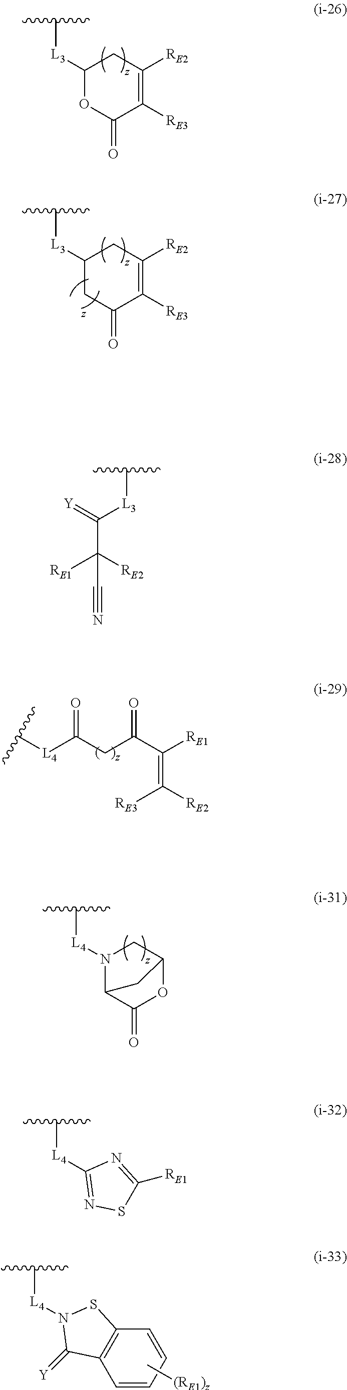

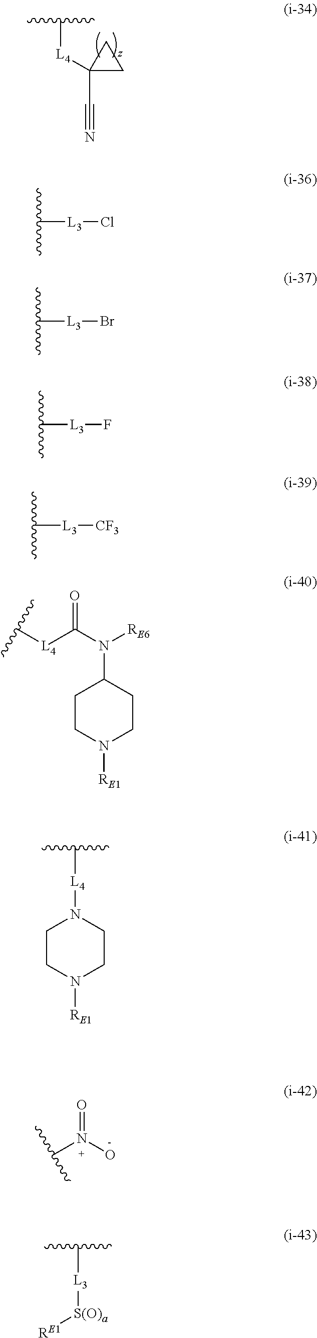

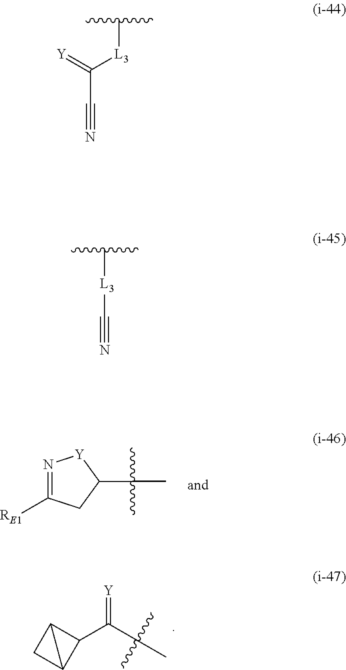

L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S) NR_{L3a}—, trans —CR_{L3b}═CR_{L3b}—, cis —CR_{L3b}═CR_{L3b}—, —C═C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R^{EE})₂, CH₂SR_{EE}, OR_{EE}, N(R^{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

In an embodiment of Formula A-I,
X is O, S, NH, CH₂ or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
each R¹ is independently selected from the group consisting of H, ═O, halo, CN, OR⁷, C₁-C₆ alkyl, C₁-C₆ haloalkyl, N(R⁷)₂, C₆-C₁₀ aryl, and 5-10 membered heteroaryl;
R³ and R^{3a} are each independently selected from the group consisting of H, halo, and C₁-C₆ alkyl;
q is 1, 2, or 3;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, heterocycloalkyl, and heteroaryl are each optionally substituted with one R⁸, and wherein cycloalkyl and aryl are optionally substituted with one or two R⁸;
R⁵ is selected from the group consisting of H, C₁-C₆ alkyl, C₆-C₁₀ aryl, 5-10 membered heteroaryl, C₃-C₁₀ cycloalkyl, 3-10 membered heterocycloalkyl, and 6-11 membered bicyclic ring, wherein C₆-C₁₀ aryl, 5-10 membered heteroaryl, and 6-11 membered bicyclic ring are each optionally substituted with one, two, or three R^{5a};
each R^{5a} is independently selected from ═O, 3-10 membered heterocycloalkyl, O-3-10 membered heterocycloalkyl, halo, SO₂C₁-C₆ alkyl, C₁-C₆ alkyl, and C₁-C₆ alkoxy, wherein O-3-10 membered heterocycloalkyl, C₁-C₆ alkyl, and C₁-C₆ alkoxy are optionally substituted with one R^{5aa} and 3-10 membered heterocycloalkyl is optionally substituted with one or two R^{5aa};
each R^{5aa} is independently selected from ═O, C₁-C₆ alkyl, C₁-C₆ haloalkyl, halo, CN, OH, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-10 membered heterocycloalkyl, wherein 3-10 membered heterocycloalkyl is optionally substituted with one C₁-C₆ alkyl or SO₂C₁-C₆ alkyl;
R⁶ is selected from the group consisting of H, halo, OH, CN, C₁-C₆ alkyl, and C₁-C₆ haloalkyl;
each R⁷ is independently H or C₁-C₆ alkyl;
each R⁸ is independently selected from OH, halo, C₁-C₆ alkyl, C₁-C₆ alkoxy, C₁-C₆ haloalkoxy, 3-10 membered heterocycloalkyl, O-3-10 membered heterocycloalkyl, NH₂, NH(C₁-C₆ alkyl), N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-10 membered heterocycloalkyl, O-3-10 membered heterocycloalkyl, NH(C₁-C₆ alkyl), and N(C₁-C₆ alkyl)₂ are optionally substituted with one or two R^{8a};
R^{8a} is selected from the group consisting of C₁-C₆ alkyl, C₁-C₆ alkoxy, OH, NH₂, NH(C₁-C₆ alkyl), N(C₁-C₆ alkyl)₂, and 3-10 membered heterocycloalkyl, wherein 3-10 membered heterocycloalkyl is optionally substituted with one C₁-C₆ alkyl or C₁-C₆ alkoxy;
R² is selected from the group consisting of:

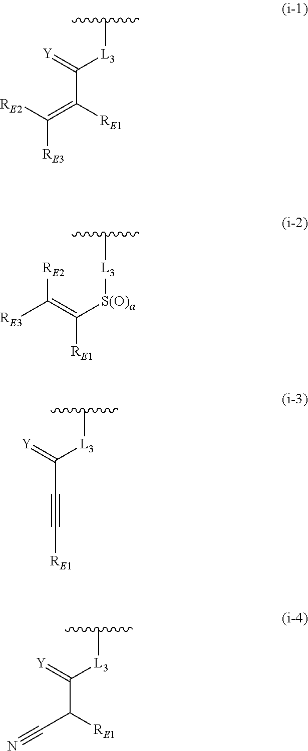

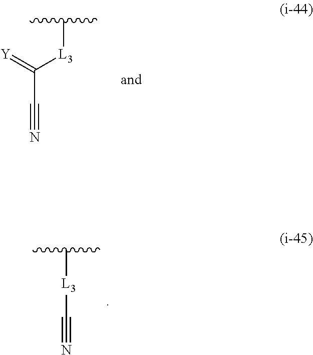

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl;
each Y is independently O, S, or CH₂; and
a is 0, 1, or 2.

In another embodiment of Formula A-I,
X is O, S, or a bond;
A is selected from the group consisting of C₆-C₁₀ aryl, 5-10 membered heteroaryl, 3-10 membered heterocycloalkyl, and 6-10 membered bicyclic ring;
each R¹ is independently selected from the group consisting of H, ═O, halo, CN, OH, C₁-C₆ alkyl, C₁-C₆ alkoxy, C₁-C₆ haloalkyl, NH₂, and phenyl;
q is 1, 2, or 3;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₆ cycloalkyl, 3-6 membered heterocycloalkyl, C₆-C₈ aryl, and 6-8 membered heteroaryl, wherein alkyl, heterocycloalkyl, and heteroaryl are each optionally substituted with one R⁸, and wherein cycloalkyl and aryl are optionally substituted with one or two R⁸;
R⁵ is selected from the group consisting of H, C₁-C₆ alkyl, C₆-C₈ aryl, 5-7 membered heteroaryl, C₃-C₆ cycloalkyl, 3-6 membered heterocycloalkyl, and 8-11 membered bicyclic ring, wherein C₆-C₁₀ aryl, 5-7 membered heteroaryl, and 8-11 membered bicyclic ring are each optionally substituted with one or two R^{5a};
each R^{5a} is independently selected from ═O, 3-6 membered heterocycloalkyl, O-3-6 membered heterocycloalkyl, halo, SO₂C₁-C₆ alkyl, C₁-C₆ alkyl, and C₁-C₆ alkoxy, wherein 3-6 membered heterocycloalkyl, O-3-6 membered heterocycloalkyl, C₁-C₆ alkyl, and C₁-C₆ alkoxy are optionally substituted with one R^{5aa} and 3-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, halo, and CN;
each R⁸ is independently selected from OH, halo, C₁-C₆ alkyl, C₁-C₆ alkoxy, C₁-C₆ haloalkoxy, 3-6 membered heterocycloalkyl, O-3-6 membered heterocycloalkyl, NH₂, NH(C₁-C₆ alkyl), N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-6 membered heterocycloalkyl, O-3-6 membered heterocycloalkyl, NH(C₁-C₆ alkyl), and N(C₁-C₆ alkyl)₂ are optionally substituted with one R^{8a};
R^{8a} is selected from the group consisting of C₁-C₆ alkyl, C₁-C₆ alkoxy, OH, NH₂, NH(C₁-C₆ alkyl), N(C₁-C₆ alkyl)₂, and 3-6 membered heterocycloalkyl, wherein 3-6 membered heterocycloalkyl is optionally substituted with one C₁-C₆ alkyl or C₁-C₆ alkoxy;
R² is selected from the group consisting of:

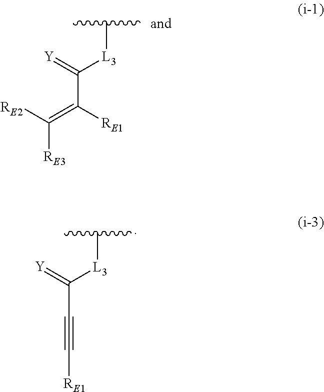

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In yet another embodiment of Formula A-I,
X is O, S, or a bond;
A is selected from the group consisting of phenyl, indole, piperazine, indoline, piperidine, tetrahydroquinoxaline, quinoxaline, tetrahydroquinoline, quinoline, azaspiro[2.5]octane, azaspiro[3.5]nonane, azaspiro[4.5]decane, diazaspiro[2.5]octane, diazaspiro[3.5]nonane, diazaspiro[4.5]decane, 2,5-diazabicyclo[4.1.0]heptane, diazabicyclo[4.2.0]octane, octahydro-1H-cyclopenta[c]pyridine, octahydro-1H-cyclopenta[b]pyrazine, and octahydro-1H-pyrrolo[3,4-c]pyridine;
each R¹ is independently selected from the group consisting of H, C₁-C₃ alkyl, and C₁-C₃ haloalkyl;
q is 1, 2, or 3;
R³ and R^{3a} are each independently selected from the group consisting of H, halo, and

C₁-C₃ alkyl;
n is 0 or 1;
R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸;
R⁵ is selected from the group consisting of H, C₁-C₆ alkyl, C₆-C₁₀ aryl, 5-10 membered heteroaryl, C₃-C₆ cycloalkyl, 9-10 membered bicyclic ring, and 4-5 membered heterocycloalkyl, wherein C₆-C₁₀ aryl, 5-10 membered heteroaryl, and 9-10 membered bicyclic ring are each optionally substituted with one or two R^{5a};
each R^{5a} is independently selected from ═O, 3-6 membered heterocycloalkyl, O-3-6 membered heterocycloalkyl, halo, SO₂C₁-C₆ alkyl, C₁-C₆ alkyl, and C₁-C₆ alkoxy, wherein 3-6 membered heterocycloalkyl, O-3-6 membered heterocycloalkyl, C₁-C₆ alkyl, and C₁-C₆ alkoxy are optionally substituted with one R^{5aa} and 3-6 membered heterocycloalkyl is optionally substituted with one or two R^{5aa}:
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, C₁-C₃ alkyl, and C₁-C₃ haloalkyl;
each R⁸ is independently selected from OH, halo, C₁-C₆ alkyl, C₁-C₆ alkoxy, C₁-C₆ haloalkoxy, 3-6 membered heterocycloalkyl, O-3-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-6 membered heterocycloalkyl, and N(C₁-C₆ alkyl)₂ are optionally substituted with one R^{8a};
R^{8a} is selected from the group consisting of C₁-C₆ alkyl, C₁-C₆ alkoxy, OH, NH₂, NH(C₁-C₆ alkyl), N(C₁-C₆ alkyl)₂, and 3-6 membered heterocycloalkyl, wherein 3-6 membered heterocycloalkyl is optionally substituted with one C₁-C₆ alkyl or C₁-C₆ alkoxy;
R² is selected from the group consisting of:

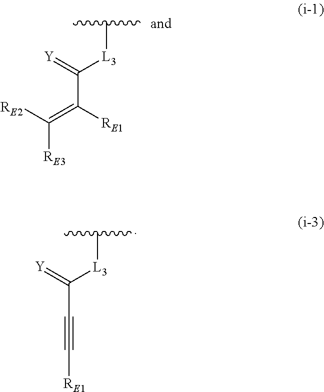

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In still another embodiment of Formula A-I,
X is O, S, or a bond;
A is selected from the group consisting of:

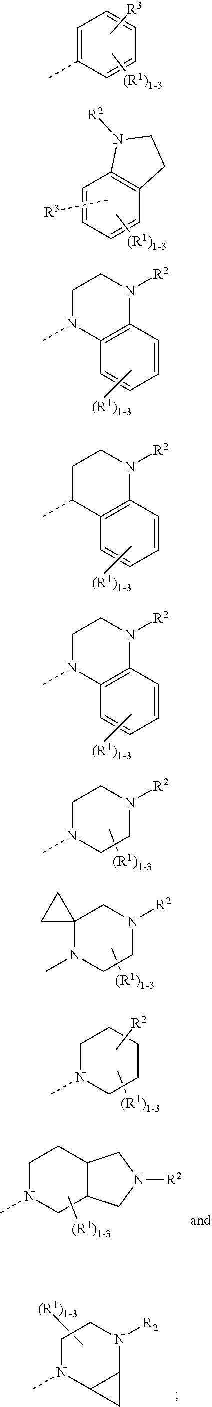

each R¹ is independently selected from the group consisting of H, ═O, halo, OH, C₁-C₆ alkyl, C₁-C₆ alkoxy, NH₂, and phenyl;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸;
R⁵ is selected from the group consisting of H, C₁-C₆ alkyl, phenyl, 5-6 membered heteroaryl, C₃-C₆ cycloalkyl, 9-10 membered bicyclic ring, and 4-5 membered heterocycloalkyl, wherein phenyl, 5-6 membered heteroaryl, and 9-10 membered bicyclic ring are each optionally substituted with one or two R^{5a};
each R^{5a} is independently selected from ═O, 5-6 membered heterocycloalkyl, O-3-4 membered heterocycloalkyl, halo, SO₂C₁-C₆ alkyl, C₁-C₆ alkyl, and C₁-C₆ alkoxy, wherein 5-6 membered heterocycloalkyl, O-3-4 membered heterocycloalkyl, C₁-C₆ alkyl, and C₁-C₆ alkoxy are optionally substituted with one R^{5aa} and 5-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, C₁-C₃ alkyl, and C₁-C₃ haloalkyl;
each R⁸ is independently selected from OH, halo, C₁-C₆ alkyl, C₁-C₆ alkoxy, C₁-C₆ haloalkoxy, 4-6 membered heterocycloalkyl, O-4-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 4-6 membered heterocycloalkyl, and N(C₁-C₆ alkyl)₂ are optionally substituted with one R^{8a};
R^{8a} is selected from the group consisting of C₁-C₆ alkyl, C₁-C₆ alkoxy, OH, NH₂, NH(C₁-C₆ alkyl), N(C₁-C₆ alkyl)₂, and 5-6 membered heterocycloalkyl, wherein 5-6 membered heterocycloalkyl is optionally substituted with one C₁-C₆ alkyl or C₁-C₆ alkoxy;
R² is selected from the group consisting of:

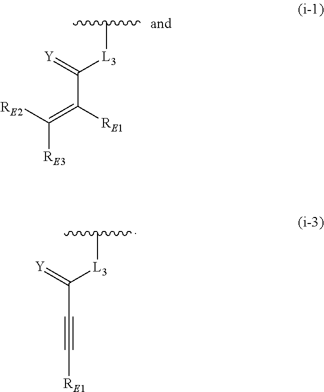

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In an embodiment, the compound of Formula A-I is a compound of Formula I:

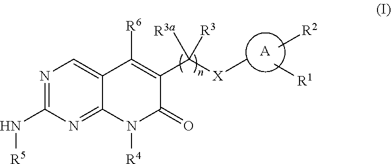

or a pharmaceutically acceptable salt thereof;
   wherein:
X is O, S, NH, CH₂, or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-11 membered bicyclic ring;
R¹ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
R³ and R^{3a} are each independently selected from the group consisting of H, halo, CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with R⁹;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl, 6-11 membered bicyclic ring, wherein 3-10 membered heterocycloalkyl, 6-11 membered bicyclic ring, C₆-C₁₀ aryl, and 5-10 membered heteroaryl are each optionally substituted with one, two, or three R^{5a};
each R^{5a} is independently selected from 3-10 membered heterocycloalkyl, C₁-C₆ alkoxy, and C₃-C₁₀ cycloalkyl, wherein 3-10 membered heterocycloalkyl is optionally substituted with halo, CN, C₁-C₆ alkyl, and C₁-C₆ haloalkyl;
R⁶ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
R⁸ is selected from C₁-C₆ alkyl, C₃-C₁₀ cycloalkyl, 3-10 membered heterocycloalkyl, C₆-C₁₀ aryl, and 5-10 membered heteroaryl, OH, CN, NH₂, NH(C₁-C₆ alkyl), N(C₁-C₆ alkyl)₂, C₁-C₆ alkyl-OH, C(O)C₁-C₆ alkyl-OH, SO₂C₁-C₆ alkyl, SO₂C₃-C₆ cycloalkyl, halo, C(O)C₁-C₆ alkyl, C₁-C₆ alkyl, C₁-C₆ alkoxy, NHC(O)C₁-C₆ alkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl);
each R⁹ is independently selected from the group consisting of halo, CN, OH, C₁-C₆ alkyl, OC₁-C₆ alkoxy, NH₂, NH(C₁-C₆ alkyl), and N(C₁-C₆ alkyl)₂;
R² is selected from the group consisting of:

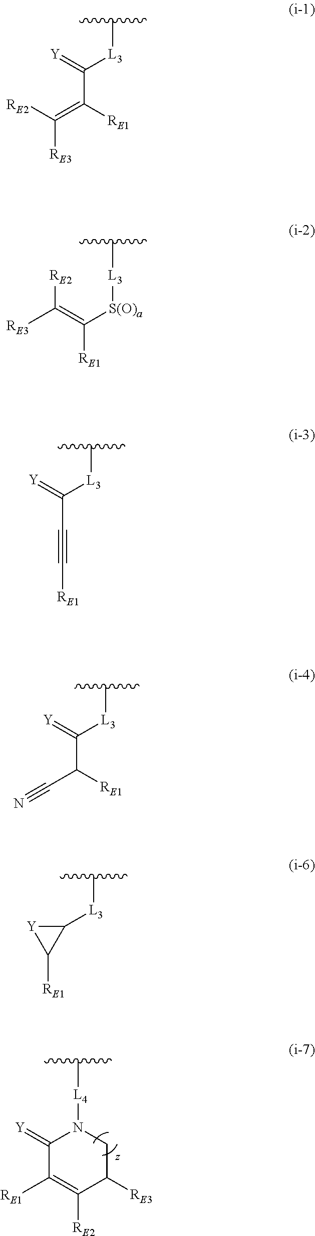

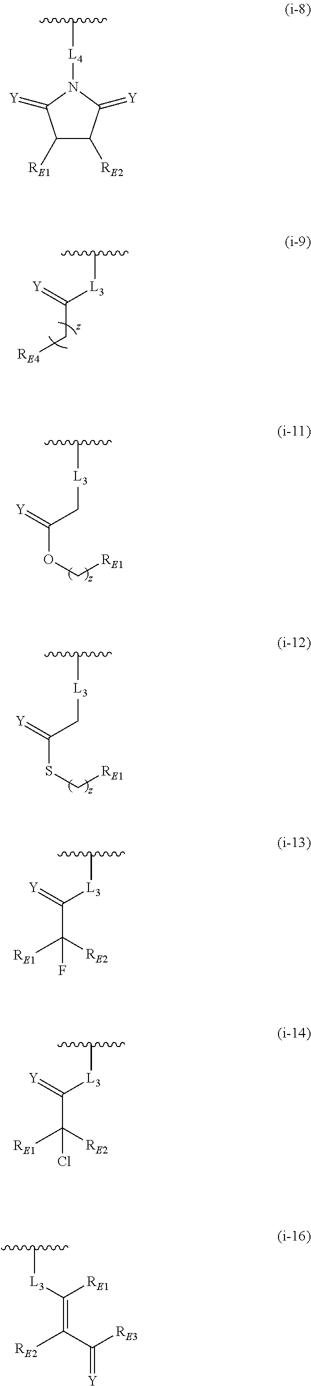

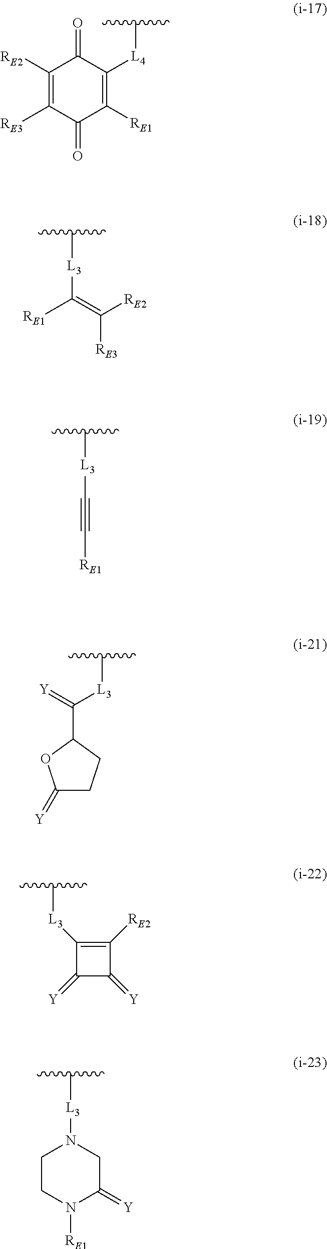

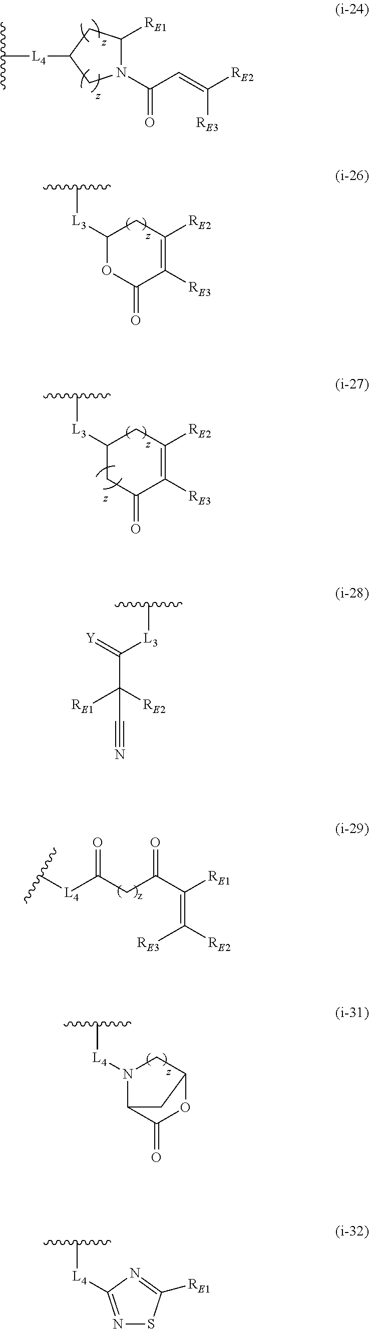

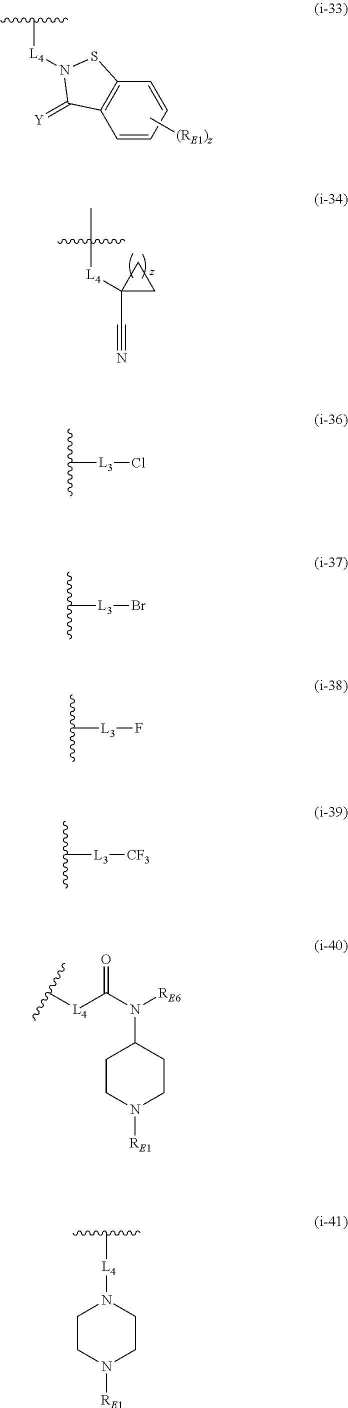

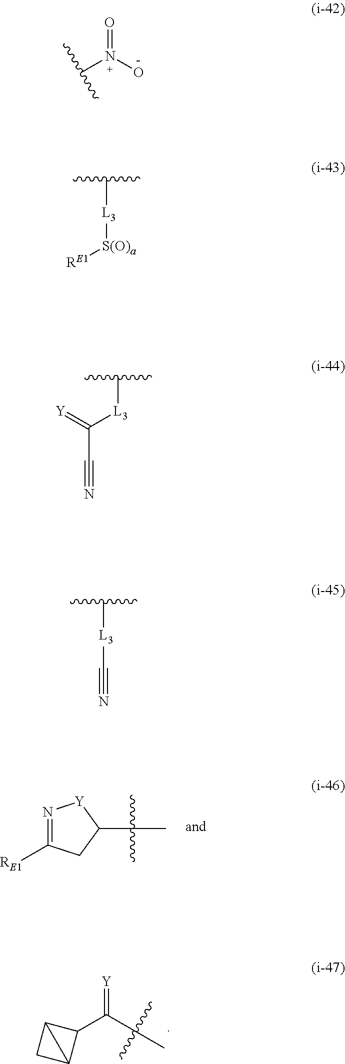

L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S) NR_{L3a}—, trans —CR_{L3b}═CR_{L3b}—, cis —CR_{L3b}═CR_{L3b}—, —C═C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R^{EE})₂, CH₂SR_{EE}, OR_{EE}, N(R^{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

In an embodiment of Formula I,
X is O, S, NH, CH₂ or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
R¹ is selected from the group consisting of H, halo, CN, C₁-C₆ alkyl, and C₁-C₆ haloalkyl;
R³ and R^{3a} are each independently selected from the group consisting of H, halo, and C₁-C₆ alkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, and 3-10 membered heterocycloalkyl, wherein C₆-C₁₀ aryl and 5-10 membered heteroaryl are each optionally substituted with one, two, or three R^{5a};
each R^{5a} is independently selected from 3-10 membered heterocycloalkyl and C₁-C₆ alkoxy, wherein 3-10 membered heterocycloalkyl is optionally substituted with halo, CN, C₁-C₆ alkyl, and C₁-C₆ haloalkyl;
R⁶ is selected from the group consisting of H, halo, CN, C₁-C₆ alkyl, and C₁-C₆ haloalkyl;
R⁸ is selected from C(O)C₁-C₆ alkyl-OH, C(O)C₁-C₆ alkyl, and NHC(O)C₁-C₆ alkyl;
R² is selected from the group consisting of:

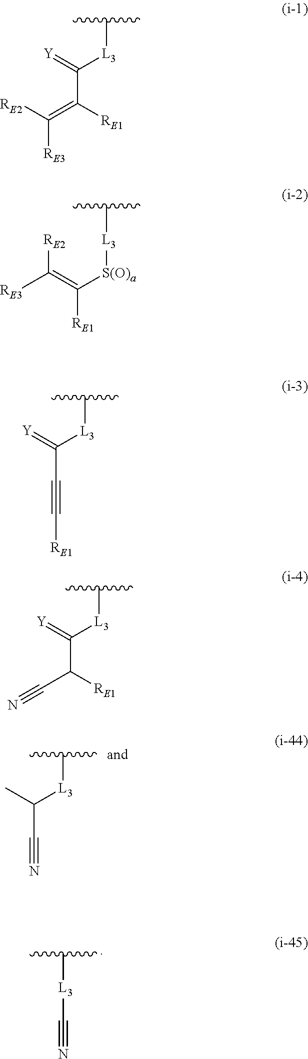

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl;
each Y is independently O, S, or CH₂; and
a is 0, 1, or 2.

In another embodiment of Formula I,
X is O, S, or a bond;
A is selected from the group consisting of C₆-C₁₀ aryl, 5-10 membered heteroaryl, 3-10 membered heterocycloalkyl, and 6-10 membered bicyclic ring;
R¹ is selected from the group consisting of H, halo, CN, C₁-C₆ alkyl, and C₁-C₆ haloalkyl;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, and 3-10 membered heterocycloalkyl, wherein C₆-C₁₀ aryl and 5-10 membered heteroaryl are each optionally substituted with one, two, or three R^{5a};
each R^{5a} is independently selected from 3-10 membered heterocycloalkyl and C₁-C₆ alkoxy, wherein 3-10 membered heterocycloalkyl is optionally substituted with C₁-C₆ alkyl;
R⁶ is selected from the group consisting of H, halo, CN, C₁-C₆ alkyl, and C₁-C₆ haloalkyl;
R⁸ is selected from C(O)C₁-C₆ alkyl-OH, C(O)C₁-C₆ alkyl, and NHC(O)C₁-C₆ alkyl;
R² is selected from the group consisting of:

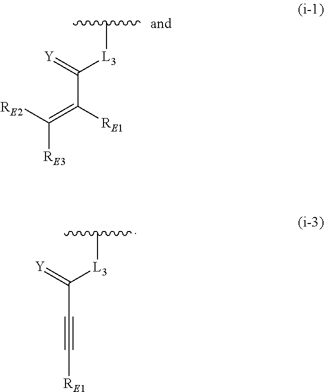

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In yet another embodiment of Formula I,
X is O, S, or a bond;
A is selected from the group consisting of phenyl, indole, piperazine, indoline, piperidine, and tetrahydroquinoxaline;
R¹ is selected from the group consisting of H, C₁-C₃ alkyl, and C₁-C₃ haloalkyl;
R³ and R^{3a} are each independently selected from the group consisting of H, halo, and C₁-C₃ alkyl;
n is 0 or 1;
R⁴ is selected from the group consisting of H, C₁-C₃ alkyl, 6-10 membered heterocycloalkyl, and C₆-C₁₀ aryl; wherein heterocycloalkyl and aryl are each optionally substituted with R⁸;
R⁵ is selected from the group consisting of C₆-C₁₀ aryl and 5-10 membered heteroaryl, wherein C₆-C₁₀ aryl and 5-10 membered heteroaryl are each optionally substituted with one or two R^{5a};
each R^{5a} is independently selected from 6-10 membered heterocycloalkyl and C₁-C₃ alkoxy, wherein the 6-10 membered heterocycloalkyl is optionally substituted with halo, CN, C₁-C₃ alkyl, and C₁-C₃ haloalkyl;
R⁶ is selected from the group consisting of H, C₁-C₃ alkyl, and C₁-C₃ haloalkyl;
R⁸ is selected from C(O)C₁-C₃ alkyl-OH, C(O)C₁-C₃ alkyl, and NHC(O)C₁-C₃ alkyl;
R² is selected from the group consisting of:

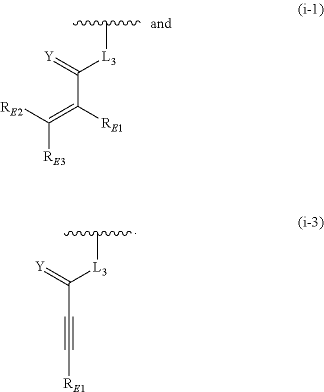

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In still another embodiment, the compound of Formula I is a compound of Formula IA:

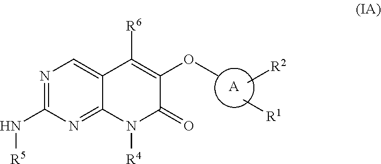

or a pharmaceutically acceptable salt thereof.

In an embodiment, the compound of Formula I is a compound of Formula IB:

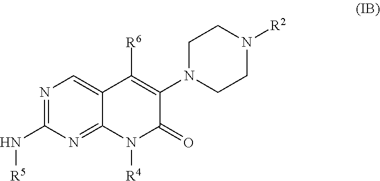

or a pharmaceutically acceptable salt thereof.

In another embodiment, R³ and R^{3a} are each independently H or C₁-C₃ alkyl. In yet another embodiment, X is O or a bond. In still another embodiment, wherein X is O.

In an embodiment, X is a bond. In another embodiment, R³ and R^{3a} are each independently CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with R⁹. In yet another embodiment, R³ and R^{3a} are each independently CN, C₁-C₃ alkyl, C₁-C₃ alkoxy, NH(C₁-C₃ alkyl), and N(C₁-C₃ alkyl)₂, wherein C₁-C₃ alkyl is optionally substituted one or two times with R⁹. In still another embodiment, R³ and R^{3a} are each independently CN, CH₂CN, OCH₃, NH(CH₃), and N(CH₃)₂.

In an embodiment, R⁵ is 3-10 membered heterocycloalkyl or 6-11 membered bicyclic ring, wherein 3-10 membered heterocycloalkyl and 6-11 membered bicyclic ring are optionally substituted with R^{5a}; In another embodiment, R^{5a} is C₃-C₆ cycloalkyl and R⁵ is substituted by cycloalkyl in spiro configuration. In yet another embodiment, A is piperazine or tetrahydroquinoxaline. In still another embodiment R¹ is H or C₁-C₃ alkyl.

In an embodiment, R⁴ is H, C₁-C₃ alkyl, piperidine, and phenyl, wherein piperidine and phenyl are optionally substituted with R⁸. In another embodiment, R⁵ is phenyl and each R^{5a} is independently 6-10 membered heterocycloalkyl or C₁-C₃ alkoxy, wherein 6-10 membered heterocycloalkyl is optionally substituted with C₁-C₃ alkyl.

In yet another embodiment, the compound of Formula I is a compound of Formula IC:

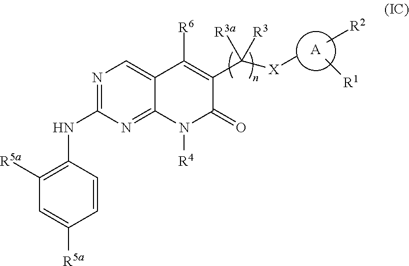

or a pharmaceutically acceptable salt thereof.

In still another embodiment, the compound of Formula I is a compound of Formula ID:

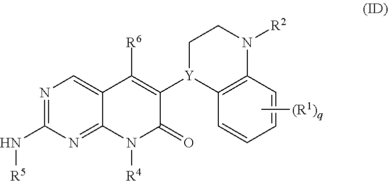

or a pharmaceutically acceptable salt thereof,
   wherein
Y is CH or N.

In an embodiment, the compound of Formula ID is a compound of Formula IDa:

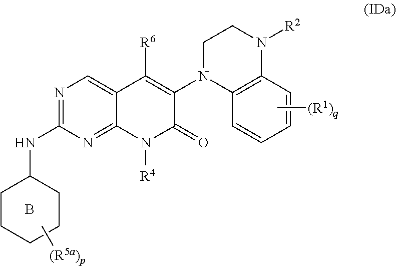

or a pharmaceutically acceptable salt thereof,
   wherein
ring B is selected from the group consisting of cyclohexyl, phenyl, pyridine, pyridazine, pyrimidine, and pyrazine;
and p is 1 or 2.

In another embodiment, the compound of Formula ID is a compound of Formula IDb:

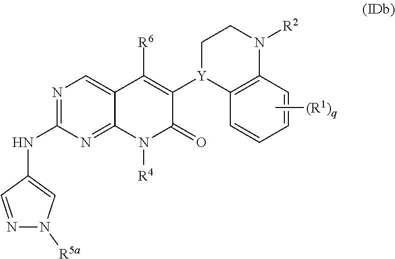

or a pharmaceutically acceptable salt thereof,
   wherein
Y is CH or N.

In yet an embodiment, the compound of Formula I is a compound of Formula IE:

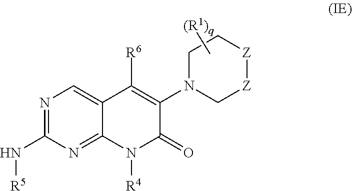

or a pharmaceutically acceptable salt thereof,
   wherein
each Z is independently selected from the group consisting of NR², CHR², NH, and CH2, wherein only one Z is NR² or NH, and at least one Z is NR² or CHR².

In still another embodiment, the compound of Formula IE is a compound of Formula IEa:

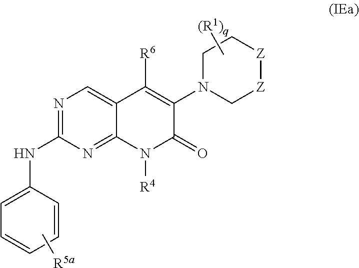

or a pharmaceutically acceptable salt thereof,
   wherein
NR², CHR², NH, and CH₂, wherein only one Z is NR² or NH, and at least one Z is NR² or CHR².

In an embodiment, the compound of Formula IE is a compound of Formula IEb:

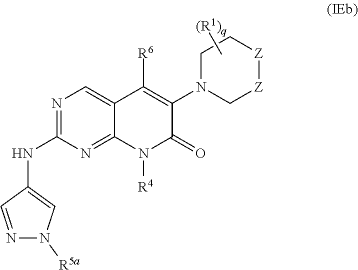

or a pharmaceutically acceptable salt thereof, wherein
NR², CHR², NH, and CH₂, wherein only one Z is NR² or NH, and at least one Z is NR² or CHR².

In another embodiment, the compound of Formula I is a compound of Formula IF:

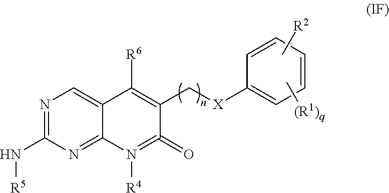

or a pharmaceutically acceptable salt thereof.

In yet another embodiment, the compound of Formula IF is a compound of Formula IFa:

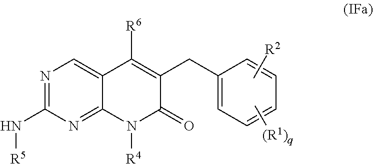

or a pharmaceutically acceptable salt thereof.

In another embodiment, the compound of Formula IFa is a compound of Formula IFaa:

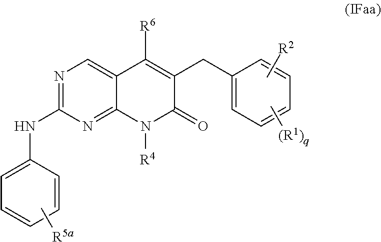

or a pharmaceutically acceptable salt thereof, wherein the variables are defined herein.

In still another embodiment, the compound of Formula IF is a compound of Formula IFb:

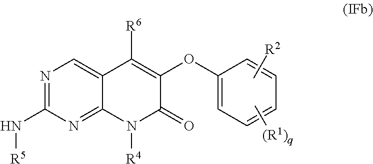

or a pharmaceutically acceptable salt thereof.

In yet another embodiment, the compound of Formula IFb is a compound of Formula

IFba:

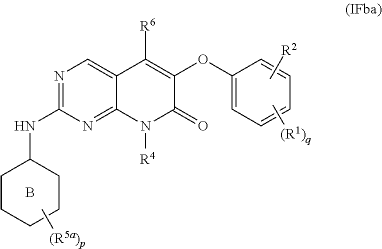

or a pharmaceutically acceptable salt thereof,
   wherein
ring B is selected from the group consisting of cyclohexyl, phenyl, pyridine, pyridazine, pyrimidine, and pyrazine;
and p is 1 or 2.

### Various Embodiments of Formula A-I through Formula IFba

In an embodiment,
X is O, S, or a bond;
R¹ is selected from the group consisting of H, ═O, halo, OH, C₁-C₆ alkyl, C₁-C₆ alkoxy, NH₂, and phenyl;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸;
R⁵ is selected from the group consisting of H, C₁-C₆ alkyl, phenyl, 5-6 membered heteroaryl, C₃-C₆ cycloalkyl, 4-membered heterocycloalkyl, and 9-10 membered bicyclic ring, wherein phenyl, 5-6 membered heteroaryl, and 9-10 membered bicyclic ring are each optionally substituted with one or two R^{5a};
each R^{5a} is independently selected from ═O, 5-6 membered heterocycloalkyl, O-3-4 membered heterocycloalkyl, halo, SO₂C₁-C₆ alkyl, C₁-C₆ alkyl, and C₁-C₆ alkoxy, wherein 5-6 membered heterocycloalkyl, O-3-4 membered heterocycloalkyl, C₁-C₆ alkyl, and C₁-C₆ alkoxy are optionally substituted with one R^{5aa} and 5-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, C₁-C₃ alkyl, and C₁-C₃ haloalkyl;
each R⁸ is independently selected from OH, halo, C₁-C₆ alkyl, C₁-C₆ alkoxy, C₁-C₆ haloalkoxy, 4-6 membered heterocycloalkyl, O-4-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 4-6 membered heterocycloalkyl, and N(C₁-C₆ alkyl)₂ are optionally substituted with one R^{8a};
R^{8a} is selected from the group consisting of C₁-C₆ alkyl, C₁-C₆ alkoxy, OH, NH₂, NH(C₁-C₆ alkyl), N(C₁-C₆ alkyl)₂, and 5-6 membered heterocycloalkyl, wherein 5-6 membered heterocycloalkyl is optionally substituted with one C₁-C₆ alkyl or C₁-C₆ alkoxy;
R² is selected from the group consisting of:

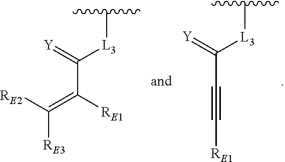

L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

In another embodiment, R⁴ is C₁-C₆ alkyl optionally substituted with one R⁸, or is selected from the group consisting of:

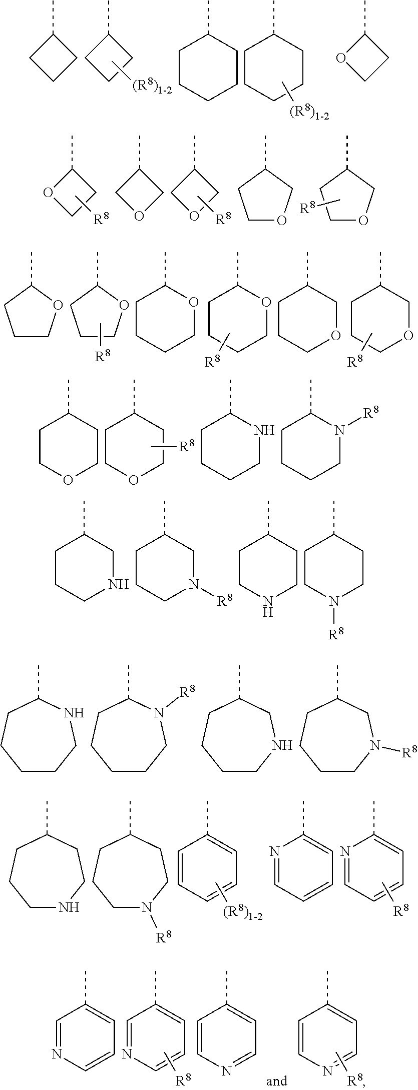

wherein,
each R⁸ is independently selected from OH, halo, C₁-C₆ alkyl, C₁-C₆ alkoxy, C₁-C₆ haloalkoxy, 4-6 membered heterocycloalkyl, O-4-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 4-6 membered heterocycloalkyl, and N(C₁-C₆ alkyl)₂ are optionally substituted with one R^{8aa}; and
R^{8a} is selected from the group consisting of C₁-C₆ alkyl, C₁-C₆ alkoxy, OH, NH₂, NH(C₁-C₆ alkyl), N(C₁-C₆ alkyl)₂, and 5-6 membered heterocycloalkyl, wherein 5-6 membered heterocycloalkyl is optionally substituted with one C₁-C₆ alkyl or C₁-C₆ alkoxy.

In yet another embodiment, R⁵ is H, C₁-C₆ alkyl, or is selected from the group consisting of:

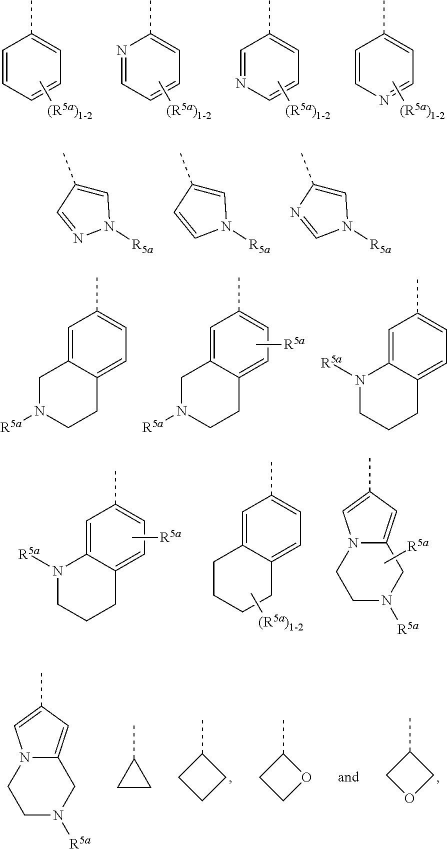

wherein,
each R^{5a} is independently selected from ═O, 5-6 membered heterocycloalkyl, O-3-4 membered heterocycloalkyl, halo, SO₂C₁-C₆ alkyl, C₁-C₆ alkyl, and C₁-C₆ alkoxy, wherein 5-6 membered heterocycloalkyl, O-3-4 membered heterocycloalkyl, C₁-C₆ alkyl, and C₁-C₆ alkoxy are optionally substituted with one R^{5aa} and 5-6 membered heterocycloalkyl is optionally substituted with one or two R^{5aa}; and
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl.

In an embodiment, X is O or a bond.

In an embodiment, A is selected from the group consisting of phenyl, indole, piperazine, indoline, piperidine, tetrahydroquinoxaline, quinoxaline, tetrahydroquinoline, quinoline, azaspiro[2.5]octane, azaspiro[3.5]nonane, azaspiro[4.5]decane, diazaspiro[2.5]octane, diazaspiro[3.5]nonane, diazaspiro[4.5]decane, 2,5-diazabicyclo[4.1.0]heptane, diazabicyclo[4.2.0]octane, octahydro-1H-cyclopenta[c]pyridine, octahydro-1H-cyclopenta[b]pyrazine, and octahydro-1H-pyrrolo[3,4-c]pyridine.

In another embodiment, A is selected from the group consisting of:

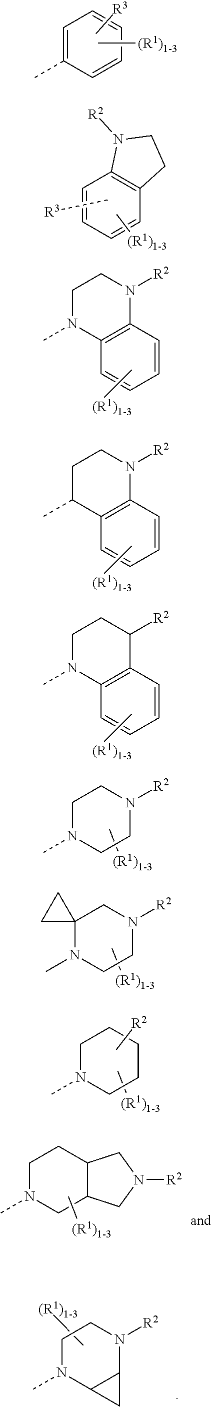

In yet another embodiment, each R¹ is independently selected from the group consisting of H, C₁-C₆ alkyl, C₁-C₆ haloalkyl, NS(O)(C₁-C₆ alkyl)₂, NS(NH)(C₁-C₆ alkyl)₂, and NS(NC₁-C₆ alkyl) (C₁-C₆ alkyl)₂. In another embodiment, each R¹ is independently selected from the group consisting of H, C₁-C₆ alkyl, and C₁-C₆ haloalkyl. In still another embodiment, each R¹ is independently selected from the group consisting of H, C₁-C₃ alkyl, and C₁-C₃ haloalkyl. In an embodiment, q is 1, 2, or 3. In another embodiment, q is 1 or 2.

In yet another embodiment, R³ and R^{3a} are each independently selected from the group consisting of H, halo, and C₁-C₆ alkyl. In still another embodiment, R³ and R^{3a} are each independently selected from the group consisting of H, halo, and C₁-C₃ alkyl. In an embodiment, n is 0. In another embodiment, n is 1.

In yet another embodiment, R⁴ is selected from the group consisting of H, C₁-C₆ alkyl, C₃-C₆ cycloalkyl, 3-6 membered heterocycloalkyl, C₆-C₈ aryl, and 6-8 membered heteroaryl, wherein alkyl, heterocycloalkyl, and heteroaryl are each optionally substituted with one R⁸, and wherein cycloalkyl and aryl are optionally substituted with one or two R⁸.

In still another embodiment, R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸.

In an embodiment, R⁵ is selected from the group consisting of H, C₁-C₆ alkyl, phenyl, 5-6 membered heteroaryl, C₃-C₆ cycloalkyl, 9-10 membered bicyclic ring, and 4-5 membered heterocycloalkyl, wherein phenyl, 5-6 membered heteroaryl, and 9-10 membered bicyclic ring are each optionally substituted with one or two R^{5a}.

In another embodiment, R⁵ is H, C₁-C₆ alkyl, or is selected from the group consisting of:

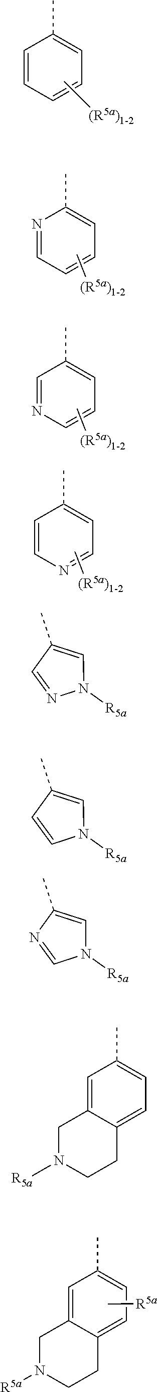

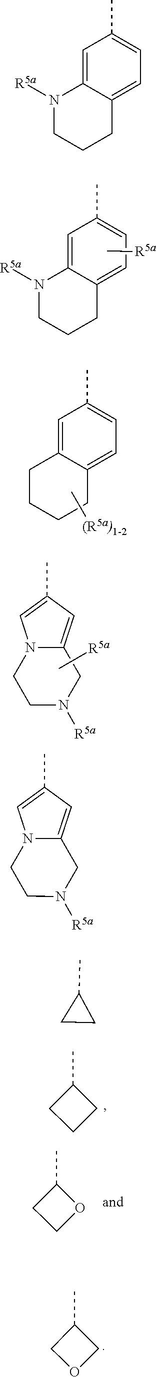

In yet another embodiment, each R^{5a} is independently selected from ═O, 3-6 membered heterocycloalkyl, O-3-6 membered heterocycloalkyl, halo, SO₂C₁-C₆ alkyl, C₁-C₆ alkyl, and C₁-C₆ alkoxy, wherein 3-6 membered heterocycloalkyl, O-3-6 membered heterocycloalkyl, C₁-C₆ alkyl, and C₁-C₆ alkoxy are optionally substituted with one R^{5aa} and 3-6 membered heterocycloalkyl is optionally substituted with one or two R^{5aa}.

In still another embodiment, each R^{5a} is independently selected from ═O, 5-6 membered heterocycloalkyl, O-3-4 membered heterocycloalkyl, halo, SO₂C₁-C₆ alkyl, C₁-C₆ alkyl, and C₁-C₆ alkoxy, wherein 5-6 membered heterocycloalkyl, O-3-4 membered heterocycloalkyl, C₁-C₆ alkyl, and C₁-C₆ alkoxy are optionally substituted with one R^{5aa} and 5-6 membered heterocycloalkyl is optionally substituted with one or two R^{5aa}

In an embodiment, each R^{5a} is individually selected from piperazine and C₁-C₃ alkoxy, wherein piperazine is optionally substituted with C₁-C₃ alkyl.

In another embodiment, each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl.

In yet another embodiment, R⁶ is selected from the group consisting of H, halo, and CN.

In another embodiment, R⁶ is H or C₁-C₃ alkyl. In yet another embodiment, R⁶ is H or halo.

In still another embodiment, each R⁸ is independently selected from OH, halo, C₁-C₆ alkyl, C₁-C₆ alkoxy, C₁-C₆ haloalkoxy, 3-6 membered heterocycloalkyl, O-3-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-6 membered heterocycloalkyl, and N(C₁-C₆ alkyl)₂ are optionally substituted with one R^{8a}; In an embodiment, R⁸ is C(O)C₁-C₆ alkyl, and NHC(O)C₁-C₆ alkyl.

In another embodiment, R^{8a} is selected from the group consisting of C₁-C₆ alkyl, C₁-C₆ alkoxy, OH, NH₂, NH(C₁-C₆ alkyl), N(C₁-C₆ alkyl)₂, C(O)NHC₁-C₆ alkyl, and 3-6 membered heterocycloalkyl, wherein 3-6 membered heterocycloalkyl is optionally substituted with one C₁-C₆ alkyl or C₁-C₆ alkoxy.

In yet another embodiment, R² is selected from the group consisting of:

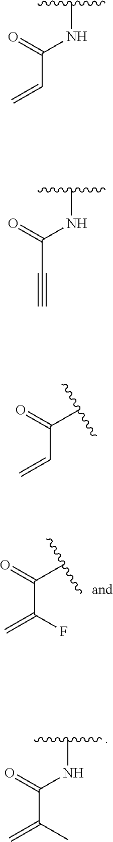

In another embodiment, R² is selected from the group consisting of:

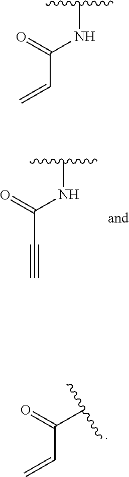

In still another embodiment, R² is selected from the group consisting of:

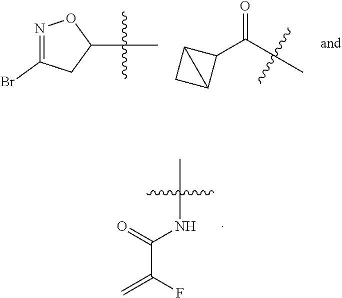

In an embodiment, R⁸ is selected from C₁-C₆ alkyl, C₃-C₁₀ cycloalkyl, 3-10 membered heterocycloalkyl, C₆-C₁₀ aryl, 5-10 membered heteroaryl, OH, CN, NH₂, C₁-C₆ alkyl-OH, 3-10 membered heterocycloalkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl).

In another embodiment, each R⁹ is independently selected from halo, OH, C₁-C₆ alkyl, OC₁-C₆ alkoxy, NH₂, NH(C₁-C₆ alkyl), and N(C₁-C₆ alkyl)₂.

In yet another embodiment, the compound of Formula I is selected from the group consisting of a compound in Table 1

TABLE 1

| Compound | |
|---|---|
| No. | Structure |
| 001 | 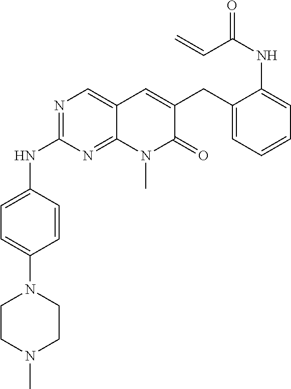 [SMILES: C=CC(=O)Nc1ccccc1Cc1cc2cnc(Nc3ccc(N4CCN(C)CC4)cc3)nc2n(C)c1=O] |
| 002 | 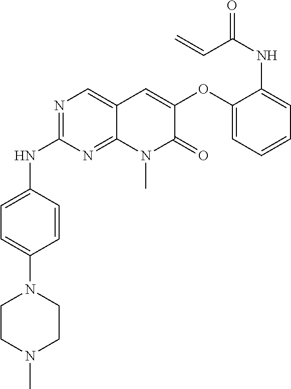 [SMILES: C=CC(=O)Nc1ccccc1Oc1cc2cnc(Nc3ccc(N4CCN(C)CC4)cc3)nc2n(C)c1=O] |
| 003 | 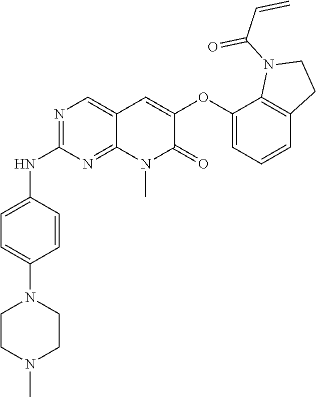 [SMILES: C=CC(=O)N1CCc2cccc(Oc3cc4cnc(Nc5ccc(N6CCN(C)CC6)cc5)nc4n(C)c3=O)c21] |
| 004 | 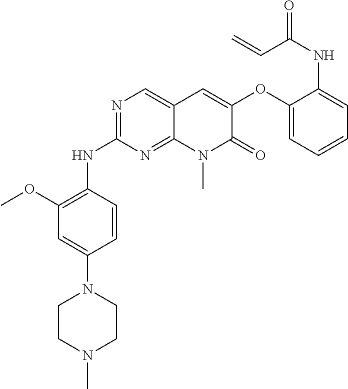 [SMILES: C=CC(=O)Nc1ccccc1Oc1cc2cnc(Nc3ccc(N4CCN(C)CC4)cc3OC)nc2n(C)c1=O] |
| 005 | 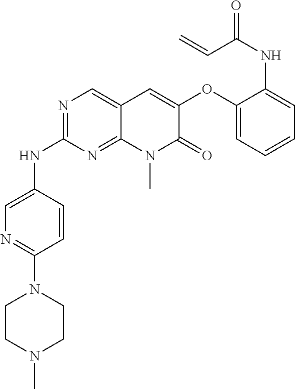 [SMILES: C=CC(=O)Nc1ccccc1Oc1cc2cnc(Nc3ccc(N4CCN(C)CC4)nc3)nc2n(C)c1=O] |
| 006 | 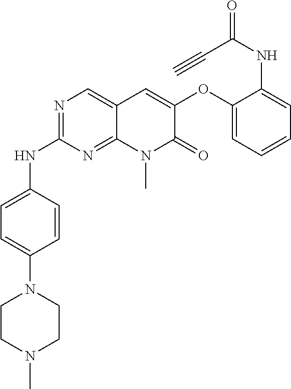 [SMILES: C#CC(=O)Nc1ccccc1Oc1cc2cnc(Nc3ccc(N4CCN(C)CC4)cc3)nc2n(C)c1=O] |
| 007 | 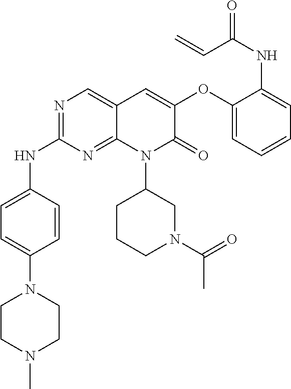 [SMILES: C=CC(=O)Nc1ccccc1Oc1cc2cnc(Nc3ccc(N4CCN(C)CC4)cc3)nc2n(C2CCCN(C(C)=O)C2)c1=O] |
| 008 | 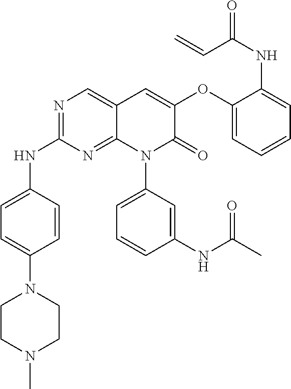 [SMILES: C=CC(=O)Nc1ccccc1Oc1cc2cnc(Nc3ccc(N4CCN(C)CC4)cc3)nc2n(-c2cccc(NC(C)=O)c2)c1=O] |
| 009 | 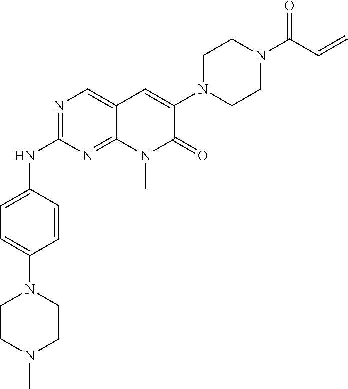 [SMILES: C=CC(=O)N1CCN(c2cc3cnc(Nc4ccc(N5CCN(C)CC5)cc4)nc3n(C)c2=O)CC1] |
| 010 | 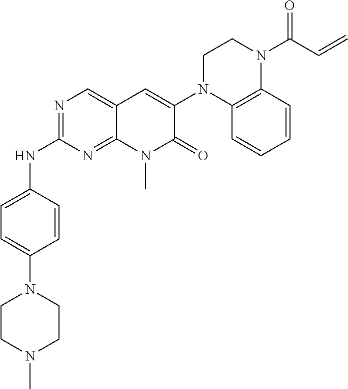 [SMILES: C=CC(=O)N1CCN(c2cc3cnc(Nc4ccc(N5CCN(C)CC5)cc4)nc3n(C)c2=O)c2ccccc21] |

or a pharmaceutically acceptable salt thereof.

In still another embodiment, the compound is selected from the group consisting of a compound in Table 2

TABLE 2

| Compound No. | Structure |
|---|---|
| 001 | 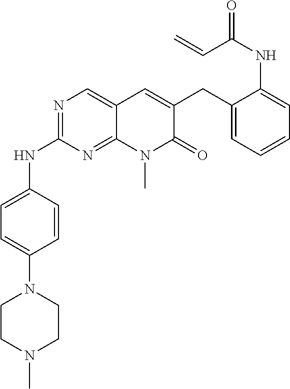 [SMILES: C=CC(=O)Nc1ccccc1Cc1cc2cnc(Nc3ccc(N4CCN(C)CC4)cc3)nc2n(C)c1=O] |
| 002 | 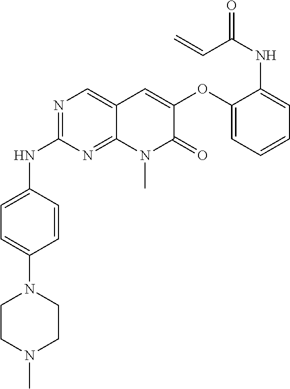 [SMILES: C=CC(=O)Nc1ccccc1Oc1cc2cnc(Nc3ccc(N4CCN(C)CC4)cc3)nc2n(C)c1=O] |
| 003 | 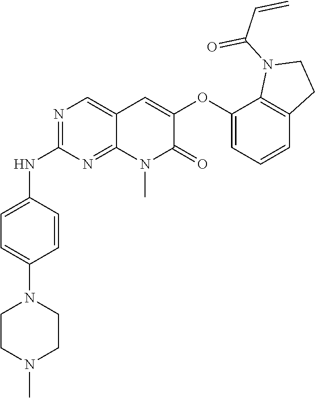 [SMILES: C=CC(=O)N1CCc2cccc(Oc3cc4cnc(Nc5ccc(N6CCN(C)CC6)cc5)nc4n(C)c3=O)c21] |
| 004 | 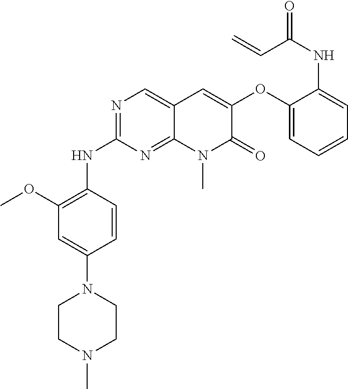 [SMILES: C=CC(=O)Nc1ccccc1Oc1cc2cnc(Nc3ccc(N4CCN(C)CC4)cc3OC)nc2n(C)c1=O] |
| 005 | 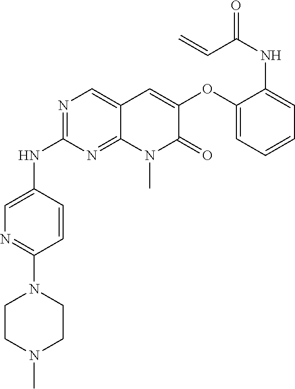 [SMILES: C=CC(=O)Nc1ccccc1Oc1cc2cnc(Nc3ccc(N4CCN(C)CC4)nc3)nc2n(C)c1=O] |
| 006 | 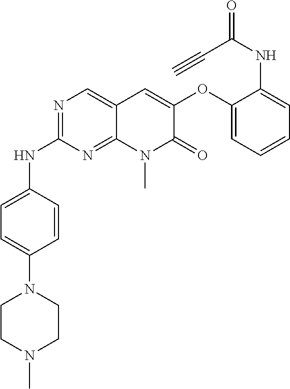 [SMILES: C#CC(=O)Nc1ccccc1Oc1cc2cnc(Nc3ccc(N4CCN(C)CC4)cc3)nc2n(C)c1=O] |
| 007 | 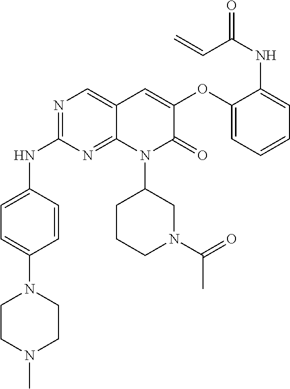 [SMILES: C=CC(=O)Nc1ccccc1Oc1cc2cnc(Nc3ccc(N4CCN(C)CC4)cc3)nc2n(C2CCCN(C(C)=O)C2)c1=O] |
| 008 | 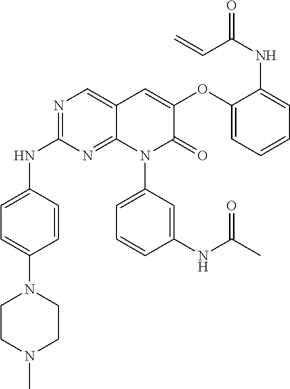 [SMILES: C=CC(=O)Nc1ccccc1Oc1cc2cnc(Nc3ccc(N4CCN(C)CC4)cc3)nc2n(-c2cccc(NC(C)=O)c2)c1=O] |
| 009 | 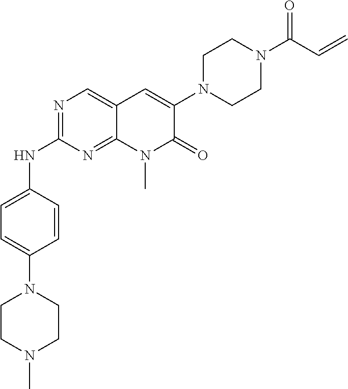 [SMILES: C=CC(=O)N1CCN(c2cc3cnc(Nc4ccc(N5CCN(C)CC5)cc4)nc3n(C)c2=O)CC1] |
| 010 | 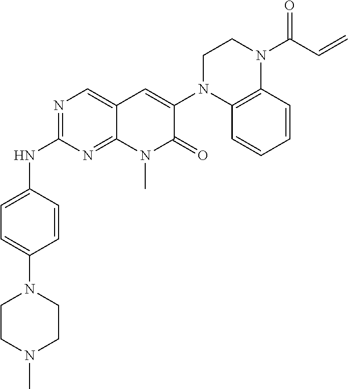 [SMILES: C=CC(=O)N1CCN(c2cc3cnc(Nc4ccc(N5CCN(C)CC5)cc4)nc3n(C)c2=O)c2ccccc21] |
| 011 | 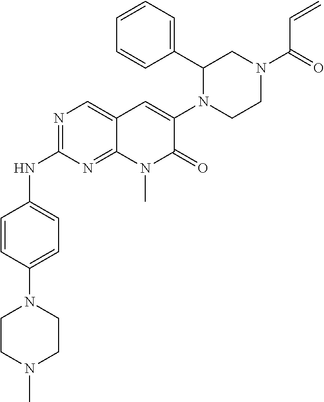 [SMILES: C=CC(=O)N1CCN(c2cc3cnc(Nc4ccc(N5CCN(C)CC5)cc4)nc3n(C)c2=O)C(c2ccccc2)C1] |
| 012 | 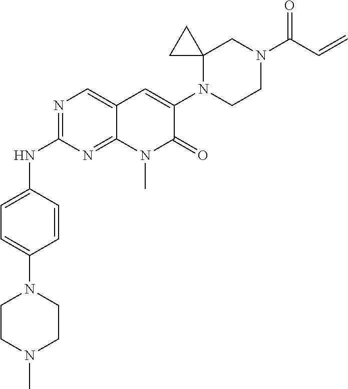 [SMILES: C=CC(=O)N1CCN(c2cc3cnc(Nc4ccc(N5CCN(C)CC5)cc4)nc3n(C)c2=O)C2(CC2)C1] |
| 013 | 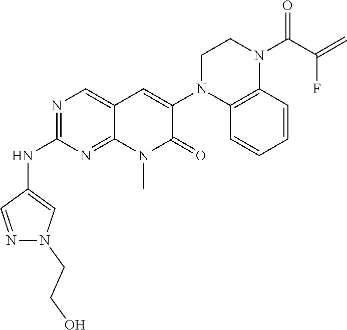 [SMILES: C=C(F)C(=O)N1CCN(c2cc3cnc(Nc4cnn(CCO)c4)nc3n(C)c2=O)c2ccccc21] |
| 014 | 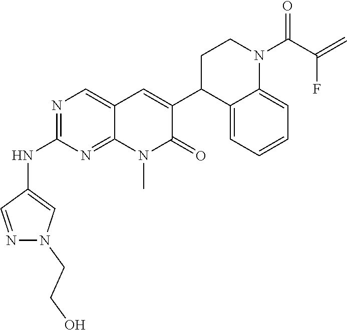 [SMILES: C=C(F)C(=O)N1CCC(c2cc3cnc(Nc4cnn(CCO)c4)nc3n(C)c2=O)c2ccccc21] |
| 015 | 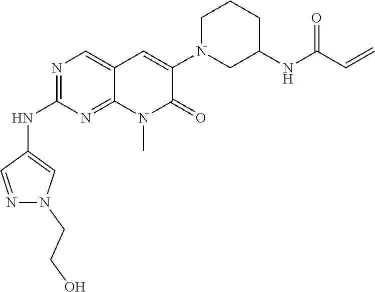 [SMILES: C=CC(=O)NC1CCCN(c2cc3cnc(Nc4cnn(CCO)c4)nc3n(C)c2=O)C1] |
| 016 | 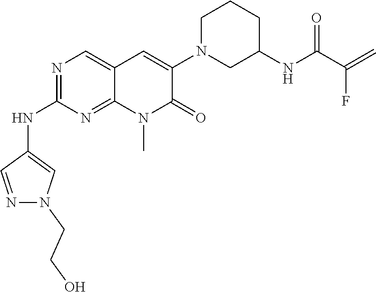 [SMILES: C=C(F)C(=O)NC1CCCN(c2cc3cnc(Nc4cnn(CCO)c4)nc3n(C)c2=O)C1] |
| 017 | 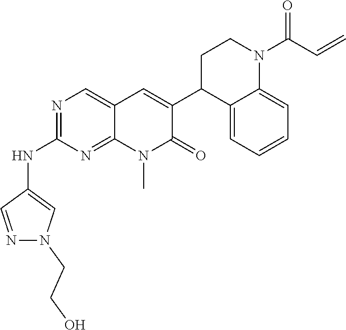 [SMILES: C=CC(=O)N1CCC(c2cc3cnc(Nc4cnn(CCO)c4)nc3n(C)c2=O)c2ccccc21] |
| 018 | 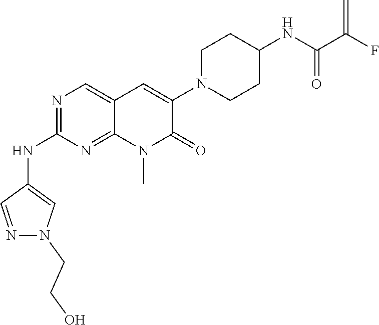 [SMILES: C=C(F)C(=O)NC1CCN(c2cc3cnc(Nc4cnn(CCO)c4)nc3n(C)c2=O)CC1] |
| 019 | 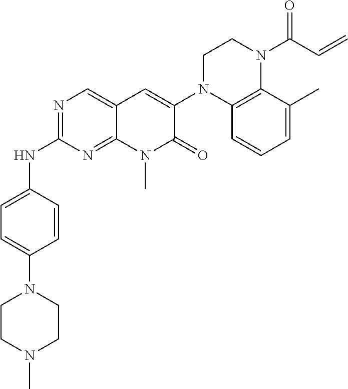 [SMILES: C=CC(=O)N1CCN(c2cc3cnc(Nc4ccc(N5CCN(C)CC5)cc4)nc3n(C)c2=O)c2cccc(C)c21] |
| 020 | 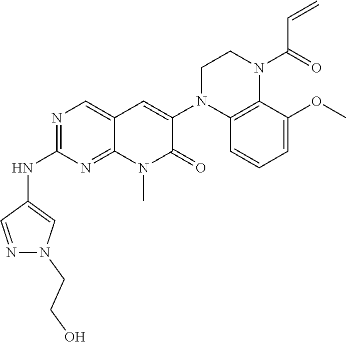 [SMILES: C=CC(=O)N1CCN(c2cc3cnc(Nc4cnn(CCO)c4)nc3n(C)c2=O)c2cccc(OC)c21] |
| 021 | 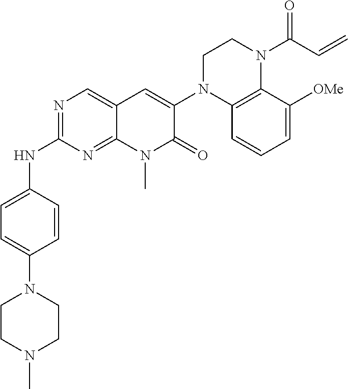 [SMILES: C=CC(=O)N1CCN(c2cc3cnc(Nc4ccc(N5CCN(C)CC5)cc4)nc3n(C)c2=O)c2cccc(OC)c21] |
| 022 | 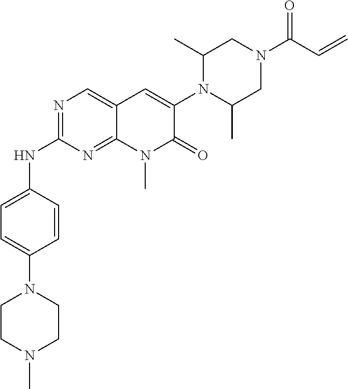 [SMILES: C=CC(=O)N1CC(C)N(c2cc3cnc(Nc4ccc(N5CCN(C)CC5)cc4)nc3n(C)c2=O)C(C)C1] |
| 023 | 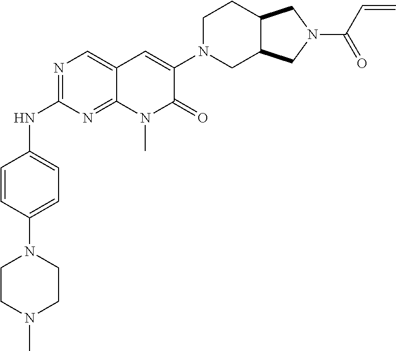 |
| 024 | 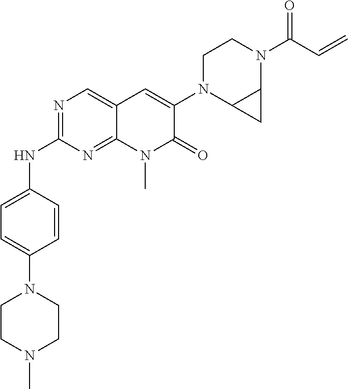 [SMILES: C=CC(=O)N1CCN(c2cc3cnc(Nc4ccc(N5CCN(C)CC5)cc4)nc3n(C)c2=O)C2CC21] |
| 025 | 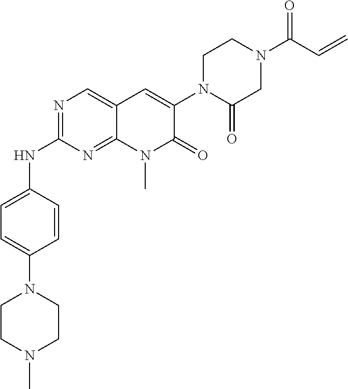 [SMILES: C=CC(=O)N1CCN(c2cc3cnc(Nc4ccc(N5CCN(C)CC5)cc4)nc3n(C)c2=O)C(=O)C1] |
| 026 | 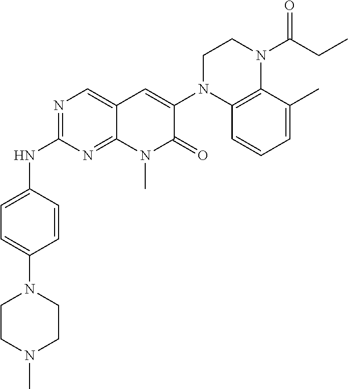 [SMILES: CCC(=O)N1CCN(c2cc3cnc(Nc4ccc(N5CCN(C)CC5)cc4)nc3n(C)c2=O)c2cccc(C)c21] |
| 027 | 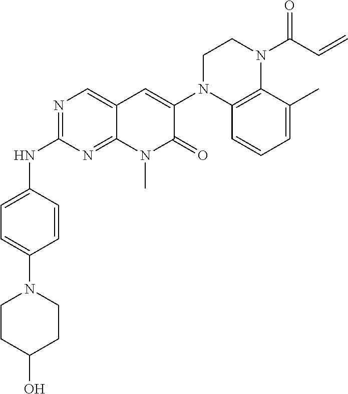 [SMILES: C=CC(=O)N1CCN(c2cc3cnc(Nc4ccc(N5CCC(O)CC5)cc4)nc3n(C)c2=O)c2cccc(C)c21] |
| 028 | 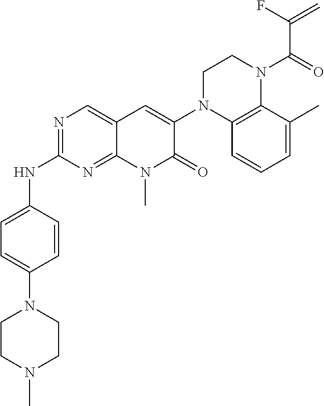 [SMILES: C=C(F)C(=O)N1CCN(c2cc3cnc(Nc4ccc(N5CCN(C)CC5)cc4)nc3n(C)c2=O)c2cccc(C)c21] |
| 029 | 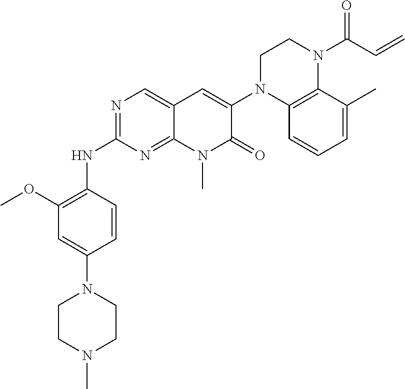 [SMILES: C=CC(=O)N1CCN(c2cc3cnc(Nc4ccc(N5CCN(C)CC5)cc4OC)nc3n(C)c2=O)c2cccc(C)c21] |
| 030 | 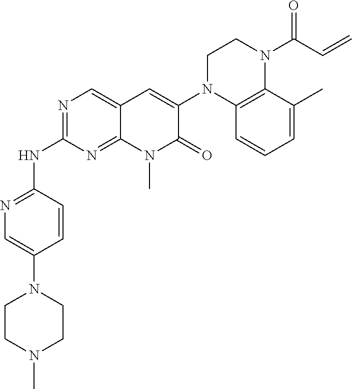 [SMILES: C=CC(=O)N1CCN(c2cc3cnc(Nc4ccc(N5CCN(C)CC5)cn4)nc3n(C)c2=O)c2cccc(C)c21] |
| 031 | 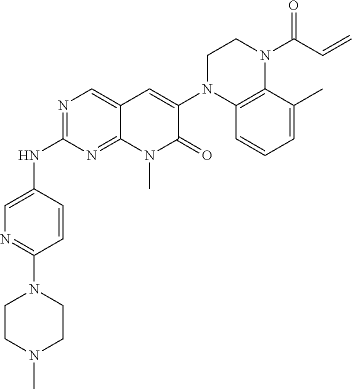 [SMILES: C=CC(=O)N1CCN(c2cc3cnc(Nc4ccc(N5CCN(C)CC5)nc4)nc3n(C)c2=O)c2cccc(C)c21] |
| 032 | 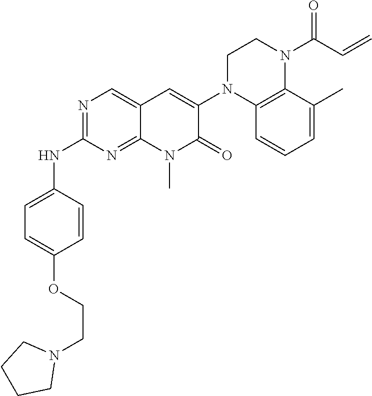 [SMILES: C=CC(=O)N1CCN(c2cc3cnc(Nc4ccc(OCCN5CCCC5)cc4)nc3n(C)c2=O)c2cccc(C)c21] |
| 033 | 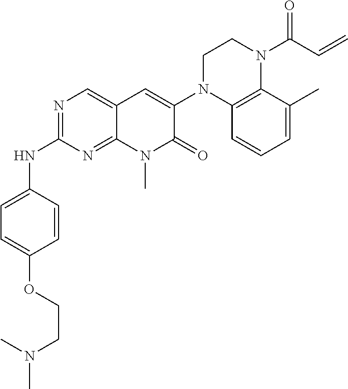 [SMILES: C=CC(=O)N1CCN(c2cc3cnc(Nc4ccc(OCCN(C)C)cc4)nc3n(C)c2=O)c2cccc(C)c21] |
| 034 | 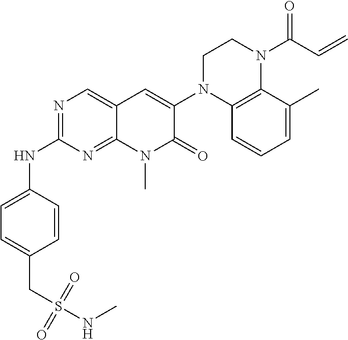 [SMILES: C=CC(=O)N1CCN(c2cc3cnc(Nc4ccc(CS(=O)(=O)NC)cc4)nc3n(C)c2=O)c2cccc(C)c21] |
| 035 | 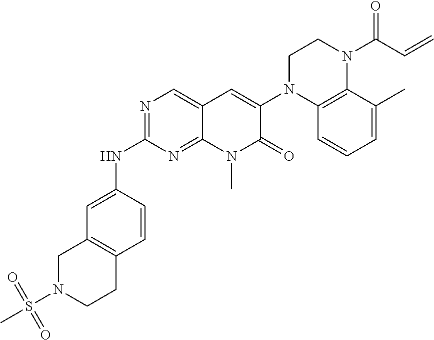 [SMILES: C=CC(=O)N1CCN(c2cc3cnc(Nc4ccc5c(c4)CN(S(C)(=O)=O)CC5)nc3n(C)c2=O)c2cccc(C)c21] |
| 036 | 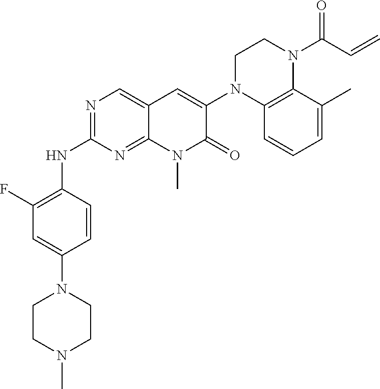 [SMILES: C=CC(=O)N1CCN(c2cc3cnc(Nc4ccc(N5CCN(C)CC5)cc4F)nc3n(C)c2=O)c2cccc(C)c21] |
| 037 | 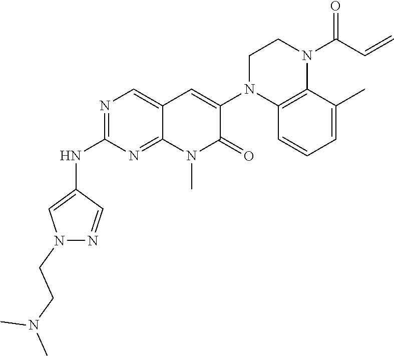 [SMILES: C=CC(=O)N1CCN(c2cc3cnc(Nc4cnn(CCN(C)C)c4)nc3n(C)c2=O)c2cccc(C)c21] |
| 038 | 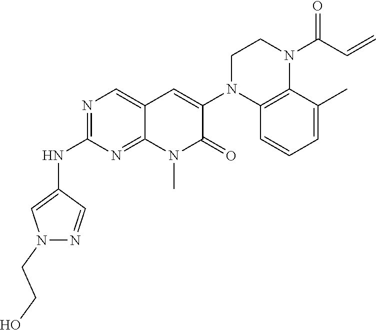 [SMILES: C=CC(=O)N1CCN(c2cc3cnc(Nc4cnn(CCO)c4)nc3n(C)c2=O)c2cccc(C)c21] |
| 039 | 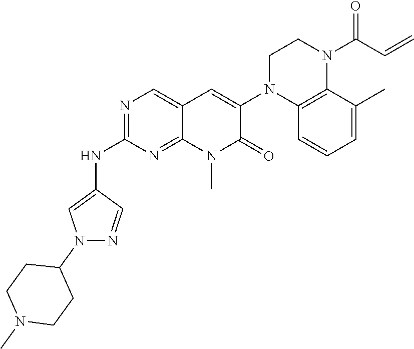 [SMILES: C=CC(=O)N1CCN(c2cc3cnc(Nc4cnn(C5CCN(C)CC5)c4)nc3n(C)c2=O)c2cccc(C)c21] |
| 040 | 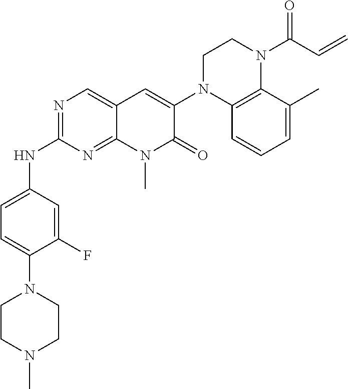 [SMILES: C=CC(=O)N1CCN(c2cc3cnc(Nc4ccc(N5CCN(C)CC5)c(F)c4)nc3n(C)c2=O)c2cccc(C)c21] |
| 041 | 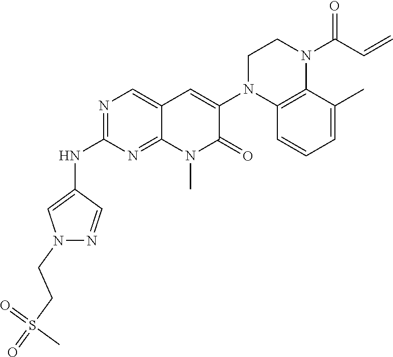 [SMILES: C=CC(=O)N1CCN(c2cc3cnc(Nc4cnn(CCS(C)(=O)=O)c4)nc3n(C)c2=O)c2cccc(C)c21] |
| 042 | 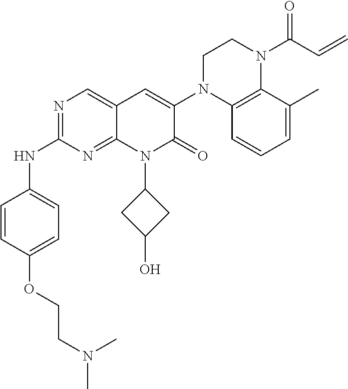 [SMILES: C=CC(=O)N1CCN(c2cc3cnc(Nc4ccc(OCCN(C)C)cc4)nc3n(C3CC(O)C3)c2=O)c2cccc(C)c21] |
| 043 | 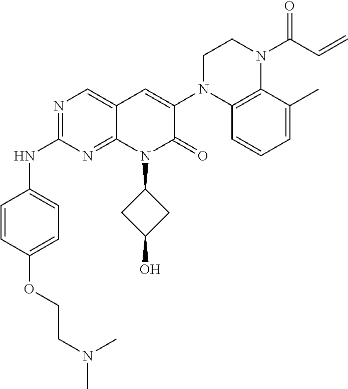 [SMILES: C=CC(=O)N1CCN(c2cc3cnc(Nc4ccc(OCCN(C)C)cc4)nc3n([C@H]3C[C@@H](O)C3)c2=O)c2cccc(C)c21] |
| 044 | 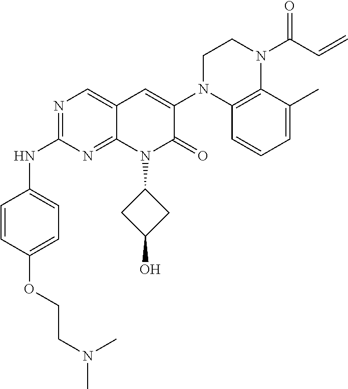 [SMILES: C=CC(=O)N1CCN(c2cc3cnc(Nc4ccc(OCCN(C)C)cc4)nc3n([C@H]3C[C@H](O)C3)c2=O)c2cccc(C)c21] |
| 045 | 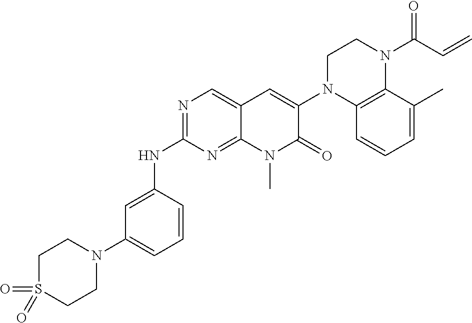 [SMILES: C=CC(=O)N1CCN(c2cc3cnc(Nc4cccc(N5CCS(=O)(=O)CC5)c4)nc3n(C)c2=O)c2cccc(C)c21] |
| 046 | 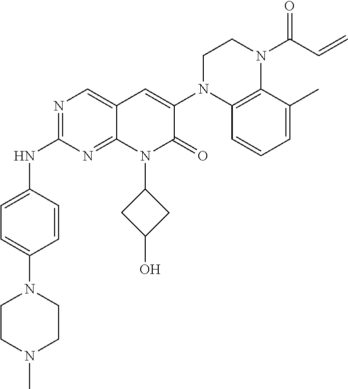 [SMILES: C=CC(=O)N1CCN(c2cc3cnc(Nc4ccc(N5CCN(C)CC5)cc4)nc3n(C3CC(O)C3)c2=O)c2cccc(C)c21] |
| 047 | 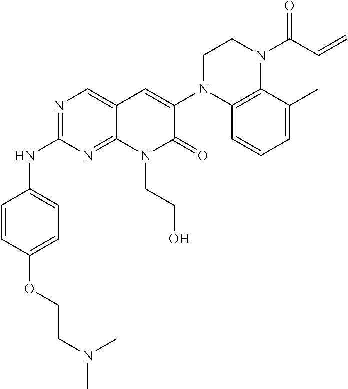 [SMILES: C=CC(=O)N1CCN(c2cc3cnc(Nc4ccc(OCCN(C)C)cc4)nc3n(CCO)c2=O)c2cccc(C)c21] |
| 048 | 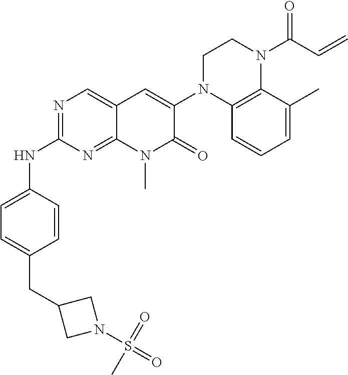 [SMILES: C=CC(=O)N1CCN(c2cc3cnc(Nc4ccc(CC5CN(S(C)(=O)=O)C5)cc4)nc3n(C)c2=O)c2cccc(C)c21] |
| 049 | 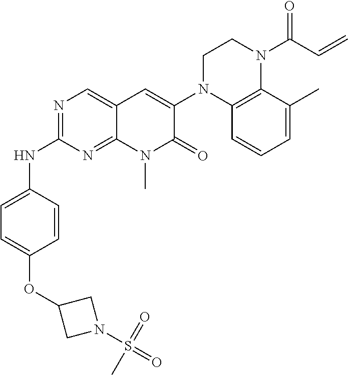 [SMILES: C=CC(=O)N1CCN(c2cc3cnc(Nc4ccc(OC5CN(S(C)(=O)=O)C5)cc4)nc3n(C)c2=O)c2cccc(C)c21] |
| 050 | 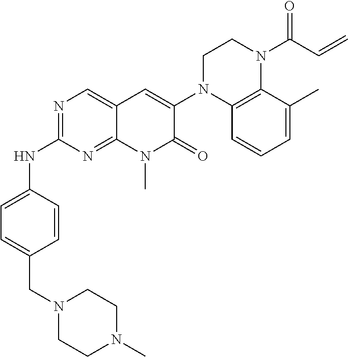 [SMILES: C=CC(=O)N1CCN(c2cc3cnc(Nc4ccc(CN5CCN(C)CC5)cc4)nc3n(C)c2=O)c2cccc(C)c21] |
| 051 | 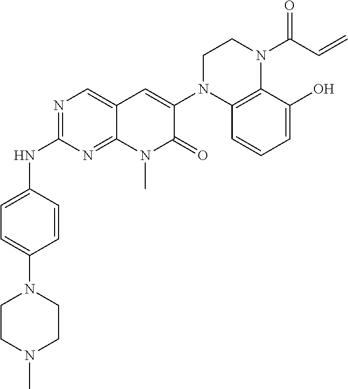 [SMILES: C=CC(=O)N1CCN(c2cc3cnc(Nc4ccc(N5CCN(C)CC5)cc4)nc3n(C)c2=O)c2cccc(O)c21] |
| 052 | 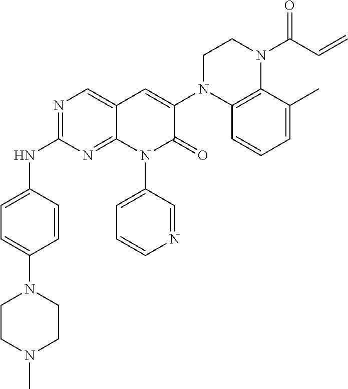 [SMILES: C=CC(=O)N1CCN(c2cc3cnc(Nc4ccc(N5CCN(C)CC5)cc4)nc3n(-c3cccnc3)c2=O)c2cccc(C)c21] |
| 053 | 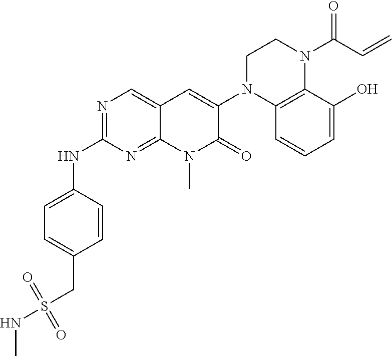 [SMILES: C=CC(=O)N1CCN(c2cc3cnc(Nc4ccc(CS(=O)(=O)NC)cc4)nc3n(C)c2=O)c2cccc(O)c21] |
| 054 | 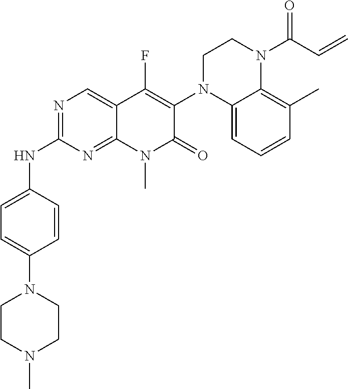 [SMILES: C=CC(=O)N1CCN(c2c(F)c3cnc(Nc4ccc(N5CCN(C)CC5)cc4)nc3n(C)c2=O)c2cccc(C)c21] |
| 055 | 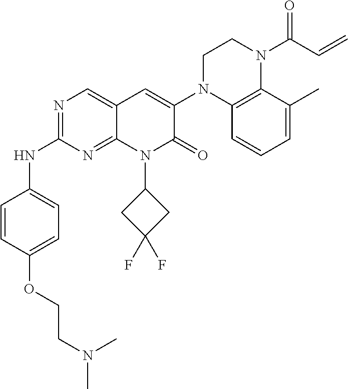 [SMILES: C=CC(=O)N1CCN(c2cc3cnc(Nc4ccc(OCCN(C)C)cc4)nc3n(C3CC(F)(F)C3)c2=O)c2cccc(C)c21] |
| 056 | 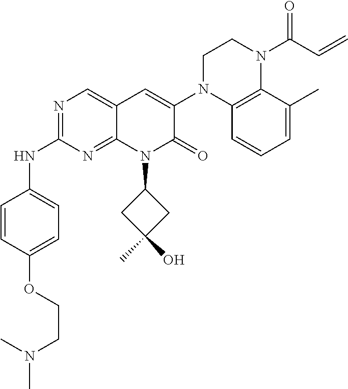 [SMILES: C=CC(=O)N1CCN(c2cc3cnc(Nc4ccc(OCCN(C)C)cc4)nc3n([C@H]3C[C@@](C)(O)C3)c2=O)c2cccc(C)c21] |
| 057 | 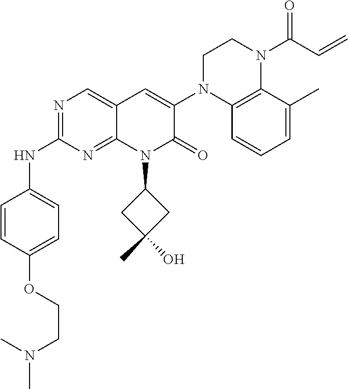 [SMILES: C=CC(=O)N1CCN(c2cc3cnc(Nc4ccc(OCCN(C)C)cc4)nc3n([C@H]3C[C@](C)(O)C3)c2=O)c2cccc(C)c21] |
| 058 | 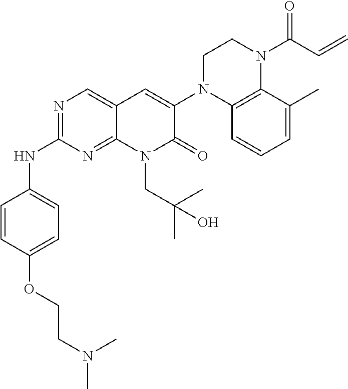 [SMILES: C=CC(=O)N1CCN(c2cc3cnc(Nc4ccc(OCCN(C)C)cc4)nc3n(CC(C)(C)O)c2=O)c2cccc(C)c21] |
| 059 | 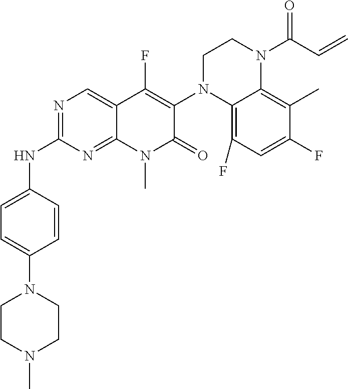 [SMILES: C=CC(=O)N1CCN(c2c(F)c3cnc(Nc4ccc(N5CCN(C)CC5)cc4)nc3n(C)c2=O)c2c(F)cc(F)c(C)c21] |
| 060 | 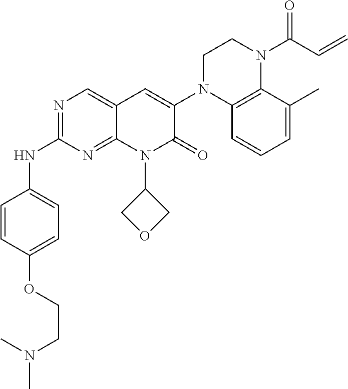 [SMILES: C=CC(=O)N1CCN(c2cc3cnc(Nc4ccc(OCCN(C)C)cc4)nc3n(C3COC3)c2=O)c2cccc(C)c21] |
| 061 | 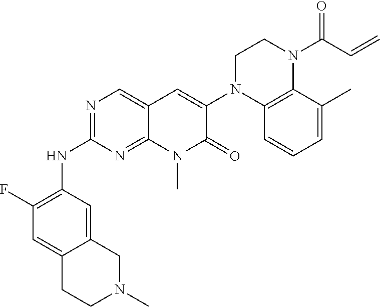 [SMILES: C=CC(=O)N1CCN(c2cc3cnc(Nc4cc5c(cc4F)CCN(C)C5)nc3n(C)c2=O)c2cccc(C)c21] |
| 062 | 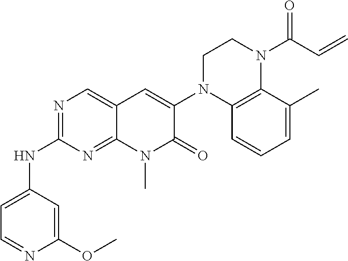 [SMILES: C=CC(=O)N1CCN(c2cc3cnc(Nc4ccnc(OC)c4)nc3n(C)c2=O)c2cccc(C)c21] |
| 063 | 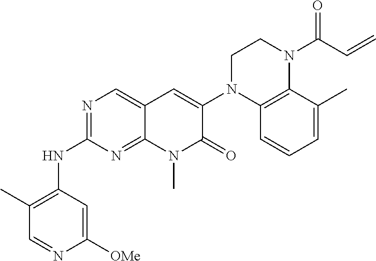 [SMILES: C=CC(=O)N1CCN(c2cc3cnc(Nc4cc(OC)ncc4C)nc3n(C)c2=O)c2cccc(C)c21] |
| 064 | 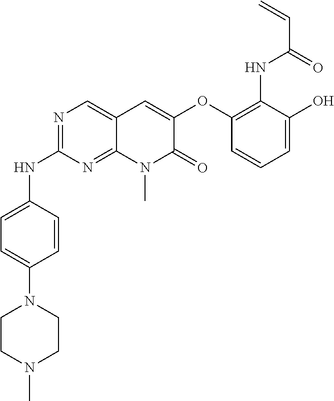 [SMILES: C=CC(=O)Nc1c(O)cccc1Oc1cc2cnc(Nc3ccc(N4CCN(C)CC4)cc3)nc2n(C)c1=O] |
| 065 | 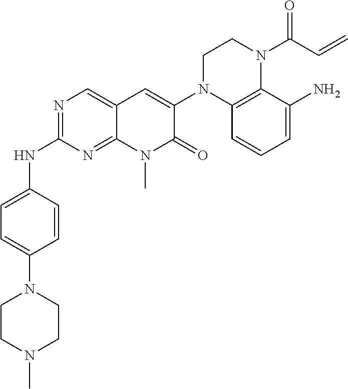 [SMILES: C=CC(=O)N1CCN(c2cc3cnc(Nc4ccc(N5CCN(C)CC5)cc4)nc3n(C)c2=O)c2cccc(N)c21] |
| 066 | 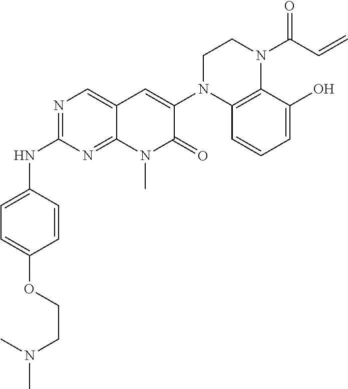 [SMILES: C=CC(=O)N1CCN(c2cc3cnc(Nc4ccc(OCCN(C)C)cc4)nc3n(C)c2=O)c2cccc(O)c21] |
| 067 | 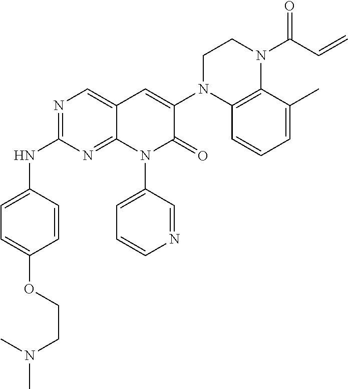 [SMILES: C=CC(=O)N1CCN(c2cc3cnc(Nc4ccc(OCCN(C)C)cc4)nc3n(-c3cccnc3)c2=O)c2cccc(C)c21] |
| 068 | 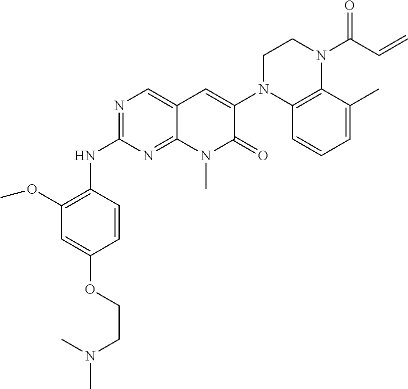 [SMILES: C=CC(=O)N1CCN(c2cc3cnc(Nc4ccc(OCCN(C)C)cc4OC)nc3n(C)c2=O)c2cccc(C)c21] |
| 069 | 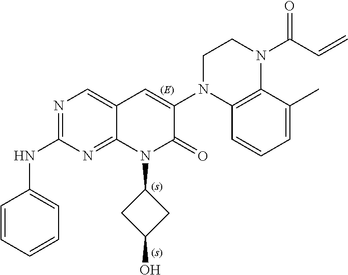 [SMILES: C=CC(=O)N1CCN(c2cc3cnc(Nc4ccccc4)nc3n([C@H]3C[C@@H](O)C3)c2=O)c2cccc(C)c21] |
| 070 | 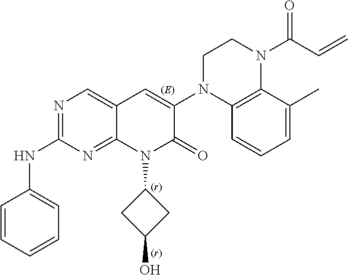 [SMILES: C=CC(=O)N1CCN(c2cc3cnc(Nc4ccccc4)nc3n([C@H]3C[C@H](O)C3)c2=O)c2cccc(C)c21] |
| 071 | 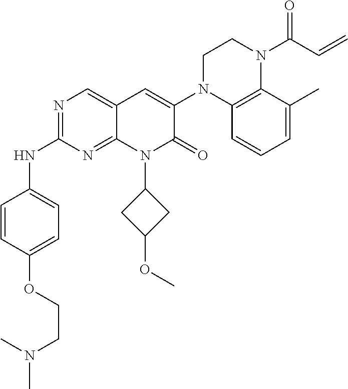 [SMILES: C=CC(=O)N1CCN(c2cc3cnc(Nc4ccc(OCCN(C)C)cc4)nc3n(C3CC(OC)C3)c2=O)c2cccc(C)c21] |
| 072 | 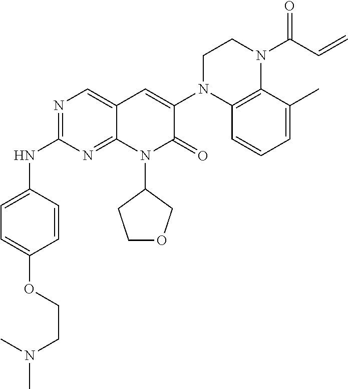 [SMILES: C=CC(=O)N1CCN(c2cc3cnc(Nc4ccc(OCCN(C)C)cc4)nc3n(C3CCOC3)c2=O)c2cccc(C)c21] |
| 073 | 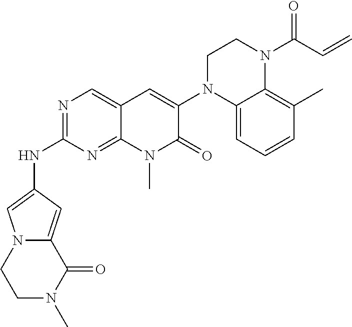 [SMILES: C=CC(=O)N1CCN(c2cc3cnc(Nc4cc5n(c4)CCN(C)C5=O)nc3n(C)c2=O)c2cccc(C)c21] |
| 074 | 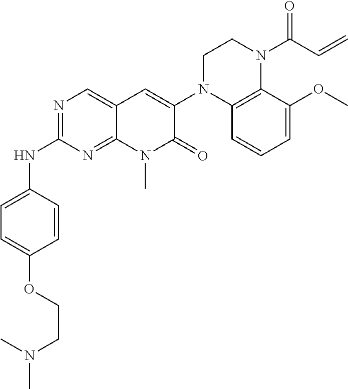 [SMILES: C=CC(=O)N1CCN(c2cc3cnc(Nc4ccc(OCCN(C)C)cc4)nc3n(C)c2=O)c2cccc(OC)c21] |
| 075 | 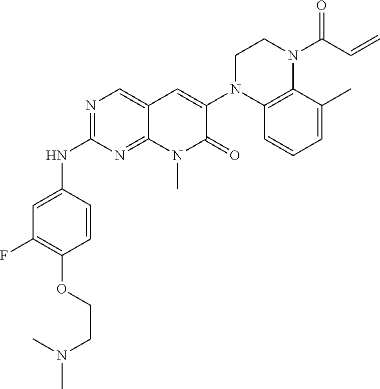 [SMILES: C=CC(=O)N1CCN(c2cc3cnc(Nc4ccc(OCCN(C)C)c(F)c4)nc3n(C)c2=O)c2cccc(C)c21] |
| 076 | 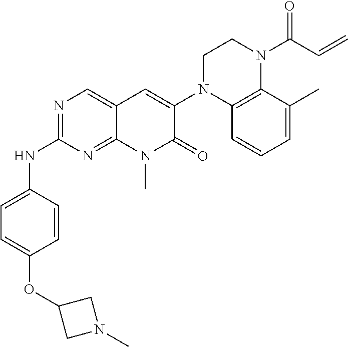 [SMILES: C=CC(=O)N1CCN(c2cc3cnc(Nc4ccc(OC5CN(C)C5)cc4)nc3n(C)c2=O)c2cccc(C)c21] |
| 077 | 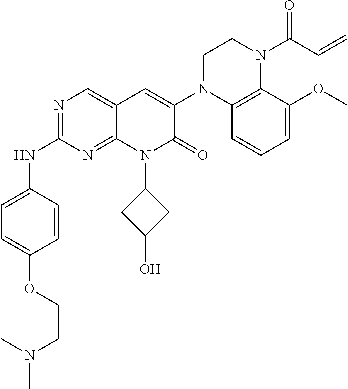 [SMILES: C=CC(=O)N1CCN(c2cc3cnc(Nc4ccc(OCCN(C)C)cc4)nc3n(C3CC(O)C3)c2=O)c2cccc(OC)c21] |
| 078 | 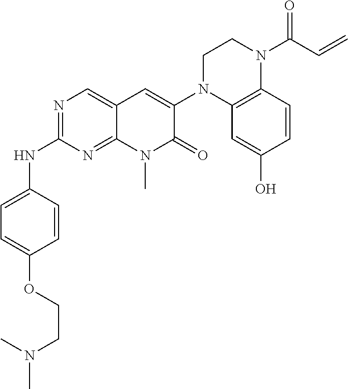 [SMILES: C=CC(=O)N1CCN(c2cc3cnc(Nc4ccc(OCCN(C)C)cc4)nc3n(C)c2=O)c2cc(O)ccc21] |
| 079 | 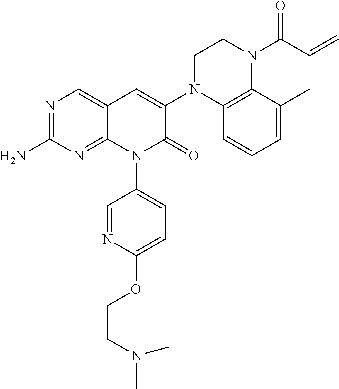 [SMILES: C=CC(=O)N1CCN(c2cc3cnc(N)nc3n(-c3ccc(OCCN(C)C)nc3)c2=O)c2cccc(C)c21] |
| 080 | 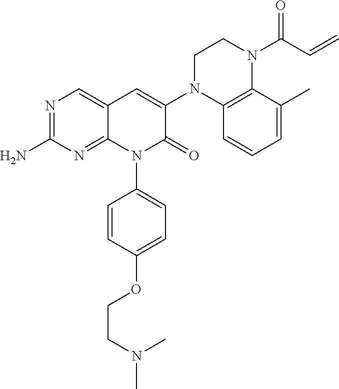 [SMILES: C=CC(=O)N1CCN(c2cc3cnc(N)nc3n(-c3ccc(OCCN(C)C)cc3)c2=O)c2cccc(C)c21] |
| 081 | 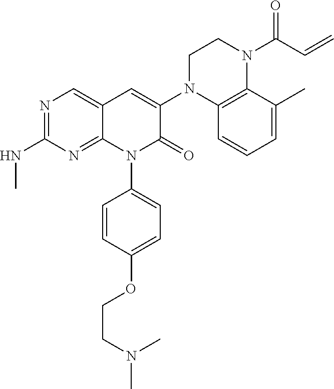 [SMILES: C=CC(=O)N1CCN(c2cc3cnc(NC)nc3n(-c3ccc(OCCN(C)C)cc3)c2=O)c2cccc(C)c21] |
| 082 | 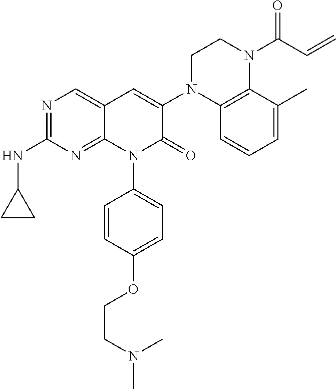 [SMILES: C=CC(=O)N1CCN(c2cc3cnc(NC4CC4)nc3n(-c3ccc(OCCN(C)C)cc3)c2=O)c2cccc(C)c21] |
| 083 | 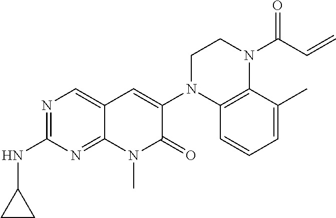 [SMILES: C=CC(=O)N1CCN(c2cc3cnc(NC4CC4)nc3n(C)c2=O)c2cccc(C)c21] |
| 084 | 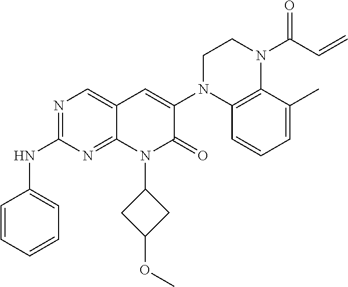 [SMILES: C=CC(=O)N1CCN(c2cc3cnc(Nc4ccccc4)nc3n(C3CC(OC)C3)c2=O)c2cccc(C)c21] |
| 085 | 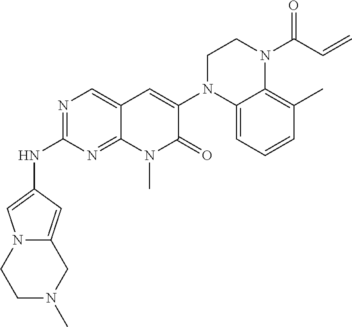 [SMILES: C=CC(=O)N1CCN(c2cc3cnc(Nc4cc5n(c4)CCN(C)C5)nc3n(C)c2=O)c2cccc(C)c21] |
| 086 | 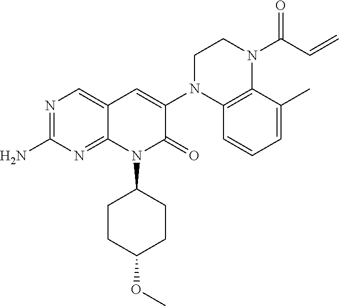 [SMILES: C=CC(=O)N1CCN(c2cc3cnc(N)nc3n([C@H]3CC[C@H](OC)CC3)c2=O)c2cccc(C)c21] |
| 087 | 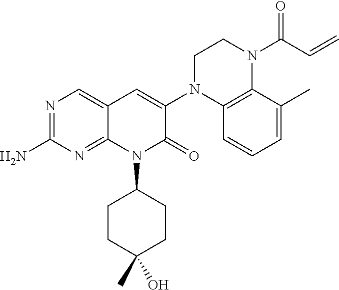 [SMILES: C=CC(=O)N1CCN(c2cc3cnc(N)nc3n([C@H]3CC[C@](C)(O)CC3)c2=O)c2cccc(C)c21] |
| 088 | 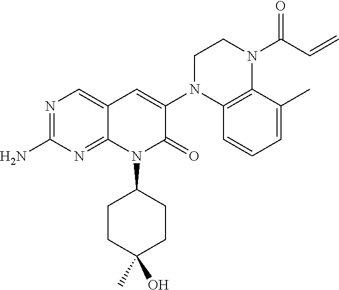 [SMILES: C=CC(=O)N1CCN(c2cc3cnc(N)nc3n([C@H]3CC[C@@](C)(O)CC3)c2=O)c2cccc(C)c21] |
| 089 | 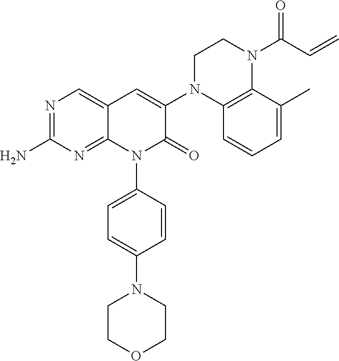 [SMILES: C=CC(=O)N1CCN(c2cc3cnc(N)nc3n(-c3ccc(N4CCOCC4)cc3)c2=O)c2cccc(C)c21] |
| 090 | 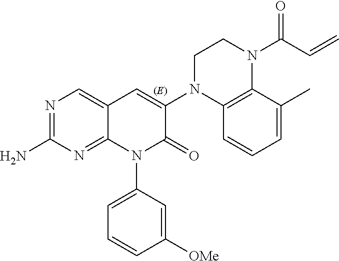 [SMILES: C=CC(=O)N1CCN(c2cc3cnc(N)nc3n(-c3cccc(OC)c3)c2=O)c2cccc(C)c21] |
| 091 | 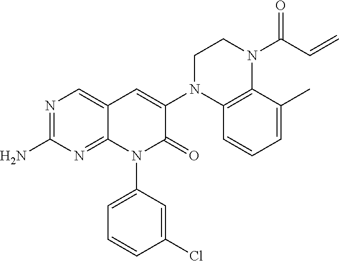 [SMILES: C=CC(=O)N1CCN(c2cc3cnc(N)nc3n(-c3cccc(Cl)c3)c2=O)c2cccc(C)c21] |
| 092 | 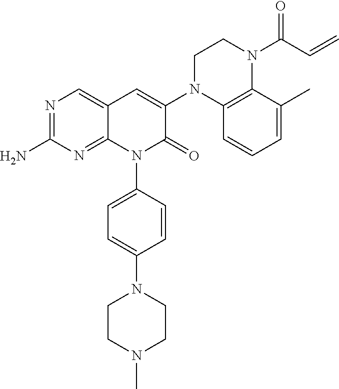 [SMILES: C=CC(=O)N1CCN(c2cc3cnc(N)nc3n(-c3ccc(N4CCN(C)CC4)cc3)c2=O)c2cccc(C)c21] |
| 093 | 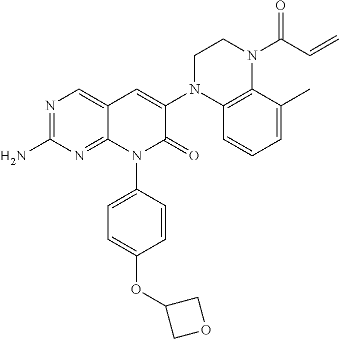 [SMILES: C=CC(=O)N1CCN(c2cc3cnc(N)nc3n(-c3ccc(OC4COC4)cc3)c2=O)c2cccc(C)c21] |
| 094 | 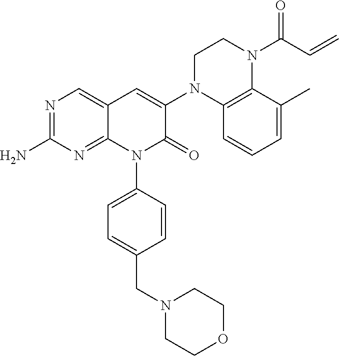 [SMILES: C=CC(=O)N1CCN(c2cc3cnc(N)nc3n(-c3ccc(CN4CCOCC4)cc3)c2=O)c2cccc(C)c21] |
| 095 | 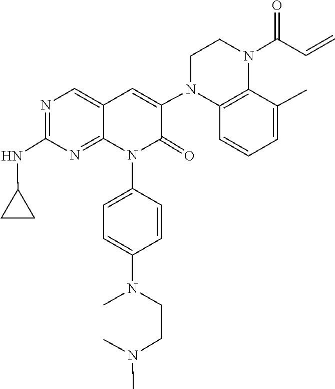 [SMILES: C=CC(=O)N1CCN(c2cc3cnc(NC4CC4)nc3n(-c3ccc(N(C)CCN(C)C)cc3)c2=O)c2cccc(C)c21] |
| 096 | 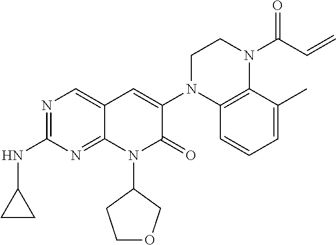 [SMILES: C=CC(=O)N1CCN(c2cc3cnc(NC4CC4)nc3n(C3CCOC3)c2=O)c2cccc(C)c21] |
| 097 | 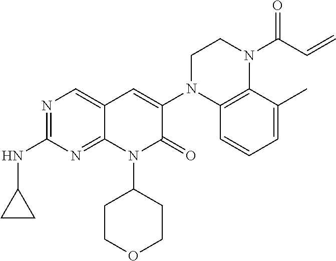 [SMILES: C=CC(=O)N1CCN(c2cc3cnc(NC4CC4)nc3n(C3CCOCC3)c2=O)c2cccc(C)c21] |
| 098 | 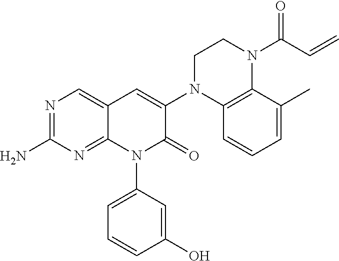 [SMILES: C=CC(=O)N1CCN(c2cc3cnc(N)nc3n(-c3cccc(O)c3)c2=O)c2cccc(C)c21] |
| 099 | 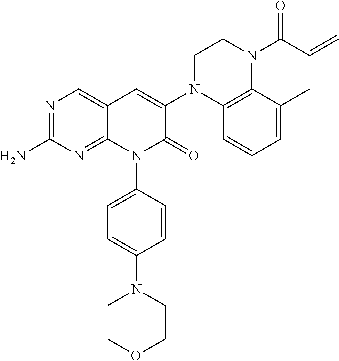 [SMILES: C=CC(=O)N1CCN(c2cc3cnc(N)nc3n(-c3ccc(N(C)CCOC)cc3)c2=O)c2cccc(C)c21] |
| 100 | 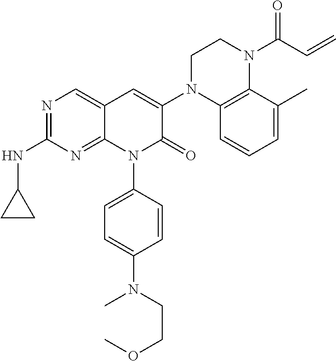 [SMILES: C=CC(=O)N1CCN(c2cc3cnc(NC4CC4)nc3n(-c3ccc(N(C)CCOC)cc3)c2=O)c2cccc(C)c21] |
| 101 | 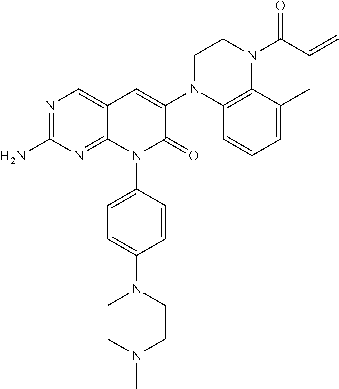 [SMILES: C=CC(=O)N1CCN(c2cc3cnc(N)nc3n(-c3ccc(N(C)CCN(C)C)cc3)c2=O)c2cccc(C)c21] |
| 102 | 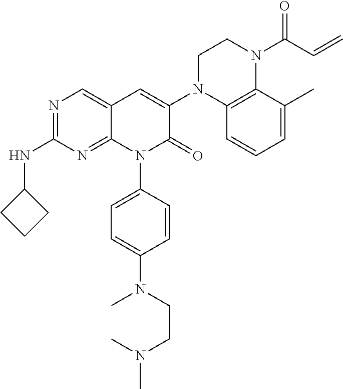 [SMILES: C=CC(=O)N1CCN(c2cc3cnc(NC4CCC4)nc3n(-c3ccc(N(C)CCN(C)C)cc3)c2=O)c2cccc(C)c21] |
| 103 | 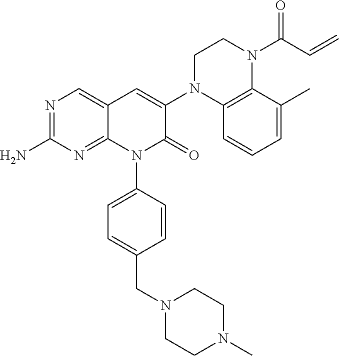 [SMILES: C=CC(=O)N1CCN(c2cc3cnc(N)nc3n(-c3ccc(CN4CCN(C)CC4)cc3)c2=O)c2cccc(C)c21] |
| 104 | 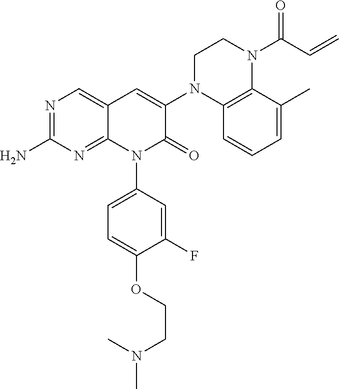 [SMILES: C=CC(=O)N1CCN(c2cc3cnc(N)nc3n(-c3ccc(OCCN(C)C)c(F)c3)c2=O)c2cccc(C)c21] |
| 105 | 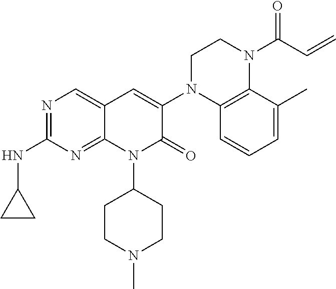 [SMILES: C=CC(=O)N1CCN(c2cc3cnc(NC4CC4)nc3n(C3CCN(C)CC3)c2=O)c2cccc(C)c21] |
| 106 | 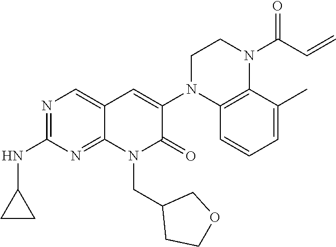 [SMILES: C=CC(=O)N1CCN(c2cc3cnc(NC4CC4)nc3n(CC3CCOC3)c2=O)c2cccc(C)c21] |
| 107 | 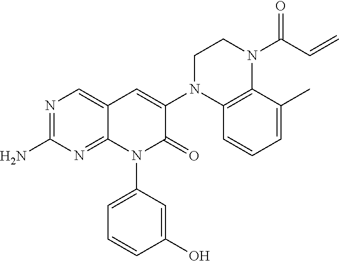 [SMILES: C=CC(=O)N1CCN(c2cc3cnc(N)nc3n(-c3cccc(O)c3)c2=O)c2cccc(C)c21] |
| 108 | 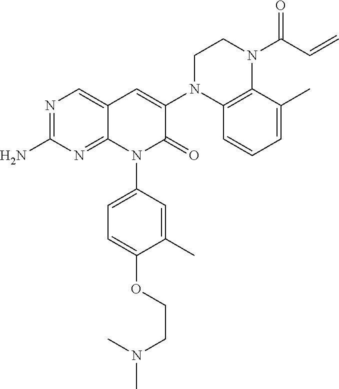 [SMILES: C=CC(=O)N1CCN(c2cc3cnc(N)nc3n(-c3ccc(OCCN(C)C)c(C)c3)c2=O)c2cccc(C)c21] |
| 109 | 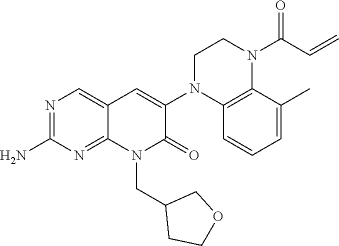 [SMILES: C=CC(=O)N1CCN(c2cc3cnc(N)nc3n(CC3CCOC3)c2=O)c2cccc(C)c21] |
| 110 | 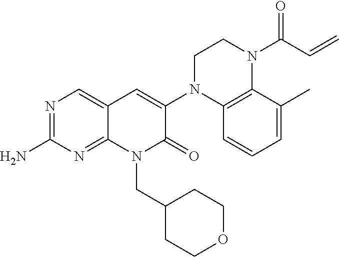 [SMILES: C=CC(=O)N1CCN(c2cc3cnc(N)nc3n(CC3CCOCC3)c2=O)c2cccc(C)c21] |
| 111 | 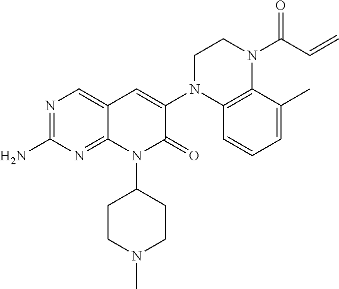 [SMILES: C=CC(=O)N1CCN(c2cc3cnc(N)nc3n(C3CCN(C)CC3)c2=O)c2cccc(C)c21] |
| 112 | 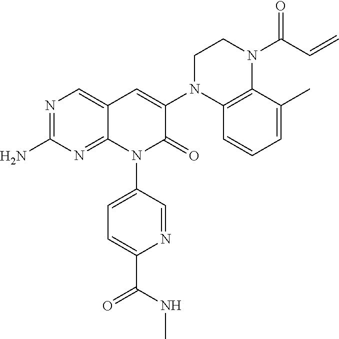 [SMILES: C=CC(=O)N1CCN(c2cc3cnc(N)nc3n(-c3ccc(C(=O)NC)nc3)c2=O)c2cccc(C)c21] |
| 113 | 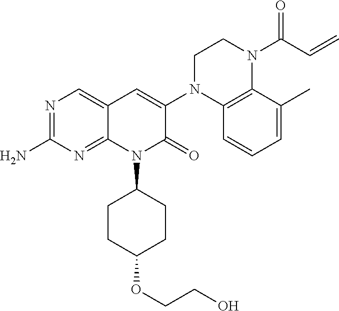 [SMILES: C=CC(=O)N1CCN(c2cc3cnc(N)nc3n([C@H]3CC[C@H](OCCO)CC3)c2=O)c2cccc(C)c21] |
| 114 | 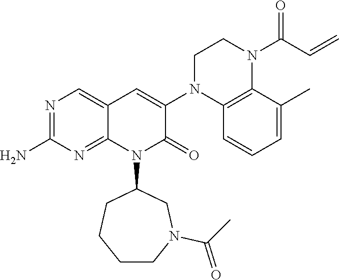 [SMILES: C=CC(=O)N1CCN(c2cc3cnc(N)nc3n([C@@H]3CCCCN(C(C)=O)C3)c2=O)c2cccc(C)c21] |
| 115 | 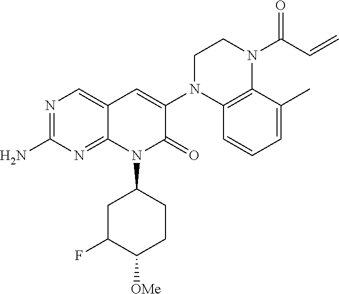 [SMILES: C=CC(=O)N1CCN(c2cc3cnc(N)nc3n([C@H]3CC[C@H](OC)C(F)C3)c2=O)c2cccc(C)c21] |
| 116 | 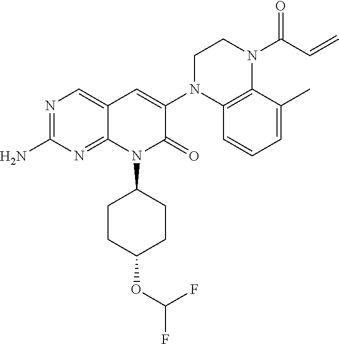 [SMILES: C=CC(=O)N1CCN(c2cc3cnc(N)nc3n([C@H]3CC[C@H](OC(F)F)CC3)c2=O)c2cccc(C)c21] |
| 117 | 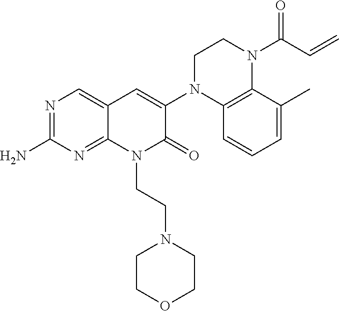 [SMILES: C=CC(=O)N1CCN(c2cc3cnc(N)nc3n(CCN3CCOCC3)c2=O)c2cccc(C)c21] |
| 118 | 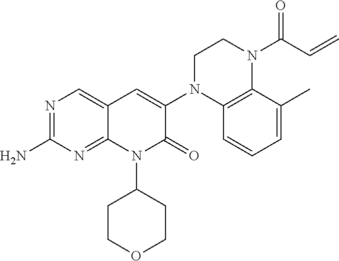 [SMILES: C=CC(=O)N1CCN(c2cc3cnc(N)nc3n(C3CCOCC3)c2=O)c2cccc(C)c21] |
| 119 | 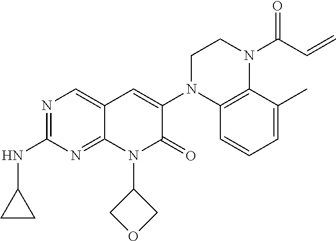 [SMILES: C=CC(=O)N1CCN(c2cc3cnc(NC4CC4)nc3n(C3COC3)c2=O)c2cccc(C)c21] |
| 120 | 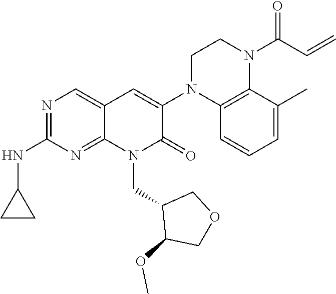 [SMILES: C=CC(=O)N1CCN(c2cc3cnc(NC4CC4)nc3n(C[C@@H]3COC[C@H]3OC)c2=O)c2cccc(C)c21] |
| 121 | 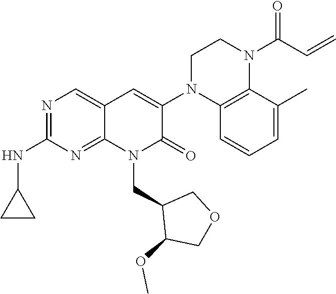 [SMILES: C=CC(=O)N1CCN(c2cc3cnc(NC4CC4)nc3n(C[C@H]3COC[C@H]3OC)c2=O)c2cccc(C)c21] |
| 122 | 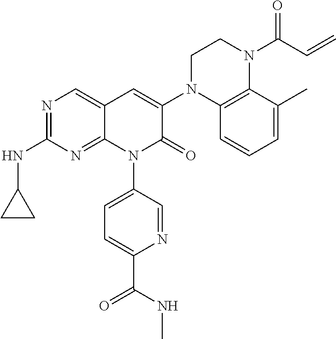 [SMILES: C=CC(=O)N1CCN(c2cc3cnc(NC4CC4)nc3n(-c3ccc(C(=O)NC)nc3)c2=O)c2cccc(C)c21] |
| 123 | 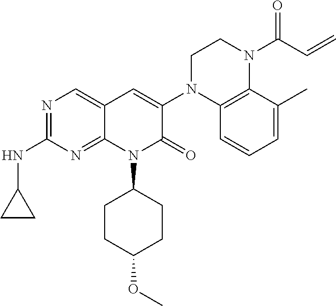 [SMILES: C=CC(=O)N1CCN(c2cc3cnc(NC4CC4)nc3n([C@H]3CC[C@H](OC)CC3)c2=O)c2cccc(C)c21] |
| 124 | 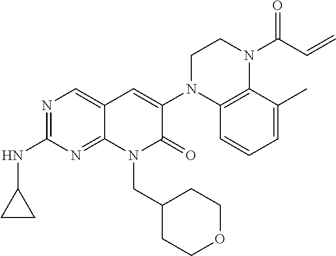 [SMILES: C=CC(=O)N1CCN(c2cc3cnc(NC4CC4)nc3n(CC3CCOCC3)c2=O)c2cccc(C)c21] |
| 125 | 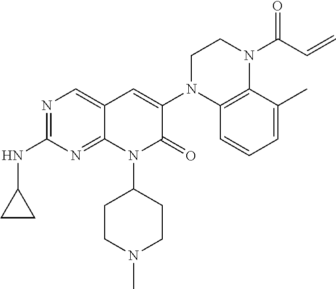 [SMILES: C=CC(=O)N1CCN(c2cc3cnc(NC4CC4)nc3n(C3CCN(C)CC3)c2=O)c2cccc(C)c21] |
| 126 | 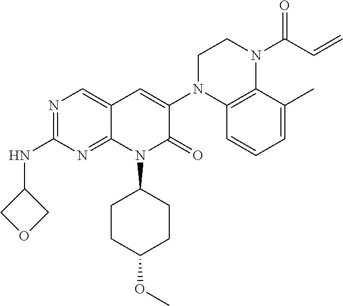 [SMILES: C=CC(=O)N1CCN(c2cc3cnc(NC4COC4)nc3n([C@H]3CC[C@H](OC)CC3)c2=O)c2cccc(C)c21] |
| 127 | 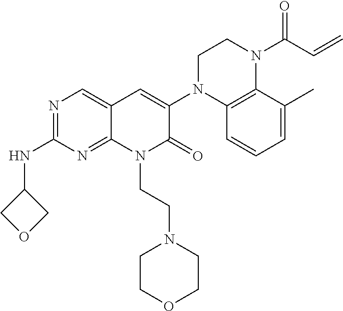 [SMILES: C=CC(=O)N1CCN(c2cc3cnc(NC4COC4)nc3n(CCN3CCOCC3)c2=O)c2cccc(C)c21] |
| 128 | 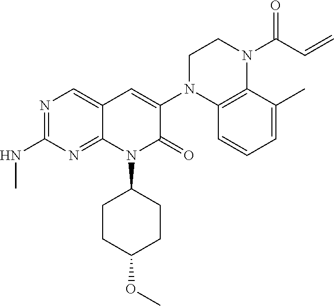 [SMILES: C=CC(=O)N1CCN(c2cc3cnc(NC)nc3n([C@H]3CC[C@H](OC)CC3)c2=O)c2cccc(C)c21] |
| 129 | 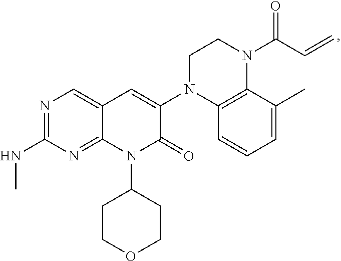 [SMILES: C=CC(=O)N1CCN(c2cc3cnc(NC)nc3n(C3CCOCC3)c2=O)c2cccc(C)c21] |

or a pharmaceutically acceptable salt thereof.

The compounds disclosed herein may exist as tautomers and optical isomers (e.g., enantiomers, diastereomers, diastereomeric mixtures, racemic mixtures, and the like).

It is generally well known in the art that any compound that will be converted in vivo to provide a compound disclosed herein is a prodrug within the scope of the present disclosure.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In an aspect, provided herein is a pharmaceutical composition comprising any one of the compounds disclosed herein, or a pharmaceutically acceptable salt thereof, and at least one pharmaceutically acceptable carrier.

In an embodiment, the composition further comprises a second active agent. In another embodiment, the second active agent is selected from the group consisting of a MEK inhibitor, a PI3K inhibitor, and an mTor inhibitor. In yet another embodiment, the second active agent prevents EGFR dimer formation in a subject. In still another embodiment, the second active agent is selected from the group consisting of cetuximab, trastuzumab, and panitumumab. In an embodiment, the second active agent is an ATP competitive EGFR inhibitor. In another embodiment, the ATP competitive EGFR inhibitor is osimertinib, gefitinib, or erlotinib. In another embodiment, the ATP competitive EGFR inhibitor is osimertinib.

In another aspect, provided herein are pharmaceutical compositions comprising a compound of the present disclosure, or a pharmaceutically acceptable salt thereof, and a pharmaceutically acceptable carrier.

In another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

In another aspect, the pharmaceutical composition further comprises a second active agent, wherein said second active agent prevents EGFR dimer formation, and a pharmaceutically acceptable carrier. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

A compound that binds to an allosteric site in EGFR, such as the compounds of the present disclosure (e.g., the compounds of the formulae disclosed herein), optionally in combination with a second active agent, wherein said second active agent prevents EGFR dimer formation, are capable of modulating EGFR activity. In some embodiments, the compounds of the present disclosure are capable of inhibiting or decreasing EGFR activity without a second active agent (e.g., an antibody such as cetuximab, trastuzumab, or panitumumab). In other embodiments, the compounds of the present disclosure in combination with a second active agent. In an embodiment, the second active agent prevents EGFR dimer formation and/or are capable of inhibiting or decreasing EGFR activity. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

### Methods of Treatment

In an aspect, provided herein is a method of treating cancer in an individual in need thereof, comprising administering to the individual a therapeutically effective amount of a compound disclosed herein, or a pharmaceutically acceptable salt thereof. In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another aspect, provided herein is a method of inhibiting the activity of EGFR in an subject in need thereof, comprising administering to the subject a therapeutically effective amount of a compound provided herein, or a pharmaceutically acceptable salt thereof.

In yet another aspect, provided herein is a method of inhibiting a kinase in an individual in need thereof, comprising administering to the individual a therapeutically effective amount of a compound provided herein. In an embodiment, the kinase is EGFR. In another embodiment, the kinase is HER.

In yet another aspect, provided herein is a method of treating or preventing a kinase-mediated disorder in an individual in need thereof, comprising administering to the individual a therapeutically effective amount of a compound of the present disclosure. In an embodiment, the kinase-mediated disorder is resistant to an EGFR-targeted therapy. In another embodiment, the EGFR-treated therapy is selected from the group consisting of gefitinib, erlotinib, osimertinib, CO-1686, and WZ4002.

In still another aspect, provided herein is a method of inhibiting the activity of EGFR in a subject in need thereof comprising targeting both Cys775 and Cys797 on EGFR. In yet another aspect, provided herein is a method of inhibiting the activity of EGFR in a subject in need thereof comprising administering a compound that targets both Cys775 and Cys797 on EGFR. The compound can simultaneously form two covalent bonds to cysteine 797 and cysteine 775. In an embodiment, the compound is a compound of Formula I, described herein.

In some embodiments, the compounds of the present disclosure are capable of modulating (e.g., inhibiting or decreasing) the activity of EGFR containing one or more mutations. In some embodiments, the mutant EGFR contains one or more mutations selected from T790M, L718Q, L844V, V948R, L858R, 1941R, C₇₉₇S, and Del. In other embodiments, the mutant EGFR contains a combination of mutations, wherein the combination is selected from Del/L718Q, Del/L844V, Del/T790M, Del/T790M/L718Q, Del/T790M/L844V, L858R/L718Q, L858R/L844V, L858R/T790M, L858R/T790M/1941R, Del/T790M, Del/T790M/C₇₉₇S, L858R/T790M/C₇₉₇S, and L858R/T790M/L718Q. In other embodiments, the mutant EGFR contains a combination of mutations, wherein the combination is selected from Del/L844V, L858R/L844V, L858R/T790M, L858R/T790M/1941R, L858R/T790M/C₇₉₇S, Del/T790M, Del/T790M, Del/T790M/C₇₉₇S, and L858R/T790M. In other embodiments, the mutant EGFR contains a combination of mutations, wherein the combination is selected from L858R/T790M, L858R/T790M/1941R, L858R/T790M/C₇₉₇S, Del/T790M, Del/T790M/C₇₉₇S, and L858R/T790M.

In some embodiments, the compounds of the present disclosure in combination with a second active agent, wherein said second active agent prevents EGFR dimer formation, are capable of modulating (e.g., inhibiting or decreasing) the activity of EGFR containing one or more mutations. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib.

In some embodiments, the compounds of the present disclosure are capable of modulating (e.g., inhibiting or decreasing) the activity of EGFR containing one or more mutations, but do not affect the activity of a wild-type EGFR.

Modulation of EGFR containing one or more mutations, such as those described herein, but not a wild-type EGFR, provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the compounds of the disclosure exhibit greater inhibition of EGFR containing one or more mutations as described herein relative to a wild-type EGFR. In certain embodiments, the compounds of the disclosure exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the compounds of the disclosure exhibit up to 1000-fold greater inhibition of EGFR containing one or more mutations as described herein relative to a wild-type EGFR. In various embodiments, the compounds of the disclosure exhibit up to 10000-fold greater inhibition of EGFR having a combination of mutations described herein (e.g., L858R/T790M, L858R/T790M/1941R, L858R/T790M/C₇₉₇S, Del/T790M, Del/T790M/C₇₉₇S, and L858R/T790M) relative to a wild-type EGFR.

In some embodiments, the inhibition of EGFR activity is measured by IC₅₀. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

In some embodiments, the inhibition of EGFR activity is measured by EC₅₀. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

In some embodiments, the inhibition of EGFR by a compound of the disclosure can be measured via a biochemical assay. By illustrative and non-limiting example, a homogenous time-resolved fluorescence (HTRF) assay may be used to determine inhibition of EGFR activity using conditions and experimental parameters disclosed herein. The HTRF assay may, for example, employ concentrations of substrate (e.g., biotin-Lck-peptide substrate) of about 1 μM; concentrations of EGFR (mutant or WT) from about 0.2 nM to about 40 nM; and concentrations of inhibitor from about 0.000282 μM to about 50 μM. A compound of the disclosure screened under these conditions may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM. In certain embodiments, a compound of the disclosure screened under the above conditions for inhibition of EGFR having a mutation or combination of mutations selected from L858R/T790M, L858R, and T790M may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

In some embodiments, the compounds of the disclosure bind to an allosteric site in EGFR. In some embodiments, the compounds of the disclosure interact with at least one amino acid residue of epidermal growth factor receptor (EGFR) selected from Lys745, Leu788, and Ala 743. In other embodiments, the compounds of the disclosure interact with at least one amino acid residue of epidermal growth factor receptor (EGFR) selected from Cys755, Leu777, Phe856, and Asp855. In other embodiments, the compounds of the disclosure interact with at least one amino acid residue of epidermal growth factor receptor (EGFR) selected from Met766, Ile759, Glu762, and Ala763. In other embodiments, the compounds of the disclosure interact with at least one amino acid residue of epidermal growth factor receptor (EGFR) selected from Lys745, Leu788, and Ala 743; at least one amino acid residue of epidermal growth factor receptor (EGFR) selected from Cys755, Leu777, Phe856, and Asp855; and at least one amino acid residue of epidermal growth factor receptor (EGFR) selected from Met766, Ile759, Glu762, and Ala763. In other embodiments, the compounds of the disclosure do not interact with any of the amino acid residues of epidermal growth factor receptor (EGFR) selected from Met793, Gly796, and Cys797.

An EGFR sensitizing mutation comprises without limitation L858R, G719S, G719C, G719A, L861Q, a deletion in exon 19 and/or an insertion in exon 20. A drug-resistant EGFR mutant can have without limitation a drug resistance mutation comprising T790M, T854A, L718Q, C₇₉₇S, or D761Y.

The selectivity between wild-type EGFR and EGFR containing one or more mutations as described herein can also be measured using cellular proliferation assays where cell proliferation is dependent on kinase activity. For example, murine Ba/F3 cells transfected with a suitable version of wild-type EGFR (such as VIII; containing a WT EGFR kinase domain), or Ba/F3 cells transfected with L858R/T790M, Del/T790M/L718Q, L858R/T790M/L718Q, L858R/T790M/C₇₉₇S, Del/T790M/C₇₉₇S, L858R/T790M/1941R, or Exon 19 deletion/T790M can be used. Proliferation assays are performed at a range of inhibitor concentrations (10 μM, 3 μM, 1.1 μM, 330 nM, 110 nM, 33 nM, 11 nM, 3 nM, 1 nM) and an EC₅₀ is calculated.

In still another aspect, the disclosure provides a method of inhibiting epidermal growth factor receptor (EGFR), the method comprising administering to a subject in need thereof an effective amount of a compound disclosed herein, or a pharmaceutically acceptable salt thereof. In some embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In another aspect, provided herein is a method of treating or preventing a disease, the method comprising administering to a subject in need thereof an effective amount of a compound disclosed herein, or a pharmaceutically acceptable salt thereof. In some embodiments, the disease is mediated by a kinase. In further embodiments, the kinase comprises a mutated cysteine residue. In further embodiments, the mutated cysteine residue is located in or near the position equivalent to Cys 797 in EGFR, including such positions in Jak3, Blk, Bmx, Btk, HER2 (ErbB2), HER4 (ErbB4), Itk, Tec, and Txk. In some embodiments, the method further comprises administering a second active agent, wherein said second active agent prevents dimer formation of the kinase. In some embodiments, the second active agent that prevents kinase dimer formation is an antibody. In further embodiments, the second active agent prevents EGFR dimer formation. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disease is mediated by EGFR (e.g., EGFR plays a role in the initiation or development of the disease). In some embodiments, the disease is mediated by a Her-kinase. In further embodiments, the Her-kinase is HER1, HER2, or HER4.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C₇₉₇S, or D761Y. The diagnostic test can comprise sequencing, pyrosequencing, PCR, RT-PCR, or similar analysis techniques known to those of skill in the art that can detect nucleotide sequences.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C₇₉₇S, or D761Y. The diagnostic test can comprise sequencing, pyrosequencing, PCR, RT-PCR, or similar analysis techniques known to those of skill in the art that can detect nucleotide sequences.

In yet another aspect, provided herein is a method of treating a kinase-mediated disorder comprising administering to a subject in need thereof an effective amount of a compound disclosed herein, or a pharmaceutically acceptable salt thereof. In some embodiments, the compound is an inhibitor of HER1, HER2, or HER4. In other embodiments, the subject is administered an additional therapeutic agent. In other embodiments, the compound and the additional therapeutic agent are administered simultaneously or sequentially.

In another aspect, the disclosure provides a method of treating a kinase mediated disorder, the method comprising administering to a subject in need thereof an effective amount of a compound disclosed herein, or a pharmaceutically acceptable salt thereof, and a second active agent, wherein said second active agent prevents EGFR dimer formation. In some embodiments, the compound is an inhibitor of HER1, HER2, or HER4. In other embodiments, the subject is administered an additional therapeutic agent. In other embodiments, the compound, the second active agent that prevents EGFR dimer formation, and the additional therapeutic agent are administered simultaneously or sequentially. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In other embodiments, the disease is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the EGFR activation is selected from mutation of EGFR, amplification of EGFR, expression of EGFR, and ligand mediated activation of EGFR.

In further embodiments, the mutation of EGFR is selected from G719S, G719C, G719A, L858R, L861Q, an exon 19 deletion mutation, and an exon 20 insertion mutation.

In still another aspect, provided herein is a method of treating cancer in a subject, wherein the subject is identified as being in need of EGFR inhibition for the treatment of cancer, comprising administering to the subject an effective amount of a compound disclosed herein, or a pharmaceutically acceptable salt thereof.

In certain embodiments, the subject identified as being in need of EGFR inhibition is resistant to a known EGFR inhibitor, including but not limited to, gefitinib, erlotinib, osimertinib, CO-1686, or WZ4002. In certain embodiments, a diagnostic test is performed to determine if the subject has an activating mutation in EGFR. In certain embodiments, a diagnostic test is performed to determine if the subject has an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C₇₉₇S, or D761Y. The diagnostic test can comprise sequencing, pyrosequencing, PCR, RT-PCR, or similar analysis techniques known to those of skill in the art that can detect nucleotide sequences.

In an aspect, provided herein is a method of preventing resistance to a known EGFR inhibitor (including but not limited to gefitinib, erlotinib, osimertinib, CO-1686, or WZ4002) in a subject, comprising administering to a subject in need thereof an effective amount of a compound disclosed herein, or a pharmaceutically acceptable salt thereof.

In another aspect, provided herein is a method of preventing resistance to a known EGFR inhibitor (including but not limited to gefitinib, erlotinib, osimertinib, CO-1686, or WZ4002) in a disease, comprising administering to a subject in need thereof an effective amount of a compound disclosed herein, or a pharmaceutically acceptable salt thereof, and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which EGFR plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis,

Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof. In other embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The activity of the compounds and compositions of the present disclosure as EGFR kinase inhibitors may be assayed in vitro, in vivo, or in a cell line. In vitro assays include assays that determine inhibition of either the kinase activity or ATPase activity of the activated kinase.

Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this disclosure as an inhibitor of various kinases are set forth in the Examples below.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent, wherein said second active agent prevents EGFR dimer formation. For any of the above uses, the required dosage will vary depending on the mode of administration, the particular condition to be treated and the effect desired.

In other embodiments, the compound and the second active agent that prevents EGFR dimer formation are administered simultaneously or sequentially.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, an agent that prevents EGFR dimer formation, chemotherapeutic agents or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer. In certain embodiments, the kit further comprises components for performing a test to determine whether a subject has activating and/or drug resistance mutations in EGFR.

In another aspect, the disclosure provides a kit comprising a compound capable of inhibiting EGFR activity selected from a compound disclosed herein, or a pharmaceutically acceptable salt thereof.

In another aspect, the disclosure provides a kit comprising a compound capable of inhibiting kinase activity selected from one or more compounds of disclosed herein, or pharmaceutically acceptable salts thereof; a second active agent, wherein said second active agent prevents EGFR dimer formation; and instructions for use in treating cancer. In certain embodiments, the kit further comprises components for performing a test to determine whether a subject has activating and/or drug resistance mutations in EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In another aspect, the disclosure provides a kit comprising a compound capable of inhibiting EGFR activity selected from a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Examples

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Abbreviations

ACN acetonitrile
AcOH acetic acid
BPD bis(pinacolato)diboron
DIAD diisopropyl azodicarboxylate
DCE dichloroethane
DCM dichloromethane
XPhos Pd G3 (2-dicyclohexylphosphino-2′,4′,6′-triisopropyl-1,1′-biphenyl) [2-(2′-amino-1,1′-biphenyl)]palladium (II) methanesulfonate
DIEA/DIPEA diisopropylethylamine
DMAP 4-dimethylaminopyridine
DMF dimethylformamide
Xantphos (9,9-dimethyl-9H-xanthene-4,5-diyl)bis(diphenylphosphane)
DPPA diphenylphosphoryl azide
EtOAc ethyl acetate
EtOH ethanol
eq equivalent(s)
g gram(s)
hr hour(s)
LC-MS liquid chromatography-mass spectrometry
LAH lithium aluminum hydride
m-CPBA meta-chloroperoxybenzoic acid
min minute(s)
MS mass spectrometry
MeOH methanol
μL microliter(s)
μm micrometer(s)
μmol micromole(s)
mg milligram(s)
mm millimeter(s)
mL milliliter(s)
mmol millimole(s)
psi pounds per square inch
Py pyridine
Selectfluor 1-(chloromethyl)-4-fluoro-1,4-diazabicyclo[2.2.2]octane-1,4-diium
ditetrafluoroborate
NaHDMS sodium bis(trimethylsilyl)amide
TEA triethylamine
TFA trifluoroacetic acid
Pd₂(dba)₃ tris(dibenzylideneacetone) dipalladium (0)
THF tetrahydrofuran
TLC thin layer chromatography

### Example 1: Synthetic Procedures

### Procedure for Preparation of Compound 001

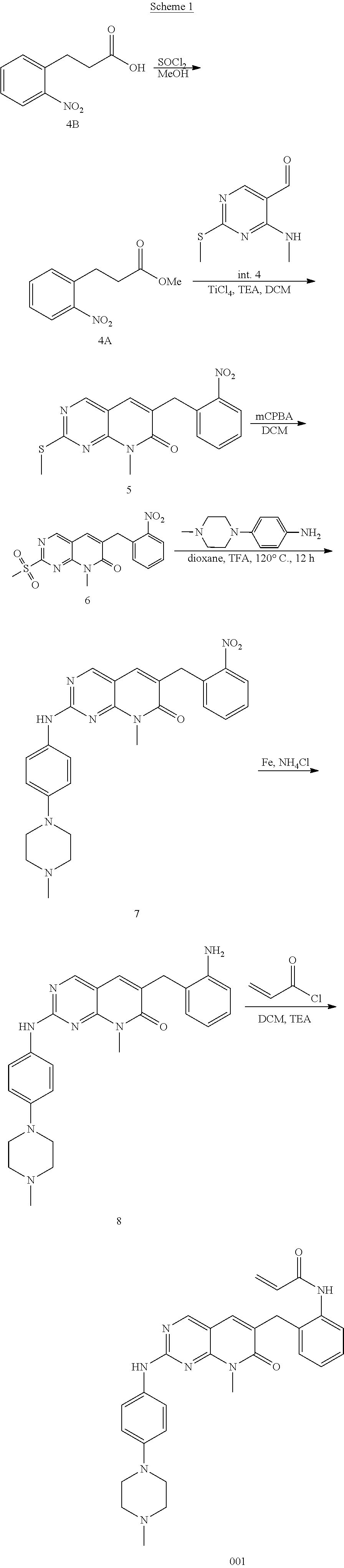

To a solution of 3-(2-nitrophenyl) propanoic acid (2 g, 10.25 mmol, 1 eq) in MeOH (30 mL) was added SOCl₂ (1.34 g, 11.27 mmol, 817.71 μL, 1.1 eq) at 0° C. The mixture was stirred at 25° C. for 12 hr. LC-MS showed the desired compound was detected. The reaction mixture was concentrated under reduced pressure to give methyl 3-(2-nitrophenyl) propanoate (2 g, crude) as a light yellow oil.

To a solution of 4-(methylamino)-2-methylsulfanyl-pyrimidine-5-carbaldehyde (1.3 g, 7.09 mmol, 1 eq) in DCM (40 mL) was added methyl 3-(2-nitrophenyl) propanoate (1.48 g, 7.09 mmol, 1 eq) and TiCl₄ (1.75 g, 9.22 mmol, 1.3 eq) was stirred at 25° C. for 30 min. Then TEA (2.87 g, 28.38 mmol, 3.95 mL, 4 eq) was added to the mixture. The mixture was stirred at 25° C. for 12 hr. LC-MS showed the desired compound was detected. The reaction mixture was partitioned between H₂O (50 mL) and dichloromethane (50 mL× 2). The organic phase was separated, washed with brine (30 mL× 2), dried over Na₂SO₄, filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @ 100 mL/min) to give 8-methyl-2-methylsulfanyl-6-[(2-nitrophenyl)methyl]pyrido[2,3-d]pyrimidin-7-one (850 mg, 2.48 mmol, 34.99% yield) as a yellow solid.

To a solution of 8-methyl-2-methylsulfanyl-6-[(2-nitrophenyl)methyl]pyrido[2,3-d]pyrimidin-7-one (800 mg, 2.34 mmol, 1 eq) in DCM (10 mL) was added m-CPBA (901.33 mg, 4.44 mmol, 85% purity, 1.9 eq) and DCM (10 mL) at 0° C. The mixture was stirred at 25° C. for 4 hr. LC-MS showed the desired compound was detected. The residue was diluted with Na₂SO₃ (50 mL) and extracted with dichloromethane (50 mL× 3). The combined organic layers were washed with NaHCO₃ (50 mL×3), dried over Na₂SO₄, filtered and concentrated under reduced pressure to give 8-methyl-2-methylsulfonyl-6-[(2-nitrophenyl)methyl]pyrido[2,3-d]pyrimidin-7-one (800 mg, crude) as a yellow solid.

To a solution of 8-methyl-2-methylsulfonyl-6-[(2-nitrophenyl)methyl]pyrido[2,3-d]pyrimidin-7-one (300 mg, 801.34 μmol, 1 eq) in dioxane (4 mL) was added TFA (137.06 mg, 1.20 mmol, 89.00 μL, 1.5 eq) and 4-(4-methylpiperazin-1-yl)aniline (229.91 mg, 1.20 mmol, 1.5 eq). The mixture was stirred at 120° C. for 12 hr. LC-MS showed the desired compound was detected. The reaction mixture was partitioned between NaHCO₃ (30 mL) and ethyl acetate (30 mL× 3). The organic phase was separated, washed with brine (20 mL× 2), dried over Na₂SO₄, filtered and concentrated under reduced pressure to give a residue. The residue was purified by flash silica gel chromatography (ISCO®; 12 g SepaFlash® Silica Flash Column, Eluent of 0˜20% Dichloromethane/Methanol@ 80 mL/min) to give 8-methyl-2-[4-(4-methylpiperazin-1-yl)anilino]-6-[(2-nitrophenyl)methyl]pyrido[2,3-d]pyrimidin-7-one (250 mg, 514.89 μmol, 64.25% yield) as a yellow solid.

To a solution of 8-methyl-2-[4-(4-methylpiperazin-1-yl)anilino]-6-[(2-nitrophenyl)methyl]pyrido[2,3-d]pyrimidin-7-one (140 mg, 288.34 μmol, 1 eq) in EtOH (6 mL) and H₂O (2 mL) was added NH₄CI (30.85 mg, 576.68 μmol, 2 eq) and stirred at 25° C. for 5 min. Then the mixture was heated to 90° C., Fe (64.41 mg, 1.15 mmol, 4 eq) was added to the mixture. The mixture was stirred at 90° C. for 1 hr. LC-MS showed the desired compound was detected. The reaction mixture was filtered and the filtrate dried in vacuum to a residue. The residue was washed with the solvent 80 mL (dichloromethane: Methanol=10:1) and filtered and the filtrate dried in vacuum to give 6-[(2-aminophenyl)methyl]-8-methyl-2-[4-(4-methylpiperazin-1-yl)anilino]pyrido[2,3-d]pyrimidin-7-one (120 mg, crude) as a yellow solid.

To a solution of 6-[(2-aminophenyl)methyl]-8-methyl-2-[4-(4-methylpiperazin-1-yl)anilino]pyrido[2,3-d]pyrimidin-7-one (100 mg, 219.51 μmol, 1 eq) and TEA (66.64 mg, 658.54 μmol, 91.66 μL, 3 eq) in DCM (2 mL) was added prop-2-enoyl chloride (19.87 mg, 219.51 μmol, 17.90 μL, 1 eq) in DCM (0.2 mL) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 100*30 mm*5 μm; mobile phase: [water (FA)-ACN];B %: 5%-45%, 10 min) to give N-[2-[[8-methyl-2-[4-(4-methylpiperazin-1-yl)anilino]-7-oxo-pyrido[2,3-d]pyrimidin-6-yl]methyl]phenyl]prop-2-enamide (10.5 mg, 17.63 μmol, 8.03% yield, 93.28% purity, FA) as a yellow solid.

### Procedure for Preparation of Compound 002

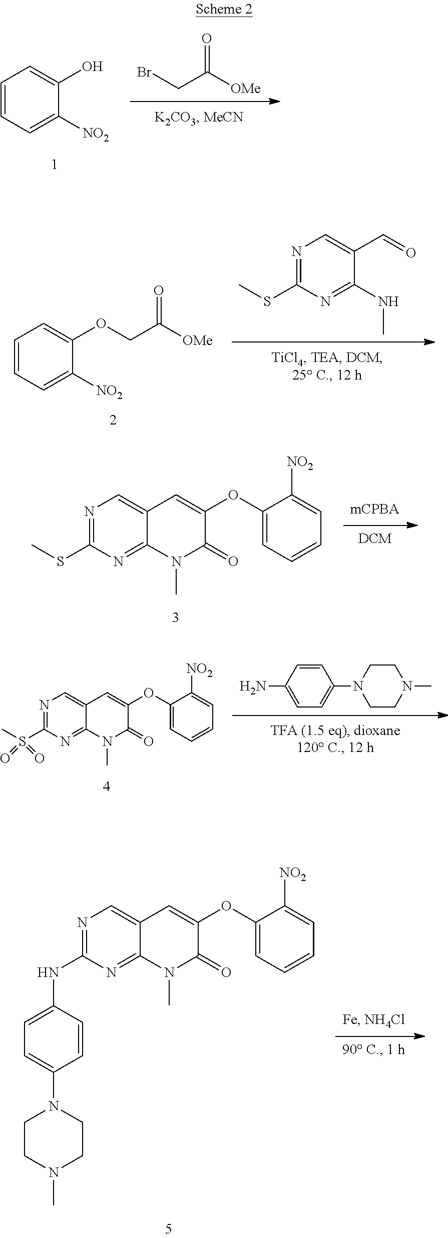

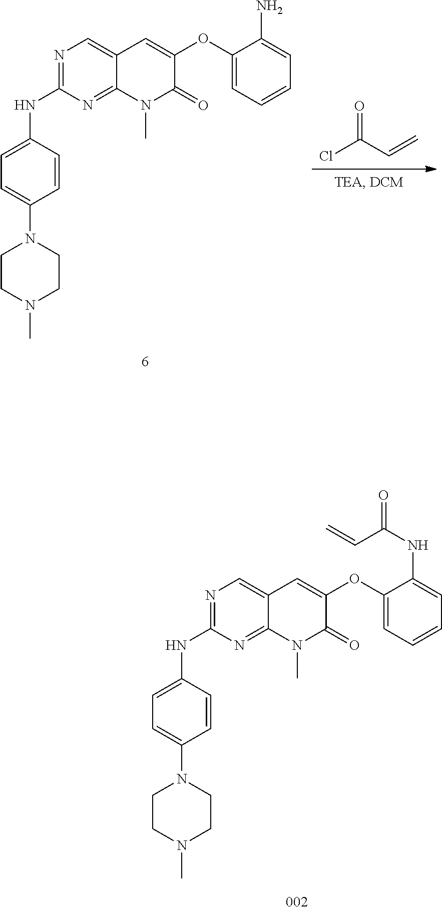

To a solution of 2-nitrophenol (5 g, 35.94 mmol, 1 eq) in MeCN (100 mL) was added K₂CO₃ (9.94 g, 71.89 mmol, 2 eq) and methyl 2-bromoacetate (6.60 g, 43.13 mmol, 4.07 mL, 1.2 eq). The mixture was stirred at 25° C. for 12 hr and 60° C. for 3 hr. LC-MS showed the desired compound was detected. The reaction mixture was partitioned between water (100 mL) and ethyl acetate (100 mL× 2). The organic phase was separated, washed with brine (50 ml×1), dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 100 mL/min) to give methyl 2-(2-nitrophenoxy)acetate (7 g, 33.15 mmol, 92.22% yield) as a light yellow solid.

To a solution of methyl 2-(2-nitrophenoxy)acetate (1.5 g, 7.10 mmol, 1 eq) in DCM (40 mL) was added 4-(methylamino)-2-methylsulfanyl-pyrimidine-5-carbaldehyde (1.30 g, 7.10 mmol, 1 eq) and TiCl₄ (1.75 g, 9.23 mmol, 1.3 eq) stirred at 25° C. for 5 min. Then TEA (2.88 g, 28.41 mmol, 3.95 mL, 4 eq) was added to the mixture. The mixture was stirred at 25° C. for 12 hr. LC-MS showed the desired compound was detected. The reaction mixture was partitioned between H₂O (50 mL) and dichloromethane (50 mL× 2). The organic phase was separated, washed with brine (30 mL× 2), dried over Na₂SO₄, filtered and concentrated under reduced pressure to give a residue. The residue was purified by flash silica gel chromatography (ISCO®; 20 g SepaFlash® Silica Flash Column, Eluent of 0˜20% Ethyl acetate/Petroleum ether gradient @ 100 mL/min) to give 8-methyl-2-methylsulfanyl-6-(2-nitrophenoxy)pyrido[2, 3-d]pyrimidin-7-one (900 mg, 2.61 mmol, 36.80% yield) as a yellow solid.

A solution of m-CPBA (1.41 g, 6.97 mmol, 85% purity, 4 eq) in DCM (10 mL) was added to 8-methyl-2-methylsulfanyl-6-(2-nitrophenoxy)pyrido[2,3-d]pyrimidin-7-one (600 mg, 1.74 mmol, 1 eq) in DCM (10 mL) at 0° C. The mixture was stirred at 25° C. for 12 hr. LC-MS showed the desired compound was detected. The residue was diluted with Na₂SO₃ (50 mL) and extracted with dichloromethane (50 mL× 3). The combined organic layers were washed with NaHCO₃ (30 mL× 3), dried over Na₂SO₄, filtered and concentrated under reduced pressure to give 8-methyl-2-methylsulfonyl-6-(2-nitrophenoxy)pyrido[2,3-d]pyrimidin-7-one (600 mg, crude) as a light yellow solid.

To a solution of 8-methyl-2-methylsulfonyl-6-(2-nitrophenoxy)pyrido[2,3-d]pyrimidin-7-one (600 mg, 1.59 mmol, 1 eq) in dioxane (10 mL) was added TFA (272.68 mg, 2.39 mmol, 177.06 μL, 1.5 eq) and 4-(4-methylpiperazin-1-yl)aniline (457.41 mg, 2.39 mmol, 1.5 eq). The mixture was stirred at 120° C. for 12 hr. LC-MS showed the desired compound was detected. The reaction mixture was partitioned between NaHCO₃ (50 mL) and ethyl acetate (50 mL×3). The organic phase was separated, washed with brine (30 mL× 2), dried over Na₂SO₄, filtered and concentrated under reduced pressure to give a residue. The residue was purified by flash silica gel chromatography (ISCO®; 12 g SepaFlash® Silica Flash Column, Eluent of 0˜20% Dichloromethane/Methanol@ 80 mL/min) to give 8-methyl-2-[4-(4-methylpiperazin-1-yl) anilino]-6-(2-nitrophenoxy)pyrido[2,3-d]pyrimidin-7-one (250 mg, 512.81 μmol, 32.17% yield) as a yellow solid.

To a solution of 8-methyl-2-[4-(4-methylpiperazin-1-yl)anilino]-6-(2-nitrophenoxy)pyrido[2,3-d]pyrimidin-7-one (250 mg, 512.81 μmol, 1 eq) in EtOH (9 mL) and H₂O (3 mL) was added NH₄CI (54.86 mg, 1.03 mmol, 2 eq) and stirred at 25° C. for 5 min. Then the mixture was heated to 90° C., Fe (114.55 mg, 2.05 mmol, 4 eq) was added to the mixture. The mixture was stirred at 90° C. for 1 hr. LC-MS showed the desired compound was detected. The reaction mixture was filtered and the filtrate dried in vacuum to a residue. The residue was washed with the solvent 50 mL (dichloromethane: Methanol=10:1) and filtered and the filtrate dried in vacuum to give 6-(2-aminophenoxy)-8-methyl-2-[4-(4-methylpiperazin-1-yl)anilino]pyrido[2,3-d]pyrimidin-7-one (140 mg, crude) was obtained as a light yellow solid.

To a solution of 6-(2-aminophenoxy)-8-methyl-2-[4-(4-methylpiperazin-1-yl)anilino]pyrido[2,3-d]pyrimidin-7-one (70 mg, 153.00 μmol, 1 eq) and TEA (46.44 mg, 458.99 μmol, 63.89 μL, 3 eq) in DCM (2 mL) was added a DCM solution of prop-2-enoyl chloride (11.08 mg, 122.40 μmol, 9.98 μL, 0.8 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 100×30 mm×5 μm; mobile phase: [water (FA)-ACN];B %: 5%-45%, 10 min) and (NH₄HCO₃ condition, column: Waters Xbridge BEH C18 100×30 mm×10 μm; mobile phase: [water (NH₄HCO₃)-ACN];B %: 20%-50%, 8 min) to give N-[2-[8-methyl-2-[4-(4-methylpiperazin-1-yl)anilino]-7-oxo-pyrido[2,3-d]pyrimidin-6-yl]oxyphenyl]prop-2-enamide (4.19 mg, 8.19 μmol, 5.35% yield, 100% purity) as a yellow solid.

### Procedure for Preparation of Compounds 003, 004, and 005

Compounds 003, 004, and 005 were prepared as described for the synthesis of compound 002.

### Procedure for Preparation of Compound 006

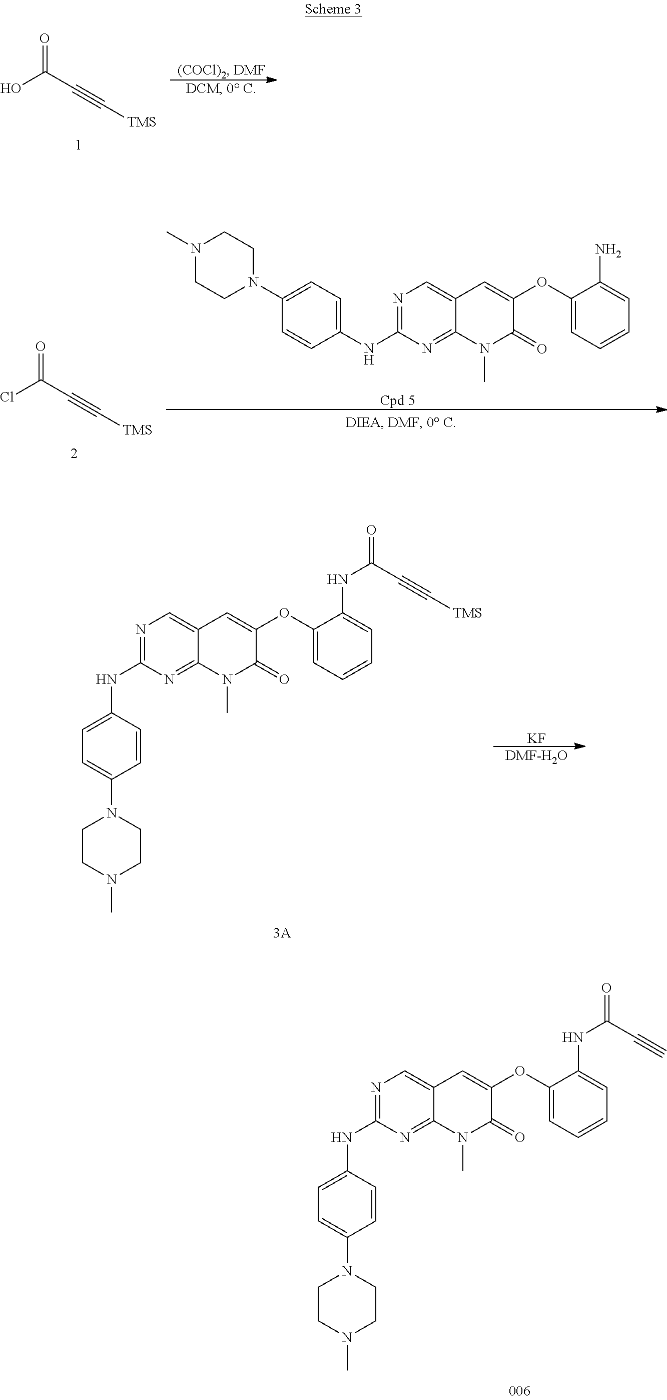

To a solution of 3-trimethylsilylprop-2-ynoic acid (100 mg, 703.1 μmol, 1 eq) in DCM (2 mL) was added DMF (513.9 ug, 7.0 μmol, 5.41^{e-1} μL, 0.01 eq) and oxalyl dichloride (133.9 mg, 1.0 mmol, 92.3 μL, 1.5 eq) at 0° C. The reaction mixture was stirred at 25° C. for 1 hr. TLC plate1 indicated Reactant 1 was consumed completely and one new spot formed. The reaction mixture was concentrated under reduced pressure to give crude product 3-trimethylsilylprop-2-ynoyl chloride (120 mg, crude) as yellow oil.

To a solution of 6-(2-aminophenoxy)-8-methyl-2-[4-(4-methylpiperazin-1-yl)anilino]pyrido[2,3-d]pyrimidin-7-one (230 mg, 502.70 μmol, 1 eq) in DMF (2 mL) was added DIEA (64.9 mg, 502.7 μmol, 87.6 μL, 1 eq) and 3-trimethylsilylprop-2-ynoyl chloride (80.8 mg, 502.7 μmol, 1 eq). The mixture was stirred at 0° C. for 10 min. The reaction was monitored by LCMS which showed the reactant was consumed completely and the desired mass peak was detected. The reaction mixture was filtered. The residue was purified by prep-HPLC (FA condition; column: Phenomenex Luna C18 75*30 mm*3 μm; mobile phase: [water (FA)-ACN];B %: 20%-50%, 8 min) to yield N-[2-[8-methyl-2-[4-(4-methylpiperazin-1-yl)anilino]-7-oxo-pyrido[2,3-d]pyrimidin-6-yl]oxyphenyl]-3-trimethylsilyl-prop-2-ynamide (100 mg, 171.9 μmol, 34.2% yield) obtained as yellow solid.

To a solution of N-[2-[8-methyl-2-[4-(4-methylpiperazin-1-yl)anilino]-7-oxo-pyrido[2,3-d]pyrimidin-6-yl]oxyphenyl]-3-trimethylsilyl-prop-2-ynamide (10 mg, 17.2 μmol, 1 eq) in DMF (1 mL)/H₂O (0.4 mL) was added KF (2.5 mg, 42.9 μmol, 1.0 μL, 2.5 eq). The mixture was stirred at 25° C. for 3 hr. The reaction was monitored by LCMS which showed complete consumption of reactant and detection of the desired mass peak. The residue was purified by prep-HPLC (FA condition; column: Phenomenex Luna C18 75*30 mm*3 μm; mobile phase: [water (FA)-ACN];B %: 1%-40%, 8 min) to yield N-[2-[8-methyl-2-[4-(4-methylpiperazin-1-yl)anilino]-7-oxo-pyrido[2,3-d]pyrimidin-6-yl]oxyphenyl]prop-2-ynamide (6 mg, 8.8 μmol, 51.2% yield, 81.561% purity, FA) obtained as a yellow solid. Analytic data QC were consistent with the desired product.

### Procedure for Preparation of Compound 007

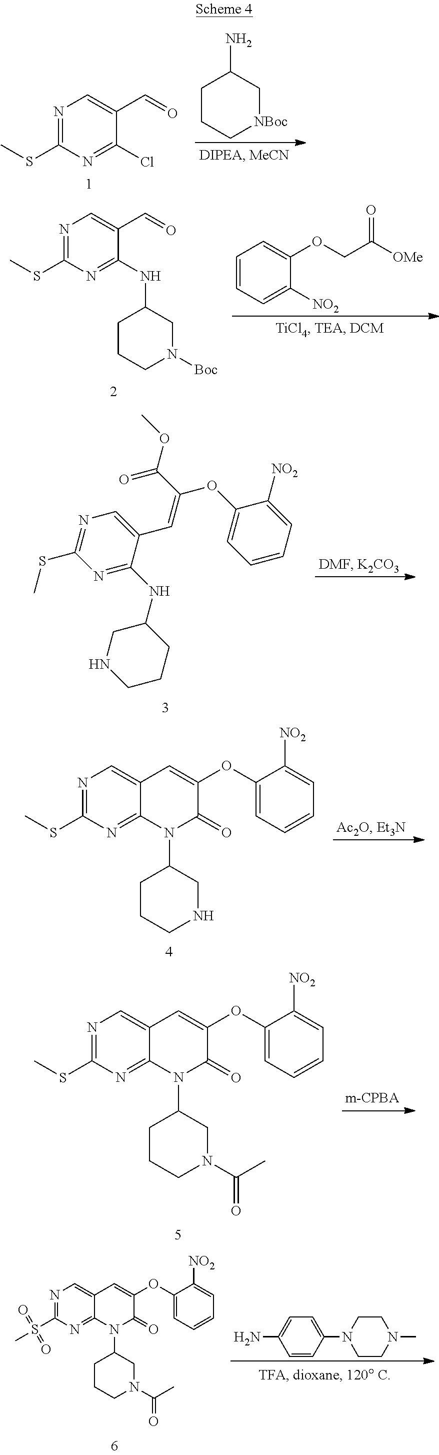

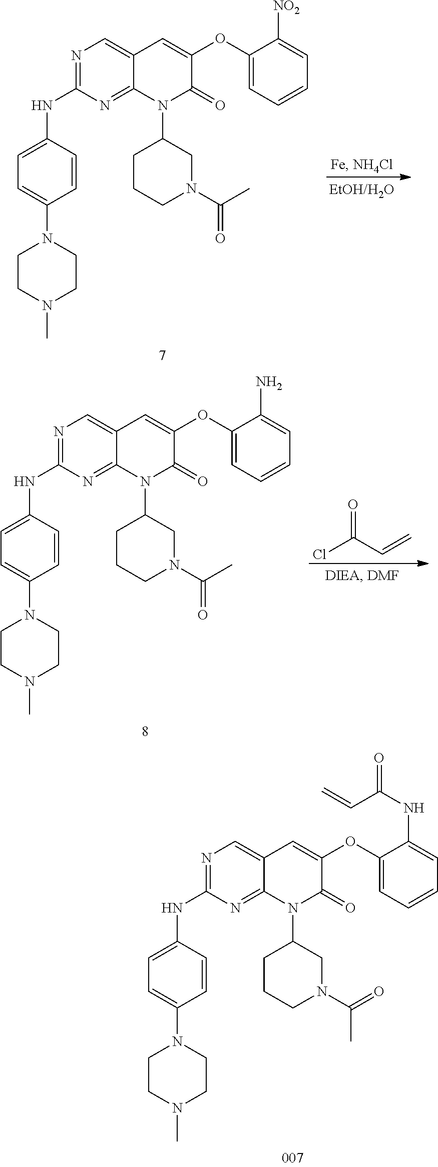

To a solution of 4-chloro-2-methylsulfanyl-pyrimidine-5-carbaldehyde (2 g, 10.60 mmol, 1 eq) in MeCN (20 mL) was added DIEA (4.11 g, 31.81 mmol, 5.54 mL, 3 eq) and tert-butyl 3-aminopiperidine-1-carboxylate (2.34 g, 11.66 mmol, 1.1 eq). The mixture was stirred at 40° C. for 12 hr. LC-MS showed the desired compound was detected. The reaction mixture was concentrated under reduced pressure. The residue was purified by flash silica gel chromatography (ISCO®; 25 g SepaFlash® Silica Flash Column, Eluent of 0˜50% Ethyl acetate/Petroleum ether gradient @ 100 mL/min) to give tert-butyl 3-[(5-formyl-2-methylsulfanyl-pyrimidin-4-yl)amino]piperidine-1-carboxylate (2 g, 5.67 mmol, 53.52% yield) as a light yellow solid. 10

To a solution of tert-butyl 3-[(5-formyl-2-methylsulfanyl-pyrimidin-4-yl)amino]piperidine-1-carboxylate (2 g, 5.67 mmol, 1 eq) in DCM (100 mL) was added methyl 2-(2-nitrophenoxy)acetate (1.08 g, 5.11 mmol, 0.9 eq) and TiCl₄ (1.61 g, 8.51 mmol, 1.5 eq) was stirred at 25° C. for 30 min. Then TEA (2.58 g, 25.54 mmol, 3.55 mL, 4.5 eq) was added to the mixture. The mixture was stirred at 25° C. for 12 hr. LC-MS showed the desired compound was detected. The reaction mixture was partitioned between water (100 mL) and dichloromethane (100 mL× 2). The organic phase was separated, washed with brine (50 mL× 1), dried over Na₂SO₄, filtered and concentrated under reduced pressure to give a residue. The residue was purified by flash silica gel chromatography (ISCO®; 40 g SepaFlash® Silica Flash Column, Eluent of 0˜20% Ethyl acetate/Petroleum ether gradient @ 120 mL/min) to give methyl (E)-3-[2-methylsulfanyl-4-(3-piperidylamino)pyrimidin-5-yl]-2-(2-nitrophenoxy) prop-2-enoate (700 mg, 1.28 mmol, 22.51% yield, 81.3% purity) as a white solid.

To a solution of methyl (E)-3-[2-methylsulfanyl-4-(3-piperidylamino)pyrimidin-5-yl]-2-(2-nitrophenoxy) prop-2-enoate (700 mg, 1.57 mmol, 1 eq) in DMF (15 mL) was added K₂CO₃ (651.49 mg, 4.71 mmol, 3 eq). The mixture was stirred at 80° C. for 12 hr. LC-MS showed the desired compound was detected. The crude product 2-methylsulfanyl-6-(2-nitrophenoxy)-8-(3-piperidyl)pyrido[2,3-d]pyrimidin-7-one (700 mg, crude) in DMF as a black liquid was used into the next step without further work up.

To a solution of 2-methylsulfanyl-6-(2-nitrophenoxy)-8-(3-piperidyl)pyrido[2,3-d]pyrimidin-7-one (650 mg, 1.57 mmol, 1 eq) in DMF (10 mL) was added DIEA (609.56 mg, 4.72 mmol, 821.51 μL, 3 eq) and Ac₂O (240.75 mg, 2.36 mmol, 220.87 μL, 1.5 eq). The mixture was stirred at 25° C. for 1 hr. LC-MS showed the desired compound was detected. The reaction mixture was partitioned between water (40 mL) and ethyl acetate (50 mL× 3). The organic phase was separated, washed with brine (20 mL× 2), dried over Na₂SO₄, filtered and concentrated under reduced pressure to give a residue. The residue was purified by flash silica gel chromatography (ISCO®; 20g SepaFlash® Silica Flash Column, Eluent of 0˜100% Ethyl acetate/Petroleum ether gradient @100 mL/min) to give 8-(1-acetyl-3-piperidyl)-2-methylsulfanyl-6-(2-nitrophenoxy)pyrido[2,3-d]pyrimidin-7-one (200 mg, 362.26 μmol, 23.04% yield, 90.29% purity) as a yellow solid.

To a solution of 8-(1-acetyl-3-piperidyl)-2-methylsulfanyl-6-(2-nitrophenoxy)pyrido[2,3-d]pyrimidin-7-one (170 mg, 373.23 μmol, 1 eq) in DCM (3 mL) was added m-CPBA (113.66 mg, 559.84 μmol, 85% purity, 1.5 eq) at 0° C. The mixture was stirred at 25° C. for 2 hr. LC-MS showed the desired compound was detected. The residue was diluted with Na₂SO₃ (30 mL) and extracted with dichloromethane (30 mL×3). The combined organic layers were washed with NaHCO₃ (30 mL× 3), dried over Na₂SO₄, filtered and concentrated under reduced pressure to give 8-(1-acetyl-3-piperidyl)-2-methylsulfonyl-6-(2-nitrophenoxy)pyrido[2,3-d]pyrimidin-7-one (180 mg, crude) as a light yellow oil.

To a solution of 8-(1-acetyl-3-piperidyl)-2-methylsulfonyl-6-(2-nitrophenoxy)pyrido[2,3-d]pyrimidin-7-one (180 mg, 369.24 μmol, 1 eq) in dioxane (2 mL) was added 4-(4-methylpiperazin-1-yl)aniline (84.75 mg, 443.09 μmol, 1.2 eq) and TFA (63.15 mg, 553.86 μmol, 41.01 μL, 1.5 eq). The mixture was stirred at 120° C. for 12 hr. LC-MS showed the desired compound was detected. The reaction mixture was concentrated under reduced pressure to give a residue. The residue was purified by prep-TLC (SiO₂, Dichloromethane: Methanol=8:1) to give 8-(1-acetyl-3-piperidyl)-2-[4-(4-methylpiperazin-1-yl)anilino]-6-(2-nitrophenoxy)pyrido[2,3-d]pyrimidin-7-one (130 mg, 217.15 μmol, 58.81% yield) as a yellow oil.

To a solution of 8-(1-acetyl-3-piperidyl)-2-[4-(4-methylpiperazin-1-yl)anilino]-6-(2-nitrophenoxy)pyrido[2,3-d]pyrimidin-7-one (130 mg, 217.15 μmol, 1 eq) in EtOH (6 mL) and H₂O (2 mL) was added NH₄CI (23.23 mg, 434.31 μmol, 2 eq) and Fe (48.51 mg, 868.62 μmol, 4 eq). The mixture was stirred at 80° C. for 1 hr. LC-MS showed the desired compound was detected. The reaction mixture was filtered and the filtrate dried in vacuum to give 8-(1-acetyl-3-piperidyl)-6-(2-aminophenoxy)-2-[4-(4-methylpiperazin-1-yl)anilino]pyrido[2,3-d]pyrimidin-7-one (100 mg, crude) as a yellow solid.

To a solution of 8-(1-acetyl-3-piperidyl)-6-(2-aminophenoxy)-2-[4-(4-methylpiperazin-1-yl)anilino]pyrido[2,3-d]pyrimidin-7-one (80 mg, 140.68 μmol, 1 eq) and DIEA (54.54 mg, 422.04 μmol, 73.51 μL, 3 eq) in DMF (2 mL) was added a DCM solution of prop-2-enoyl chloride (25.47 mg, 281.36 μmol, 22.94 μL, 2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 200×40 mm× 10 μm; mobile phase: [water (FA)-ACN];B %: 10%-45%, 8 min) to give N-[2-[8-(1-acetyl-3-piperidyl)-2-[4-(4-methylpiperazin-1-yl)anilino]-7-oxo-pyrido[2,3-d]pyrimidin-6-yl]oxyphenyl]prop-2-enamide (50.2 mg, 68.21 μmol, 48.49% yield, 90.870% purity, FA) as a yellow solid.

### Procedure for Preparation of Compound 008

Compound 008 was prepared as described for the synthesis of compound 007.

### Procedure for Preparation of Compound 009

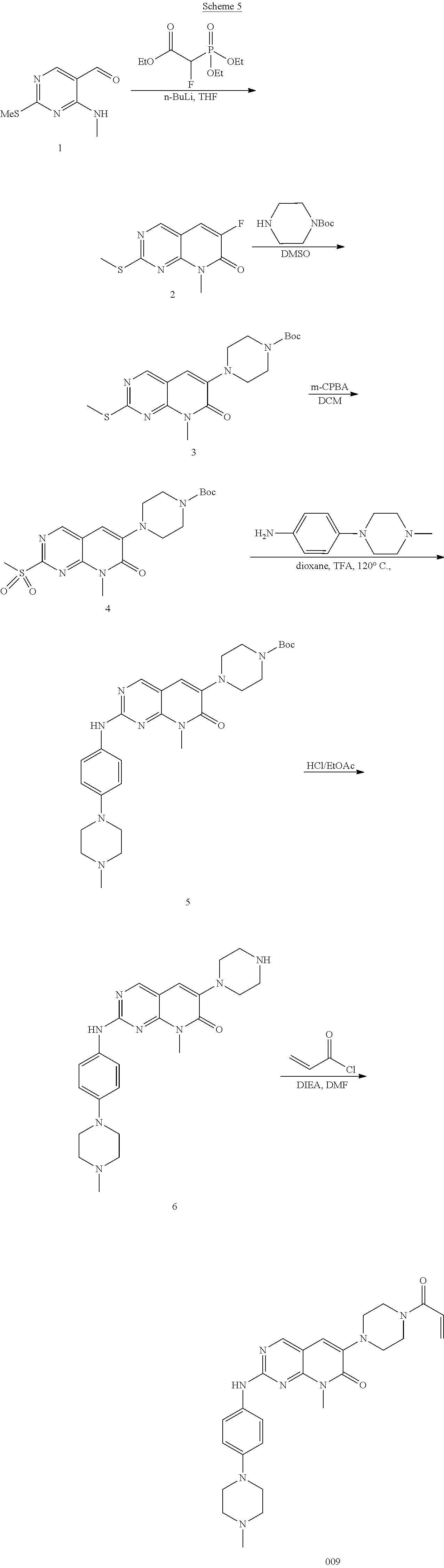

To a solution of ethyl 2-diethoxyphosphoryl-2-fluoro-acetate (3.17 g, 13.10 mmol, 2.67 mL, 2 eq) in THF (24 mL) under a nitrogen atmosphere was added dropwise n-BuLi (2.5 M, 7.86 mL, 3 eq) at −70° C., then the mixture was stirred at −70° C. for 40 min. To this solution was added a solution of 4-(methylamino)-2-methylsulfanyl-pyrimidine-5-carbaldehyde (1.2 g, 6.55 mmol, 1 eq) in THF (8 mL). The mixture was stirred and allowed to warm gradually to 25° C. for 12 hr under nitrogen. LC-MS showed the desired compound was detected. The reaction mixture was quenched by addition NH₄CI (80 mL) at 0° C., extracted with ethyl acetate (80 mL× 2). The combined organic layers were washed with brine (20 mL× 1), dried over Na₂SO₄, filtered and concentrated under reduced pressure to give a residue. The residue was purified by flash silica gel chromatography (ISCOR; 20 g SepaFlash® Silica Flash Column, Eluent of 0˜50% Ethyl acetate/Petroleum ether gradient @ 100 mL/min) to give 6-fluoro-8-methyl-2-methylsulfanyl-pyrido[2,3-d]pyrimidin-7-one (1.2 g, 5.01 mmol, 76.46% yield, 93.99% purity) as a white solid.

To a solution of 6-fluoro-8-methyl-2-methylsulfanyl-pyrido[2,3-d]pyrimidin-7-one (1.2 g, 5.33 mmol, 1 eq) and tert-butyl piperazine-1-carboxylate (1.98 g, 10.66 mmol, 2 eq) in DMSO (20 mL) was added DIEA (1.38 g, 10.66 mmol, 1.86 mL, 2 eq) and K₂CO₃ (2.21 g, 15.98 mmol, 3 eq). The mixture was stirred at 100° C. for 12 hr and 110° C. for 12 hr. LC-MS showed the desired compound was detected. The reaction mixture was partitioned between water (50 mL) and dichloromethane (50 mL× 3). The organic phase was separated, washed with brine (20 mL× 2), dried over Na₂SO₄, filtered and concentrated under reduced pressure to give a residue. The residue was purified by flash silica gel chromatography (ISCO®; 20g SepaFlash® Silica Flash Column, Eluent of 0˜100% Ethyl acetate/Petroleum ether gradient @100 mL/min) to give t tert-butyl 4-(8-methyl-2-methylsulfanyl-7-oxo-pyrido[2,3-d]pyrimidin-6-yl) piperazine-1-carboxylate (1.2 g, 1.72 mmol, 32.35% yield, 56.22% purity) as a light yellow oil.

To a solution of tert-butyl 4-(8-methyl-2-methylsulfanyl-7-oxo-pyrido[2,3-d]pyrimidin-6-yl) piperazine-1-carboxylate (300.00 mg, 766.31 μmol, 1 eq) in DCM (10 mL) was added m-CPBA (280.04 mg, 1.38 mmol, 85% purity, 1.8 eq) at 0° C. The mixture was stirred at 25° C. for 12 hr. LC-MS showed the desired compound was detected. The residue was diluted with Na₂SO₃ (50 mL) and extracted with dichloromethane (50 mL× 3). The combined organic layers were washed with NaHCO₃ (50 mL× 3), dried over Na₂SO₄, filtered and concentrated under reduced pressure to give tert-butyl 4-(8-methyl-2-methylsulfonyl-7-oxo-pyrido[2,3-d]pyrimidin-6-yl) piperazine-1-carboxylate (300 mg, crude) was obtained as a yellow oil.

To a solution of tert-butyl 4-(8-methyl-2-methylsulfonyl-7-oxo-pyrido[2,3-d]pyrimidin-6-yl) piperazine-1-carboxylate (160 mg, 377.82 μmol, 1 eq) in dioxane (2 mL) was added TFA (64.62 mg, 566.73 μmol, 41.96 μL, 1.5 eq) and 4-(4-methylpiperazin-1-yl)aniline (86.72 mg, 453.38 μmol, 1.2 eq). The mixture was stirred at 120° C. for 12 hr. LC-MS showed the desired compound was detected. The reaction mixture was concentrated under reduced pressure to give a residue. The residue was purified by prep-TLC (SiO₂, Dichloromethane: Methanol=10:1) to give tert-butyl 4-[8-methyl-2-[4-(4-methylpiperazin-1-yl)anilino]-7-oxo-pyrido[2,3-d]pyrimidin-6-yl]piperazine-1-carboxylate (90 mg, 157.14 μmol, 41.59% yield, 93.35% purity) as a white solid.

A mixture of tert-butyl 4-[8-methyl-2-[4-(4-methylpiperazin-1-yl)anilino]-7-oxo-pyrido[2,3-d]pyrimidin-6-yl]piperazine-1-carboxylate (90 mg, 168.33 μmol, 1 eq) in HCl/EtOAc (1.5 mL, 4 M) was stirred at 25° C. for 1 hr. LC-MS showed the desired compound was detected. The reaction mixture was concentrated under reduced pressure to give 8-methyl-2-[4-(4-methylpiperazin-1-yl)anilino]-6-piperazin-1-yl-pyrido[2,3-d]pyrimidin-7-one (85 mg, crude, HCl) as a yellow solid.

To a solution of 8-methyl-2-[4-(4-methylpiperazin-1-yl)anilino]-6-piperazin-1-yl-pyrido[2,3-d]pyrimidin-7-one (85 mg, 180.47 μmol, 1 eq, HCl) and DIEA (69.97 mg, 541.40 μmol, 94.30 μL, 3 eq) in DMF (1 mL) was dropwise added a DCM solution of prop-2-enoyl chloride (16.33 mg, 180.47 μmol, 14.72 μL, 1 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 200×40 mm x10 μm; mobile phase: [water (FA)-ACN];B %: 1%-30%, 8 min) to give 8-methyl-2-[4-(4-methylpiperazin-1-yl)anilino]-6-(4-prop-2-enoylpiperazin-1-yl)pyrido[2,3-d]pyrimidin-7-one (14.3 mg, 29.27 μmol, 16.22% yield, 100% purity) as a yellow solid.

### Procedure for Preparation of Compound 010

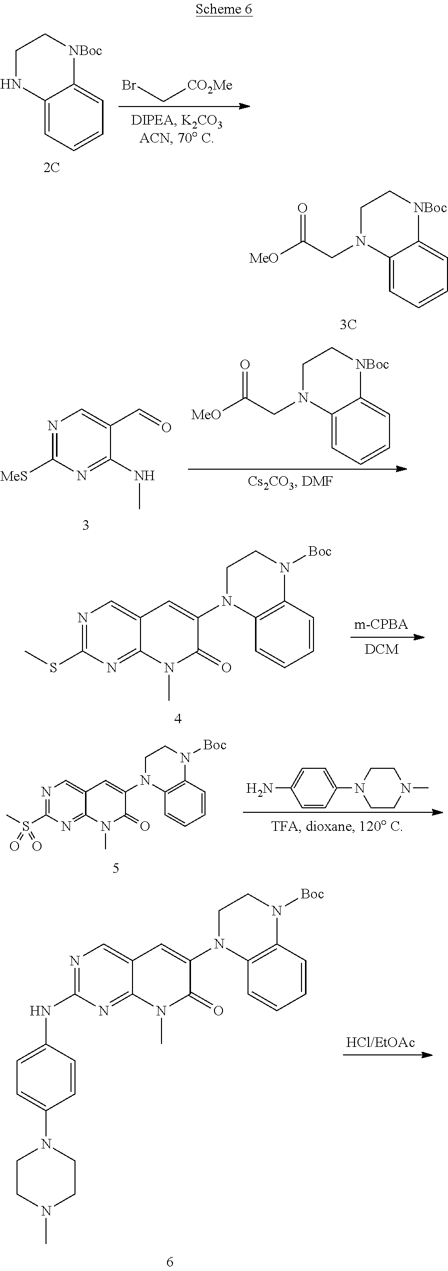

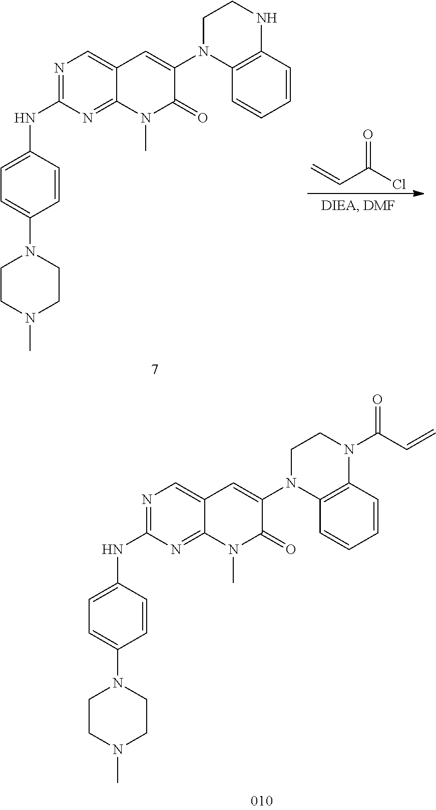

To a solution of tert-butyl 3,4-dihydro-2H-quinoxaline-1-carboxylate (700 mg, 2.9 mmol, 1 eq) and methyl 2-bromoacetate (1.4 g, 8.9 mmol, 846.4 μL, 3 eq) in MeCN (15 mL) was added K₂CO₃ (1.2 g, 8.9 mmol, 3 eq) and DIEA (1.2 g, 8.9 mmol, 1.6 mL, 3 eq). The mixture was stirred at 80° C. for 12 hr. Cs₂CO₃ (1.9 g, 5.9 mmol, 2 eq) and methyl 2-bromoacetate (914.1 mg, 5.9 mmol, 564.3 L, 2 eq) was added to the mixture. The mixture was stirred at 80° C. for 12 hr. LCMS showed consumption of reactant and formation of the desired product mass. The reaction mixture was partitioned between water (50 mL) and ethyl acetate (50 mL× 3). The organic phase was separated, washed with brine (30 mL×1), dried over Na₂SO₄, filtered and concentrated under reduced pressure to give a residue. The residue was purified by prep-HPLC (FA condition) column: Phenomenex luna C18 80×40 mm×3 μm; mobile phase: [water (FA)-ACN];B %: 30%-70%,7 min. to yield tert-butyl 4-(2-methoxy-2-oxo-ethyl)-2,3-dihydroquinoxaline-1-carboxylate (660 mg, 2.2 mmol, 72.1% yield) obtained as a brown oil.

To a solution of 4-(methylamino)-2-methylsulfanyl-pyrimidine-5-carbaldehyde (473.7 mg, 2.6 mmol, 1.2 eq) in DMF (5 mL) was added tert-butyl 4-(2-methoxy-2-oxo-ethyl)-2,3-dihydroquinoxaline-1-carboxylate (660 mg, 2.15 mmol, 1 eq) and Cs₂CO₃ (2.1 g, 6.5 mmol, 3 eq) was stirred at 70° C. for 12 hr. The reaction was monitored by LCMS which showed the reactant was consumed completely and the desired mass peak was detected. The reaction mixture was concentrated under reduced pressure to remove solvent. The residue was diluted with aqueous NaCl (20 mL) and extracted with ethyl acetate (30 mL×3). The combined organic layers were washed with aqueous NaCl (30 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0˜10% Ethyl acetate/Petroleum ether gradient @ 80 mL/min) to yield tert-butyl 4-(8-methyl-2-methylsulfanyl-7-oxo-pyrido[2,3-d]pyrimidin-6-yl)-2,3-dihydroquinoxaline-1-carboxylate (600 mg, 1.4 mmol, 63.4% yield) obtained as a yellow solid.

To a solution of tert-butyl 4-(8-methyl-2-methylsulfanyl-7-oxo-pyrido[2,3-d]pyrimidin-6-yl)-2,3-dihydroquinoxaline-1-carboxylate (520 mg, 1.2 mmol, 1 eq) in DCM (3 mL) was added m-CPBA (480.4 mg, 2.4 mmol, 85% purity, 2 eq) at 0° C. The mixture was stirred at 25° C. for 12 hr. The reaction was monitored by LCMS which showed complete consumption of reactant and detection of the desired mass peak. The reaction mixture was quenched by addition Na₂SO₃ (15 mL) at 0° C., and then extracted with DCM (15 mL× 3). The combined organic layers were dried over Na₂SO₄, filtered and concentrated under reduced pressure to give the product tert-butyl 4-(8-methyl-2-methylsulfonyl-7-oxo-pyrido[2,3-d]pyrimidin-6-yl)-2,3-dihydroquinoxaline-1-carboxylate (600 mg, crude) as yellow oil.

To a solution of 4-(4-methylpiperazin-1-yl)aniline (20.3 mg, 106.0 μmol, 1 eq) in dioxane (2 mL) was added tert-butyl 4-(8-methyl-2-methylsulfonyl-7-oxo-pyrido[2,3-d]pyrimidin-6-yl)-2,3-dihydroquinoxaline-1-carboxylate (50 mg, 106.0 μmol, 1 eq) and TFA (18.1 mg, 159.1 μmol, 11.8 μL, 1.5 eq). And then the mixture was warmed to 120° C. and stirred for 12 hr. The reaction was monitored by LCMS which showed complete consumption of reactant and detection of the desired mass peak. The reaction mixture was concentrated under reduced pressure to remove solvent. The residue was diluted with dimethyl formamide (5 mL) and filtered. The residue was purified by prep-HPLC (FA condition; column: Phenomenex Luna C18 200×40 mm×10 μm; mobile phase: [water (FA)-ACN];B %: 20%-50%, 8 min) to yield tert-butyl 4-[8-methyl-2-[4-(4-methylpiperazin-1-yl)anilino]-7-oxo-pyrido[2,3-d]pyrimidin-6-yl]-2,3-dihydroquinoxaline-1-carboxylate (300 mg) obtained as a brown solid.

To a solution of tert-butyl 4-[8-methyl-2-[4-(4-methylpiperazin-1-yl)anilino]-7-oxo-pyrido[2,3-d]pyrimidin-6-yl]-2, 3-dihydroquinoxaline-1-carboxylate (200 mg, 343.2 μmol, 1 eq) in HCl/EtOAc (4 M, 2 mL, 23.3 eq) was stirred at 25° C. for 1 hr. The reaction was monitored by LCMS which showed complete consumption of reactant and detection of the desired product. The reaction mixture was concentrated under reduced pressure to give the crude product 6-(3,4-dihydro-2H-quinoxalin-1-yl)-8-methyl-2-[4-(4-methylpiperazin-1-yl)anilino]pyrido[2,3-d]pyrimidin-7-one (200 mg, crude) as yellow solid.

To a solution of 6-(3,4-dihydro-2H-quinoxalin-1-yl)-8-methyl-2-[4-(4-methylpiperazin-1-yl)anilino]pyrido[2,3-d]pyrimidin-7-one (100 mg, 192.7 μmol, 1 eq, HCl) and DIEA (74.7 mg, 577.9 μmol, 100.7 μL, 3 eq) in DMF (2 mL) was dropwise added a solution of prop-2-enoyl chloride (20.9 mg, 231.2 μmol, 18.8 μL, 1.2 eq). The mixture was stirred at 0° C. for 1 hr. The reaction was monitored by LCMS which showed complete consumption of reactant and detection of the desired mass peak. The reaction mixture was filtered. The residue was purified by prep-HPLC (FA condition; column: Phenomenex C18 75×30 mm×3 μm; mobile phase: [water (NH₄HCO₃)-ACN];B %: 20%-55%, 8 min) to yield 8-methyl-2-[4-(4-methylpiperazin-1-yl)anilino]-6-(4-prop-2-enoyl-2,3-dihydroquinoxalin-1-yl)pyrido[2,3-d]pyrimidin-7-one (20 mg, 36.5 μmol, 18.9% yield, 97.84% purity) as a yellow solid.

### Procedure for Preparation of Compound 014 and Compound 017

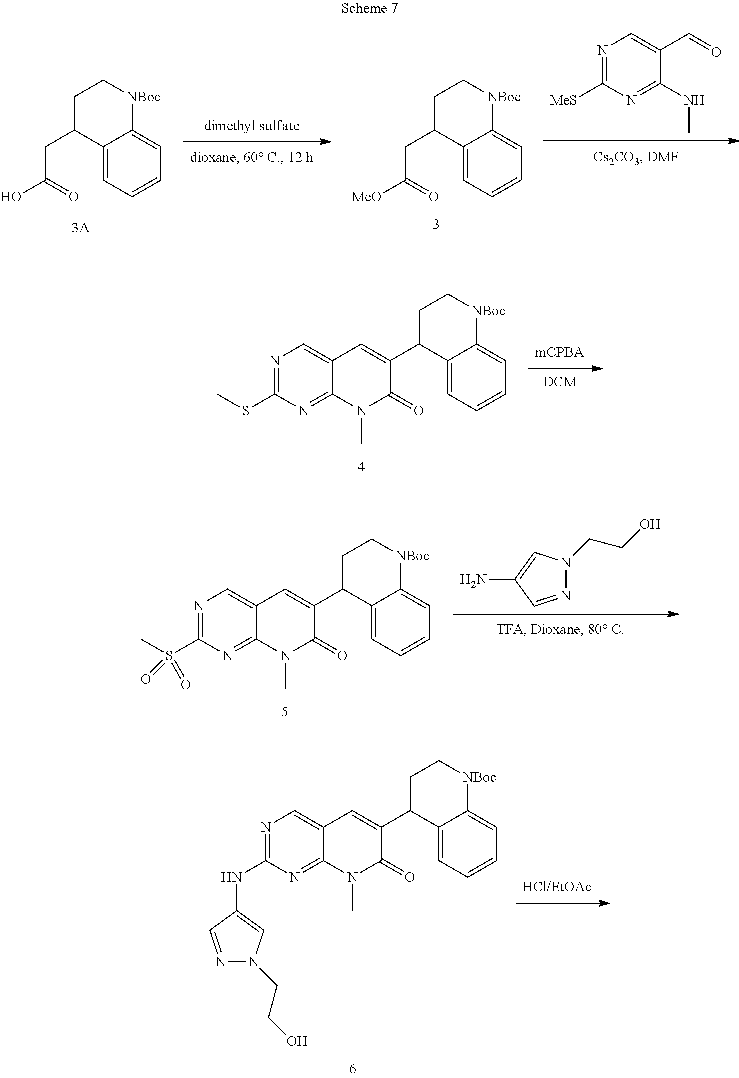

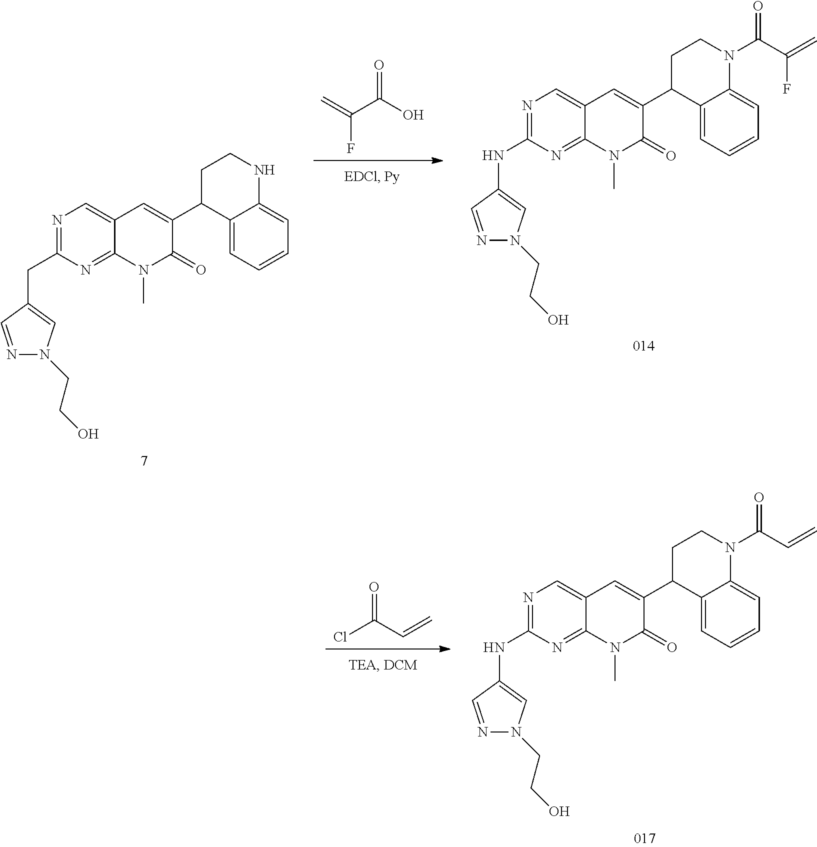

To a solution of 2-(1-tert-butoxycarbonyl-3,4-dihydro-2H-quinolin-4-yl) acetic acid (700 mg, 2.4 mmol, 1 eq) in dioxane (20 mL) was added Cs₂CO₃ (2.3 g, 7.21 mmol, 3 eq) and dimethyl sulfate (519 mg, 4.1 mmol, 390.2 μL, 1.7 eq). The mixture was stirred at 60° C. for 12 hr. LC-MS showed the desired compound was detected. The reaction mixture was partitioned between NaHCO₃ (50 mL) and ethyl acetate (50×3 mL). The organic phase was separated, washed with brine (20 mL× 1), dried over Na₂SO₄, filtered and concentrated under reduced pressure to give a residue. The residue was purified by flash silica gel chromatography (20 g Silica Flash Column, Eluent of 0˜20% Ethyl acetate/Petroleum ether gradient @ 100 mL/min) to give tert-butyl 4-(2-methoxy-2-oxo-ethyl)-3,4-dihydro-2H-quinoline-1-carboxylate (700 mg, 2.3 mmol, 95.4% yield) as a light yellow oil.

To a solution of tert-butyl 4-(2-methoxy-2-oxo-ethyl)-3,4-dihydro-2H-quinoline-1-carboxylate (700 mg, 2.3 mmol, 1 eq) in DMF (10 mL) was added 4-(methylamino)-2-methylsulfanyl-pyrimidine-5-carbaldehyde (420.0 mg, 2.3 mmol, 1 eq) and Cs₂CO₃ (2.2 g, 6.8 mmol, 3 eq). The mixture was stirred at 60° C. for 12 hr. LC-MS showed the desired compound was detected. The reaction mixture was partitioned between H₂O (50 mL) and ethyl acetate (50 mL× 3). The organic phase was separated, washed with brine (50 mL× 2), dried over Na₂SO₄, filtered and concentrated under reduced pressure to give a residue. The residue was purified by flash silica gel chromatography (20 g Silica Flash Column, Eluent of 0˜30% Ethyl acetate/Petroleum ether gradient @ 100 mL/min) to give tert-butyl 4-(8-methyl-2-methylsulfanyl-7-oxo-pyrido[2,3-d]pyrimidin-6-yl)-3,4-dihydro-2H-quinoline-1-carboxylate (750 mg, 1.7 mmol, 74.6% yield) as a light yellow solid.

To a solution of tert-butyl 4-(8-methyl-2-methylsulfanyl-7-oxo-pyrido[2,3-d]pyrimidin-6-yl)-3,4-dihydro-2H-quinoline-1-carboxylate (750 mg, 1.7 mmol, 1 eq) in DCM (20 mL) was added mCPBA (520.8 mg, 2.6 mmol, 85% purity, 1.5 eq) at 0° C. The mixture was stirred at 25° C. for 12 hr. LC-MS showed the desired compound was detected. The reaction mixture was partitioned between Na₂SO₃ (50 mL) and dichloromethane (50×3 mL). The organic phase was separated, washed with NaHCO₃ (50 mL× 3), dried over Na₂SO₄, filtered and concentrated under reduced pressure to give tert-butyl 4-(8-methyl-2-methylsulfonyl-7-oxo-pyrido[2,3-d]pyrimidin-6-yl)-3,4-dihydro-2H-quinoline-1-carboxylate (750 mg, crude) as a yellow solid.

To a solution of tert-butyl 4-(8-methyl-2-methylsulfonyl-7-oxo-pyrido[2,3-d]pyrimidin-6-yl)-3,4-dihydro-2H-quinoline-1-carboxylate (400 mg, 425.0 μmol, 1 eq) in dioxane (4 mL) was added 2-(4-aminopyrazol-1-yl) ethanol (140.5 mg, 552.5 μmol, 1.3 eq) and TFA (155.4 mg, 637.5 μmol, 47.2 μL, 1.5 eq). The mixture was stirred at 80° C. for 12 hr. LC-MS showed the desired compound was detected. The reaction mixture was concentrated under reduced pressure to give a residue. The residue was purified by prep-HPLC (HCl condition, column: Phenomenex luna C18 80×40 mm× 3 μm; mobile phase: [water (HCl)-ACN];B %: 20%-65%,7 min) to give tert-butyl 4-[2-[[1-(2-hydroxyethyl)pyrazol-4-yl]amino]-8-methyl-7-oxo-pyrido[2,3-d]pyrimidin-6-yl]-3,4-dihydro-2H-quinoline-1-carboxylate (300 mg, 579.6 μmol, 68.2% yield) as a light yellow oil.

A mixture of tert-butyl 4-[2-[[1-(2-hydroxyethyl)pyrazol-4-yl]amino]-8-methyl-7-oxo-pyrido[2,3-d]pyrimidin-6-yl]-3,4-dihydro-2H-quinoline-1-carboxylate (300 mg, 579.6 μmol, 1 eq) in HCl/EtOAc (3 mL, 4 M) was stirred at 25° C. for 1 hr. LC-MS showed the desired compound was detected. The reaction mixture was filtered and the filtrate dried in vacuum to give 2-[[1-(2-hydroxyethyl)pyrazol-4-yl]amino]-8-methyl-6-(1,2,3,4-tetrahydroquinolin-4-yl)pyrido[2,3-d]pyrimidin-7-one (300 mg, crude, HCl) as a light yellow solid.

To a solution of 2-[[1-(2-hydroxyethyl)pyrazol-4-yl]amino]-8-methyl-6-(1,2,3,4-tetrahydroquinolin-4-yl)pyrido[2,3-d]pyrimidin-7-one (120 mg, 264.3 μmol, 1 eq, HCl) in Py (3 mL) was added EDCI (76.0 mg, 396.5 μmol, 1.5 eq) and 2-fluoroprop-2-enoic acid (28.5 mg, 317.2 μmol, 1.2 eq). The mixture was stirred at 50° C. for 12 hr. LC-MS showed the desired compound was detected. The reaction mixture was concentrated under reduced pressure to give a residue. The residue was purified by prep-HPLC (TFA condition, column: Phenomenex Luna C18 200×40 mm× 10 μm; mobile phase: [water (FA)-ACN];B %: 25%-55%, 8 min) to give 6-[1-(2-fluoroprop-2-enoyl)-3,4-dihydro-2H-quinolin-4-yl]-2-[[1-(2-hydroxyethyl)pyrazol-4-yl]amino]-8-methyl-pyrido[2,3-d]pyrimidin-7-one (Compound 014) (21.2 mg, 43.3 μmol, 16.3% yield, 100% purity) as a white solid.

To a solution of 2-[[1-(2-hydroxyethyl)pyrazol-4-yl]amino]-8-methyl-6-(1,2,3,4-tetrahydroquinolin-4-yl)pyrido[2,3-d]pyrimidin-7-one (100 mg, 220.3 μmol, 1 eq, HCl) in DCM (4 mL) was added TEA (111.4 mg, 1.1 mmol, 153.3 μL, 5 eq) and prop-2-enoyl chloride (23.9 mg, 264.3 μmol, 21.5 μL, 1.2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 200×40 mm× 10 μm; mobile phase: [water (FA)-ACN];B %: 10%-50%, 8 min) to give 2-[[1-(2-hydroxyethyl)pyrazol-4-yl]amino]-8-methyl-6-(1-prop-2-enoyl-3,4-dihydro-2H-quinolin-4-yl)pyrido[2,3-d]pyrimidin-7-one (Compound 017) (15.5 mg, 32.8 μmol, 14.9% yield, 100% purity) as a white solid.

### Procedure for Preparation of Compound 011

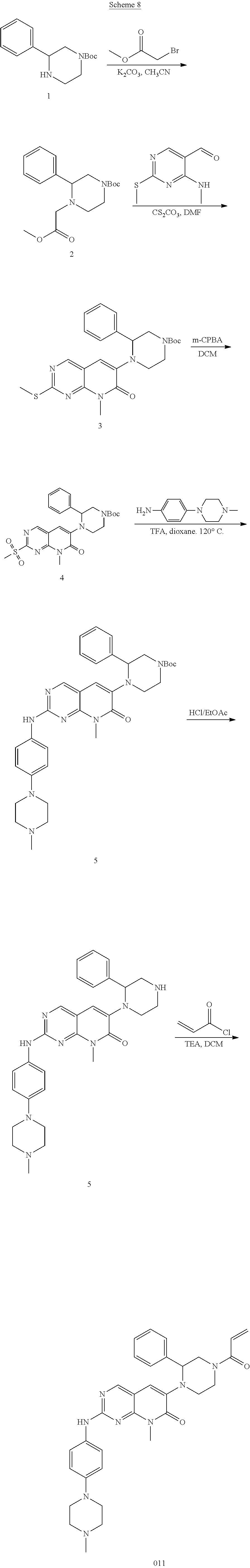

To a solution of tert-butyl 3-phenylpiperazine-1-carboxylate (1.7 g, 6.7 mmol, 1 eq) in CH₃CN (30 mL) was added K₂CO₃ (2.8 g, 19.9 mmol, 3 eq) and methyl 2-bromoacetate (2.0 g, 13.3 mmol, 1.2 mL, 2 eq). The mixture was stirred at 80° C. for 12 hr. LC-MS showed desired compound was detected. The reaction was concentrated under reduce pressure to give a residue. The residue was purified by flash silica gel chromatography (20 g Silica Flash Column, Eluent of 0˜100% Ethyl acetate/Petroleum ether gradient @ 100 mL/min) to give compound tert-butyl 4-(2-methoxy-2-oxo-ethyl)-3-phenyl-piperazine-1-carboxylate (1.5 g, 4.6 mmol, 68.8% yield) as a white solid.

To a solution of tert-butyl 4-(2-methoxy-2-oxo-ethyl)-3-phenyl-piperazine-1-carboxylate (1.5 g, 4.6 mmol, 1 eq) in DMF (18 mL) was added Cs₂CO₃ (4.5 g, 13.7 mmol, 3 eq) and 4-(methylamino)-2-methylsulfanyl-pyrimidine-5-carbaldehyde (1.0 g, 5.5 mmol, 1.2 eq), the mixture was stirred at 110° C. for 12 hr. LC-MS showed desired compound was detected. The reaction mixture was quenched by addition water (10 mL) at 25° C., and then diluted with ethyl acetate (5 mL) and extracted with ethyl acetate (6 mL× 3). The combined organic layers were washed with brine (15× mL), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 80 mL/min) to give compound tert-butyl 4-(8-methyl-2-methylsulfanyl-7-oxo-pyrido[2,3-d]pyrimidin-6-yl)-3-phenyl-piperazine-1-carboxylate (872 mg, 1.9 mmol, 40.9% yield, 100% purity) as a yellow solid.

To a solution of tert-butyl 4-(8-methyl-2-methylsulfanyl-7-oxo-pyrido[2,3-d]pyrimidin-6-yl)-3-phenyl-piperazine-1-carboxylate (372 mg, 795.6 μmol, 1 eq) in DCM (10 mL) was added m-CPBA (306.9 mg, 1.5 mmol, 85% purity, 1.9 eq) at 0° C. Then the mixture was stirred at 25° C. for 12 hr. LC-MS showed reactant was remained, m-CPBA (45.2 mg, 222.8 μmol, 85% purity, 0.28 eq) was added at 0° C., the mixture was stirred at 25° C. for 6 hr. LC-MS showed desired compound was detected. The reaction mixture was quenched by addition sat. Na₂SO₃ (20 mL) at 25° C., then diluted with dichloromethane (15 mL) and extracted with dichloromethane (10 mL×3) and washed with sat·NaHCO₃ (10 mL× 3), dried over Na₂SO₄, filtered and concentrated under reduced pressure to give compound tert-butyl 4-(8-methyl-2-methylsulfinyl-7-oxo-pyrido[2,3-d]pyrimidin-6-yl)-3-phenyl-piperazine-1-carboxylate (400 mg, crude) as a yellow oil.

To a solution of tert-butyl 4-(8-methyl-2-methylsulfinyl-7-oxo-pyrido[2,3-d]pyrimidin-6-yl)-3-phenyl-piperazine-1-carboxylate (260 mg, 483.9 μmol, 90% purity, 1 eq) in dioxane (4 mL) was added 4-(4-methylpiperazin-1-yl)aniline (138.8 mg, 725.8 μmol, 1.5 eq), TFA (82.8 mg, 725.8 μmol, 53.7 μL, 1.5 eq). The mixture was stirred at 120° C. for 12 hr. LC-MS showed reactant was remained, 4-(4-methylpiperazin-1-yl)aniline (92.6 mg, 483.9 μmol, 1 eq) and TFA (27.6 mg, 241.9 μmol, 17.9 μL, 0.5 eq) was added, the mixture was stirred at 120° C. for 12 hr. LC-MS showed desired compound was detected. The reaction was concentrated under the reduce pressure to give a residue. The residue was purified by prep-HPLC (FA condition column: Phenomenex Luna C18 75×30 mm×3 μm; mobile phase: [water (FA)-ACN];B %: 10%-50%, 8 min). to give compound tert-butyl 4-[8-methyl-2-[4-(4-methylpiperazin-1-yl)anilino]-7-oxo-pyrido[2,3-d]pyrimidin-6-yl]-3-phenyl-piperazine-1-carboxylate (170 mg, 264.8 μmol, 54.7% yield, 95.1% purity) as a yellow solid.

Tert-butyl 4-[8-methyl-2-[4-(4-methylpiperazin-1-yl)anilino]-7-oxo-pyrido[2,3-d]pyrimidin-6-yl]-3-phenyl-piperazine-1-carboxylate (170 mg, 278.3 μmol, 1 eq) was added to HCl/EtOAc (5 mL, 4M), the mixture was stirred at 25° C. for 1 hr. LC-MS showed desired compound was detected. The reaction was concentrated under the reduce pressure to give compound 8-methyl-2-[4-(4-methylpiperazin-1-yl)anilino]-6-(2-phenylpiperazin-1-yl)pyrido[2,3-d]pyrimidin-7-one (238 mg, crude) as a yellow oil

To a solution of 8-methyl-2-[4-(4-methylpiperazin-1-yl)anilino]-6-(2-phenylpiperazin-1-yl)pyrido[2,3-d]pyrimidin-7-one (208 mg, 380.2 μmol, 1 eq, HCl) in DCM (2 mL) was added TEA to adjust the pH to 7-8, TEA (115.4 mg, 1.1 mmol, 158.8 μL, 3 eq) was added, then prop-2-enoyl chloride (34.4 mg, 380.2 μmol, 31.0 μL, 1 eq) was added at 0° C., the mixture was stirred at 0° C. for 1 hr. LC-MS showed desired compound was detected. The reaction was concentrated under the reduce pressure. The residue was purified by prep-HPLC (FA condition column: Phenomenex Luna C18 75×30 mm×3 μm; mobile phase: [water (FA)-ACN];B %: 10%-50%, 8 min) to give compound 8-methyl-2-[4-(4-methylpiperazin-1-yl)anilino]-6-(2-phenyl-4-prop-2-enoyl-piperazin-1-yl)pyrido[2,3-d]pyrimidin-7-one (55.3 mg, 96.8 μmol, 25.5% yield, 98.1% purity) as a white solid.

### Procedure for Preparation of Compound 012

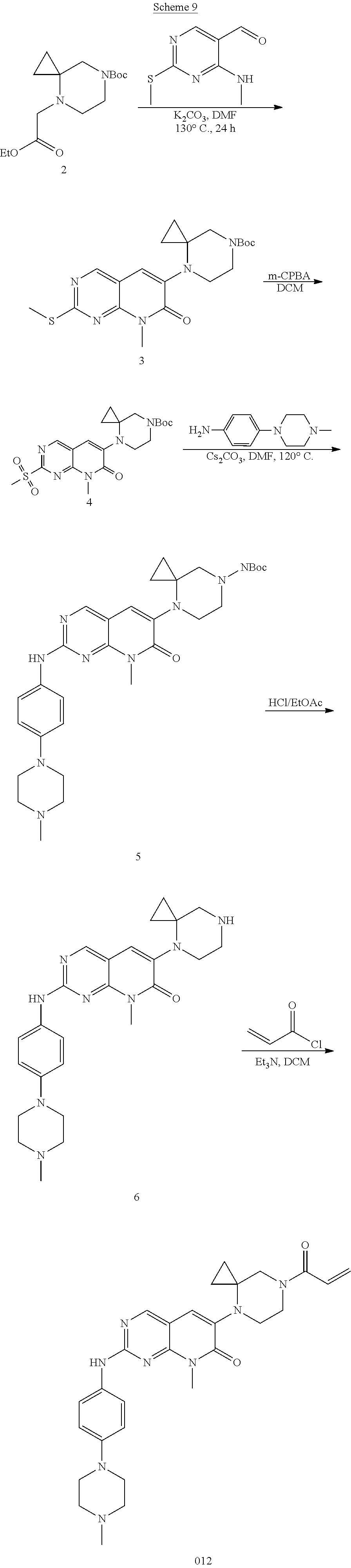

To a solution of 4-(methylamino)-2-methylsulfanyl-pyrimidine-5-carbaldehyde (918.3 mg, 5.0 mmol, 1.5 eq) and tert-butyl 4-(2-methoxy-2-oxo-ethyl)-4,7-diazaspiro[2.5]octane-7-carboxylate (950 mg, 3.3 mmol, 1 eq) in DMF (20 mL) was added K₂CO₃ (923.5 mg, 6.7 mmol, 2 eq). The mixture was stirred at 140° C. for 24 hr under N₂ atmosphere. LCMS showed desired compound was detected. The reaction mixture was diluted with H₂O (60 mL) and extracted with EtOAc (30 mL×3). The combined organic phase was dried with anhydrous Na₂SO₄, the mixture was filtered and the filtrate was concentrated under vacuum. The residue was purified by flash silica gel chromatography (20 g Silica Flash Column, Eluent of 0˜50% Ethyl acetate/Petroleum ether gradient @ 100 mL/min) to give tert-butyl 4-(8-methyl-2-methylsulfanyl-7-oxo-pyrido[2,3-d]pyrimidin-6-yl)-4,7-diazaspiro[2.5]octane-7-carboxylate (1.5 g, 3.0 mmol, 90.3% yield, 84% purity) as a yellow solid

To a solution of tert-butyl 4-(8-methyl-2-methylsulfanyl-7-oxo-pyrido[2,3-d]pyrimidin-6-yl)-4,7-diazaspiro[2.5]octane-7-carboxylate (1.5 g, 2.1 mmol, 57% purity, 1 eq) in DCM (20 mL) was added m-CPBA (1.0 g, 5.1 mmol, 85% purity, 2.5 eq) at 20° C. The mixture was stirred at 20° C. for 2 hr. LC-MS showed desired compound was detected. The reaction mixture was quenched with saturated Na₂SO₃ aqueous solution (20 mL) at 0° C. The reaction mixture was diluted with 10 mL H₂O and extracted with EtOAc (20 mL×3). The combined organic phase was dried with anhydrous Na₂SO₄, the mixture was filtered and the filtrate was concentrated under vacuum. The residue was purified by flash silica gel chromatography (20 g Silica Flash Column, Eluent of 0˜100% Ethyl acetate/Petroleum ether gradient @ 100 mL/min) to give tert-butyl 4-(8-methyl-2-methylsulfonyl-7-oxo-pyrido[2,3-d]pyrimidin-6-yl)-4,7-diazaspiro[2.5]octane-7-carboxylate (580 mg, 1.1 mmol, 54.2% yield, 86% purity) as a yellow solid.

To a solution of tert-butyl 4-(8-methyl-2-methylsulfonyl-7-oxo-pyrido[2,3-d]pyrimidin-6-yl)-4,7-diazaspiro[2.5]octane-7-carboxylate (630 mg, 1.4 mmol, 1 eq) and 4-(4-methylpiperazin-1-yl)aniline (536.1 mg, 2.8 mmol, 2 eq) in DMF (10 mL) was added Cs₂CO₃ (913.3 mg, 2.8 mmol, 2 eq) at 20° C. under N₂ atmosphere. The mixture was stirred at 120° C. for 12 hr under N₂ atmosphere. LC-MS showed desired compound was detected. The mixture was filtered and the filtrate was concentrated under vacuum. The residue was purified by prep-HPLC (FA condition, column: Phenomenex Luna C18 75×30 mm× 3 μm; mobile phase: [water (FA)-ACN];B %: 20%-50%, 8 min) to give tert-butyl 4-[8-methyl-2-[4-(4-methylpiperazin-1-yl)anilino]-7-oxo-pyrido[2,3-d]pyrimidin-6-yl]-4,7-diazaspiro[2.5]octane-7-carboxylate (80 mg, 142.7 μmol, 10.2% yield) as a

A solution of tert-butyl 4-[8-methyl-2-[4-(4-methylpiperazin-1-yl)anilino]-7-oxo-pyrido[2,3-d]pyrimidin-6-yl]-4,7-diazaspiro[2.5]octane-7-carboxylate (70 mg, 124.9 μmol, 1 eq) in HCl/EtOAc (4 mL, 4 M) was stirred at 20° C. for 1 hr. LC-MS showed desired compound was detected. The reaction mixture was concentrated under reduced pressure to remove solvent to give 6-(4,7-diazaspiro[2.5]octan-4-yl)-8-methyl-2-[4-(4-methylpiperazin-1-yl)anilino]pyrido[2,3-d]pyrimidin-7-one (60 mg, crude, HCl) as a yellow solid.

To a solution of 6-(4,7-diazaspiro[2.5]octan-4-yl)-8-methyl-2-[4-(4-methylpiperazin-1-yl)anilino]pyrido[2,3-d]pyrimidin-7-one (60 mg, 130.3 μmol, 1 eq) in DCM (6 mL) was added DIPEA (50.5 mg, 390.8 μmol, 68.1 μL,3 eq) at 0° C. Then prop-2-enoyl chloride (11.8 mg, 130.3 μmol, 10.6 μL, 1 eq) in DCM (0.6 mL) was added to the mixture at 0° C. under N₂ atmosphere.

The mixture was stirred at 0° C. for 1 hr under N₂ atmosphere. LC-MS showed desired compound was detected. The reaction mixture was concentrated under reduced pressure to remove solvent. The residue was purified by prep-HPLC (FA condition, column: Phenomenex Luna C18 75×30 mm× 3 μm; mobile phase: [water (FA)-ACN];B %: 1%-40%, 8 min) to give 8-methyl-2-[4-(4-methylpiperazin-1-yl)anilino]-6-(7-prop-2-enoyl-4,7-diazaspiro[2.5]octan-4-yl)pyrido[2,3-d]pyrimidin-7-one (33.9 mg, 55.2 μmol, 42.4% yield, 91.4% purity, FA) as a white

### Procedure for Preparation of Compound 013

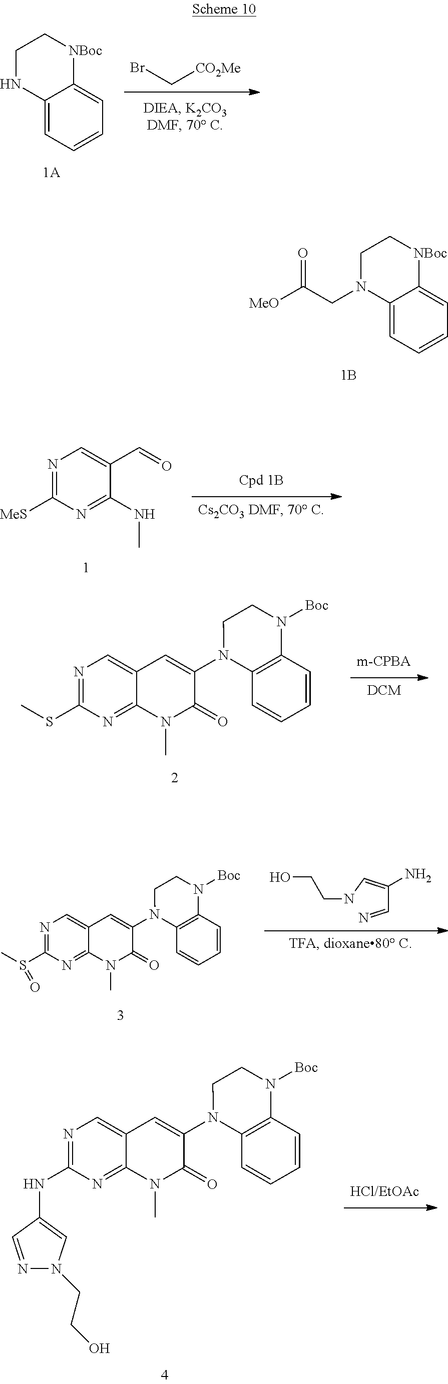

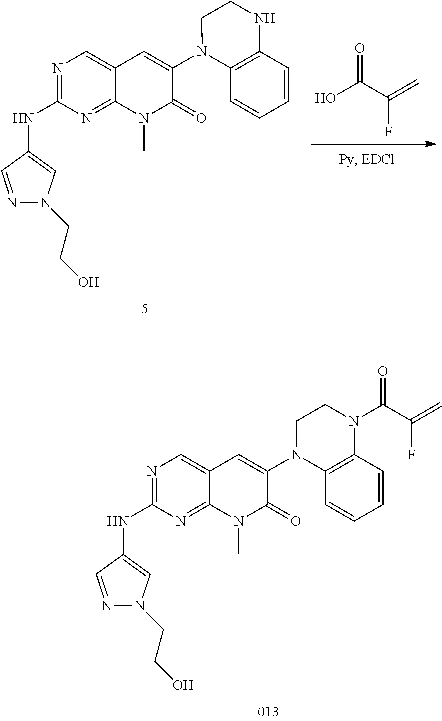

To a solution of tert-butyl 3,4-dihydro-2H-quinoxaline-1-carboxylate (2 g, 8.5 mmol, 1 eq) in DMF (25 mL) was added DIEA (3.3 g, 25.6 mmol, 4.5 mL, 3 eq), methyl 2-bromoacetate (3.9 g, 25.6 mmol, 2.4 mL, 3 eq) and K₂CO₃ (3.5 g, 25.6 mmol, 3 eq). The mixture was stirred at 70° C. for 12 hr. LC-MS showed desired compound was detected. The reaction mixture was quenched by addition H₂O (15 mL) at 25° C., and then diluted with ethyl acetate (10 mL) and extracted with ethyl acetate (15 mL× 3). The combined organic layers were washed with brine (5 mL× 2), dried over Na₂SO₄, filtered and concentrated under reduced pressure to give a residue. The residue was purified by prep-HPLC (FA condition; column: Phenomenex luna C18 (250×70 mm, 15 μm); mobile phase: [water (FA)-ACN];B %: 45%-75%, 20 min) to give compound tert-butyl 4-(2-methoxy-2-oxo-ethyl)-2,3-dihydroquinoxaline-1-carboxylate (1.9 g, 6.2 mmol, 72.7% yield) as a yellow oil.

To a solution of tert-butyl 4-(2-methoxy-2-oxo-ethyl)-2,3-dihydroquinoxaline-1-carboxylate (1.9 g, 6.1 mmol, 1 eq) in DMF (12 mL) was added Cs₂CO₃ (5.9 g, 18.2 mmol, 3 eq) and 4-(methylamino)-2-methylsulfanyl-pyrimidine-5-carbaldehyde (1.3 g, 7.3 mmol, 1.2 eq). The mixture was stirred at 70° C. for 12 hr. LC-MS showed desired compound was detected. The reaction mixture was quenched by addition H₂O (15 mL) at 25° C., and then diluted with ethyl acetate (10 mL) and extracted with ethyl acetate (15 mL× 3). The combined organic layers were washed with brine (5 mL× 2), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 100 mL/min) to give compound tert-butyl 4-(8-methyl-2-methylsulfanyl-7-oxo-pyrido[2,3-d]pyrimidin-6-yl)-2,3-dihydroquinoxaline-1-carboxylate (1.1 g, 2.5 mmol, 40.9% yield) as a white solid.

To a solution of tert-butyl 4-(8-methyl-2-methylsulfanyl-7-oxo-pyrido[2,3-d]pyrimidin-6-yl)-2,3-dihydroquinoxaline-1-carboxylate (1.1 g, 2.4 mmol, 1 eq) in DCM (12 mL) was added m-CPBA (887.9 mg, 4.4 mmol, 85% purity, 1.8 eq) at 0° C., then the mixture was stirred at 25° C. for 12 hr. LC-MS showed desired compound was detected. The reaction mixture was quenched by addition sat. Na₂SO₃ (20 mL) at 25° C., then diluted with dichloromethane (15 mL) and extracted with dichloromethane (10 mL× 3) and washed with sat. NaHCO₃ (10 mL× 3), dried over Na₂SO₄, filtered and concentrated under reduced pressure to give compound tert-butyl 4-(8-methyl-2-methylsulfinyl-7-oxo-pyrido[2,3-d]pyrimidin-6-yl)-2,3-dihydroquinoxaline-1-carboxylate (1.4 g, crude) as a yellow solid.

To a solution of tert-butyl 4-(8-methyl-2-methylsulfinyl-7-oxo-pyrido[2,3-d]pyrimidin-6-yl)-2,3-dihydroquinoxaline-1-carboxylate (200 mg, 439.1 μmol, 1 eq) in dioxane (3 mL) was added 2-(4-aminopyrazol-1-yl) ethanol (66.9 mg, 526.9 μmol, 1.2 eq) and TFA (75.1 mg, 658.6 μmol, 48.8 μL, 1.5 eq). The mixture was stirred at 80° C. for 12 hr (batch×3). LC-MS showed desired compound was detected. The reaction was concentrated under reduce pressure. The residue was purified by prep-HPLC (FA condition column: Phenomenex Luna C18 200×40 mm×10 μm; mobile phase: [water (FA)-ACN];B %: 5%-40%, 8 min) to give compound tert-butyl 4-[2-[[1-(2-hydroxyethyl)pyrazol-4-yl]amino]-8-methyl-7-oxo-pyrido[2,3-d]pyrimidin-6-yl]-2,3-dihydroquinoxaline-1-carboxylate (365 mg, 703.9 μmol, 53.4% yield) as a yellow oil.

Tert-butyl 4-[2-[[1-(2-hydroxyethyl)pyrazol-4-yl]amino]-8-methyl-7-oxo-pyrido[2,3-d]pyrimidin-6-yl]-2,3-dihydroquinoxaline-1-carboxylate (360 mg, 694.2 μmol, 1 eq) was added to HCl/EtOAc (3 mL, 4 M), the mixture was stirred at 20° C. for 2 hr. LC-MS showed desired compound was detected. The reaction was concentrated under reduce pressure to give compound 6-(3,4-dihydro-2H-quinoxalin-1-yl)-2-[[1-(2-hydroxyethyl)pyrazol-4-yl]amino]-8-methyl-pyrido[2,3-d]pyrimidin-7-one (250 mg, crude) as a yellow solid.

To a solution of 6-(3,4-dihydro-2H-quinoxalin-1-yl)-2-[[1-(2-hydroxyethyl)pyrazol-4-yl]amino]-8-methyl-pyrido[2,3-d]pyrimidin-7-one (80 mg, 175.8 μmol, 1 eq, HCl) and 2-fluoroprop-2-enoic acid (19.0 mg, 211.0 μmol, 1.2 eq) in Py (1 mL) was added EDCI (50.6 mg, 263.8 μmol, 1.5 eq). The mixture was stirred at 50° C. for 12 hr. LC-MS showed desired compound was detected. The reaction was concentrated under reduce pressure.to give a residue. The residue was purified by prep-HPLC (FA condition column: Phenomenex Luna C18 75×30 mm× 3 μm; mobile phase: [water (FA)-ACN];B %: 20%-50%, 8 min) to give compound 6-[4-(2-fluoroprop-2-enoyl)-2,3-dihydroquinoxalin-1-yl]-2-[[1-(2-hydroxyethyl)pyrazol-4-yl]amino]-8-methyl-pyrido[2,3-d]pyrimidin-7-one (8 mg, 16.3 μmol, 9.3% yield, 100% purity) as a white solid.

### Procedure for Preparation of Compound 018

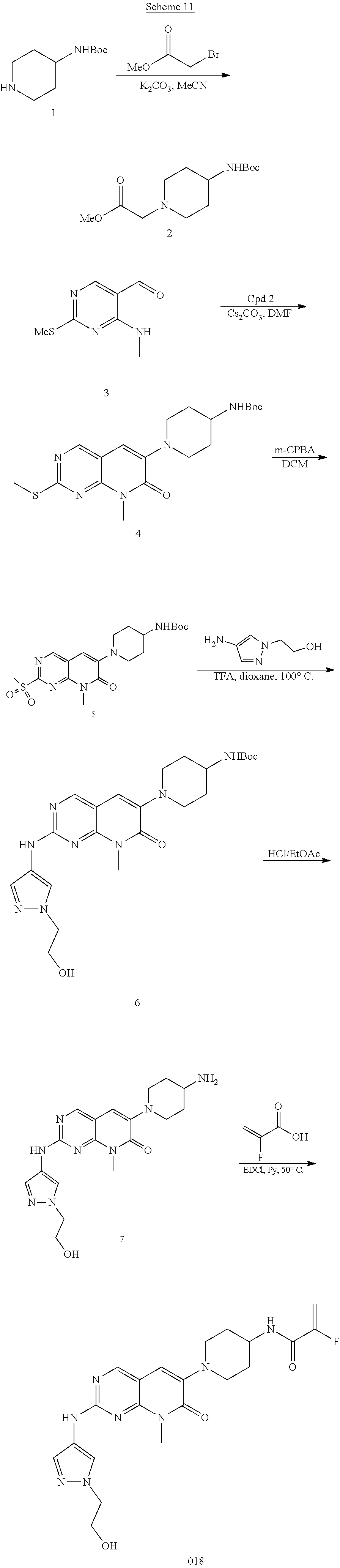

To a solution of tert-butyl N-(4-piperidyl) carbamate (3 g, 15.0 mmol, 1 eq) in MeCN (50 mL) was added K₂CO₃ (4.1 g, 30.0 mmol, 2 eq) and methyl 2-bromoacetate (2.8 g, 18.0 mmol, 1.7 mL, 1.2 eq). The mixture was stirred at 80° C. for 12 hr. LC-MS showed desired compound was detected. The reaction mixture was concentrated under reduced pressure to remove solvent. The residue was diluted with H₂O 50 mL and extracted with ethyl acetate (30 mL× 3). The combined organic layers were washed with brine (20 mL×1), dried over Na₂SO₄, filtered and concentrated under reduced pressure to give methyl 2-[4-(tert-butoxycarbonylamino)-1-piperidyl]acetate (2 g, 7.3 mmol, 49.0% yield) as a yellow solid.

To a solution of methyl 2-[4-(tert-butoxycarbonylamino)-1-piperidyl]acetate (1.8 g, 6.6 mmol, 1 eq) in DMF (20 mL) was added Cs₂CO₃ (6.5 g, 19.8 mmol, 3 eq) and 4-(methylamino)-2-methylsulfanyl-pyrimidine-5-carbaldehyde (1.5 g, 7.9 mmol, 1.2 eq). The mixture was stirred at 100° C. for 12 hr. LC-MS showed desired compound was detected. The residue was diluted with H₂O (120 mL) and extracted with ethyl acetate (130 mL× 3). The combined organic layers were washed with brine (50 mL×1), dried over Na₂SO₄, filtered and concentrated under reduced pressure to give a residue. The residue was purified by flash silica gel chromatography (20 g Silica Flash Column, Eluent of 0˜50% Ethyl acetate/Petroleum ether gradient @ 120 mL/min) to give tert-butyl N-[1-(8-methyl-2-methylsulfanyl-7-oxo-pyrido[2,3-d]pyrimidin-6-yl)-4-piperidyl]carbamate (920 mg, 2.3 mmol, 34.3% yield) as a yellow solid.

To a solution of tert-butyl N-[1-(8-methyl-2-methylsulfanyl-7-oxo-pyrido[2,3-d]pyrimidin-6-yl)-4-piperidyl]carbamate (920 mg, 2.3 mmol, 1 eq) in DCM (10 mL) was added MCPBA (690.9 mg, 3.4 mmol, 85% purity, 1.5 eq) at 0° C. The mixture was stirred at 20° C. for 12 hr. LC-MS showed desired compound was detected. The reaction mixture was partitioned between Na₂SO₃ (100 mL) and dichloromethane (100×3 mL). The organic phase was separated, washed with NaHCO₃ (50 mL×3), dried over Na₂SO₄, filtered and concentrated under reduced pressure to give tert-butyl N-[1-(8-methyl-2-methylsulfonyl-7-oxo-pyrido[2,3-d]pyrimidin-6-yl)-4-piperidyl]carbamate (600 mg, crude) as a yellow solid.

To a solution of tert-butyl N-[1-(8-methyl-2-methylsulfonyl-7-oxo-pyrido[2,3-d]pyrimidin-6-yl)-4-piperidyl]carbamate (250 mg, 571.4 μmol, 1 eq) in dioxane (2 mL) was added TFA (97.7 mg, 857.1 μmol, 63.5 μL, 1.5 eq) and 2-(4-aminopyrazol-1-yl) ethanol (87.2 mg, 685.7 μmol, 1.2 eq). The mixture was stirred at 100° C. for 12 hr. LC-MS showed desired compound was detected. The reaction mixture was concentrated under reduced pressure to remove solvent. The residue was purified by prep-HPLC (FA condition; column: Phenomenex Luna C18 200×40 mm×10 μm; mobile phase: [water (FA)-ACN];B %: 5%-40%, 8 min) to give tert-butyl N-[1-[2-[[1-(2-hydroxyethyl)pyrazol-4-yl]amino]-8-methyl-7-oxo-pyrido[2,3-d]pyrimidin-6-yl]-3,4-dihydro-2H-quinolin-4-yl]carbamate (100 mg, 93.9 μmol, 24.0% yield) as a brown solid.

A mixture of tert-butyl N-[1-[2-[[1-(2-hydroxyethyl)pyrazol-4-yl]amino]-8-methyl-7-oxo-pyrido[2,3-d]pyrimidin-6-yl]-4-piperidyl]carbamate (100 mg, 206.38 μmol, 1 eq) in HCl/EtOAc (1 mL, 4 M) was stirred at 20° C. for 1 hr. LC-MS showed desired compound was detected. The reaction mixture was filtered and concentrated under reduced pressure to give 6-(4-amino-1-piperidyl)-2-[[1-(2-hydroxyethyl)pyrazol-4-yl]amino]-8-methyl-pyrido[2,3-d]pyrimidin-7-one (80 mg, crude) as a brown solid.

To a solution of 6-(4-amino-1-piperidyl)-2-[[1-(2-hydroxyethyl)pyrazol-4-yl]amino]-8-methyl-pyrido[2,3-d]pyrimidin-7-one (80 mg, 208.1 μmol, 1 eq) in Py (1 mL) was added EDCI (59.8 mg, 312.2 μmol, 1.5 eq) and 2-fluoroprop-2-enoic acid (22.5 mg, 249.7 μmol, 1.2 eq). The mixture was stirred at 50° C. for 24 hr. LC-MS showed desired compound was detected. The reaction mixture was filtered and concentrated under reduced pressure to give a residue. The residue was purified by prep-HPLC (FA condition; column: Phenomenex Luna C18 75×30 mm× 3 μm; mobile phase: [water (FA)-ACN];B %: 1%-35%, 8 min) to give 2-fluoro-N-[1-[2-[[1-(2-hydroxyethyl)pyrazol-4-yl]amino]-8-methyl-7-oxo-pyrido[2,3-d]pyrimidin-6-yl]-4-piperidyl]prop-2-enamide (3.3 mg, 7.2 μmol, 3.5% yield, 100% purity) as a brown solid.

### Procedure for Preparation of Compound 015 and Compound 016

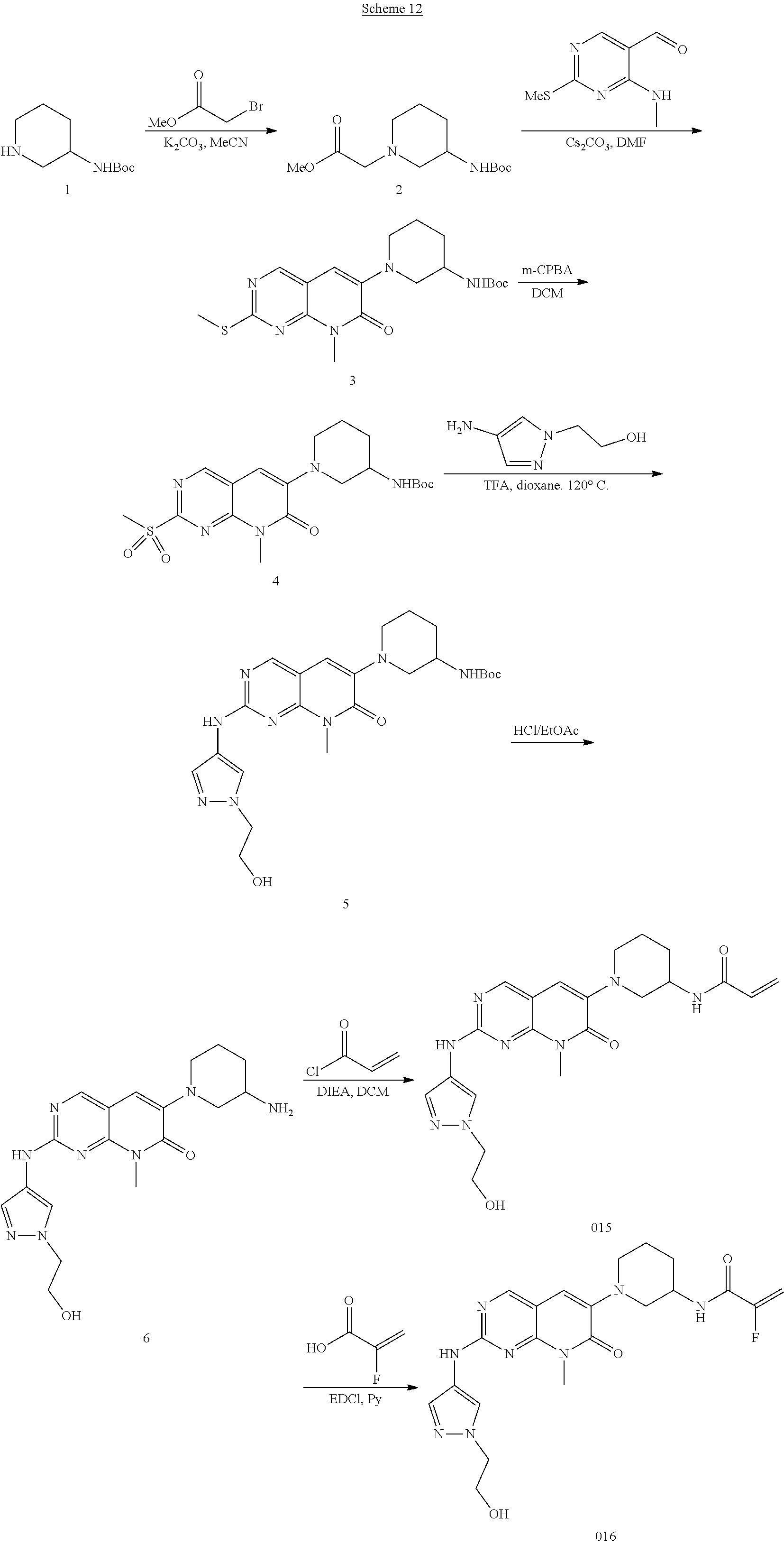

To a solution of tert-butyl N-(3-piperidyl) carbamate (1 g, 5.0 mmol, 1 eq) and methyl 2-bromoacetate (916.6 mg, 6.0 mmol, 565.8 μL, 1.2 eq) in MeCN (60 mL) was added K₂CO₃ (1.4 g, 10.0 mmol, 2 eq) at 20° C. The mixture was stirred at 80° C. for 12 hr. LC-MS showed desired compound was detected. The mixture was filtered and the filtrate was concentrated under vacuum. The residue was purified by flash silica gel chromatography (40 g Silica Flash Column, Eluent of 0˜6% Ethyl acetate/Petroleum ether gradient @ 100 mL/min) to give methyl 2-[3-(tert-butoxycarbonylamino)-1-piperidyl]acetate (4.1 g, 15.1 mmol, 75.4% yield, 100% purity) as a white solid.

To a solution of 4-(methylamino)-2-methylsulfanyl-pyrimidine-5-carbaldehyde (2 g, 10.9 mmol, 1.5 eq) and methyl-2-[3-(tert-butoxycarbonylamino)-1-piperidyl]acetate (2.0 g, 7.3 mmol, 1 eq) in DMF (20 mL) was added Cs₂CO₃ (4.7 g, 14.6 mmol, 2 eq) at 20° C. The mixture was stirred at 90° C. for 12 hr. LC-MS showed desired compound was detected. The reaction mixture was diluted with H₂O (100 mL) and extracted with dichloromethane (70 mL×3). The combined organic phase was dried with anhydrous Na₂SO₄, the mixture was filtered and the filtrate was concentrated under vacuum. The residue was purified by flash silica gel chromatography (40 g Silica Flash Column, Eluent of 0˜100% Ethyl acetate/Petroleum ether gradient @ 100 mL/min) to give tert-butyl N-[1-(8-methyl-2-methylsulfanyl-7-oxo-pyrido[2,3-d]pyrimidin-6-yl)-3-piperidyl]carbamate (86% purity, 3.7 g) as a yellow solid.

To a solution of tert-butyl N-[1-(8-methyl-2-methylsulfanyl-7-oxo-pyrido[2,3-d]pyrimidin-6-yl)-3-piperidyl]carbamate (500 mg, 1.2 mmol, 1 eq) in DCM (20 mL) was added m-CPBA (375.5 mg, 1.85 mmol, 85% purity, 1.5 eq) at 0° C. The mixture was stirred at 20° C. for 3 hr. LC-MS showed desired compound was detected. The reaction mixture was concentrated under reduced pressure to remove solvent to give tert-butyl N-[1-(8-methyl-2-methylsulfinyl-7-oxo-pyrido[2,3-d]pyrimidin-6-yl)-3-piperidyl]carbamate (1.1 g, crude) as a yellow solid.

To a solution of tert-butyl N-[1-(8-methyl-2-methylsulfinyl-7-oxo-pyrido[2,3-d]pyrimidin-6-yl)-3-piperidyl]carbamate (1 g, 2.4 mmol, 1 eq) and 2-(4-aminopyrazol-1-yl) ethanol (603.3 mg, 4.7 mmol, 2 eq) in dioxane (20 mL) was added TFA (405.8 mg, 3.7 mmol, 263.5 μL, 1.5 eq) at 20° C. The mixture was stirred at 120° C. for 12 hr under N₂ atmosphere. LC-MS showed desired compound was detected. The reaction mixture was diluted with H₂O (20 mL) and extracted with ethyl acetate (20 mL× 3). The combined organic phase was dried with anhydrous Na₂SO₄, the mixture was filtered and the filtrate was concentrated under vacuum. The residue was purified by flash silica gel chromatography (40 g Silica Flash Column, Eluent of 0˜9% MeOH/dichloromethane @ 100 mL/min) to give tert-butyl N-[1-[2-[[1-(2-hydroxyethyl)pyrazol-4-yl]amino]-8-methyl-7-oxo-pyrido[2,3-d]pyrimidin-6-yl]-3-piperidyl]carbamate (700 mg, 1.4 mmol, 60.9% yield) as a yellow solid.

A solution of tert-butyl N-[1-[2-[[1-(2-hydroxyethyl)pyrazol-4-yl]amino]-8-methyl-7-oxo-pyrido[2,3-d]pyrimidin-6-yl]-3-piperidyl]carbamate (400 mg, 825.6 μmol, 1 eq) in HCl/EtOAc (8 mL, 4 M) was stirred at 20° C. for 1 hr. LC-MS showed desired compound was detected. The reaction mixture was concentrated under reduced pressure to remove solvent to give 6-(3-amino-1-piperidyl)-2-[[1-(2-hydroxyethyl)pyrazol-4-yl]amino]-8-methyl-pyrido[2,3-d]pyrimidin-7-one (340 mg, crude, HCl) as a yellow solid.

To a solution of 6-(3-amino-1-piperidyl)-2-[[1-(2-hydroxyethyl)pyrazol-4-yl]amino]-8-methyl-pyrido[2,3-d]pyrimidin-7-one (158 mg, 375.4 μmol, 1 eq, HCl) in DCM (3 ml) was added DIEA (145.6 mg, 1.1 mmol, 196.2 μL, 3 eq) at 0° C. The prop-2-enoyl chloride (34.0 mg, 375.4 μmol, 30.6 μL, 1 eq) in DCM (1 mL) was added to the mixture under N₂ atmosphere at 0° C. The mixture was stirred at 0° C. for 1 hr under the N2. LC-MS showed desired compound was detected. The reaction mixture was concentrated under reduced pressure to remove solvent. The residue was purified by prep-HPLC (FA condition, column: Phenomenex Luna C18 200× 40 mm× 10 μm; mobile phase: [water (FA)-ACN];B %: 10%-40%, 8 min) to give N-[1-[2-[[1-(2-hydroxyethyl)pyrazol-4-yl]amino]-8-methyl-7-oxo-pyrido[2,3-d]pyrimidin-6-yl]-3-piperidyl]prop-2-enamide (97.73% purity) (33.4 mg) as a yellow solid.

To a solution of 6-(3-amino-1-piperidyl)-2-[[1-(2-hydroxyethyl)pyrazol-4-yl]amino]-8-methyl-pyrido[2,3-d]pyrimidin-7-one (170 mg, 403.9 μmol, 1 eq, HCl) and 2-fluoroprop-2-enoic acid (54.6 mg, 605.9 μmol, 24.7 L, 1.5 eq) in Py (3 mL) was added EDCI (154.9 mg, 807.8 μmol, 2 eq) at 20° C. The mixture was stirred at 50° C. for 12 hr. LC-MS showed desired compound was detected. The mixture was concentrated under vacuum. The residue was diluted with H₂O (20 mL) and extracted with ethyl acetate (15 mL× 3). The combined organic phase was dried with anhydrous Na₂SO₄, the mixture was filtered and the filtrate was concentrated under vacuum. The residue was purified by prep-HPLC (FA condition, column: Phenomenex Luna C18 75×30 mm× 3 μm; mobile phase: [water (FA)-ACN];B %: 20%-60%, 8 min) to give 2-fluoro-N-[1-[2-[[1-(2-hydroxyethyl)pyrazol-4-yl]amino]-8-methyl-7-oxo-pyrido[2,3-d]pyrimidin-6-yl]-3-piperidyl]prop-2-enamide (8.8 mg, 17.9 μmol, 4.4% yield, 92.7% purity) as a

### Procedure for Preparation of Compound 023

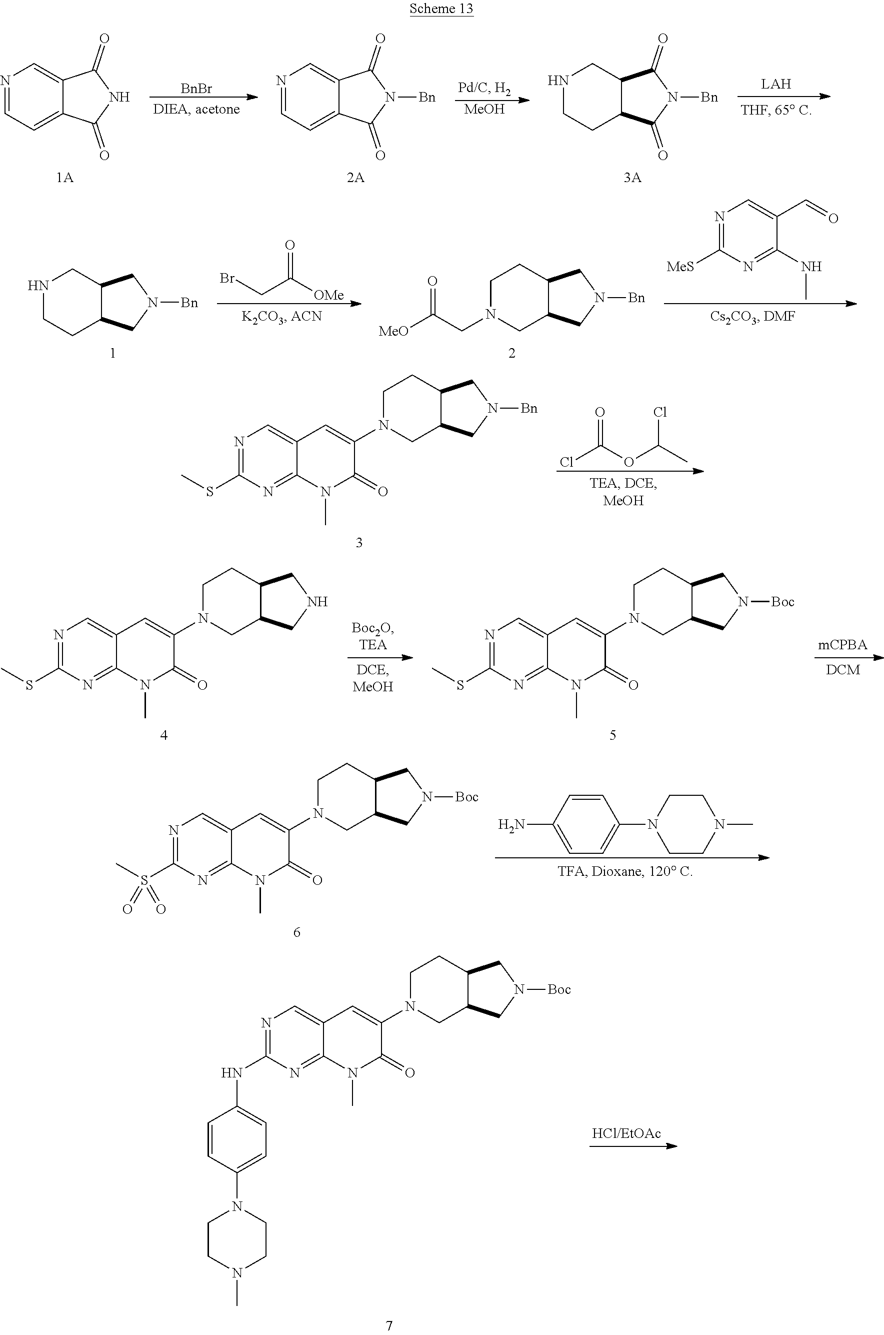

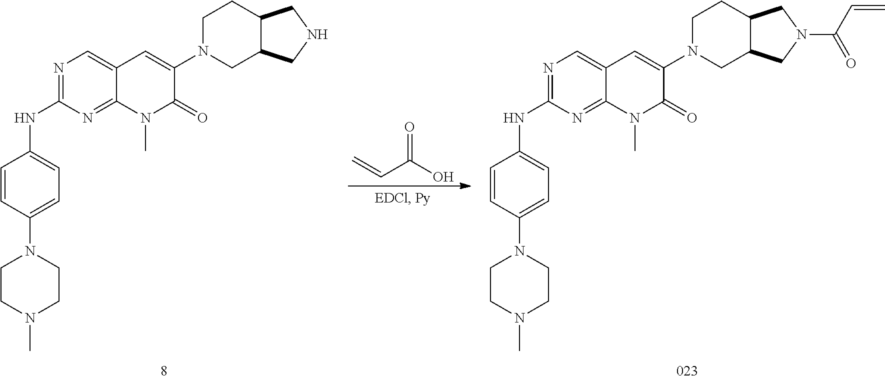

To a solution of pyrrolo[3,4-c]pyridine-1,3-dione (7 g, 47.2 mmol, 1 eq) and DIEA (21.3 g, 165.4 mmol, 28.8 mL, 3.5 eq) in acetone (100 mL) was added bromomethylbenzene (10.5 g, 61.4 mmol, 7.3 mL, 1.3 eq). The mixture was stirred at 60° C. for 5 hr. LC-MS showed desired compound was detected. The residue was diluted with H₂O (300 mL) and extracted with ethyl acetate (900 mL). The combined organic layers were washed with brine (500 mL), dried over Na₂SO₄, filtered and concentrated under reduced pressure to give a residue. The residue was purified by flash silica gel chromatography (80 g Silica Flash Column, Eluent of 0˜50% Ethyl acetate/Petroleum ether gradient @ 100 mL/min) to give 2-benzylpyrrolo[3,4-c]pyridine-1,3-dione (15.5 g, 46.7 mmol, 49.4% yield, 71.9% purity) as a yellow solid.

To a solution of 2-benzylpyrrolo[3,4-c]pyridine-1,3-dione (15.5 g, 65.0 mmol, 1 eq) in MeOH (150 mL) was added Pd/C (10 g, 10% purity) under N₂ atmosphere. The suspension was degassed and purged with H₂ for 3 times. The mixture was stirred under H₂ (15 Psi) at 60° C. for 48 hr. LC-MS showed desired compound was detected. The reaction mixture was filtered and concentrated under reduced pressure to give a residue. The residue was purified by flash silica gel chromatography (40 g Silica Flash Column, Eluent of 0˜100% Ethyl acetate/Petroleum ether gradient @ 120 mL/min) to give 2-benzyl-3a,4,5,6,7,7a-hexahydropyrrolo[3,4-c]pyridine-1,3-dione (11.2 g, 35.1 mmol, 54.0% yield, 76.7% purity) as yellow oil.

To a solution of 2-benzyl-3a,4,5,6,7,7a-hexahydropyrrolo[3,4-c]pyridine-1,3-dione (11 g, 45.0 mmol, 1 eq) in THF (110 mL) was added LAH (5.1 g, 135.0 mmol, 3 eq) at 0° C. The mixture was stirred at 65° C. for 2 hr. LC-MS showed desired compound was detected. The reaction mixture was quenched by addition water (500 mL), 15% NaOH aq. (500 mL) and 1500 mL water at 25° C., and the mixture was filtered to remove the insoluble substance, then organic layers were concentrated under reduced pressure to give a residue. The residue was purified by prep-HPLC (HCl condition; column: Phenomenex luna C18 (250×70 mm, 15 μm); mobile phase: [water (HCl)-ACN];B %: 1%-5%,20 min) to give 2-benzyl-1,3,3a,4,5,6,7,7a-octahydropyrrolo[3,4-c]pyridine (3 g, 13.8 mmol, 30.8% yield) as colorless oil.

To a solution of 2-benzyl-1,3,3a,4,5,6,7,7a-octahydropyrrolo[3,4-c]pyridine (700 mg, 3.2 mmol, 1 eq) in CH₃CN (5 mL) was added K₂CO₃ (1.3 g, 9.7 mmol, 3 eq) and methyl 2-bromoacetate (445.5 mg, 2.9 mmol, 275.0 pL, 0.9 eq). The mixture was stirred at 60° C. for 12 hr. LC-MS showed desired compound was detected. The residue was diluted with H₂O (30 mL) and extracted with ethyl acetate (60 mL). The combined organic layers were washed with brine (20 mL× 3), dried over Na₂SO₄, filtered and concentrated under reduced pressure to give methyl 2-(2-benzyl-3,3a,4,6,7,7a-hexahydro-1H-pyrrolo[3,4-c]pyridin-5-yl)acetate (860 mg, crude) as yellow oil.

To a mixture of methyl 2-(2-benzyl-3,3a,4,6,7,7a-hexahydro-1H-pyrrolo[3,4-c]pyridin-5-yl)acetate (860.0 mg, 2.9 mmol, 1 eq) in DMF (10 mL) was added Cs₂CO₃ (2.9 g, 8.9 mmol, 3 eq) and 4-(methylamino)-2-methylsulfanyl-pyrimidine-5-carbaldehyde (655.7 mg, 3.5 mmol, 1.2 eq), then the mixture was stirred at 70° C. for 12 hr. LC-MS showed desired compound was detected. The reaction mixture was filtered and concentrated under reduced pressure to give a residue. The residue was purified by flash silica gel chromatography (12 g Silica Flash Column, Eluent of 0˜100% Ethyl acetate/Petroleum ether gradient @100 mL/min) to give 6-(2-benzyl-3,3a,4,6,7,7a-hexahydro-1H-pyrrolo[3,4-c]pyridin-5-yl)-8-methyl-2-methylsulfanyl-pyrido[2,3-d]pyrimidin-7-one (1.1 g, 2.2 mmol, 75.2% yield, 86% purity) as yellow oil.

To a mixture of 6-(2-benzyl-3,3a,4,6,7,7a-hexahydro-1H-pyrrolo[3,4-c]pyridin-5-yl)-8-methyl-2-methylsulfanyl-pyrido[2,3-d]pyrimidin-7-one (1 g, 2.3 mmol, 1 eq) in DCE (15 mL) was added TEA (480.0 mg, 4.7 mmol, 660.3 L, 2 eq) and 1-chloroethyl carbonochloridate (1.3 g, 9.4 mmol, 4 eq), then the mixture was stirred at 80° C. for 12 hr. MeOH (15 mL) was added, then the mixture was stirred at 65° C. for 1 hr. LC-MS showed desired compound was detected. Compound 6-(1,2,3,3a,4,6,7,7a-octahydropyrrolo[3,4-c]pyridin-5-yl)-8-methyl-2-methylsulfanyl-pyrido[2,3-d]pyrimidin-7-one (780 mg, 2.3 mmol, 99.2% yield) was obtained as brown oil. The crude product 6-(1,2,3,3a,4,6,7,7a-octahydropyrrolo[3,4-c]pyridin-5-yl)-8-methyl-2-methylsulfanyl-pyrido[2,3-d]pyrimidin-7-one (780 mg, 2.3 mmol, 99.2% yield) was used into the next step without further purification.

To a mixture of 6-(1,2,3,3a,4,6,7,7a-octahydropyrrolo[3,4-c]pyridin-5-yl)-8-methyl-2-methylsulfanyl-pyrido[2,3-d]pyrimidin-7-one (780 mg, 2.3 mmol, 1 eq) in DCE (1 mL) and MeOH (1 mL) was added TEA (1.1 g, 11.7 mmol, 1.6 mL, 5 eq) and Boc₂O (770.4 mg, 3.5 mmol, 810.9 μL, 1.5 eq), then the mixture was stirred at 25° C. for 12 hr. LC-MS showed desired compound was detected. The reaction mixture was concentrated under reduced pressure to remove solvent. The residue was purified by flash silica gel chromatography (12 g Silica Flash Column, Eluent of 0˜100% Ethyl acetate/Petroleum ether gradient @ 100 mL/min) to give tert-butyl 5-(8-methyl-2-methylsulfanyl-7-oxo-pyrido[2,3-d]pyrimidin-6-yl)-3,3a,4,6,7,7a-hexahydro-1H-pyrrolo[3,4-c]pyridine-2-carboxylate (400 mg, 871.2 μmol, 37.0% yield, 94% purity) as colorless oil.

To a solution of tert-butyl 5-(8-methyl-2-methylsulfanyl-7-oxo-pyrido[2,3-d]pyrimidin-6-yl)-3,3a,4,6,7,7a-hexahydro-1H-pyrrolo[3,4-c]pyridine-2-carboxylate (400 mg, 926.8 μmol, 1 eq) in

DCM (5 mL) was added m-CPBA (282.2 mg, 1.3 mmol, 85% purity, 1.5 eq) at 0° C. The mixture was stirred at 20° C. for 1 hr. LC-MS showed desired compound was detected. The reaction mixture was partitioned between Na₂SO₃ (5 mL) and dichloromethane (5×3 mL). The organic phase was separated, washed with NaHCO₃ (5 mL× 3), dried over Na₂SO₄, filtered and concentrated under reduced pressure to give a residue. The residue was purified by prep-TLC (SiO₂, Petroleum ether/Ethyl acetate=0/1) to give tert-butyl 5-(8-methyl-2-methylsulfonyl-7-oxo-pyrido[2,3-d]pyrimidin-6-yl)-3,3a,4,6,7,7a-hexahydro-1H-pyrrolo[3,4-c]pyridine-2-carboxylate (200 mg, 431.4 μmol, 46.5% yield) as yellow oil.

To a solution of tert-butyl 5-(8-methyl-2-methylsulfonyl-7-oxo-pyrido[2,3-d]pyrimidin-6-yl)-3,3a,4,6,7,7a-hexahydro-1H-pyrrolo[3,4-c]pyridine-2-carboxylate (180 mg, 388.3 μmol, 1 eq) in dioxane (0.5 mL) was added 4-(4-methylpiperazin-1-yl)aniline (89.1 mg, 465.9 μmol, 1.2 eq) and TFA (66.4 mg, 582.4 μmol, 43.1 μL, 1.5 eq). The mixture was stirred at 120° C. for 2 hr. LC-MS showed desired mass was detected. The residue was purified by prep-HPLC (FA condition; column: Phenomenex C18 75×30 mm×3 μm; mobile phase: [water (FA)-ACN];gradient: 10%-45% B over 8 min) to give tert-butyl 5-[8-methyl-2-[4-(4-methylpiperazin-1-yl)anilino]-7-oxo-pyrido[2,3-d]pyrimidin-6-yl]-3,3a,4,6,7,7a-hexahydro-1H-pyrrolo[3,4-c]pyridine-2-carboxylate (50 mg, 87.0 μmol, 22.4% yield) as colorless oil.

A mixture of tert-butyl 5-[8-methyl-2-[4-(4-methylpiperazin-1-yl)anilino]-7-oxo-pyrido[2,3-d]pyrimidin-6-yl]-3,3a,4,6,7,7a-hexahydro-1H-pyrrolo[3,4-c]pyridine-2-carboxylate (50 mg, 87.0 μmol, 1 eq) in HCl/EtOAc (1 mL, 4 M) was stirred at 20° C. for 1 hr. LC-MS showed desired compound was detected. The reaction mixture was concentrated under reduced pressure to remove solvent. Compound 6-(1,2,3,3a,4,6,7,7a-octahydropyrrolo[3,4-c]pyridin-5-yl)-8-methyl-2-[4-(4-methylpiperazin-1-yl)anilino]pyrido[2,3-d]pyrimidin-7-one (67 mg, crude) was obtained as a yellow solid.

To a solution of 6-(1,2,3,3a,4,6,7,7a-octahydropyrrolo[3,4-c]pyridin-5-yl)-8-methyl-2-[4-(4-methylpiperazin-1-yl)anilino]pyrido[2,3-d]pyrimidin-7-one (32 mg, 62.6 μmol, 1 eq, HCl) in Py (1 mL) was added EDCI (18.0 mg, 93.9 μmol, 1.5 eq) and acrylic acid (5.4 mg, 75.1 μmol, 5.1 μL, 1.2 eq). The mixture was stirred at 50° C. for 12 hr. LC-MS showed desired compound was detected. The residue was purified by prep-HPLC (FA condition, column: Phenomenex Luna C18 200×40 mm× 10 μm; mobile phase: [water (FA)-ACN];B %: 1%-30%, 8 min) to give 8-methyl-2-[4-(4-methylpiperazin-1-yl)anilino]-6-(2-prop-2-enoyl-3,3a,4,6,7,7a-hexahydro-1H-pyrrolo[3,4-c]pyridin-5-yl)pyrido[2,3-d]pyrimidin-7-one (2.7 mg, 5.2 μmol, 8.3% yield, 100% purity) as a yellow solid.

### Procedure for Preparation of Compound 020

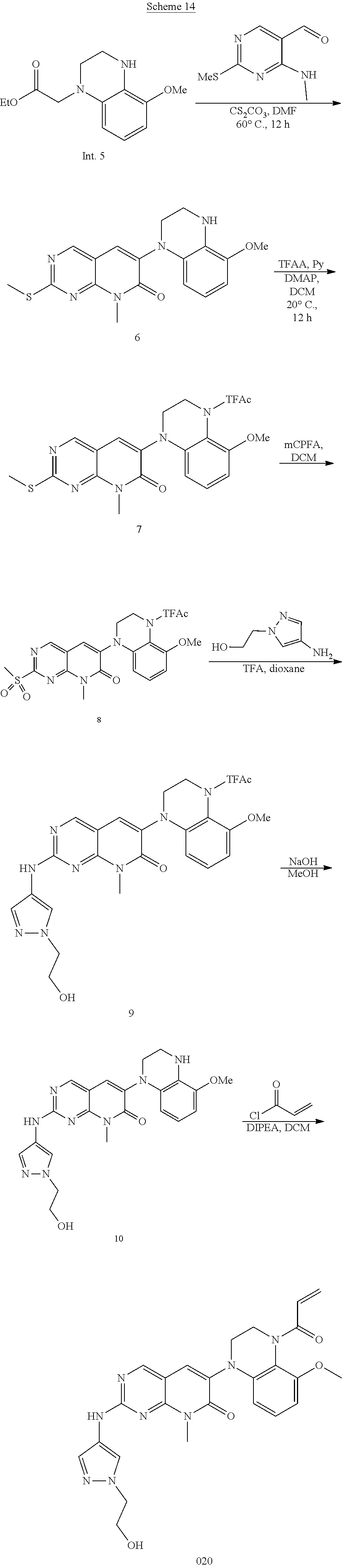

To a solution of ethyl 2-(5-methoxy-3,4-dihydro-2H-quinoxalin-1-yl)acetate (1.2 g, 4.8 mmol, 1 eq) in DMF (20 mL) was added Cs₂CO₃ (3.1 g, 9.6 mmol, 2 eq) and 4-(methylamino)-2-methylsulfanyl-pyrimidine-5-carbaldehyde (1.3 g, 7.2 mmol, 1.5 eq) at 20° C. The mixture was stirred at 60° C. for 12 hr. LC-MS showed desired compound was detected. The reaction mixture was diluted with H₂O (20 mL) and extracted with EtOAc (15 mL×3). The combined organic phase was dried with anhydrous Na₂SO₄, the mixture was filtered and the filtrate was concentrated under vacuum. The residue was purified by prep-TLC (SiO₂, Petroleum ether/Ethyl acetate=1/1) to give 6-(5-methoxy-3,4-dihydro-2H-quinoxalin-1-yl)-8-methyl-2-methylsulfanyl-pyrido[2,3-d]pyrimidin-7-one (1.2 g, 3.2 mmol, 67.2% yield, 96% purity) as a

To a solution of 6-(5-methoxy-3,4-dihydro-2H-quinoxalin-1-yl)-8-methyl-2-methylsulfanyl-pyrido[2,3-d]pyrimidin7-one (1.2 g, 3.4 mmol, 1 eq) in DCM (20 mL) was added DMAP (41.0 mg, 335.6 μmol, 0.1 eq), Py (796.5 mg, 10.1 mmol, 812.7 μL, 3 eq) TFAA (1.8 g, 8.4 mmol, 1.2 mL, 2.5 eq) at 0° C. The mixture was stirred at 20° C. for 12 hr. LC-MS showed desired compound was detected. The reaction mixture was diluted with H₂O (20 mL) and extracted with DCM (15 mL× 3). The combined organic phase was dried with anhydrous Na₂SO₄, the mixture was filtered and the filtrate was concentrated under vacuum. The residue was purified by flash silica gel chromatography (12 g Silica Flash Column, Eluent of 0˜13% Ethyl acetate/Petroleum ether gradient @ 100 mL/min) to give 6-[5-methoxy-4-(2,2,2-trifluoroacetyl)-2,3-dihydroquinoxalin-1-yl]-8-methyl-2-methylsulfanylpyrido[2,3-d]pyrimidin-7-one (1.4 g) as a yellow solid.

To a solution of 6-[5-methoxy-4-(2,2,2-trifluoroacetyl)-2,3-dihydroquinoxalin-1-yl]-8-methyl-2-methylsulfanylpyrido[2,3-d]pyrimidin-7-one (1.4 g, 3.0 mmol, 1 eq) in DCM (2 mL) was added mCPBA (1.2 g, 6.0 mmol, 85% purity, 2 eq) at 0° C. The mixture was stirred at 20° C. for 12 hr. LC-MS showed desired compound was detected. The reaction mixture was quenched with saturated Na₂SO₃ aqueous solution (20 mL) at 0° C., the reaction mixture was diluted with H₂O (10 mL) and extracted with EtOAc (15 mL× 3). The combined organic phase was dried with anhydrous Na₂SO₄, the mixture was filtered and the filtrate was concentrated under vacuum to give 6-[5-methoxy-4-(2,2,2-trifluoroacetyl)-2,3-dihydroquinoxalin-1-yl]-8-methyl-2-methylsulfonylpyrido[2,3-d]pyrimidin-7-one (1.5 g, crude) as a yellow solid.

To a solution of 6-[5-methoxy-4-(2,2,2-trifluoroacetyl)-2,3-dihydroquinoxalin-1-yl]-8-methyl-2-methylsulfonylpyrido[2,3-d]pyrimidin-7-one (370 mg, 743.8 μmol, 1 eq) and 2-(4-aminopyrazol-1-yl) ethanol (189.1 mg, 1.5 mmol, 2 eq) in dioxane (3 mL) was added TFA (127.2 mg, 1.1 mmol, 82.6 μL, 1.5 eq) at 20° C. The mixture was stirred at 80° C. for 12 hr. LC-MS showed desired compound was detected. The reaction mixture was diluted with H₂O (20 mL) and extracted with EtOAc (15 mL×3). The combined organic phase was dried with anhydrous Na₂SO₄, the mixture was filtered and the filtrate was concentrated under vacuum. The residue was purified by flash silica gel chromatography (12 g Silica Flash Column, Eluent of 0˜8% MeOH/DCM @ 100 mL/min) to give 2-[[1-(2-hydroxyethyl)pyrazol-4-yl]amino]-6-[5-methoxy-4-(2,2,2-trifluoroacetyl)-2,3-dihydroquinoxalin1-yl]-8-methyl-pyrido[2,3-d]pyrimidin-7-one (350 mg, 642.8 μmol, 86.4% yield) as a yellow solid.

To a solution of 2-[[1-(2-hydroxyethyl)pyrazol-4-yl]amino]-6-[5-methoxy-4-(2,2,2-trifluoroacetyl)-2,3-dihydroquinoxalin-1-yl]-8-methyl-pyrido[2,3-d]pyrimidin-7-one (250 mg, 459.2 μmol, 1 eq) in MeOH (4 mL) was added NaOH(1 M, 4 mL, 8.71 eq) at 20° C. The mixture was stirred at 20° C. for 12 hr. LC-MS showed desired compound was detected. The reaction mixture was diluted with H₂O (30 mL) and extracted with EtOAc (20 mL×3). The combined organic phase was dried with anhydrous Na₂SO₄, the mixture was filtered and the filtrate was concentrated under vacuum. The residue was purified by flash silica gel chromatography (12 g Silica Flash Column, Eluent of 0˜100% MeOH/DCM @ 100 mL/min) to give 2-[[1-(2-hydroxyethyl)pyrazol-4-yl]amino]-6-(5-methoxy-3,4-dihydro-2H-quinoxalin-1-yl)-8-methylpyrido[2,3-d]pyrimidin-7-one (220 mg) as a yellow solid.

To a solution of 2-[[1-(2-hydroxyethyl)pyrazol-4-yl]amino]-6-(5-methoxy-3,4-dihydro-2H-quinoxalin-1-yl)-8-methylpyrido[2,3-d]pyrimidin-7-one (200 mg, 446.0 μmol, 1 eq) in DCM (1 mL) was added DIPEA (172.9 mg, 1.3 mmol, 233.0 μL, 3 eq) and prop-2-enoyl chloride (40.4 mg, 446.0 μmol, 36.4 μL, 1 eq) at 0° C. The mixture was stirred at 20° C. for 1 hr. The reaction mixture was concentrated under reduced pressure to remove solvent. The residue was purified by prep-HPLC (FA condition, column: Phenomenex Luna C18 75×30 mm×3 μm; mobile phase: [water (FA)-ACN];B %: 15%-45%, 8 min) to give 2-[[1-(2-hydroxyethyl)pyrazol-4-yl]amino]-6-(5-methoxy-4-prop-2-enoyl-2,3-dihydroquinoxalin-1-yl)-8-methyl-pyrido[2,3-d]pyrimidin-7-one (50.02 mg, 98.21 μmol, 22.02% yield, 98.67% purity) as a yellow solid.

### Procedure for Preparation of Compound 019

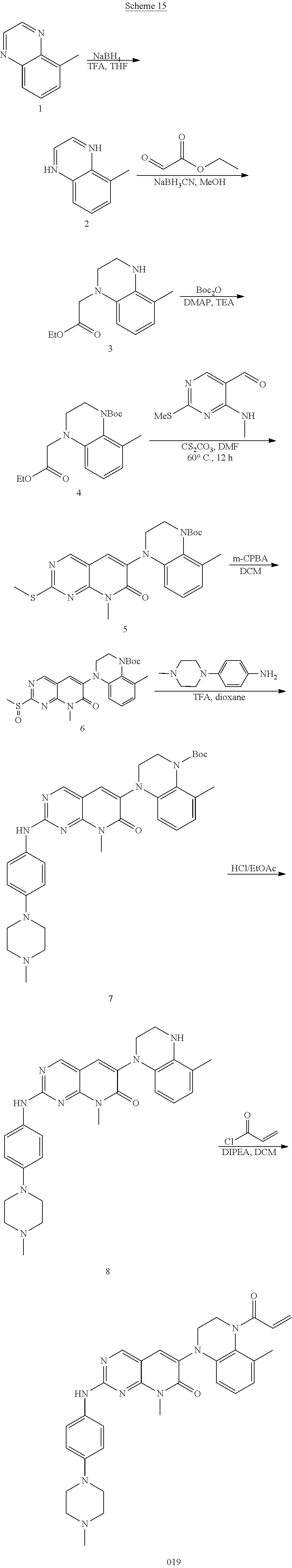

To a solution of 5-methylquinoxaline (5.0 g, 34.7 mmol, 1 eq) in THF (50 mL) was added NaBH₄ (4.6 g, 121.4 mmol, 3.5 eq). The mixture was stirred at 0° C. for 0.5 hr. TFA (7.1 g, 62.4 mmol, 4.6 mL, 1.8 eq) was added to the mixture at 0° C. The mixture was stirred at 0° C. for 0.5 hr. Then the mixture was stirred at 20° C. for 12 hr. LC-MS showed desired compound was detected. The reaction mixture was added water (100 mL) at 0° C. The mixture was extracted with ethyl acetate (20 mL× 3). The combined organic layers were washed with brine (15 mL×2), dried over Na₂SO₄, filtered and concentrated under reduced pressure to give a residue. The residue was purified by column chromatography (SiO₂, Petroleum ether/Ethyl acetate=5/1 to 3/1) to give 5-methyl-1,2,3,4-tetrahydroquinoxaline (4.7 g, 31.7 mmol, 91.4% yield) as red oil.

To a solution of 5-methyl-1,2,3,4-tetrahydroquinoxaline (4.3 g, 29.0 mmol, 1 eq) and ethyl 2-oxoacetate (6.5 g, 31.9 mmol, 50% purity, 1.1 eq) in MeOH (100 mL) was adjust pH to 4-5 by AcOH, the mixture was stirred at 25° C. for 3 hr, then NaBH₃CN (5.5 g, 87.0 mmol, 3 eq) was added, the mixture was stirred at 25° C. for 12 hr. LC-MS showed desired compound was detected. The mixture was concentrated to get a residue, the residue was quenched by Sat. NaHCO₃ (20 mL) and then extracted by ethyl acetate (30 mL×3), the combined organic layers was dried by Na₂SO₄, filtered and the filtrate was concentrated to get a residue. The residue was purified by column chromatography (SiO₂, Petroleum ether/Ethyl acetate=10/1 to 3/1) to give ethyl 2-(5-methyl-3,4-dihydro-2H-quinoxalin-1-yl)acetate (3 g, 12.8 mmol, 44.1% yield) as brown oil.

To a solution of ethyl 2-(5-methyl-3,4-dihydro-2H-quinoxalin-1-yl)acetate (2.5 g, 10.7 mmol, 1 eq) in Boc₂O (15 mL) was added DMAP (130.4 mg, 1.1 mmol, 0.1 eq) and TEA (5.4 g, 53.4 mmol, 7.4 mL, 5 eq). The mixture was stirred at 80° C. for 48 hr. LC-MS showed desired compound was detected. The mixture was concentrated to get a residue. The residue was purified by column chromatography (SiO₂, Petroleum ether/Ethyl acetate=10/1 to 5/1) to give tert-butyl 4-(2-ethoxy-2-oxo-ethyl)-8-methyl-2,3-dihydroquinoxaline-1-carboxylate (2.45 g, 7.3 mmol, 68.7% yield) as red oil.

To a solution of tert-butyl 4-(2-ethoxy-2-oxo-ethyl)-8-methyl-2,3-dihydroquinoxaline-1-carboxylate (2.4 g, 7.3 mmol, 1 eq) in DMF (20 mL) was added Cs₂CO₃ (7.2 g, 22.0 mmol, 3 eq) and 4-(methylamino)-2-methylsulfanyl-pyrimidine-5-carbaldehyde (1.5 g, 8.1 mmol, 1.1 eq). The mixture was stirred at 80° C. for 12 hr. LC-MS showed desired compound was detected. The mixture was poured into water (100 mL) and then extracted by ethyl acetate (30 ml×3), the organic layers was washed by brine (20 mL×2), dried by Na₂SO₄, then filtered and the filtrate was concentrated to get a residue. The residue was purified by flash silica gel chromatography (25 g Silica Flash Column, Eluent of 0˜15% Ethyl acetate/Petroleum ether gradient @ 120 mL/min) to give tert-butyl 8-methyl-4-(8-methyl-2-methylsulfanyl-7-oxo-pyrido[2,3-d]pyrimidin-6-yl)-2,3-dihydroquinoxaline-1-carboxylate (2.1 g, 4.6 mmol, 63.2% yield) as a yellow solid.

To a solution of tert-butyl 8-methyl-4-(8-methyl-2-methylsulfanyl-7-oxo-pyrido[2,3-d]pyrimidin-6-yl)-2,3-dihydroquinoxaline-1-carboxylate (2.1 g, 4.63 mmol, 1 eq) in DCM (50 mL) was added m-CPBA (1.7 g, 8.3 mmol, 85% purity, 1.8 eq). The mixture was stirred at 25° C. for 12 hr. LC-MS showed desired compound was detected. The reaction mixture was partitioned between Na₂SO₃ (30 mL) and dichloromethane (30 mL× 3). The organic phase was separated, washed with NaHCO₃ (20 mL× 3), dried over Na₂SO₄, filtered and concentrated under reduced pressure to give a residue. The residue was purified by column chromatography (SiO₂, Petroleum ether/Ethyl acetate=10/1 to 1/1) to give compound tert-butyl 8-methyl-4-(8-methyl-2-methylsulfinyl-7-oxo-pyrido[2,3-d]pyrimidin-6-yl)-2,3-dihydroquinoxaline-1-carboxylate (1 g, 2.1 mmol, 46.0% yield) as a red solid.

To a solution of tert-butyl 8-methyl-4-(8-methyl-2-methylsulfinyl-7-oxo-pyrido[2,3-d]pyrimidin-6-yl)-2,3-dihydroquinoxaline-1-carboxylate (400 mg, 857.8 μmol, 1 eq) in dioxane (4 mL) was added TFA (116.6 mg, 1022.2 μmol, 75.9 μL, 1.2 eq) and 4-(4-methylpiperazin-1-yl)aniline (163.7 mg, 851.9 μmol, 1 eq). The mixture was stirred at 80° C. for 12 hr. LC-MS showed desired compound was detected. The mixture was concentrated to get a residue. The residue was purified by prep-HPLC (FA condition; column: Phenomenex luna C18 100×40 mm× 3 μm; mobile phase: [water (TFA)-ACN];B %: 25%-70%, 8 min) to give tert-butyl 8-methyl-4-[8-methyl-2-[4-(4-methylpiperazin-1-yl)anilino]-7-oxo-pyrido[2,3-d]pyrimidin-6-yl]-2,3-dihydroquinoxaline-1-carboxylate (300 mg, 502.8 μmol, 59.0% yield) as a red solid.

To a solution of tert-butyl 8-methyl-4-[8-methyl-2-[4-(4-methylpiperazin-1-yl)anilino]-7-oxo-pyrido[2,3-d]pyrimidin-6-yl]-2,3-dihydroquinoxaline-1-carboxylate (300 mg, 502.8 μmol, 1 eq) in EtOAc (5 mL) was added HCl/EtOAc (10 mL, 4M). The mixture was stirred at 25° C. for 1 hr. LC-MS showed desired compound was detected. The mixture was concentrated to give 8-methyl-6-(5-methyl-3,4-dihydro-2H-quinoxalin-1-yl)-2-[4-(4-methylpiperazin-1-yl)anilino]pyrido[2,3-d]pyrimidin-7-one (250 mg, crude) as a yellow solid.

To a solution of 8-methyl-6-(5-methyl-3,4-dihydro-2H-quinoxalin-1-yl)-2-[4-(4-methylpiperazin-1-yl)anilino]pyrido[2,3-d]pyrimidin-7-one (100 mg, 187.6 μmol, 1 eq, HCl) in DCM (1 mL) was added DIEA (97.0 mg, 750.4 μmol, 130.7 μL, 4 eq) and prop-2-enoyl chloride (17.0 mg, 187.6 μmol, 15.3 μL, 1 eq). The mixture was stirred at 0° C. for 1 hr. LC-MS showed desired compound was detected. The mixture was concentrated to get a residue. The residue was purified by prep-HPLC (FA condition; column: Phenomenex Luna C18 200×40 mm×μm; mobile phase: [water (FA)-ACN];B %: 5%-40%, 8 min) to give compound 8-methyl-2-[4-(4-methylpiperazin-1-yl)anilino]-6-(5-methyl-4-prop-2-enoyl-2,3-dihydroquinoxalin-1-yl)pyrido[2,3-d]pyrimidin-7-one (28.1 mg, 51.0 μmol, 27.2% yield, 100% purity) as a white solid.

### Procedure for Preparation of Compound 022

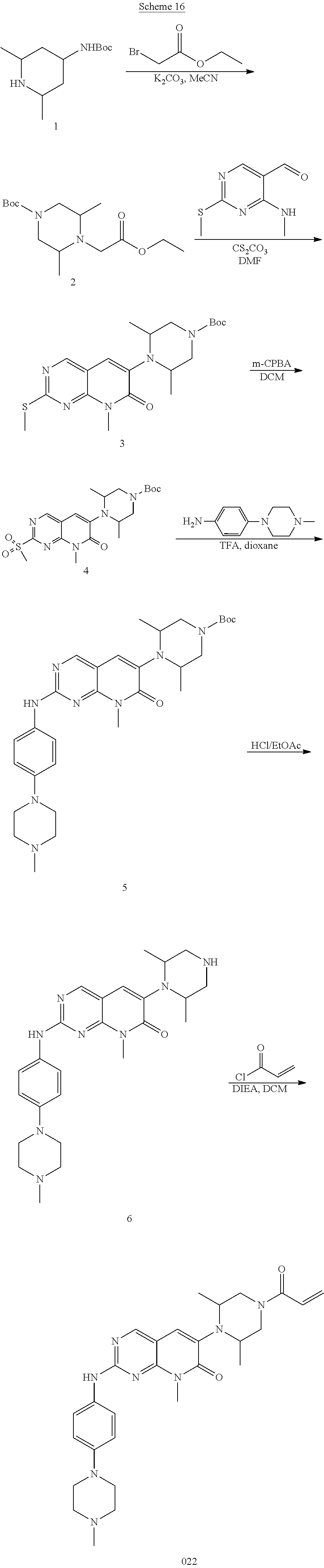

To a solution of tert-butyl 3,5-dimethylpiperazine-1-carboxylate (1 g, 4.67 mmol, 1 eq) in MeCN (10 mL) was added K₂CO₃ (1.3 g, 9.3 mmol, 2 eq) and methyl 2-bromoacetate (856.6 mg, 5.6 mmol, 528.7 μL, 1.2 eq). The mixture was stirred at 80° C. for 12 hr. LC-MS showed desired compound was detected. The reaction mixture was filtered and concentrated under reduced pressure to give tert-butyl 4-(2-methoxy-2-oxo-ethyl)-3,5-dimethyl-piperazine-1-carboxylate (1.4 g, crude) as yellow oil.

To a solution of tert-butyl 4-(2-methoxy-2-oxo-ethyl)-3,5-dimethyl-piperazine-1-carboxylate (1.4 g, 4.7 mmol, 1 eq) in DMF (20 mL) was added Cs₂CO₃ (4.6 g, 14.1 mmol, 3 eq) and 4-(methylamino)-2-methylsulfanyl-pyrimidine-5-carbaldehyde (1.1 g, 5.7 mmol, 1.2 eq). The mixture was stirred at 120° C. for 12 hr. LC-MS showed desired compound was detected. The residue was diluted with H₂O (200 mL) and extracted with ethyl acetate (150 mL×3). The combined organic layers were washed with brine (100 mL×1), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 120 mL/min) to give tert-butyl 3,5-dimethyl-4-(8-methyl-2-methylsulfanyl-7-oxo-pyrido[2,3-d]pyrimidin-6-yl) piperazine-1-carboxylate (200 mg, 476.7 μmol, 10.1% yield) as a brown solid.

To a solution of tert-butyl 3,5-dimethyl-4-(8-methyl-2-methylsulfanyl-7-oxo-pyrido[2,3-d]pyrimidin-6-yl) piperazine-1-carboxylate (200 mg, 476.7 μmol, 1 eq) in DCM (2 mL) was added m-CPBA (174.2 mg, 858.1 μmol, 85% purity, 1.8 eq) at 0° C. The mixture was stirred at 25° C. for 12 hr. LC-MS showed desired compound was detected. The reaction mixture was partitioned between Na₂SO₃ (30 mL) and dichloromethane (30×3 mL). The organic phase was separated, washed with NaHCO₃ (20 mL×3), dried over Na₂SO₄, filtered and concentrated under reduced pressure to give tert-butyl 3,5-dimethyl-4-(8-methyl-2-methylsulfonyl-7-oxo-pyrido[2,3-d]pyrimidin-6-yl) piperazine-1-carboxylate (120 mg, crude) as a yellow solid.

To a solution of tert-butyl 3,5-dimethyl-4-(8-methyl-2-methylsulfonyl-7-oxo-pyrido[2,3-d]pyrimidin-6-yl) piperazine-1-carboxylate (120 mg, 265.7 μmol, 1 eq) in dioxane (2 mL) was added TFA (45.5 mg, 398.6 μmol, 29.5 μL, 1.5 eq) and 4-(4-methylpiperazin-1-yl)aniline (61.0 mg, 318.9 μmol, 1.2 eq). The mixture was stirred at 120° C. for 12 hr. LC-MS showed desired compound was detected. The reaction mixture was concentrated under reduced pressure to remove solvent. The residue was purified by prep-HPLC (FA condition; column: Phenomenex Luna C18 200×40 mm× 10 μm; mobile phase: [water (FA)-ACN];B %: 5%-40%, 8 min) to give tert-butyl 3,5-dimethyl-4-[8-methyl-2-[4-(4-methylpiperazin-1-yl)anilino]-7-oxo-pyrido[2,3-d]pyrimidin-6-yl]piperazine-1-carboxylate (26 mg, 46.2 μmol, 17.4% yield) as a brown solid.

A mixture of tert-butyl 3,5-dimethyl-4-[8-methyl-2-[4-(4-methylpiperazin-1-yl)anilino]-7-oxo-pyrido[2,3-d]pyrimidin-6-yl]piperazine-1-carboxylate (26 mg, 46.21 μmol, 1 eq) in HCl/EtOAc (1 mL, 4 M) was stirred at 25° C. for 1 hr. LC-MS showed desired compound was detected. The reaction mixture was filtered and concentrated under reduced pressure to give 6-(2, 6-dimethylpiperazin-1-yl)-8-methyl-2-[4-(4-methylpiperazin-1-yl)anilino]pyrido[2,3-d]pyrimidin-7-one (23 mg, crude, HCl) was obtained as a brown solid.

To a solution of 6-(2,6-dimethylpiperazin-1-yl)-8-methyl-2-[4-(4-methylpiperazin-1-yl)anilino]pyrido[2,3-d]pyrimidin-7-one (23 mg, 46.1 μmol, 1 eq, HCl) in DCM (1 mL) was added DIEA (17.8 mg, 138.3 μmol, 24.1 μL, 3 eq) and prop-2-enoyl chloride (4.2 mg, 46.1 μmol, 3.7 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition; column: Phenomenex Luna C18 200× 40 mm× 10 μm; mobile phase: [water (FA)-ACN];B %: 1%-25%, 8 min) to give 6-(2,6-dimethyl-4-prop-2-enoyl-piperazin-1-yl)-8-methyl-2-[4-(4-methylpiperazin-1-yl)anilino]pyrido[2,3-d]pyrimidin-7-one (11.4 mg, 22.1 μmol, 47.9% yield, 100% purity) as a yellow solid.

### Procedure for Preparation of Compound 024

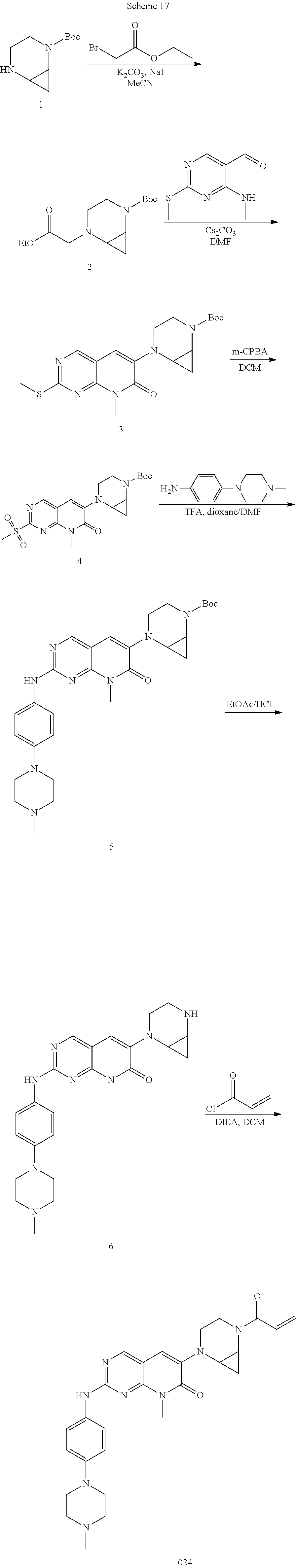

To a solution of tert-butyl 2,5-diazabicyclo[4.1.0]heptane-2-carboxylate (1.9 g, 9.6 mmol, 1 eq) in MeCN (30 mL) was added ethyl 2-bromoacetate (1.6 g, 9.6 mmol, 1.1 mL, 1 eq) and K₂CO₃ (4.0 g, 28.7 mmol, 3 eq). The reaction mixture was stirred at 60° C. for 1 hr. TLC plate (Ninhydrin) showed one new spot with lower polarity was formed. The reaction mixture was concentrated under reduced pressure to give a residue. The residue was purified by flash silica gel chromatography (40 g Silica Flash Column, Eluent of 0˜100% Ethyl acetate/Petroleum ether gradient @ 100 mL/min) to yield compound tert-butyl 5-(2-ethoxy-2-oxo-ethyl)-2,5-diazabicyclo[4.1.0]heptane-2-carboxylate (2.5 g, 8.8 mmol, 91.7% yield) obtained as a white solid.

To a solution of 4-(methylamino)-2-methylsulfanyl-pyrimidine-5-carbaldehyde (496.2 mg, 2.7 mmol, 1.1 eq) in DMF (5 mL) was added Cs₂CO₃ (2.4 g, 7.4 mmol, 3 eq) and tert-butyl 5-(2-ethoxy-2-oxo-ethyl)-2,5-diazabicyclo[4.1.0]heptane-2-carboxylate (700 mg, 2.5 mmol, 1 eq). The mixture was stirred at 80° C. for 12 hr (batch×3). The reaction was monitored by LCMS which showed the reactant was consumed completely and the desired mass peak was detected. The residue was diluted with H₂O (100 mL) and extracted acetate ethyl (100 ml×3). The combined organic layers were washed with brine (100 mL×2), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 100 mL/min) to yield compound tert-butyl 5-(8-methyl-2-methylsulfanyl-7-oxo-pyrido[2,3-d]pyrimidin-6-yl)-2,5-diazabicyclo[4.1.0]heptane-2-carboxylate (600 mg, 1.5 mmol, 60.4% yield) obtained as a yellow oil.

To a solution of tert-butyl 5-(8-methyl-2-methylsulfanyl-7-oxo-pyrido[2,3-d]pyrimidin-6-yl)-2,5-diazabicyclo[4.1.0]heptane-2-carboxylate (500 mg, 1.2 mmol, 1 eq) in DCM (10 mL) was added m-CPBA (377.4 mg, 1.9 mmol, 85% purity, 1.5 eq). The mixture was stirred at 0° C. for 2 hr (batch× 2). The reaction was monitored by LCMS which showed complete consumption of reactant and detection of the desired mass peak. The residue was diluted with Na₂SO₃ (10%, 10 mL) and extracted with DCM (20 mL× 3). The combined organic layers were washed with NaHCO₃ (20 mL× 3), dried over Na₂SO₄, filtered and concentrated under reduced pressure to give a residue. The residue was purified by prep-TLC (SiO₂, dichloromethane:methanol=20:1) to give compound tert-butyl 5-(8-methyl-2-methylsulfonyl-7-oxo-pyrido[2,3-d]pyrimidin-6-yl)-2,5-diazabicyclo[4.1.0]heptane-2-carboxylate (1 g, 2.30 mmol, 92.65% yield) obtained as a white solid.

To a solution of 4-(4-methylpiperazin-1-yl)aniline (112.0 mg, 585.5 μmol, 1.5 eq) in DMF (0.2 mL)/dioxane (2 mL) was added TFA (80.1 mg, 702.6 μmol, 52.0 μL, 1.8 eq) and tert-butyl 5-(8-methyl-2-methylsulfonyl-7-oxo-pyrido[2,3-d]pyrimidin-6-yl)-2,5-diazabicyclo[4.1.0]heptane-2-carboxylate (170 mg, 390.4 μmol, 1 eq). The mixture was stirred at 120° C. for 12 hr (batch×6). The reaction was monitored by LCMS which showed complete consumption of reactant and detection of the desired mass peak. The reaction mixture was concentrated under reduced pressure to give a residue. The residue was purified by prep-HPLC (FA condition; column: Phenomenex Luna C18 100×30 mm× 5 μm; mobile phase: [water (TFA)-ACN];B %: 10%-40%, 8 min) to yield compound tert-butyl 5-[8-methyl-2-[4-(4-methylpiperazin-1-yl)anilino]-7-oxo-pyrido[2,3-d]pyrimidin-6-yl]-2,5-diazabicyclo[4.1.0]heptane-2-carboxylate (350 mg) obtained as a yellow solid.

A solution of tert-butyl 5-[8-methyl-2-[4-(4-methylpiperazin-1-yl)anilino]-7-oxo-pyrido[2,3-d]pyrimidin-6-yl]-2,5-diazabicyclo[4.1.0]heptane-2-carboxylate (50 mg, 91.5 μmol, 1 eq) in HCl/EtOAc (4 mL, 4 M) was stirred at 25° C. for 10 min. The reaction was monitored by LCMS which showed complete consumption of reactant and detection of the desired product. The reaction mixture was concentrated under reduced pressure to give the crude product 6-(2,5-diazabicyclo[4.1.0]heptan-2-yl)-8-methyl-2-[4-(4-methylpiperazin-1-yl)anilino]pyrido[2,3-d]pyrimidin-7-one (60 mg, crude) obtained as yellow solid used into the next step without further purification.

To a solution of 6-(2,5-diazabicyclo[4.1.0]heptan-2-yl)-8-methyl-2-[4-(4-methylpiperazin-1-yl)anilino]pyrido[2,3-d]pyrimidin-7-one (30 mg, 62.1 μmol, 1 eq, HCl) in DCM (2 mL) was added DIEA (24.1 mg, 186.3 μmol, 32.4 μL, 3 eq) and prop-2-enoyl chloride (5.6 mg, 62.1 μmol, 5.1 μL, 1 eq) at 0° C. The mixture was stirred at 25° C. for1 hr. The reaction was monitored by LCMS which showed complete consumption of reactant and detection of the desired mass peak. The reaction mixture was concentrated under reduced pressure to remove solvent. The residue was diluted with dimethyl formamide (3 mL). The residue was purified by prep-HPLC (FA condition; column: Phenomenex Luna C18 100×30 mm× 5 μm; mobile phase: [water (TFA)-ACN];B %: 1%-30%, 8 min). The residue was purified by prep-HPLC (neutral condition; column: Waters Xbridge Prep OBD C18 150×40 mm×10 μm; mobile phase: [water (NH₄HCO₃)-ACN];B %: 20%-50%, 8 min) to yield compound 8-methyl-2-[4-(4-methylpiperazin-1-yl)anilino]-6-(5-prop-2-enoyl-2,5-diazabicyclo[4.1.0]heptan-2-yl)pyrido[2,3-d]pyrimidin-7-one (5 mg, 9.0 μmol, 14.5% yield, 90.488% purity) obtained as a white solid. Analytic data 1H NMR and QC were consistent with the desired product.

### Procedure for Preparation of Compound 025

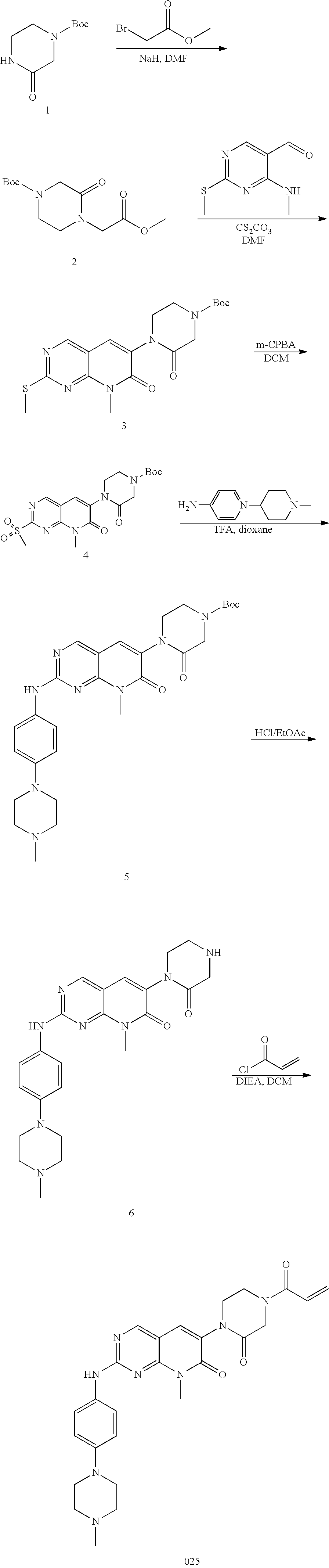

To a solution of tert-butyl 3-oxopiperazine-1-carboxylate (2 g, 10.0 mmol, 1 eq) in DMF (30 mL) was added NaH (599.2 mg, 15.0 mmol, 60% purity, 1.5 eq) at 0° C. The mixture was stirred at 65° C. for 1 hr. After the mixture was cooled to room temperature, methyl 2-bromoacetate (1.8 g, 12.0 mmol, 1.1 mL, 1.2 eq) was added to the mixture. The mixture was stirred at 65° C. for 12 hr. TLC indicated Reactant 1 was consumed completely and one new spot formed. The residue was diluted with (H₂O 200 mL) and extracted with ethyl acetate (150 mL× 3). The combined organic layers were washed with brine (100 mL× 1), dried over Na₂SO₄, filtered and concentrated under reduced pressure to give a residue. The residue was purified by flash silica gel chromatography (20 g Silica Flash Column, Eluent of 0˜20% Ethyl acetate/Petroleum ether gradient @ 120 mL/min) to give tert-butyl 4-(2-methoxy-2-oxo-ethyl)-3-oxo-piperazine-1-carboxylate (1.7 g, 6.2 mmol, 62.5% yield) as yellow oil.

To a solution of tert-butyl 4-(2-methoxy-2-oxo-ethyl)-3-oxo-piperazine-1-carboxylate (1 g, 3.7 mmol, 1 eq) in DMF (10 mL) was added Cs₂CO₃ (3.6 g, 11.0 mmol, 3 eq) and 4-(methylamino)-2-methylsulfanyl-pyrimidine-5-carbaldehyde (740.2 mg, 4.1 mmol, 1.1 eq). The mixture was stirred at 40° C. for 12 hr. LC-MS showed desired compound was detected. The residue was diluted with H₂O (200 mL) and extracted with ethyl acetate (150 mL× 3). The combined organic layers were washed with brine (100 mL× 2), dried over Na₂SO₄, filtered and concentrated under reduced pressure to give a residue. The residue was purified by flash silica gel chromatography (40 g Silica Flash Column, Eluent of 0˜20% Ethyl acetate/Petroleum ether gradient @ 100 mL/min) to give tert-butyl 4-(8-methyl-2-methylsulfanyl-7-oxo-pyrido[2,3-d]pyrimidin-6-yl)-3-oxo-piperazine-1-carboxylate (2.5 g, 6.2 mmol, 56.0% yield) as a yellow solid.

To a solution of tert-butyl 4-(8-methyl-2-methylsulfanyl-7-oxo-pyrido[2,3-d]pyrimidin-6-yl)-3-oxo-piperazine-1-carboxylate (1.1 g, 2.7 mmol, 1 eq) in DCM (15 mL) was added m-CPBA (826.2 mg, 4.1 mmol, 85% purity, 1.5 eq) at 0° C. The mixture was stirred at 25° C. for 12 hr. LC-MS showed desired compound was detected. The reaction mixture was partitioned between Na₂SO₃ (250 mL) and dichloromethane (250×3 mL). The organic phase was separated, washed with NaHCO₃ (50 mL×3), dried over Na₂SO₄, filtered and concentrated under reduced pressure. The residue was purified by flash silica gel chromatography (12 g Silica Flash Column, Eluent of 0˜100% Ethyl acetate/Petroleum ether gradient @ 120 mL/min) to give tert-butyl 4-(8-methyl-2-methylsulfonyl-7-oxo-pyrido[2,3-d]pyrimidin-6-yl)-3-oxo-piperazine-1-carboxylate (520 mg, 1.2 mmol, 43.8% yield) as a yellow solid.

To a solution of tert-butyl 4-(8-methyl-2-methylsulfonyl-7-oxo-pyrido[2,3-d]pyrimidin-6-yl)-3-oxo-piperazine-1-carboxylate (460 mg, 525.8 μmol, 1 eq) in dioxane (2 mL) was added TFA (178.8 mg, 788.6 μmol, 58.4 μL, 1.5 eq) and 4-(4-methylpiperazin-1-yl)aniline (241.4 mg, 630.9 μmol, 1.2 eq). The mixture was stirred at 120° C. for 12 hr. LC-MS showed desired compound was detected. The reaction mixture was concentrated under reduced pressure to remove solvent. The residue was purified by prep-HPLC (FA condition; column: Phenomenex Luna C18 100×30 mm×5 μm; mobile phase: [water (FA)-ACN];B %: 5%-30%, 8 min) to give tert-butyl 4-[8-methyl-2-[4-(4-methylpiperazin-1-yl)anilino]-7-oxo-pyrido[2,3-d]pyrimidin-6-yl]-3-oxo-piperazine-1-carboxylate (340 mg, 619.7 μmol, 58.9% yield) as a yellow solid.

A mixture of tert-butyl 4-[8-methyl-2-[4-(4-methylpiperazin-1-yl)anilino]-7-oxo-pyrido[2,3-d]pyrimidin-6-yl]-3-oxo-piperazine-1-carboxylate (340 mg, 619.7 μmol, 1 eq) in HCl/EtOAc (5 mL, 4 M) was stirred at 25° C. for 2 hr. LC-MS showed desired compound was detected. The reaction mixture was filtered and concentrated under reduced pressure to give 8-methyl-2-[4-(4-methylpiperazin-1-yl)anilino]-6-(2-oxopiperazin-1-yl)pyrido[2,3-d]pyrimidin-7-one (330 mg, crude) as a yellow solid.

To a solution of 8-methyl-2-[4-(4-methylpiperazin-1-yl)anilino]-6-(2-oxopiperazin-1-yl)pyrido[2,3-d]pyrimidin-7-one (150 mg, 334.4 μmol, 1 eq) in DCM (2 mL) was added DIEA (129.7 mg, 1.00 mmol, 174.8 μL, 3 eq) and prop-2-enoyl chloride (30.3 mg, 334.4 μmol, 27.3 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition; column: Phenomenex C18 75× 30 mm× 3 μm; mobile phase: [water (TFA)-ACN];B %: 1%-20%, 8 min) to give 8-methyl-2-[4-(4-methylpiperazin-1-yl)anilino]-6-(2-oxo-4-prop-2-enoyl-piperazin-1-yl)pyrido[2,3-d]pyrimidin-7-one (20 mg, 39.8 μmol, 11.9% yield, 99.1% purity) as a yellow solid.

### Procedure for Preparation of Compound 027

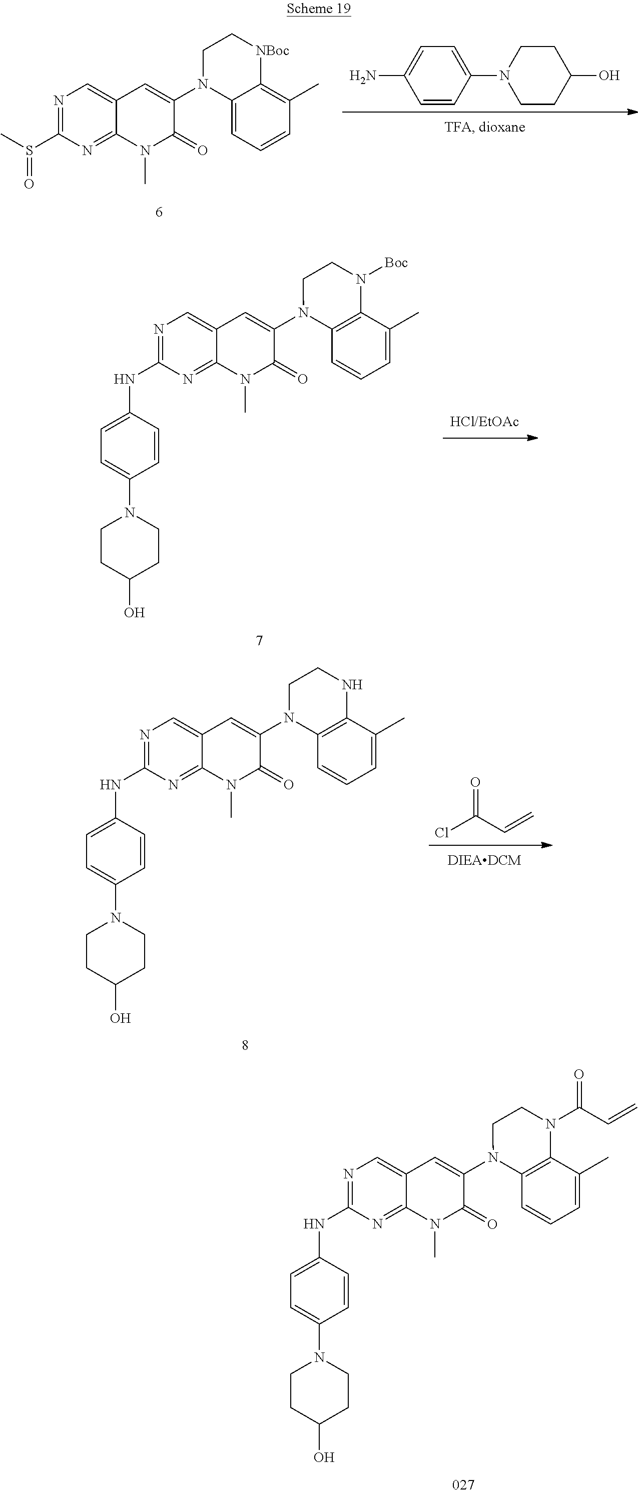

To a solution of tert-butyl 8-methyl-4-(8-methyl-2-methylsulfinyl-7-oxo-pyrido[2,3-d]pyrimidin-6-yl)-2,3-dihydroquinoxaline-1-carboxylate (150.0 mg, 319.5 μmol, 1 eq) in dioxane (2 mL) was added TFA (43.71 mg, 383.3 μmol, 28.4 μL, 1.2 eq) and 1-(4-aminophenyl) piperidin-4-ol (73.7 mg, 383.3 μmol, 1.2 eq). The mixture was stirred at 100° C. for 12 hr. LC-MS showed desired compound was detected. The mixture was concentrated to get a residue. The residue was purified by prep-HPLC (TFA condition; column: Phenomenex C18 75×30 mm×3 μm;mobile phase: [water (TFA)-ACN];B %: 45%-75%, 8 min).to give tert-butyl 4-[2-[4-(4-hydroxy-1-piperidyl)anilino]-8-methyl-7-oxo-pyrido[2,3-d]pyrimidin-6-yl]-8-methyl-2,3-dihydroquinoxaline-1-carboxylate (110.0 mg, 184.0 μmol, 57.6% yield) as a brown solid.

To a solution of tert-butyl 4-[2-[4-(4-hydroxy-1-piperidyl)anilino]-8-methyl-7-oxo-pyrido[2,3-d]pyrimidin-6-yl]-8-methyl-2,3-dihydroquinoxaline-1-carboxylate (110.0 mg, 184.0 μmol, 1 eq) in EtOAc (2 mL) was added HCl/EtOAc (4 M, 1 mL), the mixture was stirred at 25° C. for 1 hr. LC-MS showed desired compound was detected. The mixture concentrated to give 2-[4-(4-hydroxy-1-piperidyl)anilino]-8-methyl-6-(5-methyl-3,4-dihydro-2H-quinoxalin-1-yl)pyrido[2,3-d]pyrimidin-7-one (120 mg, crude, HCl) as a yellow solid used into the next step without further purification.

To a solution of 2-[4-(4-hydroxy-1-piperidyl)anilino]-8-methyl-6-(5-methyl-3,4-dihydro-2H-quinoxalin-1-yl)pyrido[2,3-d]pyrimidin-7-one (50.0 mg, 100.5 μmol, 1 eq) in DCM (1 mL) was added TEA (30.5 mg, 301.5 μmol, 42.0 μL, 3 eq) and prop-2-enoyl chloride (9.1 mg, 100.5 μmol, 8.2 μL, 1 eq). The mixture was stirred at 25° C. for 1 hr. LC-MS showed desired compound was detected. The mixture was concentrated to get a residue. The residue was purified by prep-HPLC (FA condition; column: Phenomenex luna C18 100×40 mm×5 μm; mobile phase: [water (FA)-ACN];B %: 10%-40%, 8 min) to give 2-[4-(4-hydroxy-1-piperidyl)anilino]-8-methyl-6-(5-methyl-4-prop-2-enoyl-2,3-dihydroquinoxalin-1-yl)pyrido[2,3-d]pyrimidin-7-one (20.2 mg, 33.20 μmol, 33.04% yield, 98.24% purity, FA) as a yellow solid.

### Procedure for Preparation of Compound 033

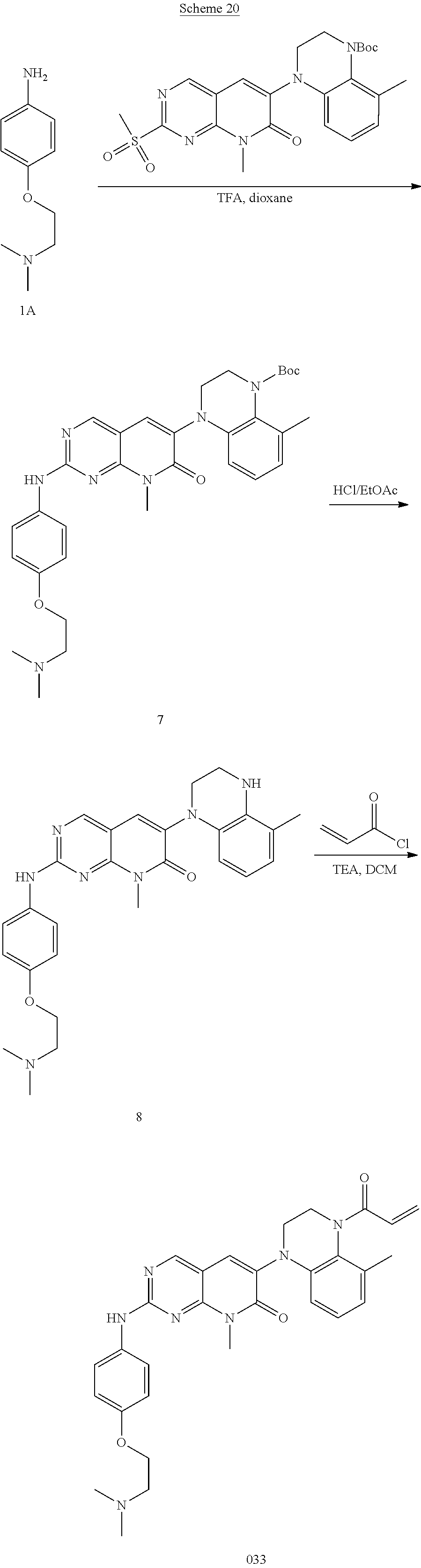

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (300 mg, 617.8 μmol, 1 eq),4-[2-(dimethylamino)ethoxy]aniline (222.8 mg, 1235.8 μmol, 2 eq) in dioxane (3 mL) was added TFA (141.0 mg, 1235.8 μmol, 91.6 μL, 2 eq). The mixture was stirred at 120° C. for 12 hr. LC-MS showed desired compound was detected. The reaction mixture filtered and concentrated under reduced pressure to give a residue. The residue was purified by prep-HPLC (TFA condition column: Phenomenex luna C18 100×40 mm×5 μm; mobile phase: [water (TFA)-ACN];B %: 20%-50%, 8 min) to give tert-butyl 4-[2-[4-[2-(dimethylamino)ethoxy]anilino]-8-methyl-7-oxo-pyrido[2,3-d]pyrimidin-6-yl]-8-methyl-2,3-dihydroquinoxaline-1-carboxylate (155 mg, 264.6 μmol, 85.7% yield) as a yellow solid.

To a solution of tert-butyl 4-[2-[4-[2-(dimethylamino)ethoxy]anilino]-8-methyl-7-oxo-pyrido[2,3-d]pyrimidin-6-yl]-8-methyl-2,3-dihydroquinoxaline-1-carboxylate (138 mg, 235.6 μmol, 1 eq) in HCl/EtOAc (2 mL, 4M) at 25° C. The mixture was stirred at 25° C. for 1 hr. LC-MS showed desired compound was detected. The reaction mixture filtered and concentrated under reduced pressure to give 2-[4-[2-(dimethylamino)ethoxy]anilino]-8-methyl-6-(5-methyl-3,4-dihydro-2H-quinoxalin-1-yl)pyrido[2,3-d]pyrimidin-7-one (180 mg, crude, HCl) as a yellow solid.

To a solution of 2-[4-[2-(dimethylamino)ethoxy]anilino]-8-methyl-6-(5-methyl-3,4-dihydro-2H-quinoxalin-1-yl)pyrido[2,3-d]pyrimidin-7-one (150 mg, 287.3 μmol, 1 eq, HCl) in DCM (3 mL) was added TEA (87.2 mg, 862.0 μmol, 120.0 μL, 3 eq) and prop-2-enoyl chloride (26.0 mg, 287.3 μmol, 23.4 μL, 1 eq) at 0° C. The mixture was stirred at 0° C. for 1 hr. LC-MS showed desired compound was detected. The reaction mixture filtered and concentrated under reduced pressure to give a residue. The residue was purified by prep-HPLC (TFA condition column: Phenomenex luna C18 100×40 mm× 5 μm; mobile phase: [water (TFA)-ACN];B %: 5%-40%, 8 min) to give 2-[4-[2-(dimethylamino)ethoxy]anilino]-8-methyl-6-(5-methyl-4-prop-2-enoyl-2,3-dihydroquinoxalin-1-yl)pyrido[2,3-d]pyrimidin-7-one (47.3 mg, 87.6 μmol, 30.5% yield, 100% purity, TFA) as a yellow solid.

### Procedure for Preparation of Compound 034

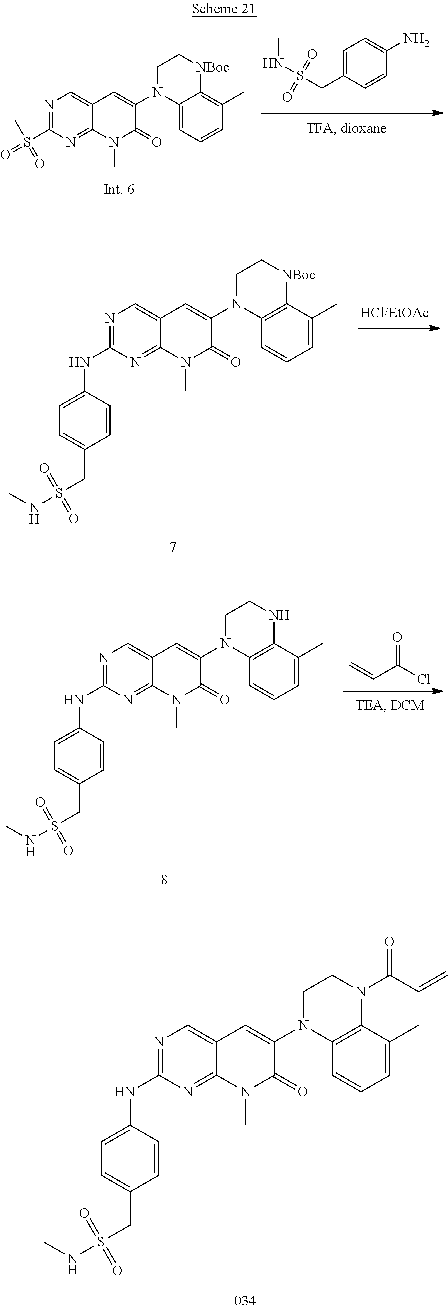

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (200 mg, 411.9 μmol, 1 eq) in dioxane (2 mL) was added 1-(4-aminophenyl)-N-methyl-methanesulfonamide (123.7 mg, 617.9 μmol, 1.5 eq) and TFA (70.5 mg, 617.9 μmol, 45.9 μL, 1.5 eq). The mixture was stirred at 110° C. for 12 hr. LC-MS showed desired compound was detected. The reaction mixture was concentrated under reduced pressure to remove solvent. The residue was purified by prep-HPLC (TFA condition column: Phenomenex luna C18 100×40 mm× 5 μm; mobile phase: [water (TFA)-ACN];B %: 40%-70%, 8 min) to give tert-butyl 8-methyl-4-[8-methyl-2-[4-(methylsulfamoylmethyl)anilino]-7-oxo-pyrido[2,3-d]pyrimidin-6-yl]-2,3-dihydroquinoxaline-1-carboxylate (190 mg, 313.7 μmol, 76.2% yield) as a yellow solid.

To a solution of tert-butyl 8-methyl-4-[8-methyl-2-[4-(methylsulfamoylmethyl)anilino]-7-oxo-pyrido[2,3-d]pyrimidin-6-yl]-2,3-dihydroquinoxaline-1-carboxylate (90 mg, 148.6 μmol, 1 eq) in DCM (1 mL) was added TFA (338.9 mg, 3.0 mmol, 220.0 μL, 20 eq). The mixture was stirred at 25° C. for 1 hr. LC-MS showed desired compound was detected. The reaction mixture was concentrated under reduced pressure to give N-methyl-1-[4-[[8-methyl-6-(5-methyl-3,4-dihydro-2H-quinoxalin-1-yl)-7-oxo-pyrido[2,3-d]pyrimidin-2-yl]amino]phenyl]methanesulfonamide (90 mg, crude, TFA) as a yellow oil.

To a solution of N-methyl-1-[4-[[8-methyl-6-(5-methyl-3,4-dihydro-2H-quinoxalin-1-yl)-7-oxo-pyrido[2,3-d]pyrimidin-2-yl]amino]phenyl]methanesulfonamide (40 mg, 79.1 μmol, 1 eq) in DCM (1 mL) was added TEA (24.0 mg, 237.4 μmol, 33.0 μL, 3 eq) and prop-2-enoyl chloride (7.2 mg, 79.1 μmol, 6.5 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition column: Phenomenex Luna C18 200×40 mm× 10 μm; mobile phase: [water (FA)-ACN];B %: 30%-65%, 8 min) to give N-methyl-1-[4-[[8-methyl-6-(5-methyl-4-prop-2-enoyl-2,3-dihydroquinoxalin-1-yl)-7-oxo-pyrido[2,3-d]pyrimidin-2-yl]amino]phenyl]methanesulfonamide (20.4 mg, 36.5 μmol, 46.1% yield, 100% purity) as a yellow solid.

### Procedure for Preparation of Compound 061

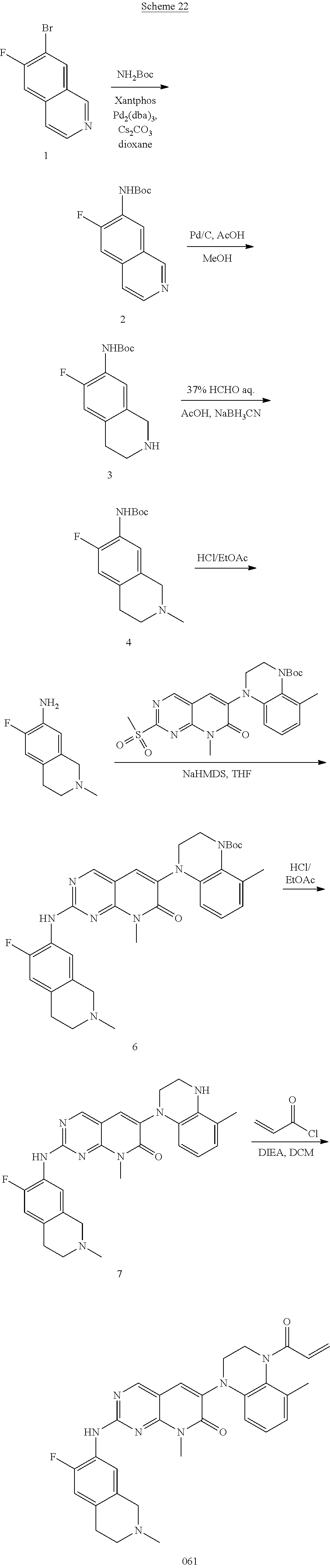

To a solution of 7-bromo-6-fluoro-isoquinoline (926.0 mg, 4.1 mmol, 1 eq) in dioxane (10 mL) was added tert-butyl carbamate (959.8 mg, 8.2 mmol, 2 eq), Cs₂CO₃ (4.0 g, 12.3 mmol, 3 eq) and XPhos Pd G3 (693.5 mg, 819.3 μmol, 0.2 eq) under N₂ atmosphere. The mixture was stirred at 100° C. for 12 hr. LC-MS showed desired compound was detected. The reaction mixture was diluted with H₂O (10 mL) and extracted with ethyl acetate (10 mL× 3). The combined organic layers were washed with brine (15 mL), dried over Na₂SO₄, filtered and concentrated under reduced pressure to give a residue. The residue was purified by flash silica gel chromatography (12 g Silica Flash Column, Eluent of 0˜100% Ethyl acetate/Petroleum ether gradient @ 80 mL/min). to give compound tert-butyl N-(6-fluoro-7-isoquinolyl) carbamate (926 mg, 3.5 mmol, 86.2% yield) as a white solid.

To a mixture of Pd/C (300 mg, 10% purity) in MeOH (10 mL) was added tert-butyl N-(6-fluoro-7-isoquinolyl) carbamate (926 mg, 3.5 mmol, 1 eq) and AcOH(636.1 mg, 10.6 mmol, 606.4 μL, 3 eq) under N₂ atmosphere. The suspension was degassed and purged with H₂ for 3 times. The mixture was stirred under H₂ (50 Psi) at 40° C. for 12 hr. LC-MS showed desired compound was detected. The reaction was filtered to remove the undissolved substance to give tert-butyl N-(6-fluoro-1,2,3,4-tetrahydroisoquinolin-7-yl) carbamate (920 mg, crude) as a yellow oil.

To a solution of tert-butyl N-(6-fluoro-1,2,3,4-tetrahydroisoquinolin-7-yl) carbamate (800 mg, 3.0 mmol, 1 eq) and formaldehyde (1.2 g, 15.0 mmol, 1.1 mL, 5 eq) (37% in water) in MeOH (10 mL) was added AcOH to adjust the pH to 4-5, then AcOH(180.4 mg, 3.0 mmol, 171.9 μL, 1 eq) and NaBH₃CN (566.3 mg, 9.0 mmol, 3 eq) was added to the mixture, the mixture was stirred at 25° C. for 12 hr. LC-MS showed desired compound was detected. The reaction was concentrated under reduce pressure to give a residue. The residue was purified by prep-HPLC (HCl condition; column: Phenomenex Luna C18 75×30 mm×3 μm; mobile phase: [H₂O (0.04% HCl)-ACN]; gradient: 5%-35% B over 8.0 min) to give compound tert-butyl N-(6-fluoro-2-methyl-3,4-dihydro-1H-isoquinolin-7-yl) carbamate (250 mg, 891.8 μmol, 29.7% yield) as a white solid.

A solution of tert-butyl N-(6-fluoro-2-methyl-3,4-dihydro-1H-isoquinolin-7-yl) carbamate (200 mg, 713.4 μmol, 1 eq) in HCl/EtOAc (2 mL, 4M) was stirred at 25° C. for 1 hr. LC-MS showed desired compound was detected. The reaction was concentrated under reduce pressure to give compound 6-fluoro-2-methyl-3,4-dihydro-1H-isoquinolin-7-amine (147 mg, crude) as a white solid.

To a solution of 6-fluoro-2-methyl-3,4-dihydro-1H-isoquinolin-7-amine (37 mg, 205.3 μmol, 1.5 eq) in THF (2 mL) was added NaHMDS(1 M, 273.7 μL, 2 eq) at −78° C. under N₂ atmosphere. The mixture was stirred at −78° C. for 5 min. Tert-butyl 8-methyl-4-(8-methyl-2-methylsulfonyl-7-oxo-pyrido[2,3-d]pyrimidin-6-yl)-2,3-dihydroquinoxaline-1-carboxylate (66.5 mg, 136.9 μmol, 1 eq) in THF (2 mL) was added to the mixture, the mixture was stirred at −78° C. for 55 min. LC-MS showed desired compound was detected. The reaction mixture was quenched by addition sat·NH₄CI (5 mL) at 25° C., and extracted with acetate ethyl (8 mL× 3). The combined organic layers were dried over Na₂SO₄, filtered and concentrated under reduced pressure to give a residue. The residue was purified by prep-HPLC (TFA condition; column: Phenomenex luna C18 100×40 mm× 5 μm; mobile phase: [H₂O (0.1% TFA)-ACN]; gradient: 25%-55% B over 8.0 min). to give compound tert-butyl 4-[2-[(6-fluoro-2-methyl-3,4-dihydro-1H-isoquinolin-7-yl)amino]-8-methyl-7-oxo-pyrido[2,3-d]pyrimidin-6-yl]-8-methyl-2,3-dihydroquinoxaline-1-carboxylate (14 mg, 23.9 μmol, 17.5% yield, 100% purity) as a white solid.

A solution of tert-butyl 4-[2-[(6-fluoro-2-methyl-3,4-dihydro-1H-isoquinolin-7-yl)amino]-8-methyl-7-oxo-pyrido[2,3-d]pyrimidin-6-yl]-8-methyl-2,3-dihydroquinoxaline-1-carboxylate (14 mg, 23.9 μmol, 1 eq) in HCl/EtOAc (1 mL, 4M) was stirred at 25° C. for 1 hr. LC-MS showed desired compound was detected. The reaction was concentrated under reduce pressure to give compound 2-[(6-fluoro-2-methyl-3,4-dihydro-1H-isoquinolin-7-yl)amino]-8-methyl-6-(5-methyl-3,4-dihydro-2H-quinoxalin-1-yl)pyrido[2,3-d]pyrimidin-7-one (9 mg, crude, HCl) as a white solid.

To a solution of 2-[(6-fluoro-2-methyl-3,4-dihydro-1H-isoquinolin-7-yl)amino]-8-methyl-6-(5-methyl-3,4-dihydro-2H-quinoxalin-1-yl)pyrido[2,3-d]pyrimidin-7-one (9 mg, 17.2 μmol, 1 eq, HCl) in DCM (1 mL) was added DIEA to adjust the pH to 7-8, DIEA (6.7 mg, 51.7 μmol, 9.0 μL, 3 eq) and prop-2-enoyl chloride (1.7 mg, 18.9 μmol, 1.5 μL, 1.1 eq) was added to the mixture at 0° C., the mixture was stirred at 0° C. for 1 hr. LC-MS showed desired compound was detected. The residue was purified by prep-HPLC (FA condition; column: Phenomenex Luna C18 100× 30 mm× 5 μm; mobile phase: [H₂O (0.2% FA)-ACN]; gradient: 15%-50% B over 8.0 min). to give compound 2-[(6-fluoro-2-methyl-3,4-dihydro-1H-isoquinolin-7-yl)amino]-8-methyl-6-(5-methyl-4-prop-2-enoyl-2,3-dihydroquinoxalin-1-yl)pyrido[2,3-d]pyrimidin-7-one (2.6 mg, 4.4 μmol, 25.5% yield, 100% purity, FA) as a white solid.

### Procedure for Preparation of Compound 037

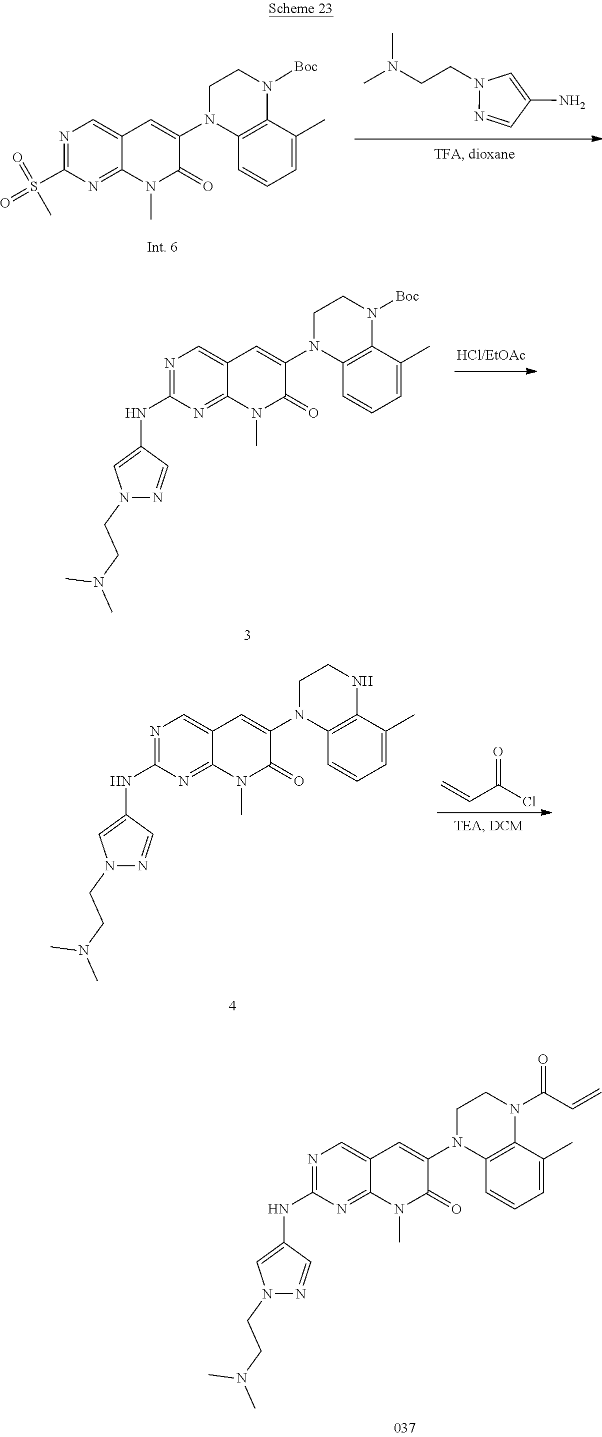

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (100 mg, 205.9 μmol, 1 eq) in dioxane (1 mL) was added 1-[2-(dimethylamino)ethyl]pyrazol-4-amine (47.6 mg, 308.9 μmol, 1.5 eq) and TFA (35.2 mg, 308.9 μmol, 22.8 μL, 1.5 eq). The resulting mixture was stirred at 100° C. for 12 hr (batch× 2). LC-MS showed desired compound was detected. The reaction mixture was concentrated under reduced pressure to give a residue. The residue was purified by prep-HPLC (TFA condition) column: Phenomenex luna C18 100×40 mm× 5 μm; mobile phase: [water (TFA)-ACN];B %: 25%-55%, 8 min to give tert-butyl 4-[2-[[1-[2-(dimethylamino)ethyl]pyrazol-4-yl]amino]-8-methyl-7-oxo-pyrido[2,3-d]pyrimidin-6-yl]-8-methyl-2,3-dihydroquinoxaline-1-carboxylate (130 mg, 232.2 μmol, 37.6% yield) as a yellow solid.

A mixture of tert-butyl 4-[2-[[1-[2-(dimethylamino)ethyl]pyrazol-4-yl]amino]-8-methyl-7-oxo-pyrido[2,3-d]pyrimidin-6-yl]-8-methyl-2,3-dihydroquinoxaline-1-carboxylate (130 mg, 232.2 μmol, 1 eq) in HCl/EtOAc (2 mL 4 M) was stirred at 20° C. for 1 hr. LC-MS showed desired compound was detected. The reaction mixture was concentrated under reduced pressure to give 2-[[1-[2-(dimethylamino)ethyl]pyrazol-4-yl]amino]-8-methyl-6-(5-methyl-3,4-dihydro-2H-quinoxalin-1-yl)pyrido[2,3-d]pyrimidin-7-one (152.8 mg, crude) as a yellow solid.

To a solution of 2-[[1-[2-(dimethylamino)ethyl]pyrazol-4-yl]amino]-8-methyl-6-(5-methyl-3,4-dihydro-2H-quinoxalin-1-yl)pyrido[2,3-d]pyrimidin-7-one (140 mg, 304.6 μmol, 1 eq) in DCM (7 mL) was added dropwise TEA (92.4 mg, 913.9 μmol, 127.2 μL, 3 eq) and prop-2-enoyl chloride (27.5 mg, 304.6 μmol, 24.8 μL, 1 eq) in DCM (0.1 mL) at 0° C. The resulting mixture was stirred at 0° C. for 30 min. LC-MS showed desired compound was detected. The reaction mixture was concentrated under N₂ to remove solvent. The residue was purified by prep-HPLC (neutral condition column: Waters Xbridge BEH C18 100×30 mm× 10 μm; mobile phase: [water (NH₄HCO₃)-ACN];B %: 25%-55%, 8 min) to give 2-[[1-[2-(dimethylamino)ethyl]pyrazol-4-yl]amino]-8-methyl-6-(5-methyl-4-prop-2-enoyl-2,3-dihydroquinoxalin-1-yl)pyrido[2,3-d]pyrimidin-7-one (14.9 mg, 29 μmol, 9.5% yield, 100% purity) as a yellow solid.

### Procedure for Preparation of Compound 048

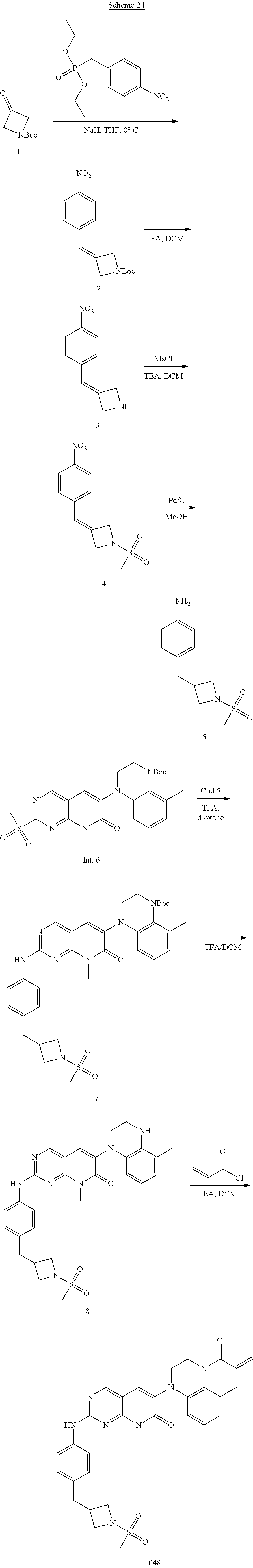

To a solution of NaH (175.7 mg, 4.4 mmol, 60% purity, 1.2 eq) in THF (10 mL) was added dropwise 1-(diethoxyphosphorylmethyl)-4-nitro-benzene (1 g, 3.7 mmol, 1 eq) in THF (5 mL) at 0° C. under N₂. The mixture was stirred at 0° C. for 30 min. After 30 min, a solution of tert-butyl 3-oxoazetidine-1-carboxylate (626.6 mg, 3.7 mmol, 1 eq) in THF (5 mL) was added dropwise at 0° C. The mixture was allowed to slowly warm to 25° C. The mixture was stirred at 25° C. for 2 hr. LC-MS showed desired compound was detected. The residue was purified by flash silica gel chromatography (20 g Silica Flash Column, Eluent of 0˜20% Ethyl acetate/Petroleum ether gradient @ 100 mL/min) to give tert-butyl 3-[(4-nitrophenyl)methylene]azetidine-1-carboxylate (450 mg, 1.6 mmol, 42.4% yield) as a brown solid.

To a solution of tert-butyl 3-[(4-nitrophenyl)methylene]azetidine-1-carboxylate (200 mg, 688.9 μmol, 1 eq) in DCM (3 mL) was added TFA (392.7 mg, 3.4 mmol, 255.0 μL, 5 eq). The mixture was stirred at 25° C. for 12 hr. LC-MS showed desired compound was detected. The reaction mixture was concentrated under reduced pressure to remove solvent to give 3-[(4-nitrophenyl)methylene]azetidine (300 mg, crude, TFA) as brown oil.

To a solution of 3-[(4-nitrophenyl)methylene]azetidine (600 mg, 3.15 mmol, 1 eq) in DCM (10 mL) was added TEA (1.3 g, 12.6 mmol, 1.7 mL, 4 eq) and MsCl (433.6 mg, 3.8 mmol, 293.0 μL, 1.2 eq) at 0° C. The mixture was stirred at 25° C. for 2 hr. LC-MS showed desired compound was detected. The reaction mixture was partitioned between NaHCO₃ (20 mL) and dichloromethane (30 mL× 3). The organic phase was separated, washed with brine (10 mL×1), dried over Na₂SO₄, filtered and concentrated under reduced pressure to give a residue. The residue was purified by flash silica gel chromatography (4 g Silica Flash Column, Eluent of 0˜20% Ethyl acetate/Petroleum ether gradient @ 120 mL/min) to give 1-methylsulfonyl-3-[(4-nitrophenyl)methylene]azetidine (370 mg, 1.4 mmol, 43.7% yield) as a white solid.

To a solution of Pd/C (50 mg, 10% purity) in MeOH (3 mL) was added 1-methylsulfonyl-3-[(4-nitrophenyl)methylene]azetidine (100 mg, 372.7 μmol, 1 eq) under N₂ atmosphere. The suspension was degassed and purged with H₂ for 3 times. The mixture was stirred under H₂ (50 psi) at 25° C. for 12 hr. LC-MS showed desired compound was detected. The reaction mixture was filtered and concentrated under reduced pressure to give 4-[(1-methylsulfonylazetidin-3-yl)methyl]aniline (96 mg, crude) as a yellow solid.

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (200 mg, 411.9 μmol, 1 eq) in dioxane (2 mL) was added TFA (70.5 mg, 617.9 μmol, 45.9 μL, 1.5 eq) and 4-[(1-methylsulfonylazetidin-3-yl)methyl]aniline (217.8 mg, 906.2 μmol, 2.2 eq). The mixture was stirred at 80° C. for 12 hr. LC-MS showed desired compound was detected. The reaction mixture was concentrated under reduced pressure to remove solvent. The residue was purified by prep-HPLC (FA condition; column: Phenomenex Luna C18 200×40 mm× 10 μm; mobile phase: [water (FA)-ACN];B %: 50%-80%, 8 min) to give tert-butyl 8-methyl-4-[8-methyl-2-[4-[(1-methylsulfonylazetidin-3-yl)methyl]anilino]-7-oxo-pyrido[2,3-d]pyrimidin-6-yl]-2,3-dihydroquinoxaline-1-carboxylate (160 mg, 247.9 μmol, 30.1% yield) as a yellow solid.

To a solution of tert-butyl 8-methyl-4-[8-methyl-2-[4-[(1-methylsulfonylazetidin-3-yl)methyl]anilino]-7-oxo-pyrido[2,3-d]pyrimidin-6-yl]-2,3-dihydroquinoxaline-1-carboxylate (80 mg, 123.9 μmol, 1 eq) in DCM (2 mL) was added TFA (70.6 mg, 619.4 μmol, 46.0 μL, 5 eq). The mixture was stirred at 20° C. for 20 hr. LC-MS showed desired compound was detected. The reaction mixture was filtered and concentrated under reduced pressure to give 8-methyl-6-(5-methyl-3,4-dihydro-2H-quinoxalin-1-yl)-2-[4-[(1-methylsulfonylazetidin-3-yl)methyl]anilino]pyrido[2,3-d]pyrimidin-7-one (80 mg, crude, TFA) as brown oil.

To a solution of 8-methyl-6-(5-methyl-3,4-dihydro-2H-quinoxalin-1-yl)-2-[4-[(1-methylsulfonylazetidin-3-yl)methyl]anilino]pyrido[2,3-d]pyrimidin-7-one (60 mg, 109.96 μmol, 1 eq) in DCM (2 mL) was added TEA (33.4 mg, 329.9 μmol, 45.9 μL, 3 eq) and prop-2-enoyl chloride (10.0 mg, 110.0 μmol, 8.9 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition; column: Phenomenex Luna C18 75×30 mm×3 μm; mobile phase: [H₂O (0.1% TFA)-ACN]; gradient: 40%-70% B over 8.0 min) to give 8-methyl-6-(5-methyl-4-prop-2-enoyl-2,3-dihydroquinoxalin-1-yl)-2-[4-[(1-methylsulfonylazetidin-3-yl)methyl]anilino]pyrido[2,3-d]pyrimidin-7-one (34.0 mg, 56.6 μmol, 51.5% yield, 100% purity) as a yellow solid.

### Procedure for Preparation of Compound 039

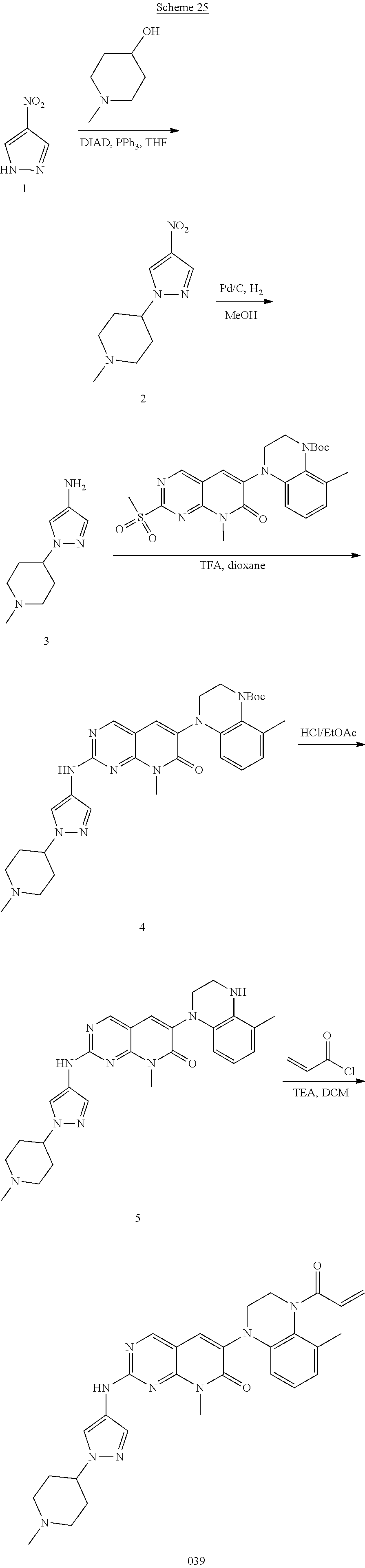

At 0° C., DIAD (5.3 g, 26.5 mmol, 5.1 mL, 1.5 eq) was added slowly to a mixture of 1-methylpiperidin-4-ol (2.4 g, 21.2 mmol, 2.4 mL, 1.2 eq), 4-nitro-1H-pyrazole (2 g, 17.6 mmol, 1 eq), PPh₃ (6.9 g, 26.5 mmol, 1.5 eq) in THF (20 mL), and the mixture was degassed and purged with N2 for 3 times, then the mixture was stirred at 20° C. for 16 hr under N₂ atmosphere. LC-MS showed desired compound was detected. The reaction mixture was quenched by addition ethyl acetate (30 mL) and HCl (30 mL), and the aqueous layer was treated with saturated aqueous K₂CO₃ solution to adjust pH=9, then extracted with ethyl acetate (30 mL× 3). The combined organic layers were washed with H₂O (30 mL×3), dried over Na₂SO₄, filtered and concentrated under reduced pressure to give a residue. The residue was purified by flash silica gel chromatography (40 g Silica Flash Column, Eluent of 0˜100% Ethyl acetate/Petroleum ether gradient @ 100 mL/min) to give 1-methyl-4-(4-nitropyrazol-1-yl) piperidine (910 mg, 4.3 mmol, 6.3% yield) as yellow oil.

To a solution of 1-methyl-4-(4-nitropyrazol-1-yl) piperidine (300 mg, 1.4 mmol, 1 eq) in MeOH (3 mL) was added Pd/C (150 mg, 10% purity) under N₂ atmosphere. The suspension was degassed and purged with H₂ for 3 times. The mixture was stirred under H₂ (15 Psi.) at 20° C. for 2 hr. LC-MS showed desired compound was detected. The reaction mixture was filtered and concentrated under reduced pressure to give 1-(1-methyl-4-piperidyl)pyrazol-4-amine (237.4 mg, crude) as a gray solid.

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (300 mg, 617.8 μmol, 1 eq) in dioxane (10 mL) was added TFA (140.8 mg, 1.2 mmol, 91.4 μL, 2 eq) and 1-(1-methyl-4-piperidyl)pyrazol-4-amine (167 mg, 926.7 μmol, 1.5 eq). The mixture was stirred at 100° C. for 12 hr. LC-MS showed desired compound was detected. The reaction mixture was concentrated under reduced pressure to remove solvent. The residue was purified by prep-HPLC (TFA condition) column: Phenomenex luna C18 100×40 mm× 5 μm; mobile phase: [water (TFA)-ACN];B %: 25%-55%, 8 min to give tert-butyl 8-methyl-4-[[8-methyl-2-[1-(1-methyl-4-piperidyl)pyrazol-4-yl]amino]-7-oxo-pyrido[2,3-d]pyrimidin-6-yl]-2,3-dihydroquinoxaline-1-carboxylate (73 mg, 124.6 μmol, 20.17% yield) as a yellow solid.

A mixture of tert-butyl 8-methyl-4-[8-methyl-2-[[1-(1-methyl-4-piperidyl)pyrazol-4-yl]amino]-7-oxo-pyrido[2,3-d]pyrimidin-6-yl]-2,3-dihydroquinoxaline-1-carboxylate (73 mg, 124.6 μmol, 1 eq) in HCl/EtOAc (2 mL 4 M) was stirred at 20° C. for 1 hr. LC-MS showed desired compound was detected. The reaction mixture was concentrated under reduced pressure to give 8-methyl-6-(5-methyl-3,4-dihydro-2H-quinoxalin-1-yl)-2-[[1-(1-methyl-4-piperidyl)pyrazol-4-yl]amino]pyrido[2,3-d]pyrimidin-7-one (70 mg, crude, HCl) as a yellow solid.

To a solution of 8-methyl-6-(5-methyl-3,4-dihydro-2H-quinoxalin-1-yl)-2-[[1-(1-methyl-4-piperidyl)pyrazol-4-yl]amino]pyrido[2,3-d]pyrimidin-7-one (60 mg, 123.5 μmol, 1 eq) in DCM (1 mL) was added dropwise TEA (37.5 mg, 370.6 μmol, 51.6 μL, 3 eq) and prop-2-enoyl chloride (11.1 mg, 123.5 μmol, 10 μL, 1 eq) in DCM (0.1 mL) at 0° C. The resulting mixture was stirred at 0° C. for 30 min. LC-MS showed desired compound was detected. The reaction mixture was concentrated under N₂ to remove solvent. The residue was purified by prep-HPLC (TFA condition) column: Phenomenex Luna C18 100×30 mm× 5 μm; mobile phase: [water (TFA)-ACN];B %: 15%-45%, 8 min to give 8-methyl-2-[[1-(1-methyl-4-piperidyl)pyrazol-4-yl]amino]-6-(5-methyl-4-prop-2-enoyl-2,3-dihydroquinoxalin-1-yl)pyrido[2,3-d]pyrimidin-7-one (12.2 mg, 22.5 μmol, 18.2% yield, 99% purity) as a yellow solid.

### Procedure for Preparation of Compound 046

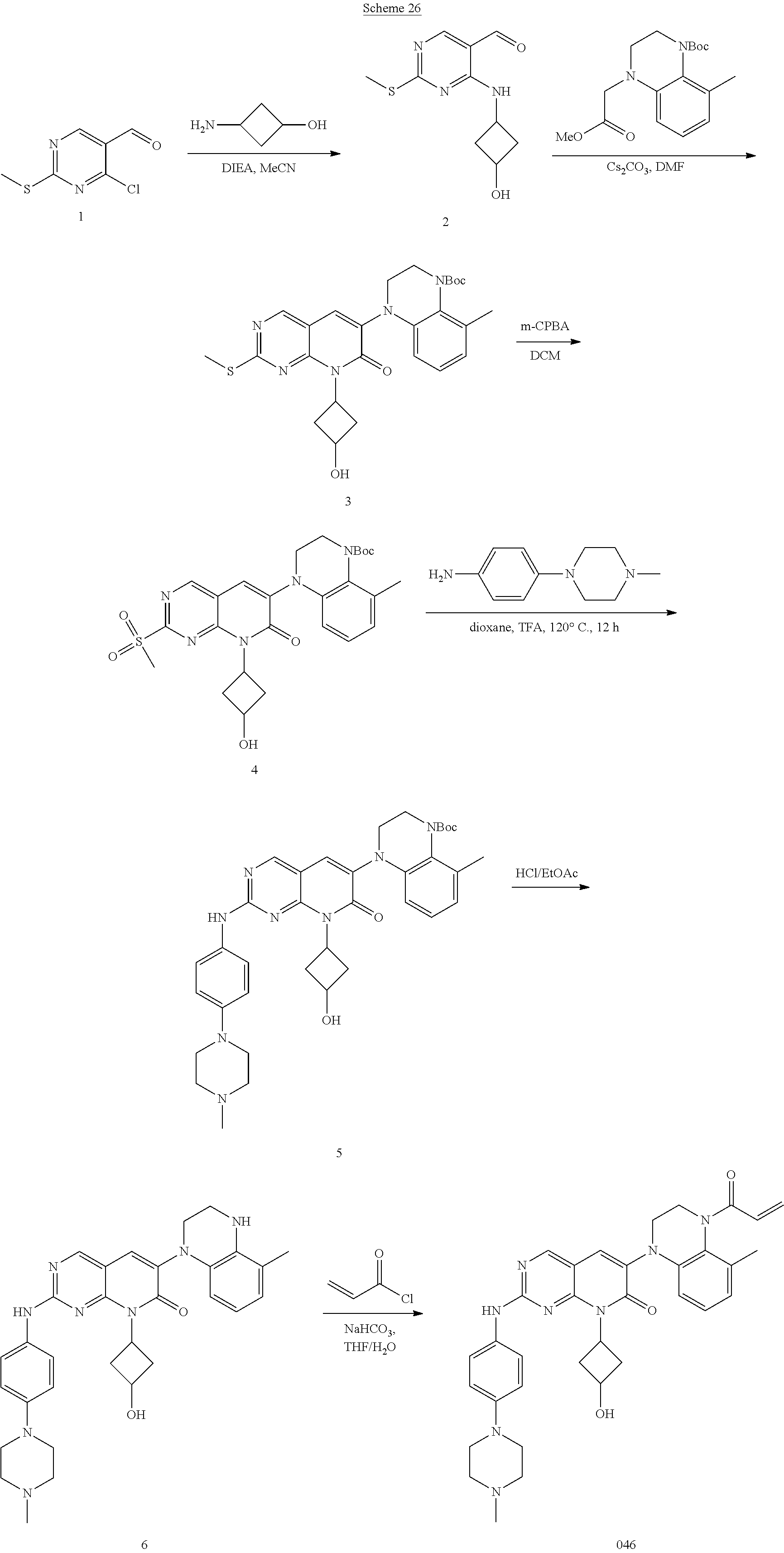

To a solution of 4-chloro-2-methylsulfanyl-pyrimidine-5-carbaldehyde (1 g, 5.3 mmol, 1 eq) in MeCN (20 mL) was added DIEA (2.1 g, 15.9 mmol, 2.8 mL, 3 eq) and 3-aminocyclobutanol (461.8 mg, 5.3 mmol, 1 eq). The mixture was stirred at 25° C. for 2 hr. LC-MS showed desired compound was detected. The reaction mixture was concentrated under reduced pressure to remove solvent. The residue was purified by flash silica gel chromatography (12 g Silica Flash Column, Eluent of 0˜30% Ethyl acetate/Petroleum ether gradient @ 100 mL/min) to give 4-[(3-hydroxycyclobutyl)amino]-2-methylsulfanyl-pyrimidine-5-carbaldehyde (664.2 mg, 2.8 mmol, 52.4% yield) as a white solid.

To a solution of 4-[(3-hydroxycyclobutyl)amino]-2-methylsulfanyl-pyrimidine-5-carbaldehyde (700 mg, 2.9 mmol, 1 eq) in DMF (10 mL) was added Cs₂CO₃ (2.9 g, 8.8 mmol, 3 eq) and tert-butyl 4-(2-ethoxy-2-oxo-ethyl)-8-methyl-2,3-dihydroquinoxaline-1-carboxylate (978.2 mg, 2.9 mmol, 1 eq). The mixture was stirred at 20° C. for 12 hr. LC-MS showed desired compound was detected. The residue was diluted with H₂O 30 mL and extracted with ethyl acetate (30 mL× 3). The combined organic layers were washed with brine (25 mL× 1), dried over Na₂SO₄, filtered and concentrated under reduced pressure to give a residue. The residue was purified by flash silica gel chromatography (20 g Silica Flash Column, Eluent of 0˜35% Ethyl acetate/Petroleum ether gradient @ 120 mL/min) to give tert-butyl 4-[8-(3-hydroxycyclobutyl)-2-methylsulfanyl-7-oxo-pyrido[2,3-d]pyrimidin-6-yl]-8-methyl-2,3-dihydroquinoxaline-1-carboxylate (665 mg, 1.3 mmol, 44.6% yield) as a yellow oil.

To a solution of tert-butyl 4-[8-(3-hydroxycyclobutyl)-2-methylsulfanyl-7-oxo-pyrido[2,3-d]pyrimidin-6-yl]-8-methyl-2,3-dihydroquinoxaline-1-carboxylate (1.1 g, 2.2 mmol, 1 eq) in DCM (15 mL) was added m-CPBA (657.3 mg, 3.2 mmol, 85% purity, 1.5 eq) at 0° C. The mixture was stirred at 25° C. for 3 hr. LC-MS showed desired compound was detected. The reaction mixture was partitioned between Na₂SO_{3 30} ml and dichloromethane (20×3 mL). The organic phase was separated, washed with NaHCO₃ (50 mL×3), dried over Na₂SO₄, filtered and concentrated under reduced pressure to give a residue to give tert-butyl 4-[8-(3-hydroxycyclobutyl)-2-methylsulfonyl-7-oxo-pyrido[2,3-d]pyrimidin-6-yl]-8-methyl-2,3-dihydroquinoxaline-1-carboxylate (1 g, crude) as a yellow solid.

To a solution of tert-butyl 4-[8-(3-hydroxycyclobutyl)-2-methylsulfonyl-7-oxo-pyrido[2,3-d]pyrimidin-6-yl]-8-methyl-2,3-dihydroquinoxaline-1-carboxylate (206.1 mg, 380.5 μmol, 1 eq) in dioxane (2 mL) was added TFA (65.1 mg, 570.8 μmol, 42.4 μL, 1.5 eq) and 4-(4-methylpiperazin-1-yl)aniline (123.7 mg, 646.9 μmol, 1.7 eq). The mixture was stirred at 120° C. for 12 hr (batch×2). LC-MS showed desired compound was detected. The reaction mixture was concentrated under reduced pressure to remove solvent. The residue was purified by prep-HPLC (TFA condition) column: Phenomenex luna C18 100×40 mm×5 μm; mobile phase: [water (TFA)-ACN];B %: 25%-55%, 8 min to give tert-butyl 4-[8-(3-hydroxycyclobutyl)-2-[4-(4-methylpiperazin-1-yl)anilino]-7-oxo-pyrido[2,3-d]pyrimidin-6-yl]-8-methyl-2,3-dihydroquinoxaline-1-carboxylate (250 mg, 382.98 μmol, 50.32% yield) as a yellow solid.

A mixture of tert-butyl 4-[8-(3-hydroxycyclobutyl)-2-[4-(4-methylpiperazin-1-yl)anilino]-7-oxo-pyrido[2,3-d]pyrimidin-6-yl]-8-methyl-2,3-dihydroquinoxaline-1-carboxylate (80 mg, 122.6 μmol, 1 eq) in HCl/EtOAc (1 mL,4 M) and EtOAc (1 mL) was stirred at 15° C. for 1 hr. LC-MS showed desired compound was detected. The reaction mixture was concentrated under reduced pressure to remove solvent to give 8-(3-hydroxycyclobutyl)-6-(5-methyl-3,4-dihydro-2H-quinoxalin-1-yl)-2-[4-(4-methylpiperazin-1-yl)anilino]pyrido[2,3-d]pyrimidin-7-one (80 mg, crude) as a yellow solid.

To a solution of 8-(3-hydroxycyclobutyl)-6-(5-methyl-3,4-dihydro-2H-quinoxalin-1-yl)-2-[4-(4-methylpiperazin-1-yl)anilino]pyrido[2,3-d]pyrimidin-7-one (80 mg, 144.8 μmol, 1 eq) in H₂O (1 mL) and THF (1 mL) was added NaHCO₃ (48.6 mg, 579.0 μmol, 22.5 μL, 4 eq) and prop-2-enoyl chloride (13.1 mg, 144.8 μmol, 11.8 μL, 1 eq) at 0° C. The mixture was stirred at 0° C. for 30 min. LC-MS showed desired compound was detected. The reaction mixture was concentrated under reduced pressure to remove solvent. The residue was purified by prep-HPLC (TFA condition) column: Phenomenex luna C18 100×40 mm×5 μm; mobile phase:

[water (TFA)-ACN];B %: 20%-50%, 8 min to give 8-(3-hydroxycyclobutyl)-2-[4-(4-methylpiperazin-1-yl)anilino]-6-(5-methyl-4-prop-2-enoyl-2,3-dihydroquinoxalin-1-yl)pyrido[2,3-d]pyrimidin-7-one (31.45 mg, 43.64 μmol, 30.15% yield, 100% purity, TFA) as a yellow solid.

### Procedure for Preparation of Compound 052

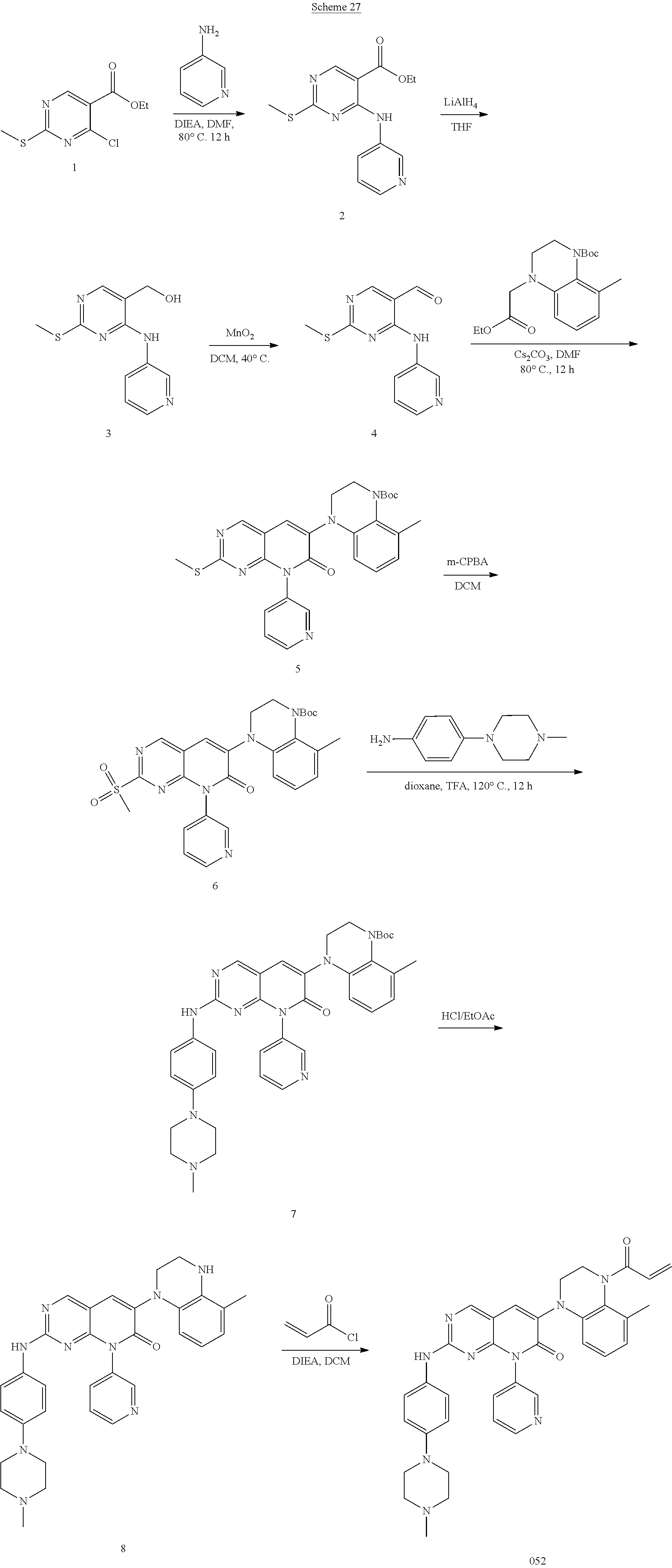

To a solution of ethyl 4-chloro-2-methylsulfanyl-pyrimidine-5-carboxylate (30 g, 128.9 mmol, 1 eq) in DMF (150 mL) was added DIEA (60.1 g, 465.0 mmol, 81.0 mL, 3.6 eq) and pyridin-3-amine (14.6 g, 154.7 mmol, 1.2 eq). The mixture was stirred at 80° C. for 12 hr. (batch×3). LC-MS showed desired compound was detected. The reaction was concentrated under reduce pressure. The residue was purified by flash silica gel chromatography (220 g Silica Flash Column, Eluent of 0˜100% Ethyl acetate/Petroleum ether gradient @ 150 mL/min) to give compound ethyl 2-methylsulfanyl-4-(3-pyridylamino)pyrimidine-5-carboxylate (8 g, 27.6 mmol, 7.1% yield) as a yellow solid.

Ethyl 2-methylsulfanyl-4-(3-pyridylamino)pyrimidine-5-carboxylate (4.8 g, 16.4 mmol, 1 eq) in THF (20 mL) was dropwise added LiAIH4 (1.2 g, 32.8 mmol, 2 eq) in THF (20 mL). The mixture was stirred at 0° C. for 2 hr. LC-MS showed desired compound was detected. Sat·MgSO₄ (1.24 mL) was added to the mixture at 0° C., and MgSO₄ (400 mg) was added, then filtered and the filtrate was concentrated under reduce pressure to give compound [2-methylsulfanyl-4-(3-pyridylamino)pyrimidin-5-yl]methanol (10 g, crude) as a white solid.

To a solution of [2-methylsulfanyl-4-(3-pyridylamino)pyrimidin-5-yl]methanol (4.2 g, 16.9 mmol, 1 eq) in DCM (80 mL) was added MnO₂ (14.7 g, 169.2 mmol, 10 eq). The mixture was stirred at 40° C. for 12 hr (batch×2). LC-MS showed desired compound was detected. The reaction was filtered to remove the undissolved substance, then the filtrate was concentrated under reduce pressure to give a residue. The residue was purified by flash silica gel chromatography (20 g Silica Flash Column, Eluent of 0˜100% Ethyl acetate/Petroleum ether gradient @ 80 mL/min) to give compound 2-methylsulfanyl-4-(3-pyridylamino)pyrimidine-5-carbaldehyde (1.5 g, 6.1 mmol, 18.0% yield) as a white solid.

To a solution of 2-methylsulfanyl-4-(3-pyridylamino)pyrimidine-5-carbaldehyde (1.4 g, 5.8 mmol, 1 eq) in DMF (15 mL) was added Cs₂CO₃ (5.7 g, 17.4 mmol, 3 eq) and tert-butyl 4-(2-ethoxy-2-oxo-ethyl)-8-methyl-2,3-dihydroquinoxaline-1-carboxylate (1.9 g, 5.8 mmol, 1 eq). The mixture was stirred at 80° C. for 12 hr. LC-MS showed desired compound was detected. The reaction mixture was quenched by addition H₂O (10 mL) at 25° C., and then diluted with ethyl acetate (10 mL) and extracted with ethyl acetate (20 mL× 3). The combined organic layers were washed with brine (15 mL), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 80 mL/min) to give compound tert-butyl 8-methyl-4-[2-methylsulfanyl-7-oxo-8-(3-pyridyl)pyrido[2,3-d]pyrimidin-6-yl]-2,3-dihydroquinoxaline-1-carboxylate (984 mg, 1.9 mmol, 32.9% yield) as a white solid.

To a solution of tert-butyl 8-methyl-4-[2-methylsulfanyl-7-oxo-8-(3-pyridyl)pyrido[2,3-d]pyrimidin-6-yl]-2,3-dihydroquinoxaline-1-carboxylate (194 mg, 375.5 μmol, 1 eq) in DCM (2 mL) was added m-CPBA (91.5 mg, 450.6 μmol, 85% purity, 1.2 eq) at 0° C. The mixture was stirred at 25° C. for 6 hr. LC-MS showed desired compound was detected. The reaction mixture was quenched by addition sat. Na₂SO₃ (20 mL) at 25° C., then diluted with dichloromethane (15 mL) and extracted with dichloromethane (20 mL× 3) and washed with sat. NaHCO₃ (10 mL× 3), dried over Na₂SO₄, filtered and concentrated under reduced pressure to give compound tert-butyl 8-methyl-4-[2-methylsulfinyl-7-oxo-8-(3-pyridyl)pyrido[2,3-d]pyrimidin-6-yl]-2,3-dihydroquinoxaline-1-carboxylate (220 mg, crude) as a white solid.

To a solution of tert-butyl 8-methyl-4-[2-methylsulfinyl-7-oxo-8-(3-pyridyl)pyrido[2,3-d]pyrimidin-6-yl]-2,3-dihydroquinoxaline-1-carboxylate (208 mg, 390.5 μmol, 1 eq) in dioxane (2 mL) was added TFA (66.8 mg, 585.8 μmol, 43.5 L, 1.5 eq) and 4-(4-methylpiperazin-1-yl)aniline (89.6 mg, 468.6 μmol, 1.2 eq). The mixture was stirred at 120° C. for 12 hr. LC-MS showed desired compound was detected. The residue was purified by prep-HPLC (TFA condition; column: Phenomenex luna C18 100×40 mm× 5 μm; mobile phase: [H₂O (0.1% TFA)-ACN]; gradient: 30%-60% B over 8.0 min) to give compound tert-butyl 8-methyl-4-[2-[4-(4-methylpiperazin-1-yl)anilino]-7-oxo-8-(3-pyridyl)pyrido[2,3-d]pyrimidin-6-yl]-2,3-dihydroquinoxaline-1-carboxylate (130 mg, 169.5 μmol, 43.4% yield, 86% purity) as a white solid.

A solution of tert-butyl 8-methyl-4-[2-[4-(4-methylpiperazin-1-yl)anilino]-7-oxo-8-(3-pyridyl)pyrido[2,3-d]pyrimidin-6-yl]-2,3-dihydroquinoxaline-1-carboxylate (124 mg, 187.9 μmol, 1 eq) in HCl/EtOAc (2 mL, 4 M) was stirred at 25° C. for 1 hr. LC-MS showed desired compound was detected. The reaction was concentrated under reduce pressure to give compound 6-(5-methyl-3,4-dihydro-2H-quinoxalin-1-yl)-2-[4-(4-methylpiperazin-1-yl)anilino]-8-(3-pyridyl)pyrido[2,3-d]pyrimidin-7-one (120 mg, crude, HCl) as a white solid.

To a solution of 6-(5-methyl-3,4-dihydro-2H-quinoxalin-1-yl)-2-[4-(4-methylpiperazin-1-yl)anilino]-8-(3-pyridyl)pyrido[2,3-d]pyrimidin-7-one (60 mg, 100.7 μmol, 1 eq, HCl) in DCM (2 mL) was added DIEA to adjust the pH to 7-8, then DIEA (39.0 mg, 301.9 μmol, 52.6 μL, 3 eq) was added to the mixture, the mixture was stirred at 0° C. for 1 hr. LC-MS showed reactant was remained, prop-2-enoyl chloride (4.6 mg, 50.3 μmol, 4.1 μL, 0.5 eq) was added to the mixture, the mixture was stirred at 0° C. for 0.5 hr. LC-MS showed desired compound was detected. The residue was purified by prep-HPLC (TFA condition: column: Phenomenex luna C18 100×40 mm× 5 μm; mobile phase: [H₂O (0.1% TFA)-ACN]; gradient: 10%-40% B over 8.0 min).to give compound 2-[4-(4-methylpiperazin-1-yl)anilino]-6-(5-methyl-4-prop-2-enoyl-2,3-dihydroquinoxalin-1-yl)-8-(3-pyridyl)pyrido[2,3-d]pyrimidin-7-one (10.6 mg, 17.1 μmol, 17.0% yield, 98.9% purity) as a white solid.

### Procedure for Preparation of Compound 065

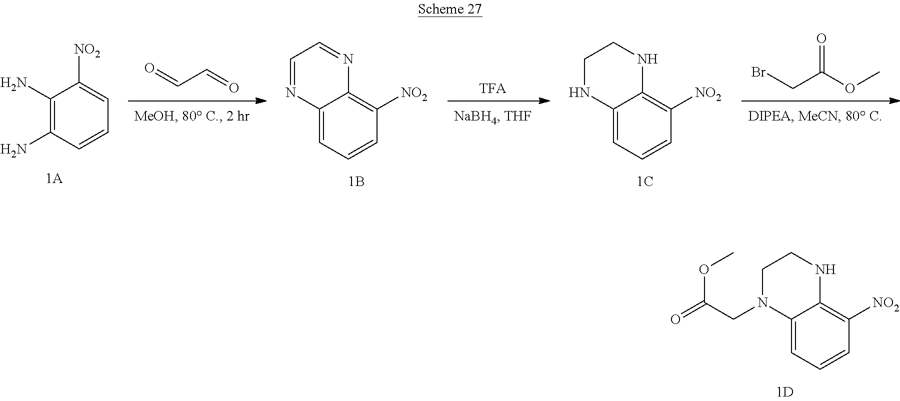

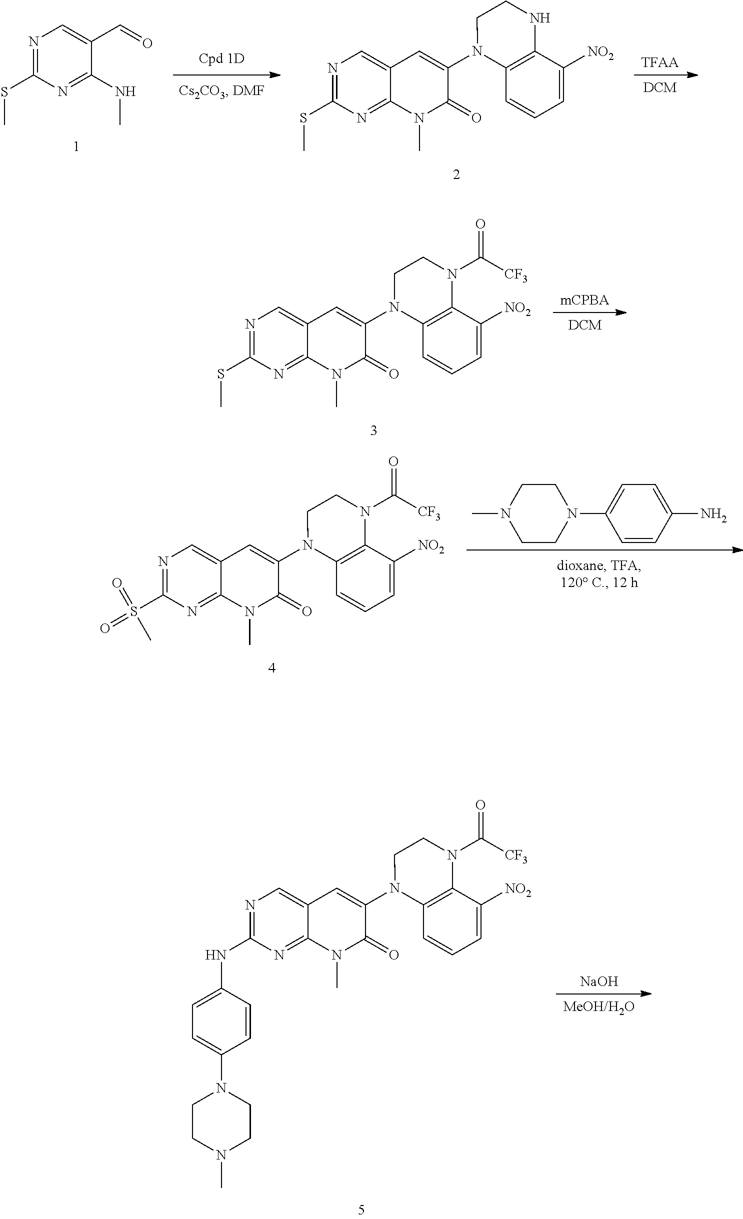

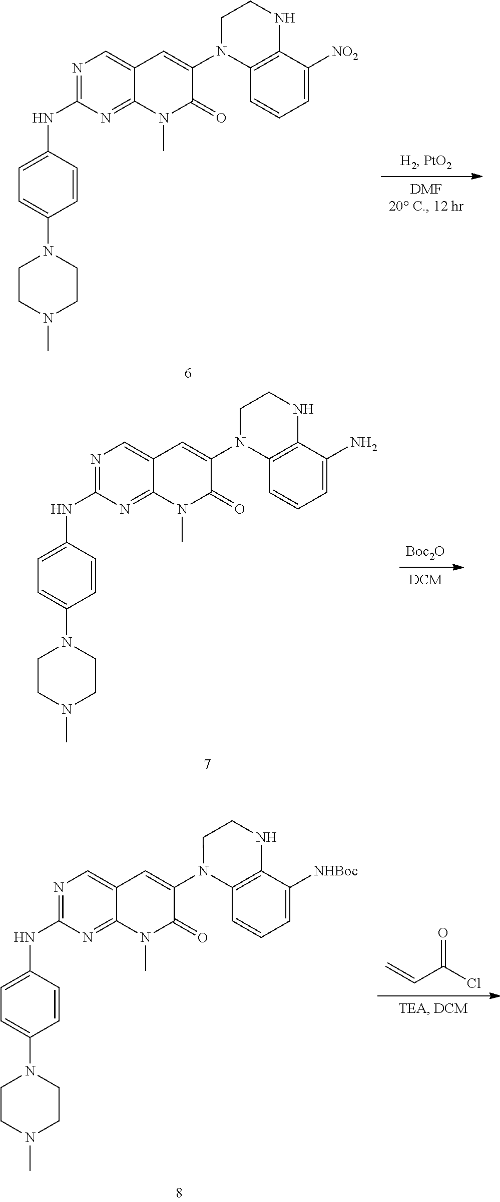

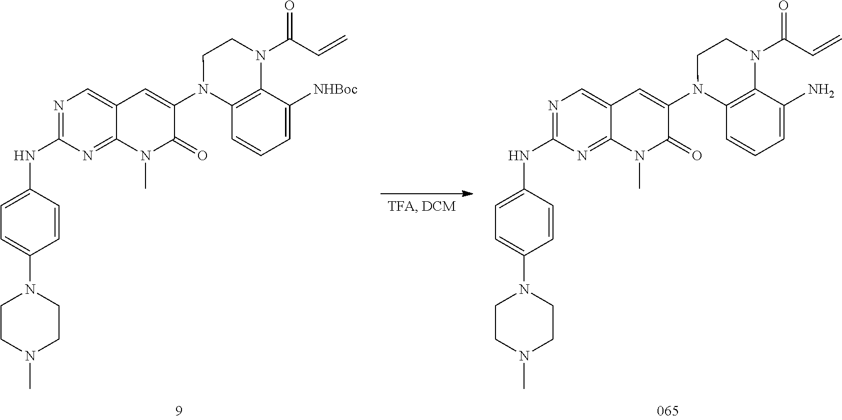

To a solution of 3-nitrobenzene-1,2-diamine (20 g, 130.6 mmol, 1 eq) in EtOH (320 mL) was added oxaldehyde (47.4 g, 326.5 mmol, 42.6 mL, 2.5 eq). The mixture was stirred at 80° C. for 2 hr. LC-MS showed desired compound was detected. The reaction mixture was concentrated under reduced pressure to remove solvent. The residue was diluted with H₂O (300 mL) and extracted with DCM (200 mL× 3), the combined organic layers dried over Na₂SO₄, filtered and concentrated under reduced pressure to give 5-nitroquinoxaline (30 g, crude) as a black solid.

To a solution of 5-nitroquinoxaline (5 g, 28.6 mmol, 1 eq) in THF (500 mL) was added NaBH₄ (3.8 g, 99.9 mmol, 3.5 eq) at 0° C. The mixture was stirred at 0° C. for 30 min. Then TFA (5.9 g, 51.4 mmol, 3.8 mL, 1.8 eq) was dropwise added to the mixture at 0° C. The mixture was stirred at 20° C. for 12 hr. LC-MS showed desired compound was detected. The reaction mixture was quenched by addition NH₄CI (800 mL) at 0° C., and then extracted with ethyl acetate (800 mL× 3). The combined organic layers were washed with brine (600 mL× 3), dried over Na₂SO₄, filtered and concentrated under reduced pressure to give a residue. The residue was purified by flash silica gel chromatography (330 g Silica Flash Column, Eluent of 0˜100% Ethyl acetate/Petroleum ether gradient @ 200 mL/min) to give 5-nitro-1,2,3,4-tetrahydroquinoxaline (3.6 g, 20.3 mmol, 11.8% yield) as a black solid.

To a solution of 5-nitro-1,2,3,4-tetrahydroquinoxaline (2 g, 11.2 mmol, 1 eq) in MeCN (30 mL) was added methyl 2-bromoacetate (1.7 g, 11.2 mmol, 1.1 mL, 1 eq) and DIPEA (1.7 g, 13.4 mmol, 2.3 mL, 1.2 eq). The mixture was stirred at 80° C. for 12 hr. LC-MS showed desired compound was detected. The residue was diluted with H₂O (150 mL) and extracted with ethyl acetate (150 mL× 3). The combined organic layers were washed with brine (130 mL× 1), dried over Na₂SO₄, filtered and concentrated under reduced pressure to give a residue. The residue was purified by flash silica gel chromatography (40 g Silica Flash Column, Eluent of 0˜20% Ethyl acetate/Petroleum ether gradient @ 120 mL/min) to give methyl 2-(5-nitro-3,4-dihydro-2H-quinoxalin-1-yl)acetate (2.2 g, 8.8 mmol, 78.5% yield) as a black solid.

To a solution of methyl 2-[5-nitro-4-(2,2,2-trifluoroacetyl)-2,3-dihydroquinoxalin-1-yl]acetate (1.2 g, 3.46 mmol, 1 eq) in DMF (20 mL) was added Cs₂CO₃ (3.4 g, 10.4 mmol, 3 eq) and 4-(methylamino)-2-methylsulfanyl-pyrimidine-5-carbaldehyde (633.2 mg, 3.5 mmol, 1 eq). The mixture was stirred at 60° C. for 12 hr. LC-MS showed desired compound was detected. The residue was diluted with NaHCO₃ (500 mL) and extracted with ethyl acetate (500 mL×3).

The combined organic layers were washed with brine (300 mL×1), dried over Na₂SO₄, filtered and concentrated under reduced pressure to give tert-butyl 4-(1-cyclopropyl-7-methylsulfanyl-2-oxo-4H-pyrimido[4,5-d]pyrimidin-3-yl)-4-deuterio-2,3-dihydroquinoline-1-carboxylate (1 g, crude) as a yellow solid.

To a solution of 8-methyl-2-methylsulfanyl-6-(5-nitro-3,4-dihydro-2H-quinoxalin-1-yl)pyrido[2,3-d]pyrimidin-7-one (300 mg, 780.41 μmol, 1 eq) in TFAA (5 mL) was added DCM (0.5 mL). The mixture was stirred at 20° C. for 12 hr. LC-MS showed desired compound was detected. The reaction mixture was concentrated under reduced pressure to remove solvent. The residue was diluted with NaHCO₃ (40 mL) and extracted with ethyl acetate (40 mL×3). The combined organic layers were washed with brine (30 mL×1), dried over Na₂SO₄, filtered and concentrated under reduced pressure to give 8-methyl-2-methylsulfanyl-6-[5-nitro-4-(2,2,2-trifluoroacetyl)-2,3-dihydroquinoxalin-1-yl]pyrido[2,3-d]pyrimidin-7-one (389 mg, crude) as a

To a solution of 8-methyl-2-methylsulfanyl-6-[5-nitro-4-(2,2,2-trifluoroacetyl)-2,3-dihydroquinoxalin-1-yl]pyrido[2,3-d]pyrimidin-7-one (1.2 g, 2.4 mmol, 1 eq) in DCM (5 mL) was added m-CPBA (882.4 mg, 4.4 mmol, 85% purity, 1.8 eq) at 0° C. The mixture was stirred at 20° C. for 12 hr. LC-MS showed desired compound was detected. The residue was diluted with Na₂SO₃ (150 mL) and extracted with dichloromethane (150 mL× 3). The combined organic layers were washed with NaHCO₃ (100 mL× 1), dried over Na₂SO₄, filtered and concentrated under reduced pressure to give 8-methyl-2-methylsulfonyl-6-[5-nitro-4-(2,2,2-trifluoroacetyl)-2,3-dihydroquinoxalin-1-yl]pyrido[2,3-d]pyrimidin-7-one (1.2 g, crude) as an orange solid.

To a solution of 8-methyl-2-methylsulfonyl-6-[5-nitro-4-(2,2,2-trifluoroacetyl)-2,3-dihydroquinoxalin-1-yl]pyrido[2,3-d]pyrimidin-7-one (200 mg, 390.3 μmol, 1 eq) in dioxane (2 mL) was added TFA (66.7 mg, 585.5 μmol, 43.5 μL, 1.5 eq) and 4-(4-methylpiperazin-1-yl)aniline (89.6 mg, 468.4 μmol, 1.2 eq). The mixture was stirred at 120° C. for 12 hr. LC-MS showed desired compound was detected. The reaction mixture was concentrated under reduced pressure to remove solvent. The residue was purified by prep-HPLC (TFA condition; column: Phenomenex luna C18 100×40 mm×5 μm; mobile phase: [H₂O (0.1% TFA)-ACN]; gradient: 20%-50% B over 8.0 min) and prep-HPLC (TFA condition; column: Phenomenex luna

C18 100×40 mm× 5 μm; mobile phase: [H₂O (0.1% TFA)-ACN]; gradient: 20%-50% B over 8.0 min) to give 8-methyl-2-[4-(4-methylpiperazin-1-yl)anilino]-6-[5-nitro-4-(2,2,2-trifluoroacetyl)-2,3-dihydroquinoxalin-1-yl]pyrido[2,3-d]pyrimidin-7-one (560 mg, 898.0 μmol, 38.4% yield) as a brown solid.

To a solution of 8-methyl-2-[4-(4-methylpiperazin-1-yl)anilino]-6-[5-nitro-4-(2,2,2-trifluoroacetyl)-2,3-dihydroquinoxalin-1-yl]pyrido[2,3-d]pyrimidin-7-one (300 mg, 481.1 μmol, 1 eq) in MeOH (3 mL) and H₂O (1 mL) was added NaOH(96.2 mg, 2.4 mmol, 5 eq). The mixture was stirred at 50° C. for 1 hr. LC-MS showed desired compound was detected. The residue was diluted with H₂O 20 mL and extracted with dichloromethane (50 mL×3). The combined organic layers were washed with brine (10 mL× 1), dried over Na₂SO₄, filtered and concentrated under reduced pressure to give 8-methyl-2-[4-(4-methylpiperazin-1-yl)anilino]-6-(5-nitro-3,4-dihydro-2H-quinoxalin-1-yl)pyrido[2,3-d]pyrimidin-7-one (180 mg, crude) as an orange solid.

To a solution of PtO2 (50 mg) in DMF (2 mL) was added 8-methyl-2-[4-(4-methylpiperazin-1-yl)anilino]-6-(5-nitro-3,4-dihydro-2H-quinoxalin-1-yl)pyrido[2,3-d]pyrimidin-7-one (100 mg, 189.6 μmol, 1 eq) under N₂ atmosphere. The suspension was degassed and purged with H₂ for 3 times. The mixture was stirred under H₂ (15 psi) at 20° C. for 12 hr. LC-MS showed desired compound was detected. The reaction mixture was filtered and concentrated under reduced pressure to give 6-(5-amino-3,4-dihydro-2H-quinoxalin-1-yl)-8-methyl-2-[4-(4-methylpiperazin-1-yl)anilino]pyrido[2,3-d]pyrimidin-7-one (76 mg, crude) as a black solid.

To a solution of 6-(5-amino-3,4-dihydro-2H-quinoxalin-1-yl)-8-methyl-2-[4-(4-methylpiperazin-1-yl)anilino]pyrido[2,3-d]pyrimidin-7-one (76 mg, 152.7 μmol, 1 eq) in DCM (2 mL) was added Boc₂O (33.3 mg, 152.7 μmol, 35.1 μL, 1 eq). The mixture was stirred at 20° C. for 12 hr. LC-MS showed desired compound was detected. The reaction mixture was concentrated under reduced pressure to remove solvent. The residue was purified by prep-HPLC (FA condition; column: Phenomenex Luna C18 100×30 mm× 5 μm; mobile phase: [H₂O (0.2% FA)-ACN]; gradient: 15%-50% B over 8.0 min) to give tert-butyl N-[1-[8-methyl-2-[4-(4-methylpiperazin-1-yl)anilino]-7-oxo-pyrido[2,3-d]pyrimidin-6-yl]-3,4-dihydro-2H-quinoxalin-5-yl]carbamate (30 mg, 50.19 μmol, 32.86% yield) as a yellow solid.

To a solution of tert-butyl N-[1-[8-methyl-2-[4-(4-methylpiperazin-1-yl)anilino]-7-oxo-pyrido[2,3-d]pyrimidin-6-yl]-3,4-dihydro-2H-quinoxalin-5-yl]carbamate (30 mg, 50.1 μmol, 1 eq) in DCM (1 mL) was added TEA (15.2 mg, 150.6 μmol, 21.0 μL, 3 eq) and prop-2-enoyl chloride (6.8 mg, 75.3 μmol, 6.1 μL, 1.5 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition; column: Phenomenex Luna C18 100×30 mm×5 μm; mobile phase: [H₂O (0.2% FA)-ACN]; gradient: 15%-45% B over 8.0 min) to give tert-butyl N-[1-[8-methyl-2-[4-(4-methylpiperazin-1-yl)anilino]-7-oxo-pyrido[2,3-d]pyrimidin-6-yl]-4-prop-2-enoyl-2,3-dihydroquinoxalin-5-yl]carbamate (25 mg, 38.36 μmol, 76.42% yield) as a yellow solid.

To a solution of tert-butyl N-[1-[8-methyl-2-[4-(4-methylpiperazin-1-yl)anilino]-7-oxo-pyrido[2,3-d]pyrimidin-6-yl]-4-prop-2-enoyl-2,3-dihydroquinoxalin-5-yl]carbamate (25 mg, 38.4 μmol, 1 eq) in DCM (1 mL) was added TFA (0.1 mL). The mixture was stirred at 20° C. for 1 hr. LC-MS showed desired compound was detected. The reaction mixture was concentrated under reduced pressure to remove solvent. The residue was purified by prep-HPLC (FA condition; column: Phenomenex Luna C18 100×30 mm× 5 μm; mobile phase: [H₂O (0.2% FA)-ACN]; gradient: 1%-25% B over 8.0 min) to give 6-(5-amino-4-prop-2-enoyl-2,3-dihydroquinoxalin-1-yl)-8-methyl-2-[4-(4-methylpiperazin-1-yl)anilino]pyrido[2,3-d]pyrimidin-7-one (8.0 mg, 13.0 μmol, 33.9% yield, 97.5% purity, FA) as a yellow solid.

### Procedure for Preparation of Compound 053

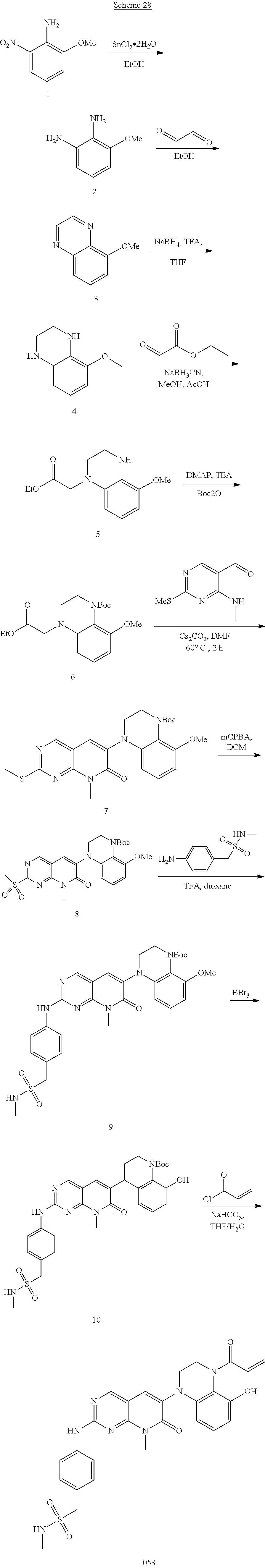

To a solution of 2-methoxy-6-nitro-aniline (10 g, 59.4 mmol, 10.0 mL, 1 eq) in EtOH (200 mL) was added SnCl₂·2H₂O (67.1 g, 297.3 mmol, 5 eq). The mixture was stirred at 75° C. for 3 hr. LC-MS showed desired compound was detected. The residue was diluted with 40% NaOH (200 mL) and extracted with ethyl acetate (300 mL× 3). The combined organic layers were washed with brine (300 mL×1), dried over Na₂SO₄, filtered and concentrated under reduced pressure to give a residue. The residue was purified by flash silica gel chromatography (120 g Silica Flash Column, Eluent of 0˜26% Ethyl acetate/Petroleum ether gradient @ 120 mL/min) to give 3-methoxybenzene-1,2-diamine (14.3 g, 104.0 mmol, 87.5% yield) as a pink oil.

To a solution of 3-methoxybenzene-1,2-diamine (5 g, 36.1 mmol, 1 eq) in EtOH (200 mL) was added oxaldehyde (13.1 g, 90.4 mmol, 11.7 mL, 2.5 eq). The mixture was stirred at 80° C. for 2 hr. LC-MS showed desired compound was detected. The reaction mixture was concentrated under reduced pressure to remove solvent. Compound 5-methoxyquinoxaline (11.5 g, crude) was obtained as a brown oil.

To a solution of 5-methoxyquinoxaline (5.7 g, 35.9 mmol, 1 eq) in THF (150 mL) was added NaBH₄ (6.7 g, 179.4 mmol, 5 eq) at 0° C., the mixture was stirred at 0° C. for 0.5 hr. Then the mixture was added TFA (10·2 g, 89.7 mmol, 6.6 mL, 2.5 eq) at 0° C., the mixture was stirred at 25° C. for 1 hr. LC-MS showed desired compound was detected. The reaction mixture was quenched by addition NH₄CI (400 mL) at 0° C., and then diluted with NH₄CI (200 mL) and extracted with ethyl acetate (400 mL×3). The combined organic layers were washed with brine (500 mL× 1), dried over Na₂SO₄, filtered and concentrated under reduced pressure to give a residue. The residue was purified by flash silica gel chromatography (220 g Silica Flash Column, Eluent of 0˜25% Ethyl acetate/Petroleum ether gradient @ 120 mL/min) to give 5-methoxy-1,2,3,4-tetrahydroquinoxaline (6.5 g, 39.5 mmol, 55.1% yield) as a yellow oil.

To a solution of 5-methoxy-1,2,3,4-tetrahydroquinoxaline (2.5 g, 15·2 mmol, 1 eq) in MeOH (25 mL) was added AcOH(91.4 mg, 1.5 mmol, 87.1 μL, 0.1 eq) and ethyl 2-oxoacetate (3.1 g, 15·2 mmol, 1 eq). The mixture was stirred at 60° C. for 1 hr. Then the mixture was added NaBH₃CN (2.8 g, 45.6 mmol, 3 eq), the mixture was stirred at 60° C. for 1 hr. LC-MS showed desired compound was detected. The residue was diluted with NaHCO₃80 mL and extracted with ethyl acetate (80 mL×3). The combined organic layers were washed with brine (80 mL×1), dried over Na₂SO₄, filtered and concentrated under reduced pressure to give a residue. The residue was purified by flash silica gel chromatography (80 g Silica Flash Column, Eluent of 0˜12% Ethyl acetate/Petroleum ether gradient @ 120 mL/min) to give ethyl 2-(5-methoxy-3,4-dihydro-2H-quinoxalin-1-yl)acetate (2.8 g) as yellow oil.

To a solution of ethyl 2-(5-methoxy-3,4-dihydro-2H-quinoxalin-1-yl)acetate (2.8 g, 11.3 mmol, 1 eq) in Boc₂O (40 mL) was added DMAP (138.1 mg, 1.1 mmol, 0.1 eq) and TEA (5.7 g, 56.5 mmol, 7.8 mL, 5 eq). The mixture was stirred at 80° C. for 12 hr. LC-MS showed desired compound was detected. The reaction mixture was concentrated under reduced pressure to remove solvent. The residue was purified by flash silica gel chromatography (40 g Silica Flash Column, Eluent of 0˜12% Ethyl acetate/Petroleum ether gradient @ 120 mL/min) to give tert-butyl 4-(2-ethoxy-2-oxo-ethyl)-8-methoxy-2,3-dihydroquinoxaline-1-carboxylate (3.7 g, 10.5 mmol, 93.3% yield) as a yellow oil.

To a solution of tert-butyl 4-(2-ethoxy-2-oxo-ethyl)-8-methoxy-2,3-dihydroquinoxaline-1-carboxylate (1.6 g, 4.5 mmol, 1 eq) in DMF (15 mL) was added 4-(methylamino)-2-methylsulfanyl-pyrimidine-5-carbaldehyde (836.6 mg, 4.5 mmol, 1 eq) and Cs₂CO₃ (2.9 g, 9.1 mmol, 2 eq). The mixture was stirred at 60° C. for 2 hr. LC-MS showed desired compound was detected. The reaction mixture was concentrated under reduced pressure to remove solvent. The residue was diluted with H₂O (100 mL) and extracted with ethyl acetate (100 mL× 3). The combined organic layers were washed with brine (150 mL× 3), dried over Na₂SO₄, filtered and concentrated under reduced pressure to give a residue. The residue was purified by flash silica gel chromatography (80 g Silica Flash Column, Eluent of 0˜20% Ethyl acetate/Petroleum ether gradient @ 100 mL/min) to give tert-butyl 8-methoxy-4-(8-methyl-2-methylsulfanyl-7-oxo-pyrido[2,3-d]pyrimidin-6-yl)-2,3-dihydroquinoxaline-1-carboxylate (970 mg, 2.0 mmol, 22.6% yield) as a yellow solid.

To a solution of tert-butyl 8-methoxy-4-(8-methyl-2-methylsulfanyl-7-oxo-pyrido[2,3-d]pyrimidin-6-yl)-2,3-dihydroquinoxaline-1-carboxylate (200 mg, 425.9 μmol, 1 eq) in DCM (3 mL) was added m-CPBA (129.7 mg, 638.9 μmol, 85% purity, 1.5 eq) at 0° C. The resulting mixture was stirred at 25° C. for 2 hr. LC-MS showed desired compound was detected. The reaction mixture was quenched by addition Na₂SO₃ (15 mL), extracted with DCM (10 mL×3). The combined organic layers were washed with NaHCO₃ (10 mL×2), and dried over Na₂SO₄, filtered and concentrated under reduced pressure to give a residue. Compound tert-butyl 8-methoxy-4-(8-methyl-2-methylsulfinyl-7-oxo-pyrido[2,3-d]pyrimidin-6-yl)-2,3-dihydroquinoxaline-1-carboxylate (235.1 mg, crude) was obtained as a red solid.

To a solution of tert-butyl 8-methoxy-4-(8-methyl-2-methylsulfinyl-7-oxo-pyrido[2,3-d]pyrimidin-6-yl)-2,3-dihydroquinoxaline-1-carboxylate (183·2 mg, 377.3 μmol, 1 eq) in dioxane (2 mL) was added 1-(4-aminophenyl)-N-methyl-methanesulfonamide (113.3 mg, 565.9 μmol, 1.5 eq) and TFA (64.5 mg, 565.9 μmol, 42.0 μL, 1.5 eq). The mixture was stirred at 120° C. for 2 hr. LC-MS showed desired compound was detected. The reaction mixture was concentrated under reduced pressure to remove solvent. The residue was purified by prep-HPLC (TFA condition; column: Phenomenex luna C18 100×40 mm×5 μm; mobile phase: [H₂O (0.1% TFA)-ACN]; gradient: 30%-65% B over 8.0 min) to give tert-butyl 8-methoxy-4-[8-methyl-2-[4-(methylsulfamoylmethyl)anilino]-7-oxo-pyrido[2,3-d]pyrimidin-6-yl]-2,3-dihydroquinoxaline-1-carboxylate (118 mg, 153.7 μmol, 8.1% yield, 81% purity) as a yellow solid.

To a solution of tert-butyl 8-methoxy-4-[8-methyl-2-[4-(methylsulfamoylmethyl)anilino]-7-oxo-pyrido[2,3-d]pyrimidin-6-yl]-2,3-dihydroquinoxaline-1-carboxylate (10 mg, 16.0 μmol, 1 eq) in DCM (3 mL) was added BBr₃ (6.0 mg, 24.1 μmol, 2.3 μL, 1.5 eq). The mixture was stirred at 0° C. for 10 min. LC-MS showed desired compound was detected. The reaction mixture was added to NH₃ H₂O to adjust pH to 7-8 at 0° C., and then extracted with dichloromethane (20 ml×3), dried over Na₂SO₄, filtered and concentrated under reduced pressure to give a residue. The residue was purified by prep-HPLC (TFA condition; column: Phenomenex luna C18 100×40 mm×5 μm; mobile phase: [H₂O (0.1% TFA)-ACN];gradient: 5%-35% B over 8.0 min) to give 1-[4-[[6-(5-hydroxy-3,4-dihydro-2H-quinoxalin-1-yl)-8-methyl-7-oxo-pyrido[2,3-d]pyrimidin-2-yl]amino]phenyl]—N-methyl-methanesulfonamide (42.1 mg, 72.1 μmol, 44.8% yield, 87% purity) as a yellow solid.

To a solution of 1-[4-[[6-(5-hydroxy-3,4-dihydro-2H-quinoxalin-1-yl)-8-methyl-7-oxo-pyrido[2,3-d]pyrimidin-2-yl]amino]phenyl]—N-methyl-methanesulfonamide (10.0 mg, 19.7 μmol, 1 eq) in H₂O (0·2 mL) and THF (0.8 mL) was added NaHCO₃ (4.9 mg, 59.1 μmol, 2.3 μL, 3 eq). And prop-2-enoyl chloride (1.7 mg, 19.7 μmol, 1.6 μL, 1 eq) in THF (0.1 mL) was added dropwise at 0° C. The mixture was stirred at 0° C. for 0.5 hr. LC-MS showed desired compound was detected. The reaction mixture was concentrated under reduced pressure to remove solvent. The residue was purified by prep-HPLC (TFA condition; column: Phenomenex luna C18 100×40 mm×5 μm; mobile phase: [H₂O (0.1% TFA)-ACN]; gradient: 15%-45% B over 8.0 min). The residue was purified by prep-HPLC (NH₄HCO₃ condition; column: Waters Xbridge BEH C18 100×30 mm×10 μm; mobile phase: [H₂O (10 mM NH₄HCO₃)-ACN]; gradient: 15%-55% B over 8.0 min) to give 1-[4-[[6-(5-hydroxy-4-prop-2-enoyl-2,3-dihydroquinoxalin-1-yl)-8-methyl-7-oxo-pyrido[2,3-d]pyrimidin-2-yl]amino]phenyl]—N-methyl-methanesulfonamide (8.1 mg, 14.5 μmol, 18.4% yield) as a yellow solid.

### Procedure for Preparation of Compound 073

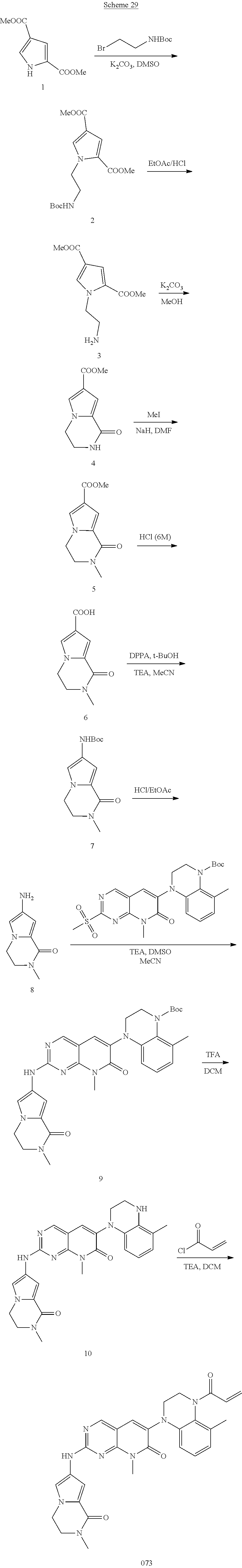

A mixture of dimethyl 1H-pyrrole-2,4-dicarboxylate (1 g, 5.5 mmol, 1 eq), tert-butyl N-(2-bromoethyl) carbamate (3.7 g, 16.4 mmol, 3 eq), K₂CO₃ (2.3 g, 16.4 mmol, 3 eq) in DMSO (200 mL) was degassed and purged with N₂ for 3 times, and then the mixture was stirred at 80° C. for 12 hr under N₂ atmosphere. LC-MS showed desired compound was detected. The mixture was poured into water (100 mL) and then extracted by ethyl acetate (50 mL× 3), the combined organic layers was washed by brine and dried by Na₂SO₄, filtered and the filtrate was concentrated to get a residue. The residue was purified by column chromatography (SiO₂, Petroleum ether/Ethyl acetate=5/1 to 2/1) to give dimethyl 1-[2-(tert-butoxycarbonylamino)ethyl]pyrrole-2,4-dicarboxylate (15 g) as a white solid.

To a solution of dimethyl 1-[2-(tert-butoxycarbonylamino)ethyl]pyrrole-2,4-dicarboxylate (2.5 g, 7.7 mmol, 1 eq) in EtOAc (10 mL) was added HCl/EtOAc (40 mL,4M), the mixture was stirred at 25° C. for 2 hr. LC-MS showed desired compound was detected. The mixture was concentrated to get the crude product dimethyl 1-(2-aminoethyl)pyrrole-2,4-dicarboxylate (8 g, crude, HCl) as a yellow solid.

To a solution of dimethyl 1-(2-aminoethyl)pyrrole-2,4-dicarboxylate (2 g, 7.6 mmol, 1 eq, HCl) in MeOH (30 mL) was added K₂CO₃ (5.3 g, 38.1 mmol, 5 eq) under N₂ atmosphere. The mixture was stirred at 80° C. for 12 hr. LC-MS showed desired compound was detected. The mixture was filtered and the filtrate was concentrated to get the crude product methyl 1-oxo-3,4-dihydro-2H-pyrrolo[1,2-a]pyrazine-7-carboxylate (6 g, crude) as a white solid.

A mixture of methyl 1-oxo-3,4-dihydro-2H-pyrrolo[1,2-a]pyrazine-7-carboxylate (1.1 g, 5.6 mmol, 1 eq) in DMF (150 mL) was degassed and purged with N₂ for 3 times, then NaH (555.1 mg, 13.9 mmol, 60% purity, 2.5 eq) was added, the mixture was stirred at 20° C. for 20 min and then Mel (2.4 g, 16.7 mmol, 1.0 mL, 3 eq) was added the mixture was stirred at 20° C. for 12 hr under N₂ atmosphere. LC-MS showed desired compound was detected. The mixture was quenched by sat. NH₄CI (50 mL) and then extracted by ethyl acetate (50 mL× 3), the combined organic layers was washed by brine (20 mL×3), then the organic layers was dried by Na₂SO₄, filtered and the filtrate was concentrated to get a residue. The residue was purified by column chromatography (SiO₂, Petroleum ether/Ethyl acetate=3/1 to 0/1 to THF/ethyl acetate=1/1) to give the product methyl 2-methyl-1-oxo-3,4-dihydropyrrolo[1,2-a]pyrazine-7-carboxylate (0.9 g, 4.3 mmol, 19.5% yield) as orange oil.

A mixture of methyl 2-methyl-1-oxo-3,4-dihydropyrrolo[1,2-a]pyrazine-7-carboxylate (0.7 g, 3.4 mmol, 1 eq) in HCl (10 mL, 6M) was stirred at 80° C. for 2 hr. The mixture was stirred at 25° C. for 3 hr. LC-MS showed desired compound was detected. The mixture was concentrated to get the product 2-methyl-1-oxo-3,4-dihydropyrrolo[1,2-a]pyrazine-7-carboxylic acid (0.6 g, crude) as an orange solid

To a solution of 2-methyl-1-oxo-3,4-dihydropyrrolo[1,2-a]pyrazine-7-carboxylic acid (100 mg, 515.0 μmol, 1 eq) in MeCN (4 mL) was added DPPA (141.7 mg, 515.0 μmol, 111·2 μL, 1 eq) and TEA (52.1 mg, 515.0 μmol, 71.7 μL, 1 eq), the mixture was stirred at 80° C. for 3 hr, then t-BuOH(2 mL) was added, the mixture was stirred at 80° C. for 12 hr. LC-MS showed desired compound was detected. The mixture was filtered and the filtrate was concentrated to get a residue. The residue was purified by Prep-TLC (SiO₂, Petroleum ether/Ethyl acetate=0/1) to give compound tert-butyl N-(2-methyl-1-oxo-3,4-dihydropyrrolo[1,2-a]pyrazin-7-yl) carbamate (110 mg, 414.6 μmol, 80.51% yield) as a brown solid.

To a solution of tert-butyl N-(2-methyl-1-oxo-3,4-dihydropyrrolo[1,2-a]pyrazin-7-yl) carbamate (170 mg, 640.8 μmol, 1 eq) in EtOAc (1 mL) was added HCl/EtOAc (1 mL, 4M), the mixture was stirred at 25° C. for 30 min. LC-MS showed desired compound was detected. The reaction mixture was concentrated under reduced pressure to get 7-amino-2-methyl-3,4-dihydropyrrolo[1,2-a]pyrazin-1-one (100 mg, crude) as a brown solid.

To a solution of 7-amino-2-methyl-3,4-dihydropyrrolo[1,2-a]pyrazin-1-one (80 mg, 484.3 μmol, 1 eq) in MeCN (1 mL) and DMSO (0.1 mL) was added TEA (66.3 mg, 654.9 μmol, 91·2 μL, 1.35 eq) and tert-butyl 8-methyl-4-(8-methyl-2-methylsulfonyl-7-oxo-pyrido[2,3-d]pyrimidin-6-yl)-2,3-dihydroquinoxaline-1-carboxylate (235·2 mg, 484.3 μmol, 1 eq). The mixture was stirred at 100° C. for 12 hr. LC-MS showed desired compound was detected. The mixture was poured into water (5 mL) and then extracted by ethyl acetate (1 mL× 3), the combined organic layers was dried by Na₂SO₄, filtered and the filtrate was concentrated to get a residue. The residue was purified by flash silica gel chromatography (4 g Silica Flash Column, Eluent of 0˜100% Ethyl acetate/Petroleum ether gradient @ 100 mL/min) to give compound tert-butyl 8-methyl-4-[8-methyl-2-[(2-methyl-1-oxo-3,4-dihydropyrrolo[1,2-a]pyrazin-7-yl)amino]-7-oxo-pyrido[2,3-d]pyrimidin-6-yl]-2,3-dihydroquinoxaline-1-carboxylate (70 mg, 122.7 μmol, 25.3% yield) as an orange solid.

To a solution of tert-butyl 8-methyl-4-[8-methyl-2-[(2-methyl-1-oxo-3,4-dihydropyrrolo[1,2-a]pyrazin-7-yl)amino]-7-oxo-pyrido[2,3-d]pyrimidin-6-yl]-2,3-dihydroquinoxaline-1-carboxylate (50 mg, 87.6 μmol, 1 eq) in DCM (2 mL) was added TFA (230.3 mg, 2.0 mmol, 150.0 μL, 20 eq), the mixture was stirred at 25° C. for 12 hr. LC-MS showed desired compound was detected. The mixture was concentrated to get the crude product 8-methyl-6-(5-methyl-3,4-dihydro-2H-quinoxalin-1-yl)-2-[(2-methyl-1-oxo-3,4-dihydropyrrolo[1,2-a]pyrazin-7-yl)amino]pyrido[2,3-d]pyrimidin-7-one (50 mg, crude, TFA) as a red oil used into the next step without further purification.

To a solution of 8-methyl-6-(5-methyl-3,4-dihydro-2H-quinoxalin-1-yl)-2-[(2-methyl-1-oxo-3,4-dihydropyrrolo[1,2-a]pyrazin-7-yl)amino]pyrido[2,3-d]pyrimidin-7-one (50 mg, 85.5 μmol, 1 eq, TFA) in DCM (2 mL) was added TEA (34.6 mg, 342.1 μmol, 47.6 μL, 4 eq). The mixture was cooled to 0° C., and then prop-2-enoyl chloride (6·2 mg, 68.4 μmol, 5.6 L, 0.8 eq) was added. The mixture was stirred at 20° C. for 1 hr. LC-MS showed desired compound was detected. The mixture was concentrated to get a residue. The residue was purified by prep-HPLC (TFA condition; column: Phenomenex luna C18 100×40 mm×5 μm; mobile phase: [H₂O (0.1% TFA)-ACN]; gradient: 25%-55% B over 8.0 min) to give 8-methyl-2-[(2-methyl-1-oxo-3,4-dihydropyrrolo[1,2-a]pyrazin-7-yl)amino]-6-(5-methyl-4-prop-2-enoyl-2,3-dihydroquinoxalin-1-yl)pyrido[2,3-d]pyrimidin-7-one (6 mg, 11.1 μmol, 13.0% yield, 97·2% purity) as an orange solid.

### Procedure for Preparation of Compound 042

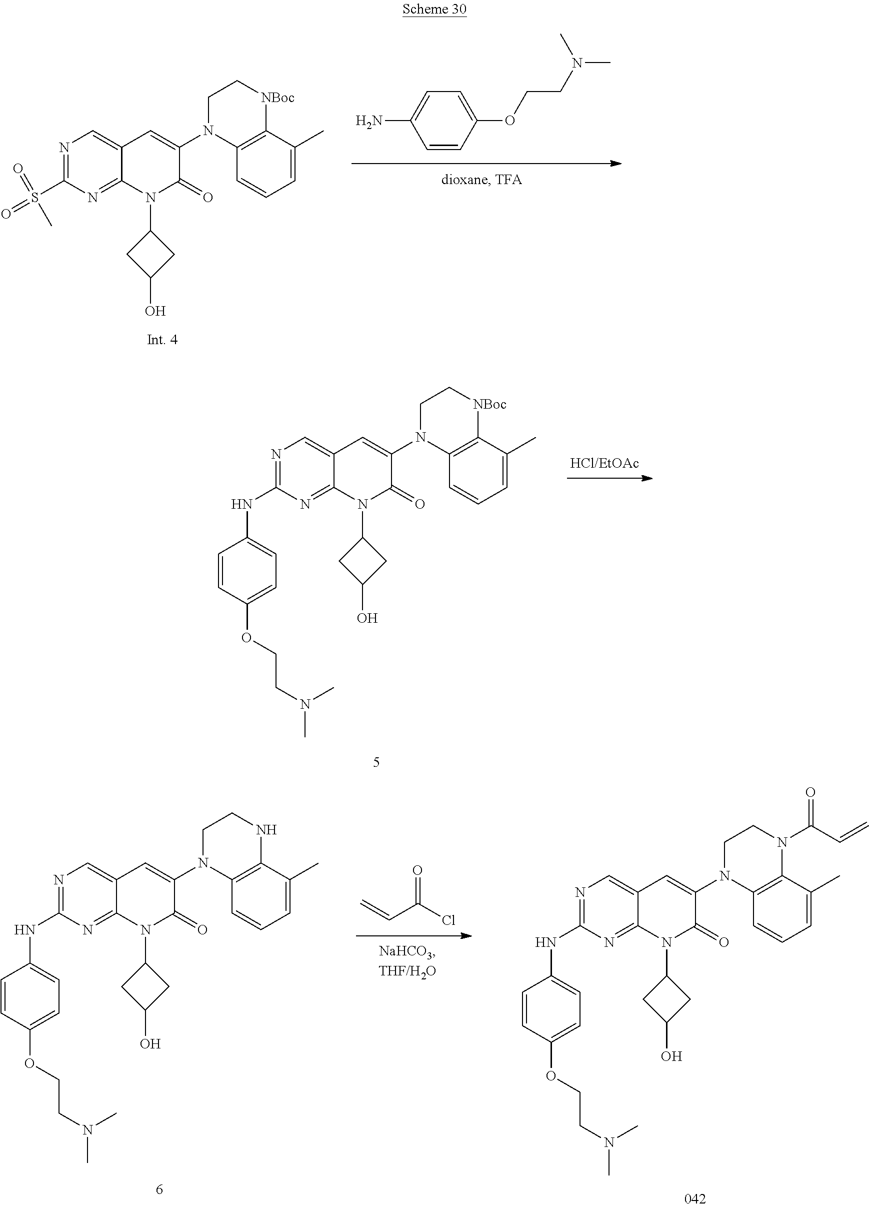

To a solution of tert-butyl 4-[8-(3-hydroxycyclobutyl)-2-methylsulfonyl-7-oxo-pyrido[2,3-d]pyrimidin-6-yl]-8-methyl-2,3-dihydroquinoxaline-1-carboxylate (250 mg, 461.6 μmol, 1 eq) in dioxane (2 mL) was added TFA (78.9 mg, 692.4 μmol, 51.4 μL, 1.5 eq) and 4-[2-(dimethylamino)ethoxy]aniline (166.4 mg, 923·2 μmol, 2 eq). The mixture was stirred at 120° C. for 12 hr. LC-MS showed desired compound was detected. The reaction mixture was concentrated under reduced pressure to remove solvent. The residue was purified by prep-HPLC (TFA condition; column: Phenomenex Luna C18 100×30 mm× 5 μm; mobile phase: [water (TFA)-ACN];B %: 25%-55%, 8 min) to give tert-butyl 4-[2-[4-[2-(dimethylamino)ethoxy]anilino]-8-(3-hydroxycyclobutyl)-7-oxo-pyrido[2,3-d]pyrimidin-6-yl]-8-methyl-2,3-dihydroquinoxaline-1-carboxylate (300 mg, 467.5 μmol, 50.6% yield) as a yellow solid.

A mixture of tert-butyl 4-[2-[4-[2-(dimethylamino)ethoxy]anilino]-8-(3-hydroxycyclobutyl)-7-oxo-pyrido[2,3-d]pyrimidin-6-yl]-8-methyl-2,3-dihydroquinoxaline-1-carboxylate (270 mg, 420.7 μmol, 1 eq) in HCl/EtOAc (2 mL, 4 M) was stirred at 20° C. for 1 hr. LC-MS showed desired compound was detected. The reaction mixture was concentrated under reduced pressure to remove solvent to give 2-[4-[2-(dimethylamino)ethoxy]anilino]-8-(3-hydroxycyclobutyl)-6-(5-methyl-3,4-dihydro-2H-quinoxalin-1-yl)pyrido[2,3-d]pyrimidin-7-one (230 mg, crude) as a yellow solid.

To a solution of 2-[4-[2-(dimethylamino)ethoxy]anilino]-8-(3-hydroxycyclobutyl)-6-(5-methyl-3,4-dihydro-2H-quinoxalin-1-yl)pyrido[2,3-d]pyrimidin-7-one (100 mg, 184.6 μmol, 1 eq) in THF (2 mL)/H₂O (1 mL) was added NaHCO₃ (46.5 mg, 553.9 μmol, 21.6 μL, 3 eq) and prop-2-enoyl chloride (16.7 mg, 184.6 μmol, 15.0 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition; column: Waters Xbridge BEH C18 100×30 mm×10 μm; mobile phase: [water (FA)-ACN];B %: 15%-45%, 8 min) and prep-HPLC (FA condition; column: Phenomenex Luna C18 100× 30 mm× 5 μm; mobile phase: [water (FA)-ACN];B %: 15%-45%, 8 min) to give 2-[4-[2-(dimethylamino)ethoxy]anilino]-8-(3-hydroxycyclobutyl)-6-(5-methyl-4-prop-2-enoyl-2,3-dihydroquinoxalin-1-yl)pyrido[2,3-d]pyrimidin-7-one (26.3 mg, 40.7 μmol, 22.0% yield, 99.1% purity, FA) as a yellow solid.

The residue was separated by SFC (column: DAICEL CHIRALPAK IG (250 mm× 50 mm, 10 μm); mobile phase: [CO₂-IPA (0.1% NH₃H₂O)]; 50% B isocratic elution mode) to give 2-[4-[2-(dimethylamino)ethoxy]anilino]-8-(3-hydroxycyclobutyl)-6-(5-methyl-4-prop-2-enoyl-2,3-dihydroquinoxalin-1-yl)pyrido[2,3-d]pyrimidin-7-one (20.1 mg, 33.7 μmol, 50·2% yield, 100% purity) (Rt=2.025 min, 20.1 mg) and 2-[4-[2-(dimethylamino)ethoxy]anilino]-8-(3-hydroxycyclobutyl)-6-(5-methyl-4-prop-2-enoyl-2,3-dihydroquinoxalin-1-yl)pyrido[2,3-d]pyrimidin-7-one (4.1 mg, 6.9 μmol, 10.3% yield, 100% purity) (Rt=2.820 min, 4.1 mg) both as yellow

### Procedure for Preparation of Compound 047

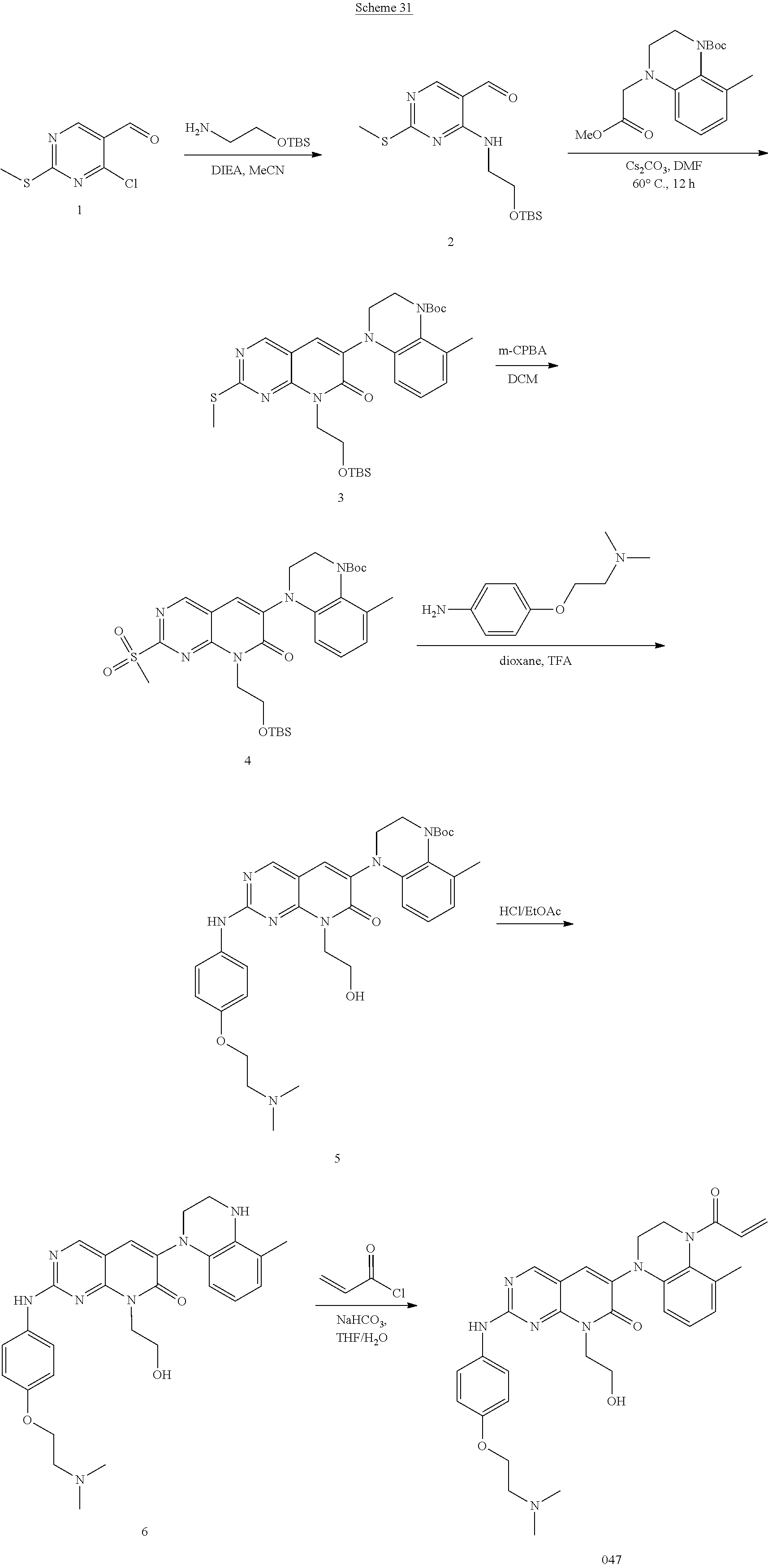

To a solution of 4-chloro-2-methylsulfanyl-pyrimidine-5-carbaldehyde (1 g, 5.3 mmol, 1 eq) in MeCN (10 mL) was added DIEA (2.1 g, 15.9 mmol, 2.8 mL, 3 eq) and 2-[tert-butyl(dimethyl)silyl]oxyethanamine (929.5 mg, 5.3 mmol, 1 eq). The mixture was stirred at 40° C. for 12 hr. LC-MS showed desired compound was detected. The reaction mixture was partitioned between H₂O (30 mL) and ethyl acetate (50 mL× 3). The organic phase was separated, washed with brine (20 mL× 2), dried over Na₂SO₄, filtered and concentrated under reduced pressure to give a residue. The residue was purified by flash silica gel chromatography (20 g Silica Flash Column, Eluent of 0˜4% Ethyl acetate/Petroleum ether gradient @ 100 mL/min) to give 4-[2-[tert-butyl(dimethyl)silyl]oxyethylamino]-2-methylsulfanyl-pyrimidine-5-carbaldehyde (1.7 g) as a yellow oil.

To a solution of 4-[2-[tert-butyl(dimethyl)silyl]oxyethylamino]-2-methylsulfanyl-pyrimidine-5-carbaldehyde (1.6 g, 4.9 mmol, 1.0 eq) in DMF (30 mL) was added Cs₂CO₃ (4.8 g, 14.7 mmol, 3 eq) and tert-butyl 4-(2-ethoxy-2-oxo-ethyl)-8-methyl-2,3-dihydroquinoxaline-1-carboxylate (1.6 g, 4.9 mmol, 1 eq). The mixture was stirred at 60° C. for 12 hr. LC-MS showed desired compound was detected. The reaction mixture was partitioned between H₂O (100 mL) and ethyl acetate (100 mL× 3). The organic phase was separated, washed with brine (100 ml× 2), dried over Na₂SO₄, filtered and concentrated under reduced pressure to give a residue. The residue was purified by flash silica gel chromatography (20 g Silica Flash Column, Eluent of 0˜10% Ethyl acetate/Petroleum ether gradient @ 100 mL/min) to give tert-butyl 4-[8-[2-[tert-butyl(dimethyl)silyl]oxyethyl]-2-methylsulfanyl-7-oxo-pyrido[2,3-d]pyrimidin-6-yl]-8-methyl-2,3-dihydroquinoxaline-1-carboxylate (2.6 g, 4.4 mmol, 89.0% yield) as a yellow oil.

To a solution of tert-butyl 4-[8-[2-[tert-butyl(dimethyl)silyl]oxyethyl]-2-methylsulfanyl-7-oxo-pyrido[2,3-d]pyrimidin-6-yl]-8-methyl-2,3-dihydroquinoxaline-1-carboxylate (2.6 g, 4.4 mmol, 1 eq) in DCM (30 mL) was added m-CPBA (1.3 g, 6.5 mmol, 85% purity, 1.5 eq) at 0° C. The mixture was stirred at 25° C. for 2 hr. LC-MS showed desired compound was detected. The reaction mixture was partitioned between Na₂SO₃ (100 mL) and dichloromethane (100×3 mL). The organic phase was separated, washed with NaHCO₃ (100 mL× 3), dried over Na₂SO₄, filtered and concentrated under reduced pressure to give tert-butyl 4-[8-[2-[tert-butyl(dimethyl)silyl]oxyethyl]-2-methylsulfonyl-7-oxo-pyrido[2,3-d]pyrimidin-6-yl]-8-methyl-2,3-dihydroquinoxaline-1-carboxylate (2.6 g, crude) as a yellow solid.

To a solution of tert-butyl 4-[8-[2-[tert-butyl(dimethyl)silyl]oxyethyl]-2-methylsulfonyl-7-oxo-pyrido[2,3-d]pyrimidin-6-yl]-8-methyl-2,3-dihydroquinoxaline-1-carboxylate (200 mg, 317.5 μmol, 1 eq) in dioxane (2 mL) was added TFA (72.4 mg, 635.1 μmol, 47·2 μL, 2 eq) and 4-[2-(dimethylamino)ethoxy]aniline (114.5 mg, 635.1 μmol, 2 eq). The mixture was stirred at 120° C. for 12 hr (batch×4). LC-MS showed desired compound was detected. The residue was purified by prep-HPLC (TFA condition) column: Phenomenex luna C18 100×40 mm×5 μm;mobile phase: [water (TFA)-ACN];B %: 35%-65%, 8 min to give tert-butyl 4-[8-[2-[tert-butyl(dimethyl)silyl]oxyethyl]-2-[4-[2-(dimethylamino)ethoxy]anilino]-7-oxo-pyrido[2,3-d]pyrimidin-6-yl]-8-methyl-2,3-dihydroquinoxaline-1-carboxylate (360 mg, 493·2 μmol, 38.8% yield) as a yellow solid.

A mixture of tert-butyl 4-[8-[2-[tert-butyl(dimethyl)silyl]oxyethyl]-2-[4-[2-(dimethylamino)ethoxy]anilino]-7-oxo-pyrido[2,3-d]pyrimidin-6-yl]-8-methyl-2,3-dihydroquinoxaline-1-carboxylate (90 mg, 123.3 μmol, 1 eq) in HCl/EtOAc (1 mL, 4 M) was stirred at 25° C. for 2 hr. LC-MS showed desired compound was detected. The reaction mixture was concentrated under reduced pressure to remove solvent to give 2-[4-[2-(dimethylamino)ethoxy]anilino]-8-(2-hydroxyethyl)-6-(5-methyl-3,4-dihydro-2H-quinoxalin-1-yl)pyrido[2,3-d]pyrimidin-7-one (90 mg, crude) as a yellow solid.

To a solution of 2-[4-[2-(dimethylamino)ethoxy]anilino]-8-(2-hydroxyethyl)-6-(5-methyl-3,4-dihydro-2H-quinoxalin-1-yl)pyrido[2,3-d]pyrimidin-7-one (90 mg, 163.0 μmol, 1 eq, HCl) in H₂O (0.5 mL) and THF (0.5 mL) was added NaHCO₃ (54.8 mg, 652.1 μmol, 25.4 μL, 4 eq) and prop-2-enoyl chloride (14.8 mg, 163.0 μmol, 13.3 μL, 1 eq) at 0° C. The mixture was stirred at 0° C. for 2 hr. LC-MS showed desired compound was detected. The reaction mixture was concentrated under reduced pressure to remove solvent. The residue was purified by prep-HPLC (TFA condition) column: Phenomenex luna C18 100×40 mm×5 μm; mobile phase: [water (TFA)-ACN];B %: 10%-40%, 8 min to give 2-[4-[2-(dimethylamino)ethoxy]anilino]-8-(2-hydroxyethyl)-6-(5-methyl-4-prop-2-enoyl-2,3-dihydroquinoxalin-1-yl)pyrido[2,3-d]pyrimidin-7-one (25.6 mg, 37.5 μmol, 23.0% yield, 100% purity, TFA) as a yellow solid.

### Procedure for Preparation of Compound 051

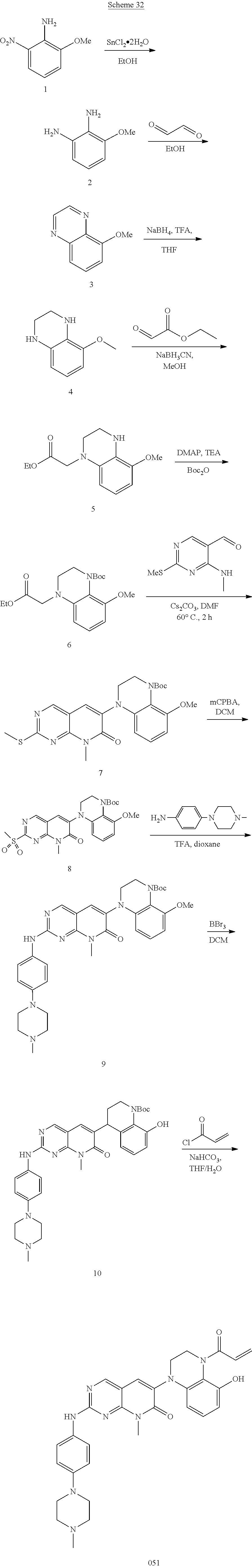

To a solution of 2-methoxy-6-nitro-aniline (10 g, 59.5 mmol, 10.0 mL, 1 eq) in EtOH (200 mL) was added SnCl₂·2H₂O (67.1 g, 297.4 mmol, 5 eq). The mixture was stirred at 75° C. for 3 hr (batch× 2). LC-MS showed desired compound was detected. The residue was diluted with 40% NaOH(200 mL) and extracted with ethyl acetate (300 mL× 3). The combined organic layers were washed with brine (300 mL× 1), dried over Na₂SO₄, filtered and concentrated under reduced pressure to give a residue. The residue was purified by flash silica gel chromatography (120 g Silica Flash Column, Eluent of 0˜26% Ethyl acetate/Petroleum ether gradient @ 120 mL/min) to give 3-methoxybenzene-1,2-diamine (14.4 g, 104.1 mmol, 87.5% yield) as a pink oil.

To a solution of 3-methoxybenzene-1,2-diamine (5 g, 36·2 mmol, 1 eq) in EtOH (200 mL) was added oxaldehyde (13.1 g, 90.5 mmol, 11.8 mL, 2.5 eq). The mixture was stirred at 80° C. for 2 hr (batch×2). LC-MS showed desired compound was detected. The reaction mixture was concentrated under reduced pressure to remove solvent to give 5-methoxyquinoxaline (11.5 g, crude) as a brown oil.

To a solution of 5-methoxyquinoxaline (5.8 g, 35.9 mmol, 1 eq) in THF (150 mL) was added NaBH₄ (6.8 g, 179.5 mmol, 5 eq) at 0° C., the mixture was stirred at 0° C. for 0.5 hr. Then the mixture was added TFA (10·2 g, 89.8 mmol, 6.7 mL, 2.5 eq) at 0° C., the mixture was stirred at 25° C. for 1 hr (batch× 2). LC-MS showed desired compound was detected. The reaction mixture was quenched by addition NH₄CI (400 mL) at 0° C., and then diluted with NH₄CI (200 mL) and extracted with ethyl acetate (400 mL× 3). The combined organic layers were washed with brine (500 mL× 1), dried over Na₂SO₄, filtered and concentrated under reduced pressure to give a residue. The residue was purified by flash silica gel chromatography (220 g Silica Flash Column, Eluent of 0˜25% Ethyl acetate/Petroleum ether gradient @ 120 mL/min) to give 5-methoxy-1,2,3,4-tetrahydroquinoxaline (6.5 g, 39.6 mmol, 55.1% yield) as a yellow oil.

To a solution of 5-methoxy-1,2,3,4-tetrahydroquinoxaline (2.5 g, 15·2 mmol, 1 eq) in

MeOH (25 mL) was added AcOH(91.4 mg, 1.5 mmol, 87·2 μL, 0.1 eq) and ethyl 2-oxoacetate (3.1 g, 15·2 mmol, 1 eq). The mixture was stirred at 60° C. for 1 hr. Then the mixture was added NaBH₃CN (2.9 g, 45.7 mmol, 3 eq), the mixture was stirred at 60° C. for 1 hr. LC-MS showed desired compound was detected. The residue was diluted with NaHCO₃ (80 mL) and extracted with ethyl acetate (80 mL×3). The combined organic layers were washed with brine (80 mL×1), dried over Na₂SO₄, filtered and concentrated under reduced pressure to give a residue. The residue was purified by flash silica gel chromatography (80 g Silica Flash Column, Eluent of 0˜12% Ethyl acetate/Petroleum ether gradient @ 120 mL/min) to give ethyl 2-(5-methoxy-3,4-dihydro-2H-quinoxalin-1-yl)acetate (2.8 g) as a yellow oil.

To a solution of ethyl 2-(5-methoxy-3,4-dihydro-2H-quinoxalin-1-yl)acetate (2.8 g, 11.3 mmol, 1 eq) in Boc₂O (40 mL) was added DMAP (138.1 mg, 1.1 mmol, 0.1 eq) and TEA (5.7 g, 56.5 mmol, 7.9 mL, 5 eq). The mixture was stirred at 80° C. for 12 hr. LC-MS showed desired compound was detected. The reaction mixture was concentrated under reduced pressure to remove solvent. The residue was purified by flash silica gel chromatography (40 g Silica Flash Column, Eluent of 0˜12% Ethyl acetate/Petroleum ether gradient @ 120 mL/min) to give tert-butyl 4-(2-ethoxy-2-oxo-ethyl)-8-methoxy-2,3-dihydroquinoxaline-1-carboxylate (3.7 g, 10.6 mmol, 93.4% yield) as a yellow oil.

To a solution of tert-butyl 4-(2-ethoxy-2-oxo-ethyl)-8-methoxy-2,3-dihydroquinoxaline-1-carboxylate (1.6 g, 4.6 mmol, 1 eq) in DMF (15 mL) was added 4-(methylamino)-2-methylsulfanyl-pyrimidine-5-carbaldehyde (836.7 mg, 4.6 mmol, 1 eq) and Cs₂CO₃ (3.0 g, 9.1 mmol, 2 eq). The mixture was stirred at 60° C. for 2 hr (batch×2). LC-MS showed desired compound was detected. The reaction mixture was concentrated under reduced pressure to remove solvent. The residue was diluted with H₂O (100 mL) and extracted with ethyl acetate (100 mL× 3). The combined organic layers were washed with brine (150 mL× 3), dried over Na₂SO₄, filtered and concentrated under reduced pressure to give a residue. The residue was purified by flash silica gel chromatography (80 g Silica Flash Column, Eluent of 0˜20% Ethyl acetate/Petroleum ether gradient @ 100 mL/min) to give tert-butyl 8-methoxy-4-(8-methyl-2-methylsulfanyl-7-oxo-pyrido[2,3-d]pyrimidin-6-yl)-2,3-dihydroquinoxaline-1-carboxylate (970 mg, 2.1 mmol, 22.6% yield) as a yellow solid.

To a solution of tert-butyl 8-methoxy-4-(8-methyl-2-methylsulfanyl-7-oxo-pyrido[2,3-d]pyrimidin-6-yl)-2,3-dihydroquinoxaline-1-carboxylate (770 mg, 1.6 mmol, 1 eq) in DCM (15 mL) was added mCPBA (499.4 mg, 2.5 mmol, 85% purity, 1.5 eq) at 0° C. The mixture was stirred at 25° C. for 2 hr. LC-MS showed desired compound was detected. The residue was diluted with Na₂SO₃ (30 mL) and extracted with dichloromethane (40 mL×4). The combined organic layers were washed with NaHCO₃ (30 mL×3), dried over Na₂SO₄, filtered and concentrated under reduced pressure to give tert-butyl 8-methoxy-4-(8-methyl-2-methylsulfonyl-7-oxo-pyrido[2,3-d]pyrimidin-6-yl)-2,3-dihydroquinoxaline-1-carboxylate (1.04g, crude) as a yellow solid.

To a solution of tert-butyl 8-methoxy-4-(8-methyl-2-methylsulfonyl-7-oxo-pyrido[2,3-d]pyrimidin-6-yl)-2,3-dihydroquinoxaline-1-carboxylate (200 mg, 398.8 μmol, 1 eq) in dioxane (2 mL) was added 4-(4-methylpiperazin-1-yl)aniline (91.5 mg, 478.5 μmol, 1·2 eq) and TFA (68·2 mg, 598.1 μmol, 44.4 μL, 1.5 eq). The mixture was stirred at 120° C. for 12 hr (batch×2). LC-MS showed desired compound was detected. The reaction mixture was concentrated under reduced pressure to remove solvent. The residue was purified by prep-HPLC (TFA condition column: Phenomenex Luna C18 75×30 mm×3 μm; mobile phase: [H₂O (0.1% TFA)-ACN]; gradient: 20%-55% B over 8.0 min) to give tert-butyl 8-methoxy-4-[8-methyl-2-[4-(4-methylpiperazin-1-yl)anilino]-7-oxo-pyrido[2,3-d]pyrimidin-6-yl]-2,3-dihydroquinoxaline-1-carboxylate (440 mg) as a yellow solid.

To a solution of tert-butyl 8-methoxy-4-[8-methyl-2-[4-(4-methylpiperazin-1-yl)anilino]-7-oxo-pyrido[2,3-d]pyrimidin-6-yl]-2,3-dihydroquinoxaline-1-carboxylate (50 mg, 81.6 μmol, 1 eq) in DCM (3 mL) was added BBr₃ (204.4 mg, 816.0 μmol, 78.6 μL, 10 eq) at 0° C. The mixture was stirred at 25° C. for 12 hr. LC-MS showed desired compound was detected. The reaction mixture was quenched by addition NH₃. H₂O (3 mL) at 0° C., and then diluted with H₂O (30 mL) and extracted with dichloromethane (40 mL×4), dried over Na₂SO₄, filtered and concentrated under reduced pressure to give 6-(5-hydroxy-3,4-dihydro-2H-quinoxalin-1-yl)-8-methyl-2-[4-(4-methylpiperazin-1-yl)anilino]pyrido[2,3-d]pyrimidin-7-one (42 mg, crude) as a yellow solid.

To a solution of 6-(5-hydroxy-3,4-dihydro-2H-quinoxalin-1-yl)-8-methyl-2-[4-(4-methylpiperazin-1-yl)anilino]pyrido[2,3-d]pyrimidin-7-one (22 mg, 44.1 μmol, 1 eq) in THF (1.5 mL) and H₂O (0.5 mL) was added NaHCO₃ (11.1 mg, 132.4 μmol, 5·2 μL, 3 eq) and prop-2-enoyl chloride (4.0 mg, 44.1 μmol, 3.6 μL, 1 eq) at 0° C. The mixture was stirred at 25° C. for 1 hr. LC-MS showed desired compound was detected. The reaction mixture was concentrated under reduced pressure to remove solvent. The residue was purified by prep-HPLC (TFA condition column: Phenomenex luna C18 100×40 mm×5 μm; mobile phase: [H₂O (0.1% TFA)-ACN]; gradient: 5%-35% B over 8.0 min) to give 6-(5-hydroxy-4-prop-2-enoyl-2,3-dihydroquinoxalin-1-yl)-8-methyl-2-[4-(4-methylpiperazin-1-yl)anilino]pyrido[2,3-d]pyrimidin-7-one (4.6 mg, 100% purity, TFA) as a yellow solid.

### Procedure for Preparation of Compound 054

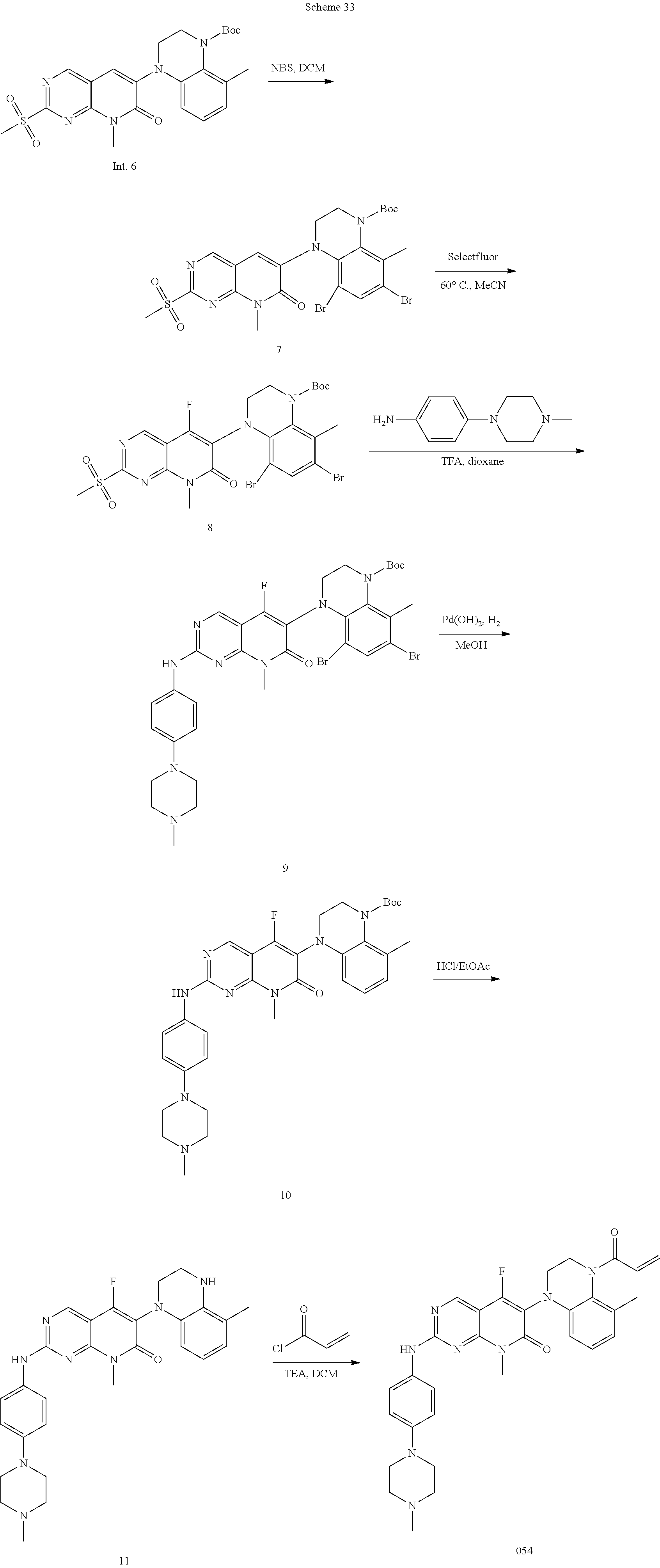

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (1 g, 2.1 mmol, 1 eq) in DCM (40 mL) was added NBS(916.4 mg, 5·2 mmol, 2.5 eq). The mixture was stirred at 25° C. for 12 hr (batch× 2). LC-MS showed desired compound was detected. The residue was diluted with NaHCO₃ (60 mL) and extracted with ethyl acetate (80 mL× 3). The combined organic layers were washed with brine (120 mL× 1), dried over Na₂SO₄, filtered and concentrated under reduced pressure to give a residue. The residue was purified by flash silica gel chromatography (40 g Silica Flash Column, Eluent of 0˜95% Ethyl acetate/Petroleum ether gradient @ 120 mL/min) to give tert-butyl 5,7-dibromo-8-methyl-4-(8-methyl-2-methylsulfonyl-7-oxo-pyrido[2,3-d]pyrimidin-6-yl)-2,3-dihydroquinoxaline-1-carboxylate (1.0 g, 1.6 mmol, 38.5% yield) as a yellow oil.

The reaction was set up in 3 batches in parallel: A mixture of tert-butyl 5,7-dibromo-8-methyl-4-(8-methyl-2-methylsulfonyl-7-oxo-pyrido[2,3-d]pyrimidin-6-yl)-2,3-dihydroquinoxaline-1-carboxylate (200 mg, 310.9 μmol, 1 eq), Selectfluor (143·2 mg, 404.1 μmol, 1.3 eq), 4A MOLECULAR SIEVE (300 mg) in MeCN (3 mL), and then the mixture was stirred at 60° C. for 12 hr under N₂ atmosphere. LC-MS showed desired compound was detected. The combined reaction mixture was filtered and concentrated under reduced pressure to give a residue. The residue was purified by prep-HPLC (FA condition column: Phenomenex Luna C18 100×30 mm× 5 μm; mobile phase: [H₂O (0·2% FA)-ACN]; gradient: 50%-80% B over 8.0 min) to give tert-butyl 5,7-dibromo-4-(5-fluoro-8-methyl-2-methylsulfonyl-7-oxo-pyrido[2,3-d]pyrimidin-6-yl)-8-methyl-2,3-dihydroquinoxaline-1-carboxylate (105 mg, 158.8 μmol, 17.0% yield) as a yellow solid.

To a solution of tert-butyl 5,7-dibromo-4-(5-fluoro-8-methyl-2-methylsulfonyl-7-oxo-pyrido[2,3-d]pyrimidin-6-yl)-8-methyl-2,3-dihydroquinoxaline-1-carboxylate (150 mg, 226.8 μmol, 1 eq) in dioxane (2 mL) was added 4-(4-methylpiperazin-1-yl)aniline (52.1 mg, 272·2 μmol, 1·2 eq) and TFA (38.8 mg, 340·2 μmol, 25.3 μL, 1.5 eq). The mixture was stirred at 120° C. for 12 hr (batch× 2). LC-MS showed desired compound was detected. The reaction mixture was concentrated under reduced pressure to remove solvent. The residue was purified by prep-HPLC (TFA condition column: Phenomenex luna C18 100×40 mm× 5 μm; mobile phase: [H₂O (0.1% TFA)-ACN]; gradient: 35%-65% B over 8.0 min) to give tert-butyl 5,7-dibromo-4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (160 mg) as a yellow solid.

To a solution of Pd(OH)₂ (30 mg, 20% purity) in MeOH (3 mL) was added tert-butyl 5,7-dibromo-4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (50 mg, 64.7 μmol, 1 eq) under N₂ atmosphere. The suspension was degassed and purged with H₂ for 3 times. The mixture was stirred under H₂ (15 psi) at 25° C. for 3 hr. LC-MS showed desired compound was detected. The reaction mixture filtered and concentrated under reduced pressure to give tert-butyl 4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (55 mg, crude) as a yellow oil.

A mixture of tert-butyl 4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (55 mg, 89.5 μmol, 1 eq) in HCl/EtOAc (0.8 mL, 4M) was stirred at 25° C. for 1 hr. LC-MS showed desired compound was detected. The reaction mixture was concentrated under reduced pressure to give 5-fluoro-8-methyl-6-(5-methyl-3,4-dihydro-2H-quinoxalin-1-yl)-2-[4-(4-methylpiperazin-1-yl)anilino]pyrido[2,3-d]pyrimidin-7-one (53 mg, crude, HCl) as a yellow solid.

To a solution of 5-fluoro-8-methyl-6-(5-methyl-3,4-dihydro-2H-quinoxalin-1-yl)-2-[4-(4-methylpiperazin-1-yl)anilino]pyrido[2,3-d]pyrimidin-7-one (53 mg, 96·2 μmol, 1 eq, HCl) in DCM (0.8 mL) was added TEA (29·2 mg, 288.5 μmol, 40·2 L, 3 eq) and prop-2-enoyl chloride (8.7 mg, 96·2 μmol, 7.8 μL, 1 eq) at 0° C. The mixture was stirred at 0° C. for 0.5 hr. LC-MS showed desired compound was detected. The reaction mixture was concentrated under reduced pressure to remove solvent. The residue was purified by prep-HPLC (TFA condition column: Phenomenex luna C18 100×40 mm× 5 μm; mobile phase: [H₂O (0.1% TFA)-ACN]; gradient: 15%-45% B over 8.0 min) to give 5-fluoro-8-methyl-2-[4-(4-methylpiperazin-1-yl)anilino]-6-(5-methyl-4-prop-2-enoyl-2,3-dihydroquinoxalin-1-yl)pyrido[2,3-d]pyrimidin-7-one (9.1 mg, 13.3 μmol, 13.8% yield, 99.9% purity, TFA) as a yellow solid.

### Procedure for Preparation of Compound 059

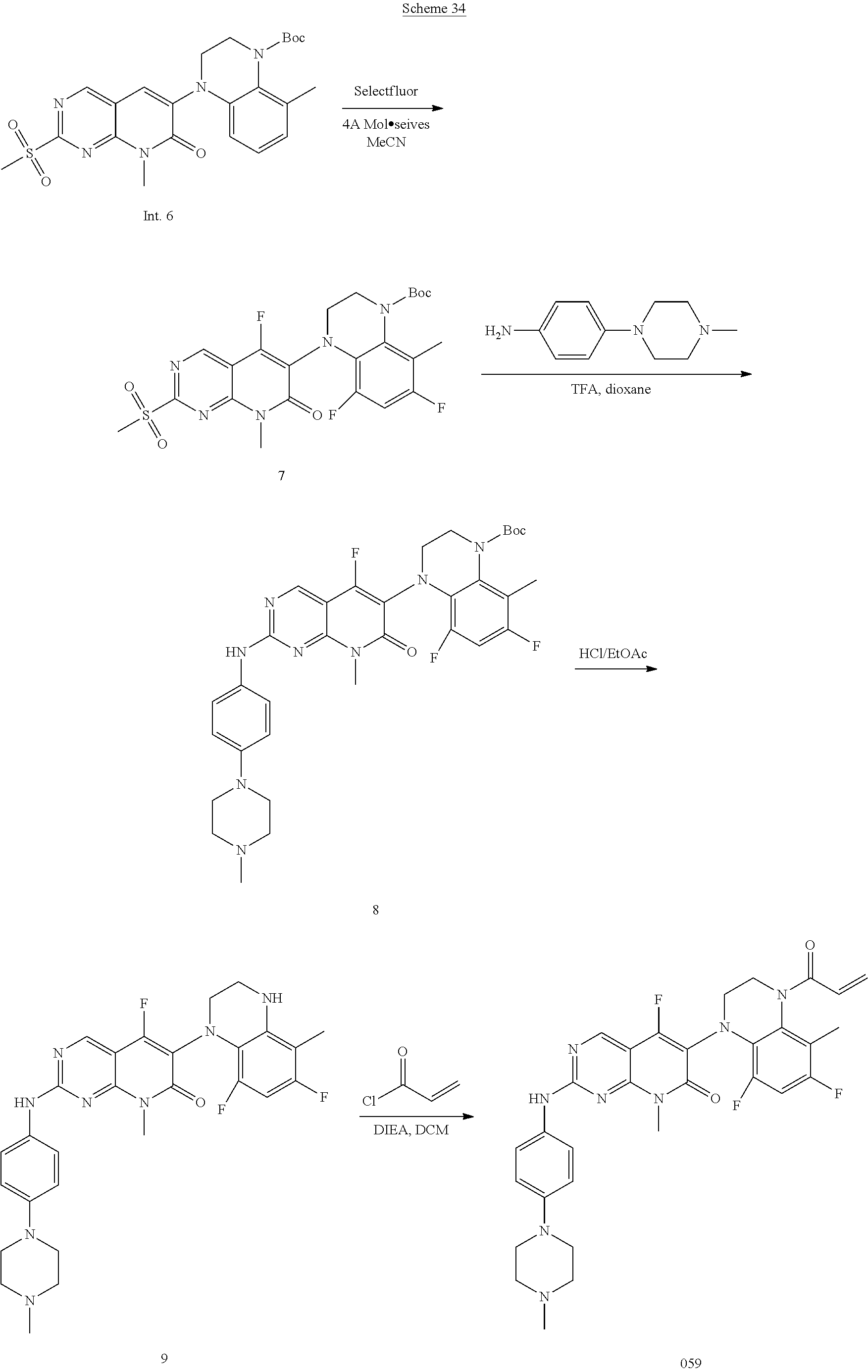

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (200 mg, 411.9 μmol, 1 eq) in MeCN (3 mL) was added 4A MOLECULAR SIEVE (400 mg) and 1-(chloromethyl)-4-fluoro-1,4-diazoniabicyclo[2·2.2]octane; ditetrafluoroborate (466.94 mg, 1.32 mmol, 3·2 eq). The mixture was stirred at 60° C. for 12 hr (batch×10). Nitrogen gas bubbled out during the addition of diazoatetate. LCMS showed consumption of reactant and formation of the desired product mass.

The reaction mixture was quenched by addition H₂O (100 mL) at 0° C., and extracted with ethyl acetate (100 mL× 3). The combined organic layers were washed with brine (250 ml× 1), dried over Na₂SO₄, filtered and concentrated under reduced pressure to give a residue. The residue was purified by flash silica gel chromatography (40 g Silica Flash Column, Eluent of 0˜100% Ethyl acetate/Petroleum ether gradient @ 120 mL/min). The residue was purified by prep-HPLC (TFA condition; column: Phenomenex Luna C18 100×30 mm× 5 μm; mobile phase: [H₂O (0·2% FA)-ACN]; gradient: 30%-60% B over 8.0 min) to yield compound tert-butyl 5,7-difluoro-4-(5-fluoro-8-methyl-2-methylsulfonyl-7-oxo-pyrido[2,3-d]pyrimidin-6-yl)-8-methyl-2,3-dihydroquinoxaline-1-carboxylate (100 mg, 185.3 umol, 4.5% yield) obtained as a yellow solid.

To a solution of tert-butyl 5,7-difluoro-4-(5-fluoro-8-methyl-2-methylsulfinyl-7-oxo-pyrido[2,3-d]pyrimidin-6-yl)-8-methyl-2,3-dihydroquinoxaline-1-carboxylate (40 mg, 76.4 μmol, 1 eq) in dioxane (1 mL) was added TFA (13.1 mg, 114.6 μmol, 8.5 μL, 1.5 eq) and 4-(4-methylpiperazin-1-yl)aniline (17.5 mg, 91.7 μmol, 1·2 eq). The mixture was stirred at 120° C. for 12 hr. The reaction was monitored by LCMS which showed the reactant was consumed completely and the desired mass peak was detected. The reaction mixture was concentrated under reduced pressure to give a residue. The reaction mixture was concentrated under reduced pressure to remove solvent. The residue was purified by prep-HPLC (TFA condition; column: Phenomenex luna C18 100×40 mm×5 μm; mobile phase: [H₂O (0.1% TFA)-ACN]; gradient: 35%-65% B over 8.0 min) to yield compound tert-butyl 5,7-difluoro-4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (50 mg, 76.8 μmol, 50.3% yield) obtained as a yellow solid.

A mixture of tert-butyl 5,7-difluoro-4-[5-fluoro-8-methyl-2-[4-(4-methylpiperazin-1-yl)anilino]-7-oxo-pyrido[2,3-d]pyrimidin-6-yl]-8-methyl-2,3-dihydroquinoxaline-1-carboxylate (25 mg, 38.4 μmol, 1 eq) in HCl/EtOAc (1 mL, 4M) was stirred at 25° C. for1 hr. The reaction was monitored by LCMS which showed complete consumption of reactant and detection of the desired mass peak. The reaction mixture was concentrated under reduced pressure to give the crude product 6-(6,8-difluoro-5-methyl-3,4-dihydro-2H-quinoxalin-1-yl)-5-fluoro-8-methyl-2-[4-(4-methylpiperazin-1-yl)anilino]pyrido[2,3-d]pyrimidin-7-one (25 mg, crude) obtained as yellow solid used into the next step without further purification.

To a solution of 6-(6,8-difluoro-5-methyl-3,4-dihydro-2H-quinoxalin-1-yl)-5-fluoro-8-methyl-2-[4-(4-methylpiperazin-1-yl)anilino]pyrido[2,3-d]pyrimidin-7-one (20 mg, 36.3 μmol, 1 eq) in DCM (1 mL) was added DIEA (14.1 mg, 109.0 μmol, 19.0 μL, 3 eq) and prop-2-enoyl chloride (3.3 mg, 36.3 μmol, 2.9 μL, 1 eq) at 0° C. The mixture was stirred at 0° c. for1 hr. The reaction was monitored by LCMS which showed complete consumption of reactant and detection of the desired mass peak. The reaction mixture was concentrated under reduced pressure to remove solvent. The residue was diluted with dimethyl formamide (3 mL). The residue was purified by prep-HPLC (TFA condition; column: Phenomenex luna C18 100×40 mm× 5 μm; mobile phase: [H₂O (0.1% TFA)-ACN];gradient: 15%-45% B over 8.0 min) to yield compound 6-(6,8-difluoro-5-methyl-4-prop-2-enoyl-2,3-dihydroquinoxalin-1-yl)-5-fluoro-8-methyl-2-[4-(4-methylpiperazin-1-yl)anilino]pyrido[2,3-d]pyrimidin-7-one (4 mg, 6.5 μmol, 17.8% yield, 97.744% purity) obtained as a white solid. Analytic data 1H NMR and QC were consistent with the desired product.

### Procedure for Preparation of Compound 064

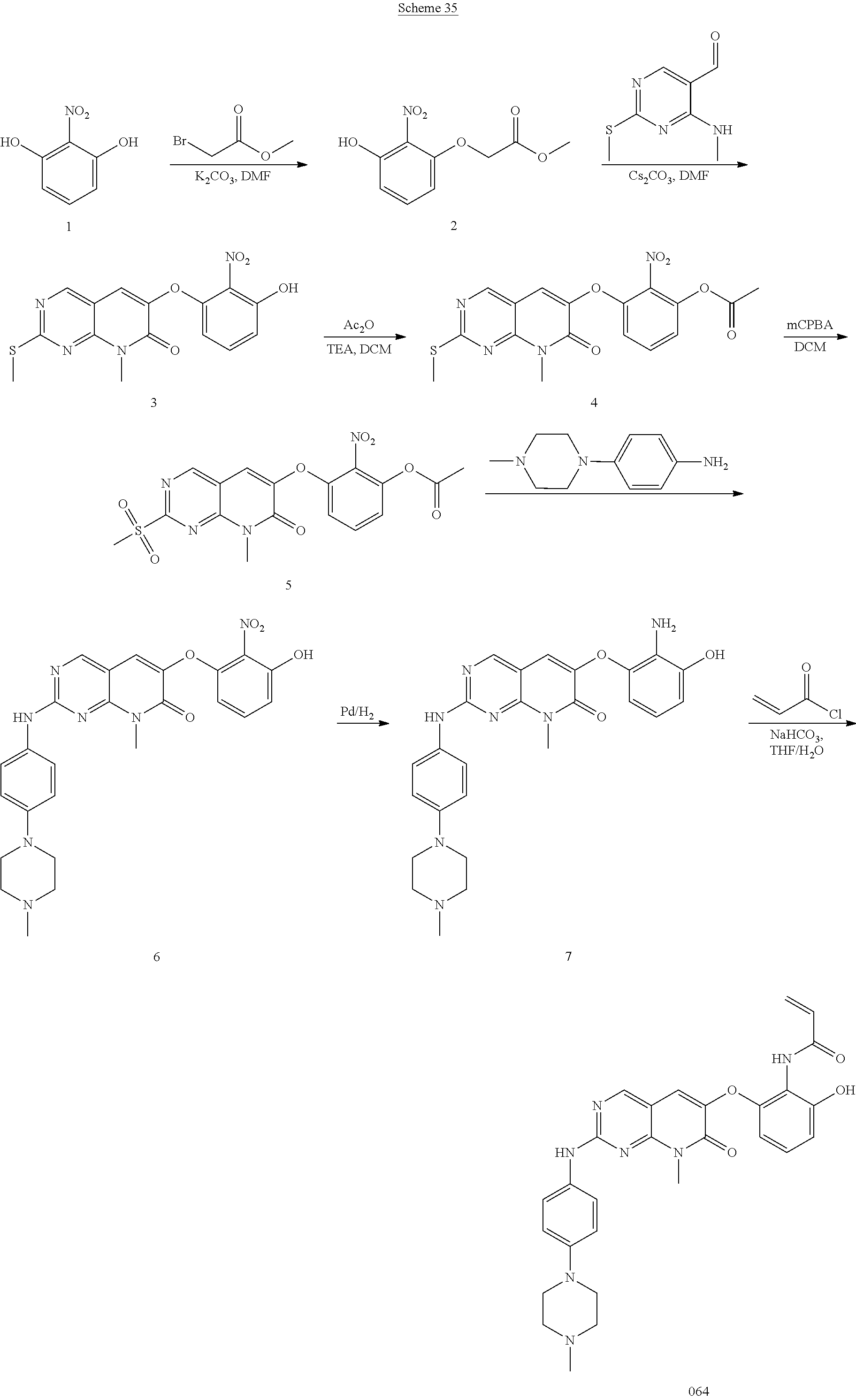

To a solution of 2-nitrobenzene-1,3-diol (5 g, 32·2 mmol, 1 eq) in DMF (50 mL) was added K₂CO₃ (4.5 g, 32·2 mmol, 1 eq) and methyl 2-bromoacetate (4.9 g, 32·2 mmol, 3.1 mL, 1 eq). The mixture was stirred at 20° C. for 2 hr (batch×4). LC-MS showed desired compound was detected. The reaction mixture was partitioned between NaHCO₃ (600 mL) and ethyl acetate (600 mL× 3). The organic phase was separated, washed with brine (500 mL× 2), dried over Na₂SO₄, filtered and concentrated under reduced pressure to give a residue. The residue was purified by flash silica gel chromatography (80 g Silica Flash Column, Eluent of 0˜10% Ethyl acetate/Petroleum ether gradient @ 100 mL/min) to give methyl 2-(3-hydroxy-2-nitro-phenoxy)acetate (5·2 g, 22.9 mmol, 17.8% yield) as a yellow solid.

To a solution of methyl 2-(3-hydroxy-2-nitro-phenoxy)acetate (5·2 g, 22.9 mmol, 1 eq) in DMF (52 mL) was added Cs₂CO₃ (22.4 g, 68.7 mmol, 3 eq) and 4-(methylamino)-2-methylsulfanyl-pyrimidine-5-carbaldehyde (4·2 g, 22.9 mmol, 1 eq). The mixture was stirred at 80° C. for 12 hr. LC-MS showed desired compound was detected. The reaction mixture was partitioned between H₂O (300 mL) and ethyl acetate (300 mL× 3). The organic phase was separated, washed with brine (200 mL× 2), dried over Na₂SO₄, filtered and concentrated under reduced pressure to give a residue. The crude product was triturated with ethyl acetate and water at 20° C. for 15 min. The reaction mixture was filtered and the filter cake dried to give 6-(3-hydroxy-2-nitro-phenoxy)-8-methyl-2-methylsulfanyl-pyrido[2,3-d]pyrimidin-7-one (5.9 g, 16.4 mmol, 71.5% yield) as a yellow solid.

To a solution of 6-(3-hydroxy-2-nitro-phenoxy)-8-methyl-2-methylsulfanyl-pyrido[2,3-d]pyrimidin-7-one (2 g, 5.55 mmol, 1 eq) in DCM (20 mL) was added TEA (2.81 g, 27.75 mmol, 3.86 mL, 5 eq) and acetyl acetate (1.42 g, 13.88 mmol, 1.30 mL, 2.5 eq) at 0° C. The mixture was stirred at 20° C. for 12 hr. LC-MS showed desired compound was detected. The reaction mixture was partitioned between NaHCO₃300 mL and ethyl acetate (500 mL× 3). The organic phase was separated, washed with brine (300 mL× 2), dried over Na₂SO₄, filtered and concentrated under reduced pressure to give a residue to give [3-(8-methyl-2-methylsulfanyl-7-oxo-pyrido[2,3-d]pyrimidin-6-yl)oxy-2-nitro-phenyl]acetate (1·2 g, crude) as a yellow solid.

To a solution of [3-(8-methyl-2-methylsulfanyl-7-oxo-pyrido[2,3-d]pyrimidin-6-yl)oxy-2-nitro-phenyl]acetate (1·2 g, 3.0 mmol, 1 eq) in DCM (12 mL) was added mCPBA (1·2 g, 6.0 mmol, 85% purity, 2 eq) at 0° C. The mixture was stirred at 20° C. for 12 hr. LC-MS showed desired compound was detected. The reaction mixture was partitioned between Na₂SO_{3 200} mL and dichloromethane (300× 3 mL). The organic phase was separated, washed with NaHCO₃ (300 mL× 3), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 100 mL/min) to give [3-(8-methyl-2-methylsulfonyl-7-oxo-pyrido[2,3-d]pyrimidin-6-yl)oxy-2-nitro-phenyl]acetate (800 mg, 1.8 mmol, 61.8% yield) as a yellow solid.

To a solution of [3-(8-methyl-2-methylsulfonyl-7-oxo-pyrido[2,3-d]pyrimidin-6-yl)oxy-2-nitro-phenyl]acetate (150 mg, 345.3 μmol, 1 eq) in DMSO (2 mL) was added 4-(4-methylpiperazin-1-yl)aniline (99.1 mg, 518.0 μmol, 1.5 eq), DIEA (133.9 mg, 1.0 mmol, 180.5 μL, 3 eq) and CsF (52.5 mg, 345.3 μmol, 12.8 μL, 1 eq). The mixture was stirred at 80° C. for 12 hr (batch× 3). LC-MS showed desired compound was detected. The reaction mixture was concentrated under reduced pressure to remove solvent. The residue was purified by prep-HPLC (TFA condition) column: Phenomenex luna C18 100×40 mm×5 μm; mobile phase: [H₂O (0.1% TFA)-ACN]; gradient: 15%-45% B over 8.0 min to give 6-(3-hydroxy-2-nitro-phenoxy)-8-methyl-2-[4-(4-methylpiperazin-1-yl)anilino]pyrido[2,3-d]pyrimidin-7-one (40 mg, 79.4 μmol, 7.7% yield) as a yellow solid.

To a solution of Pd/C (40 mg, 10% purity) in MeOH (1 mL) was added 6-(3-hydroxy-2-nitro-phenoxy)-8-methyl-2-[4-(4-methylpiperazin-1-yl)anilino]pyrido[2,3-d]pyrimidin-7-one (40 mg, 79.4 μmol, 1 eq) under N₂ atmosphere. The mixture was stirred under H₂ (15 Psi) at 20° C. for 3 hr. LC-MS showed desired compound was detected. The reaction mixture was filtered and the filtrate dried in vacuum to a residue to give 6-(2-amino-3-hydroxy-phenoxy)-8-methyl-2-[4-(4-methylpiperazin-1-yl)anilino]pyrido[2,3-d]pyrimidin-7-one (40 mg, crude) as a yellow solid.

To a solution of 6-(2-amino-3-hydroxy-phenoxy)-8-methyl-2-[4-(4-methylpiperazin-1-yl)anilino]pyrido[2,3-d]pyrimidin-7-one (40 mg, 84.5 μmol, 1 eq) in THF (0.3 mL) and H₂O (0.1 mL) was added NaHCO₃ (21.3 mg, 253.4 μmol, 9.9 μL, 3 eq) and prop-2-enoyl chloride (7.7 mg, 84.5 μmol, 6.9 μL, 1 eq) at 0° C. The mixture was stirred at 20° C. for 30 min. LC-MS showed desired compound was detected. The reaction mixture was concentrated under reduced pressure to remove solvent. The residue was purified by prep-HPLC (TFA condition) column: Phenomenex luna C18 100×40 mm×5 μm; mobile phase: [H₂O (0.1% TFA)-ACN];gradient: 10%-45% B over 8.0 min to give N-[2-hydroxy-6-[8-methyl-2-[4-(4-methylpiperazin-1-yl)anilino]-7-oxo-pyrido[2,3-d]pyrimidin-6-yl]oxy-phenyl]prop-2-enamide (6.03 mg, 11.43 μmol, 13.53% yield, 100% purity) as a yellow solid.

### Procedure for Preparation of Compound 056 and Compound 057

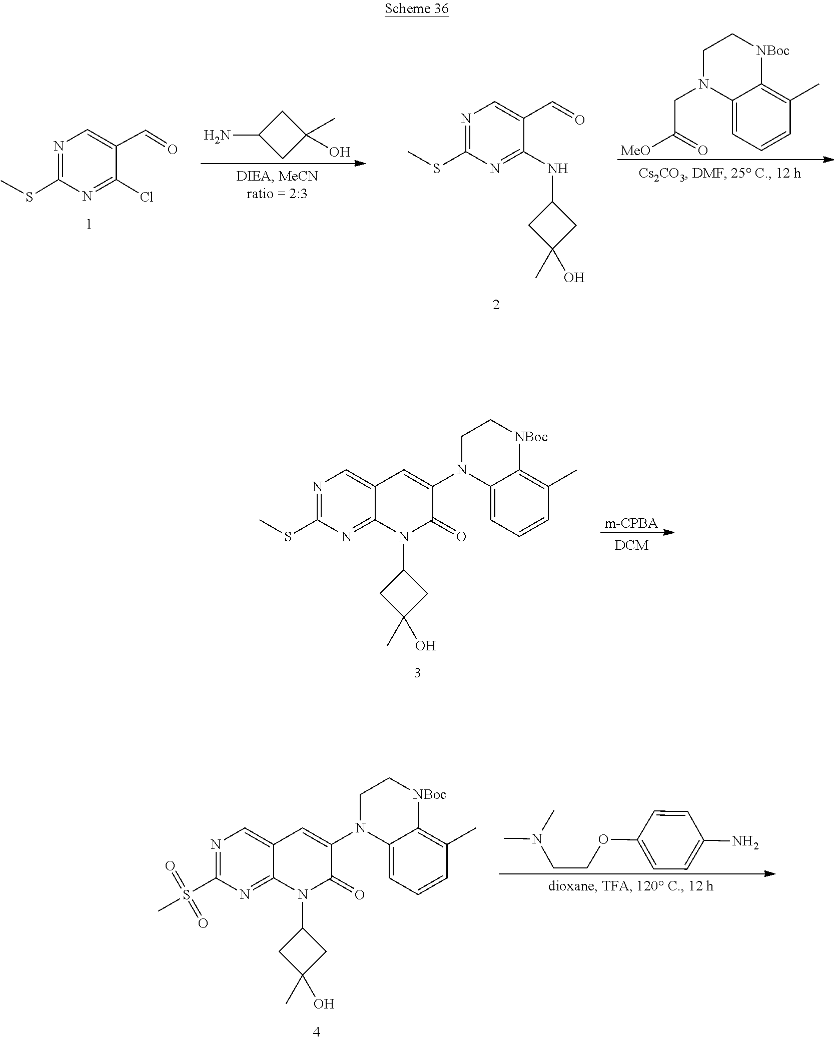

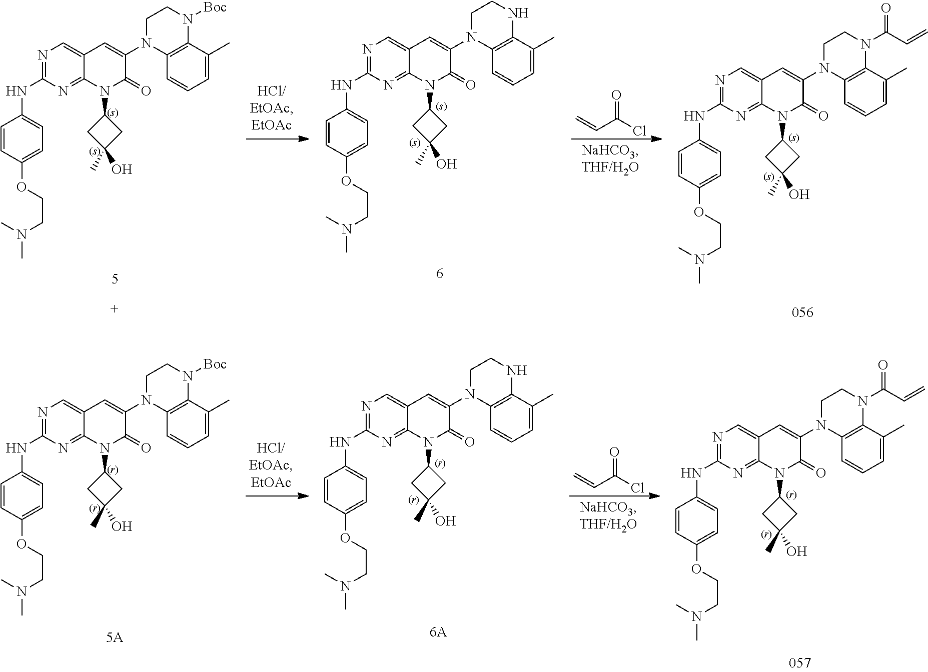

To a solution of 4-chloro-2-methylsulfanyl-pyrimidine-5-carbaldehyde (900 mg, 4.7 mmol, 1 eq) in MeCN (30 mL) was added DIEA (1.8 g, 14.3 mmol, 2.5 mL, 3 eq) and 3-amino-1-methyl-cyclobutanol (482.6 mg, 4.7 mmol, 1 eq). The mixture was stirred at 20° C. for 12 hr. LC-MS showed the desired compound was detected. The reaction mixture was partitioned between H₂O (100 mL) and ethyl acetate (100 mL× 3). The organic phase was separated, washed with brine (60 mL× 3), dried over Na₂SO₄, filtered and concentrated under reduced pressure to give a residue. The residue was purified by flash silica gel chromatography (40 g Silica Flash Column, Eluent of 0˜50% Ethyl acetate/Petroleum ether gradient @ 120 mL/min) to give 4-[(3-hydroxy-3-methyl-cyclobutyl)amino]-2-methylsulfanyl-pyrimidine-5-carbaldehyde (1.0 g, 4·2 mmol, 43.8% yield) as a brown oil.

To a solution of 4-[(3-hydroxy-3-methyl-cyclobutyl)amino]-2-methylsulfanyl-pyrimidine-5-carbaldehyde (1.0 g, 4·2 mmol, 1 eq) in DMF (20 mL) was added Cs₂CO₃ (4.1 g, 12.5 mmol, 3 eq) and tert-butyl 4-(2-ethoxy-2-oxo-ethyl)-8-methyl-2,3-dihydroquinoxaline-1-carboxylate (1.4 g, 4·2 mmol, 1 eq). The mixture was stirred at 25° C. for 12 hr. LC-MS showed the desired compound was detected. The reaction mixture was partitioned between H₂O (100 mL) and ethyl acetate (100 mL× 3). The organic phase was separated, washed with brine (50 mL× 3), dried over Na₂SO₄, filtered and concentrated under reduced pressure to give a residue. The residue was purified by flash silica gel chromatography (25 g Silica Flash Column, Eluent of 0˜30% Ethyl acetate/Petroleum ether gradient @ 120 mL/min) to give tert-butyl 4-[8-(3-hydroxy-3-methyl-cyclobutyl)-2-methylsulfanyl-7-oxo-pyrido[2,3-d]pyrimidin-6-yl]-8-methyl-2,3-dihydroquinoxaline-1-carboxylate (1.3 g, 2.45 mmol, 59.3% yield) as a light yellow solid.

To a solution of tert-butyl 4-[8-(3-hydroxy-3-methyl-cyclobutyl)-2-methylsulfanyl-7-oxo-pyrido[2,3-d]pyrimidin-6-yl]-8-methyl-2,3-dihydroquinoxaline-1-carboxylate (1.1 g, 2.1 mmol, 1 eq) in DCM (20 mL) was added m-CPBA (639.7 mg, 3.1 mmol, 85% purity, 1.5 eq) at 0° C. The mixture was stirred at 25° C. for 2 hr. LC-MS showed the desired compound was detected. The reaction mixture was partitioned between Na₂SO₃ (50 mL) and dichloromethane (50×3 mL). The organic phase was separated, washed with NaHCO₃ (50 mL× 3), dried over Na₂SO₄, filtered and concentrated under reduced pressure to give tert-butyl 4-[8-(3-hydroxy-3-methyl-cyclobutyl)-2-methylsulfonyl-7-oxo-pyrido[2,3-d]pyrimidin-6-yl]-8-methyl-2,3-dihydroquinoxaline-1-carboxylate (1.1 g, crude) as a light yellow solid.

To a solution of tert-butyl 4-[8-(3-hydroxy-3-methyl-cyclobutyl)-2-methylsulfonyl-7-oxo-pyrido[2,3-d]pyrimidin-6-yl]-8-methyl-2,3-dihydroquinoxaline-1-carboxylate (600 mg, 359.9 μmol, 1 eq) in dioxane (6 mL) was added TFA (246·2 mg, 719.8 μmol, 160.4 μL, 2 eq) and 4-[2-(dimethylamino)ethoxy]aniline (390 mg, 719.8 μmol, 2 eq). The mixture was stirred at 120° C. for 12 hr. LC-MS showed the desired compound was detected. The reaction mixture was concentrated under reduced pressure to give a residue. The residue was purified by prep-HPLC (TFA condition, column: Phenomenex luna C18 100×40 mm×5 μm; mobile phase: [H₂O (0.1% TFA)-ACN]; gradient: 25%-55% B over 8.0 min) to give tert-butyl 4-(2-((4-(2-(dimethylamino)ethoxy)phenyl)amino)-8-((1s,3s)-3-hydroxy-3-methylcyclobutyl)-7-oxo-7,8-dihydropyrido[2,3-d]pyrimidin-6-yl)-8-methyl-3,4-dihydroquinoxaline-1 (2H)-carboxylate (160 mg, 262.4 μmol, 24.3% yield) as a light yellow solid and tert-butyl 4-(2-((4-(2-(dimethylamino)ethoxy)phenyl)amino)-8-((1r,3r)-3-hydroxy-3-methylcyclobutyl)-7-oxo-7,8-dihydropyrido[2,3-d]pyrimidin-6-yl)-8-methyl-3,4-dihydroquinoxaline-1 (2H)-carboxylate (190.0 mg, 289.7 μmol, 1 eq) as a light yellow solid.

To a solution of tert-butyl 4-(2-((4-(2-(dimethylamino)ethoxy)phenyl)amino)-8-((1s,3s)-3-hydroxy-3-methylcyclobutyl)-7-oxo-7,8-dihydropyrido[2,3-d]pyrimidin-6-yl)-8-methyl-3,4-dihydroquinoxaline-1 (2H)-carboxylate (140.0 mg, 213.4 μmol, 1 eq) in EtOAc (2 mL) was added HCl/EtOAc (2 mL, 4 M). The mixture was stirred at 20° C. for 1 hr. LC-MS showed the desired compound was detected. The reaction mixture was concentrated under reduced pressure to give 2-[4-[2-(dimethylamino)ethoxy]anilino]-8-((1s,3s)-3-hydroxy-3-methyl-cyclobutyl)-6-(5-methyl-3,4-dihydro-2H-quinoxalin-1-yl)pyrido[2,3-d]pyrimidin-7-one (140 mg, crude, HCl) as a yellow solid.

To a solution of 2-[4-[2-(dimethylamino)ethoxy]anilino]-8-((1s,3s)-3-hydroxy-3-methyl-cyclobutyl)-6-(5-methyl-3,4-dihydro-2H-quinoxalin-1-yl)pyrido[2,3-d]pyrimidin-7-one (140.0 mg, 251.9 μmol, 1 eq) in THF (3 mL) and H₂O (1 mL) was added NaHCO₃ (127.0 mg, 1.5 mmol, 58.8 μL, 6 eq) and prop-2-enoyl chloride (27.3 mg, 302.3 μmol, 24.5 L, 1·2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex luna C18 100×40 mm×5 μm; mobile phase: [H₂O (0.1% TFA)-ACN]; gradient: 15%-45% B over 8.0 min) to give 2-[4-[2-(dimethylamino)ethoxy]anilino]-8-((1s,3s)-3-hydroxy-3-methyl-cyclobutyl)-6-(5-methyl-4-prop-2-enoyl-2,3-dihydroquinoxalin-1-yl)pyrido[2,3-d]pyrimidin-7-one (86.5 mg, 118.0 μmol, 46.8% yield, 98.6% purity, TFA) as a white solid.

To a solution of tert-butyl 4-(2-((4-(2-(dimethylamino)ethoxy)phenyl)amino)-8-((1r,3r)-3-hydroxy-3-methylcyclobutyl)-7-oxo-7,8-dihydropyrido[2,3-d]pyrimidin-6-yl)-8-methyl-3,4-dihydroquinoxaline-1 (2H)-carboxylate (190.0 mg, 289.7 μmol, 1 eq) in EtOAc (1 mL) was added HCl/EtOAc (1 mL,4 M) at 0° C. The mixture was stirred at 0° C. for 2 hr. LC-MS showed desired compound was detected. The reaction mixture was concentrated under reduced pressure to remove solvent to give 2-[4-[2-(dimethylamino)ethoxy]anilino]-8-((1r,3r)-3-hydroxy-3-methyl-cyclobutyl)-6-(5-methyl-3,4-dihydro-2H-quinoxalin-1-yl)pyrido[2,3-d]pyrimidin-7-one (Compound 056) (190 mg, crude) as a yellow solid.

To a solution of 2-[4-[2-(dimethylamino)ethoxy]anilino]-8-((1r,3r)-3-hydroxy-3-methyl-cyclobutyl)-6-(5-methyl-3,4-dihydro-2H-quinoxalin-1-yl)pyrido[2,3-d]pyrimidin-7-one (190.0 mg, 341.9 μmol, 1 eq) in THF (1.5 mL) and H₂O (0.5 mL) was added NaHCO₃ (86·2 mg, 1.0 mmol, 39.9 μL, 3 eq) and prop-2-enoyl chloride (31.0 mg, 341.9 μmol, 27.8 μL, 1 eq) at 0° C. The mixture was stirred at 0° C. for 2 hr. LC-MS showed desired compound was detected. The reaction mixture was concentrated under reduced pressure to remove solvent. The residue was purified by prep-HPLC (TFA condition) column: Phenomenex Luna C18 75*30 mm*3 μm; mobile phase: [H₂O (0.1% TFA)-ACN]; gradient: 20%-50% B over 8.0 min to give 2-[4-[2-(dimethylamino)ethoxy]anilino]-8-((1r,3r)-3-hydroxy-3-methyl-cyclobutyl)-6-(5-methyl-4-prop-2-enoyl-2,3-dihydroquinoxalin-1-yl)pyrido[2,3-d]pyrimidin-7-one (Compound 057) (79.7 mg, 108.7 μmol, 31.8% yield, 98.7% purity, TFA) as a yellow solid.

### Procedure for Preparation of Compound 058

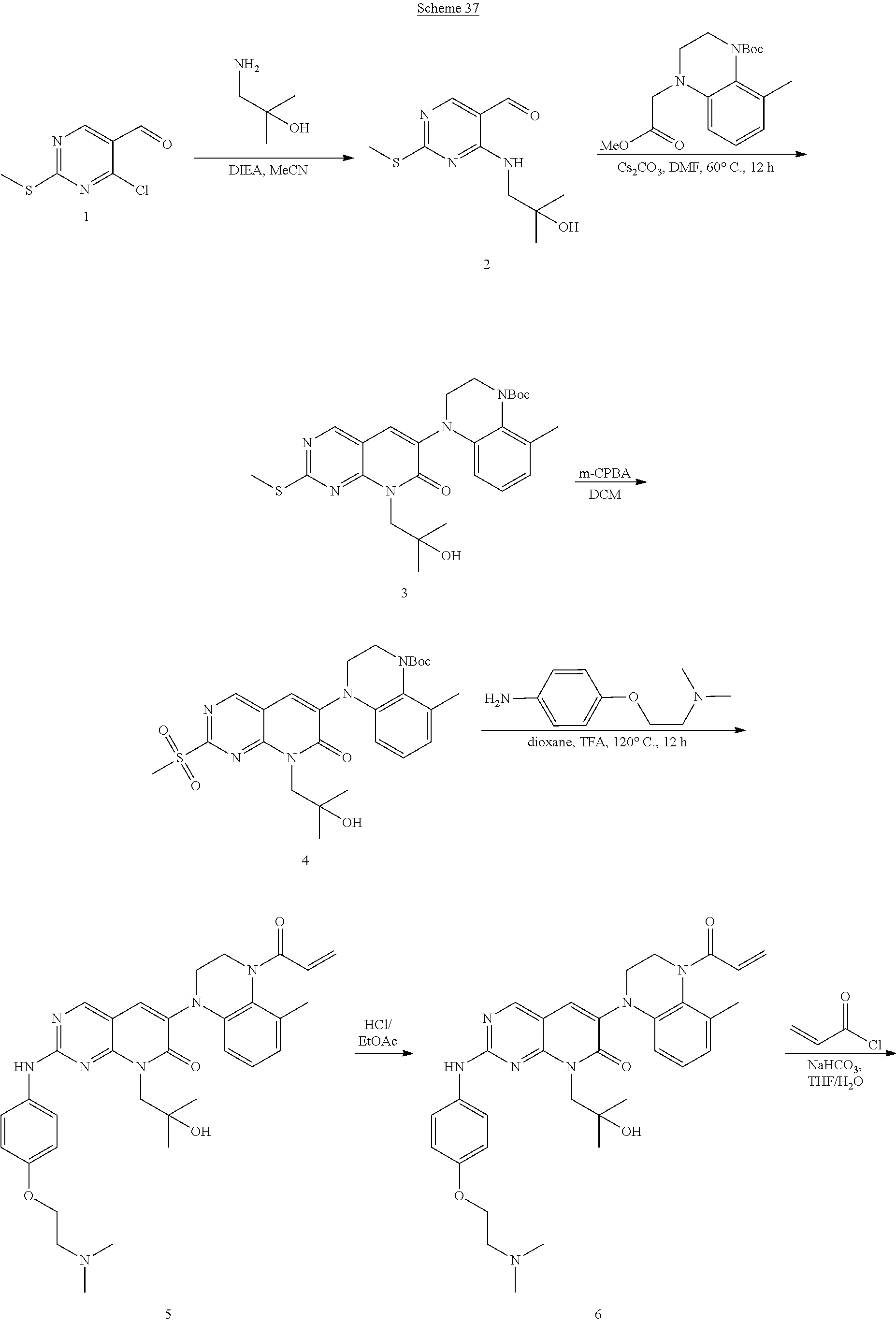

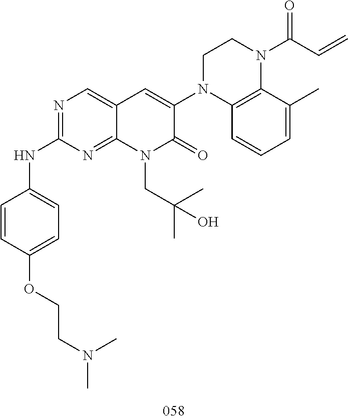 [SMILES: C=CC(=O)N1CCN(c2cc3cnc(Nc4ccc(OCCN(C)C)cc4)nc3n(CC(C)(C)O)c2=O)c2cccc(C)c21]

To a solution of 4-chloro-2-methylsulfanyl-pyrimidine-5-carbaldehyde (2.6 g, 13.8 mmol, 1 eq) in MeCN (60 mL) was added 1-amino-2-methyl-propan-2-ol (1·2 g, 13.8 mmol, 1 eq) and DIEA (5.34 g, 41.35 mmol, 7·20 mL, 3 eq). The mixture was stirred at 25° C. for 12 hr. LC-MS showed desired compound was detected. The reaction mixture was concentrated under reduced pressure to remove solvent. The residue was purified by flash silica gel chromatography (40 g Silica Flash Column, Eluent of 0˜20% Ethyl acetate/Petroleum ether gradient @ 120 mL/min) to give 4-[(2-hydroxy-2-methyl-propyl)amino]-2-methylsulfanyl-pyrimidine-5-carbaldehyde (1.7 g, 6.8 mmol, 49.6% yield) as a light yellow oil.

To a solution of 4-[(2-hydroxy-2-methyl-propyl)amino]-2-methylsulfanyl-pyrimidine-5-carbaldehyde (1.9 g, 7.7 mmol, 1 eq) in DMF (45 mL) was added tert-butyl 4-(2-ethoxy-2-oxo-ethyl)-8-methyl-2,3-dihydroquinoxaline-1-carboxylate (2.6 g, 7.7 mmol, 1 eq) and Cs₂CO₃ (5.0 g, 15.3 mmol, 2 eq). The mixture was stirred at 60° C. for 12 hr. LC-MS showed desired compound was detected. The reaction mixture was concentrated under reduced pressure to remove solvent. The residue was diluted with H₂O (50 mL) and extracted with ethyl acetate (70 mL× 4). The combined organic layers were washed with brine (100 mL× 3), dried over Na₂SO₄, filtered and concentrated under reduced pressure to give a residue. The residue was purified by flash silica gel chromatography (40 g Silica Flash Column, Eluent of 0˜20% Ethyl acetate/Petroleum ether gradient @ 120 mL/min) to give tert-butyl 4-[8-(2-hydroxy-2-methyl-propyl)-2-methylsulfanyl-7-oxo-pyrido[2,3-d]pyrimidin-6-yl]-8-methyl-2,3-dihydroquinoxaline-1-carboxylate (2.5 g) as a yellow solid.

To a solution of tert-butyl 4-[8-(2-hydroxy-2-methyl-propyl)-2-methylsulfanyl-7-oxo-pyrido[2,3-d]pyrimidin-6-yl]-8-methyl-2,3-dihydroquinoxaline-1-carboxylate (1.5 g, 2.9 mmol, 1 eq) in DCM (20 mL) was added m-CPBA (892.8 mg, 4.4 mmol, 85% purity, 1.5 eq) at 0° C. The mixture was stirred at 25° C. for 2 hr. LC-MS showed desired compound was detected. The residue was diluted with Na₂SO₃ (50 mL) and extracted with dichloromethane (50 mL× 4). The combined organic layers were washed with NaHCO₃ (60 mL× 3), dried over Na₂SO₄, filtered and concentrated under reduced pressure to give tert-butyl 4-[8-(2-hydroxy-2-methyl-propyl)-2-methylsulfonyl-7-oxo-pyrido[2,3-d]pyrimidin-6-yl]-8-methyl-2,3-dihydroquinoxaline-1-carboxylate (1.8 g, crude) as a yellow solid.

To a solution of tert-butyl 4-[8-(2-hydroxy-2-methyl-propyl)-2-methylsulfonyl-7-oxo-pyrido[2,3-d]pyrimidin-6-yl]-8-methyl-2,3-dihydroquinoxaline-1-carboxylate (200 mg, 367.9 μmol, 1 eq) in dioxane (2 mL) was added 4-[2-(dimethylamino)ethoxy]aniline (132.6 mg, 735.8 μmol, 2 eq) and TFA (83.9 mg, 735.8 μmol, 54.7 μL, 2 eq). The mixture was stirred at 100° C. for 12 hr (batch× 3). LC-MS showed desired compound was detected. The reaction mixture was concentrated under reduced pressure to remove solvent. The residue was purified by prep-HPLC (TFA condition column: Phenomenex luna C18 100×40 mm×5 μm; mobile phase: [H₂O (0.1% TFA)-ACN]; gradient: 25%-55% B over 8.0 min) to give tert-butyl 4-[2-[4-[2-(dimethylamino)ethoxy]anilino]-8-(2-hydroxy-2-methyl-propyl)-7-oxo-pyrido[2,3-d]pyrimidin-6-yl]-8-methyl-2,3-dihydroquinoxaline-1-carboxylate (462 mg) as a yellow solid.

A mixture of tert-butyl 4-[2-[4-[2-(dimethylamino)ethoxy]anilino]-8-(2-hydroxy-2-methyl-propyl)-7-oxo-pyrido[2,3-d]pyrimidin-6-yl]-8-methyl-2,3-dihydroquinoxaline-1-carboxylate (200 mg, 310.7 μmol, 1 eq) in HCl/EtOAc (2 mL, 4M) was stirred at 25° C. for 1 hr. LC-MS showed desired compound was detected. The reaction mixture was concentrated under reduced pressure to give 2-[4-[2-(dimethylamino)ethoxy]anilino]-8-(2-hydroxy-2-methyl-propyl)-6-(5-methyl-3,4-dihydro-2H-quinoxalin-1-yl)pyrido[2,3-d]pyrimidin-7-one (200 mg, crude) as a yellow solid.

To a solution of 2-[4-[2-(dimethylamino)ethoxy]anilino]-8-(2-hydroxy-2-methyl-propyl)-6-(5-methyl-3,4-dihydro-2H-quinoxalin-1-yl)pyrido[2,3-d]pyrimidin-7-one (120 mg, 206.9 μmol, 1 eq, HCl) in THF (1.5 mL) and H₂O (0.5 mL) was added NaHCO₃ (52.1 mg, 620.6 μmol, 24·2 μL, 3 eq) and prop-2-enoyl chloride (18.7 mg, 206.9 μmol, 16.8 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition column: Phenomenex luna C18 100×40 mm× 5 μm; mobile phase: [H₂O (0.1% TFA)-ACN]; gradient: 15%-45% B over 8.0 min) and (neutral condition column: Waters Xbridge BEH C18 100×30 mm×10 μm; mobile phase: [H₂O (10 mM NH₄HCO₃)-ACN]; gradient: 25%-55% B over 8.0 min) to give 2-[4-[2-(dimethylamino)ethoxy]anilino]-8-(2-hydroxy-2-methyl-propyl)-6-(5-methyl-4-prop-2-enoyl-2,3-dihydroquinoxalin-1-yl)pyrido[2,3-d]pyrimidin-7-one (24.96 mg, 41.76 μmol, 20.19% yield, 100% purity) as a yellow solid.

### Procedure for Preparation of Compound 055

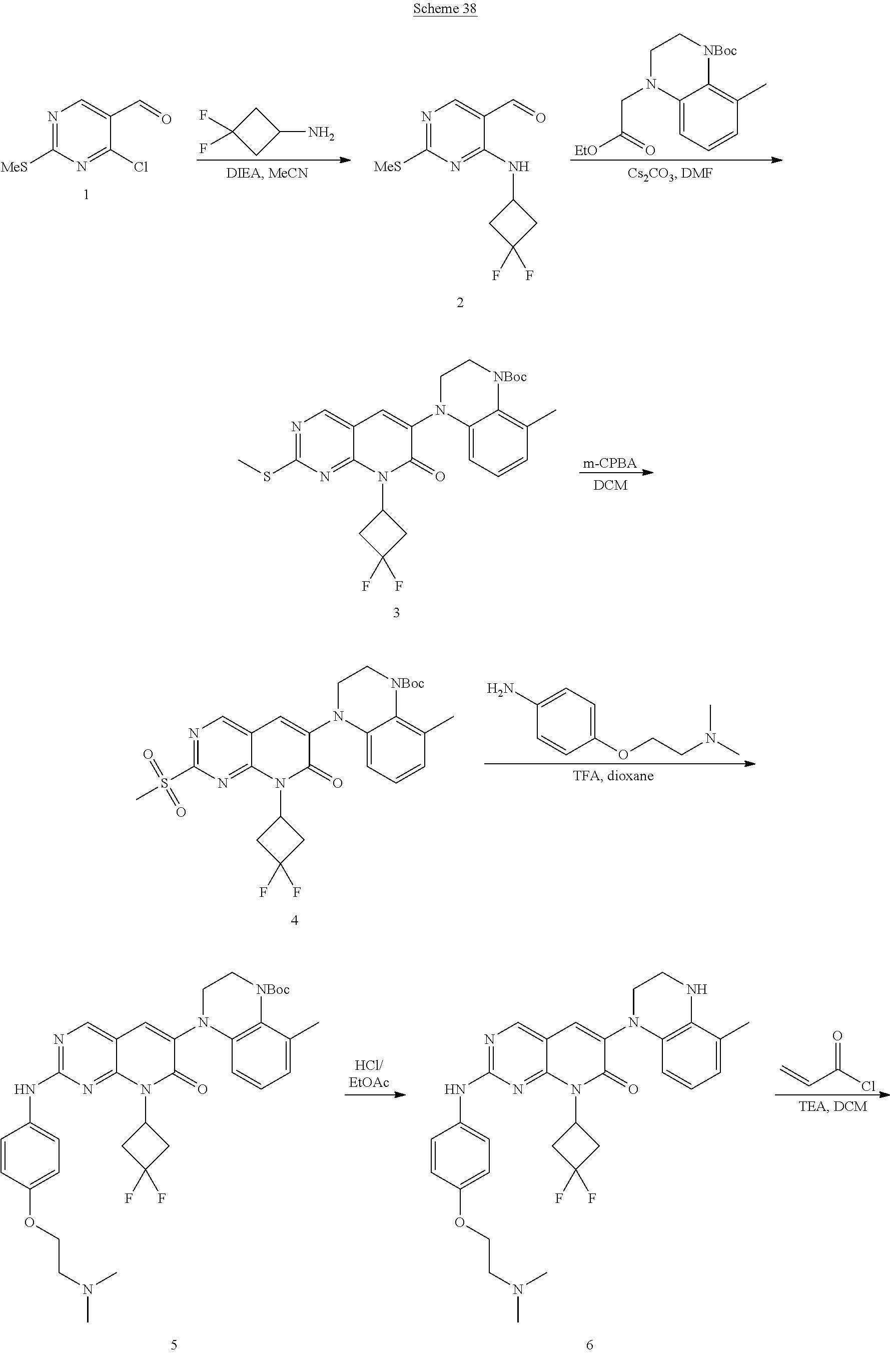

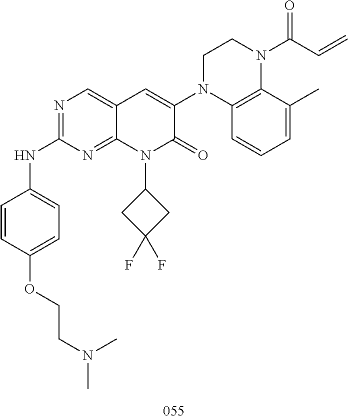 [SMILES: C=CC(=O)N1CCN(c2cc3cnc(Nc4ccc(OCCN(C)C)cc4)nc3n(C3CC(F)(F)C3)c2=O)c2cccc(C)c21]

To a solution of 4-chloro-2-methylsulfanyl-pyrimidine-5-carbaldehyde (1 g, 5.3 mmol, 1 eq) in MeCN (60 mL) was added DIEA (1.3 g, 10.6 mmol, 1.8 mL, 2 eq) to adjust pH=8-9 and 3,3-difluorocyclobutanamine (567.7 mg, 5.3 mmol, 1 eq). The mixture was stirred at 20° C. for 2 hr. LC-MS showed desired compound was detected. The reaction mixture was concentrated under reduced pressure to remove solvent. The residue was diluted with ethyl acetate (30 mL) and extracted with H₂O (20 mL× 3). The combined organic layers were dried over Na₂SO₄, filtered and concentrated under reduced pressure to give a residue. The residue was purified by flash silica gel chromatography (12 g Silica Flash Column, Eluent of 0˜9% Ethyl acetate/Petroleum ether gradient @ 100 mL/min) to give 4-[(3,3-difluorocyclobutyl)amino]-2-methylsulfanyl-pyrimidine-5-carbaldehyde (454 mg, 1.5 mmol, 29.4% yield, 89% purity) as a yellow solid.

To a solution of 4-[(3,3-difluorocyclobutyl)amino]-2-methylsulfanyl-pyrimidine-5-carbaldehyde (414 mg, 1.6 mmol, 1 eq) in DMF (4 mL) was added tert-butyl 4-(2-ethoxy-2-oxo-ethyl)-8-methyl-2,3-dihydroquinoxaline-1-carboxylate (533.9 mg, 1.6 mmol, 1 eq) and Cs₂CO₃ (1.5 g, 4.7 mmol, 3 eq). The mixture was stirred at 40° C. for 12 hr. LC-MS showed desired compound was detected. The reaction mixture was concentrated under reduced pressure to remove solvent. The residue was purified by prep-TLC (SiO₂, Petroleum ether/Ethyl acetate=3/1) to give tert-butyl 4-[8-(3,3-difluorocyclobutyl)-2-methylsulfanyl-7-oxo-pyrido[2,3-d]pyrimidin-6-yl]-8-methyl-2,3-dihydroquinoxaline-1-carboxylate (506 mg, 831·2 μmol, 52% yield, 87% purity) as a yellow solid.

At 0° C., to a solution of tert-butyl 4-[8-(3,3-difluorocyclobutyl)-2-methylsulfanyl-7-oxo-pyrido[2,3-d]pyrimidin-6-yl]-8-methyl-2,3-dihydroquinoxaline-1-carboxylate (506 mg, 955.4 μmol, 1 eq) in DCM (5 mL) was added m-CPBA (290.9 mg, 1.4 mmol, 85% purity, 1.5 eq). The mixture was stirred at 20° C. for 2 hr. LC-MS showed desired compound was detected. The reaction mixture was quenched by addition Na₂SO_{3 30} ml at 0° C., and then extracted with dichloromethane (20 mL×3). The combined organic layers were washed with NaHCO₃ (20 ml× 3), dried over Na₂SO₄, filtered and concentrated under reduced pressure to give tert-butyl 4-[8-(3,3-difluorocyclobutyl)-2-methylsulfonyl-7-oxo-pyrido[2,3-d]pyrimidin-6-yl]-8-methyl-2,3 dihydroquinoxaline-1-carboxylate (609 mg, crude) as an orange solid.

To a solution of tert-butyl 4-[8-(3,3-difluorocyclobutyl)-2-methylsulfonyl-7-oxo-pyrido[2,3-d]pyrimidin-6-yl]-8-methyl-2,3-dihydroquinoxaline-1-carboxylate (260 mg, 462.9 μmol, 1 eq) in dioxane (2.6 mL) was added TFA (105.5 mg, 925.9 μmol, 68.7 μL, 2 eq) and 4-[2-(dimethylamino)ethoxy]aniline (166.8 mg, 925.9 μmol, 2 eq). The mixture was stirred at 120° C. for 12 hr. LC-MS showed desired compound was detected. The reaction mixture was concentrated under reduced pressure to remove solvent. The residue was purified by prep-HPLC (TFA condition: column: Phenomenex luna C18 100×40 mm× 5 μm; mobile phase: [H₂O (0.1% TFA)-ACN]; gradient: 40%-70% B over 8.0 min) to give tert-butyl 4-[8-(3,3-difluorocyclobutyl)-2-[4-[2-(dimethylamino)ethoxy]anilino]-7-oxo-pyrido[2,3-d]pyrimidin-6-yl]-8-methyl-2,3-dihydroquinoxaline-1-carboxylate (129 mg, 95% purity) as black oil.

A solution of tert-butyl 4-[8-(3,3-difluorocyclobutyl)-2-[4-[2-(dimethylamino)ethoxy]anilino]-7-oxo-pyrido[2,3-d]pyrimidin-6-yl]-8-methyl-2,3-dihydroquinoxaline-1-carboxylate (129 mg, 194.9 μmol, 1 eq) in EtOAc (1 mL) and HCl/EtOAc (1 mL, 4 M). The mixture was stirred at 20° C. for 1 hr. LC-MS showed desired compound was detected. The reaction mixture was concentrated under reduced pressure to give 8-(3,3-difluorocyclobutyl)-2-[4-[2-(dimethylamino)ethoxy]anilino]-6-(5-methyl-3,4-dihydro-2H-quinoxalin-1-yl)pyrido[2,3-d]pyrimidin-7-one (120 mg, crude) as a yellow solid.

To a solution of 8-(3,3-difluorocyclobutyl)-2-[4-[2-(dimethylamino)ethoxy]anilino]-6-(5-methyl-3,4-dihydro-2H-quinoxalin-1-yl)pyrido[2,3-d]pyrimidin-7-one (80 mg, 142.4 μmol, 1 eq) in DCM (2 mL) was added dropwise TEA (43·2 mg, 427.3 μmol, 59.4 μL, 3 eq) and prop-2-enoyl chloride (12.8 mg, 142.4 μmol, 11.5 μL, 1 eq) in DCM (0.1 mL) at 0° C. The mixture was stirred at 0° C. for 30 min. LC-MS showed desired compound was detected. The reaction mixture was concentrated under N₂ to remove solvent. The residue was purified by prep-HPLC (neutral condition column: Waters Xbridge BEH C18 100×30 mm×10 μm; mobile phase: [H₂O (10 mM NH₄HCO₃)-ACN]; gradient: 40%-70% B over 8.0 min) to give 8-(3,3-difluorocyclobutyl)-2-[4-[2-(dimethylamino)ethoxy]anilino]-6-(5-methyl-4-prop-2-enoyl-2,3-dihydroquinoxalin-1-yl)pyrido[2,3-d]pyrimidin-7-one (4.8 mg, 7.6 μmol, 5.3% yield, 97% purity) as a yellow solid.

### Procedure for Preparation of Compound 071

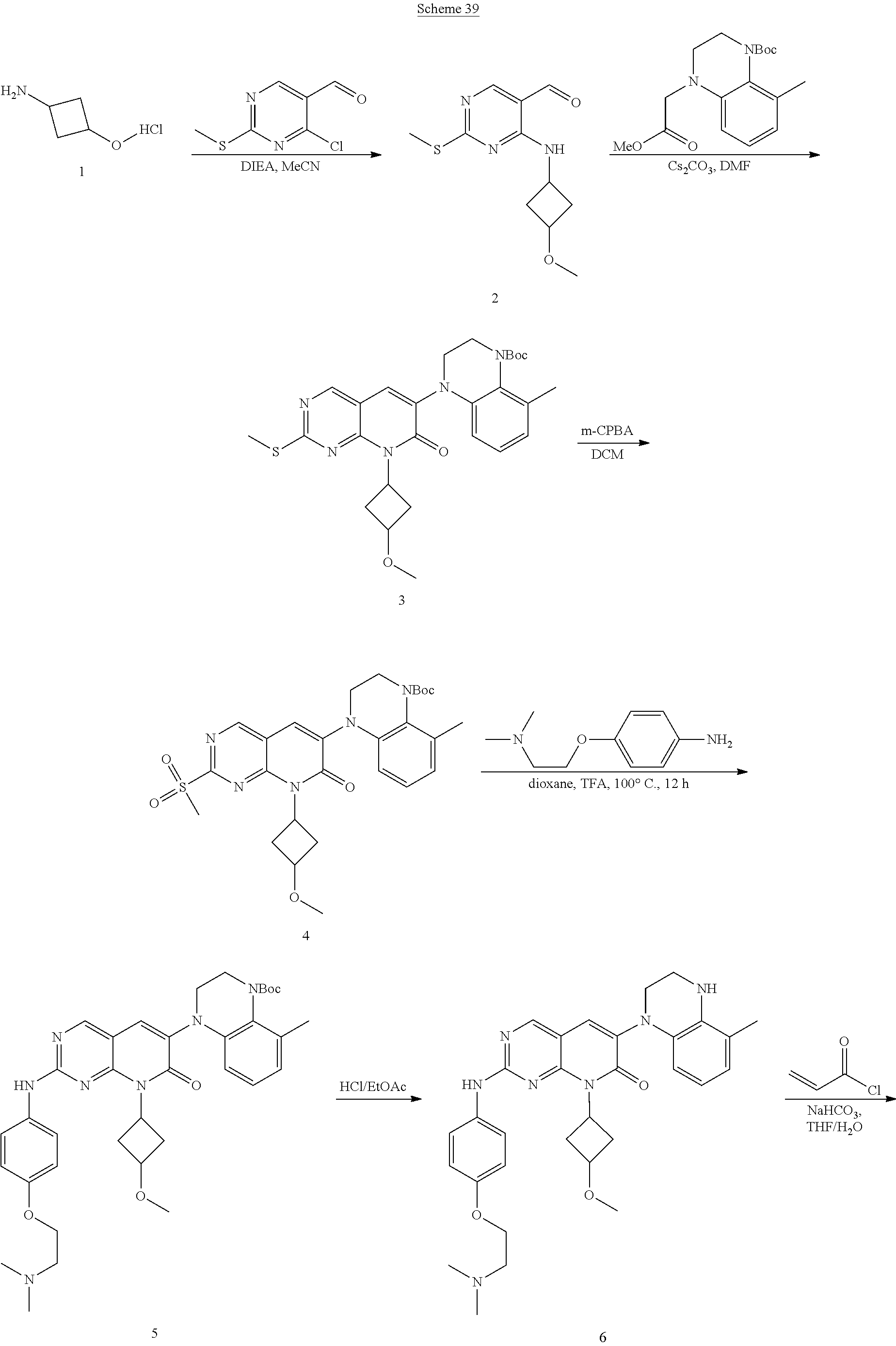

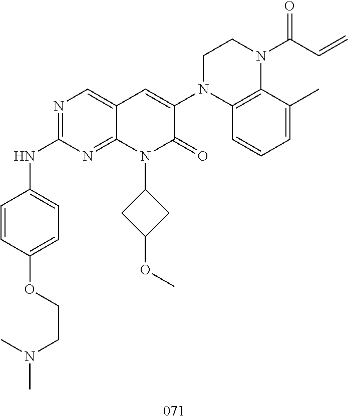 [SMILES: C=CC(=O)N1CCN(c2cc3cnc(Nc4ccc(OCCN(C)C)cc4)nc3n(C3CC(OC)C3)c2=O)c2cccc(C)c21]

To a solution of 4-chloro-2-methylsulfanyl-pyrimidine-5-carbaldehyde (1.5 g, 7.8 mmol, 1.1 eq) in MeCN (20 mL) was added DIEA (4.6 g, 35.3 mmol, 6.1 mL, 5 eq) and 3-methoxycyclobutanamine (1.0 g, 7.0 mmol, 1 eq, HCl). The mixture was stirred at 20° C. for 12 hr. LC-MS showed desired compound was detected. The reaction mixture was concentrated under reduced pressure to remove solvent. The residue was purified by flash silica gel chromatography (20 g Silica Flash Column, Eluent of 0˜3% Ethyl acetate/Petroleum ether gradient @ 120 mL/min) to give 4-[(3-methoxycyclobutyl)amino]-2-methylsulfanyl-pyrimidine-5-carbaldehyde (1.3 g, 5.13 mmol, 72.80% yield) as a light yellow solid.

To a solution of tert-butyl 4-(2-ethoxy-2-oxo-ethyl)-8-methyl-2,3-dihydroquinoxaline-1-carboxylate (1.4 g, 4.3 mmol, 1 eq) in DMF (25 mL) was added Cs₂CO₃ (4·2 g, 13.0 mmol, 3 eq) and 4-[(3-methoxycyclobutyl)amino]-2-methylsulfanyl-pyrimidine-5-carbaldehyde (1.1 g, 4.3 mmol, 1 eq). The mixture was stirred at 60° C. for 12 hr. LC-MS showed desired compound was detected. The reaction mixture was concentrated under reduced pressure to remove solvent. The residue was diluted with H₂O 20 mL and extracted with ethyl acetate (20 mL×3). The combined organic layers were washed with brine (15 mL×1), dried over Na₂SO₄, filtered and concentrated under reduced pressure. The residue was purified by flash silica gel chromatography (20 g Silica Flash Column, Eluent of 0˜10% Ethyl acetate/Petroleum ether gradient @ 120 mL/min) to give tert-butyl 4-[8-(3-methoxycyclobutyl)-2-methylsulfanyl-7-oxo-pyrido[2,3-d]pyrimidin-6-yl]-8-methyl-2,3-dihydroquinoxaline-1-carboxylate (824 mg, 1.6 mmol, 36·2% yield) as a yellow solid.

To a solution of tert-butyl 4-[8-(3-methoxycyclobutyl)-2-methylsulfanyl-7-oxo-pyrido[2,3-d]pyrimidin-6-yl]-8-methyl-2,3-dihydroquinoxaline-1-carboxylate (424 mg, 809.7 μmol, 1 eq) in DCM (10 mL) was added m-CPBA (295.9 mg, 1.5 mmol, 85% purity, 1.8 eq) at 0° C. The mixture was stirred at 20° C. for 1 hr. LC-MS showed desired compound was detected. The reaction mixture was partitioned between Na₂SO₃ (100 mL) and dichloromethane (100×3 mL). The organic phase was separated, washed with NaHCO₃ (80 mL× 3), dried over Na₂SO₄, filtered and concentrated under reduced pressure to give tert-butyl 4-[8-(3-methoxycyclobutyl)-2-methylsulfonyl-7-oxo-pyrido[2,3-d]pyrimidin-6-yl]-8-methyl-2,3-dihydroquinoxaline-1-carboxylate (400 mg, crude) as a yellow solid.

To a solution of tert-butyl 4-[8-(3-methoxycyclobutyl)-2-methylsulfonyl-7-oxo-pyrido[2,3-d]pyrimidin-6-yl]-8-methyl-2,3-dihydroquinoxaline-1-carboxylate (200 mg, 359.9 μmol, 1 eq) in dioxane (2 mL) was added TFA (61.6 mg, 539.9 μmol, 40.1 μL, 1.5 eq) and 4-[2-(dimethylamino)ethoxy]aniline (97.3 mg, 539.9 μmol, 1.5 eq). The mixture was stirred at 100° C. for 12 hr. LC-MS showed desired compound was detected. The reaction mixture was concentrated under reduced pressure to remove solvent. The residue was purified by prep-HPLC (TFA condition; column: Phenomenex luna C18 80×30 mm×3 μm; mobile phase: [H₂O (0.1% TFA)-ACN]; gradient: 30%-60% B over 8.0 min) to give tert-butyl 4-[2-[4-[2-(dimethylamino)ethoxy]anilino]-8-(3-methoxycyclobutyl)-7-oxo-pyrido[2,3-d]pyrimidin-6-yl]-8-methyl-2,3-dihydroquinoxaline-1-carboxylate (200 mg, 305.0 μmol, 42.4% yield) as an orange solid.

To a solution of tert-butyl 4-[2-[4-[2-(dimethylamino)ethoxy]anilino]-8-(3-methoxycyclobutyl)-7-oxo-pyrido[2,3-d]pyrimidin-6-yl]-8-methyl-2,3-dihydroquinoxaline-1-carboxylate (100 mg, 152.5 μmol, 1 eq) in EtOAc (1 mL) was added HCl/EtOAc (1 mL, 4 M).

The mixture was stirred at 20° C. for 1 hr. LC-MS showed desired compound was detected. The reaction mixture was filtered and concentrated under reduced pressure to give 2-[4-[2-(dimethylamino)ethoxy]anilino]-8-(3-methoxycyclobutyl)-6-(5-methyl-3,4-dihydro-2H-quinoxalin-1-yl)pyrido[2,3-d]pyrimidin-7-one (87 mg, crude, HCl) as a yellow solid.

To a solution of 2-[4-[2-(dimethylamino)ethoxy]anilino]-8-(3-methoxycyclobutyl)-6-(5-methyl-3,4-dihydro-2H-quinoxalin-1-yl)pyrido[2,3-d]pyrimidin-7-one (60 mg, 108.0 μmol, 1 eq) in THF (1.5 mL) and H₂O (0.5 mL) was added NaHCO₃ (36.3 mg, 431.9 μmol, 16.8 μL, 4 eq) and prop-2-enoyl chloride (9.8 mg, 108.0 μmol, 8.8 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition; column: Phenomenex luna C18 80×30 mm×3 μm; mobile phase: [H₂O (0.1% TFA)-ACN]; gradient: 20%-50% B over 8.0 min) to give 2-[4-[2-(dimethylamino)ethoxy]anilino]-8-(3-methoxycyclobutyl)-6-(5-methyl-4-prop-2-enoyl-2,3-dihydroquinoxalin-1-yl)pyrido[2,3-d]pyrimidin-7-one (23.3 mg, 31.9 μmol, 29.5% yield, 99.0% purity, TFA) as a yellow solid

### Procedure for Preparation of Compound 066

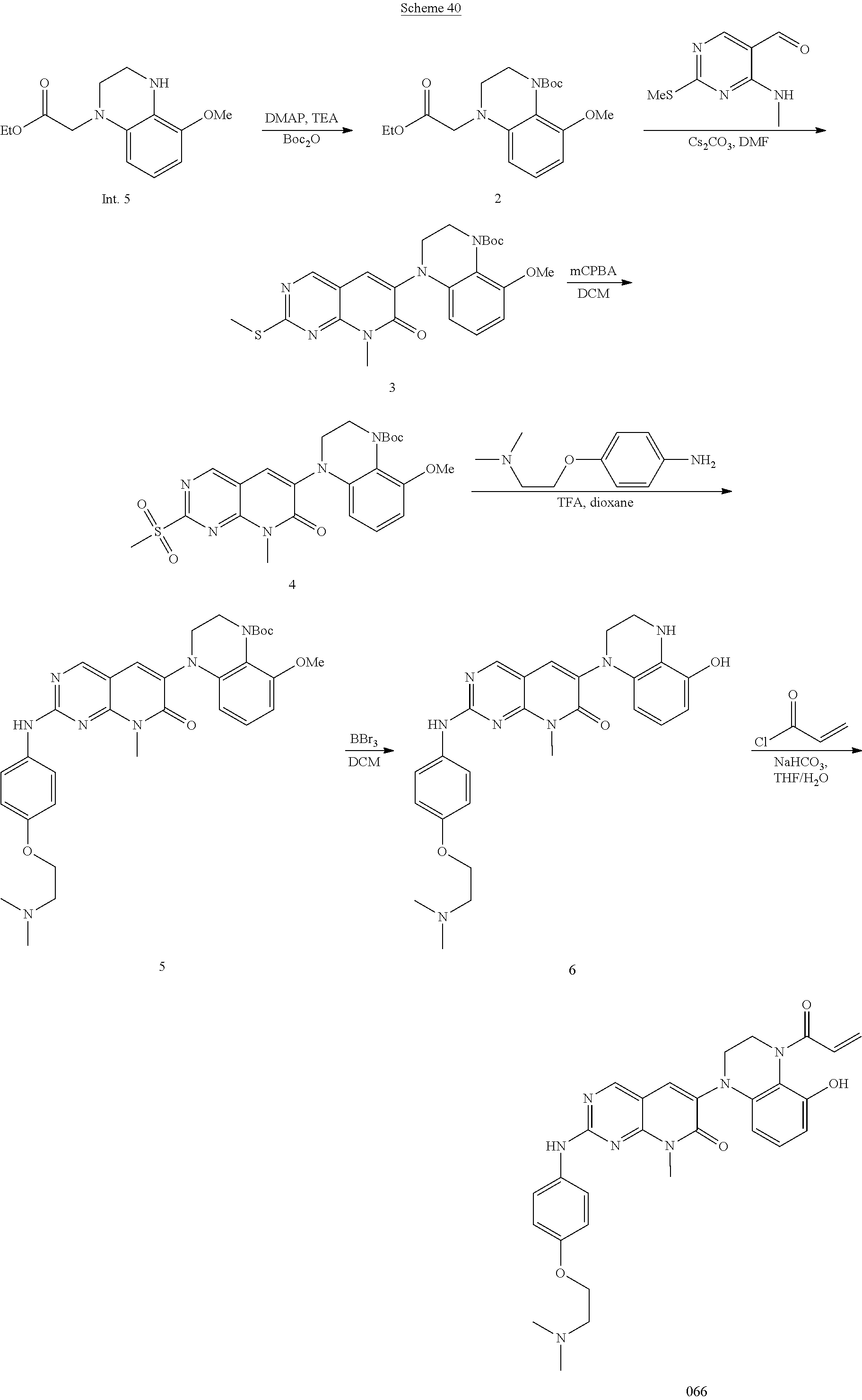

To a solution of ethyl 2-(5-methoxy-3,4-dihydro-2H-quinoxalin-1-yl)acetate (1·2 g, 4.7 mmol, 1 eq) in Boc₂O (12 mL) was added DMAP (58.5 mg, 479.4 μmol, 0.1 eq) and TEA (2.4 g, 23.9 mmol, 3.3 mL, 5 eq). The mixture was stirred at 80° C. for 12 hr. LC-MS showed desired compound was detected. The reaction mixture was concentrated under reduced pressure to remove solvent. The residue was purified by flash silica gel chromatography (20 g Silica Flash Column, Eluent of 0˜20% Ethyl acetate/Petroleum ether gradient @ 100 mL/min) to give tert-butyl 4-(2-ethoxy-2-oxo-ethyl)-8-methoxy-2,3-dihydroquinoxaline-1-carboxylate (1.1 g, 2.5 mmol, 53.8% yield, 78% purity) as yellow oil.

To a solution of tert-butyl 4-(2-ethoxy-2-oxo-ethyl)-8-methoxy-2,3-dihydroquinoxaline-1-carboxylate (183.3 mg, 523.1 μmol, 1 eq) in DMF (7 mL) was added 4-(methylamino)-2-methylsulfanyl-pyrimidine-5-carbaldehyde (95.8 mg, 523.1 μmol, 1 eq) and Cs₂CO₃ (340.8 mg, 1.0 mmol, 2 eq). The mixture was stirred at 60° C. for 2 hr (batch×2). LC-MS showed desired compound was detected. The residue was diluted with H₂O (50 mL) and extracted with ethyl acetate (30 mL×3). The combined organic layers were washed with brine (20 mL× 3), dried over Na₂SO₄, filtered and concentrated under reduced pressure to give a residue. The residue was purified by prep-TLC (SiO₂, Petroleum ether: Ethyl acetate=1:1) to give tert-butyl 8-methoxy-4-(8-methyl-2-methylsulfanyl-7-oxo-pyrido[2,3-d]pyrimidin-6-yl)-2,3-dihydroquinoxaline-1-carboxylate (435 mg, 474.8 μmol, 90.7% yield, 91% purity) as a yellow solid.

To a solution of tert-butyl 8-methoxy-4-(8-methyl-2-methylsulfanyl-7-oxo-pyrido[2,3-d]pyrimidin-6-yl)-2,3-dihydroquinoxaline-1-carboxylate (435 mg, 926.4 μmol, 1 eq) in DCM (5 mL) was added m-CPBA (282.1 mg, 1.3 mmol, 85% purity, 1.5 eq) at 0° C. The resulting mixture was stirred at 25° C. for 2 hr. LC-MS showed desired compound was detected. The reaction mixture was quenched by addition Na₂SO₃ (15 mL), extracted with Dichloromethane (10 mL× 3). The combined organic layers were washed with NaHCO₃ (10 mL× 2), and dried over Na₂SO₄, filtered and concentrated under reduced pressure to give tert-butyl 8-methoxy-4-(8-methyl-2-methylsulfinyl-7-oxo-pyrido[2,3-d]pyrimidin-6-yl)-2,3-dihydroquinoxaline-1-carboxylate (493.4 mg, crude) as a yellow solid.

To a solution of tert-butyl 8-methoxy-4-(8-methyl-2-methylsulfinyl-7-oxo-pyrido[2,3-d]pyrimidin-6-yl)-2,3-dihydroquinoxaline-1-carboxylate (246.7 mg, 508.0 μmol, 1 eq) in dioxane (5 mL) was added TFA (86.9 mg, 762.1 μmol, 56.6 μL, 1.5 eq) and 4-[2-(dimethylamino)ethoxy]aniline (109.9 mg, 609.6 μmol, 1·2 eq). The mixture was stirred at 120° C. for 12 hr. LC-MS showed desired compound was detected. The reaction mixture was concentrated under reduced pressure to remove solvent. The residue was purified by prep-HPLC (neutral condition; column: Waters Xbridge BEH C18 100×30 mm×10 μm; mobile phase: [H₂O (10 mM NH₄HCO₃)-ACN]; gradient: 35%-65% B over 8.0 min) to give tert-butyl 4-[2-[4-[2-(dimethylamino)ethoxy]anilino]-8-methyl-7-oxo-pyrido[2,3-d]pyrimidin-6-yl]-8-methoxy-2,3-dihydroquinoxaline-1-carboxylate (330 mg, 504.5 μmol, 49.6% yield, 92% purity) as a yellow solid.

To a solution of tert-butyl 4-[2-[4-[2-(dimethylamino)ethoxy]anilino]-8-methyl-7-oxo-pyrido[2,3-d]pyrimidin-6-yl]-8-methoxy-2,3-dihydroquinoxaline-1-carboxylate (30 mg, 49.8 μmol, 1 eq) in DCM (3 mL) was added BBr₃ (1.3 g, 5.1 mmol, 0.5 mL, 104.0 eq) at 0° C. The mixture was stirred at 0° C. for 1 hr. LC-MS showed desired compound was detected. The reaction mixture was quenched by addition methanol (15 mL) at 0° C., and then was concentrated under reduced pressure to remove solvent. The residue was purified by prep-HPLC (TFA condition; column: Phenomenex luna C18 100×40 mm×5 μm; mobile phase: [H₂O (0.1% TFA)-ACN]; gradient: 1%-20% B over 8.0 min) to give 2-[4-[2-(dimethylamino)ethoxy]anilino]-6-(5-hydroxy-3,4-dihydro-2H-quinoxalin-1-yl)-8-methyl-pyrido[2,3-d]pyrimidin-7-one (30 mg, 61.5 μmol, 24.6% yield) as a yellow solid.

To a solution of 2-[4-[2-(dimethylamino)ethoxy]anilino]-6-(5-hydroxy-3,4-dihydro-2H-quinoxalin-1-yl)-8-methyl-pyrido[2,3-d]pyrimidin-7-one (10 mg, 20.5 μmol, 1 eq) in H₂O (0·2 mL) and THF (0.8 mL) was added NaHCO₃ (5.1 mg, 61.5 μmol, 2.3 μL, 3 eq). The mixture was added dropwise prop-2-enoyl chloride (1.8 mg, 20.5 μmol, 1.6 μL, 1 eq) in THF (0.1 mL) at 0° C. and stirred at 0° C. for 0.5 hr. LC-MS showed desired compound was detected. The reaction mixture was concentrated under reduced pressure to remove solvent. The residue was purified by prep-HPLC (TFA condition; column: Phenomenex luna C18 100×40 mm×5 μm; mobile phase: [H₂O (0.1% TFA)-ACN];gradient: 1%-35% B over 8.0 min) to give 2-[4-[2-(dimethylamino)ethoxy]anilino]-6-(5-hydroxy-4-prop-2-enoyl-2,3-dihydroquinoxalin-1-yl)-8-methyl-pyrido[2,3-d]pyrimidin-7-one (9.5 mg, 17.6 μmol, 28.6% yield) as a yellow solid.

### Procedure for Preparation of Compound 072

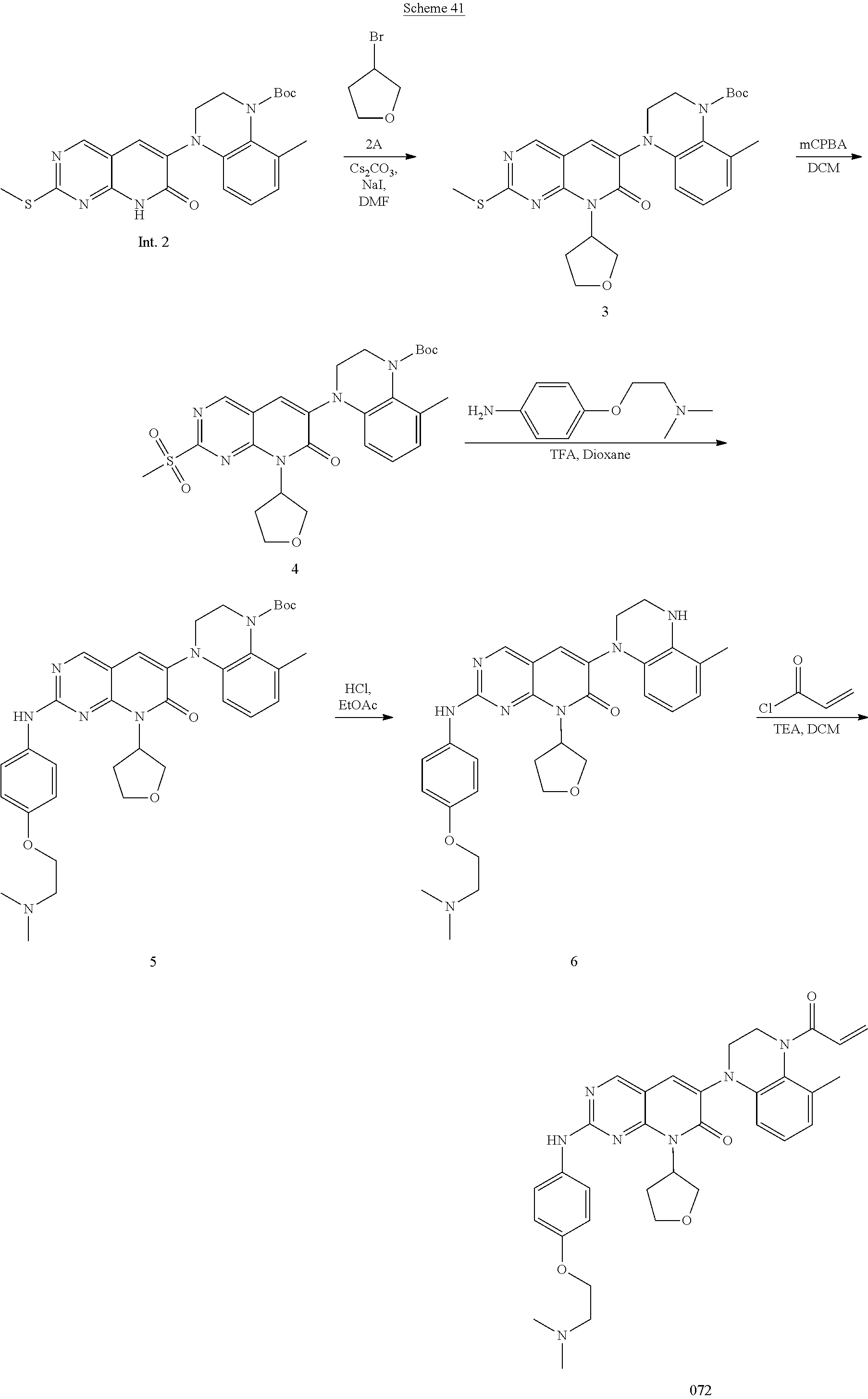

To a solution of tert-butyl 8-methyl-4-(2-methylsulfanyl-7-oxo-8H-pyrido[2,3-d]pyrimidin-6-yl)-2,3-dihydroquinoxaline-1-carboxylate (1 g, 2.3 mmol, 1 eq) and 3-bromotetrahydrofuran (515.3 mg, 3.4 mmol, 1.5 eq) in DMF (20 mL) was added Cs₂CO₃ (2·2 g, 6.8 mmol, 3 eq) and NaI (68·2 mg, 455.0 μmol, 0·2 eq). The mixture was stirred at 90° C. for 12 hr. LC-MS showed desired compound was detected. The reaction mixture was concentrated under reduced pressure to remove solvent. The residue was diluted with H₂O (100 mL) and extracted with ethyl acetate (100 mL× 3). The combined organic layers were washed with brine (80 mL× 1), dried over Na₂SO₄, filtered and concentrated under reduced pressure. The residue was purified by flash silica gel chromatography (20 g Silica Flash Column, Eluent of 0˜18% Ethyl acetate/Petroleum ether gradient @ 100 mL/min) to give tert-butyl 8-methyl-4-(2-methylsulfanyl-7-oxo-8-tetrahydrofuran-3-yl-pyrido[2,3-d]pyrimidin-6-yl)-2,3-dihydroquinoxaline-1-carboxylate (560 mg, 1.1 mmol, 48.3% yield) as a yellow solid.

To a solution of tert-butyl 8-methyl-4-(2-methylsulfanyl-7-oxo-8-tetrahydrofuran-3-yl-pyrido[2,3-d]pyrimidin-6-yl)-2,3-dihydroquinoxaline-1-carboxylate (300 mg, 588.7 μmol, 1 eq) in DCM (5 mL) was added m-CPBA (215.1 mg, 1.1 mmol, 85% purity, 1.8 eq) at 0° C. The mixture was stirred at 20° C. for 12 hr. LC-MS showed desired compound was detected. The reaction mixture was partitioned between Na₂SO₃ (60 mL) and dichloromethane (60×3 mL). The organic phase was separated, washed with NaHCO₃ (50 mL× 3), dried over Na₂SO₄, filtered and concentrated under reduced pressure to give tert-butyl 8-methyl-4-(2-methylsulfonyl-7-oxo-8-tetrahydrofuran-3-yl-pyrido[2,3-d]pyrimidin-6-yl)-2,3-dihydroquinoxaline-1-carboxylate (350 mg, crude) as a yellow solid.

To a solution of tert-butyl 8-methyl-4-(2-methylsulfonyl-7-oxo-8-tetrahydrofuran-3-yl-pyrido[2,3-d]pyrimidin-6-yl)-2,3-dihydroquinoxaline-1-carboxylate (300 mg, 277.0 μmol, 1 eq) in dioxane (3 mL) was added TFA (47.4 mg, 415.4 μmol, 30.9 μL, 1.5 eq) and 4-[2-(dimethylamino)ethoxy]aniline (74.9 mg, 415.4 μmol, 1.5 eq). The mixture was stirred at 100° C. for 12 hr. LC-MS showed desired compound was detected. The reaction mixture was concentrated under reduced pressure to remove solvent. The residue was purified by prep-HPLC (TFA condition; column: Phenomenex luna C18 100×40 mm× 5 μm; mobile phase: [H₂O (0.1% TFA)-ACN]; gradient: 25%-55% B over 8.0 min) to give tert-butyl 4-[2-[4-[2-(dimethylamino)ethoxy]anilino]-7-oxo-8-tetrahydrofuran-3-yl-pyrido[2,3-d]pyrimidin-6-yl]-8-methyl-2,3-dihydroquinoxaline-1-carboxylate (156 mg, 243.1 μmol, 43.9% yield) as a yellow solid.

To a solution of tert-butyl 4-[2-[4-[2-(dimethylamino)ethoxy]anilino]-7-oxo-8-tetrahydrofuran-3-yl-pyrido[2,3-d]pyrimidin-6-yl]-8-methyl-2,3-dihydroquinoxaline-1-carboxylate (156 mg, 243.08 μmol, 1 eq) in EtOAc (1 mL) was added HCl/EtOAc (1 mL, 4 M). The mixture was stirred at 20° C. for 1 hr. LC-MS showed desired compound was detected. The reaction mixture was filtered and concentrated under reduced pressure to give 2-[4-[2-(dimethylamino)ethoxy]anilino]-6-(5-methyl-3,4-dihydro-2H-quinoxalin-1-yl)-8-tetrahydrofuran-3-yl-pyrido[2,3-d]pyrimidin-7-one (130 mg, crude, HCl) as a yellow solid.

To a solution of 2-[4-[2-(dimethylamino)ethoxy]anilino]-6-(5-methyl-3,4-dihydro-2H-quinoxalin-1-yl)-8-tetrahydrofuran-3-yl-pyrido[2,3-d]pyrimidin-7-one (100 mg, 184.6 μmol, 1 eq) in DCM (2 mL) was added TEA (56.0 mg, 553.9 μmol, 77.1 L, 3 eq) and prop-2-enoyl chloride (16.7 mg, 184.6 μmol, 15.0 μL, 1 eq) at 0° C. The mixture was stirred at 0° C. for 50 min. LC-MS showed desired compound was detected. The reaction mixture was concentrated under column: Phenomenex luna C18 100×40 mm×5 μm; mobile phase: [H₂O (0.1% TFA)-ACN]; gradient: 10%-45% B over 8.0 min) to give 2-[4-[2-(dimethylamino)ethoxy]anilino]-6-(5-methyl-4-prop-2-enoyl-2,3-dihydroquinoxalin-1-yl)-8-tetrahydrofuran-3-yl-pyrido[2,3-d]pyrimidin-7-one (64.4 mg, 90.7 μmol, 49·2% yield, 100% purity, TFA) was obtained as a yellow solid.

### Procedure for Preparation of Compound 078

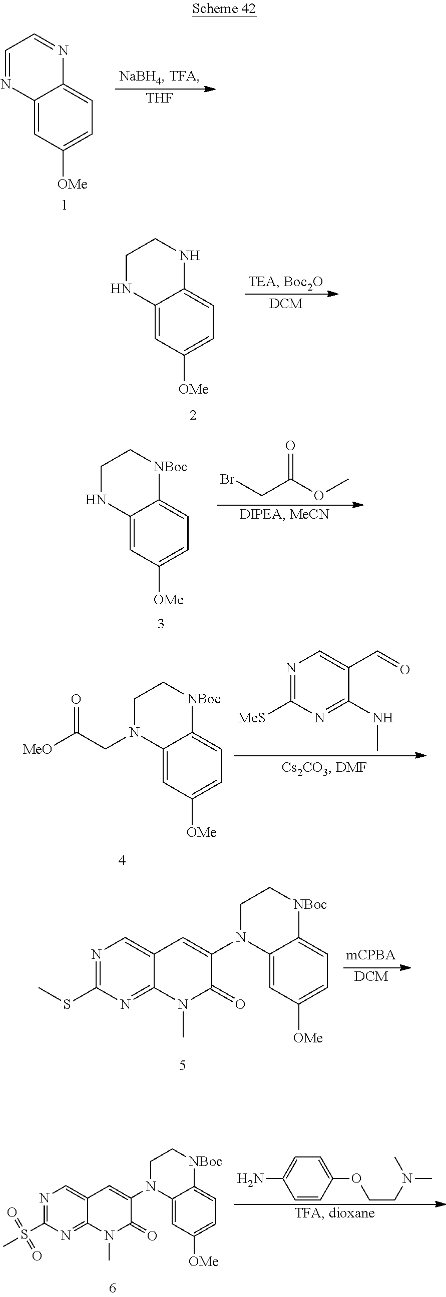

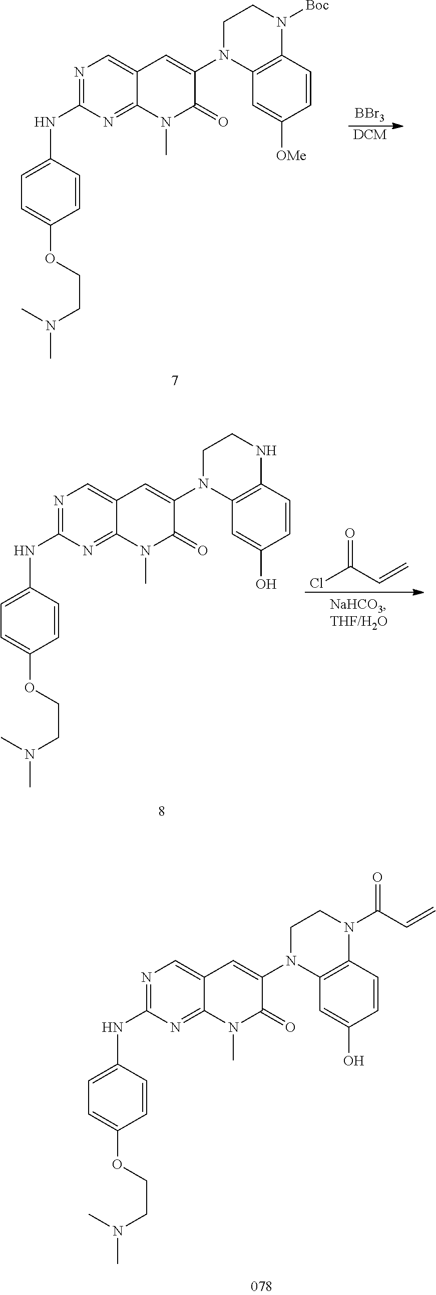

To a solution of 6-methoxyquinoxaline (2 g, 12.49 mmol, 1 eq) in THF (30 mL) was added NaBH₄ (1.7 g, 43.7 mmol, 3.5 eq) at 0° C. The mixture was stirred at 0° C. for 30 min. Then TFA (2.6 g, 22.5 mmol, 1.7 mL, 1.8 eq) was added to the mixture. The mixture was stirred at 0° C. for 30 min. LC-MS showed desired compound was detected. The reaction mixture was quenched by addition NH₄CI (200 mL) at 0° C., and then extracted with ethyl acetate (200 ml×3). The combined organic layers were washed with brine (100 mL× 3), dried over Na₂SO₄, filtered and concentrated under reduced pressure to give a residue. The residue was purified by flash silica gel chromatography (20 g Silica Flash Column, Eluent of 0˜36% Ethyl acetate/Petroleum ether gradient @ 120 mL/min) to give 6-methoxy-1,2,3,4-tetrahydroquinoxaline (2 g, 12·2 mmol, 97.5% yield) as a brown solid.

To a solution of 6-methoxy-1,2,3,4-tetrahydroquinoxaline (300 mg, 1.8 mmol, 1 eq) in DCM (10 mL) was added Boc₂O (478.5 mg, 2·2 mmol, 503.7 μL, 1·2 eq) and TEA (554.6 mg, 5.5 mmol, 762.9 μL, 3 eq). The mixture was stirred at 35° C. for 1 hr. LC-MS showed desired compound was detected. The reaction mixture was concentrated under reduced pressure to remove solvent. The residue was purified by flash silica gel chromatography (4 g Silica Flash Column, Eluent of 0˜7% Ethyl acetate/Petroleum ether gradient @ 80 mL/min) to give tert-butyl 6-methoxy-3,4-dihydro-2H-quinoxaline-1-carboxylate (400 mg, 1.5 mmol, 82.8% yield) as a yellow solid.

To a solution of tert-butyl 6-methoxy-3,4-dihydro-2H-quinoxaline-1-carboxylate (2·2 g, 8.5 mmol, 1 eq) in MeCN (20 mL) was added DIPEA (2·2 g, 17.0 mmol, 3.0 mL, 2 eq) and methyl 2-bromoacetate (1.6 g, 10·2 mmol, 962.7 μL, 1·2 eq). The mixture was stirred at 80° C. for 16 hr. LC-MS showed desired compound was detected. The reaction mixture was concentrated under reduced pressure to remove solvent. The residue was purified by flash silica gel chromatography (20 g Silica Flash Column, Eluent of 0˜6% Ethyl acetate/Petroleum ether gradient @ 120 mL/min) to give tert-butyl 6-methoxy-4-(2-methoxy-2-oxo-ethyl)-2,3-dihydroquinoxaline-1-carboxylate (2.5 g, 7.3 mmol, 86.6% yield) as a white solid.

To a solution of 4-(methylamino)-2-methylsulfanyl-pyrimidine-5-carbaldehyde (544.7 mg, 3.0 mmol, 1 eq) in DMF (10 mL) was added Cs₂CO₃ (2.9 g, 8.9 mmol, 3 eq) and tert-butyl 6-methoxy-4-(2-methoxy-2-oxo-ethyl)-2,3-dihydroquinoxaline-1-carboxylate (1 g, 3.0 mmol, 1 eq). The mixture was stirred at 40° C. for 12 hr. LC-MS showed desired compound was detected. The reaction mixture was concentrated under reduced pressure to remove solvent. The residue was diluted with H₂O (30 mL) and extracted with ethyl acetate (30 mL×3). The combined organic layers were washed with brine (20 mL× 2), dried over Na₂SO₄, filtered and concentrated under reduced pressure to give a residue. The residue was purified by flash silica gel chromatography (12 g Silica Flash Column, Eluent of 0˜40% Ethyl acetate/Petroleum ether gradient @ 100 mL/min) to give tert-butyl 6-methoxy-4-(8-methyl-2-methylsulfanyl-7-oxo-pyrido[2,3-d]pyrimidin-6-yl)-2,3-dihydroquinoxaline-1-carboxylate (760 mg, 1.6 mmol, 54.4% yield) as a yellow solid.

To a solution of tert-butyl 6-methoxy-4-(8-methyl-2-methylsulfanyl-7-oxo-pyrido[2,3-d]pyrimidin-6-yl)-2,3-dihydroquinoxaline-1-carboxylate (760 mg, 1.6 mmol, 1 eq) in DCM (15 mL) was added m-CPBA (492.9 mg, 2.4 mmol, 85% purity, 1.5 eq) at 0° C. The mixture was stirred at 20° C. for 2 hr. LC-MS showed desired compound was detected. The reaction mixture was partitioned between Na₂SO₃ (30 mL) and dichloromethane (30×3 mL). The organic phase was separated, washed with NaHCO₃ (20 mL×3), dried over Na₂SO₄, filtered and concentrated under reduced pressure to give tert-butyl 6-methoxy-4-(8-methyl-2-methylsulfonyl-7-oxo-pyrido[2,3-d]pyrimidin-6-yl)-2,3-dihydroquinoxaline-1-carboxylate (880 mg, crude) as a yellow solid.

To a solution of tert-butyl 6-methoxy-4-(8-methyl-2-methylsulfonyl-7-oxo-pyrido[2,3-d]pyrimidin-6-yl)-2,3-dihydroquinoxaline-1-carboxylate (855 mg, 568·2 μmol, 1 eq) in dioxane (3 mL) was added TFA (291.6 mg, 852.4 μmol, 63.3 μL, 1.5 eq) and 4-[2-(dimethylamino)ethoxy]aniline (445·2 mg, 1.0 mmol, 1.8 eq). The mixture was stirred at 110° C. for 12 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min) to give tert-butyl 4-[2-[4-[2-(dimethylamino)ethoxy]anilino]-8-methyl-7-oxo-pyrido[2,3-d]pyrimidin-6-yl]-6-methoxy-2,3-dihydroquinoxaline-1-carboxylate (520 mg, 864·2 μmol, 50.7% yield) as a yellow solid.

To a solution of tert-butyl 4-[2-[4-[2-(dimethylamino)ethoxy]anilino]-8-methyl-7-oxo-pyrido[2,3-d]pyrimidin-6-yl]-6-methoxy-2,3-dihydroquinoxaline-1-carboxylate (200 mg, 332.4 μmol, 1 eq) in DCM (20 mL) was added BBr₃ (5·2 g, 20.8 mmol, 2.0 mL, 62.5 eq) at 0° C. The mixture was stirred at 0° C. for 1 hr. LC-MS showed desired compound was detected. The reaction mixture was quenched by addition methanol (2 mL) at 0° C., and then was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 1%-25% B over 8.0 min) to give 2-[4-[2-(dimethylamino)ethoxy]anilino]-6-(7-hydroxy-3,4-dihydro-2H-quinoxalin-1-yl)-8-methyl-pyrido[2,3-d]pyrimidin-7-one (140 mg, 287·2 μmol, 86.4% yield) as a yellow solid.

To a solution of 2-[4-[2-(dimethylamino)ethoxy]anilino]-6-(7-hydroxy-3,4-dihydro-2H-quinoxalin-1-yl)-8-methyl-pyrido[2,3-d]pyrimidin-7-one (120 mg, 246.1 μmol, 1 eq) in THF (1 mL)/H₂O (1 mL) was added NaHCO₃ (62.0 mg, 738.4 μmol, 28.7 L, 3 eq) and prop-2-enoyl chloride (22.3 mg, 246.1 μmol, 20.0 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 15%-45% B over 8.0 min) to give 2-[4-[2-(dimethylamino)ethoxy]anilino]-6-(7-hydroxy-4-prop-2-enoyl-2,3-dihydroquinoxalin-1-yl)-8-methyl-pyrido[2,3-d]pyrimidin-7-one (89.3 mg, 136.1 μmol, 55.3% yield, 100% purity, TFA) as a yellow solid.

### Procedure for Preparation of Compound 060

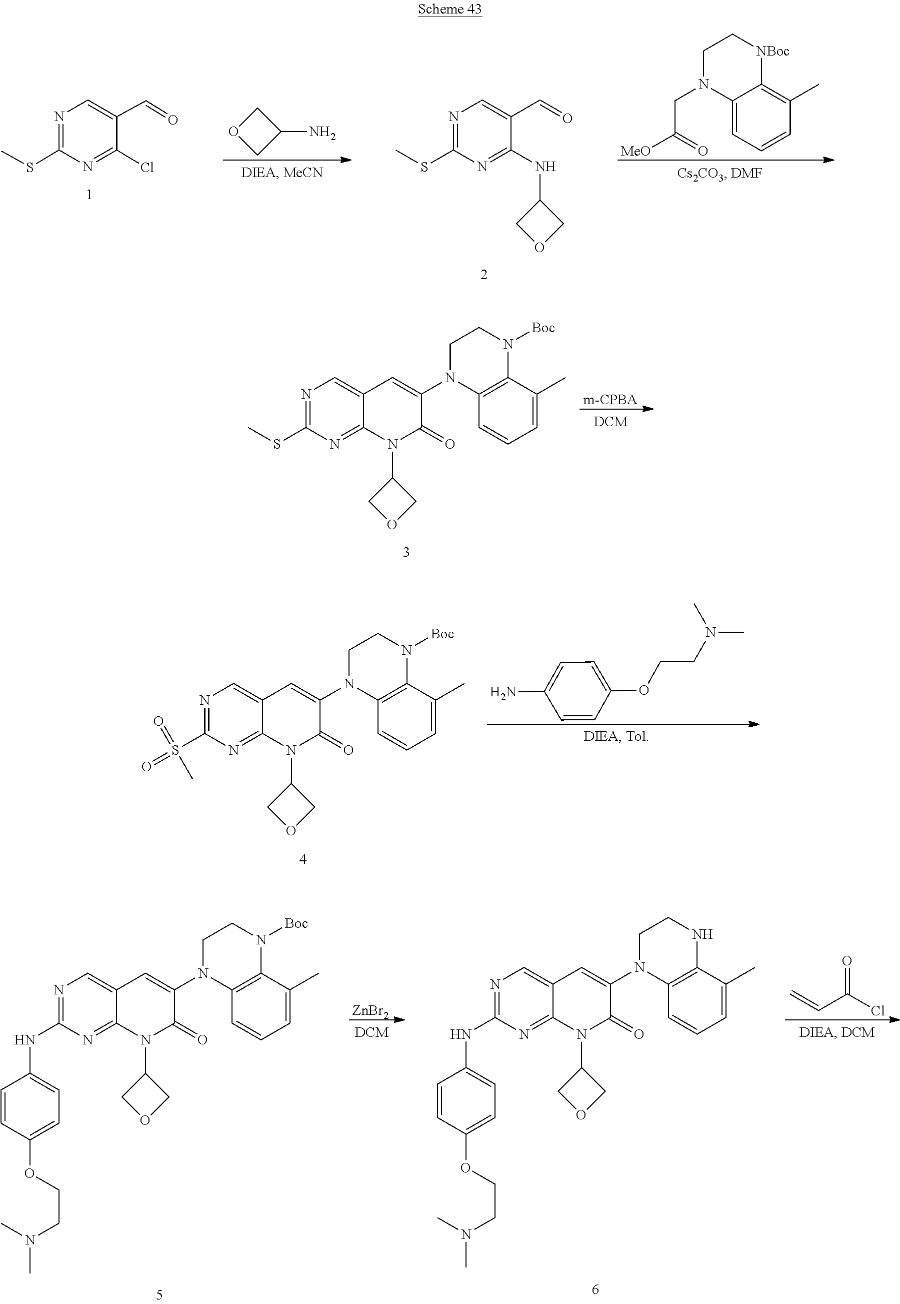

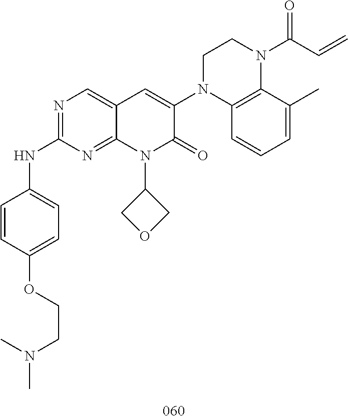 [SMILES: C=CC(=O)N1CCN(c2cc3cnc(Nc4ccc(OCCN(C)C)cc4)nc3n(C3COC3)c2=O)c2cccc(C)c21]

To a solution of oxetan-3-amine (774.9 mg, 10.6 mmol, 1 eq) and 4-chloro-2-methylsulfanyl-pyrimidine-5-carbaldehyde (2 g, 10.6 mmol, 1 eq) in MeCN (30 mL) was added DIEA (4.1 g, 31.8 mmol, 5.5 mL, 3 eq). The mixture was stirred at 25° C. for 2 hr. LC-MS showed desired compound was detected. The reaction mixture was concentrated under reduced pressure to give a residue. The residue was purified by flash silica gel chromatography (20 g Silica Flash Column, Eluent of 0˜10% Ethyl acetate/Petroleum ether gradient @ 100 mL/min) to give compound 2-methylsulfanyl-4-(oxetan-3-ylamino)pyrimidine-5-carbaldehyde (1 g, 4.4 mmol, 41.9% yield) as a yellow oil.

To a solution of 2-methylsulfanyl-4-(oxetan-3-ylamino)pyrimidine-5-carbaldehyde (300 mg, 1.3 mmol, 1 eq) and tert-butyl 4-(2-ethoxy-2-oxo-ethyl)-8-methyl-2,3-dihydroquinoxaline-1-carboxylate (445.4 mg, 1.3 mmol, 1 eq) in DMF (20 mL) was added Cs₂CO₃ (1.3 g, 4.0 mmol, 3 eq). The mixture was stirred at 60° C. for 12 hr. LC-MS showed desired compound was detected. The reaction mixture was concentrated under reduced pressure to give a residue. The residue was diluted with H₂O (20 mL) and extracted with ethyl acetate (30 mL×4). The combined organic layers were washed with brine (30 mL×3), dried over Na₂SO₄, filtered and concentrated under reduced pressure to give a residue. The residue was purified by flash silica gel chromatography (12 g Silica Flash Column, Eluent of 0˜50% Ethyl acetate/Petroleum ether gradient @ 60 mL/min) to give compound tert-butyl 8-methyl-4-[2-methylsulfanyl-8-(oxetan-3-yl)-7-oxo-pyrido[2,3-d]pyrimidin-6-yl]-2,3-dihydroquinoxaline-1-carboxylate (340 mg, 686.1 μmol, 51.5% yield) as a yellow solid.

To a solution of tert-butyl 8-methyl-4-[2-methylsulfanyl-8-(oxetan-3-yl)-7-oxo-pyrido[2,3-d]pyrimidin-6-yl]-2,3-dihydroquinoxaline-1-carboxylate (440 mg, 887.8 μmol, 1 eq) in DCM (5 mL) was added m-CPBA (216.3 mg, 1.1 mmol, 85% purity, 1·2 eq). The mixture was stirred at 25° C. for 1 hr. LC-MS showed desired compound was detected. The reaction mixture was quenched by addition sat·Na₂SO₃ (20 mL) at 25° C. and extracted with dichloromethane (20 mL×3). The combined organic layers were washed with sat. NaHCO₃ (20 mL× 3), dried over Na₂SO₄, filtered and concentrated under reduced pressure to give compound tert-butyl 8-methyl-4-[2-methylsulfonyl-8-(oxetan-3-yl)-7-oxo-pyrido[2,3-d]pyrimidin-6-yl]-2,3-dihydroquinoxaline-1-carboxylate (550 mg, crude) as an orange solid.

To a solution of tert-butyl 8-methyl-4-[2-methylsulfonyl-8-(oxetan-3-yl)-7-oxo-pyrido[2,3-d]pyrimidin-6-yl]-2,3-dihydroquinoxaline-1-carboxylate (512.5 mg, 971.5 μmol, 1 eq) in Tol. (10 mL) was added 4-[2-(dimethylamino)ethoxy]aniline (262.7 mg, 1.5 mmol, 1.5 eq) and DIEA (627.8 mg, 4.9 mmol, 846.1 μL, 5 eq). The mixture was stirred at 100° C. for 12 hr. LC-MS showed desired compound was detected. The reaction was concentrated under reduce pressure to give a residue. The residue was purified by prep-HPLC (FA condition; column: Phenomenex Luna C18 100×30 mm× 5 μm; mobile phase: [H₂O (0·2% FA)-ACN]; gradient: 20%-60% B over 8.0 min) to give compound tert-butyl 4-[2-[4-[2-(dimethylamino)ethoxy]anilino]-8-(oxetan-3-yl)-7-oxo-pyrido[2,3-d]pyrimidin-6-yl]-8-methyl-2,3-dihydroquinoxaline-1-carboxylate (130 mg, 207.1 μmol, 21.3% yield) as a yellow solid.

To a solution of tert-butyl 4-[2-[4-[2-(dimethylamino)ethoxy]anilino]-8-(oxetan-3-yl)-7-oxo-pyrido[2,3-d]pyrimidin-6-yl]-8-methyl-2,3-dihydroquinoxaline-1-carboxylate (119 mg, 189.6 μmol, 1 eq) in DCM (1 mL) was added ZnBr₂ (853.8 mg, 3.8 mmol, 189.7 μL, 20 eq). The mixture was stirred at 25° C. for 18 hr. LC-MS showed desired compound was detected. The reaction was concentrated under reduce pressure to give a residue. The residue was purified by prep-HPLC (FA condition; column: Phenomenex Luna C18 100×30 mm× 5 μm; mobile phase: [H₂O (0·2% FA)-ACN]; gradient: 5%-35% B over 8.0 min) to give compound 2-[4-[2-(dimethylamino)ethoxy]anilino]-6-(5-methyl-3,4-dihydro-2H-quinoxalin-1-yl)-8-(oxetan-3-yl)pyrido[2,3-d]pyrimidin-7-one (30 mg, 50.6 μmol, 26.7% yield, 88.9% purity) as a yellow solid.

To a solution of 2-[4-[2-(dimethylamino)ethoxy]anilino]-6-(5-methyl-3,4-dihydro-2H-quinoxalin-1-yl)-8-(oxetan-3-yl)pyrido[2,3-d]pyrimidin-7-one (23 mg, 40.1 μmol, 1 eq, FA) in DCM (1 mL) was added DIEA to adjust the pH to 7, then DIEA (15.5 mg, 120.3 μmol, 20.9 μL, 3 eq) and prop-2-enoyl chloride (3.6 mg, 40.1 μmol, 3.3 μL, 1 eq) was added to the mixture at 0° C. The mixture was stirred at 0° C. for 1 hr. LC-MS showed desired compound was detected. The reaction was concentrated under reduce pressure to give a residue. The residue was purified by prep-HPLC (FA condition; column: Phenomenex Luna C18 100×30 mm×5 μm; mobile phase: [H₂O (0·2% FA)-ACN]; gradient: 5%-45% B over 8.0 min) to give compound 2-[4-[2-(dimethylamino)ethoxy]anilino]-6-(5-methyl-4-prop-2-enoyl-2,3-dihydroquinoxalin-1-yl)-8-(oxetan-3-yl)pyrido[2,3-d]pyrimidin-7-one (3.9 mg, 6.5 μmol, 8.0% yield, 94.3% purity) as a

### Procedure for Preparation of Compound 079

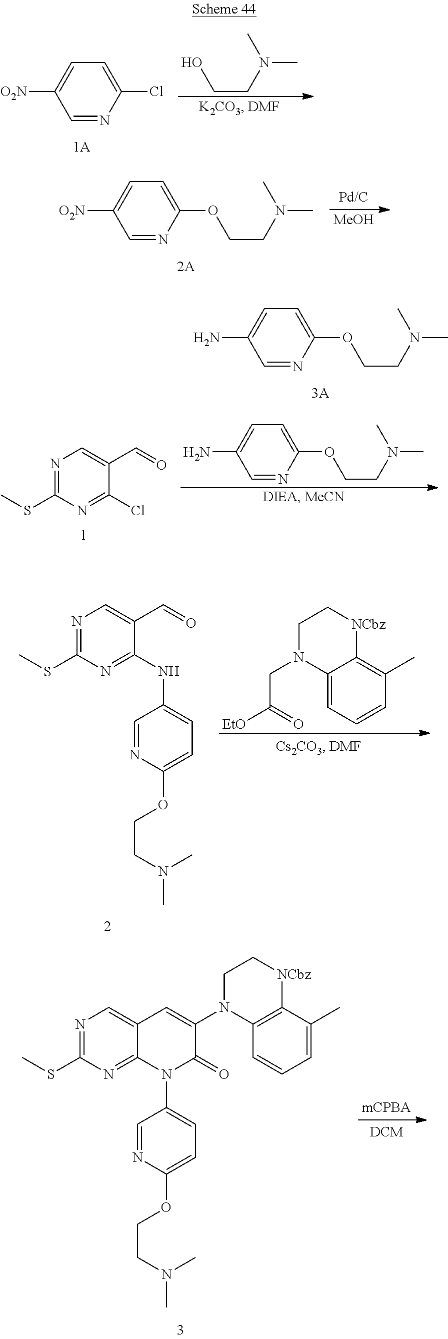

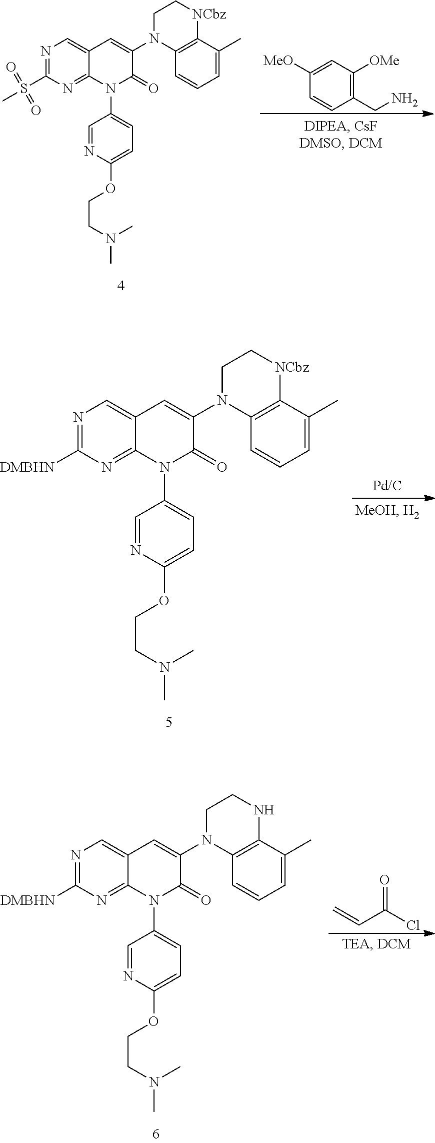

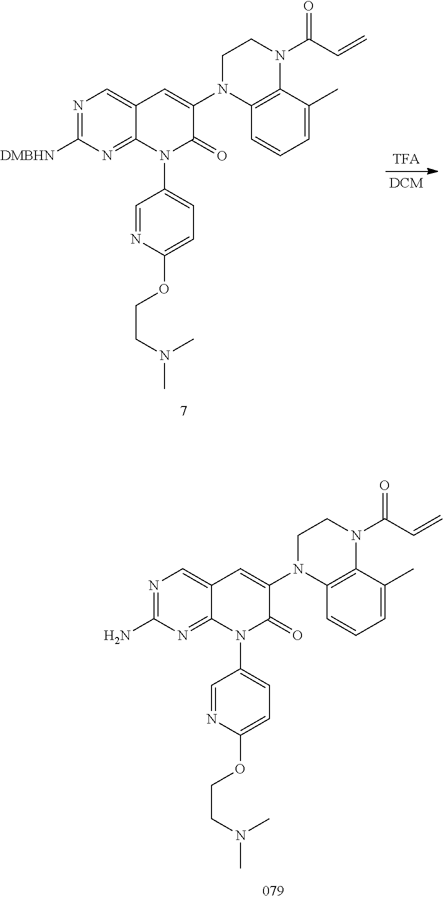

To a solution of 2-chloro-5-nitro-pyridine (0.5 g, 3·2 mmol, 1 eq) in DMF (10 mL) was added 2-(dimethylamino)ethanol (337.3 mg, 3.8 mmol, 379.9 μL, 1·2 eq) and K₂CO₃ (1.3 g, 9.5 mmol, 3 eq). The mixture was stirred at 60° C. for 2 hr (batch×2). LC-MS showed desired compound was detected. The residue was diluted with H₂O (40 mL) and extracted with ethyl acetate (40 mL× 3). The combined organic layers were washed with aqueous NaCl (20 ml×3), dried over Na₂SO₄, filtered and concentrated under reduced pressure to give a residue. The residue was purified by flash silica gel chromatography (20 g Silica Flash Column, Eluent of 0˜67% Ethyl acetate/Petroleum ether gradient @ 120 mL/min) to give N,N-dimethyl-2-[(5-nitro-2-pyridyl)oxy]ethanamine (1·2 g, 5.4 mmol, 85.6% yield) as a black oil.

To a solution of Pd/C (200 mg, 10% purity) in MeOH (10 mL) was added N, N-dimethyl-2-[(5-nitro-2-pyridyl)oxy]ethanamine (600 mg, 2.8 mmol, 1 eq) under N₂ atmosphere. The suspension was degassed and purged with H₂ for 3 times. The mixture was stirred under H₂ (15 Psi) at 20° C. for 3 hr. LC-MS showed desired compound was detected. The residue was filtered and concentrated under reduced pressure to give 2 6-[2-(dimethylamino)ethoxy]pyridin-3-amine (520 mg, crude) as a green oil.

To a solution of 6-[2-(dimethylamino)ethoxy]pyridin-3-amine (500 mg, 2.8 mmol, 1 eq) in MeCN (5 mL) was added DIEA (1.1 g, 8.3 mmol, 1.4 mL, 3 eq) and 4-chloro-2-methylsulfanyl-pyrimidine-5-carbaldehyde (520.0 mg, 2.8 mmol, 1 eq). The mixture was stirred at 20° C. for 3.5 hr. LC-MS showed desired compound was detected. The reaction mixture was concentrated under reduced pressure to remove solvent. The residue was purified by flash silica gel chromatography (12 g Silica Flash Column, Eluent of 0˜10% Dichloromethane/Methanol ether gradient @ 120 mL/min) to give 4-[[6-[2-(dimethylamino)ethoxy]-3-pyridyl]amino]-2-methylsulfanyl-pyrimidine-5-carbaldehyde (1 g) as a yellow solid.

To a solution of 4-[[6-[2-(dimethylamino)ethoxy]-3-pyridyl]amino]-2-methylsulfanyl-pyrimidine-5-carbaldehyde (500 mg, 1.5 mmol, 1 eq) in DMF (5 mL) was added benzyl 4-(2-ethoxy-2-oxo-ethyl)-8-methyl-2,3-dihydroquinoxaline-1-carboxylate (552.5 mg, 1.5 mmol, 1 eq) and Cs₂CO₃ (1.5 g, 4.5 mmol, 3 eq). The mixture was stirred at 60° C. for 12 hr. LC-MS showed desired compound was detected. The residue was diluted with H₂O (50 mL) and extracted with ethyl acetate (50 mL× 3). The combined organic layers were washed with aqueous NaCl (20 mL× 2), dried over Na₂SO₄, filtered and concentrated under reduced pressure to give a residue. The residue was purified by flash silica gel chromatography (12 g Silica Flash Column, Eluent of 0˜100% Ethyl acetate/Petroleum ether gradient @ 100 mL/min) to give compound benzyl 4-[8-[6-[2-(dimethylamino)ethoxy]-3-pyridyl]-2-methylsulfanyl-7-oxo-pyrido[2,3-d]pyrimidin-6-yl]-8-methyl-2,3-dihydroquinoxaline-1-carboxylate (500 mg, 784.0 μmol, 52.3% yield) as a yellow oil.

To a solution of benzyl 4-[8-[6-[2-(dimethylamino)ethoxy]-3-pyridyl]-2-methylsulfanyl-7-oxo-pyrido[2,3-d]pyrimidin-6-yl]-8-methyl-2,3-dihydroquinoxaline-1-carboxylate (700 mg, 1.1 mmol, 1 eq) in DCM (10 mL) was added mCPBA (891.3 mg, 4.4 mmol, 85% purity, 4 eq) at 0° C. The mixture was stirred at 20° C. for 12 hr. LC-MS showed desired compound was detected. The residue was diluted with Na₂SO₃ (250 mL) and extracted with ethyl acetate (200 mL×5). The combined organic layers were washed with NaHCO₃ (100 mL× 2), dried over Na₂SO₄, filtered and concentrated under reduced pressure to give benzyl 4-[8-[6-[2-(dimethylamino)ethoxy]-3-pyridyl]-2-methylsulfonyl-7-oxo-pyrido[2,3-d]pyrimidin-6-yl]-8-methyl-2,3-dihydroquinoxaline-1-carboxylate (360 mg, crude) as a yellow solid.

To a solution of benzyl 4-[8-[6-[2-(dimethylamino)ethoxy]-3-pyridyl]-2-methylsulfonyl-7-oxo-pyrido[2,3-d]pyrimidin-6-yl]-8-methyl-2,3-dihydroquinoxaline-1-carboxylate (330 mg, 492.7 μmol, 1 eq) and (2,4-dimethoxyphenyl) methanamine (98.9 mg, 591.3 μmol, 88.8 μL, 1·2 eq) in DMSO (4 mL) and DCM (4 mL) was added DIEA (95.5 mg, 739.1 μmol, 128.7 μL, 1.5 eq) and CsF (224.5 mg, 1.5 mmol, 54.6 μL, 3 eq). The mixture was stirred at 25° C. for 2 hr. LC-MS showed desired compound was detected. The reaction mixture was concentrated under reduced pressure to give a residue. The residue was purified by prep-HPLC (TFA condition column: Phenomenex Luna C18 75×30 mm×3 μm; mobile phase: [H₂O (0.1% TFA)-ACN]; gradient: 30%-60% B over 8.0 min) to give benzyl 4-[2-[(2,4-dimethoxyphenyl)methylamino]-8-[6-[2-(dimethylamino)ethoxy]-3-pyridyl]-7-oxo-pyrido[2,3-d]pyrimidin-6-yl]-8-methyl-2,3-dihydroquinoxaline-1-carboxylate (210 mg, 277.5 μmol, 56.3% yield) as a light yellow solid.

To a solution of Pd/C (100 mg, 10% purity) in MeOH (5 mL) was added benzyl 4-[2-[(2,4-dimethoxyphenyl)methylamino]-8-[6-[2-(dimethylamino)ethoxy]-3-pyridyl]-7-oxo-pyrido[2,3-d]pyrimidin-6-yl]-8-methyl-2,3-dihydroquinoxaline-1-carboxylate (120 mg, 158.6 μmol, 1 eq) and TEA (48.1 mg, 475.7 μmol, 66·2 μL, 3 eq) under N₂ atmosphere. The suspension was degassed and purged with H₂ for 3 times. The mixture was stirred under H₂ (15 Psi) at 20° C. for 2 hr. LC-MS showed desired compound was detected. The reaction mixture was filtered and concentrated under reduced pressure to give a 2-[(2,4-dimethoxyphenyl)methylamino]-8-[6-[2-(dimethylamino)ethoxy]-3-pyridyl]-6-(5-methyl-3,4-dihydro-2H-quinoxalin-1-yl)pyrido[2,3-d]pyrimidin-7-one (86 mg, crude) as a brown oil.

To a solution of 2-[(2,4-dimethoxyphenyl)methylamino]-8-[6-[2-(dimethylamino)ethoxy]-3-pyridyl]-6-(5-methyl-3,4-dihydro-2H-quinoxalin-1-yl)pyrido[2,3-d]pyrimidin-7-one (86 mg, 138.1 μmol, 1 eq) in DCM (1 mL) was added TEA (41.9 mg, 414.3 μmol, 57.7 L, 3 eq) and prop-2-enoyl chloride (3.5 mg, 38.5 μmol, 3.1 μL, 1 eq) at 0° C. The mixture was stirred at 0° C. for 1 hr. LC-MS showed desired compound was detected. The reaction mixture was concentrated under reduced pressure to give 2-[(2,4-dimethoxyphenyl)methylamino]-8-[6-[2-(dimethylamino)ethoxy]-3-pyridyl]-6-(5-methyl-4-prop-2-enoyl-2,3-dihydroquinoxalin-1-yl)pyrido[2,3-d]pyrimidin-7-one (80 mg, crude) as a brown oil.

To a solution of 2-[(2,4-dimethoxyphenyl)methylamino]-8-[6-[2-(dimethylamino)ethoxy]-3-pyridyl]-6-(5-methyl-4-prop-2-enoyl-2,3-dihydroquinoxalin-1-yl)pyrido[2,3-d]pyrimidin-7-one (80 mg, 118·2 μmol, 1 eq) in DCM (1 mL) was added TFA (134.8 mg, 1·2 mmol, 87.8 μL, 10 eq). The mixture was stirred at 20° C. for 3 hr. LC-MS showed desired compound was detected. The reaction mixture was concentrated under reduced pressure to give a residue. The residue was purified by prep-HPLC (TFA condition column: Phenomenex Luna C18 75×30 mm×3 μm; mobile phase: [H₂O (0.1% TFA)-ACN]; gradient: 10%-40% B over 8.0 min) to give 2-amino-8-[6-[2-(dimethylamino)ethoxy]-3-pyridyl]-6-(5-methyl-4-prop-2-enoyl-2,3-dihydroquinoxalin-1-yl)pyrido[2,3-d]pyrimidin-7-one (9 mg, 13.8 μmol, 11.6% yield, 98.0% purity, TFA) as a green

### Procedure for Preparation of Compound 080

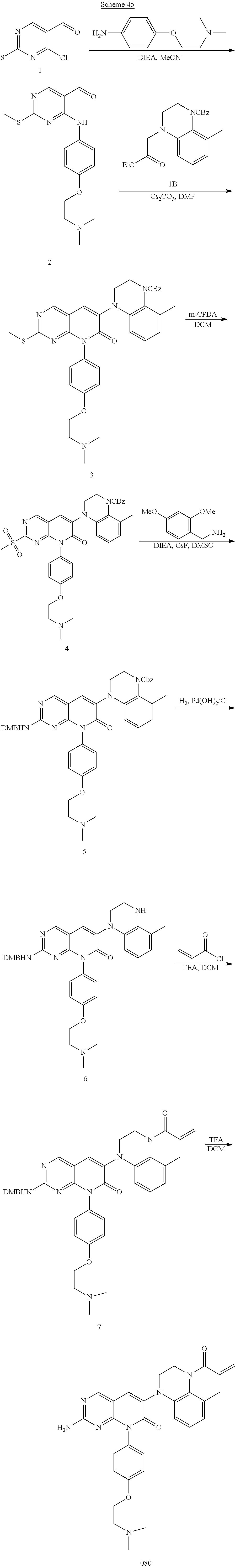

To a solution of 4-chloro-2-methylsulfanyl-pyrimidine-5-carbaldehyde (500 mg, 2.6 mmol, 1 eq) in MeCN (10 mL) was added DIEA (1 g, 7.9 mmol, 1.3 mL, 3 eq) to adjust pH=8-9 and 4-[2-(dimethylamino)ethoxy]aniline (477.7 mg, 2.6 mmol, 1 eq). The mixture was stirred at 20° C. for 4 hr. LC-MS showed desired compound was detected. The reaction mixture was concentrated under reduced pressure to remove solvent. The crude product was triturated with ethyl acetate (5 mL) at 20° C. for 12 hr to give 4-[4-[2-(dimethylamino)ethoxy]anilino]-2-methylsulfanyl-pyrimidine-5-carbaldehyde (890 mg, 2.3 mmol, 89.9% yield, 89% purity) as a brown solid.

To a solution of 4-[4-[2-(dimethylamino)ethoxy]anilino]-2-methylsulfanyl-pyrimidine-5-carbaldehyde (800 mg, 2.4 mmol, 1 eq) in DMF (8 mL) was added benzyl 4-(2-ethoxy-2-oxo-ethyl)-8-methyl-2,3-dihydroquinoxaline-1-carboxylate (886.6 mg, 2.4 mmol, 1 eq) and Cs₂CO₃ (2.3 g, 7·2 mmol, 3 eq). The mixture was stirred at 60° C. for 2 hr. LC-MS showed desired compound was detected. The reaction mixture was extracted with H₂O (20 mL) and ethyl acetate (20 mL× 3). The combined organic layers were washed with brine (20 mL× 3), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 100 mL/min) to give benzyl 4-[8-[4-[2-(dimethylamino)ethoxy]phenyl]-2-methylsulfanyl-7-oxo-pyrido[2,3-d]pyrimidin-6-yl]-8-methyl-2,3-dihydroquinoxaline-1-carboxylate (840 mg, 1·2 mmol, 52% yield, 95% purity) as a yellow solid.

At 0° C., to a solution of benzyl 4-[8-[4-[2-(dimethylamino)ethoxy]phenyl]-2-methylsulfanyl-7-oxo-pyrido[2,3-d]pyrimidin-6-yl]-8-methyl-2,3-dihydroquinoxaline-1-carboxylate (840 mg, 1.3 mmol, 1 eq) in DCM (10 mL) was added m-CPBA (803.4 mg, 3.9 mmol, 85% purity, 3 eq). The mixture was stirred at 20° C. for 2 hr. LC-MS showed desired compound was detected. The reaction mixture was quenched by addition Na₂SO₃ (50 mL) and extracted with dichloromethane (20 mL× 3). The combined organic layers were washed with NaHCO₃ (20 mL× 3), dried over Na₂SO₄, filtered and concentrated under reduced pressure to give benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (1 g, crude) as an orange solid.

To a solution of benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (850 mg, 1·2 mmol, 1 eq) in DMSO (10 mL) was added (2,4-dimethoxyphenyl) methanamine (255 mg, 1.5 mmol, 229.1 μL, 1·2 eq), DIEA (492.8 mg, 3.8 mmol, 664.1 μL, 3 eq) and CsF (289.6 mg, 1.9 mmol, 70.3 μL, 1.5 eq). The mixture was stirred at 60° C. for 1 hr. LC-MS showed desired compound was detected. The residue was extracted with dichloromethane:methanol=10:1 (20 mL× 3), dried over Na₂SO₄, filtered and concentrated under reduced pressure to give a residue. The residue was purified by prep-HPLC (TFA condition; column: Phenomenex luna C18 100×40 mm× 5 μm; mobile phase: [H₂O (0.1% TFA)-ACN]; gradient: 30%-60% B over 8.0 min) to give benzyl 4-[2-[(2,4-dimethoxyphenyl)methylamino]-8-[4-[2-(dimethylamino)ethoxy]phenyl]-7-oxopyrido[2,3-d]pyrimidin-6-yl]-8-methyl-2,3-dihydroquinoxaline-1-carboxylate (145 mg, 191.8 μmol, 15% yield) as a red solid.

To a solution of benzyl 4-[2-[(2,4-dimethoxyphenyl)methylamino]-8-[4-[2-(dimethylamino)ethoxy]phenyl]-7-oxo-pyrido[2,3-d]pyrimidin-6-yl]-8-methyl-2,3-dihydroquinoxaline-1-carboxylate (129 mg, 170.6 μmol, 1 eq) in MeOH (5 mL) was added Pd(OH)₂/C (70 mg, 20% purity) under N₂ atmosphere. The suspension was degassed and purged with H₂ for 3 times. The mixture was stirred under H₂ (15 Psi.) at 20° C. for 12 hr. LC-MS showed desired compound was detected. The reaction mixture was filtered and concentrated under reduced pressure to remove solvent. The residue was purified by prep-HPLC (TFA condition, column: Phenomenex luna C18 80×30 mm×3 μm; mobile phase: [H₂O (0.1% TFA)-ACN]; gradient: 5%-45% B over 8.0 min) to give 2-[(2,4-dimethoxyphenyl)methylamino]-8-[4-[2-(dimethylamino)ethoxy]phenyl]-6-(5-methyl-3,4-dihydro-2H-quinoxalin-1-yl)pyrido[2,3-d]pyrimidin-7-one (18 mg, 26.3 μmol, 15.4% yield, 91% purity) as a yellow solid.

To a solution of 2-[(2,4-dimethoxyphenyl)methylamino]-8-[4-[2-(dimethylamino)ethoxy]phenyl]-6-(5-methyl-3,4-dihydro-2H-quinoxalin-1-yl)pyrido[2,3-d]pyrimidin-7-one (18 mg, 28.9 μmol, 1 eq) in DCM (1 mL) was added dropwise TEA (8.7 mg, 86.8 μmol, 12 μL, 3 eq) and then prop-2-enoyl chloride (2.6 mg, 28.9 μmol, 2.3 μL, 1 eq) in DCM (0.1 mL) was added dropwise at 0° C. The mixture was stirred at 0° C. for 30 min. LC-MS showed desired compound was detected. The reaction mixture was concentrated under N₂ to give 2-[(2,4-dimethoxyphenyl)methylamino]-8-[4-[2-(dimethylamino)ethoxy]phenyl]-6-(5-methyl-4-prop-2-enoyl-2,3-dihydroquinoxalin-1-yl)pyrido[2,3-d]pyrimidin-7-one (40 mg, crude) as a yellow solid.

To a solution of 2-[(2,4-dimethoxyphenyl)methylamino]-8-[4-[2-(dimethylamino)ethoxy]phenyl]-6-(5-methyl-4-prop-2-enoyl-2,3-dihydroquinoxalin-1-yl)pyrido[2,3-d]pyrimidin-7-one (40 mg, 59.1 μmol, 1 eq) in DCM (1 mL) was added TFA (460.5 mg, 4 mmol, 0.3 mL, 68·2 eq). The mixture was stirred at 20° C. for 12 hr. LC-MS showed desired compound was detected. The reaction mixture was concentrated under reduced pressure to remove solvent. The residue was purified by prep-HPLC (TFA condition, column: Phenomenex luna C18 100×40 mm×5 μm; mobile phase: [H₂O (0.1% TFA)-ACN]; gradient: 1%-35% B over 8.0 min) to give 2-amino-8-[4-[2-(dimethylamino)ethoxy]phenyl]-6-(5-methyl-4-prop-2-enoyl-2,3-dihydroquinoxalin-1-yl)pyrido[2,3-d]pyrimidin-7-one (7.5 mg, 13.7 μmol, 23.1% yield, 96% purity) as a yellow solid.

### Procedure for Preparation of Compound 081

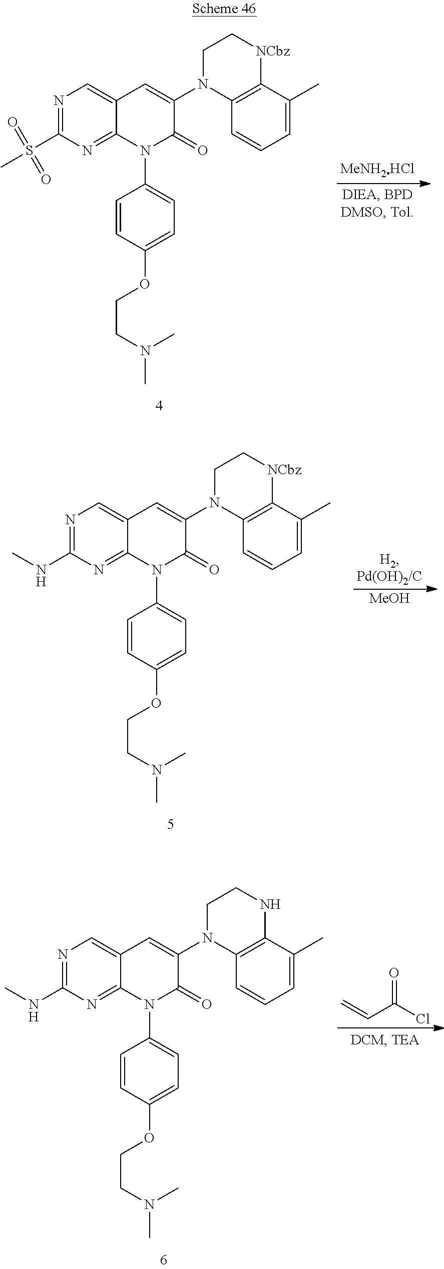

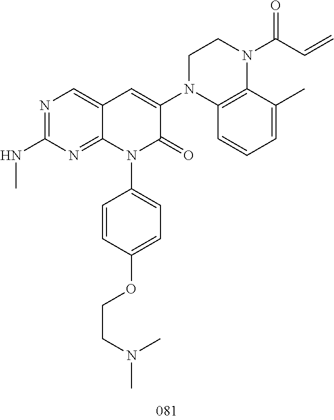 [SMILES: C=CC(=O)N1CCN(c2cc3cnc(NC)nc3n(-c3ccc(OCCN(C)C)cc3)c2=O)c2cccc(C)c21]

To a solution of benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (170 mg, 254.2 μmol, 1 eq) in Tol. (2 mL) and DMSO (0.1 mL) was added MeNH₂. HCl (25.7 mg, 381.3 μmol, 1.5 eq) and DIEA (164.3 mg, 1.3 mmol, 221.4 μL, 5 eq). The mixture was stirred at 25° C. for 2 hr. Then BPD (193.7 mg, 762.6 μmol, 3 eq) was added to the mixture. The mixture was stirred at 25° C. for 1 hr. LC-MS showed the desired compound was detected. The reaction mixture was concentrated under reduced pressure to remove solvent to get the residue was purified by prep-HPLC (TFA condition; column: Phenomenex Luna C18 75×30 mm×3 μm; mobile phase: [H₂O (0.1% TFA)-ACN]; gradient: 25%-55% B over 8.0 min) to give benzyl 4-[8-[4-[2-(dimethylamino)ethoxy]phenyl]-2-(methylamino)-7-oxo-pyrido[2,3-d]pyrimidin-6-yl]-8-methyl-2,3-dihydroquinoxaline-1-carboxylate (170 mg, 274.3 μmol, 107.9% yield) as a yellow

To a solution of Pd(OH)₂/C (150 mg, 20% purity) in MeOH (5 mL) was added benzyl 4-[8-[4-[2-(dimethylamino)ethoxy]phenyl]-2-(methylamino)-7-oxo-pyrido[2,3-d]pyrimidin-6-yl]-8-methyl-2,3-dihydroquinoxaline-1-carboxylate (110 mg, 177.5 μmol, 1 eq) under N₂ atmosphere. The suspension was degassed and purged with H₂ for 3 times. The mixture was stirred under H₂ (15 Psi) at 25° C. for 15 min. LC-MS showed the desired compound was detected. The reaction mixture filtered and concentrated under reduced pressure to give 8-[4-[2-(dimethylamino)ethoxy]phenyl]-2-(methylamino)-6-(5-methyl-3,4-dihydro-2H-quinoxalin-1-yl)pyrido[2,3-d]pyrimidin-7-one (70 mg, crude) as a yellow solid.

To a solution of 8-[4-[2-(dimethylamino)ethoxy]phenyl]-2-(methylamino)-6-(5-methyl-3,4-dihydro-2H-quinoxalin-1-yl)pyrido[2,3-d]pyrimidin-7-one (70 mg, 144·2 μmol, 1 eq) and TEA (43.8 mg, 432.5 μmol, 60·2 μL, 3 eq) in DCM (1 mL) was added prop-2-enoyl chloride (13.1 mg, 144·2 μmol, 11.7 μL, 1 eq) at 0° C. The mixture was stirred at 0° C. for 1 hr. LC-MS showed the desired compound was detected. The reaction mixture was concentrated under reduced pressure to remove solvent to get the residue. The residue was purified by prep-HPLC (TFA condition; column: Phenomenex luna C18 100×40 mm× 3 μm; mobile phase: [H₂O (0.1% TFA)-ACN]; gradient: 10%-40% B over 8.0 min) to give 8-[4-[2-(dimethylamino)ethoxy]phenyl]-2-(methylamino)-6-(5-methyl-4-prop-2-enoyl-2,3-dihydroquinoxalin-1-yl)pyrido[2,3-d]pyrimidin-7-one (13.7 mg, 24.1 μmol, 16.7% yield, 95% purity) as a yellow solid.

### Procedure for Preparation of Compound 082

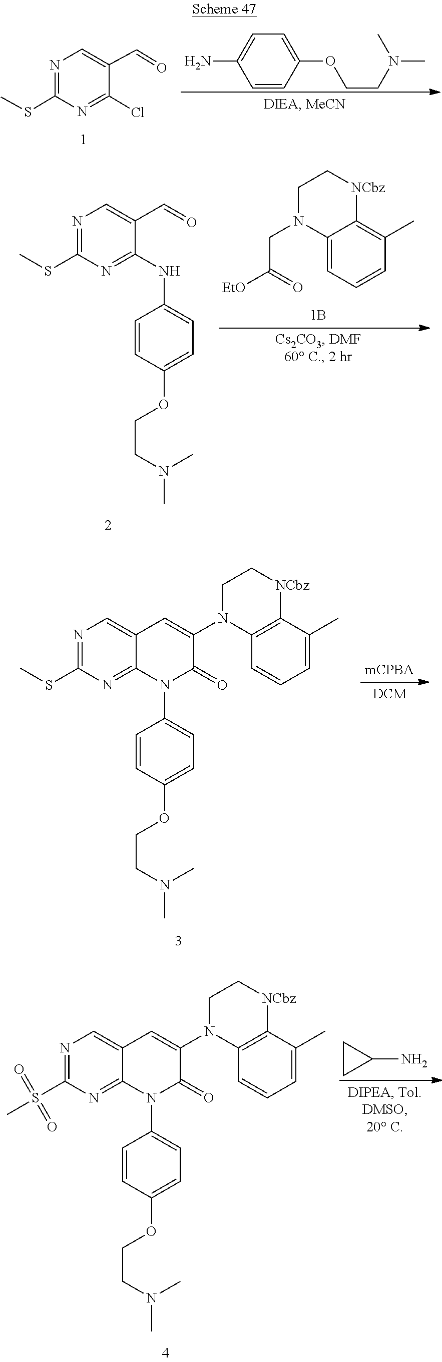

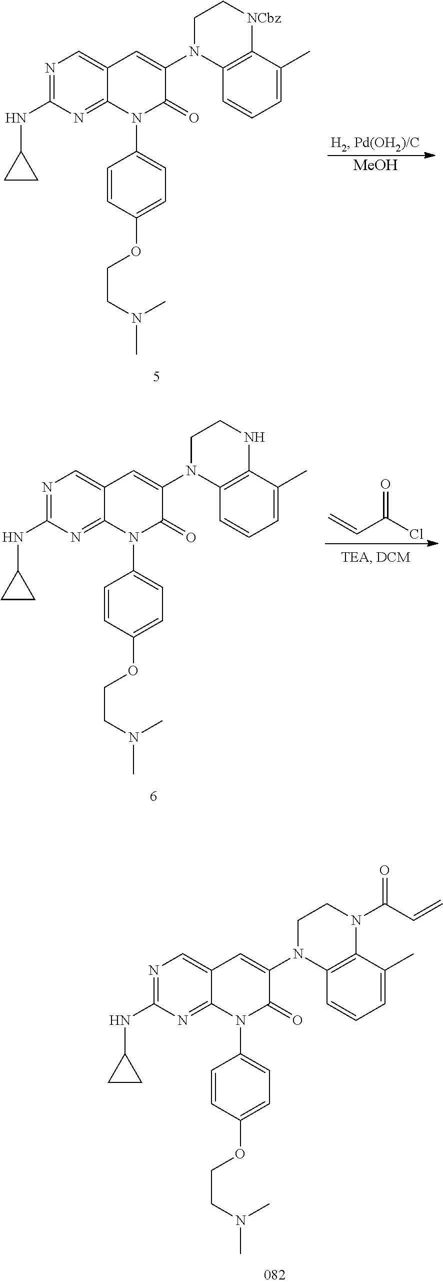

To a solution of 4-chloro-2-methylsulfanyl-pyrimidine-5-carbaldehyde (2 g, 10.6 mmol, 1 eq) in MeCN (40 mL) was added DIEA (4.1 g, 31.8 mmol, 5.5 mL, 3 eq) to adjust pH=8-9 and 4-[2-(dimethylamino)ethoxy]aniline (1.9 g, 10.6 mmol, 1 eq). The mixture was stirred at 20° C. for 12 hr. LC-MS showed desired compound was detected. The reaction mixture was concentrated under reduced pressure to remove solvent. The crude product was triturated with ethyl acetate (20 mL) at 20° C. for 12 hr to give 4-[4-[2-(dimethylamino)ethoxy]anilino]-2-methylsulfanyl-pyrimidine-5-carbaldehyde (2.4 g, 7·2 mmol, 68.1% yield) as a yellow solid.

To a solution of 4-[4-[2-(dimethylamino)ethoxy]anilino]-2-methylsulfanyl-pyrimidine-5-carbaldehyde (1 g, 3.0 mmol, 1 eq) in DMF (10 mL) was added Cs₂CO₃ (2.9 g, 9.0 mmol, 3 eq) and benzyl 4-(2-ethoxy-2-oxo-ethyl)-8-methyl-2,3-dihydroquinoxaline-1-carboxylate (1.1 g, 3.0 mmol, 1 eq). The mixture was stirred at 60° C. for 2 hr. LC-MS showed desired compound was detected. The residue was diluted with H₂O (50 mL) and extracted with ethyl acetate (50 mL×3). The combined organic layers were washed with brine (20 mL× 2), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @150 mL/min) to give benzyl 4-[8-[4-[2-(dimethylamino)ethoxy]phenyl]-2-methylsulfanyl-7-oxo-pyrido[2,3-d]pyrimidin-6-yl]-8-methyl-2,3-dihydroquinoxaline-1-carboxylate (990 mg, 1.6 mmol, 51.7% yield) obtained as a yellow solid.

To a solution of benzyl 4-[8-[4-[2-(dimethylamino)ethoxy]phenyl]-2-methylsulfanyl-7-oxo-pyrido[2,3-d]pyrimidin-6-yl]-8-methyl-2,3-dihydroquinoxaline-1-carboxylate (410 mg, 643.8 μmol, 1 eq) in DCM (20 mL) was added m-CPBA (235.3 mg, 1·2 mmol, 85% purity, 1.8 eq) at 0° C. The mixture was stirred at 0° C. for 2 hr. LC-MS showed desired compound was detected. The residue was diluted with Na₂SO₃ (50 mL) and extracted with DCM (50 mL× 3). The combined organic layers were washed with NaHCO₃ (30 mL×1), dried over Na₂SO₄, filtered and concentrated under reduced pressure to give benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (330 mg, crude) as a yellow solid.

To a solution of benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (200 mg, 299.1 μmol, 1 eq) in Tol. (2 mL) was added DIPEA (193.3 mg, 1.5 mmol, 260.4 μL, 5 eq) and cyclopropanamine (25.6 mg, 448.6 μmol, 31.1 μL, 1.5 eq). The mixture was stirred at 20° C. for 12 hr. (batch× 2) LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min) to give benzyl 4-[2-(cyclopropylamino)-8-[4-[2-(dimethylamino)ethoxy]phenyl]-7-oxo-pyrido[2,3-d]pyrimidin-6-yl]-8-methyl-2,3-dihydroquinoxaline-1-carboxylate (240 mg, 371.7 μmol, 62.1% yield) as a yellow solid.

To a solution of Pd(OH)₂/C (20 mg, 20% purity) in MeOH (2 mL) was added benzyl 4-[2-(cyclopropylamino)-8-[4-[2-(dimethylamino)ethoxy]phenyl]-7-oxo-pyrido[2,3-d]pyrimidin-6-yl]-8-methyl-2,3-dihydroquinoxaline-1-carboxylate (200 mg, 309.7 μmol, 1 eq) under N₂ atmosphere. The suspension was degassed and purged with H₂ for 3 times. The mixture was stirred under H₂ (15 Psi) at 20° C. for 1 hr. LC-MS showed desired compound was detected. The reaction mixture was filtered and concentrated under reduced pressure to give 2-(cyclopropylamino)-8-[4-[2-(dimethylamino)ethoxy]phenyl]-6-(5-methyl-3,4-dihydro-2H-quinoxalin-1-yl)pyrido[2,3-d]pyrimidin-7-one (150 mg, crude) as a yellow solid.

To a solution of 2-(cyclopropylamino)-8-[4-[2-(dimethylamino)ethoxy]phenyl]-6-(5-methyl-3,4-dihydro-2H-quinoxalin-1-yl)pyrido[2,3-d]pyrimidin-7-one (100 mg, 195.5 μmol, 1 eq) in DCM (1 mL) was added TEA (59.3 mg, 586.4 μmol, 81.6 μL, 3 eq) and prop-2-enoyl chloride (17.7 mg, 195.5 μmol, 15.9 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition; column: Phenomenex luna C18 100×40 mm× 5 μm; mobile phase: [H₂O (0.1% TFA)-ACN]; gradient: 10%-50% B over 8.0 min) to give 2-(cyclopropylamino)-8-[4-[2-(dimethylamino)ethoxy]phenyl]-6-(5-methyl-4-prop-2-enoyl-2,3-dihydroquinoxalin-1-yl)pyrido[2,3-d]pyrimidin-7-one (37.7 mg, 61.4 μmol, 31.4% yield, 99.5% purity, FA) as a yellow solid.

### Procedure for Preparation of Compound 083

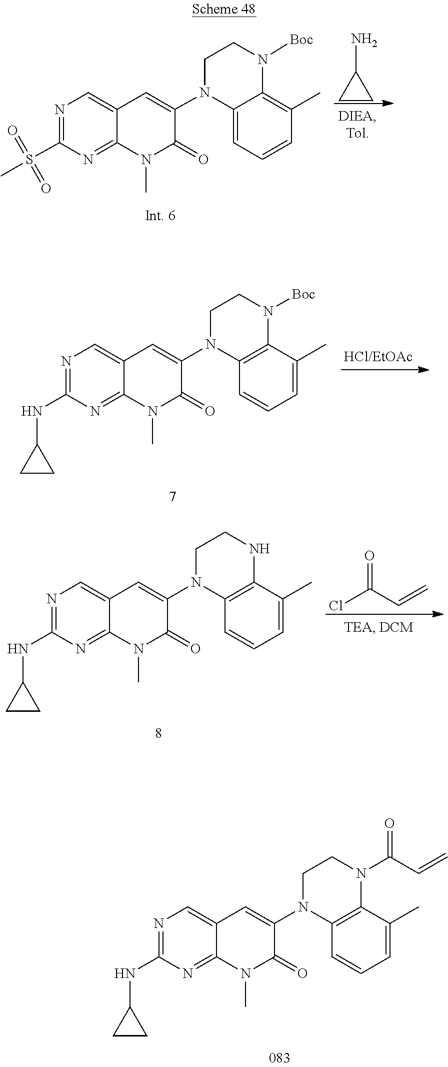

To a solution of tert-butyl 8-methyl-4-(8-methyl-2-methylsulfonyl-7-oxo-pyrido[2,3-d]pyrimidin-6-yl)-2,3-dihydroquinoxaline-1-carboxylate (200 mg, 411.9 μmol, 1 eq) and cyclopropanamine (35.3 mg, 617.9 μmol, 42.8 μL, 1.5 eq) in Tol. (2 mL) was added DIEA (266·2 mg, 2.1 mmol, 358.7 μL, 5 eq). The mixture was stirred at 25° C. for 12 hr. LC-MS showed the desired compound was detected. The reaction mixture was concentrated under reduced pressure to remove solvent. The residue was diluted with H₂O (50 mL) and extracted with ethyl acetate (50 mL×4). The combined organic layers were washed with brine (50 ml×1), dried over Na₂SO₄, filtered and concentrated under reduced pressure to give a residue. The residue was purified by flash silica gel chromatography (12 g Silica Flash Column, Eluent of 0˜50% Ethyl acetate/Petroleum ether gradient @ 60 mL/min) to give tert-butyl 4-[2-(cyclopropylamino)-8-methyl-7-oxo-pyrido[2,3-d]pyrimidin-6-yl]-8-methyl-2,3-dihydroquinoxaline-1-carboxylate (280 mg, 605.4 μmol, 73.5% yield) as a yellow solid.

A mixture of tert-butyl 4-[2-(cyclopropylamino)-8-methyl-7-oxo-pyrido[2,3-d]pyrimidin-6-yl]-8-methyl-2,3-dihydroquinoxaline-1-carboxylate (80 mg, 173.0 μmol, 1 eq) in EtOAc (1 mL) and

HCl/EtOAc (0.5 mL,4 M) was stirred at 25° C. for 1 hr. LC-MS showed the desired compound was detected. The reaction mixture was concentrated under reduced pressure to remove solvent t o give 2-(cyclopropylamino)-8-methyl-6-(5-methyl-3,4-dihydro-2H-quinoxalin-1-yl)pyrido[2,3-d]pyrimidin-7-one (62 mg, crude) as a brown solid.

To a solution of 2-(cyclopropylamino)-8-methyl-6-(5-methyl-3,4-dihydro-2H-quinoxalin-1-yl)pyrido[2,3-d]pyrimidin-7-one (100 mg, 275.9 μmol, 1 eq) and TEA (83.8 mg, 827.8 μmol, 115·2 μL, 3 eq) in DCM (1 mL) was added prop-2-enoyl chloride (25.0 mg, 275.9 μmol, 22.4 μL, 1 eq) at 0° C. The mixture was stirred at 0° C. for 1 hr. LC-MS showed the desired compound was detected. The reaction mixture was concentrated under reduced pressure to remove solvent. The residue was purified by prep-HPLC (TFA condition; column: Phenomenex Luna C18 75×30 mm× 3 μm; mobile phase: [H₂O (0.1% TFA)-ACN]; gradient: 25%-55% B over 8.0 min) to give 2-(cyclopropylamino)-8-methyl-6-(5-methyl-4-prop-2-enoyl-2,3-dihydroquinoxalin-1-yl)pyrido[2,3-d]pyrimidin-7-one (48.9 mg, 116·2 μmol, 42.1% yield, 99.3% purity) obtained as a

### Procedure for Preparation of Compound 085

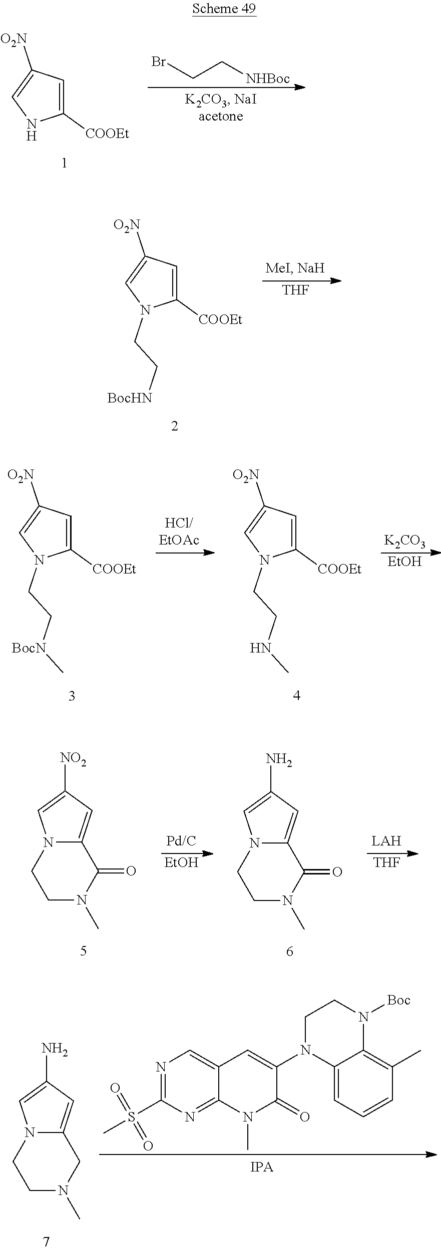

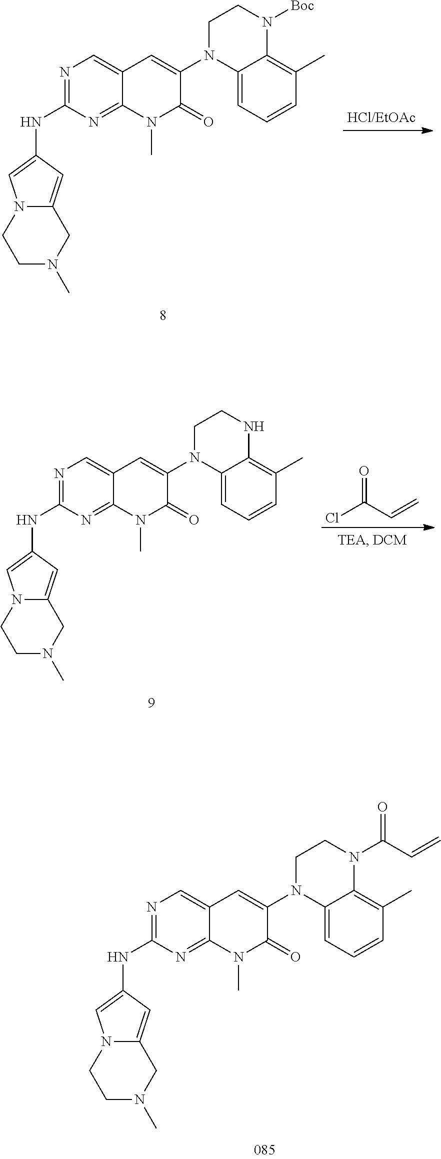

To a solution of ethyl 4-nitro-1H-pyrrole-2-carboxylate (1.5 g, 8·2 mmol, 1 eq) in acetone (80 mL) was added K₂CO₃ (2.3 g, 16.3 mmol, 2 eq) and NaI (1·2 g, 8·2 mmol, 1 eq) tert-butyl N-(2-bromoethyl) carbamate (2.7 g, 12·2 mmol, 1.5 eq). The mixture was stirred at 60° C. for 12 hr (2 batch worked together). LC-MS showed desired compound was detected. Water (20 mL) was added to the mixture and then extracted by ethyl acetate (20 mL×3), the combined organic layers was washed by brine (10 mL× 2), then dried by Na₂SO₄, filtered and the filtrate was concentrated to get the crude product ethyl 1-[2-(tert-butoxycarbonylamino)ethyl]-4-nitro-pyrrole-2-carboxylate (5 g, crude) as a brown solid used into the next step without further purification.

To a mixture of ethyl 1-[2-(tert-butoxycarbonylamino)ethyl]-4-nitro-pyrrole-2-carboxylate (2.5 g, 7.6 mmol, 1 eq) in THF (80 mL) was added NaH (366.6 mg, 9·2 mmol, 60% purity, 1·2 eq), the mixture was stirred at 25° C. for 20 min, then CH₃I (2.7 g, 19.1 mmol, 1·2 mL, 2.5 eq) was added, the mixture was stirred at 25° C. for 12 hr (2 batch worked together). LC-MS showed desired compound was detected. The mixture was quenched by sat·NH₄CI (10 mL) and then extracted by EtOAc (20 mL×3), the combined organic layers was washed by brine and then dried by Na₂SO₄, then filtered and then filtrate was concentrated to get the crude product ethyl 1-[2-[tert-butoxycarbonyl(methyl)amino]ethyl]-4-nitro-pyrrole-2-carboxylate (5 g, crude) as an orange solid used into the next step without further purification.

To a solution of ethyl 1-[2-[tert-butoxycarbonyl(methyl)amino]ethyl]-4-nitro-pyrrole-2-carboxylate (2.5 g, 7.3 mmol, 1 eq) in EtOAc (5 mL) was added HCl/EtOAc (10 mL) (4M). The mixture was stirred at 25° C. for 1 hr (2 batch worked together). LC-MS showed desired compound was detected. The mixture was concentrated to get the crude product ethyl 1-[2-(methylamino)ethyl]-4-nitro-pyrrole-2-carboxylate (4 g, crude, HCl) as a white solid used into the next step without further purification.

To a solution of ethyl 1-[2-(methylamino)ethyl]-4-nitro-pyrrole-2-carboxylate (2 g, 7·2 mmol, 1 eq, HCl) in EtOH (20 mL) was added K₂CO₃ (4.0 g, 28.8 mmol, 4 eq). The mixture was stirred at 80° C. for 12 hr (2 batch worked together). LC-MS showed desired compound was detected. The reaction mixture was concentrated under reduced pressure to remove solvent. The residue was diluted with water 5 mL and extracted with EtOAc (20 mL×3). The combined organic layers were dried over Na₂SO₄, filtered, and concentrated under reduced pressure to give a residue. The residue was triturated with petroleum ether/ethyl acetate=1/1, 20 mL at 25° C. for 12 hr to give compound 2-methyl-7-nitro-3,4-dihydropyrrolo[1,2-a]pyrazin-1-one (1.4 g, 7·2 mmol, 49.80% yield) as a yellow solid.

A mixture of 2-methyl-7-nitro-3,4-dihydropyrrolo[1,2-a]pyrazin-1-one (1.3 g, 6.7 mmol, 1 eq), Pd/C (300 mg, 10% purity) in THF (50 mL) was degassed and purged with H₂ for 3 times, and then the mixture was stirred at 25° C. for 1 hr under H₂ atmosphere (15 psi). LC-MS showed desired compound was detected. The mixture was filtered, and the filtrate was concentrated to get the crude product 7-amino-2-methyl-3,4-dihydropyrrolo[1,2-a]pyrazin-1-one (1g, crude) as a yellow solid.

A mixture of 7-amino-2-methyl-3,4-dihydropyrrolo[1,2-a]pyrazin-1-one (400 mg, 2.4 mmol, 1 eq) in THF (10 mL) was degassed and purged with N₂ for 3 times, then LAH (2.5 M, 2.0 mL, 2 eq) was added at 0° C. The mixture was stirred at 25° C. for 1 hr under N₂ atmosphere. LC-MS showed desired compound was detected. The mixture was quenched by sat. MgSO₄ (1 mL) and then the mixture was filtered, and the filtrate was concentrated to get the crude product 2-methyl-3,4-dihydro-1H-pyrrolo[1,2-a]pyrazin-7-amine (350 mg, crude) as a red solid used into the next step without further purification.

To a solution of 2-methyl-3,4-dihydro-1H-pyrrolo[1,2-a]pyrazin-7-amine (200 mg, 1.3 mmol, 1 eq) in IPA (10 mL) was added tert-butyl 8-methyl-4-(8-methyl-2-methylsulfonyl-7-oxo-pyrido[2,3-d]pyrimidin-6-yl)-2,3-dihydroquinoxaline-1-carboxylate (321.1 mg, 661.3 μmol, 0.5 eq). The mixture was stirred at 60° C. for 1 hr. LC-MS showed desired compound was detected.

The mixture was concentrated to get a residue. The residue was purified by prep-HPLC (FA condition; column: Phenomenex Luna C18 100×30 mm×5 μm; mobile phase: [H₂O (0·2% FA)-ACN]; gradient: 15%-45% B over 8.0 min) to give compound tert-butyl 8-methyl-4-[8-methyl-2-[(2-methyl-3,4-dihydro-1H-pyrrolo[1,2-a]pyrazin-7-yl)amino]-7-oxo-pyrido[2,3-d]pyrimidin-6-yl]-2,3-dihydroquinoxaline-1-carboxylate (130 mg, 233.54 μmol, 17.66% yield) was obtained as a brown solid.

To a solution of tert-butyl 8-methyl-4-[8-methyl-2-[(2-methyl-3,4-dihydro-1H-pyrrolo[1,2-a]pyrazin-7-yl)amino]-7-oxo-pyrido[2,3-d]pyrimidin-6-yl]-2,3-dihydroquinoxaline-1-carboxylate (130 mg, 233.5 μmol, 1 eq) in EtOAc (1 mL) was added HCl/EtOAc (2 mL) (4M). The mixture was stirred at 25° C. for 1 hr, the mixture was stirred at 25° C. for 12 hr. LC-MS showed desired compound was detected. The mixture was concentrated to get the crude product 8-methyl-2-[(2-methyl-3,4-dihydro-1H-pyrrolo[1,2-a]pyrazin-7-yl)amino]-6-(5-methyl-3,4-dihydro-2H-quinoxalin-1-yl)pyrido[2,3-d]pyrimidin-7-one (100 mg, crude, HCl) as a white solid used into the next step without further purification.

To a solution of 8-methyl-2-[(2-methyl-3,4-dihydro-1H-pyrrolo[1,2-a]pyrazin-7-yl)amino]-6-(5-methyl-3,4-dihydro-2H-quinoxalin-1-yl)pyrido[2,3-d]pyrimidin-7-one (80 mg, 162.3 μmol, 1 eq HCl) in DCM (3 mL) was added TEA (65.7 mg, 649.1 μmol, 90.3 μL, 4 eq) and prop-2-enoyl chloride (11.8 mg, 129.8 μmol, 10.55 μL, 0.8 eq). The mixture was stirred at 25° C. for 1 hr. LC-MS showed desired compound was detected. The mixture was concentrated to get a residue. The residue was purified by prep-HPLC (TFA condition;column: Phenomenex luna C18 100× 40 mm× 3 μm; mobile phase: [H₂O (0.1% TFA)-ACN]; gradient: 10%-40% B over 8.0 min) to give compound 8-methyl-2-[(2-methyl-3,4-dihydro-1H-pyrrolo[1,2-a]pyrazin-7-yl)amino]-6-(5-methyl-4-prop-2-enoyl-2,3-dihydroquinoxalin-1-yl)pyrido[2,3-d]pyrimidin-7-one (20 mg, 39.1 μmol, 24.1% yield) as an orange solid.

### Procedure for Preparation of Compound 081

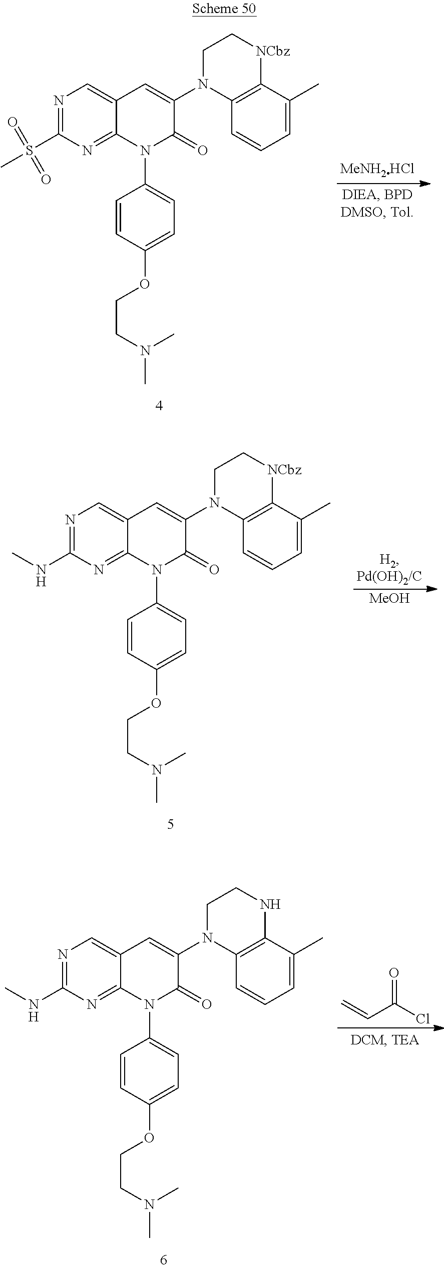

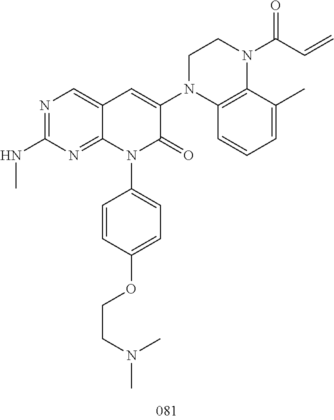 [SMILES: C=CC(=O)N1CCN(c2cc3cnc(NC)nc3n(-c3ccc(OCCN(C)C)cc3)c2=O)c2cccc(C)c21]

To a solution of benzyl 4-[8-[4-[2-(dimethylamino)ethoxy]phenyl]-2-methylsulfonyl-7-oxo-pyrido[2,3-d]pyrimidin-6-yl]-8-methyl-2,3-dihydroquinoxaline-1-carboxylate (170 mg, 254·2 μl mol, 1 eq) in Tol. (2 mL) and DMSO (0.1 mL) was added MeNH₂. HCl (25.7 mg, 381.3 μmol, 1.5 eq) and DIEA (164.3 mg, 1.3 mmol, 221.4 μL, 5 eq). The mixture was stirred at 25° C. for 2 hr. Then BPD (193.7 mg, 762.6 μmol, 3 eq) was added to the mixture. The mixture was stirred at 25° C. for 1 hr. LC-MS showed the desired compound was detected. The reaction mixture was concentrated under reduced pressure to remove solvent to get the residue was purified by prep-HPLC (TFA condition; column: Phenomenex Luna C18 75×30 mm×3 μm; mobile phase: [H₂O (0.1% TFA)-ACN]; gradient: 25%-55% B over 8.0 min) to give benzyl 4-[8-[4-[2-(dimethylamino)ethoxy]phenyl]-2-(methylamino)-7-oxo-pyrido[2,3-d]pyrimidin-6-yl]-8-methyl-2,3-dihydroquinoxaline-1-carboxylate (170 mg, 274.3 μmol, 107.9% yield) as a yellow solid.

To a solution of Pd(OH)₂/C (150 mg, 20% purity) in MeOH (5 mL) was added benzyl 4-[8-[4-[2-(dimethylamino)ethoxy]phenyl]-2-(methylamino)-7-oxo-pyrido[2,3-d]pyrimidin-6-yl]-8-methyl-2,3-dihydroquinoxaline-1-carboxylate (110 mg, 177.5 μmol, 1 eq) under N₂ atmosphere. The suspension was degassed and purged with H₂ for 3 times. The mixture was stirred under H₂ (15 Psi) at 25° C. for 15 min. LC-MS showed the desired compound was detected. The reaction mixture filtered and concentrated under reduced pressure to give a residue to give 8-[4-[2-(dimethylamino)ethoxy]phenyl]-2-(methylamino)-6-(5-methyl-3,4-dihydro-2H-quinoxalin-1-yl)pyrido[2,3-d]pyrimidin-7-one (70 mg, crude) as a yellow solid.

To a solution of 8-[4-[2-(dimethylamino)ethoxy]phenyl]-2-(methylamino)-6-(5-methyl-3,4-dihydro-2H-quinoxalin-1-yl)pyrido[2,3-d]pyrimidin-7-one (70 mg, 144·2 μmol, 1 eq) and TEA (43.8 mg, 432.5 μmol, 60·2 μL, 3 eq) in DCM (1 mL) was added prop-2-enoyl chloride (13.1 mg, 144·2 μmol, 11.7 L, 1 eq) at 0° C. The mixture was stirred at 0° C. for 1 hr. LC-MS showed the desired compound was detected. The reaction mixture was concentrated under reduced pressure to remove solvent to get the residue. The residue was purified by prep-HPLC (TFA condition; column: Phenomenex luna C18 100×40 mm× 3 μm; mobile phase: [H₂O (0.1% TFA)-ACN]; gradient: 10%-40% B over 8.0 min) to give 8-[4-[2-(dimethylamino)ethoxy]phenyl]-2-(methylamino)-6-(5-methyl-4-prop-2-enoyl-2,3-dihydroquinoxalin-1-yl)pyrido[2,3-d]pyrimidin-7-one (13.7 mg, 24.1 μmol, 16.7% yield, 95% purity) as a yellow solid.

### Procedure for Preparation of Compound 084

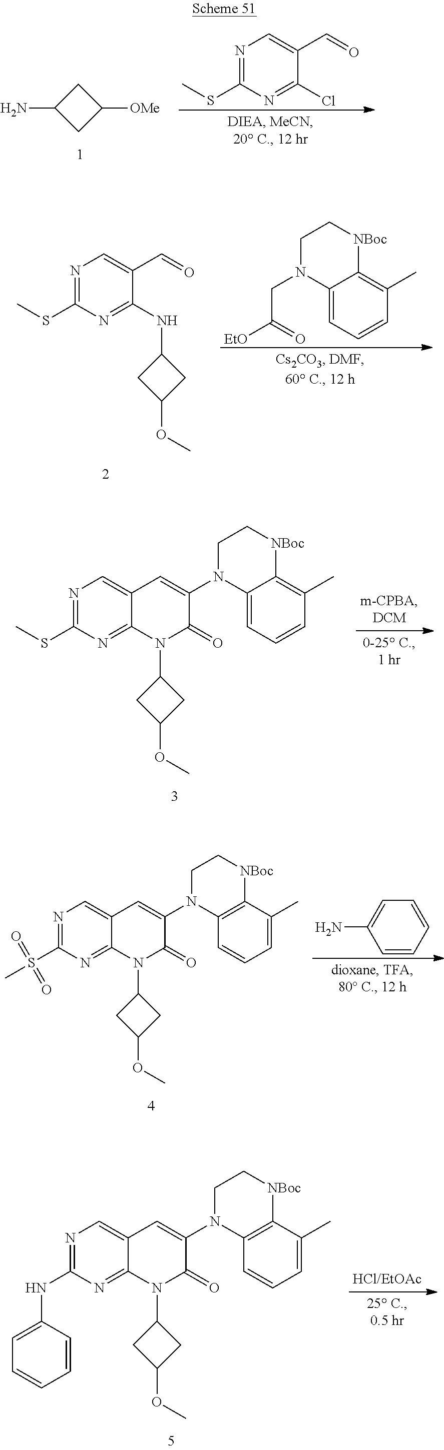

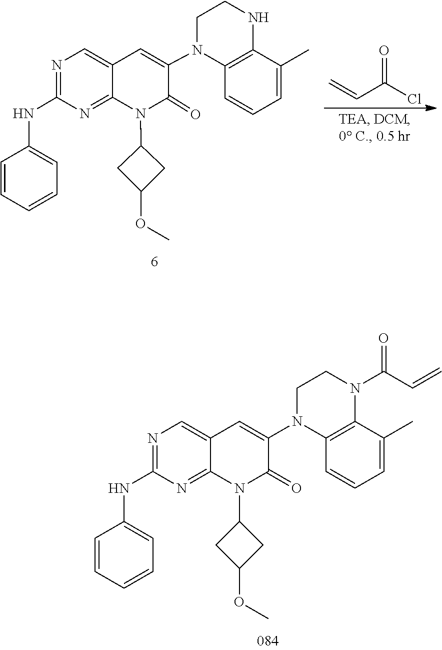

To a solution of 4-chloro-2-methylsulfanyl-pyrimidine-5-carbaldehyde (1.1 g, 6.3 mmol, 1.1 eq) in MeCN (25 mL) was added DIEA (3.7 g, 28.7 mmol, 5.00 mL, 5 eq) and 3-methoxycyclobutanamine;hydrochloride (1.0 g, 5.7 mmol, 1 eq, HCl). The mixture was stirred at 20° C. for 12 hr. LC-MS showed desired compound was detected. The reaction mixture was concentrated under reduced pressure to remove solvent. The residue was purified by flash silica gel chromatography (20 g Silica Flash Column, Eluent of 0˜15% Ethyl acetate/Petroleum ether gradient @ 100 mL/min) to give 4-[(3-methoxycyclobutyl)amino]-2-methylsulfanyl-10 pyrimidine-5-carbaldehyde (734.6 mg, 2.8 mmol, 49.9% yield, 99% purity) as a light yellow oil.

To a solution of 4-[(3-methoxycyclobutyl)amino]-2-methylsulfanyl-pyrimidine-5-carbaldehyde (734.6 mg, 2.0 mmol, 1 eq, TFA) in DMF (20 mL) was added Cs₂CO₃ (1.9 g, 6.0 mmol, 3 eq) and tert-butyl 4-(2-ethoxy-2-oxo-ethyl)-8-methyl-2,3-dihydroquinoxaline-1-carboxylate (668.7 mg, 2.0 mmol, 1 eq). The mixture was stirred at 60° C. for 12 hr. LC-MS showed desired compound was detected. The residue was diluted with H₂O (50 MI) and extracted with ethyl acetate (50 mL×3). The combined organic layers were washed with brine (40 mL× 3), dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 100 mL/min) to give tert-butyl 4-[8-(3-methoxycyclobutyl)-2-methylsulfanyl-7-oxo-pyrido[2,3-d]pyrimidin-6-yl]-8-methyl-2,3-dihydroquinoxaline-1-carboxylate (849.3 mg, 1.1 mmol, 57.5% yield, 71% purity) as a white solid.

To a solution of tert-butyl 4-[8-(3-methoxycyclobutyl)-2-methylsulfanyl-7-oxo-pyrido[2,3-d]pyrimidin-6-yl]-8-methyl-2,3-dihydroquinoxaline-1-carboxylate (849.3 mg, 1.6 mmol, 1 eq) in DCM (20 mL) was added m-CPBA (592.7 mg, 2.9 mmol, 85% purity, 1.8 eq) at 0° C. . . . The resulting mixture was stirred at 25° C. for 1 hr. LC-MS showed desired compound was detected. The reaction mixture was quenched by addition Na₂SO₃ (20 mL), extracted with dichloromethane (20 mL×3). The combined organic layers were washed with NaHCO₃ (20 mL× 2), and dried over Na₂SO₄, filtered and concentrated under reduced pressure to give tert-butyl 4-[8-(3-methoxycyclobutyl)-2-methylsulfinyl-7-oxo-pyrido[2,3-d]pyrimidin-6-yl]-8-methyl-2,3-dihydroquinoxaline-1-carboxylate (914 mg, crude) as a red solid.

To a solution of tert-butyl 4-[8-(3-methoxycyclobutyl)-2-methylsulfinyl-7-oxo-pyrido[2,3-d]pyrimidin-6-yl]-8-methyl-2,3-dihydroquinoxaline-1-carboxylate (220 mg, 407.6 μmol, 1 eq) in dioxane (2 mL) was added TFA (69.7 mg, 611.5 μmol, 45.4 μL, 1.5 eq) and aniline (56.9 mg, 611.5 μmol, 55.7 μL, 1.5 eq). The mixture was stirred at 80° C. for 12 hr. LC-MS showed desired compound was detected. The reaction mixture was concentrated under reduced pressure to remove solvent. The residue was purified by flash silica gel chromatography (12 g Silica Flash Column, Eluent of 0˜50% Ethyl acetate/Petroleum ether gradient @ 100 mL/min) to give tert-butyl 4-[2-anilino-8-(3-methoxycyclobutyl)-7-oxo-pyrido[2,3-d]pyrimidin-6-yl]-8-methyl-2,3-dihydroquinoxaline-1-carboxylate (275 mg, 430.3 μmol, 52.7% yield, 89% purity) as yellow oil.

A solution of tert-butyl 4-[2-anilino-8-(3-methoxycyclobutyl)-7-oxo-pyrido[2,3-d]pyrimidin-6-yl]-8-methyl-2,3-dihydroquinoxaline-1-carboxylate (250 mg, 439.6 μmol, 1 eq) in HCl/EtOAc (6 mL) (4 M) was stirred at 25° C. for 0.5 hr. LC-MS showed desired compound was detected. The mixture was concentrated to get 2-anilino-8-(3-methoxycyclobutyl)-6-(5-methyl-3,4-dihydro-2H-quinoxalin-1-yl)pyrido[2,3-d]pyrimidin-7-one (237 mg, crude, HCl) as a yellow solid.

To a solution of 2-anilino-8-(3-methoxycyclobutyl)-6-(5-methyl-3,4-dihydro-2H-quinoxalin-1-yl)pyrido[2,3-d]pyrimidin-7-one (100 mg, 198.0 μmol, 1 eq, HCl) in DCM (2 mL) was added TEA (60.1 mg, 594.0 μmol, 82.6 μL, 3 eq). The mixture was added dropwise prop-2-enoyl chloride (14.3 mg, 158.4 μmol, 12.8 μL, 0.8 eq) in DCM (1 mL) at 0° C. and stirred at 0° C. for 0.5 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 30%-80% B over 8.0 min) to give 2-anilino-8-(3-methoxycyclobutyl)-6-(5-methyl-4-prop-2-enoyl-2,3-dihydroquinoxalin-1-yl)pyrido[2,3-d]pyrimidin-7-one (50.5 mg, 95.7 μmol, 48.3% yield, 99% purity) as a yellow solid.

### Procedure for Preparation of Compound 087 and Compound 088

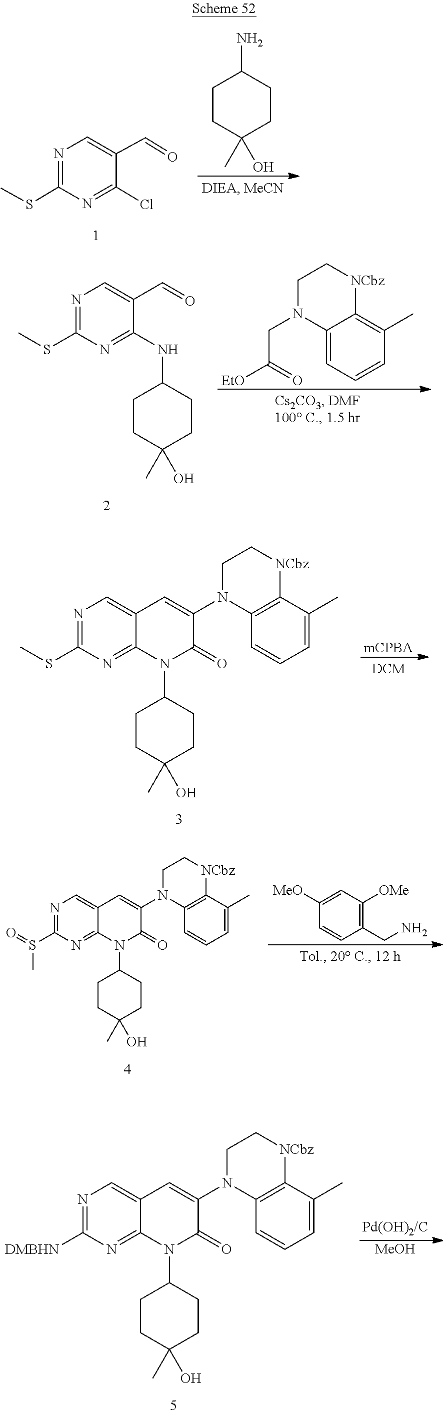

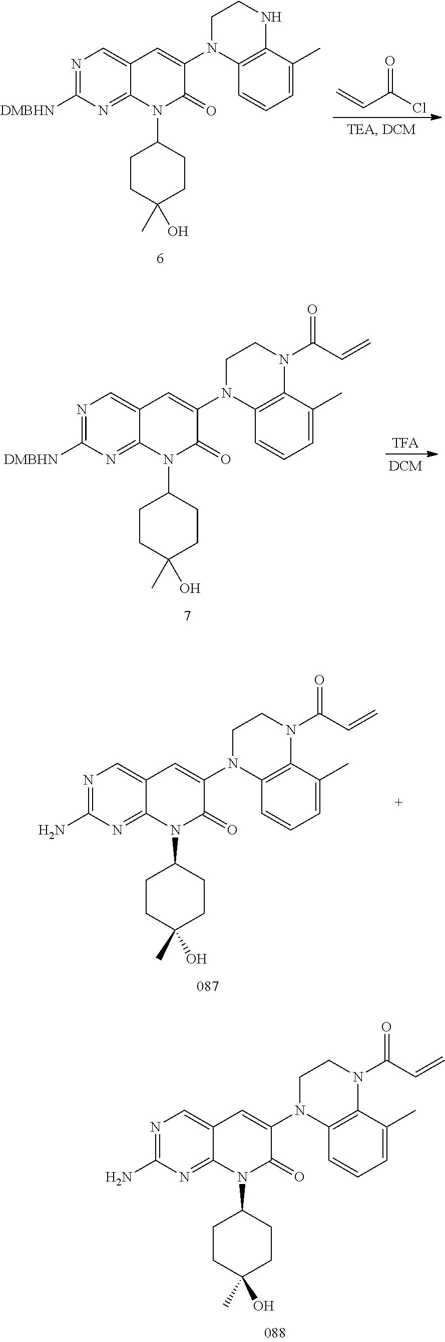

To a solution of 4-chloro-2-methylsulfanyl-pyrimidine-5-carbaldehyde (1.5 g, 7.7 mmol, 1 eq) in MeCN (20 mL) was added DIEA (3.0 g, 23·2 mmol, 4.0 mL, 3 eq) and 4-amino-1-methyl-cyclohexanol (1 g, 7.7 mmol, 1 eq). The mixture was stirred at 20° C. for 16 hr. LC-MS showed desired compound was detected. The reaction mixture was concentrated under reduced pressure to remove solvent. The residue was purified by flash silica gel chromatography (20 g Silica Flash Column, Eluent of 0˜24% Ethyl acetate/Petroleum ether gradient @ 100 mL/min) to give 4-[(4-hydroxy-4-methyl-cyclohexyl)amino]-2-methylsulfanyl-pyrimidine-5-carbaldehyde (1.5 g, 5·2 mmol, 66.6% yield) as yellow oil.

To a solution of 4-[(4-hydroxy-4-methyl-cyclohexyl)amino]-2-methylsulfanyl-pyrimidine-5-carbaldehyde (2.3 g, 8·2 mmol, 1 eq) in DMF (50 mL) was added Cs₂CO₃ (13.3 g, 40.9 mmol, 5 eq) and benzyl 4-(2-ethoxy-2-oxo-ethyl)-8-methyl-2,3-dihydroquinoxaline-1-carboxylate (3.3 g, 9.0 mmol, 1.1 eq). The mixture was stirred at 100° C. for 1.5 hr. LC-MS showed desired compound was detected. The residue was diluted with H₂O (150 mL) and extracted with ethyl acetate (150 mL× 3). The combined organic layers were washed with brine (120 mL× 1), dried over Na₂SO₄, filtered and concentrated under reduced pressure to give a residue. The residue was purified by flash silica gel chromatography (40 g Silica Flash Column, Eluent of 0˜28% Ethyl acetate/Petroleum ether gradient @150 mL/min) to give benzyl 4-[8-(4-hydroxy-4-methyl-cyclohexyl)-2-methylsulfanyl-7-oxo-pyrido[2,3-d]pyrimidin-6-yl]-8-methyl-2,3-dihydroquinoxaline-1-carboxylate (3.4 g, 5.8 mmol, 71.4% yield) as a yellow solid.

To a solution of benzyl 4-[8-(4-hydroxy-4-methyl-cyclohexyl)-2-methylsulfanyl-7-oxo-pyrido[2,3-d]pyrimidin-6-yl]-8-methyl-2,3-dihydroquinoxaline-1-carboxylate (650 mg, 1.1 mmol, 1 eq) in DCM (10 mL) was added mCPBA (225.3 mg, 1.1 mmol, 85% purity, 1 eq) at 0° C. The mixture was stirred at 20° C. for 1 hr. LC-MS showed desired compound was detected. The residue was diluted with Na₂SO₃ (50 mL) and extracted with DCM (50 mL× 3). The combined organic layers were washed with NaHCO₃ (30 mL×1), dried over Na₂SO₄, filtered and concentrated under reduced pressure to give benzyl 4-[8-(4-hydroxy-4-methyl-cyclohexyl)-2-methylsulfinyl-7-oxo-pyrido[2,3-d]pyrimidin-6-yl]-8-methyl-2,3-dihydroquinoxaline-1-carboxylate (600 mg, crude) as a yellow solid.

To a solution of benzyl 4-[8-(4-hydroxy-4-methyl-cyclohexyl)-2-methylsulfinyl-7-oxo-pyrido[2,3-d]pyrimidin-6-yl]-8-methyl-2,3-dihydroquinoxaline-1-carboxylate (200 mg, 332.4 μmol, 1 eq) in Tol. (2 mL) was added (2,4-dimethoxyphenyl) methanamine (66.7 mg, 398.9 μmol, 59.9 μL, 1·2 eq). The mixture was stirred at 20° C. for 12 hr (batch×3). LC-MS showed desired compound was detected. The reaction mixture was concentrated under reduced pressure to remove solvent. The residue was purified by flash silica gel chromatography (12 g Silica Flash Column, Eluent of 0˜42% Ethyl acetate/Petroleum ether gradient @ 100 mL/min) to give benzyl 4-[2-[(2,4-dimethoxyphenyl)methylamino]-8-(4-hydroxy-4-methyl-cyclohexyl)-7-oxo-pyrido[2,3-d]pyrimidin-6-yl]-8-methyl-2,3-dihydroquinoxaline-1-carboxylate (668 mg, 947.8 μmol, 95.0% yield) as a yellow solid.

To a solution of Pd(OH)₂/C (200 mg, 20% purity) in MeOH (2 mL) was added benzyl 4-[2-[(2,4-dimethoxyphenyl)methylamino]-8-(4-hydroxy-4-methyl-cyclohexyl)-7-oxo-pyrido[2,3-d]pyrimidin-6-yl]-8-methyl-2,3-dihydroquinoxaline-1-carboxylate (368 mg, 522.1 μmol, 1 eq) under N₂ atmosphere. The suspension was degassed and purged with H₂ for 3 times. The mixture was stirred under H₂ (15 psi) at 20° C. for 1 hr. LC-MS showed desired compound was detected. The reaction mixture was filtered and concentrated under reduced pressure to give 2-[(2,4-dimethoxyphenyl)methylamino]-8-(4-hydroxy-4-methyl-cyclohexyl)-6-(5-methyl-3,4-dihydro-2H-quinoxalin-1-yl)pyrido[2,3-d]pyrimidin-7-one (250 mg, crude) as a yellow solid. To a solution of 2-[(2,4-dimethoxyphenyl)methylamino]-8-(4-hydroxy-4-methyl-cyclohexyl)-6-(5-methyl-3,4-dihydro-2H-quinoxalin-1-yl)pyrido[2,3-d]pyrimidin-7-one (250 mg, 438.1 μmol, 1 eq) in DCM (3 mL) was added TEA (133.0 mg, 1.3 mmol, 182.9 μL, 3 eq) and prop-2-enoyl chloride (39.6 mg, 438.1 μmol, 35.6 μL, 1 eq) at 0° C. The mixture was stirred at 0° C. for 1 hr. LC-MS showed desired compound was detected. The reaction mixture was concentrated under reduced pressure to remove solvent to give 2-[(2,4-dimethoxyphenyl)methylamino]-8-(4-hydroxy-4-methyl-cyclohexyl)-6-(5-methyl-4-prop-2-enoyl-2,3-dihydroquinoxalin-1-yl)pyrido[2,3-d]pyrimidin-7-one (250 mg, crude) as a yellow solid used into the next step without further purification.

To a solution of 2-[(2,4-dimethoxyphenyl)methylamino]-8-(4-hydroxy-4-methyl-cyclohexyl)-6-(5-methyl-4-prop-2-enoyl-2,3-dihydroquinoxalin-1-yl)pyrido[2,3-d]pyrimidin-7-one (250 mg, 400·2 μmol, 1 eq) in DCM (3 mL) was added TFA (0.5 mL). The mixture was stirred at 20° C. for 12 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 30%-65% B over 8.0 min) and (neutral condition; column: Waters Xbridge BEH C18 100× 30 mm× 10 μm; mobile phase: [H₂O (10 mM NH₄HCO₃)-ACN]; gradient: 25%-60% B over 8.0 min) to give 6-(4-acryloyl-5-methyl-3,4-dihydroquinoxalin-1 (2H)-yl)-2-amino-8-((1r,4r)-4-hydroxy-4-methylcyclohexyl)pyrido[2,3-d]pyrimidin-7 (8H)-one (Compound 087) (12.3 mg, 25.4 μmol, 6.3% yield, 98.0% purity) as a yellow solid and 6-(4-acryloyl-5-methyl-3,4-dihydroquinoxalin-1 (2H)-yl)-2-amino-8-((1s,4s)-4-hydroxy-4-methylcyclohexyl)pyrido[2,3-d]pyrimidin-7 (8H)-one (Compound 088) (21.6 mg, 45.5 μmol, 11.4% yield, 100% purity) as a yellow solid.

### Procedure for Preparation of Compound 086

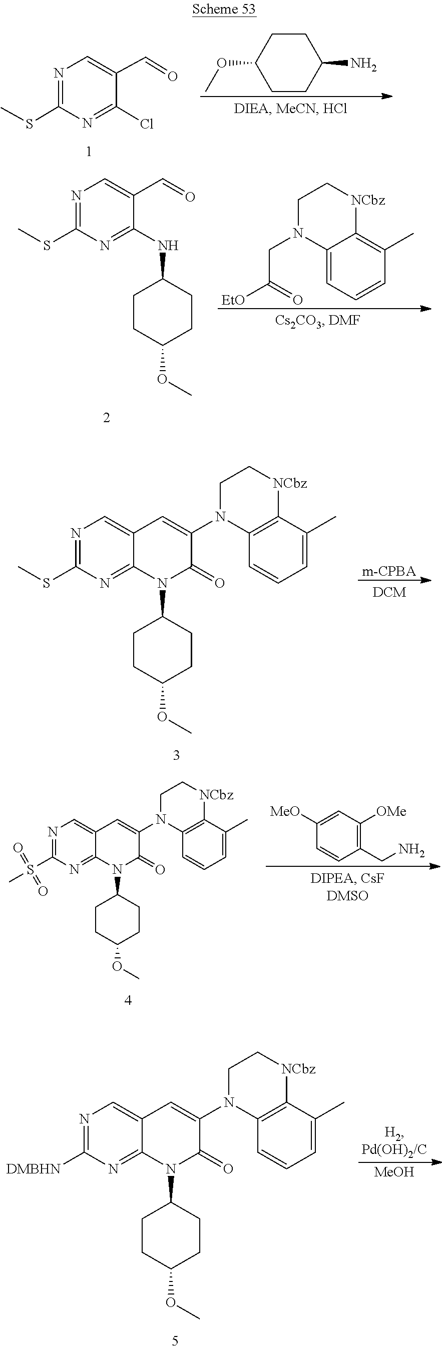

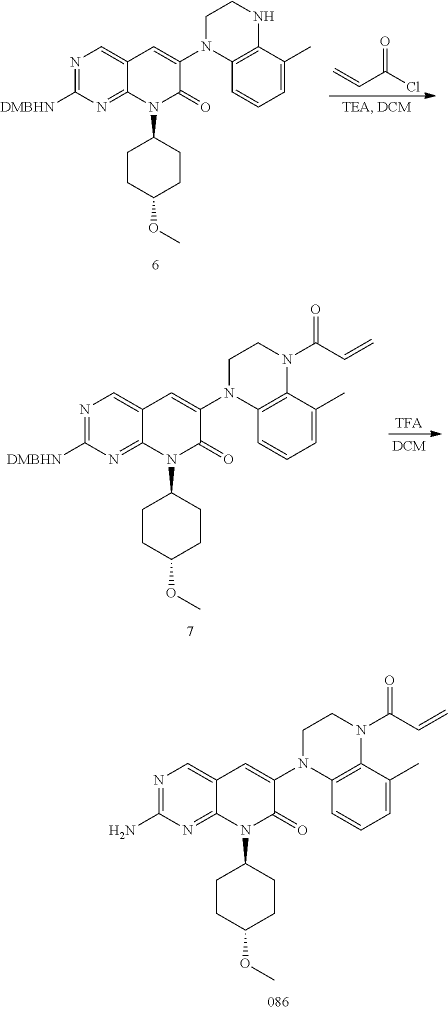

To a solution of 4-chloro-2-methylsulfanyl-pyrimidine-5-carbaldehyde (1.4 g, 7.4 mmol, 1 eq) in MeCN (15 mL) was added DIEA (2.8 g, 22·2 mmol, 3.8 mL, 3 eq) and 4-methoxycyclohexanamine (958.8 mg, 7.4 mmol, 1 eq). The mixture was stirred at 20° C. for 2 hr. Then the mixture was added HCl (10 mL) 1 M and stirred at 20° C. for 0.5 hr. LC-MS showed desired compound was detected. The residue was diluted with NaHCO₃ (10 mL) and extracted with ethyl acetate (10 mL× 3). The combined organic layers were dried over Na₂SO₄, filtered and concentrated under reduced pressure to give a residue. The residue was purified by flash silica gel chromatography (12 g Silica Flash Column, Eluent of 0˜15% Ethyl acetate/Petroleum ether gradient @ 100 mL/min) to give 4-[(4-methoxycyclohexyl)amino]-2-methylsulfanyl-pyrimidine-5-carbaldehyde (727 mg, 2.5 mmol, 34.1% yield, 98% purity) as a white solid.

To a solution of 4-[(4-methoxycyclohexyl)amino]-2-methylsulfanyl-pyrimidine-5-carbaldehyde (727 mg, 2.5 mmol, 1 eq) in DMF (10 mL) was added Cs₂CO₃ (2.5 g, 7.7 mmol, 3 eq) and benzyl 4-(2-ethoxy-2-oxo-ethyl)-8-methyl-2,3-dihydroquinoxaline-1-carboxylate (1.0 g, 2.8 mmol, 1.1 eq). The mixture was stirred at 80° C. for 12 hr. LC-MS showed desired compound was detected. The reaction mixture was extracted with H₂O (30 mL) and ethyl acetate (20 mL× 3). The combined organic layers were washed with brine (20 mL× 3), dried over Na₂SO₄, filtered and concentrated under reduced pressure to give a residue. The residue was purified by flash silica gel chromatography (20 g Silica Flash Column, Eluent of 0˜28% Ethyl acetate/Petroleum ether gradient @ 100 mL/min) to give benzyl 4-[8-(4-methoxycyclohexyl)-2-methylsulfanyl-7-oxo-pyrido[2,3-d]pyrimidin-6-yl]-8-methyl-2,3-dihydroquinoxaline-1-carboxylate (700 mg, 1.1 mmol, 43.9% yield, 95% purity) as a yellow solid.

At 0° C., to a solution of benzyl 4-[8-(4-methoxycyclohexyl)-2-methylsulfanyl-7-oxo-pyrido[2,3-d]pyrimidin-6-yl]-8-methyl-2,3-dihydroquinoxaline-1-carboxylate (700 mg, 1·2 mmol, 1 eq) in DCM (10 mL) was added m-CPBA (291.1 mg, 1.4 mmol, 85% purity, 1·2 eq). The mixture was stirred at 20° C. for 2 hr. LC-MS showed desired compound was detected. The reaction mixture was quenched by addition Na₂SO₃ (20 mL), and then extracted with dichloromethane (10 mL×3). The combined organic layers were washed with NaHCO₃ (10 mL× 3), dried over Na₂SO₄, filtered and concentrated under reduced pressure to give benzyl 4-[8-(4-methoxycyclohexyl)-2-methylsulfonyl-7-oxo-pyrido[2,3-d]pyrimidin-6-yl]-8-methyl-2,3-dihydroquinoxaline-1-carboxylate (780 mg, crude) as an orange solid.

To a solution of benzyl 4-[8-(4-methoxycyclohexyl)-2-methylsulfonyl-7-oxo-pyrido[2,3-d]pyrimidin-6-yl]-8-methyl-2,3-dihydroquinoxaline-1-carboxylate (730 mg, 1.1 mmol, 1 eq) in DMSO (7.3 mL) was added (2,4-dimethoxyphenyl) methanamine (237.1 mg, 1.4 mmol, 213.0 μL, 1·2 eq), DIPEA (458·2 mg, 3.5 mmol, 617.5 μL, 3 eq) and CsF (269·2 mg, 1.7 mmol, 65.4 μL, 1.5 eq). The mixture was stirred at 20° C. for 4 hr. LC-MS showed desired compound was detected. The reaction mixture was extracted with H₂O (10 mL) and ethyl acetate (10 mL× 3). The combined organic layers were washed with brine (10 mL×3), dried over Na₂SO₄, filtered and concentrated under reduced pressure to give a residue. The residue was purified by prep-TLC (SiO₂, Petroleum ether/Ethyl acetate=1/1) to give benzyl 4-[2-[(2,4-dimethoxyphenyl)methylamino]-8-(4-methoxycyclohexyl)-7-oxo-pyrido[2,3-d]pyrimidin-6-yl]-8-methyl-2,3-dihydroquinoxaline-1-carboxylate (550 mg, 756.9 μmol, 64.0% yield, 97% purity) as a yellow solid.

To a solution of Pd(OH)₂/C (250 mg, 20% purity) in MeOH (10 mL) was added benzyl 4-[2-[(2,4-dimethoxyphenyl)methylamino]-8-(4-methoxycyclohexyl)-7-oxo-pyrido[2,3-d]pyrimidin-6-yl]-8-methyl-2,3-dihydroquinoxaline-1-carboxylate (500.0 mg, 709.4 μmol, 1 eq) under N₂ atmosphere. The suspension was degassed and purged with H₂ for 3 times. The mixture was stirred under H₂ (15 Psi.) at 20° C. for 4 hr. LC-MS showed desired compound was detected. The reaction mixture was filtered and concentrated under reduced pressure to give 2-[(2,4-dimethoxyphenyl)methylamino]-8-(4-methoxycyclohexyl)-6-(5-methyl-3,4-dihydro-2H-quinoxalin-1-yl)pyrido[2,3-d]pyrimidin-7-one (170 mg, crude) as a yellow solid.

At 0° C., to a solution of 2-[(2,4-dimethoxyphenyl)methylamino]-8-(4-methoxycyclohexyl)-6-(5-methyl-3,4-dihydro-2H-quinoxalin-1-yl)pyrido[2,3-d]pyrimidin-7-one (170 mg, 297.8 μmol, 1 eq) in DCM (3 mL) was added TEA (90.4 mg, 893.6 μmol, 124.3 μL, 3 eq) and prop-2-enoyl chloride (26.9 mg, 297.8 μmol, 24·2 μL, 1 eq). The mixture was stirred at 0° C. for 1 hr. LC-MS showed desired compound was detected. The reaction mixture was concentrated under N₂ to give 2-[(2,4-dimethoxyphenyl)methylamino]-8-(4-methoxycyclohexyl)-6-(5-methyl-4-prop-2-enoyl-2,3-dihydroquinoxalin-1-yl)pyrido[2,3-d]pyrimidin-7-one (250 mg, crude) as a yellow solid.

To a solution of 2-[(2,4-dimethoxyphenyl)methylamino]-8-(4-methoxycyclohexyl)-6-(5-methyl-4-prop-2-enoyl-2,3-dihydroquinoxalin-1-yl)pyrido[2,3-d]pyrimidin-7-one (250 mg, 400.1 μmol, 1 eq) in DCM (3 mL) was added TFA (1.3 g, 12.1 mmol, 0.9 mL, 30·2 eq). The mixture was stirred at 20° C. for 12 hr. LC-MS showed desired compound was detected. The reaction mixture was concentrated under reduced pressure to remove solvent. The residue was purified by prep-HPLC (TFA condition column: Phenomenex luna C18 100×40 mm×3 μm; mobile phase: [H₂O (0.1% TFA)-ACN]; gradient: 20%-50% B over 8.0 min) to give 2-amino-8-(4-methoxycyclohexyl)-6-(5-methyl-4-prop-2-enoyl-2,3-dihydroquinoxalin-1-yl)pyrido[2,3-d]pyrimidin-7-one (41.0 mg, 68.3 μmol, 17.0% yield, 98% purity, TFA) was obtained as a yellow

### Procedure for Preparation of Compound 089

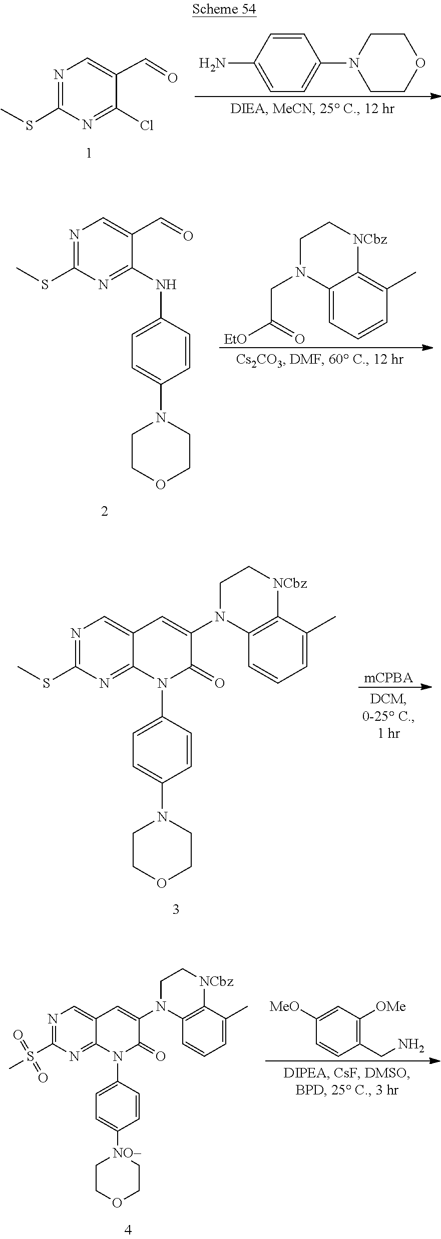

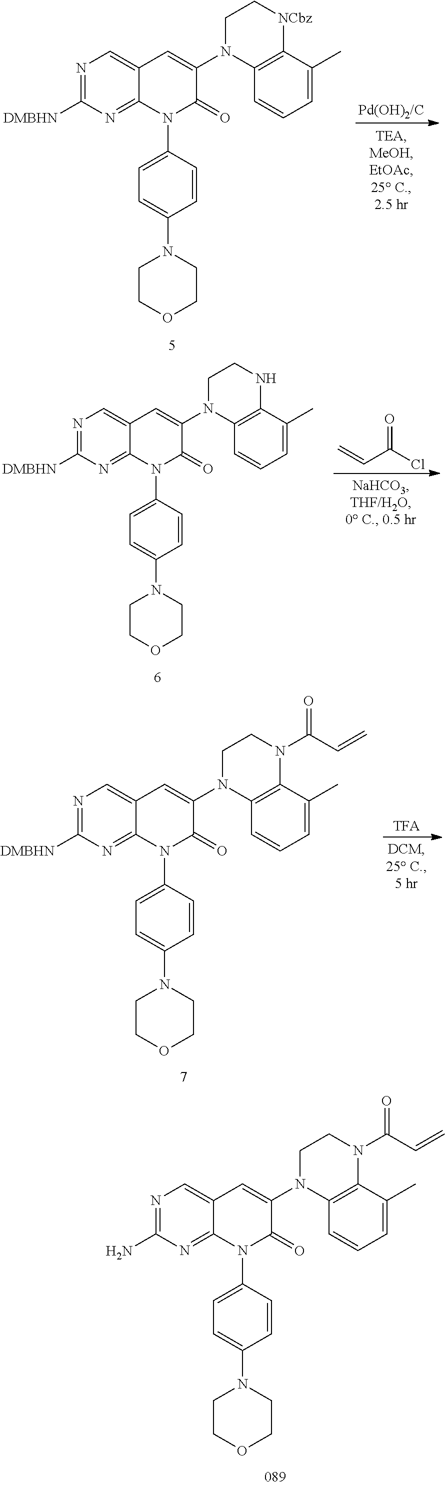

To a solution of 4-chloro-2-methylsulfanyl-pyrimidine-5-carbaldehyde (2.3 g, 12.3 mmol, 1.1 eq) in MeCN (50 mL) was added DIEA (7·2 g, 56.1 mmol, 9.7 mL, 5 eq) and 4-morpholinoaniline (2 g, 11·2 mmol, 1 eq). The mixture was stirred at 25° C. for 12 hr. LC-MS showed desired compound was detected. The reaction mixture was concentrated under reduced pressure to remove solvent. The crude product was triturated with methanol (30 mL) at 25° C. for 1 hr. Compound 2-methylsulfanyl-4-(4-morpholinoanilino)pyrimidine-5-carbaldehyde (3·2 g, 9.4 mmol, 84.5% yield, 98% purity) was obtained as a yellow green solid.

To a solution of 2-methylsulfanyl-4-(4-morpholinoanilino)pyrimidine-5-carbaldehyde (2 g, 6.0 mmol, 1 eq) in DMF (50 mL) was added Cs₂CO₃ (5.9 g, 18.1 mmol, 3 eq) and benzyl 4-(2-ethoxy-2-oxo-ethyl)-8-methyl-2,3-dihydroquinoxaline-1-carboxylate (2·2 g, 6.0 mmol, 1 eq). The mixture was stirred at 60° C. for 12 hr. LC-MS showed desired compound was detected. The residue was diluted with H₂O (20 mL) and extracted with ethyl acetate (10 mL×3). The combined organic layers were washed with brine (10 mL× 3), dried over Na₂SO₄, filtered, and concentrated under reduced pressure to give a residue. The residue was purified by flash silica gel chromatography (12 g Silica Flash Column, Eluent of 0˜28% Ethyl acetate/Petroleum ether gradient @ 100 mL/min) to give benzyl 8-methyl-4-[2-methylsulfanyl-8-(4-morpholinophenyl)-7-oxo-pyrido[2,3-d]pyrimidin-6-yl]-2,3-dihydroquinoxaline-1-carboxylate (2.5 g, 3.5 mmol, 58.5% yield, 90% purity) as a yellow solid.

To a solution of benzyl 8-methyl-4-[2-methylsulfanyl-8-(4-morpholinophenyl)-7-oxo-pyrido[2,3-d]pyrimidin-6-yl]-2,3-dihydroquinoxaline-1-carboxylate (1 g, 1.5 mmol, 1 eq) in DCM (10 mL) was added m-CPBA (479.7 mg, 2.3 mmol, 85% purity, 1.5 eq) at 0° C. The mixture was stirred at 25° C. for 1 hr. LC-MS showed desired compound was detected. The reaction mixture was quenched by addition Na₂SO₃ (20 mL), extracted with dichloromethane (20 mL× 3). The combined organic layers were washed with NaHCO₃ (20 mL× 2), and dried over Na₂SO₄, filtered and concentrated under reduced pressure to give benzyl 8-methyl-4-[2-methylsulfinyl-8-[4-(4-oxidomorpholin-4-ium-4-yl)phenyl]-7-oxo-pyrido[2,3-d]pyrimidin-6-yl]-2,3-dihydroquinoxaline-1-carboxylate (1.0 g, crude) as a yellow solid.

To a solution of benzyl 8-methyl-4-[2-methylsulfinyl-8-[4-(4-morpholinophenyl-4-ium-4-yl)phenyl]-7-oxo-pyrido[2,3-d]pyrimidin-6-yl]-2,3-dihydroquinoxaline-1-carboxylate (1.0 g, 1.5 mmol, 1 eq) in DMSO (10 mL) was added (2,4-dimethoxyphenyl) methanamine (315.9 mg, 1.8 mmol, 283.9 μL, 1·2 eq), DIEA (610.5 mg, 4.7 mmol, 822.8 μL, 3 eq) and CsF (358.8 mg, 2.3 mmol, 87·2 μL, 1.5 eq). The mixture was stirred at 25° C. for 2 hr. The mixture was added BPD (1·2 g, 4.7 mmol, 3 eq) and stirred at 25° C. for 1 hr. LC-MS showed desired compound was detected. The residue was diluted with H₂O 30 mL and extracted with ethyl acetate (20 mL×3). The combined organic layers were washed with brine (20 mL×2), dried over Na₂SO₄, filtered and concentrated under reduced pressure to give a residue. The residue was purified by flash silica gel chromatography (12 g Silica Flash Column, Eluent of 0˜30% Ethyl acetate/Petroleum ether gradient @ 100 mL/min) to give benzyl 4-[2-[(2,4-dimethoxyphenyl)methylamino]-8-(4-morpholinophenyl)-7-oxo-pyrido[2,3-d]pyrimidin-6-yl]-8-methyl-2,3-dihydroquinoxaline-1-carboxylate (580 mg, 723·2 μmol, 45.9% yield, 94% purity) as a yellow solid.

To a solution of Pd(OH)₂ (200 mg, 20% purity) in MeOH (2 mL) was added benzyl 4-[2-[(2,4-dimethoxyphenyl)methylamino]-8-(4-morpholinophenyl)-7-oxo-pyrido[2,3-d]pyrimidin-6-yl]-8-methyl-2,3-dihydroquinoxaline-1-carboxylate (300 mg, 397.9 μmol, 1 eq) in EtOAc (2 mL) and MeOH (2 mL) under N₂ atmosphere. The suspension was added TEA (70 mg, 691.7 μmol, 96·2 μL, 1.7 eq) to adjust pH to 7-8 under N₂ atmosphere. The suspension was degassed and purged with H₂ for 3 times. The mixture was stirred under H₂ (15 Psi) at 25° C. for 2.5 hr. LC-MS showed desired compound was detected. The reaction mixture was filtered and concentrated under reduced pressure to give 2-[(2,4-dimethoxyphenyl)methylamino]-6-(5-methyl-3,4-dihydro-2H-quinoxalin-1-yl)-8-(4-morpholinophenyl)pyrido[2,3-d]pyrimidin-7-one (246 mg, crude) as a red solid.

To a solution of 2-[(2,4-dimethoxyphenyl)methylamino]-6-(5-methyl-3,4-dihydro-2H-quinoxalin-1-yl)-8-(4-morpholinophenyl)pyrido[2,3-d]pyrimidin-7-one (100 mg, 152.4 μmol, 1 eq, HCl) in DCM (2 mL) was added TEA (46·2 mg, 457·2 μmol, 63.6 μL, 3 eq). The mixture was added dropwise prop-2-enoyl chloride (13.7 mg, 152.4 μmol, 12.3 μL, 1 eq) in DCM (0.1 mL) at 0° C. and stirred at 0° C. for 0.5 hr. LC-MS showed desired compound was detected. The reaction mixture was concentrated under reduced pressure to give compound 2-[(2,4-dimethoxyphenyl)methylamino]-6-(5-methyl-4-prop-2-enoyl-2,3-dihydroquinoxalin-1-yl)-8-(4-morpholinophenyl)pyrido[2,3-d]pyrimidin-7-one (102 mg, crude) as a yellow solid.

A solution of 2-[(2,4-dimethoxyphenyl)methylamino]-6-(5-methyl-4-prop-2-enoyl-2,3-dihydroquinoxalin-1-yl)-8-(4-morpholinophenyl)pyrido[2,3-d]pyrimidin-7-one (102 mg, 151.3 μmol, 1 eq) in TFA (0.6 mL) and DCM (2 mL) was stirred at 25° C. for 5 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min) to give 2-amino-6-(5-methyl-4-prop-2-enoyl-2,3-dihydroquinoxalin-1-yl)-8-(4-morpholinophenyl)pyrido[2,3-d]pyrimidin-7-one (34.3 mg, 65.0 μmol, 42.9% yield, 99% purity) as a yellow solid.

### Procedure for Preparation of Compound 104

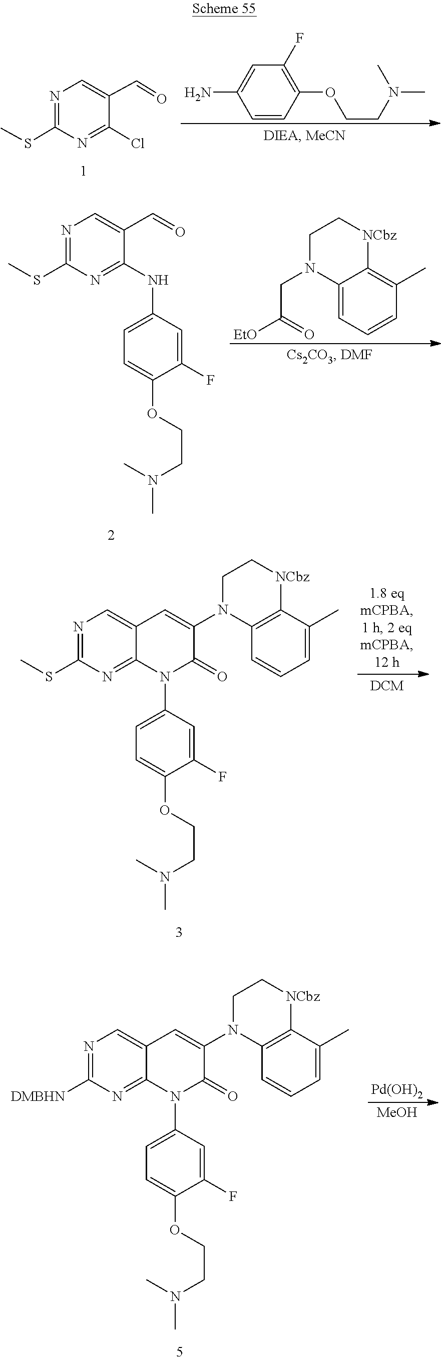

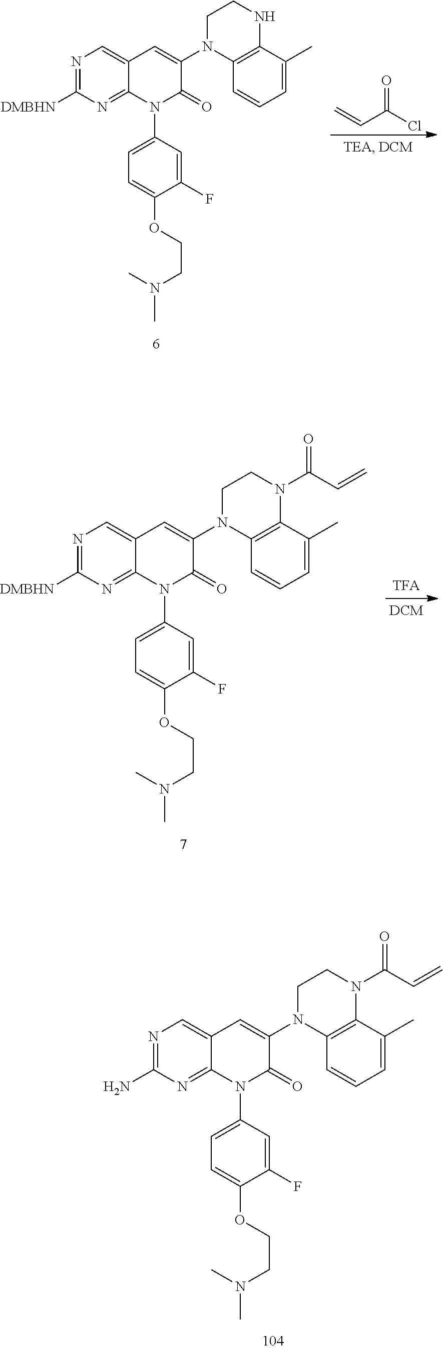

To a solution of 4-chloro-2-methylsulfanyl-pyrimidine-5-carbaldehyde (1.8 g, 9.7 mmol, 1 eq) in MeCN (30 mL) was added DIEA (3.8 g, 29·2 mmol, 5.1 mL, 3 eq) and 4-[2-(dimethylamino)ethoxy]-3-fluoro-aniline (1.9 g, 9.7 mmol, 1 eq). The mixture was stirred at 20° C. for 2 hr. LC-MS showed desired compound was detected. The reaction mixture was concentrated under reduced pressure to remove solvent. The residue was purified by flash silica gel chromatography (20 g Silica Flash Column, Eluent of 0˜100% Ethyl acetate/Petroleum ether gradient @ 100 mL/min, Eluent of 0˜10% Methanol/Dichloromethane @ 100 mL/min) to give 4-[4-[2-(dimethylamino)ethoxy]-3-fluoro-anilino]-2-methylsulfanyl-pyrimidine-5-carbaldehyde (3.5 g, 8.3 mmol, 85.4% yield, 84% purity) as a brown solid.

To a solution of 4-[4-[2-(dimethylamino)ethoxy]-3-fluoro-anilino]-2-methylsulfanyl-pyrimidine-5-carbaldehyde (3.5 g, 9.9 mmol, 1·2 eq) in DMF (50 mL) was added Cs₂CO₃ (8.1 g, 24.8 mmol, 3 eq) and benzyl 4-(2-ethoxy-2-oxo-ethyl)-8-methyl-2,3-dihydroquinoxaline-1-carboxylate (3.0 g, 8.3 mmol, 1 eq). The mixture was stirred at 60° C. for 12 hr. LC-MS showed desired compound was detected. The residue was diluted with H₂O (300 mL) and extracted with ethyl acetate (300 mL× 4). The combined organic layers were washed with brine (300 mL× 3), dried over Na₂SO₄, filtered, and concentrated under reduced pressure to give a residue. The residue was purified by flash silica gel chromatography (40 g Silica Flash Column, Eluent of 0˜100% Ethyl acetate/Petroleum ether gradient, Eluent of 0˜10% Methanol/Dichloromethane @ 120 mL/min) to give benzyl 4-[8-[4-[2-(dimethylamino)ethoxy]-3-fluoro-phenyl]-2-methylsulfanyl-7-oxo-pyrido[2,3-d]pyrimidin-6-yl]-8-methyl-2,3-dihydroquinoxaline-1-carboxylate (3.4 g, 5.1 mmol, 62.0% yield) as a yellow solid.

To a solution of benzyl 4-[8-[4-[2-(dimethylamino)ethoxy]-3-fluoro-phenyl]-2-methylsulfanyl-7-oxo-pyrido[2,3-d]pyrimidin-6-yl]-8-methyl-2,3-dihydroquinoxaline-1-carboxylate (500 mg, 763.7 μmol, 1 eq) in DCM (10 mL) was added mCPBA (279.1 mg, 1.4 mmol, 85% purity, 1.8 eq) at 0° C. The mixture was stirred at 20° C. for 1 hr. Then the mixture was added mCPBA (310.0 mg, 1.5 mmol, 85% purity, 2 eq) at 0° C. The mixture was stirred at 20° C. for 12 hr. LC-MS showed desired compound was detected. The residue was diluted with Na₂SO₃ (20 mL) and extracted with Dichloromethane (30 mL× 3). The combined organic layers were washed with NaHCO₃ (20 mL×3), dried over Na₂SO₄, filtered and concentrated under reduced pressure to give a residue to give 2-[4-[6-(4-benzyloxycarbonyl-5-methyl-2,3-dihydroquinoxalin-1-yl)-2-methylsulfonyl-7-oxo-pyrido[2,3-d]pyrimidin-8-yl]-2-fluoro-phenoxy]—N,N-dimethyl-ethanamine oxide (460 mg, crude) as a yellow solid.

To a solution of 2-[4-[6-(4-benzyloxycarbonyl-5-methyl-2,3-dihydroquinoxalin-1-yl)-2-methylsulfonyl-7-oxo-pyrido[2,3-d]pyrimidin-8-yl]-2-fluoro-phenoxy]—N, N-dimethyl-ethanamine oxide (460 mg, 654.6 μmol, 1 eq) in DMSO (6 mL) was added (2,4-dimethoxyphenyl) methanamine (131.3 mg, 785.5 μmol, 118.0 μL, 1·2 eq), DIPEA (253.8 mg, 2.0 mmol, 342.0 μL, 3 eq) and CsF (99.4 mg, 654.6 μmol, 24·2 μL, 1 eq). The mixture was stirred at 20° C. for 1 hr. Then the mixture was added BPD (498.7 mg, 2.0 mmol, 3 eq), the mixture was stirred at 20° C. for 1 hr. LC-MS showed desired compound was detected. The residue was diluted with H₂O (100 mL) and extracted with ethyl acetate (100 mL× 4). The combined organic layers were washed with brine (80 mL× 3), dried over Na₂SO₄, filtered, and concentrated under reduced pressure to give a residue. The residue was purified by flash silica gel chromatography (20 g Silica Flash Column, Eluent of 0˜8% Methanol/Dichloromethane ether gradient @ 100 mL/min) to give benzyl 4-[2-[(2,4-dimethoxyphenyl)methylamino]-8-[4-[2-(dimethylamino)ethoxy]-3-fluoro-phenyl]-7-oxo-pyrido[2,3-d]pyrimidin-6-yl]-8-methyl-2,3-dihydroquinoxaline-1-carboxylate (2·23 g) as a yellow oil.

To a solution of Pd(OH)₂ (600 mg, 20% purity) in MeOH (30 mL) was added benzyl 4-[2-[(2,4-dimethoxyphenyl)methylamino]-8-[4-[2-(dimethylamino)ethoxy]-3-fluoro-phenyl]-7-oxo-pyrido[2,3-d]pyrimidin-6-yl]-8-methyl-2,3-dihydroquinoxaline-1-carboxylate (1 g, 1.3 mmol, 1 eq) and TEA (261.5 mg, 2.6 mmol, 359.7 μL, 2 eq) under N₂ atmosphere. The suspension was degassed and purged with H₂ for 3 times. The mixture was stirred under H₂ (15 Psi) at 25° C. for 1 hr. LC-MS showed desired compound was detected. The reaction mixture filtered and concentrated under reduced pressure to give 2-[(2,4-dimethoxyphenyl)methylamino]-8-[4-[2-(dimethylamino)ethoxy]-3-fluoro-phenyl]-6-(5-methyl-3,4-dihydro-2H-quinoxalin-1-yl)pyrido[2,3-d]pyrimidin-7-one (750 mg, crude) as a yellow oil.

To a solution of 2-[(2,4-dimethoxyphenyl)methylamino]-8-[4-[2-(dimethylamino)ethoxy]-3-fluoro-phenyl]-6-(5-methyl-3,4-dihydro-2H-quinoxalin-1-yl)pyrido[2,3-d]pyrimidin-7-one (300 mg, 469.0 μmol, 1 eq) in DCM (5 mL) was added TEA (142.4 mg, 1.4 mmol, 195.8 μL, 3 eq) and prop-2-enoyl chloride (42.4 mg, 469.0 μmol, 38.1 μL, 1 eq) at 0° C. The mixture was stirred at 0° C. for 0.5 hr. LC-MS showed desired compound was detected. The reaction mixture was concentrated under reduced pressure to remove solvent. The residue was purified by prep-HPLC (TFA condition. column: Phenomenex luna C18 100×40 mm×3 μm; mobile phase: [H₂O (0.1% TFA)-ACN]; gradient: 15%-45% B over 8.0 min) to give 2-[(2,4-dimethoxyphenyl)methylamino]-8-[4-[2-(dimethylamino)ethoxy]-3-fluoro-phenyl]-6-(5-methyl-4-prop-2-enoyl-2,3-dihydroquinoxalin-1-yl)pyrido[2,3-d]pyrimidin-7-one (68 mg, 98.0 μmol, 20.9% yield) as a yellow solid.

A mixture of 2-[(2,4-dimethoxyphenyl)methylamino]-8-[4-[2-(dimethylamino)ethoxy]-3-fluoro-phenyl]-6-(5-methyl-4-prop-2-enoyl-2,3-dihydroquinoxalin-1-yl)pyrido[2,3-d]pyrimidin-7-one (58 mg, 83.6 μmol, 1 eq) in DCM (2.5 mL) and TFA (0.5 mL) was stirred at 25° C. for 12 hr. LC-MS showed desired compound was detected. The reaction mixture was concentrated under reduced pressure to remove solvent. The residue was purified by prep-HPLC (TFA condition. column: Phenomenex luna C18 100×40 mm×3 μm; mobile phase: [H₂O (0.1% TFA)-ACN]; gradient: 10%-40% B over 8.0 min) to give 2-amino-8-[4-[2-(dimethylamino)ethoxy]-3-fluoro-phenyl]-6-(5-methyl-4-prop-2-enoyl-2,3-dihydroquinoxalin-1-yl)pyrido[2,3-d]pyrimidin-7-one (22.58 mg, 34.34 μmol, 41.07% yield, 100% purity, TFA) as a yellow solid.

### Procedure for Preparation of Compound 092

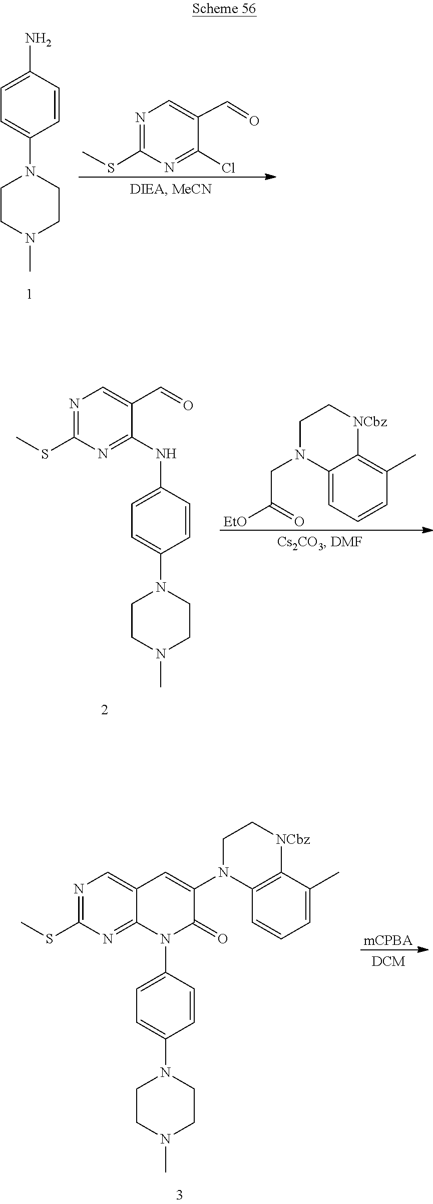

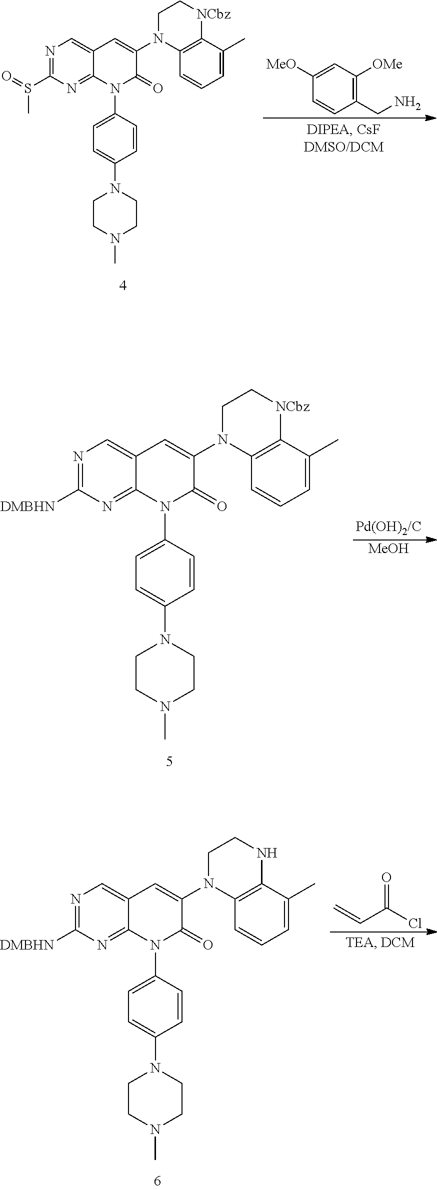

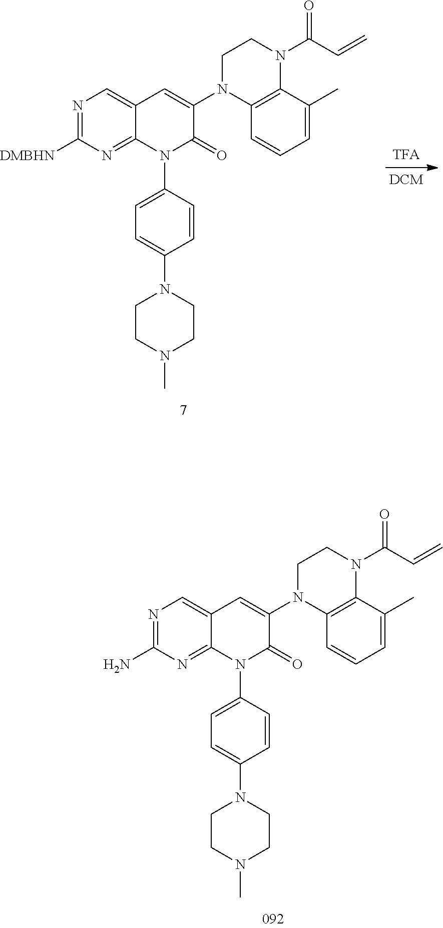

To a solution of 4-chloro-2-methylsulfanyl-pyrimidine-5-carbaldehyde (2.0 g, 10.5 mmol, 1 eq) in MeCN (40 mL) was added DIEA (4.1 g, 31.4 mmol, 5.5 mL, 3 eq) and 4-(4-methylpiperazin-1-yl)aniline (2 g, 10.5 mmol, 1 eq). The mixture was stirred at 20° C. for 4 hr. LC-MS showed desired compound was detected. The reaction mixture was concentrated under reduced pressure to give 4-[4-(4-methylpiperazin-1-yl)anilino]-2-methylsulfanyl-pyrimidine-5-carbaldehyde (4.1 g, crude) as a yellowish-green solid.

To a solution of 4-[4-(4-methylpiperazin-1-yl)anilino]-2-methylsulfanyl-pyrimidine-5-carbaldehyde (1.1 g, 3.3 mmol, 1 eq) in DMF (40 mL) was added Cs₂CO₃ (3·2 g, 9.8 mmol, 3 eq) and benzyl 4-(2-ethoxy-2-oxo-ethyl)-8-methyl-2,3-dihydroquinoxaline-1-carboxylate (1·2 g, 3.3 mmol, 1 eq). The mixture was stirred at 60° C. for 2 hr. LC-MS showed desired compound was detected. The residue was diluted with H₂O (50 mL) and extracted with ethyl acetate (50 mL× 3). The combined organic layers were washed with brine (20 mL× 2), dried over Na₂SO₄, filtered, and concentrated under reduced pressure to give a residue. The residue was purified by flash silica gel chromatography (20 g Silica Flash Column, Eluent of 0˜100% Ethyl acetate/Petroleum ether gradient @150 mL/min) to give benzyl 8-methyl-4-[8-[4-(4-methylpiperazin-1-yl)phenyl]-2-methylsulfanyl-7-oxo-pyrido[2,3-d]pyrimidin-6-yl]-2,3-dihydroquinoxaline-1-carboxylate (1.7 g, 2.6 mmol, 78·2% yield) as a yellow solid.

To a solution of benzyl 8-methyl-4-[8-[4-(4-methylpiperazin-1-yl)phenyl]-2-methylsulfanyl-7-oxo-pyrido[2,3-d]pyrimidin-6-yl]-2,3-dihydroquinoxaline-1-carboxylate (930 mg, 1.4 mmol, 1 eq) in DCM (20 mL) was added mCPBA (1.1 g, 5.5 mmol, 85% purity, 3.8 eq) at 0° C. The mixture was stirred at 20° C. for 12 hr. LC-MS showed desired compound was detected. The residue was diluted with Na₂SO₃ (150 mL) and extracted with DCM (150 mL× 3). The combined organic layers were washed with NaHCO₃ (100 mL× 1), dried over Na₂SO₄, filtered, and concentrated under reduced pressure to give benzyl 8-methyl-4-[8-[4-(4-methylpiperazin-1-yl)phenyl]-2-methylsulfinyl-7-oxo-pyrido[2,3-d]pyrimidin-6-yl]-2,3-dihydroquinoxaline-1-carboxylate (437 mg, crude) as a yellow solid.

To a solution of benzyl 8-methyl-4-[8-[4-(4-methylpiperazin-1-yl)phenyl]-2-methylsulfinyl-7-oxo-pyrido[2,3-d]pyrimidin-6-yl]-2,3-dihydroquinoxaline-1-carboxylate (240 mg, 353.1 μmol, 1 eq) in DMSO (1 mL) and DCM (1 mL) was added (2,4-dimethoxyphenyl) methanamine (70.8 mg, 423.7 μmol, 63.7 μL, 1·2 eq), DIPEA (136.9 mg, 1.1 mmol, 184.5 L, 3 eq) and CsF (53.6 mg, 353.1 μmol, 13.0 μL, 1 eq). The mixture was stirred at 20° C. for 1 hr (batch×2). LC-MS showed desired compound was detected. The reaction mixture was concentrated under column: Phenomenex luna C18 100×40 mm×3 μm; mobile phase: [H₂O (0.1% TFA)-ACN]; gradient: 35%-65% B over 8.0 min) to give benzyl 4-[2-[(2,4-dimethoxyphenyl)methylamino]-8-[4-(4-methylpiperazin-1-yl)phenyl]-7-oxo-pyrido[2,3-d]pyrimidin-6-yl]-8-methyl-2,3-dihydroquinoxaline-1-carboxylate (100 mg, 130.4 μmol, 12.3% yield) as a yellow solid.

To a solution of Pd(OH)₂/C (40 mg, 20% purity) in MeOH (5 mL) was added benzyl 4-[2-[(2,4-dimethoxyphenyl)methylamino]-8-[4-(4-methylpiperazin-1-yl)phenyl]-7-oxo-pyrido[2,3-d]pyrimidin-6-yl]-8-methyl-2,3-dihydroquinoxaline-1-carboxylate (80 mg, 104.3 μmol, 1 eq) under N2 atmosphere. The suspension was degassed and purged with H₂ for 3 times. The mixture was stirred under H₂ (15 Psi) at 20° C. for 1 hr. LC-MS showed desired compound was detected. The reaction mixture was filtered and concentrated under reduced pressure to give 2-[(2,4-dimethoxyphenyl)methylamino]-6-(5-methyl-3,4-dihydro-2H-quinoxalin-1-yl)-8-[4-(4-methylpiperazin-1-yl)phenyl]pyrido[2,3-d]pyrimidin-7-one (80 mg, crude) as a yellow solid.

To a solution of 2-[(2,4-dimethoxyphenyl)methylamino]-6-(5-methyl-3,4-dihydro-2H-quinoxalin-1-yl)-8-[4-(4-methylpiperazin-1-yl)phenyl]pyrido[2,3-d]pyrimidin-7-one (40 mg, 63·2 μmol, 1 eq) in DCM (1 mL) was added TEA (19·2 mg, 189.7 μmol, 26.4 μL, 3 eq) and prop-2-enoyl chloride (5.7 mg, 63·2 μmol, 5.1 μL, 1 eq) at 0° C. The mixture was stirred at 0° C. for 1 hr. LC-MS showed desired compound was detected. The reaction mixture was concentrated under N2 to remove solvent to give 2-[(2,4-dimethoxyphenyl)methylamino]-8-[4-(4-methylpiperazin-1-yl)phenyl]-6-(5-methyl-4-prop-2-enoyl-2,3-dihydroquinoxalin-1-yl)pyrido[2,3-d]pyrimidin-7-one (40 mg, crude) as yellow solid used into the next step without further purification.

To a solution of 2-[(2,4-dimethoxyphenyl)methylamino]-8-[4-(4-methylpiperazin-1-yl)phenyl]-6-(5-methyl-4-prop-2-enoyl-2,3-dihydroquinoxalin-1-yl)pyrido[2,3-d]pyrimidin-7-one (70 mg, 101.9 μmol, 1 eq) in DCM (1 mL) was added TFA (460.5 mg, 4.0 mmol, 0.3 mL, 39.6 eq). The mixture was stirred at 20° C. for 12 hr. LC-MS showed desired compound was detected. The reaction mixture was concentrated under reduced pressure to remove solvent. The residue was purified by prep-HPLC (neutral condition; column: Waters Xbridge BEH C18 100×30 mm× 10 μm; mobile phase: [H₂O (10 mM NH₄HCO₃)-ACN]; gradient: 25%-55% B over 8.0 min) to give 2-amino-8-[4-(4-methylpiperazin-1-yl)phenyl]-6-(5-methyl-4-prop-2-enoyl-2,3-dihydroquinoxalin-1-yl)pyrido[2,3-d]pyrimidin-7-one (3.5 mg, 6.4 μmol, 6.3% yield, 100% purity) as a yellow solid.

### Procedure for Preparation of Compound 101

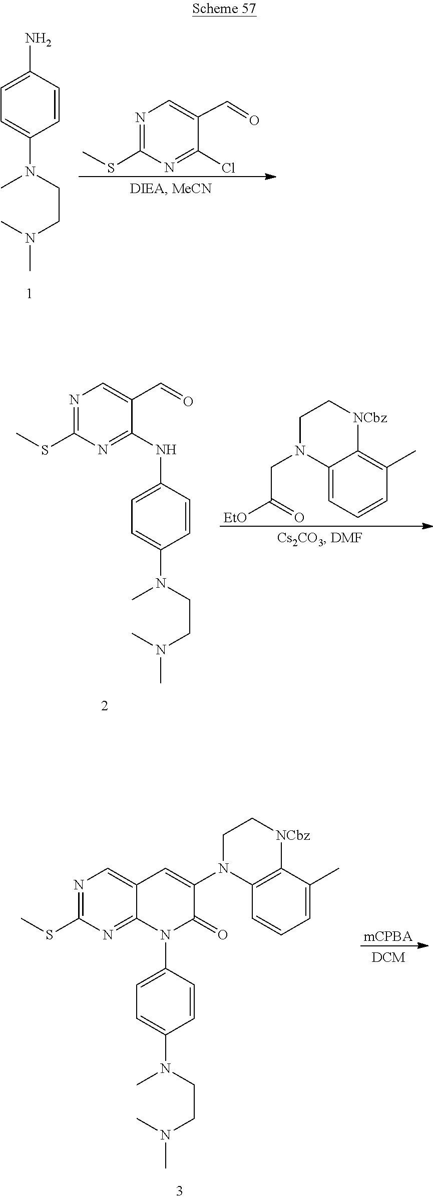

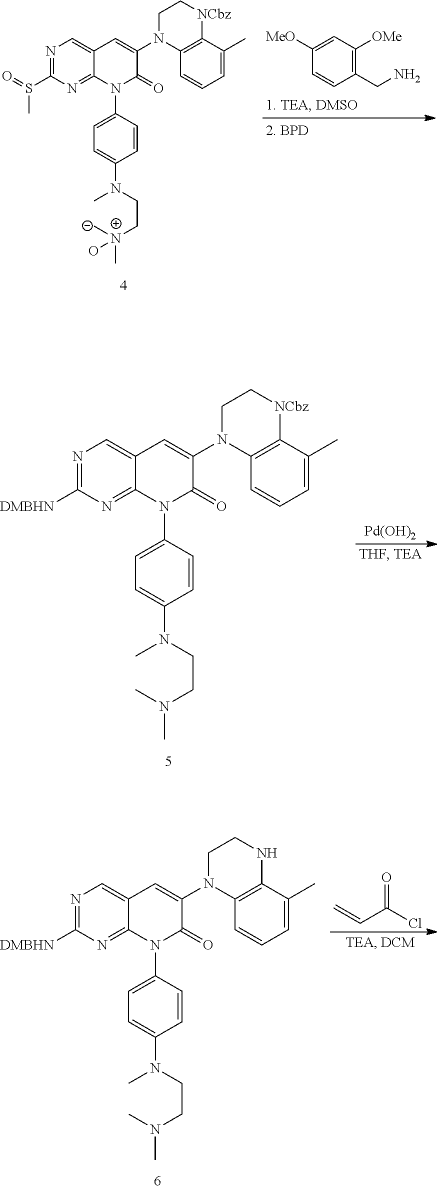

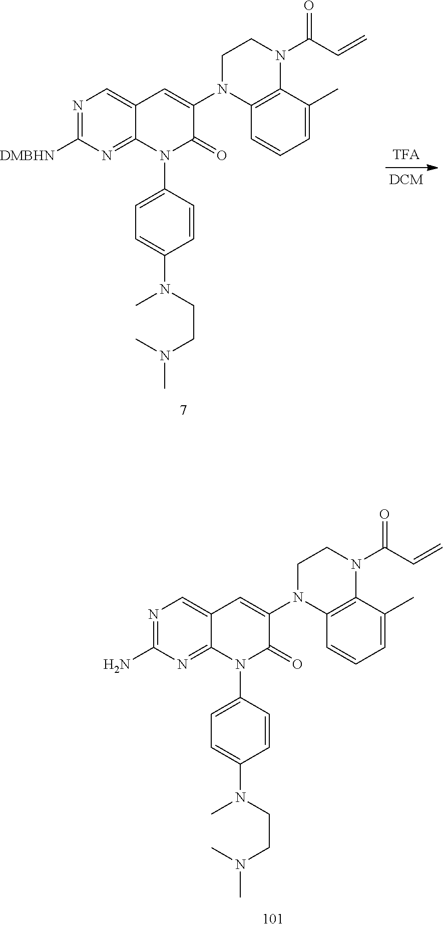

To a solution of 4-chloro-2-methylsulfanyl-pyrimidine-5-carbaldehyde (894.6 mg, 4.7 mmol, 1 eq) in MeCN (20 mL) was added DIEA (1.8 g, 14·2 mmol, 2.48 mL, 3 eq) and N1-[2-(dimethylamino)ethyl]—N1-methyl-benzene-1,4-diamine (1.1 g, 5.7 mmol, 1·2 eq). The mixture was stirred at 25° C. for 12 hr. LC-MS showed desired compound was detected. The mixture was concentrated to get a residue and the residue was purified by reversed-phase HPLC (330 g Agela C18, Eluent of MeOH/10% TFA gradient @70 mL/min) to give compound 4-[4-[2-(dimethylamino)ethyl-methyl-amino]anilino]-2-methylsulfanyl-pyrimidine-5-carbaldehyde (1 g, 2.9 mmol, 61.0% yield) as a black solid.

To a solution of 4-[4-[2-(dimethylamino)ethyl-methyl-amino]anilino]-2-methylsulfanyl-pyrimidine-5-carbaldehyde (1 g, 2.9 mmol, 1 eq) in DMF (15 mL) was added Cs₂CO₃ (2.8 g, 8.7 mmol, 3 eq) and benzyl 4-(2-ethoxy-2-oxo-ethyl)-8-methyl-2,3-dihydroquinoxaline-1-carboxylate (1.1 g, 2.9 mmol, 1 eq). The mixture was stirred at 60° C. for 12 hr. LC-MS showed desired compound was detected. The reaction mixture was partitioned between brine (20 mL) and ethyl acetate (10 mL× 3). The organic phase was separated, washed with brine (10 mL× 2), dried over Na₂SO₄, filtered, and concentrated under reduced pressure to give a residue. The residue was purified by column chromatography (SiO₂, Petroleum ether/Ethyl acetate=3/1 to 0/1 to EtOAc/MeOH=10/1) to give compound benzyl 4-[8-[4-[2-(dimethylamino)ethyl-methyl-amino]phenyl]-2-methylsulfanyl-7-oxo-pyrido[2,3-d]pyrimidin-6-yl]-8-methyl-2,3-dihydroquinoxaline-1-carboxylate (0.9 g, 1.4 mmol, 47.9% yield) as an orange solid.

To a solution of benzyl 4-[8-[4-[2-(dimethylamino)ethyl-methyl-amino]phenyl]-2-methylsulfanyl-7-oxo-pyrido[2,3-d]pyrimidin-6-yl]-8-methyl-2,3-dihydroquinoxaline-1-carboxylate (800 mg, 1·2 mmol, 1 eq) in DCM (10 mL) was added mCPBA (499.9 mg, 2.5 mmol, 85% purity, 2 eq). The mixture was stirred at 25° C. for 1 hr. LC-MS showed desired compound was detected. The mixture was quenched by sat. Na₂SO₃ (5 mL), then the mixture was extracted by dichloromethane (10 mL×3), the combined organic layers was washed by sat. NaHCO₃ (10 mL× 3), then dried by Na₂SO₄, filtered and the filtrate was concentrated to get a residue. The crude product 2-[4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-methyl-anilino]—N, N-dimethyl-ethanamine oxide (900 mg, crude) as a red solid used into the next step without further purification.

To a solution of 2-[4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-methyl-anilino]—N, N-dimethyl-ethanamine oxide (700 mg, 1.03 mmol, 1 eq) in DMSO (10 mL) was added TEA (311.7 mg, 3.1 mmol, 428.7 μL, 3 eq) and (2,4-dimethoxyphenyl) methan amine (206.0 mg, 1·2 mmol, 185.1 μL, 1·2 eq). The mixture was stirred at 25° C. for 1 hr. Then BPD (782·2 mg, 3.1 mmol, 3 eq) was added, the mixture was stirred at 25° C. for 2 hr. LC-MS showed desired compound was detected. Water (20 mL) was added to the mixture and then extracted by ethyl acetate (10 mL× 3), the combined organic layers was washed by brine (10 mL× 2), then dried by Na₂SO₄, filtered and the filtrate was concentrated to get a residue. The residue was purified by prep-HPLC (TFA condition; column: Phenomene×luna C18 100×40 mm×3 μm; mobile phase: [H₂O (0.1% TFA)-ACN];gradient: 35%-65% B over 8.0 min) to give compound benzyl 4-[2-[(2,4-dimethoxyphenyl)methylamino]-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (320 mg, 416·2 μmol, 40.5% yield) as an orange solid.

To a mixture of Pd(OH)₂ (10 mg, 20% purity), TEA (15.8 mg, 156.1 μmol, 21.7 μL, 2 eq) in THF (2 mL) was added benzyl 4-[2-[(2,4-dimethoxyphenyl)methylamino]-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (60 mg, 78.0 μmol, 1 eq), the mixture was degassed and purged with H₂ for 3 times, and then the mixture was stirred at 20° C. for 2 hr under H₂ (15 Psi) atmosphere. LC-MS showed desired compound was detected. The mixture was filtered, and the filtrate was concentrated to get the crude product 2-[(2,4-dimethoxyphenyl)methylamino]-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (50 mg, crude) as an orange solid.

To a solution of 2-[(2,4-dimethoxyphenyl)methylamino]-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (50 mg, 78.8 μmol, 1 eq) in DCM (2 mL) was added TEA (23.9 mg, 236.3 μmol, 32.9 μL, 3 eq) and prop-2-enoyl chloride (7.1 mg, 78.8 μmol, 6.4 μL, 1 eq). The mixture was stirred at 25° C. for 1 hr. Then prop-2-enoyl chloride (3.6 mg, 39.4 μmol, 3·2 μL, 0.5 eq) was added, the mixture was stirred at 25° C. for 1 hr. LC-MS showed desired compound was detected. The mixture was concentrated to get the crude product 2-[(2,4-dimethoxyphenyl)methylamino]-8-[4-[2-(dimethylamino)ethyl-methyl-amino]phenyl]-6-(5-methyl-4-prop-2-enoyl-2,3-dihydroquinoxalin-1-yl)pyrido[2,3-d]pyrimidin-7-one (50 mg, crude) as a red solid used into the next step without further purification.

To a solution of 2-[(2,4-dimethoxyphenyl)methylamino]-8-[4-[2-(dimethylamino)ethyl-methyl-amino]phenyl]-6-(5-methyl-4-prop-2-enoyl-2,3-dihydroquinoxalin-1-yl)pyrido[2,3-d]pyrimidin-7-one (50 mg, 72.6 μmol, 1 eq) in DCM (1 mL) was added TFA (0.3 mL). The mixture was stirred at 25° C. for 12 hr. LC-MS showed desired compound was detected. The mixture was concentrated to get a residue. The residue was purified by prep-HPLC (TFA condition; column: Phenomenex luna C18 100×40 mm×3 μm; mobile phase: [H₂O (0.1% TFA)-ACN]; gradient: 15%-45% B over 8.0 min) to give compound 2-amino-8-[4-[2-(dimethylamino)ethyl-methyl-amino]phenyl]-6-(5-methyl-4-prop-2-enoyl-2,3-dihydroquinoxalin-1-yl)pyrido[2,3-d]pyrimidin-7-one (5 mg, 9·2 μmol, 12.7% yield, 99.7% purity) as a brown solid

### Procedure for Preparation of Compound 093

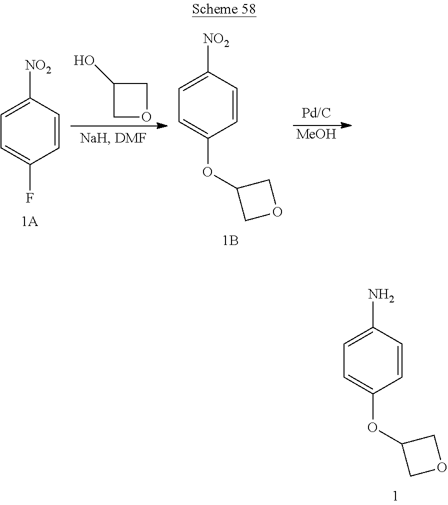

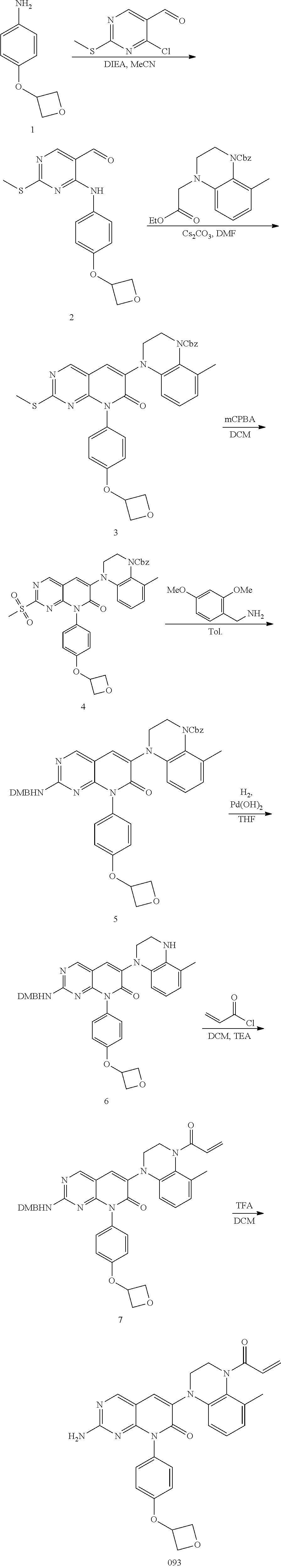

To a solution of oxetan-3-ol (1.1 g, 14.1 mmol, 1 eq) in DMF (30 mL) was added NaH (680.3 mg, 17.0 mmol, 60% purity, 1·2 eq) at 0° C. The mixture was stirred at 0° C. for 30 min under N₂. Then 1-fluoro-4-nitro-benzene (2 g, 14.1 mmol, 1.5 mL, 1 eq) was added to the mixture, the mixture was stirred at 0° C. for 1 hr under N₂. LC-MS showed the desired compound was detected. The reaction mixture was partitioned between NH₄CI (100 mL) and ethyl acetate (100 mL× 3). The organic phase was separated, washed with brine (50 mL× 3), dried over Na₂SO₄, filtered and concentrated under reduced pressure to give a residue. The residue was purified by flash silica gel chromatography (20 g Silica Flash Column, Eluent of 0˜20% Ethyl acetate/Petroleum ether gradient @ 100 mL/min) to give compound 3-(4-nitrophenoxy) oxetane (2·2 g, 11·2 mmol, 79.5% yield) as a yellow solid.

To a solution of Pd/C (0.9 g, 10% purity) in MeOH (20 mL) was added 3-(4-nitrophenoxy) oxetane (1.8 g, 9·2 mmol, 1 eq) under N₂ atmosphere. The suspension was degassed and purged with H₂ for 3 times. The mixture was stirred under H₂ (15 Psi) at 25° C. for 12 hr. LC-MS showed the desired compound was detected. The reaction mixture was filtered, and the filtrate concentrated in vacuum to give compound 4-(oxetan-3-yloxy) aniline (1.6 g, crude) as a black solid.

To a solution of 4-(oxetan-3-yloxy) aniline (1.5 g, 9.0 mmol, 1 eq) in MeCN (30 mL) was added DIEA (3.5 g, 27.0 mmol, 4.7 mL, 3 eq) and 4-chloro-2-methylsulfanyl-pyrimidine-5-carbaldehyde (1.7 g, 9.0 mmol, 1 eq). The mixture was stirred at 25° C. for 12 hr. LC-MS showed the desired compound was detected. The reaction mixture was concentrated under reduced pressure to give a residue. The crude product was triturated with methyl tert-butyl ether (50 mL) at 25° C. for 30 min. The reaction mixture was filtered and the filter cake dried in vacuum to give compound 2-methylsulfanyl-4-[4-(oxetan-3-yloxy) anilino]pyrimidine-5-carbaldehyde (2.8 g, 8.8 mmol, 97.9% yield) as brown solid.

To a solution of 2-methylsulfanyl-4-[4-(oxetan-3-yloxy) anilino]pyrimidine-5-carbaldehyde (2.8 g, 8.8 mmol, 1 eq) in DMF (60 mL) was added Cs₂CO₃ (8.6 g, 26.4 mmol, 3 eq) and benzyl 4-(2-ethoxy-2-oxo-ethyl)-8-methyl-2,3-dihydroquinoxaline-1-carboxylate (3·2 g, 8.8 mmol, 1 eq). The mixture was stirred at 60° C. for 12 hr. LC-MS showed the desired compound was detected.

The reaction mixture was partitioned between H₂O (100 mL) and ethyl acetate (100 mL× 3). The organic phase was separated, washed with brine (50 mL× 3), dried over Na₂SO₄, filtered, and concentrated under reduced pressure to give a residue. The residue was purified by flash silica gel chromatography (40 g Silica Flash Column, Eluent of 0˜40% Ethyl acetate/Petroleum ether gradient @ 120 mL/min) to give compound benzyl 8-methyl-4-[2-methylsulfanyl-8-[4-(oxetan-3-yloxy)phenyl]-7-oxo-pyrido[2,3-d]pyrimidin-6-yl]-2,3-dihydroquinoxaline-1-carboxylate (3 g, 4.8 mmol, 54.7% yield) as a yellow solid.

To a solution of benzyl 8-methyl-4-[2-methylsulfanyl-8-[4-(oxetan-3-yloxy)phenyl]-7-oxo-pyrido[2,3-d]pyrimidin-6-yl]-2,3-dihydroquinoxaline-1-carboxylate (3 g, 4.8 mmol, 1 eq) in DCM (50 mL) was added m-CPBA (1.5 g, 7.3 mmol, 85% purity, 1.5 eq) at 0° C. The mixture was stirred at 0° C. for 2 hr. LC-MS showed the desired compound was detected. The reaction mixture was partitioned between Na₂SO₃ (50 mL) and dichloromethane (50×3 mL). The organic phase was separated, washed with NaHCO₃ (50 mL× 3), dried over Na₂SO₄, filtered, and concentrated under reduced pressure to give compound benzyl 8-methyl-4-[2-methylsulfonyl-8-[4-(oxetan-3-yloxy)phenyl]-7-oxo-pyrido[2,3-d]pyrimidin-6-yl]-2,3-dihydroquinoxaline-1-carboxylate (3 g, crude) as a yellow solid.

A mixture of benzyl 8-methyl-4-[2-methylsulfonyl-8-[4-(oxetan-3-yloxy)phenyl]-7-oxo-pyrido[2,3-d]pyrimidin-6-yl]-2,3-dihydroquinoxaline-1-carboxylate (500 mg, 764.8 μmol, 1 eq), (2,4-dimethoxyphenyl) methanamine (153.4 mg, 917.8 μmol, 137.8 μL, 1·2 eq) in Tol. (6 mL) was stirred at 25° C. for 12 hr. LC-MS showed the desired compound was detected. The reaction mixture was concentrated under reduced pressure to give a residue. The residue was purified by flash silica gel chromatography (20 g Silica Flash Column, Eluent of 0˜60% Ethyl acetate/Petroleum ether gradient @ 120 mL/min) to give compound benzyl 4-[2-[(2,4-dimethoxyphenyl)methylamino]-8-[4-(oxetan-3-yloxy)phenyl]-7-oxo-pyrido[2,3-d]pyrimidin-6-yl]-8-methyl-2,3-dihydroquinoxaline-1-carboxylate (1 g) as a yellow solid.

To a solution of Pd(OH)₂ (100 mg, 20% purity) in THF (5 mL) was added benzyl 4-[2-[(2,4-dimethoxyphenyl)methylamino]-8-[4-(oxetan-3-yloxy)phenyl]-7-oxo-pyrido[2,3-d]pyrimidin-6-yl]-8-methyl-2,3-dihydroquinoxaline-1-carboxylate (200 mg, 269.9 μmol, 1 eq) and TEA (54.6 mg, 539.9 μmol, 75.1 μL, 2 eq) under N₂ atmosphere. The suspension was degassed and purged with H₂ for 3 times. The mixture was stirred under H₂ (15 Psi) at 25° C. for 1.5 hr. LC-MS showed the desired compound was detected. The reaction mixture filtered and concentrated under reduced pressure to give compound 2-[(2,4-dimethoxyphenyl)methylamino]-6-(5-methyl-3,4-dihydro-2H-quinoxalin-1-yl)-8-[4-(oxetan-3-yloxy)phenyl]pyrido[2,3-d]pyrimidin-7-one (170 mg, crude) as a brown solid.

To a solution of 2-[(2,4-dimethoxyphenyl)methylamino]-6-(5-methyl-3,4-dihydro-2H-quinoxalin-1-yl)-8-[4-(oxetan-3-yloxy)phenyl]pyrido[2,3-d]pyrimidin-7-one (170 mg, 280·2 μmol, 1 eq) in DCM (3 mL) was added TEA (56.7 mg, 560.4 μmol, 78.0 μL, 2 eq) and prop-2-enoyl chloride (27 mg, 298.3 μmol, 24·2 μL, 1 eq) at 0° C. The mixture was stirred at 0° C. for 1 hr. LC-MS showed the desired compound was detected. The reaction mixture was concentrated under reduced pressure to give compound 2-[(2,4-dimethoxyphenyl)methylamino]-6-(5-methyl-4-prop-2-enoyl-2,3-dihydroquinoxalin-1-yl)-8-[4-(oxetan-3-yloxy)phenyl]pyrido[2,3-d]pyrimidin-7-one (170 mg, crude) was obtained as a yellow solid.

To a solution of 2-[(2,4-dimethoxyphenyl)methylamino]-6-(5-methyl-4-prop-2-enoyl-2,3-dihydroquinoxalin-1-yl)-8-[4-(oxetan-3-yloxy)phenyl]pyrido[2,3-d]pyrimidin-7-one (170 mg, 257.3 μmol, 1 eq) in DCM (2 mL) was added TFA (0.3 mL). The mixture was stirred at 25° C. for 12 hr. LC-MS showed the desired compound was detected. The reaction mixture was concentrated under reduced pressure to give a residue. The residue was purified by prep-HPLC (TFA condition, column: Phenomenex luna C18 100×40 mm×3 μm; mobile phase: [H₂O (0.1% TFA)-ACN]; gradient: 35%-65% B over 8.0 min) to give compound 2-amino-6-(5-methyl-4-prop-2-enoyl-2,3-dihydroquinoxalin-1-yl)-8-[4-(oxetan-3-yloxy)phenyl]pyrido[2,3-d]pyrimidin-7-one (16.9 mg, 32.8 μmol, 12.7% yield, 99.1% purity) as a white solid.

### Procedure for Preparation of Compound 094

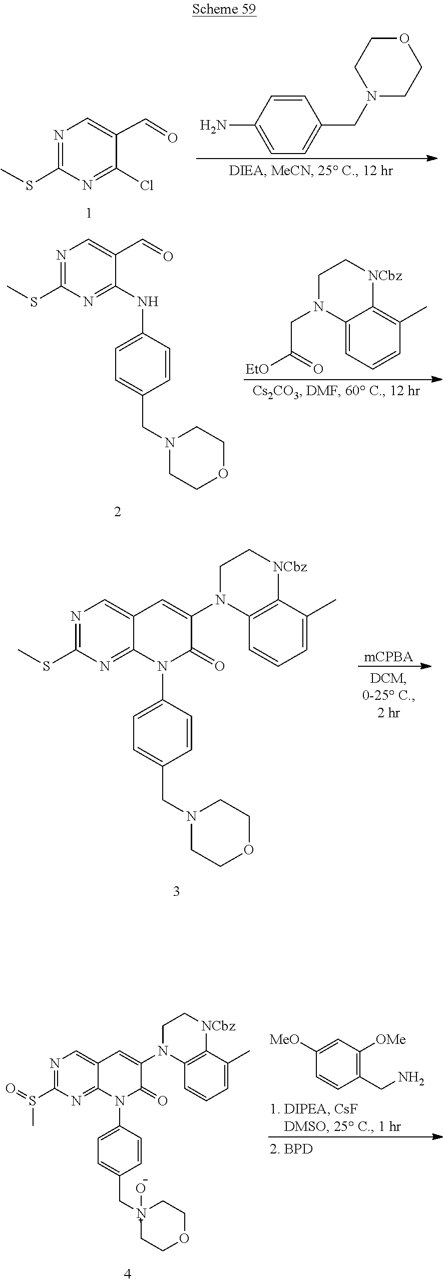

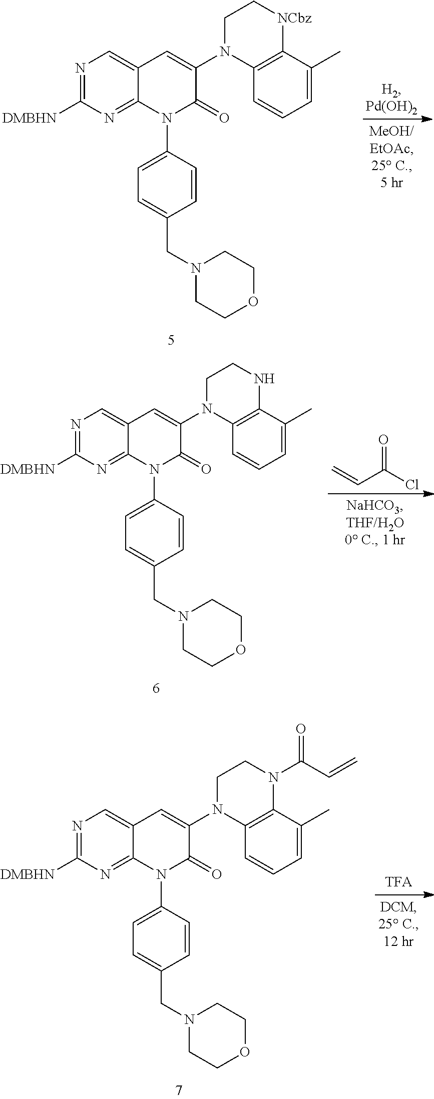

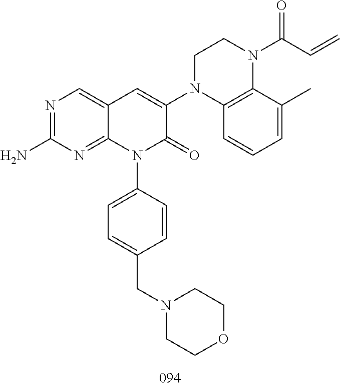 [SMILES: C=CC(=O)N1CCN(c2cc3cnc(N)nc3n(-c3ccc(CN4CCOCC4)cc3)c2=O)c2cccc(C)c21]

To a solution of 4-chloro-2-methylsulfanyl-pyrimidine-5-carbaldehyde (2.1 g, 11.4 mmol, 1.1 eq) in MeCN (50 mL) was added DIEA (4.0 g, 31·2 mmol, 5.4 mL, 3 eq) and 4-(morpholinomethyl)aniline (2 g, 10.4 mmol, 1 eq). The mixture was stirred at 25° C. for 12 hr. LC-MS showed desired compound was detected. The reaction mixture was concentrated under reduced pressure to remove solvent. The crude product was triturated with methanol (30 mL) at 25° C. for 1 hr. Compound 2-methylsulfanyl-4-[4-(morpholinomethyl)anilino]pyrimidine-5-carbaldehyde (2.4 g, 5.7 mmol, 55.1% yield, 81% purity) was obtained as a yellow green solid.

To a solution of 2-methylsulfanyl-4-[4-(morpholinomethyl)anilino]pyrimidine-5-carbaldehyde (2.4 g, 7.0 mmol, 1 eq) in DMF (50 mL) was added Cs₂CO₃ (6.9 g, 21·2 mmol, 3 eq) and benzyl 4-(2-ethoxy-2-oxo-ethyl)-8-methyl-2,3-dihydroquinoxaline-1-carboxylate (2.6 g, 7.0 mmol, 1 eq). The mixture was stirred at 60° C. for 12 hr. LC-MS showed desired compound was detected. The residue was diluted with H₂O 20 mL and extracted with ethyl acetate (10 mL× 3). The combined organic layers were washed with brine (10 mL× 3), dried over Na₂SO₄, filtered, and concentrated under reduced pressure to give a residue. The residue was purified by flash silica gel chromatography (12 g Silica Flash Column, Eluent of 0˜33% Ethyl acetate/Petroleum ether gradient @ 100 mL/min) to give benzyl 8-methyl-4-[2-methylsulfanyl-8-[4-(morpholinomethyl)phenyl]-7-oxo-pyrido[2,3-d]pyrimidin-6-yl]-2,3-dihydroquinoxaline-1-carboxylate (2.8 g, 3.7 mmol, 52.7% yield, 86% purity) as a yellow solid.

To a solution of benzyl 8-methyl-4-[2-methylsulfanyl-8-[4-(morpholinomethyl)phenyl]-7-oxo-pyrido[2,3-d]pyrimidin-6-yl]-2,3-dihydroquinoxaline-1-carboxylate (1.5 g, 2.3 mmol, 1 eq) in DCM (15 mL) was added m-CPBA (704.0 mg, 3.4 mmol, 85% purity, 1.5 eq) at 0° C. The mixture was stirred at 25° C. for 2 hr. LC-MS showed desired compound was detected. The reaction mixture was quenched by addition Na₂SO₃ solution 10 mL, and then diluted with brine (5 mL) and extracted with DCM (20 mL× 3). The combined organic layers were washed with NaHCO₃ solution 45 mL (15 mL×3), dried over Na₂SO₄, filtered, and concentrated under reduced pressure to give benzyl 8-methyl-4-[2-methylsulfonyl-8-[4-(morpholinomethyl)phenyl]-7-oxo-pyrido[2,3-d]pyrimidin-6-yl]-2,3-dihydroquinoxaline-1-carboxylate (1.5 g, crude) as a red solid.

To a solution of benzyl 8-methyl-4-[2-methylsulfonyl-8-[4-(morpholinomethyl)phenyl]-7-oxo-pyrido[2,3-d]pyrimidin-6-yl]-2,3-dihydroquinoxaline-1-carboxylate (850 mg, 1·2 mmol, 1 eq) in DMSO (10 mL) was added (2,4-dimethoxyphenyl) methanamine (250.5 mg, 1.5 mmol, 225.0 μL, 1·2 eq), DIEA (484.1 mg, 3.7 mmol, 652.4 L, 3 eq) and CsF (284.4 mg, 1.8 mmol, 69.1 μL, 1.5 eq). The mixture was stirred at 25° C. for 1 hr. The mixture was added BPD (951·2 mg, 3.7 mmol, 3 eq) and stirred at 25° C. for 1 hr. LC-MS showed desired compound was detected. The residue was diluted with H₂O 10 mL and extracted with ethyl acetate (10 mL× 3). The combined organic layers were washed with brine (10 mL× 2), dried over Na₂SO₄, filtered, and concentrated under reduced pressure to give a residue. The residue was purified by flash silica gel chromatography (12 g Silica Flash Column, Eluent of 0˜100% Ethyl acetate/Petroleum ether gradient @ 100 mL/min) to give benzyl 4-[2-[(2,4-dimethoxyphenyl)methylamino]-8-[4-(morpholinomethyl)phenyl]-7-oxo-pyrido[2,3-d]pyrimidin-6-yl]-8-methyl-2,3-dihydroquinoxaline-1-carboxylate (480 mg, 512.5 μmol, 20.5% yield, 82% purity) as a yellow solid.

To a solution of Pd(OH)₂ (200 mg, 20% purity) in MeOH (2 mL) was added benzyl 4-[2-[(2,4-dimethoxyphenyl)methylamino]-8-[4-(morpholinomethyl)phenyl]-7-oxo-pyrido[2,3-d]pyrimidin-6-yl]-8-methyl-2,3-dihydroquinoxaline-1-carboxylate (300 mg, 390.6 μmol, 1 eq) in

EtOAc (2 mL) and MeOH (2 mL) under N₂ atmosphere. The suspension was added TEA (100 mg, 988·2 μmol, 137.5 μL, 2.5 eq) to adjust pH to 7-8 under N₂ atmosphere. The suspension was degassed and purged with H₂ for 3 times. The mixture was stirred under H₂ (15 Psi) at 25° C. for 5 hr. LC-MS showed desired compound was detected. The reaction mixture was filtered and concentrated under reduced pressure to give 2-[(2,4-dimethoxyphenyl)methylamino]-6-(5-methyl-3,4-dihydro-2H-quinoxalin-1-yl)-8-[4-(morpholinomethyl)phenyl]pyrido[2,3-d]pyrimidin-7-one (238 mg, crude) as a red solid.

To a solution of 2-[(2,4-dimethoxyphenyl)methylamino]-6-(5-methyl-3,4-dihydro-2H-quinoxalin-1-yl)-8-[4-(morpholinomethyl)phenyl]pyrido[2,3-d]pyrimidin-7-one (110 mg, 173.5 μmol, 1 eq) in DCM (2 mL) was added TEA (52.6 mg, 520.7 μmol, 72.4 μL, 3 eq). The mixture was added dropwise prop-2-enoyl chloride (15.7 mg, 173.5 μmol, 14.1 μL, 1 eq) in DCM (0.1 mL) at 0° C. and stirred at 0° C. for 0.5 hr. LC-MS showed 2-[(2,4-dimethoxyphenyl)methylamino]-6-(5-methyl-3,4-dihydro-2H-quinoxalin-1-yl)-8-[4-(morpholinomethyl)phenyl]pyrido[2,3-d]pyrimidin-7-one remained. The mixture was added dropwise prop-2-enoyl chloride (3.1 mg, 3.7 μmol, 2.8 μL, 0·2 eq) in DCM (0.1 mL) at 0° C. and stirred at 0° C. for 0.5 hr. LC-MS showed desired compound was detected. The reaction mixture was concentrated under reduced pressure to remove solvent. Compound 2-[(2,4-dimethoxyphenyl)methylamino]-6-(5-methyl-4-prop-2-enoyl-2,3-dihydroquinoxalin-1-yl)-8-[4-(morpholinomethyl)phenyl]pyrido[2,3-d]pyrimidin-7-one (119 mg, crude) was obtained as a yellow solid.

A solution of 2-[(2,4-dimethoxyphenyl)methylamino]-6-(5-methyl-4-prop-2-enoyl-2,3-dihydroquinoxalin-1-yl)-8-[4-(morpholinomethyl)phenyl]pyrido[2,3-d]pyrimidin-7-one (119 mg, 173.0 μmol, 1 eq) in TFA (0.6 mL) and DCM (2 mL) was stirred at 25° C. for 12 hr. LC-MS showed desired compound was detected. The reaction mixture was concentrated under column: Phenomenex luna C18 100×40 mm×3 μm; mobile phase: [H₂O (0.1% TFA)-ACN]; gradient: 1%-35% B over 8.0 min) to give 2-amino-6-(5-methyl-4-prop-2-enoyl-2,3-dihydroquinoxalin-1-yl)-8-[4-(morpholinomethyl)phenyl]pyrido[2,3-d]pyrimidin-7-one (19.6 mg, 30.1 μmol, 17.4% yield, 100% purity, TFA) as a yellow solid.

### Procedure for Preparation of Compound 103

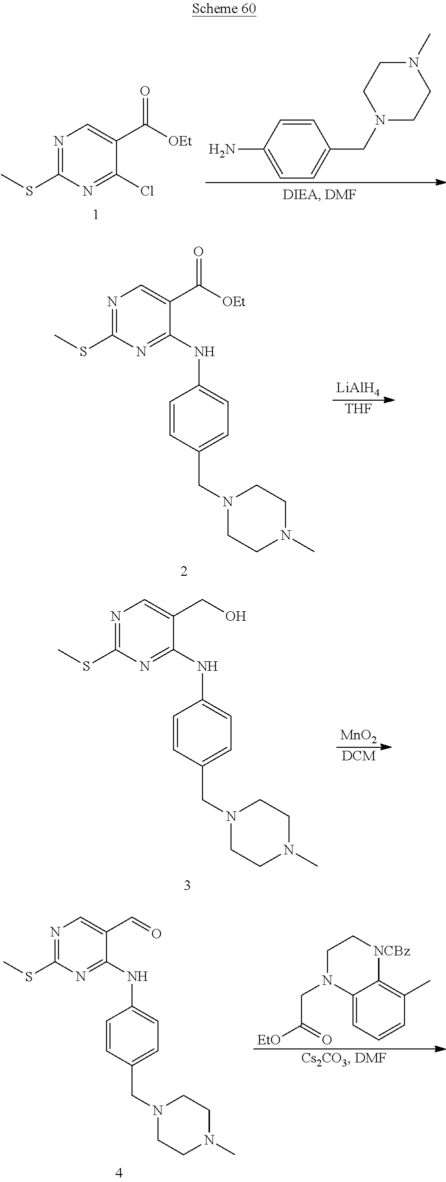

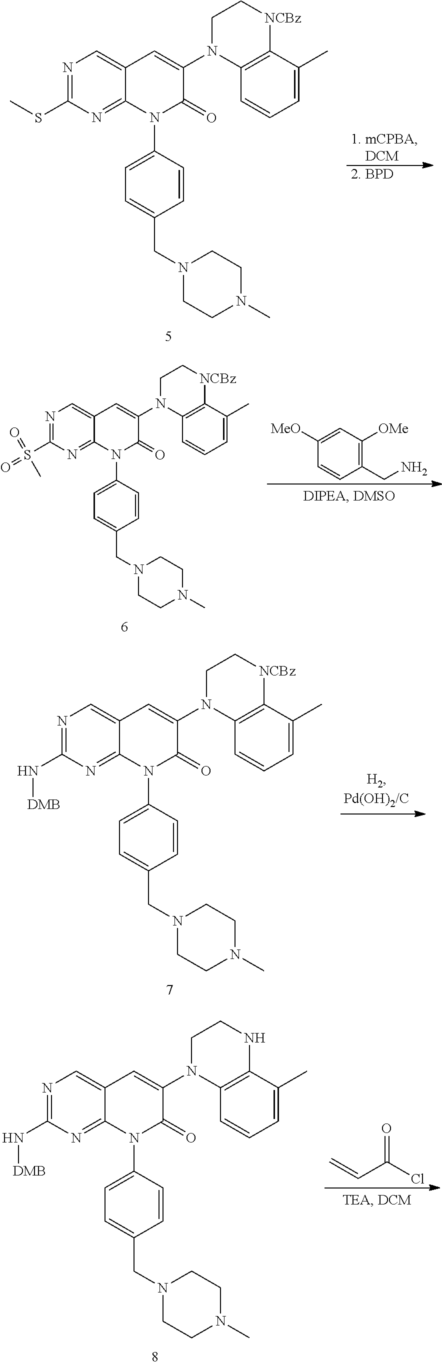

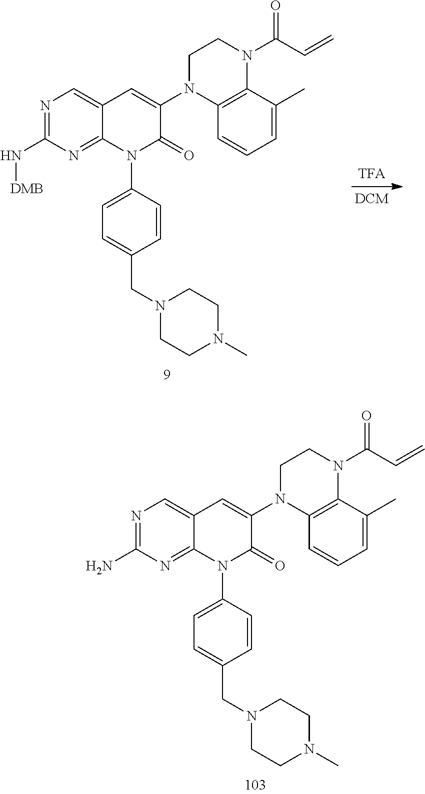

To a solution of 4-[(4-methylpiperazin-1-yl)methyl]aniline (2.6 g, 12.8 mmol, 1 eq) in DMF (30 mL) was added DIEA (5.0 g, 38.6 mmol, 6.7 mL, 3 eq) and ethyl 4-chloro-2-methylsulfanyl-pyrimidine-5-carboxylate (3 g, 12.8 mmol, 1 eq). The mixture was stirred at 20° C. for 12 hr. LC-MS showed desired compound was detected. The reaction mixture was extracted with H₂O (30 mL) and ethyl acetate (20 mL× 3). The combined organic layers were washed with brine (20 mL× 3), dried over Na₂SO₄, filtered, and concentrated under reduced pressure to give ethyl 4-[4-[(4-methylpiperazin-1-yl)methyl]anilino]-2-methylsulfanyl-pyrimidine-5-carboxylate (5 g, crude) as yellow oil.

To a solution of ethyl 4-[4-[(4-methylpiperazin-1-yl)methyl]anilino]-2-methylsulfanyl-pyrimidine-5-carboxylate (5 g, 12.4 mmol, 1 eq) in THF (50 mL) was added LiAlH₄ (2.5 M, 7.4 mL, 1.5 eq) at 0° C. under N₂. The mixture was stirred at 0° C. for 1 hr under N₂ atmosphere. LC-MS showed desired compound was detected. The reaction mixture was quenched by addition of Na₂SO₄10H₂O 10 g at 0° C., filtered, and concentrated under reduced pressure to give [4-[4-[(4-methylpiperazin-1-yl)methyl]anilino]-2-methylsulfanyl-pyrimidin-5-yl]methanol (3.6 g, crude) as a

To a solution of [4-[4-[(4-methylpiperazin-1-yl)methyl]anilino]-2-methylsulfanyl-pyrimidin-5-yl]methanol (3.6 g, 10.1 mmol, 1 eq) in DCM (50 mL) was added MnO₂ (8.8 g, 101·2 mmol, 10 eq). The mixture was stirred at 40° C. for 12 hr. LC-MS showed desired compound was detected. The reaction mixture was filtered and concentrated under reduced pressure to give a residue. The residue was purified by flash silica gel chromatography (20 g Silica Flash Column, Eluent of 0˜75% Ethyl acetate/Petroleum ether gradient @ 100 mL/min) to give 4-[4-[(4-methylpiperazin-1-yl)methyl]anilino]-2-methylsulfanyl-pyrimidine-5-carbaldehyde (1.5 g, 3.0 mmol, 30·2% yield, 73% purity) as a yellow solid.

To a solution of 4-[4-[(4-methylpiperazin-1-yl)methyl]anilino]-2-methylsulfanyl-pyrimidine-5-carbaldehyde (1.5 g, 4·2 mmol, 1 eq) in DMF (40 mL) was added Cs₂CO₃ (4.1 g, 12.5 mmol, 3 eq) and benzyl 4-(2-ethoxy-2-oxo-ethyl)-8-methyl-2,3-dihydroquinoxaline-1-carboxylate (1.5 g, 4·2 mmol, 1 eq). The mixture was stirred at 80° C. for 12 hr. LC-MS showed desired compound was detected. The reaction mixture was extracted with H₂O (30 mL) and ethyl acetate (20 ml×3). The combined organic layers were washed with brine (20 mL× 3), dried over Na₂SO₄, filtered, and concentrated under reduced pressure to give a residue. The residue was purified by flash silica gel chromatography (20 g Silica Flash Column, Eluent of 0˜100% Ethyl acetate/Petroleum ether gradient @ 100 mL/min) to give benzyl 8-methyl-4-[8-[4-[(4-methylpiperazin-1-yl)methyl]phenyl]-2-methylsulfanyl-7-oxo-pyrido[2,3-d]pyrimidin-6-yl]-2,3-dihydroquinoxaline-1-carboxylate (1·2 g, 1.3 mmol, 31.1% yield, 72% purity) as a yellow solid.

To a solution of benzyl 8-methyl-4-[8-[4-[(4-methylpiperazin-1-yl)methyl]phenyl]-2-methylsulfanyl-7-oxo-pyrido[2,3-d]pyrimidin-6-yl]-2,3-dihydroquinoxaline-1-carboxylate (1.1 g, 1.7 mmol, 1 eq) in DCM (50 mL) was added mCPBA (1.0 g, 5·2 mmol, 85% purity, 3 eq) at 0° C. The mixture was stirred at 20° C. for 2 hr. Then the mixture was added BPD (882.5 mg, 3.4 mmol, 2 eq) and stirred at 20° C. for 1 hr. LC-MS showed desired compound was detected. The reaction mixture was quenched by addition Na₂SO₃ (200 mL), and then extracted with dichloromethane (100 mL×3). The combined organic layers were washed with NaHCO₃ (100 mL× 3), dried over Na₂SO₄, filtered and concentrated under reduced pressure to give benzyl 8-methyl-4-[8-[4-[(4-methylpiperazin-1-yl)methyl]phenyl]-2-methylsulfonyl-7-oxo-pyrido[2,3-d]pyrimidin-6-yl]-2,3-dihydroquinoxaline-1-carboxylate (1·2 g, crude) as a orange solid.

To a solution of benzyl 8-methyl-4-[8-[4-[(4-methylpiperazin-1-yl)methyl]phenyl]-2-methylsulfonyl-7-oxo-pyrido[2,3-d]pyrimidin-6-yl]-2,3-dihydroquinoxaline-1-carboxylate (1.1 g, 1.5 mmol, 1 eq) in DMSO (11 mL) was added DIPEA (614.7 mg, 4.7 mmol, 828.4 μL, 3 eq) and (2,4-dimethoxyphenyl) methanamine (265.0 mg, 1.5 mmol, 238.1 μL, 1 eq). The mixture was stirred at 20° C. for 2 hr. LC-MS showed desired compound was detected. The reaction mixture was extracted with H₂O (30 mL) and ethyl acetate (20 mL×3). The combined organic layers were washed with brine (20 mL× 3), dried over Na₂SO₄, filtered and concentrated under reduced pressure to give a residue. The residue was purified by prep-HPLC (TFA condition column: Phenomenex luna C18 100×40 mm×3 μm; mobile phase: [H₂O (0.1% TFA)-ACN]; gradient: 35%-65% B over 8.0 min) to give benzyl 4-[2-[(2,4-dimethoxyphenyl)methylamino]-8-[4-[(4-methylpiperazin-1-yl)methyl]phenyl]-7-oxo-pyrido[2,3-d]pyrimidin-6-yl]-8-methyl-2,3-dihydroquinoxaline-1-carboxylate (720 mg, 912.7 μmol, 57.5% yield, 99% purity) as a yellow solid.

To a solution of Pd(OH)₂/C (355 mg, 20% purity) in MeOH (20 mL) was added benzyl 4-[2-[(2,4-dimethoxyphenyl)methylamino]-8-[4-[(4-methylpiperazin-1-yl)methyl]phenyl]-7-oxo-pyrido[2,3-d]pyrimidin-6-yl]-8-methyl-2,3-dihydroquinoxaline-1-carboxylate (710 mg, 909.1 μmol, 1 eq) under N₂ atmosphere. The suspension was degassed and purged with H₂ for 3 times. The mixture was stirred under H₂ (15 Psi.) at 20° C. for 2 hr. LC-MS showed desired compound was detected. The reaction mixture was filtered and concentrated under reduced pressure to give 2-[(2,4-dimethoxyphenyl)methylamino]-6-(5-methyl-3,4-dihydro-2H-quinoxalin-1-yl)-8-[4-[(4-methylpiperazin-1-yl)methyl]phenyl]pyrido[2,3-d]pyrimidin-7-one (700 mg, crude) as yellow oil.

At 0° C., to a solution of 2-[(2,4-dimethoxyphenyl)methylamino]-6-(5-methyl-3,4-dihydro-2H-quinoxalin-1-yl)-8-[4-[(4-methylpiperazin-1-yl)methyl]phenyl]pyrido[2,3-d]pyrimidin-7-one (100 mg, 154.6 μmol, 1 eq) in DCM (2 mL) was added TEA (46.9 mg, 463.8 μmol, 64.5 μL, 3 eq) and prop-2-enoyl chloride (27.9 mg, 309·2 μmol, 25.1 μL, 2 eq). The mixture was stirred at 0° C. for 30 min (batch× 2). LC-MS showed desired compound was detected. The reaction mixture was concentrated under N₂ to give 2-[(2,4-dimethoxyphenyl)methylamino]-8-[4-[(4-methylpiperazin-1-yl)methyl]phenyl]-6-(5-methyl-4-prop-2-enoyl-2,3-dihydroquinoxalin-1-yl)pyrido[2,3-d]pyrimidin-7-one (120 mg, crude) as a yellow solid.

To a solution of 2-[(2,4-dimethoxyphenyl)methylamino]-8-[4-[(4-methylpiperazin-1-yl)methyl]phenyl]-6-(5-methyl-4-prop-2-enoyl-2,3-dihydroquinoxalin-1-yl)pyrido[2,3-d]pyrimidin-7-one (240 mg, 342.4 μmol, 1 eq) in DCM (2 mL) was added TFA (1.8 g, 16.1 mmol, 1·2 mL, 47.1 eq). The mixture was stirred at 20° C. for 12 hr. LC-MS showed desired compound was detected. The reaction mixture was concentrated under reduced pressure to remove solvent. The residue was purified by prep-HPLC (TFA condition: column: Phenomenex luna C18 100× 40 mm× 3 μm; mobile phase: [H₂O (0.1% TFA)-ACN]; gradient: 10%-40% B over 8.0 min) to give 2-amino-8-[4-[(4-methylpiperazin-1-yl)methyl]phenyl]-6-(5-methyl-4-prop-2-enoyl-2,3-dihydroquinoxalin-1-yl)pyrido[2,3-d]pyrimidin-7-one (25.9 mg, 47.0 μmol, 13.7% yield, 100% purity) as a yellow solid.

### Procedure for Preparation of Compound 090

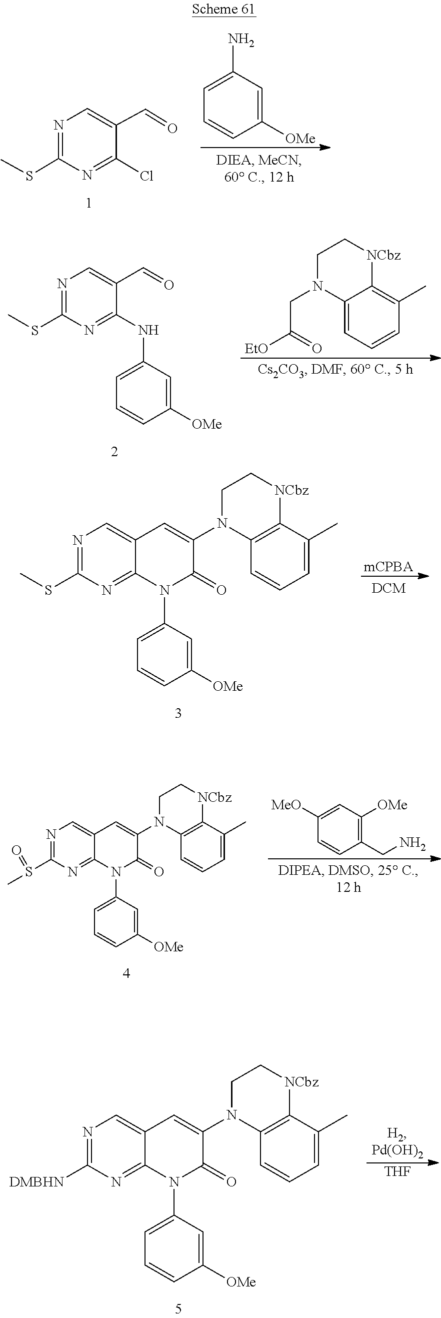

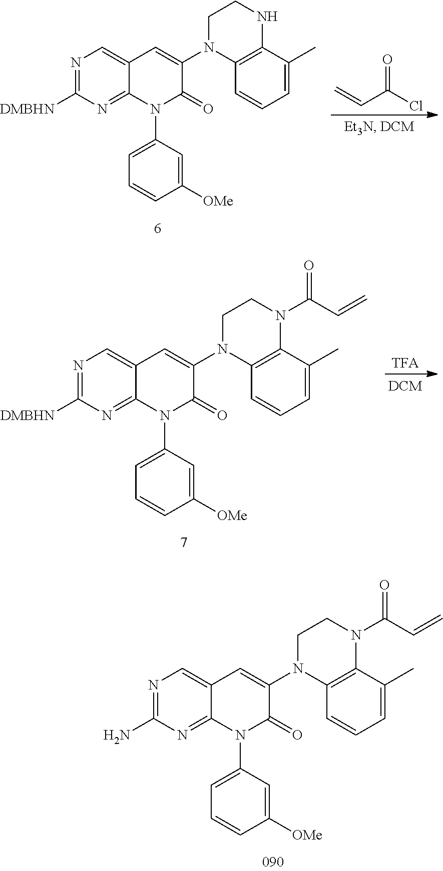

To a solution of 4-chloro-2-methylsulfanyl-pyrimidine-5-carbaldehyde (1.3 g, 6.6 mmol, 1 eq) in MeCN (20 mL) was added DIEA (2.6 g, 19.9 mmol, 3.5 mL, 3 eq) and 3-methoxyaniline (816.1 mg, 6.6 mmol, 744.6 μL, 1 eq). The mixture was stirred at 60° C. for 12 hr (batch×2). LC-MS showed desired compound was detected. The reaction was concentrated under reduce pressure to give a residue. The residue was purified by flash silica gel chromatography (12 g Silica Flash Column, Eluent of 0˜100% Ethyl acetate/Petroleum ether gradient @ 80 mL/min) to give compound 4-(3-methoxyanilino)-2-methylsulfanyl-pyrimidine-5-carbaldehyde (2.5 g, 9·2 mmol, 69.3% yield) as a yellow solid.

To a solution of 4-(3-methoxyanilino)-2-methylsulfanyl-pyrimidine-5-carbaldehyde (2.5 g, 9·2 mmol, 1 eq) in DMF (40 mL) was added benzyl 4-(2-ethoxy-2-oxo-ethyl)-8-methyl-2,3-dihydroquinoxaline-1-carboxylate (3.7 g, 10.1 mmol, 1.1 eq) and Cs₂CO₃ (9.0 g, 27.6 mmol, 3 eq). The mixture was stirred at 60° C. for 5 hr. LC-MS showed desired compound was detected. The reaction mixture was concentrated under reduced pressure to remove solvent. The residue was diluted with H₂O (15 mL) and extracted with ethyl acetate (20 mL×3). The combined organic layers were dried over Na₂SO₄, filtered and concentrated under reduced pressure to give a residue. The residue was purified by flash silica gel chromatography (20 g Silica Flash Column, Eluent of 0˜100% Ethyl acetate/Petroleum ether gradient @ 80 mL/min) to give compound benzyl 4-[8-(3-methoxyphenyl)-2-methylsulfanyl-7-oxo-pyrido[2,3-d]pyrimidin-6-yl]-8-methyl-2,3-dihydroquinoxaline-1-carboxylate (3.1 g, 5.3 mmol, 28.8% yield) as a yellow solid.

To a solution of benzyl 4-[8-(3-methoxyphenyl)-2-methylsulfanyl-7-oxo-pyrido[2,3-d]pyrimidin-6-yl]-8-methyl-2,3-dihydroquinoxaline-1-carboxylate (3.1 g, 5.3 mmol, 1 eq) in DCM (50 mL) was added mCPBA (1.3 g, 6.4 mmol, 85% purity, 1·2 eq) at 0° C. The mixture was stirred at 25° C. for 2 hr. LC-MS showed desired compound was detected. The reaction mixture was quenched by addition sat. Na₂SO₃ (20 mL) at 25° C., then diluted with dichloromethane (15 mL) and extracted with dichloromethane (10 mL× 3) and washed with sat. NaHCO₃ (10 mL× 3), dried over Na₂SO₄, filtered and concentrated under reduced pressure to give compound benzyl 4-[8-(3-methoxyphenyl)-2-methylsulfinyl-7-oxo-pyrido[2,3-d]pyrimidin-6-yl]-8-methyl-2,3-dihydroquinoxaline-1-carboxylate (2.8 g, crude) as a yellow solid.

To a solution of benzyl 4-[8-(3-methoxyphenyl)-2-methylsulfinyl-7-oxo-pyrido[2,3-d]pyrimidin-6-yl]-8-methyl-2,3-dihydroquinoxaline-1-carboxylate (800 mg, 1.3 mmol, 1 eq) in DMSO (10 mL) was added (2,4-dimethoxyphenyl) methanamine (269.5 mg, 1.6 mmol, 242.1 μL, 1·2 eq) and DIEA (520.7 mg, 4.0 mmol, 701.8 μL, 3 eq). The mixture was stirred at 25° C. for 12 hr. LC-MS showed desired compound was detected. The residue was diluted with H₂O (20 mL) and extracted with ethyl acetate (20 mL× 3). The combined organic layers were dried over Na₂SO₄, filtered and concentrated under reduced pressure to give a residue. The residue was purified by flash silica gel chromatography (12 g Silica Flash Column, Eluent of 0˜100% Ethyl acetate/Petroleum ether gradient @ 80 mL/min) to give compound benzyl 4-[2-[(2,4-dimethoxyphenyl)methylamino]-8-(3-methoxyphenyl)-7-oxo-pyrido[2,3-d]pyrimidin-6-yl]-8-methyl-2,3-dihydroquinoxaline-1-carboxylate (677 mg, 968.9 μmol, 72.1% yield) as a yellow

To a solution of Pd(OH)₂ (100 mg, 20% purity) in THF (5 mL) was added benzyl 4-[2-[(2,4-dimethoxyphenyl)methylamino]-8-(3-methoxyphenyl)-7-oxo-pyrido[2,3-d]pyrimidin-6-yl]-8-methyl-2,3-dihydroquinoxaline-1-carboxylate (211 mg, 301.9 μmol, 1 eq) under N₂ atmosphere. The suspension was degassed and purged with H₂ for 3 times. The mixture was stirred under H₂ (15 Psi) at 25° C. for 0.5 hr. LC-MS showed desired compound was detected. The reaction was filtered and concentrated under reduced pressure to give compound 2-[(2,4-dimethoxyphenyl)methylamino]-8-(3-methoxyphenyl)-6-(5-methyl-3,4-dihydro-2H-quinoxalin-1-yl)pyrido[2,3-d]pyrimidin-7-one (183 mg, crude) as a yellow oil.

To a solution of 2-[(2,4-dimethoxyphenyl)methylamino]-8-(3-methoxyphenyl)-6-(5-methyl-3,4-dihydro-2H-quinoxalin-1-yl)pyrido[2,3-d]pyrimidin-7-one (170 mg, 301.1 μmol, 1 eq) in DCM (2 mL) was added Et3N to adjust the pH to 7-8, then Et3N (91.4 mg, 903·2 μmol, 125.7 μL, 3 eq) and prop-2-enoyl chloride (21.8 mg, 240.9 μmol, 19.6 μL, 0.8 eq) was added to the mixture at 0° C. The mixture was stirred at 0° C. for 0.5 hr. LC-MS: showed 27% of reactant 1 was remained, prop-2-enoyl chloride (5.5 mg, 60·2 μmol, 4.9 μL, 0·2 eq) was added to the mixture at 0° C., the mixture was stirred at 0° C. for 0.5 hr. LC-MS showed 20% of reactant 1 was remained, prop-2-enoyl chloride (5.5 mg, 60·2 μmol, 4.9 μL, 0·2 eq) was added to the mixture at 0° C., the mixture was stirred at 0° C. for 1 hr. LC-MS showed desired compound was detected. The reaction was concentrated under reduce pressure to give residue. The residue was purified by prep-HPLC (TFA condition; column: Phenomenex luna C18 100×40 mm×3 μm; mobile phase: [H₂O (0.1% TFA)-ACN]; gradient: 45%-75% B over 8.0 min) to give compound 2-[(2,4-dimethoxyphenyl)methylamino]-8-(3-methoxyphenyl)-6-(5-methyl-4-prop-2-enoyl-2,3-dihydroquinoxalin-1-yl)pyrido[2,3-d]pyrimidin-7-one (77 mg, 124.5 μmol, 41.3% yield) as a yellow solid.

To a solution of 2-[(2,4-dimethoxyphenyl)methylamino]-8-(3-methoxyphenyl)-6-(5-methyl-4-prop-2-enoyl-2,3-dihydroquinoxalin-1-yl)pyrido[2,3-d]pyrimidin-7-one (30 mg, 48.5 μmol, 1 eq) in DCM (1 mL) was added TFA (812.7 mg, 7.1 mmol, 529.4 μL, 146.9 eq). The mixture was stirred at 25° C. for 12 hr. LC-MS showed desired compound was detected. The reaction was concentrated under reduce pressure to give residue. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min). to give compound 2-amino-8-(3-methoxyphenyl)-6-(5-methyl-4-prop-2-enoyl-2,3-dihydroquinoxalin-1-yl)pyrido[2,3-d]pyrimidin-7-one (10 mg, 21·2 μmol, 43.8% yield, 99.4% purity) as a white solid.

### Procedure for Preparation of Compound 091

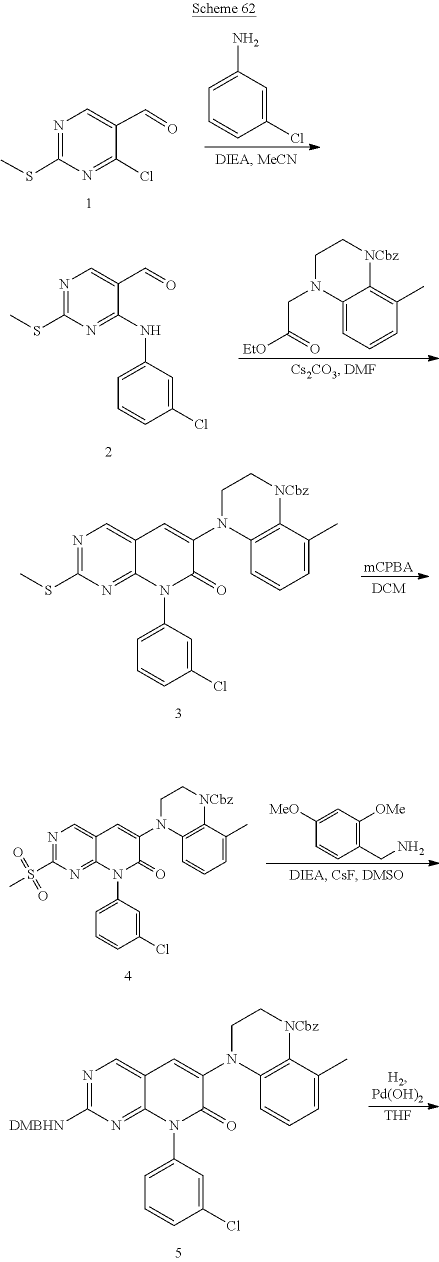

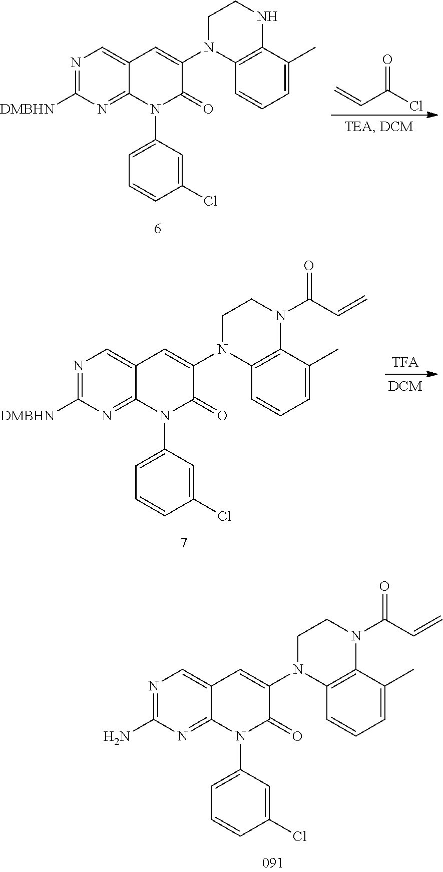

To a solution of 4-chloro-2-methylsulfanyl-pyrimidine-5-carbaldehyde (2 g, 10.6 mmol, 1 eq) in MeCN (20 mL) was added DIEA (4.1 g, 31.8 mmol, 5.5 mL, 3 eq) and 3-chloroaniline (1.4 g, 10.6 mmol, 1.1 mL, 1 eq). The mixture was stirred at 60° C. for 2.5 hr. LC-MS showed desired compound was detected. The reaction mixture was concentrated under reduced pressure to remove solvent. The residue was purified by flash silica gel chromatography (20 g Silica Flash Column, Eluent of 0˜7% Ethyl acetate/Petroleum ether gradient @ 100 mL/min) to give 4-(3-chloroanilino)-2-methylsulfanyl-pyrimidine-5-carbaldehyde (1.3 g, 4.6 mmol, 43·2% yield) as a yellow solid.

To a solution of 4-(3-chloroanilino)-2-methylsulfanyl-pyrimidine-5-carbaldehyde (1.3 g, 4.6 mmol, 1 eq) in DMF (30 mL) was added benzyl 4-(2-ethoxy-2-oxo-ethyl)-8-methyl-2,3-dihydroquinoxaline-1-carboxylate (1.7 g, 4.6 mmol, 1 eq) and Cs₂CO₃ (4.5 g, 13.7 mmol, 3 eq). The mixture was stirred at 60° C. for 14 hr. LC-MS showed desired compound was detected. The residue was diluted with H₂O (100 mL) and extracted with ethyl acetate (120 mL×4). The combined organic layers were washed with brine (100 mL×3), dried over Na₂SO₄, filtered and concentrated under reduced pressure to give a residue. The residue was purified by flash silica gel chromatography (20 g Silica Flash Column, Eluent of 0˜20% Ethyl acetate/Petroleum ether gradient @ 120 mL/min) to give benzyl 4-[8-(3-chlorophenyl)-2-methylsulfanyl-7-oxo-pyrido[2,3-d]pyrimidin-6-yl]-8-methyl-2,3-dihydroquinoxaline-1-carboxylate (800 mg, 1.4 mmol, 29.9% yield) as a yellow solid.

To a solution of benzyl 4-[8-(3-chlorophenyl)-2-methylsulfanyl-7-oxo-pyrido[2,3-d]pyrimidin-6-yl]-8-methyl-2,3-dihydroquinoxaline-1-carboxylate (800 mg, 1.4 mmol, 1 eq) in DCM (10 mL) was added mCPBA (417.1 mg, 2.1 mmol, 85% purity, 1.5 eq) at 0° C. The mixture was stirred at 20° C. for 2 hr. LC-MS showed desired compound was detected. The residue was diluted with Na₂SO₃ (40 mL) and extracted with dichloromethane (40 mL× 3). The combined organic layers were washed with NaHCO₃ (30 mL× 3), dried over Na₂SO₄, filtered and concentrated under reduced pressure to give benzyl 4-[8-(3-chlorophenyl)-2-methylsulfonyl-7-oxo-pyrido[2,3-d]pyrimidin-6-yl]-8-methyl-2,3-dihydroquinoxaline-1-carboxylate (800 mg, crude) as an orange solid.

To a solution of benzyl 4-[8-(3-chlorophenyl)-2-methylsulfonyl-7-oxo-pyrido[2,3-d]pyrimidin-6-yl]-8-methyl-2,3-dihydroquinoxaline-1-carboxylate (360 mg, 584.3 μmol, 1 eq) in DMSO (4 mL) was added (2,4-dimethoxyphenyl) methanamine (117·2 mg, 701·2 μmol, 105.3 μL, 1·2 eq), DIPEA (226.6 mg, 1.8 mmol, 305.3 μL, 3 eq) and CsF (88.8 mg, 584.3 μmol, 21.6 μL, 1 eq). The mixture was stirred at 20° C. for 1 hr. LC-MS showed desired compound was detected. The residue was diluted with H₂O (10 mL) and extracted with ethyl acetate (15 mL×4). The combined organic layers were washed with brine (10 mL× 3), dried over Na₂SO₄, filtered and concentrated under reduced pressure to give a residue. The residue was purified by flash silica gel chromatography (4 g Silica Flash Column, Eluent of 0˜50% Ethyl acetate/Petroleum ether gradient @ 80 mL/min) to give benzyl 4-[8-(3-chlorophenyl)-2-[(2,4-dimethoxyphenyl)methylamino]-7-oxo-pyrido[2,3-d]pyrimidin-6-yl]-8-methyl-2,3-dihydroquinoxaline-1-carboxylate (330 mg, 469.3 μmol, 80.3% yield) as a yellow solid.

To a solution of benzyl 4-[8-(3-chlorophenyl)-2-[(2,4-dimethoxyphenyl)methylamino]-7-oxo-pyrido[2,3-d]pyrimidin-6-yl]-8-methyl-2,3-dihydroquinoxaline-1-carboxylate (230 mg, 327.1 μmol, 1 eq) in THF (10 mL) was added TEA to PH=8, Pd(OH)₂ (120 mg, 20% purity) under N₂. The suspension was degassed under vacuum and purged with H₂ several times. The mixture was stirred under H₂ (15 psi) at 20° C. for 1 hr. LC-MS showed desired compound was detected. The reaction mixture filtered and concentrated under reduced pressure to give 8-(3-chlorophenyl)-2-[(2,4-dimethoxyphenyl)methylamino]-6-(5-methyl-3,4-dihydro-2H-quinoxalin-1-yl)pyrido[2,3-d]pyrimidin-7-one (50 mg, crude) as a yellow oil.

To a solution of 8-(3-chlorophenyl)-2-[(2,4-dimethoxyphenyl)methylamino]-6-(5-methyl-3,4-dihydro-2H-quinoxalin-1-yl)pyrido[2,3-d]pyrimidin-7-one (100 mg, 175.7 μmol, 1 eq) in DCM (3 mL) was added TEA (53.4 mg, 527·2 μmol, 73.4 μL, 3 eq) and prop-2-enoyl chloride (15.9 mg, 175.7 μmol, 14.3 μL, 1 eq) at 0° C. The mixture was stirred at 0° C. for 0.5 hr. LC-MS showed desired compound was detected. The reaction mixture was concentrated under reduced pressure to give 8-(3-chlorophenyl)-2-[(2,4-dimethoxyphenyl)methylamino]-6-(5-methyl-4-prop-2-enoyl-2,3-dihydroquinoxalin-1-yl)pyrido[2,3-d]pyrimidin-7-one (100 mg, crude) as a yellow solid.

A mixture of 8-(3-chlorophenyl)-2-[(2,4-dimethoxyphenyl)methylamino]-6-(5-methyl-4-prop-2-enoyl-2,3-dihydroquinoxalin-1-yl)pyrido[2,3-d]pyrimidin-7-one (100 mg, 160.5 μmol, 1 eq) in DCM (2 mL) and TFA (0.6 mL) was stirred at 25° C. for 2 hr. LC-MS showed desired compound was detected. The reaction mixture was concentrated under reduced pressure to remove solvent. The residue was purified by prep-HPLC (TFA condition. column: Phenomenex luna C18 100×40 mm×3 μm; mobile phase: [H₂O (0.1% TFA)-ACN]; gradient: 25%-55% B over 8.0 min) to give 2-amino-8-(3-chlorophenyl)-6-(5-methyl-4-prop-2-enoyl-2,3-dihydroquinoxalin-1-yl)pyrido[2,3-d]pyrimidin-7-one (14.18 mg, 29.98 μmol, 18.68% yield, 100% purity) as a yellow solid.

### Procedure for Preparation of Compound 099

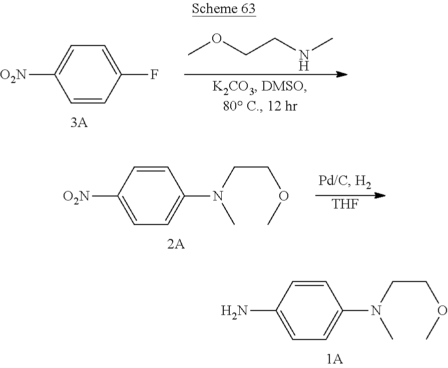

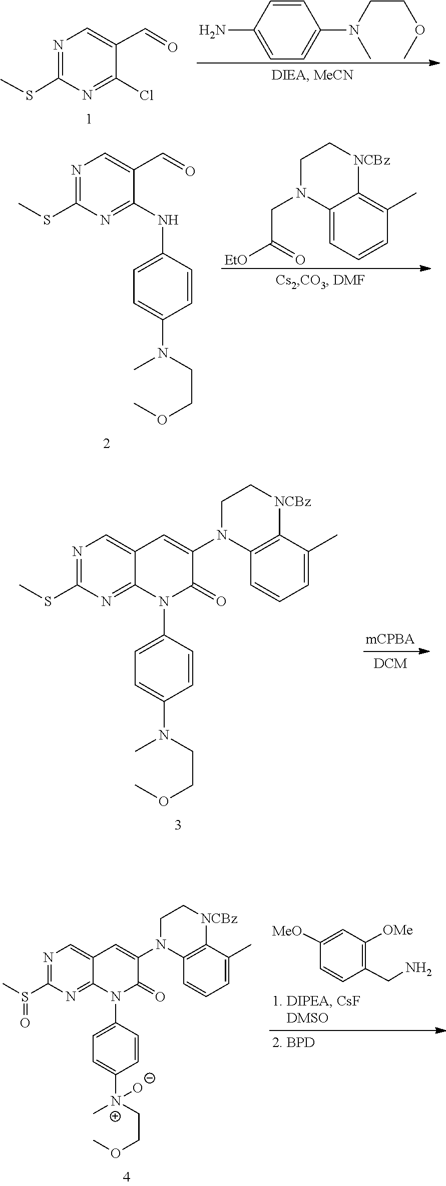

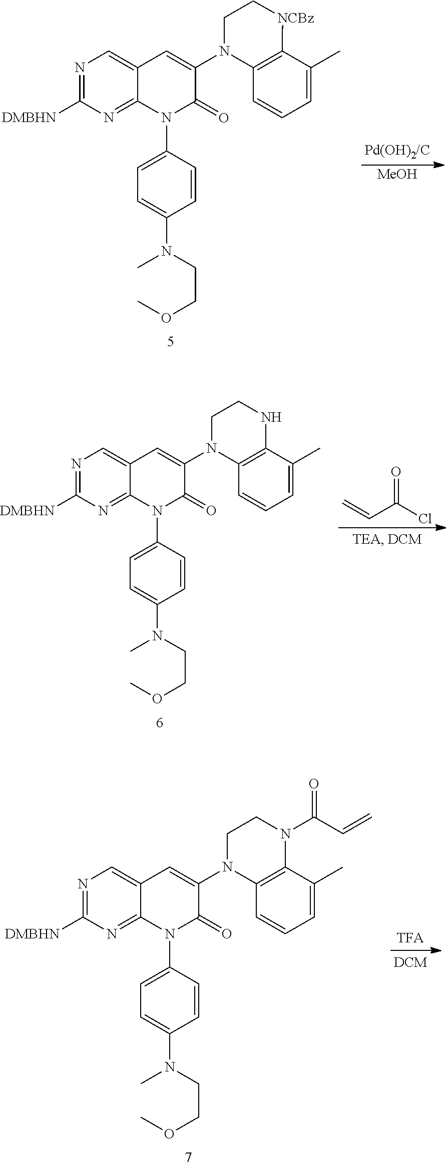

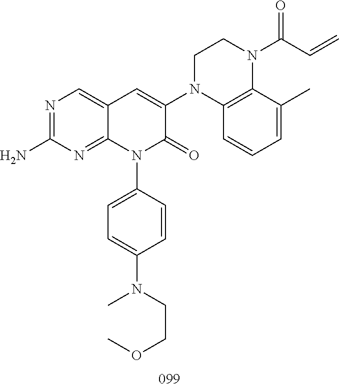 [SMILES: C=CC(=O)N1CCN(c2cc3cnc(N)nc3n(-c3ccc(N(C)CCOC)cc3)c2=O)c2cccc(C)c21]

To a solution of 1-fluoro-4-nitro-benzene (4 g, 28.4 mmol, 3.0 mL, 1 eq) in DMSO (40 mL) was added K₂CO₃ (11.8 g, 85.0 mmol, 3 eq) and 2-methoxy-N-methyl-ethanamine (3.8 g, 42.5 mmol, 4.6 mL, 1.5 eq). The mixture was stirred at 80° C. for 12 hr. LC-MS showed desired compound was detected. The residue was diluted with H₂O (60 mL) and extracted with ethyl acetate (60 mL× 3). The combined organic layers were washed with brine (40 mL× 1), dried over Na₂SO₄, filtered and concentrated under reduced pressure to give N-(2-methoxyethyl)-N-methyl-4-nitro-aniline (5.9 g, crude) as a yellow solid.

To a solution of Pd/C (3 g, 10% purity) in THF (50 mL) was added N-(2-methoxyethyl)-N-methyl-4-nitro-aniline (5.9 g, 28.1 mmol, 1 eq) under N₂ atmosphere. The suspension was degassed and purged with H₂ for 3 times. The mixture was stirred under H₂ (15 psi) at 20° C. for 12 hr. LC-MS showed desired compound was detected. The reaction mixture was concentrated under reduced pressure to give N1-(2-methoxyethyl)-N1-methyl-benzene-1,4-diamine (5.1 g, crude) as a purple solid.

To a solution of 4-chloro-2-methylsulfanyl-pyrimidine-5-carbaldehyde (500 mg, 2.7 mmol, 1 eq) in MeCN (10 mL) was added DIEA (1.0 g, 8.0 mmol, 1.4 mL, 3 eq) and N1-(2-methoxyethyl)-N1-methyl-benzene-1,4-diamine (477.8 mg, 2.7 mmol, 1 eq). The mixture was stirred at 20° C. for 12 hr. LC-MS showed desired compound was detected. The reaction mixture was concentrated under reduced pressure to remove solvent. The residue was purified by flash silica gel chromatography (12 g Silica Flash Column, Eluent of 0˜15% Ethyl acetate/Petroleum ether gradient @ 100 mL/min) to give 4-[4-[2-methoxyethyl(methyl)amino]anilino]-2-methylsulfanyl-pyrimidine-5-carbaldehyde (700 mg, 2.1 mmol, 79.4% yield) as an orange solid.

To a solution of 4-[4-[2-methoxyethyl(methyl)amino]anilino]-2-methylsulfanyl-pyrimidine-5-carbaldehyde (600 mg, 1.80 mmol, 1 eq) in DMF (20 mL) was added Cs₂CO₃ (1.76 g, 5.41 mmol, 3 eq) and benzyl 4-(2-ethoxy-2-oxo-ethyl)-8-methyl-2,3-dihydroquinoxaline-1-carboxylate (665.0 mg, 1.80 mmol, 1 eq). The mixture was stirred at 60° C. for 2 hr. LC-MS showed desired compound was detected. The residue was diluted with H₂O (50 mL) and extracted with ethyl acetate (50 mL× 3). The combined organic layers were washed with brine (20 mL× 2), dried over Na₂SO₄, filtered and concentrated under reduced pressure to give a residue. The residue was purified by flash silica gel chromatography (12 g Silica Flash Column, Eluent of 0˜24% Ethyl acetate/Petroleum ether gradient @100 mL/min) to give benzyl 4-[8-[4-[2-methoxyethyl(methyl)amino]phenyl]-2-methylsulfanyl-7-oxo-pyrido[2,3-d]pyrimidin-6-yl]-8-methyl-2,3-dihydroquinoxaline-1-carboxylate (780 mg, 1·2 mmol, 67.9% yield) as a yellow solid.

To a solution of benzyl 4-[8-[4-[2-methoxyethyl(methyl)amino]phenyl]-2-methylsulfanyl-7-oxo-pyrido[2,3-d]pyrimidin-6-yl]-8-methyl-2,3-dihydroquinoxaline-1-carboxylate (600 mg, 942.3 μmol, 1 eq) in DCM (10 mL) was added mCPBA (478·2 mg, 2.4 mmol, 85% purity, 2.5 eq) at 0° C. The mixture was stirred at 0° C. for 3 hr. LC-MS showed desired compound was detected. The residue was diluted with Na₂SO₃ (150 mL) and extracted with DCM (150 mL×3). The combined organic layers were washed with NaHCO₃ (100 mL× 1), dried over Na₂SO₄, filtered and concentrated under reduced pressure to give 4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-(2-methoxyethyl)-N-methyl-benzeneamine oxide (670 mg, crude) as an orange solid.

To a solution of 4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-(2-methoxyethyl)-N-methyl-benzeneamine oxide (250 mg, 373.8 μmol, 1 eq) in DMSO (2.5 mL) was added (2,4-dimethoxyphenyl) methanamine (93.8 mg, 560.7 μmol, 84·2 μL, 1.5 eq), DIPEA (144.9 mg, 1.1 mmol, 195.3 μL, 3 eq) and CsF (56.8 mg, 373.8 μmol, 13.8 μL, 1 eq) (batch× 2). The mixture was stirred at 20° C. for 1 hr. Then BPD (284.8 mg, 1.1 mmol, 3 eq) was added to the mixture. The mixture was stirred at 20° C. for 1 hr. LC-MS showed desired compound was detected. The residue was diluted with H₂O 20 mL and extracted with ethyl acetate (20 mL×3). The residue was purified by flash silica gel chromatography (12 g Silica Flash Column, Eluent of 0˜45% Ethyl acetate/Petroleum ether gradient @ 100 mL/min) to give benzyl 4-[2-[(2,4-dimethoxyphenyl)methylamino]-8-[4-[2-methoxyethyl(methyl)amino]phenyl]-7-oxo-pyrido[2,3-d]pyrimidin-6-yl]-8-methyl-2,3-dihydroquinoxaline-1-carboxylate (390 mg, 516.0 μmol, 69.0% yield) as a yellow solid.

To a solution of Pd(OH)₂/C (100 mg, 20% purity) in MeOH (5 mL) was added benzyl 4-[2-[(2,4-dimethoxyphenyl)methylamino]-8-[4-[2-methoxyethyl(methyl)amino]phenyl]-7-oxo-pyrido[2,3-d]pyrimidin-6-yl]-8-methyl-2,3-dihydroquinoxaline-1-carboxylate (170 mg, 224.9 μmol, 1 eq) under N₂ atmosphere. The suspension was degassed and purged with H₂ for 3 times. The mixture was stirred under H₂ (15 psi) at 20° C. for 1 hr. LC-MS showed desired compound was detected. The reaction mixture was filtered and concentrated under reduced pressure to give 2-[(2,4-dimethoxyphenyl)methylamino]-8-[4-[2-methoxyethyl(methyl)amino]phenyl]-6-(5-methyl-3,4-dihydro-2H-quinoxalin-1-yl)pyrido[2,3-d]pyrimidin-7-one (139 mg, crude) as a yellow solid.

To a solution of 2-[(2,4-dimethoxyphenyl)methylamino]-8-[4-[2-methoxyethyl(methyl)amino]phenyl]-6-(5-methyl-3,4-dihydro-2H-quinoxalin-1-yl)pyrido[2,3-d]pyrimidin-7-one (99 mg, 159·2 μmol, 1 eq) in DCM (1 mL) was added TEA (48.3 mg, 477.7 μmol, 66.5 μL, 3 eq) and prop-2-enoyl chloride (14.4 mg, 159·2 μmol, 13.0 μL, 1 eq) at 0° C. The mixture was stirred at 0° C. for 1 hr. LC-MS showed desired compound was detected. The reaction mixture was concentrated under N₂ to remove solvent to give 2-[(2,4-dimethoxyphenyl)methylamino]-8-[4-[2-methoxyethyl(methyl)amino]phenyl]-6-(5-methyl-4-prop-2-enoyl-2,3-dihydroquinoxalin-1-yl)pyrido[2,3-d]pyrimidin-7-one (100 mg, crude) as yellow solid used into the next step without further purification.

To a solution of 2-[(2,4-dimethoxyphenyl)methylamino]-8-[4-[2-methoxyethyl(methyl)amino]phenyl]-6-(5-methyl-4-prop-2-enoyl-2,3-dihydroquinoxalin-1-yl)pyrido[2,3-d]pyrimidin-7-one (100 mg, 148.0 μmol, 1 eq) in DCM (1 mL) was added TFA (0·2 mL). The mixture was stirred at 20° C. for 12 hr. LC-MS showed desired compound was detected. The reaction mixture was concentrated under reduced pressure to remove solvent. The residue was purified by prep-HPLC (column; Phenomenex luna C18 80×40 mm×3 μm; mobile phase: [H₂O (0.1% TFA)-ACN]; gradient: 23%-53% B over 8.0 min) to give 2-amino-8-[4-[2-methoxyethyl(methyl)amino]phenyl]-6-(5-methyl-4-prop-2-enoyl-2,3-dihydroquinoxalin-1-yl)pyrido[2,3-d]pyrimidin-7-one (39·2 mg, 73.8 μmol, 49.9% yield, 99.1% purity) as a white solid.

### Procedure for Preparation of Compound 095

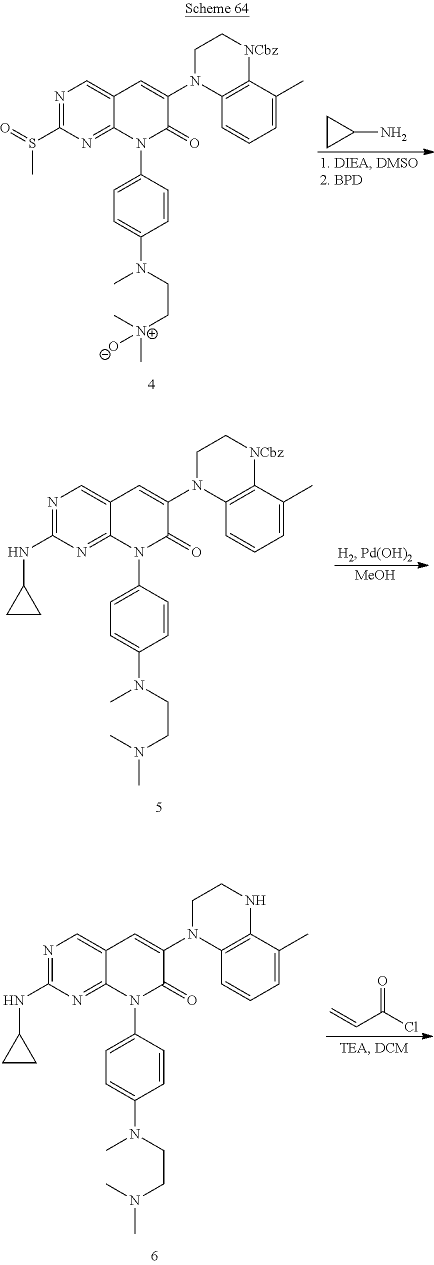

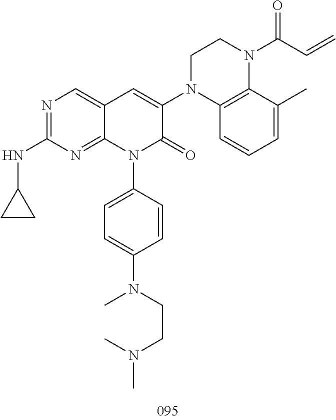 [SMILES: C=CC(=O)N1CCN(c2cc3cnc(NC4CC4)nc3n(-c3ccc(N(C)CCN(C)C)cc3)c2=O)c2cccc(C)c21]

To a solution of 2-[4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-methyl-anilino]—N, N-dimethyl-ethanamine oxide (260 mg, 381.3 μmol, 1 eq) in DMSO (3 mL) was added DIEA (246.4 mg, 1.9 mmol, 332.1 μL, 5 eq) and cyclopropanamine (32.7 mg, 572.0 μmol, 39.6 μL, 1.5 eq). The mixture was stirred at 20° C. for 1 hr. Then added BPD (290.5 mg, 1.1 mmol, 3 eq) to the reaction mixture. The mixture was stirred at 20° C. for 12 hr. LC-MS showed desired compound was detected. The reaction mixture was partitioned between H₂O (30 mL) and ethyl acetate (30 mL×3). The organic phase was separated, washed with brine (30 mL×2), dried over Na₂SO₄, filtered and concentrated under reduced pressure to give a residue. The residue was purified by prep-TLC (SiO₂, DCM: MeOH=5:1) to give benzyl 4-[2-(cyclopropylamino)-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (100 mg, 151.8 μmol, 39.8% yield) as a yellow solid.

To a solution of Pd(OH)₂ (30 mg, 20% purity) in MeOH (2 mL) was added benzyl 4-[2-(cyclopropylamino)-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (60 mg, 91.1 μmol, 1 eq) and TEA (18.4 mg, 182·2 μmol, 25.4 μL, 2 eq) under N₂ atmosphere. The suspension was degassed and purged with H₂ for 3 times. The mixture was stirred under H₂ 15 Psi at 20° C. for 1.5 hr. LC-MS showed desired compound was detected. The reaction mixture was filtered, and the filtrate dried in vacuum to give 2-(cyclopropylamino)-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (45 mg, crude) as a yellow solid.

To a solution of 2-(cyclopropylamino)-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (60 mg, 114.4 μmol, 1 eq) in DCM (1 mL) was added TEA (34.7 mg, 343.1 μmol, 47.8 μL, 3 eq) and prop-2-enoyl chloride (10.4 mg, 114.4 μmol, 9.3 μL, 1 eq) at 0° C. The mixture was stirred at 0° C. for 15 min. LC-MS showed desired compound was detected. The reaction mixture was concentrated under reduced pressure to remove solvent. The residue was purified by prep-HPLC (TFA condition column: Phenomenex luna C18 100×40 mm× 3 μm; mobile phase: [H₂O (0.1% TFA)-ACN]; gradient: 30%-60% B over 8.0 min) to give 2-(cyclopropylamino)-8-[4-[2-(dimethylamino)ethyl-methyl-amino]phenyl]-6-(5-methyl-4-prop-2-enoyl-2,3-dihydroquinoxalin-1-yl)pyrido[2,3-d]pyrimidin-7-one (35.5 mg, 50.6 μmol, 44.3% yield, 98.7% purity, TFA) as a yellow solid.

### Procedure for Preparation of Compound 102

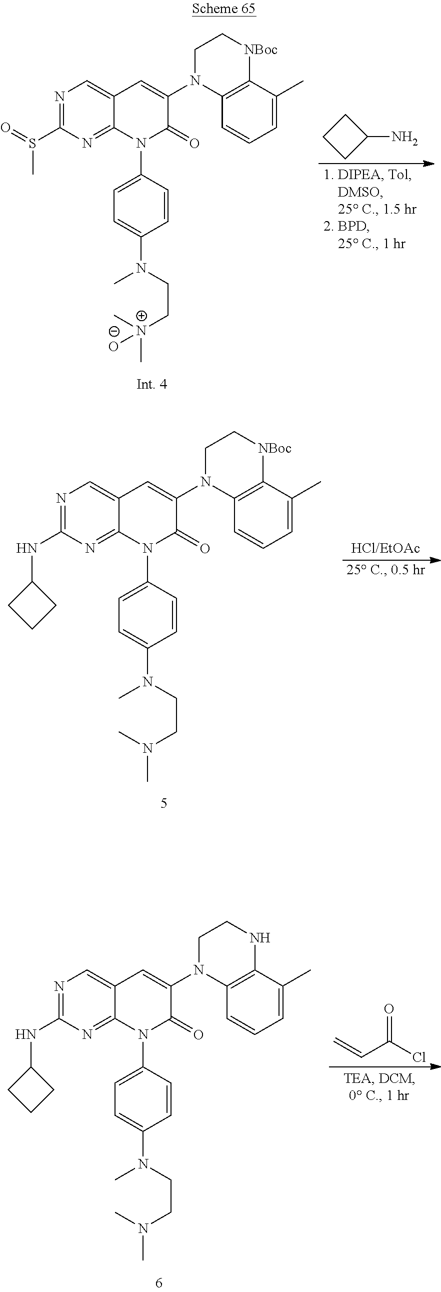

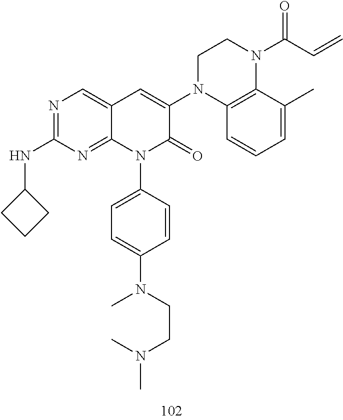 [SMILES: C=CC(=O)N1CCN(c2cc3cnc(NC4CCC4)nc3n(-c3ccc(N(C)CCN(C)C)cc3)c2=O)c2cccc(C)c21]

To a solution of 2-[4-[6-(4-tert-butoxycarbonyl-5-methyl-2,3-dihydroquinoxalin-1-yl)-2-methylsulfinyl-7-oxo-pyrido[2,3-d]pyrimidin-8-yl]—N-methyl-anilino]—N, N-dimethyl-ethanamine oxide (200.0 mg, 308.7 μmol, 1 eq) in DMSO (1 mL) and Tol. (2 mL) was added cyclobutanamine (26.3 mg, 370.4 μmol, 31.7 μL, 1·2 eq) and DIEA (199.5 mg, 1.5 mmol, 268.8 μL, 5 eq). The mixture was stirred at 25° C. for 1.5 hr. The mixture was added BPD (235·2 mg, 926·2 μmol, 3 eq) and stirred at 25° C. for 1 hr. LC-MS showed desired compound was detected. The reaction mixture was concentrated under reduced pressure to remove solvent. The residue was diluted with H₂O 10 mL and extracted with ethyl acetate (10 mL×3). The combined organic layers were washed with brine (10 mL×2), dried over Na₂SO₄, filtered and concentrated under reduced pressure to give a residue. The residue was purified by prep-TLC (SiO₂, Dichloromethane: Methanol=10:1) to give compound tert-butyl 4-[2-(cyclobutylamino)-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (100 mg, 154.9 μmol, 50·2% yield, 99% purity) as a yellow

A solution of tert-butyl 4-[2-(cyclobutylamino)-8-[4-[2-(dimethylamino)ethyl-methyl-amino]phenyl]-7-oxo-pyrido[2,3-d]pyrimidin-6-yl]-8-methyl-2,3-dihydroquinoxaline-1-carboxylate (85 mg, 133.0 μmol, 1 eq) in HCl/EtOAc (2 mL, 4M) was stirred at 25° C. for 0.5 hr. LC-MS showed desired compound was detected. The reaction mixture was concentrated under reduced pressure to give compound 2-(cyclobutylamino)-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (76.5 mg, crude, HCl) as a yellow solid.

To a solution of 2-(cyclobutylamino)-8-[4-[2-(dimethylamino)ethyl-methyl-amino]phenyl]-6-(5-methyl-3,4-dihydro-2H-quinoxalin-1-yl)pyrido[2,3-d]pyrimidin-7-one (76.5 mg, 118.3 μmol, 1 eq, HCl) in DCM (2 mL) was added TEA (35.9 mg, 355.1 μmol, 49.4 μL, 3 eq). The mixture was added dropwise prop-2-enoyl chloride (10.7 mg, 118.3 μmol, 9.6 μL, 1 eq) in DCM (0.1 mL) at 0° C. and stirred at 0° C. for 0.5 hr. LC-MS showed 2-(cyclobutylamino)-8-[4-[2-(dimethylamino)ethyl-methyl-amino]phenyl]-6-(5-methyl-3,4-dihydro-2H-quinoxalin-1-yl)pyrido[2,3-d]pyrimidin-7-one remained. The mixture was added dropwise prop-2-enoyl chloride (4·2 mg, 47.3 μmol, 3.8 μL, 0.4 eq) in DCM (0.1 mL) at 0° C. and stirred at 0° C. for 0.5 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min) to give 2-(cyclobutylamino)-8-[4-[2-(dimethylamino)ethyl-methyl-amino]phenyl]-6-(5-methyl-4-prop-2-enoyl-2,3-dihydroquinoxalin-1-yl)pyrido[2,3-d]pyrimidin-7-one (44.7 mg, 62.7 μmol, 52.9% yield, 99% purity, TFA) as a yellow

### Procedure for Preparation of Compound 096

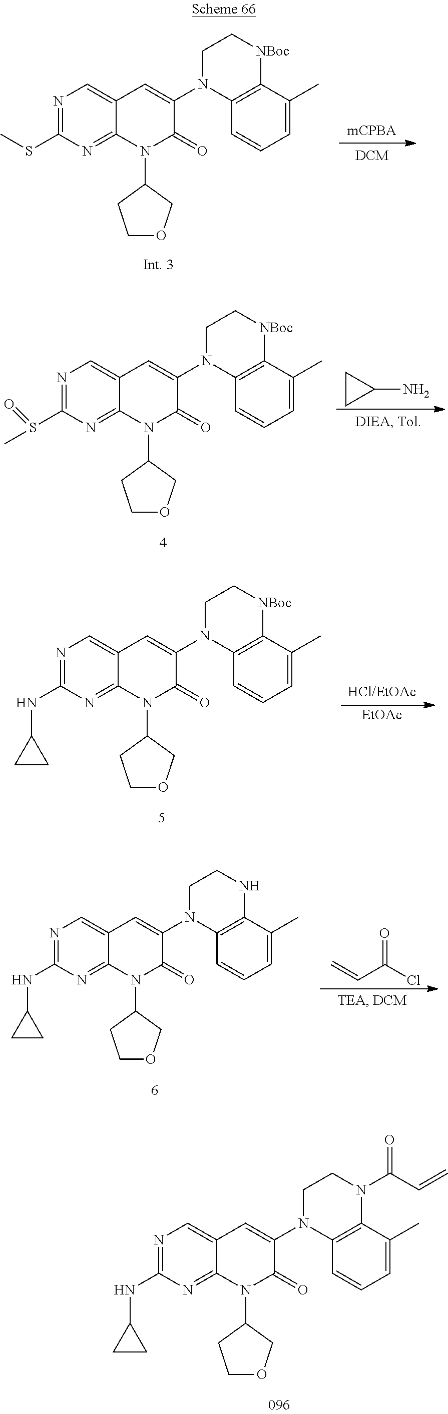

To a solution of tert-butyl 8-methyl-4-(2-methylsulfanyl-7-oxo-8-tetrahydrofuran-3-yl-pyrido[2,3-d]pyrimidin-6-yl)-2,3-dihydroquinoxaline-1-carboxylate (500 mg, 981.1 μmol, 1 eq) in DCM (10 mL) was added mCPBA (298.8 mg, 1.5 mmol, 85% purity, 1.5 eq) at 0° C. The mixture was stirred at 20° C. for 12 hr. LC-MS showed desired compound was detected. The residue was diluted with Na₂SO₃ (30 mL) and extracted with dichloromethane (30 mL× 3). The combined organic layers were washed with NaHCO₃ (15 mL× 2), dried over Na₂SO₄, filtered and concentrated under reduced pressure to give tert-butyl 8-methyl-4-(2-methylsulfinyl-7-oxo-8-tetrahydrofuran-3-yl-pyrido[2,3-d]pyrimidin-6-yl)-2,3-dihydroquinoxaline-1-carboxylate (520 mg, crude) as a yellow solid.

To a solution of tert-butyl 8-methyl-4-(2-methylsulfinyl-7-oxo-8-tetrahydrofuran-3-yl-pyrido[2,3-d]pyrimidin-6-yl)-2,3-dihydroquinoxaline-1-carboxylate (250 mg, 475.6 μmol, 1 eq) in Tol. (2.5 mL) was added DIEA (307.4 mg, 2.4 mmol, 414·2 μL, 5 eq) and cyclopropanamine (40.7 mg, 713.5 μmol, 49.4 L, 1.5 eq). The mixture was stirred at 20° C. for 12 hr under N₂ atmosphere. LC-MS showed desired compound was detected. The reaction mixture was concentrated under reduced pressure to remove solvent. The residue was purified by flash silica gel chromatography (12 g Silica Flash Column, Eluent of 0˜36% Ethyl acetate/Petroleum ether gradient @ 80 mL/min) to give tert-butyl 4-[2-(cyclopropylamino)-7-oxo-8-tetrahydrofuran-3-yl-pyrido[2,3-d]pyrimidin-6-yl]-8-methyl-2,3-dihydroquinoxaline-1-carboxylate (215 mg, 414.6 μmol, 87·2% yield) as a yellow solid.

A solution of tert-butyl 4-[2-(cyclopropylamino)-7-oxo-8-tetrahydrofuran-3-yl-pyrido[2,3-d]pyrimidin-6-yl]-8-methyl-2,3-dihydroquinoxaline-1-carboxylate (215 mg, 414.6 μmol, 1 eq) in HCl/EtOAc (2 mL, 4M) and EtOAc (2 mL) was stirred at 20° C. for 1 hr. LC-MS showed desired compound was detected. The reaction mixture was concentrated under reduced pressure to give 2-(cyclopropylamino)-6-(5-methyl-3,4-dihydro-2H-quinoxalin-1-yl)-8-tetrahydrofuran-3-ylpyrido[2,3-d]pyrimidin-7-one (188 mg, crude, HCl) as a yellow solid.

To a solution of 2-(cyclopropylamino)-6-(5-methyl-3,4-dihydro-2H-quinoxalin-1-yl)-8-tetrahydrofuran-3-yl-pyrido[2,3-d]pyrimidin-7-one (70 mg, 153.86 μmol, 1 eq, HCl) in DCM (1 mL) was added TEA (23.4 mg, 230.8 μmol, 32.1 μL, 1.5 eq) and prop-2-enoyl chloride (13.9 mg, 153.9 μmol, 12.5 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition column: column: Phenomenex luna C18 100×40 mm×3 μm; mobile phase: [H₂O (0.1% TFA)-ACN]; gradient: 15%-55% B over 8.0 min) to give 2-(cyclopropylamino)-6-(5-methyl-4-prop-2-enoyl-2,3-dihydroquinoxalin-1-yl)-8-tetrahydrofuran-3-yl-pyrido[2,3-d]pyrimidin-7-one (26.4 mg, 55.8 μmol, 36.3% yield, 100% purity) as a yellow solid

### Procedure for Preparation of Compound 106

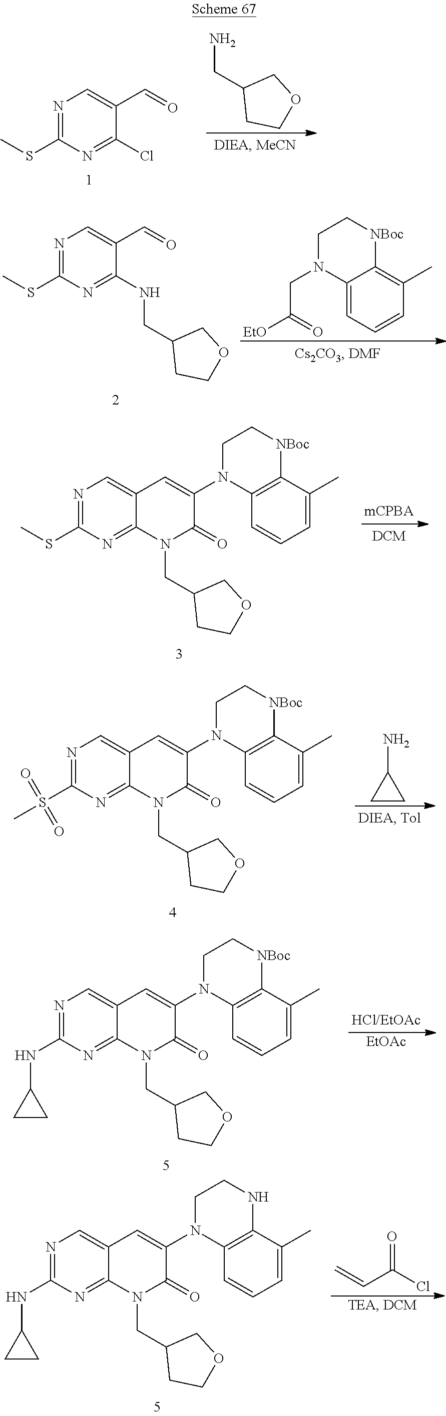

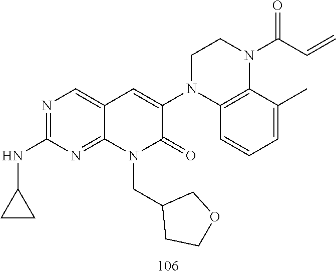 [SMILES: C=CC(=O)N1CCN(c2cc3cnc(NC4CC4)nc3n(CC3CCOC3)c2=O)c2cccc(C)c21]

To a solution of tetrahydrofuran-3-ylmethanamine (487.4 mg, 4.8 mmol, 1 eq) in MeCN (15 mL) was added DIEA (1.8 g, 14.4 mmol, 2.5 mL, 3 eq) and 4-chloro-2-methylsulfanyl-pyrimidine-5-carbaldehyde (1 g, 5.3 mmol, 1.1 eq). The mixture was stirred at 25° C. for 12 hr. LC-MS showed the desired compound was detected. The residue was diluted with H₂O (30 mL) and extracted with ethyl acetate (20 mL× 3). The combined organic layers were washed with brine (20 mL× 3), dried over Na₂SO₄, filtered and concentrated under reduced pressure to give a residue. The residue was purified by flash silica gel chromatography (20 g Silica Flash Column, Eluent of 0˜21% Ethyl acetate/Petroleum ether gradient @ 100 mL/min) to give 2-methylsulfanyl-4-(tetrahydrofuran-3-ylmethylamino)pyrimidine-5-carbaldehyde (900 mg, 3.5 mmol, 73.7% yield) as a white solid.

To a solution of 2-methylsulfanyl-4-(tetrahydrofuran-3-ylmethylamino)pyrimidine-5-carbaldehyde (900 mg, 3.5 mmol, 1 eq) in DMF (20 mL) was added Cs₂CO₃ (3.4 g, 10.6 mmol, 3 eq) and tert-butyl 4-(2-ethoxy-2-oxo-ethyl)-8-methyl-2,3-dihydroquinoxaline-1-carboxylate (1.1 g, 3.5 mmol, 1 eq). The mixture was stirred at 60° C. for 2 hr. LC-MS showed the desired compound was detected. The residue was diluted with H₂O 30 mL and extracted with ethyl acetate (20 mL× 3). The combined organic layers were washed with brine (20 mL× 3), dried over Na₂SO₄, filtered and concentrated under reduced pressure to give a residue. The residue was purified by flash silica gel chromatography (20 g Silica Flash Column, Eluent of 0˜18% Ethyl acetate/Petroleum ether gradient @ 100 mL/min) to give tert-butyl 8-methyl-4-[2-methylsulfanyl-7-oxo-8-(tetrahydrofuran-3-ylmethyl)pyrido[2,3-d]pyrimidin-6-yl]-2,3-dihydroquinoxaline-1-carboxylate (1.4 g, 2.4 mmol, 68.8% yield, 91.5% purity) as a white solid.

To a solution of tert-butyl 8-methyl-4-[2-methylsulfanyl-7-oxo-8-(tetrahydrofuran-3-ylmethyl)pyrido[2,3-d]pyrimidin-6-yl]-2,3-dihydroquinoxaline-1-carboxylate (700 mg, 1.3 mmol, 1 eq) in DCM (10 mL) was added mCPBA (407.0 mg, 2.0 mmol, 85% purity, 1.5 eq) at 0° C. The mixture was stirred at 25° C. for 2 hr. LC-MS showed the desired compound was detected. The reaction mixture was quenched by addition Na₂SO₃ (10 mL) at 25° C., and then diluted with H₂O (10 mL) and extracted with dichloromethane (10 mL× 3). The combined organic layers were washed with NaHCO₃ (5 mL×3), dried over Na₂SO₄, filtered and concentrated under reduced pressure to give tert-butyl 8-methyl-4-[2-methylsulfonyl-7-oxo-8-(tetrahydrofuran-3-ylmethyl)pyrido[2,3-d]pyrimidin-6-yl]-2,3-dihydroquinoxaline-1-carboxylate (800 mg, crude) as a yellow solid.

To a solution of tert-butyl 8-methyl-4-[2-methylsulfonyl-7-oxo-8-(tetrahydrofuran-3-ylmethyl)pyrido[2,3-d]pyrimidin-6-yl]-2,3-dihydroquinoxaline-1-carboxylate (200 mg, 359.9 μmol, 1 eq) in Tol. (2 mL) was added DIEA (232.6 mg, 1.8 mmol, 313.4 μL, 5 eq) and cyclopropanamine (30.8 mg, 539.9 μmol, 37.4 μL, 1.5 eq). The mixture was stirred at 25° C. for 12 hr. LC-MS showed the desired compound was detected. The reaction mixture was concentrated under reduced pressure to remove solvent. The residue was purified by prep-TLC (SiO₂, Petroleum ether: Ethyl acetate=0:1) to give tert-butyl 4-[2-(cyclopropylamino)-7-oxo-8-(tetrahydrofuran-3-ylmethyl)pyrido[2,3-d]pyrimidin-6-yl]-8-methyl-2,3-dihydroquinoxaline-1-carboxylate (136 mg, 244.1 μmol, 67.8% yield, 95.6% purity) as a yellow solid.

A mixture of tert-butyl 4-[2-(cyclopropylamino)-7-oxo-8-(tetrahydrofuran-3-ylmethyl)pyrido[2,3-d]pyrimidin-6-yl]-8-methyl-2,3-dihydroquinoxaline-1-carboxylate (136 mg, 255.3 μmol, 1 eq) in HCl/EtOAc (2 mL) 4M and EtOAc (2 mL) was stirred at 20° C. for 1 hr. LC-MS showed the desired compound was detected. The reaction mixture was concentrated under reduced pressure to give 2-(cyclopropylamino)-6-(5-methyl-3,4-dihydro-2H-quinoxalin-1-yl)-8-(tetrahydrofuran-3-ylmethyl)pyrido[2,3-d]pyrimidin-7-one (120 mg, crude, HCl) as a yellow solid.

To a solution of 2-(cyclopropylamino)-6-(5-methyl-3,4-dihydro-2H-quinoxalin-1-yl)-8-(tetrahydrofuran-3-ylmethyl)pyrido[2,3-d]pyrimidin-7-one (100 mg, 213·2 μmol, 1 eq, HCl) in DCM (5 mL) was added TEA (64.7 mg, 639.6 μmol, 89.0 μL, 3 eq). Then the mixture was added prop-2-enoyl chloride (19.3 mg, 213·2 μmol, 17.3 μL, 1 eq) at 0° C. The mixture was stirred at 0° C. for 15 min. LC-MS showed the desired compound was detected. The reaction mixture was concentrated under N₂ to remove solvent. The residue was purified by prep-HPLC (TFA condition: column: Phenomenex luna C18 100×40 mm×3 μm; mobile phase: [H₂O (0.1% TFA)-ACN]; gradient: 25%-55% B over 8.0 min) to give 2-(cyclopropylamino)-6-(5-methyl-4-prop-2-enoyl-2,3-dihydroquinoxalin-1-yl)-8-(tetrahydrofuran-3-ylmethyl)pyrido[2,3-d]pyrimidin-7-one (57.5 mg, 117.3 μmol, 55.0% yield, 99.1% purity) as a yellow solid.

### Procedure for Preparation of Compound 097

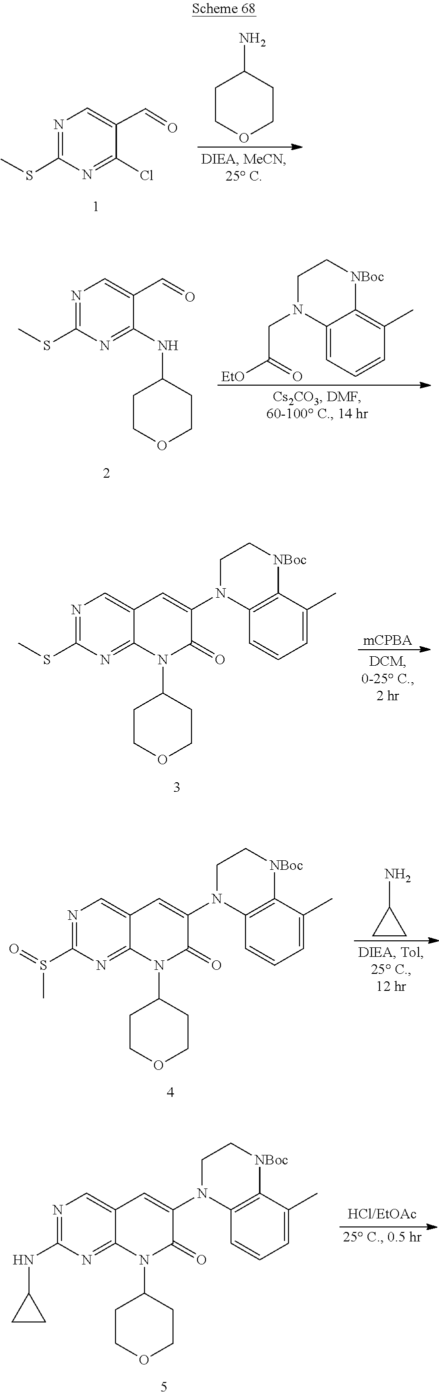

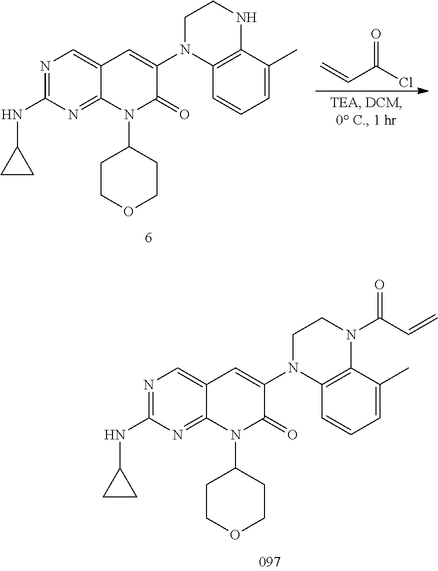

To a solution of tetrahydropyran-4-amine (487.4 mg, 4.8 mmol, 1 eq) in MeCN (15 mL) was added DIEA (1.8 g, 14.4 mmol, 2.5 mL, 3 eq) and 4-chloro-2-methylsulfanyl-pyrimidine-5-carbaldehyde (1 g, 5.3 mmol, 1.1 eq). The mixture was stirred at 25° C. for 12 hr. The mixture was added HCl (1 M, 4.8 mL, 1 eq) to adjust pH to 5-6. The mixture was stirred at 25° C. for 10 min. LC-MS showed desired compound was detected. The reaction mixture was concentrated under reduced pressure to remove solvent. The residue was purified by flash silica gel chromatography (12 g Silica Flash Column, Eluent of 0˜14% Ethyl acetate/Petroleum ether gradient @ 100 mL/min) to give 2-methylsulfanyl-4-(tetrahydropyran-4-ylamino)pyrimidine-5-carbaldehyde (1.1 g, 4.0 mmol, 41.9% yield, 93% purity) as a white solid.

To a solution of 2-methylsulfanyl-4-(tetrahydropyran-4-ylamino)pyrimidine-5-carbaldehyde (1.1 g, 4.3 mmol, 1 eq) in DMF (30 mL) was added Cs₂CO₃ (4·2 g, 13.0 mmol, 3 eq) and tert-butyl 4-(2-ethoxy-2-oxo-ethyl)-8-methyl-2,3-dihydroquinoxaline-1-carboxylate (1.4 g, 4.3 mmol, 1 eq). The mixture was stirred at 60° C. for 12 hr. The mixture was stirred at 100° C. for 2 hr. LC-MS showed desired compound was detected. The residue was diluted with H₂O (20 mL) and extracted with ethyl acetate (10 mL× 3). The combined organic layers were washed with brine (10 mL× 3), dried over Na₂SO₄, filtered, and concentrated under reduced pressure to give a residue. The residue was purified by flash silica gel chromatography (12 g Silica Flash Column, Eluent of 0˜33% Ethyl acetate/Petroleum ether gradient @ 100 mL/min) to give tert-butyl 8-methyl-4-(2-methylsulfanyl-7-oxo-8-tetrahydropyran-4-yl-pyrido[2,3-d]pyrimidin-6-yl)-2,3-dihydroquinoxaline-1-carboxylate (886 mg, 1·2 mmol, 29.6% yield, 76% purity) as a yellow solid.

To a solution of tert-butyl 8-methyl-4-(2-methylsulfanyl-7-oxo-8-tetrahydropyran-4-yl-pyrido[2,3-d]pyrimidin-6-yl)-2,3-dihydroquinoxaline-1-carboxylate (886 mg, 1.6 mmol, 1 eq) in DCM (15 mL) was added mCPBA (515·2 mg, 2.5 mmol, 85% purity, 1.5 eq) at 0° C. The mixture was stirred at 25° C. for 2 hr. LC-MS showed desired compound was detected. The reaction mixture was quenched by addition Na₂SO₃ (20 mL), extracted with dichloromethane (20 mL×3). The combined organic layers were washed with NaHCO₃ (20 mL× 2), and dried over Na₂SO₄, filtered and concentrated under reduced pressure to give tert-butyl 8-methyl-4-(2-methylsulfinyl-7-oxo-8-tetrahydropyran-4-yl-pyrido[2,3-d]pyrimidin-6-yl)-2,3-dihydroquinoxaline-1-carboxylate (1 g, crude) as a red solid.

To a solution of tert-butyl 8-methyl-4-(2-methylsulfinyl-7-oxo-8-tetrahydropyran-4-yl-pyrido[2,3-d]pyrimidin-6-yl)-2,3-dihydroquinoxaline-1-carboxylate (200 mg, 370.6 μmol, 1 eq) in Tol. (3 mL) was added DIEA (239.4 mg, 1.8 mmol, 322.7 μL, 5 eq) and cyclopropanamine (31.7 mg, 555.9 μmol, 38.5 μL, 1.5 eq). The mixture was stirred at 25° C. for 12 hr. LC-MS showed desired compound was detected. The reaction mixture was concentrated under reduced pressure to remove solvent. The residue was purified by flash silica gel chromatography (12 g Silica Flash Column, Eluent of 0˜20% Ethyl acetate/Petroleum ether gradient @ 100 mL/min) to give tert-butyl 4-[2-(cyclopropylamino)-7-oxo-8-tetrahydropyran-4-yl-pyrido[2,3-d]pyrimidin-6-yl]-8-methyl-2,3-dihydroquinoxaline-1-carboxylate (689 mg, 1·2 mmol, 64.9% yield, 93% purity) as a yellow solid.

To a vial containing tert-butyl 4-[2-(cyclopropylamino)-7-oxo-8-tetrahydropyran-4-yl-pyrido[2,3-d]pyrimidin-6-yl]-8-methyl-2,3-dihydroquinoxaline-1-carboxylate (308.1 mg, 578.4 μmol, 1 eq) was added HCl/EtOAc (4 M, 2.9 mL, 20.5 eq). The reaction was stirred at 25° C. for 0.5 hr. LC-MS showed desired compound was detected. The reaction mixture was concentrated under reduced pressure to give a yellow residue. The crude product 2-(cyclopropylamino)-6-(5-methyl-3,4-dihydro-2H-quinoxalin-1-yl)-8-tetrahydropyran-4-yl-pyrido[2,3-d]pyrimidin-7-one (304 mg, 541.9 μmol, 93.6% yield, 83.6% purity, HCl) was obtained as a yellow solid and was used into the next step without further purification.

To a solution of 2-(cyclopropylamino)-6-(5-methyl-3,4-dihydro-2H-quinoxalin-1-yl)-8-tetrahydropyran-4-yl-pyrido[2,3-d]pyrimidin-7-one (100 mg, 189.7 μmol, 1 eq, HCl) in DCM (2 mL) was added TEA (57.6 mg, 569.3 μmol, 79·2 μL, 3 eq). The mixture was added dropwise prop-2-enoyl chloride (17.1 mg, 189.7 μmol, 15.4 μL, 1 eq) in DCM (0.3 mL) at 0° C. and stirred at 0° C. for 0.5 hr. LC-MS showed 2-(cyclopropylamino)-6-(5-methyl-3,4-dihydro-2H-quinoxalin-1-yl)-8-tetrahydropyran-4-yl-pyrido[2,3-d]pyrimidin-7-one remained. The mixture was added dropwise prop-2-enoyl chloride (3.4 mg, 37.9 μmol, 3.0 μL, 0·2 eq) in DCM (0.1 mL) at 0° C. and stirred at 0° C. for 0.5 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-50% B over 8.0 min) to give 2-(cyclopropylamino)-6-(5-methyl-4-prop-2-enoyl-2,3-dihydroquinoxalin-1-yl)-8-tetrahydropyran-4-yl-pyrido[2,3-d]pyrimidin-7-one (53.8 mg, 110.6 μmol, 58.3% yield, 100% purity) as a yellow solid.

### Procedure for Preparation of Compound 105

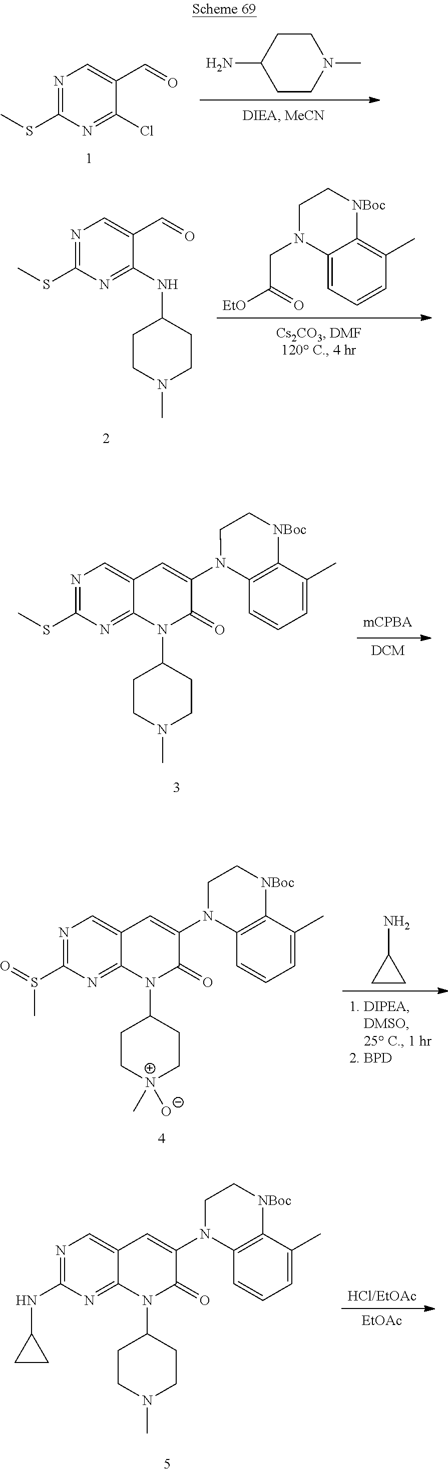

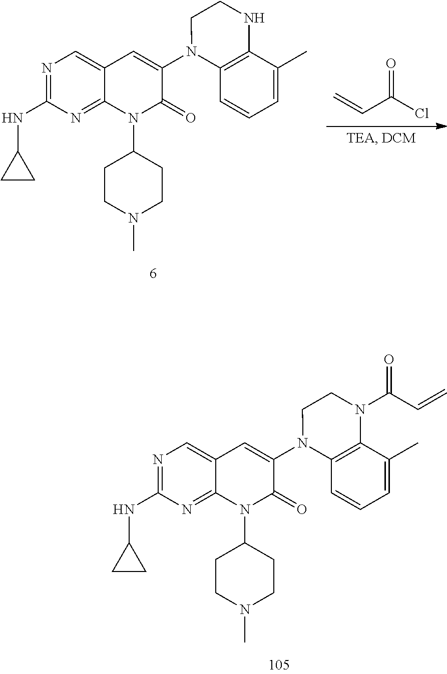

To a solution of 4-chloro-2-methylsulfanyl-pyrimidine-5-carbaldehyde (500 mg, 2.7 mmol, 1 eq) in MeCN (10 mL) was added DIEA (1.0 g, 8.0 mmol, 1.4 mL, 3 eq) and 1-methylpiperidin-4-amine (302.7 mg, 2.7 mmol, 1 eq). The mixture was stirred at 20° C. for 12 hr. LC-MS showed desired compound was detected. The reaction mixture was concentrated under reduced pressure to remove solvent. The residue was purified by flash silica gel chromatography (12 g Silica Flash Column, Eluent of 0˜12% Methanol/Dichloromethane ether gradient @ 80 mL/min) to give 4-[(1-methyl-4-piperidyl)amino]-2-methylsulfanyl-pyrimidine-5-carbaldehyde (550 mg, 2.1 mmol, 77.9% yield) as a yellow solid.

To a solution of 4-[(1-methyl-4-piperidyl)amino]-2-methylsulfanyl-pyrimidine-5-carbaldehyde (230 mg, 863.5 μmol, 1 eq) in DMF (6.5 mL) was added Cs₂CO₃ (844.0 mg, 2.6 mmol, 3 eq) and tert-butyl 4-(2-ethoxy-2-oxo-ethyl)-8-methyl-2,3-dihydroquinoxaline-1-carboxylate (288.8 mg, 863.5 μmol, 1 eq). The mixture was stirred at 120° C. for 4 hr. LC-MS showed desired compound was detected. The residue was diluted with H₂O (40 mL) and extracted with ethyl acetate (60 mL× 3). The combined organic layers were washed with aqueous NaCl (30 mL× 2), dried over Na₂SO₄, filtered and concentrated under reduced pressure to give a residue. The residue was purified by flash silica gel chromatography (12 g Silica Flash Column, Eluent of 0˜99% Ethyl acetate/Petroleum ether gradient @ 80 mL/min) to give tert-butyl 8-methyl-4-[8-(1-methyl-4-piperidyl)-2-methylsulfanyl-7-oxo-pyrido[2,3-d]pyrimidin-6-yl]-2,3-dihydroquinoxaline-1-carboxylate (250 mg) as a yellow solid.

To a solution of tert-butyl 8-methyl-4-[8-(1-methyl-4-piperidyl)-2-methylsulfanyl-7-oxo-pyrido[2,3-d]pyrimidin-6-yl]-2,3-dihydroquinoxaline-1-carboxylate (250 mg, 465.8 μmol, 1 eq) in DCM (3 mL) was added mCPBA (189.1 mg, 931.6 μmol, 85% purity, 2 eq) at 0° C. The mixture was stirred at 20° C. for 2 hr. LC-MS showed desired compound was detected. The residue was diluted with Na₂SO₃ (20 mL) and extracted with dichloromethane (20 mL×3). The combined organic layers were washed with NaHCO₃ (10 mL×2), dried over Na₂SO₄, filtered and concentrated under reduced pressure to give a residue to tert-butyl 8-methyl-4-[8-(1-methyl-1-oxido-piperidin-1-ium-4-yl)-2-methylsulfinyl-7-oxo-pyrido[2,3-d]pyrimidin-6-yl]-2,3-dihydroquinoxaline-1-carboxylate (250 mg, crude) as a yellow solid.

To a solution of tert-butyl 8-methyl-4-[8-(1-methyl-1-oxido-piperidin-1-ium-4-yl)-2-methylsulfinyl-7-oxo-pyrido[2,3-d]pyrimidin-6-yl]-2,3-dihydroquinoxaline-1-carboxylate (200 mg, 351.7 μmol, 1 eq) in DMSO (2 mL) was added DIPEA (227.3 mg, 1.8 mmol, 306.3 μL, 5 eq) and cyclopropanamine (30.1 mg, 527.5 μmol, 36.6 μL, 1.5 eq). The mixture was stirred at 20° C. for 1 hr. Then the mixture was added BPD (178.6 mg, 703.4 μmol, 2 eq). The mixture was stirred at 20° C. for 1 hr. LC-MS showed desired compound was detected. The reaction mixture was concentrated under reduced pressure to remove solvent. The residue was purified by prep-HPLC (FA condition column: Phenomenex Luna C18 100×30 mm×5 μm; mobile phase: [H₂O (0·2% FA)-ACN]; gradient: 15%-45% B over 8.0 min) to give tert-butyl 4-[2-(cyclopropylamino)-8-(1-methyl-4-piperidyl)-7-oxo-pyrido[2,3-d]pyrimidin-6-yl]-8-methyl-2,3-dihydroquinoxaline-1-carboxylate (115 mg, 183·26 μmol, 59.9% yield) as a yellow solid.

A mixture of tert-butyl 4-[2-(cyclopropylamino)-8-(1-methyl-4-piperidyl)-7-oxo-pyrido[2,3-d]pyrimidin-6-yl]-8-methyl-2,3-dihydroquinoxaline-1-carboxylate (115 mg, 210.8 μmol, 1 eq) in HCl/EtOAc (1 mL, 4M) and EtOAc (1 mL) was stirred at 20° C. for 40 min. LC-MS showed desired compound was detected. The reaction mixture was concentrated under reduced pressure to remove solvent to give 2-(cyclopropylamino)-6-(5-methyl-3,4-dihydro-2H-quinoxalin-1-yl)-8-(1-methyl-4-piperidyl)pyrido[2,3-d]pyrimidin-7-one (118 mg, crude, HCl) as a yellow solid.

To a solution of 2-(cyclopropylamino)-6-(5-methyl-3,4-dihydro-2H-quinoxalin-1-yl)-8-(1-methyl-4-piperidyl)pyrido[2,3-d]pyrimidin-7-one (100 mg, 207.5 μmol, 1 eq, HCl) in DCM (1 mL) was added TEA (63.0 mg, 622.4 μmol, 86.6 μL, 3 eq) and prop-2-enoyl chloride (18.8 mg, 207.5 μmol, 16.9 μL, 1 eq) at 0° C. The mixture was stirred at 0° C. for 0.5 hr. LC-MS showed desired compound was detected. The reaction mixture was concentrated under reduced pressure to remove solvent. The residue was purified by prep-HPLC (TFA condition column: Phenomenex luna C18 80×40 mm×3 μm; mobile phase: [H₂O (0.1% TFA)-ACN]; gradient: 10%-40% B over 8.0 min) to give 2-(cyclopropylamino)-8-(1-methyl-4-piperidyl)-6-(5-methyl-4-prop-2-enoyl-2,3-dihydroquinoxalin-1-yl)pyrido[2,3-d]pyrimidin-7-one (44.5 mg, 72.0 μmol, 34.7% yield, 99.3% purity, TFA) as a yellow solid.

### Procedure for Preparation of Compound 098

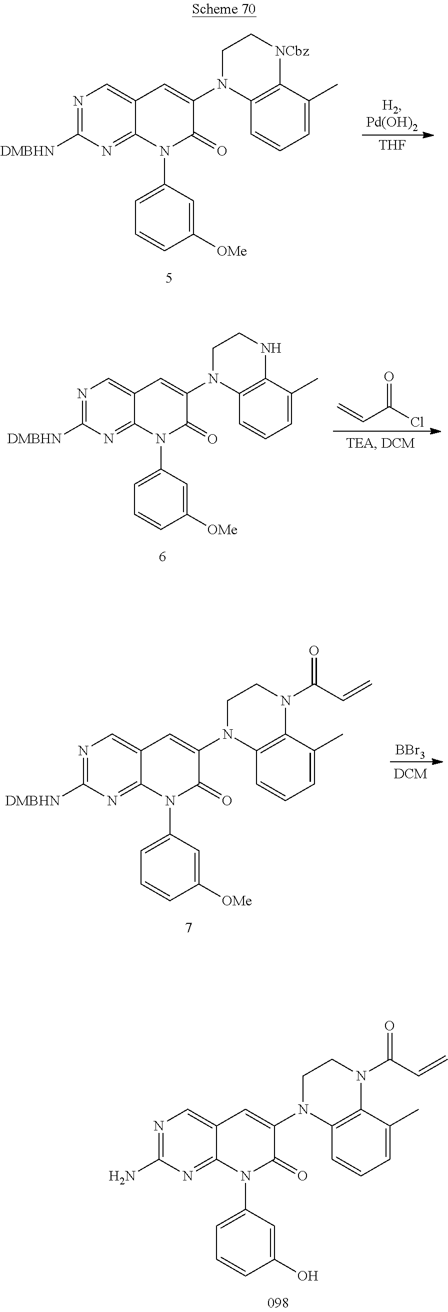

To a solution of Pd(OH)₂ (250 mg, 20% purity) in THF (10 mL) was added benzyl 4-[2-[(2,4-dimethoxyphenyl)methylamino]-8-(3-methoxyphenyl)-7-oxo-pyrido[2,3-d]pyrimidin-6-yl]-8-methyl-2,3-dihydroquinoxaline-1-carboxylate (500 mg, 715.6 μmol, 1 eq) and TEA (217·2 mg, 2·2 mmol, 298.8 μL, 3 eq) under N₂ atmosphere. The suspension was degassed and purged with H₂ for 3 times. The mixture was stirred under H₂ 15 Psi at 20° C. for 2 hr. LC-MS showed desired compound was detected. The reaction mixture was filtered, and the filtrate dried in vacuum to give Compound 2-[(2,4-dimethoxyphenyl)methylamino]-8-(3-methoxyphenyl)-6-(5-methyl-3,4-dihydro-2H-quinoxalin-1-yl)pyrido[2,3-d]pyrimidin-7-one (390 mg, crude) as a yellow

To a solution of 2-[(2,4-dimethoxyphenyl)methylamino]-8-(3-methoxyphenyl)-6-(5-methyl-3,4-dihydro-2H-quinoxalin-1-yl)pyrido[2,3-d]pyrimidin-7-one (200.0 mg, 354·2 μmol, 1 eq) in DCM (2 mL) was added TEA (107.5 mg, 1.1 mmol, 147.9 μL, 3 eq) and prop-2-enoyl chloride (32.1 mg, 354·2 μmol, 28.8 μL, 1 eq). The mixture was stirred at 0° C. for 2 hr. LC-MS showed 72% Reactant was remained and 25% of desired compound was detected. Then added prop-2-enoyl chloride (16.0 mg, 177.1 μmol, 14.4 μL, 0.5 eq) to the mixture. The mixture was stirred at 0° C. for 1 hr. LC-MS showed 44% Reactant was remained and 51% of desired compound was detected. Then added prop-2-enoyl chloride (16.0 mg, 177.1 μmol, 14.4 μL, 0.5 eq) to the mixture. The mixture was stirred at 0° C. for 1 hr. LC-MS showed 44% Reactant was remained and 51% of desired compound was detected. Then added prop-2-enoyl chloride (32.1 mg, 354·2 μmol, 28.8 μL, 1 eq) to the mixture. The mixture was stirred at 25° C. for 12 hr. LC-MS showed desired compound was detected. The reaction mixture was concentrated under reduced pressure to remove solvent. The residue was purified by prep-HPLC (TFA condition column: Phenomenex luna C18 100×40 mm×3 μm; mobile phase: [H₂O (0.1% TFA)-ACN]; gradient: 20%-50% B over 8.0 min) to give 2-[(2,4-dimethoxyphenyl)methylamino]-8-(3-methoxyphenyl)-6-(5-methyl-4-prop-2-enoyl-2,3-dihydroquinoxalin-1-yl)pyrido[2,3-d]pyrimidin-7-one (130 mg, 210.1 μmol, 59.3% yield) as a yellow solid.

To a solution of 2-[(2,4-dimethoxyphenyl)methylamino]-8-(3-methoxyphenyl)-6-(5-methyl-4-prop-2-enoyl-2,3-dihydroquinoxalin-1-yl)pyrido[2,3-d]pyrimidin-7-one (80.0 mg, 129.3 μmol, 1 eq) in DCM (1 mL) was added BBr₃ (162.0 mg, 646.5 μmol, 62.3 μL, 5 eq) at 0° C. The mixture was stirred at 20° C. for 2 hr. Then added BBr₃ (97·2 mg, 388.0 μmol, 37.4 μL, 3 eq) to the mixture at 0° C. Then the mixture was stirred at 20° C. for 36 hr. LC-MS showed desired compound was detected. The reaction mixture was quenched by addition ice water 10 mL at 0° C., and adjusted pH=8 with NH₃. H₂O. Then the mixture was extracted with DCM: i-PrOH=3:1 (10 mL× 3). The organic phase was separated, dried over Na₂SO₄, filtered and concentrated under reduced pressure to give a residue. DCM (2 mL) was added to the residue and then TEA (0.5 mL) was added. The mixture was stirred at 20° C. for 12 hr. Then the mixture was concentrated to get a residue. The residue was purified by prep-HPLC (TFA condition column: Phenomenex luna C18 100×40 mm×3 μm; mobile phase: [H₂O (0.1% TFA)-ACN]; gradient: 30%-60% B over 8.0 min) to give 2-amino-8-(3-hydroxyphenyl)-6-(5-methyl-4-prop-2-enoyl-2,3-dihydroquinoxalin-1-yl)pyrido[2,3-d]pyrimidin-7-one (17.0 mg, 37.4 μmol, 28.9% yield, 99.9% purity) as a yellow solid.

### Procedure for Preparation of Compound 100

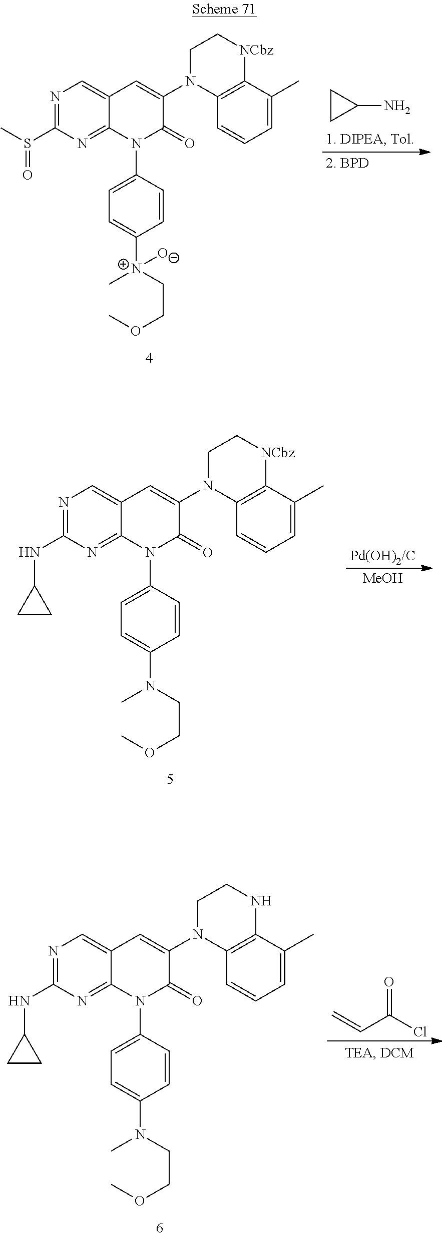

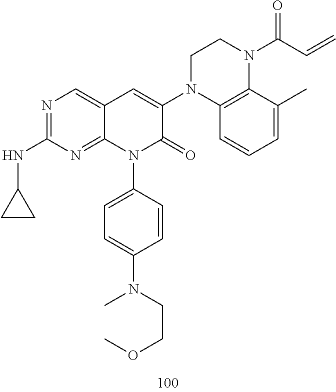 [SMILES: C=CC(=O)N1CCN(c2cc3cnc(NC4CC4)nc3n(-c3ccc(N(C)CCOC)cc3)c2=O)c2cccc(C)c21]

To a solution of 4-[6-(4-benzyloxycarbonyl-5-methyl-2,3-dihydroquinoxalin-1-yl)-2-methylsulfinyl-7-oxo-pyrido[2,3-d]pyrimidin-8-yl]—N-(2-methoxyethyl)-N-methyl-benzeneamine oxide (280 mg, 418.7 μmol, 1 eq) in Tol. (3 mL) was added DIPEA (162.3 mg, 1.3 mmol, 218.8 μL, 3 eq) and cyclopropanamine (35.9 mg, 628.0 μmol, 43.5 μL, 1.5 eq). The mixture was stirred at 20° C. for 1 hr. Then BPD (319.0 mg, 1.3 mmol, 3 eq) was added to the mixture. The mixture was stirred at 20° C. for 1 hr. LC-MS showed desired compound was detected. The residue was diluted with H₂O 20 mL and extracted with ethyl acetate (20 mL×3). The combined organic layers were washed with brine (10 mL× 2), dried over Na₂SO₄, filtered and concentrated under reduced pressure to give a residue. The residue was purified by flash silica gel chromatography (4 g Silica Flash Column, Eluent of 0˜45% Ethyl acetate/Petroleum ether gradient @ 60 mL/min) to give benzyl 4-[2-(cyclopropylamino)-8-[4-[2-methoxyethyl(methyl)amino]phenyl]-7-oxo-pyrido[2,3-d]pyrimidin-6-yl]-8-methyl-2,3-dihydroquinoxaline-1-carboxylate (210 mg, 325·2 μmol, 77.7% yield) as a yellow solid.

To a solution of Pd(OH)₂/C (100 mg, 20% purity) in MeOH (10 mL) was added benzyl 4-[2-(cyclopropylamino)-8-[4-[2-methoxyethyl(methyl)amino]phenyl]-7-oxo-pyrido[2,3-d]pyrimidin-6-yl]-8-methyl-2,3-dihydroquinoxaline-1-carboxylate (160 mg, 247.8 μmol, 1 eq) under N₂ atmosphere. The suspension was degassed and purged with H₂ for 3 times. The mixture was stirred under H₂ (15 psi) at 20° C. for 1 hr. LC-MS showed desired compound was detected. The reaction mixture was filtered and concentrated under reduced pressure to give 2-(cyclopropylamino)-8-[4-[2-methoxyethyl(methyl)amino]phenyl]-6-(5-methyl-3,4-dihydro-2H-quinoxalin-1-yl)pyrido[2,3-d]pyrimidin-7-one (130 mg, crude) as a yellow solid.

To a solution of 2-(cyclopropylamino)-8-[4-[2-methoxyethyl(methyl)amino]phenyl]-6-(5-methyl-3,4-dihydro-2H-quinoxalin-1-yl)pyrido[2,3-d]pyrimidin-7-one (130 mg, 254.1 μmol, 1 eq) in DCM (2 mL) was added TEA (77.1 mg, 762.3 μmol, 106.1 μL, 3 eq) and prop-2-enoyl chloride (23.0 mg, 254.1 μmol, 20.6 μL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition; column: Phenomenex luna C18 100×40 mm×3 μm; mobile phase: [H₂O (0.1% TFA)-ACN]; gradient: 20%-55% B over 8.0 min) to give 2-(cyclopropylamino)-8-[4-[2-methoxyethyl(methyl)amino]phenyl]-6-(5-methyl-4-prop-2-enoyl-2,3-dihydroquinoxalin-1-yl)pyrido[2,3-d]pyrimidin-7-one (62.3 mg, 107.3 μmol, 42·2% yield, 97.5% purity) as a yellow

Characterization data for Compounds 001-106 are presented below in Table 3.

TABLE 3

| Compound | LCMS | |
|---|---|---|
| No. | (M + H) | ¹H NMR (DMSO unless stated) |
| 001 | 510.2 | δ: 10.03-9.93 (m, 1H), 9.88-9.74 (m, 1H), 8.67-8.60 (m, |
| | | 1H), 8.25 (s, 1H), 7.70-7.56 (m, 4H), 7.31 (dd, J = 1.3, 7.6 |
| | | Hz, 1H), 7.23 (dt, J = 1.3, 7.7 Hz, 1H), 7.16-7.07 (m, 1H), |
| | | 6.92 (d, J = 9.1 Hz, 2H), 6.59-6.44 (m, 1H), 6.31-6.19 (m, |
| | | 1H), 5.81-5.71 (m, 1H), 3.83 (s, 2H), 3.60 (s, 3H), 3.11- |
| | | 3.04 (m, 4H), 2.47-2.41 (m, 4H), 2.21 (s, 3H) |
| 002 | 512.2 | δ: 9.93-9.83 (m, 1H), 9.75 (s, 1H), 8.74-8.69 (m, 1H), 8.17 |
| | | (br d, J = 7.9 Hz, 1H), 7.65 (br d, J = 8.8 Hz, 2H), 7.62-7.59 |
| | | (m, 1H), 7.16-7.04 (m, 2H), 7.02-6.90 (m, 3H), 6.71 (dd, J = |
| | | 10.2, 16.9 Hz, 1H), 6.31-6.22 (m, 1H), 5.78-5.71 (m, |
| | | 1H), 3.64 (s, 3H), 3.14-3.05 (m, 4H), 2.48-2.44 (m, 4H), |
| | | 2.23 (s, 3H) |
| 003 | 538.2 | δ: 9.80 (br s, 1H), 8.71-8.65 (m, 1H), 7.67-7.60 (m, 2H), |
| | | 7.47 (s, 1H), 7.10-7.01 (m, 2H), 6.93 (d, J = 9.0 Hz, 2H), |
| | | 6.85-6.75 (m, 2H), 6.14-6.06 (m, 1H), 5.64-5.57 (m, 1H), |
| | | 4.19-4.13 (m, 2H), 3.64-3.60 (m, 3H), 3.10-3.04 (m, 6H), |
| | | 2.47-2.43 (m, 4H), 2.22 (s, 3H) |
| 004 | 542.2 | δ: 9.80-9.64 (m, 1H), 8.71 (br d, J = 5.3 Hz, 1H), 8.49 (s, |
| | | 1H), 8.20 (br d, J = 5.9 Hz, 1H), 7.75-7.63 (m, 1H), 7.59- |
| | | 7.57 (m, 1H), 7.13-7.02 (m, 2H), 7.00-6.95 (m, 1H), 6.75- |
| | | 6.66 (m, 1H), 6.66-6.63 (m, 1H), 6.55-6.50 (m, 1H), 6.25 |
| | | (dd, J = 1.9, 17.1 Hz, 1H), 5.76-5.70 (m, 1H), 3.81 (s, 3H), |
| | | 3.56 (s, 3H), 3.17-3.13 (m, 4H), 2.45 (br s, 4H), 2.23 (s, 3H) |
| 005 | 513.1 | δ: 9.84 (br s, 1H), 9.75-9.71 (m, 1H), 8.71 (s, 1H), 8.52 (br |
| | | s, 1H), 8.27 (br s, 1H), 8.16 (br d, J = 7.4 Hz, 1H), 7.91 (br |
| | | d, J = 8.0 Hz, 1H), 7.59 (s, 1H), 7.13-7.03 (m, 2H), 7.00- |
| | | 6.95 (m, 1H), 6.86 (d, J = 9.1 Hz, 1H), 6.71 (dd, J = 10.2, |
| | | 16.9 Hz, 1H), 6.29-6.22 (m, 1H), 5.76-5.71 (m, 1H), 3.61 |
| | | (s, 3H), 3.46-3.41 (m, 4H), 2.43-2.39 (m, 4H), 2.22 (s, 3H) |
| 006 | 510.3 | N/A |
| 007 | 623.5 | δ: 9.96-9.77 (m, 1H), 9.71 (br s, 1H), 8.71 (br s, 1H), 8.24- |
| | | 8.21 (m, 1H), 8.16 (br d, J = 5.6 Hz, 1H), 7.63-7.55 (m, |
| | | 1H), 7.54-7.42 (m, 2H), 7.14-7.02 (m, 2H), 6.98 (br d, J = |
| | | 7.5 Hz, 1H), 6.92-6.82 (m, 2H), 6.79-6.65 (m, 1H), 6.25 |
| | | (dd, J = 1.2, 16.9 Hz, 1H), 5.76-5.69 (m, 1H), 5.54-5.27 |
| | | (m, 1H), 4.53-4.33 (m, 1H), 4.17-3.71 (m, 3H), 3.12-3.06 |
| | | (m, 4H), 2.72-2.60 (m, 1H), 2.48-2.44 (m, 4H), 2.23 (s, |
| | | 3H), 2.17-1.96 (m, 2H), 1.91-1.67 (m, 3H), 1.56-1.30 (m, 1H) |
| 008 | 631.3 | δ: 10.24-10.14 (m, 1H), 9.88-9.75 (m, 1H), 9.72 (s, 1H), |
| | | 8.75 (s, 1H), 8.28-8.23 (m, 1H), 8.20-8.10 (m, 1H), 7.74 |
| | | (br d, J = 8.0 Hz, 1H), 7.70-7.62 (m, 2H), 7.52 (t, J = 8.0 |
| | | Hz, 1H), 7.20 (br d, J = 7.6 Hz, 2H), 7.15-7.07 (m, 3H), |
| | | 7.03 (br d, J = 8.0 Hz, 1H), 6.72 (dd, J = 10.1, 16.9 Hz, 1H), |
| | | 6.58 (br d, J = 6.1 Hz, 2H), 6.29-6.19 (m, 1H), 5.76-5.68 |
| | | (m, 1H), 2.99 (br s, 4H), 2.44 (br s, 4H), 2.22 (s, 3H), 2.04 |
| | | (s, 3H) |
| 009 | 489.2 | δ: 9.63 (s, 1H), 8.67-8.60 (m, 1H), 7.66-7.55 (m, 2H), 7.14- |
| | | 7.08 (m, 1H), 6.94-6.79 (m, 2H), 6.80-6.79 (m, 1H), 6.20- |
| | | 6.09 (m, 1H), 5.75-5.67 (m, 1H), 3.70 (br d, J = 8.4 Hz, |
| | | 4H), 3.61 (s, 3H), 3.11-3.01 (m, 8H), 2.47-2.42 (m, 4H), |
| | | 2.21 (s, 3H) |
| 010 | 537.2 | δ: 9.82-10.04 (m, 1 H) 8.65-8.79 (m, 1 H) 7.83-8.02 (m, |
| | | 1 H) 7.56-7.76 (m, 2 H) 6.84-7.20 (m, 4 H) 6.57-6.81 (m, |
| | | 2 H) 6.41-6.55 (m, 1 H) 6.16-6.39 (m, 1 H) 5.52 (d, J = 5.13 |
| | | Hz, 1 H) 3.93-4.07 (m, 2 H) 3.62 (s, 3 H) 3.56-3.61 (m, 2 |
| | | H) 3.16 (br s, 4 H) 2.63 (br s, 3 H) 2.35-2.41 (m, 4 H) |
| 011 | 565.2 | δ: 9.66 (s, 1H), 8.43 (s, 1H), 8.18-8.14 (m, 1H), 7.58 (d, J = |
| | | 9.0 Hz, 2H), 7.39-7.30 (m, 2H), 7.25 (t, J = 7.5 Hz, 2H), |
| | | 7.17 (d, J = 7.5 Hz, 1H), 7.11 (s, 1H), 6.93-6.80 (m, 3H), |
| | | 6.22-6.10 (m, 1H), 5.71 (br dd, J = 10.5, 17.5 Hz, 1H), 4.67- |
| | | 4.53 (m, 1H), 4.23-4.08 (m, 1H), 4.05-3.93 (m, 1H), 3.69- |
| | | 3.49 (m, 5H), 3.48-3.37 (m, 1H), 3.11-3.01 (m, 4H), 2.83- |
| | | 2.72 (m, 1H), 2.47-2.41 (m, 4H), 2.21 (s, 3H) |
| 012 | 515.2 | δ: 9.58 (s, 1 H), 8.69 (s, 1 H), 8.19 (s, 1 H), 7.63 (d, J = 9.01 |
| | | Hz, 2 H), 7.38 (br d, J = 17.26 Hz, 1 H), 6.91 (d, J = 9.13 Hz, 2 |
| | | H), 6.81 (ddd, J = 16.13, 10.57, 5.07 Hz, 1 H), 6.15 (br d, |
| | | J = 16.38 Hz, 1 H),5.69 (dd, J = 10.44, 2.06 Hz, 1 H), 3.69 (br |
| | | d, J = 16.26 Hz, 4 H), 3.60 (s, 3 H), 3.47 (br s, 2 H), 3.02- |
| | | 3.13 (m, 4 H),2.40-2.49 (m, 4 H), 2.23 (s, 3 H), 0.90 (br d, |
| | | J = 3.88 Hz, 2 H), 0.65 (br s, 2 H) |
| 013 | 491.0 | δ: 10.13 (br s, 1H), 8.72 (br s, 1H), 8.02 (br s, 1H), 7.95 (s, |
| | | 1H), 7.68-7.51 (m, 1H), 7.19 (br d, J = 7.0 Hz, 1H), 6.97- |
| | | 6.85 (m, 1H), 6.66 (br t, J = 7.3 Hz, 1H), 6.50 (br d, J = 8.0 |
| | | Hz, 1H), 5.47-5.20 (m, 2H), 4.91 (br s, 1H), 4.15 (br s, 2H), |
| | | 3.97 (br d, J = 5.0 Hz, 2H), 3.77-3.70 (m, 2H), 3.69-3.57 |
| | | (m, 5H) |
| 014 | 490.0 | δ: 10.04 (br s, 1H), 8.59 (s, 1H), 8.13-7.87 (m, 1H), 7.64 (s, |
| | | 1H), 7.42-7.31 (m, 2H), 7.26-7.18 (m, 1H), 7.15-7.08 (m, |
| | | 1H), 7.07-7.00 (m, 1H), 5.47-5.33 (m, 1H), 5.31 (s, 1H), |
| | | 4.92 (br s, 1H), 4.33 (br t, J = 6.6 Hz, 1H), 4.14 (br s, 2H), |
| | | 3.88-3.79 (m, 1H), 3.78-3.69 (m, 3H), 3.68-3.54 (m, 3H), |
| | | 2.26-2.10 (m, 2H). |
| 015 | 439.2 | δ: 9.75 (br s, 1 H), 8.62 (s, 1 H), 8.07 (d, J = 7.75 Hz, 1 H), |
| | | 7.98 (s, 1 H), 7.58 (brs, 1 H), 7.11 (s, 1 H), 6.21-6.31 (m, 1 |
| | | H), 6.06-6.15 (m, 1 H), 5.54-5.65 (m, 1 H), 4.89 (br s, 1 |
| | | H), 4.12 (br t, J = 5.50 Hz, 2 H), 3.86-3.97 (m, 1 H), 3.72 (br |
| | | t, J = 5.07 Hz, 2 H), 3.64 (br s, 3 H), 3.46 (br d, J = 11.26 Hz, 2 |
| | | H), 3.32 (br s, 1 H), 2.53-2.61 (m, 1 H), 1.76-1.89 (m, 2 |
| | | H), 1.57-1.70 (m, 1 H), 1.35-1.48 (m, 1 H) |
| 016 | 457.2 | δ: 9.65-9.88 (m, 1 H), 8.62 (s, 1 H), 8.36 (br d, J = 7.38 Hz, |
| | | 1 H), 7.98 (s, 1 H), 7.50-7.67 (m, 1 H), 7.13 (s, 1 H), 5.46- |
| | | 5.65 (m, 1 H), 5.28 (dd, J = 15.70, 3.31 Hz, 1 H), 4.71-5.04 |
| | | (m, 1 H), 4.12 (br t, J = 5.32 Hz, 2 H), 3.91-3.99 (m, 1 H), |
| | | 3.72 (br t, J = 5.50 Hz, 2 H), 3.64 (br s, 3 H), 3.44 (br d, |
| | | J = 11.38 Hz, 2 H), 2.67 (br t, J = 10.01 Hz, 1 H), 2.57 (br d, |
| | | J = 9.13 Hz, 1 H), 1.73-1.84 (m, 2 H), 1.55-1.69 (m, 2 H) |
| 017 | 472.1 | δ: 10.13-9.90 (m, 1H), 8.59 (br s, 1H), 7.99 (br s, 1H), 7.64 |
| | | (br s, 1H), 7.35-7.03 (m, 5H), 6.68 (dd, J = 10.3, 16.8 Hz, |
| | | 1H), 6.24 (dd, J = 2.2, 16.7 Hz, 1H), 5.78-5.70 (m, 1H), |
| | | 4.90 (br s, 1H), 4.28 (t, J = 6.4 Hz, 1H), 4.14 (br s, 2H), 3.84 |
| | | (td, J = 6.2, 12.8 Hz, 1H), 3.78-3.67 (m, 3H), 3.67-3.56 |
| | | (m, 3H), 2.22-2.04 (m, 2H) |
| 018 | 457.0 | δ: 9.95-9.62 (m, 1H), 8.63 (br s, 1H), 8.40 (s, 1H), 8.00 (s, |
| | | 1H), 7.71-7.55 (m, 1H), 7.11 (s, 1H), 5.79-5.57 (m, 2H), |
| | | 4.53 (br d, J = 4.0 Hz, 2H), 4.45 (br s, 2H), 3.63 (br s, 3H), |
| | | 3.60-3.57 (m, 2H), 3.05 (br d, J = 1.5 Hz, 1H), 2.61-2.53 |
| | | (m, 2H), 1.93 (br d, J = 7.6 Hz, 2H), 1.67-1.53 (m, 2H) |
| 019 | 517.4 | δ: 9.71-9.51 (m, 1H), 9.47-9.22 (m, 1H), 8.02 (s, 1H), 7.61 |
| | | (d, J = 9.0 Hz, 2H), 7.01-6.89 (m, 2H), 5.94-5.87 (m, 1H), |
| | | 5.29-5.25 (m, 1H), 4.76-4.63 (m, 1H), 4.52-4.37 (m, 1H), |
| | | 4.25 (s, 2H), 3.74 (br d, J = 13.7 Hz, 2H), 3.27 (s, 3H), 3.22- |
| | | 3.12 (m, 2H), 2.97-2.81 (m, 5H), 2.74-2.53 (m, 2H), 2.50- |
| | | 2.31 (m, 4H), 2.10-1.98 (m, 1H), 1.79-1.45 (m, 5H) |
| 020 | 503.1 | δ: 10.38 (br s, 1 H), 8.72 (s, 1 H), 7.95 (s, 1 H), 7.65 (d, |
| | | J = 2.13 Hz, 1 H), 6.90 (t, J = 8.32 Hz, 1 H), 6.70 (d, J = 1.25 |
| | | Hz, 1 H), 6.45 (d, J = 7.63 Hz, 1 H), 6.25-6.38 (m, 1 H), 6.19 |
| | | (d, J = 2.50 Hz, 1 H), 6.12-6.16 (m, 1 H), 5.58-5.66 (m, 1 |
| | | H), 4.89 (t, J = 5.32 Hz, 1 H), 4.74-4.86 (m, 1 H), 4.07 (t, |
| | | J = 5.63 Hz, 2 H), 3.71-3.75 (m, 5 H), 3.66 (br d, J = 9.51 Hz, |
| | | 1 H), 3.61 (s, 3 H), 3.35-3.46 (m, 1 H), 2.88-3.04 (m, 1 H) |
| 021 | 567.4 | δ: 2.25 (s, 3 H) 2.46-2.50 (m, 4 H) 2.89-3.03 (m, 1 H) 3.08- |
| | | 3.14 (m, 4 H) 3.44 (br s, 1 H) 3.62 (s, 3 H) 3.65-3.71 (m, |
| | | 1 H) 3.74 (s, 3 H) 4.84 (br dd, J = 5.38, 3.42 Hz, 1 H) 5.59- |
| | | 5.70 (m, 1 H) 6.12-6.24 (m, 2 H) 6.27-6.39 (m, 1 H) 6.46 |
| | | (d, J = 8.19 Hz, 1 H) 6.87-7.01 (m, 3 H) 7.68 (br d, J = 8.56 |
| | | Hz, 2 H) 7.95 (s, 1 H) 8.19 (s, 1 H) 8.73 (s, 1 H) 9.96 (br s, 1 H) |
| 022 | 517.4 | δ: 9.84 (br s, 1H), 8.68 (s, 1H), 8.25 (s, 1H), 7.64 (br d, J = |
| | | 8.7 Hz, 2H), 7.56 (s, 1H), 6.97-6.85 (m, 3H), 6.15 (dd, J = |
| | | 2.4, 16.7 Hz, 1H), 5.74-5.67 (m, 1H), 4.38 (br d, J = 11.9 |
| | | Hz, 1H), 4.05 (br d, J = 13.0 Hz, 1H), 3.59-3.54 (m, 3H), |
| | | 3.45-3.39 (m, 2H), 3.11-3.05 (m, 4H), 2.82 (br t, J = 11.9 |
| | | Hz, 1H), 2.48-2.39 (m, 5H), 2.22 (s, 3H), 0.78 (br d, J = 6.1 |
| | | Hz, 6H) |
| 023 | 529.3 | δ: 9.59 (br s, 1H), 8.62 (s, 1H), 8.26 (br s, 1H), 7.62 (d, J = |
| | | 8.9 Hz, 2H), 7.12 (d, J = 1.6 Hz, 1H), 6.91 (d, J = 9.0 Hz, |
| | | 2H), 6.64-6.51 (m, 1H), 6.13 (dd, J = 2.1, 16.8 Hz, 1H), |
| | | 5.70-5.62 (m, 1H), 3.70-3.59 (m, 6H), 3.27-3.11 (m, 5H), |
| | | 3.11-3.02 (m, 5H), 3.00-2.76 (m, 3H), 2.47-2.43 (m, 4H), |
| | | 2.22 (s, 3H), 1.90-1.72 (m, 1H), 1.64-1.47 (m, 1H) |
| 024 | 501.4 | δ: 9.51-9.64 (m, 1 H) 8.61-8.71 (m, 1 H) 7.58-7.68 (m, 2 |
| | | H) 7.27 (s, 1 H) 6.89-7.05 (m, 3 H) 6.17-6.30 (m, 1 H) |
| | | 5.73-5.85 (m, 1 H) 3.95-4.15 (m, 1 H) 3.61-3.67 (m, 3 H) |
| | | 3.51-3.57 (m, 1 H) 3.13-3.24 (m, 4 H) 3.06-3.10 (m, 4 H) |
| | | 2.41-2.47 (m, 4 H) 2.22 (s, 3 H) 1.05-1.13 (m, 1 H) 0.62- |
| | | 0.70 (m, 1 H) |
| 025 | 503.4 | δ: 10.06 (br s, 1H), 9.65 (br d, J = 3.9 Hz, 1H), 8.76 (s, 1H), |
| | | 7.91 (s, 1H), 7.71 (br d, J = 8.7 Hz, 2H), 7.02 (d, J = 9.0 Hz, |
| | | 2H), 6.94-6.76 (m, 1H), 6.21 (dd, J = 2.0, 16.7 Hz, 1H), |
| | | 5.77 (br d, J = 10.4 Hz, 1H), 4.41 (br s, 1H), 4.26 (s, 1H), |
| | | 3.98 (br d, J = 4.5 Hz, 2H), 3.88 (br s, 1H), 3.79 (br d, J = |
| | | 13.1 Hz, 2H), 3.61 (s, 3H), 3.52 (br d, J = 12.3 Hz, 2H), 3.23- |
| | | 3.12 (m, 2H), 2.99-2.84 (m, 5H) |
| 026 | 553.5 | δ: 9.66 (s, 1H), 8.70 (s, 1H), 7.82 (s, 1H), 7.75-7.66 (m, |
| | | 2H), 7.09-6.97 (m, 2H), 6.92-6.81 (m, 1H), 6.70-6.58 (m, |
| | | 1H), 6.41-6.12 (m, 1H), 3.75-3.56 (m, 7H), 3.35 (br s, 8H), |
| | | 2.88 (s, 3H), 2.45-1.89 (m, 5H), 1.16-0.96 (m, 3H) |
| 027 | 552.4 | δ: 9.57 (s, 1H), 8.63 (br d, J = 5.2 Hz, 1H), 7.84 (s, 1H), 7.69- |
| | | 7.58 (m, 2H), 7.00-6.85 (m, 3H), 6.69-6.61 (m, 1H), 6.45- |
| | | 6.21 (m, 3H), 5.82-5.65 (m, 1H), 5.08-4.66 (m, 1H), 4.52- |
| | | 4.31 (m, 1H), 3.75-3.58 (m, 5H), 3.56-3.41 (m, 3H), 2.90- |
| | | 2.81 (m, 2H), 2.56 (br s, 2H), 2.14 (s, 3H), 1.85 (br dd, J = |
| | | 3.4, 12.6 Hz, 2H), 1.64-1.44 (m, 2H) |
| 028 | 569.5 | δ: 9.87-10.10 (m, 1 H) 9.46-9.74 (m, 1 H) 8.65-8.79 (m, |
| | | 1 H) 7.91 (br s, 1 H) 7.63-7.78 (m, 2 H) 6.99-7.11 (m, 2 H) |
| | | 6.81-6.94 (m, 1 H) 6.54-6.68 (m, 1 H) 6.29-6.41 (m, 1 H) |
| | | 4.79-5.56 (m, 2 H) 3.79 (br d, J = 12.76 Hz, 2 H) 3.60 (s, 3 |
| | | H) 3.52 (br d, J = 11.38 Hz, 4 H) 3.08-3.25 (m, 4 H) 2.91- |
| | | 2.98 (m, 2 H) 2.87 (br s, 3 H) 1.97-2.16 (m, 3 H) |
| 029 | 581.5 | δ: 8.65 (s, 1H), 8.29 (s, 1H), 7.89-7.72 (m, 2H), 6.94-6.83 |
| | | (m, 1H), 6.69-6.60 (m, 2H), 6.55 (dd, J = 2.4, 8.9 Hz, 1H), |
| | | 6.35 (d, J = 8.0 Hz, 1H), 6.30-6.20 (m, 1H), 5.74-5.65 (m, |
| | | 1H), 3.85 (s, 3H), 3.56 (s, 4H), 3.19-3.14 (m, 4H), 2.47 (br |
| | | s, 4H), 2.25 (s, 3H) |
| 030 | 552.5 | δ: 9.73 (br s, 1H), 8.77 (s, 1H), 8.17-8.09 (m, 2H), 7.88 (s, |
| | | 1H), 7.57 (dd, J = 2.9, 9.1 Hz, 1H), 7.00-6.80 (m, 1H), 6.66 |
| | | (br d, J = 6.7 Hz, 1H), 6.41-6.21 (m, 3H), 5.76-5.62 (m, |
| | | 1H), 3.64 (s, 5H), 3.42-3.29 (m, 10H), 2.89 (s, 3H), 2.14 (s, 3H) |
| 031 | 552.4 | δ: 10.01 (br s, 1H), 9.92-9.75 (m, 1H), 8.73 (s, 1H), 8.61 |
| | | (br s, 1H), 8.03 (dd, J = 2.0, 9.0 Hz, 1H), 7.92 (s, 1H), 7.03 |
| | | (d, J = 9.1 Hz, 1H), 6.95-6.78 (m, 1H), 6.69-6.53 (m, 1H), |
| | | 6.35 (d, J = 8.0 Hz, 1H), 6.30-6.22 (m, 2H), 5.73 (dd, J = |
| | | 3.8, 8.8 Hz, 1H), 4.88 (br dd, J = 5.3, 12.4 Hz, 1H), 4.34 (br |
| | | d, J = 9.4 Hz, 4H), 3.71 (br dd, J = 4.1, 10.4 Hz, 1H), 3.63- |
| | | 3.55 (m, 3H), 3.52 (br s, 1H), 3.41 (dt, J = 5.5, 11.2 Hz, 1H), |
| | | 3.16-3.05 (m, 4H), 2.85 (br s, 3H), 2.15-2.01 (m, 3H) |
| 032 | 566.5 | δ: 9.95 (br s, 1H), 8.72 (s, 1H), 7.91 (s, 1H), 7.70 (br d, J = |
| | | 8.9 Hz, 2H), 6.99-6.86 (m, 3H), 6.65 (d, J = 7.4 Hz, 1H), |
| | | 6.35 (d, J = 8.0 Hz, 1H), 6.30-6.20 (m, 2H), 5.73 (dd, J = |
| | | 4.0, 8.5 Hz, 1H), 4.88 (dd, J = 4.7, 12.3 Hz, 1H), 4.05 (t, J = |
| | | 5.9 Hz, 2H), 3.70 (br dd, J = 4.6, 10.4 Hz, 1H), 3.62-3.56 |
| | | (m, 3H), 3.48-3.40 (m, 1H), 3.01 (dt, J = 4.5, 12.3 Hz, 1H), |
| | | 2.78 (t, J = 5.9 Hz, 2H), 2.54-2.51 (m, 4H), 2.12 (s, 3H), |
| | | 1.71-1.66 (m, 4H) |
| 033 | 540.4 | δ: 9.73 (s, 1H), 8.71 (s, 1H), 7.86 (s, 1H), 7.75 (d, J = 8.9 |
| | | Hz, 2H), 7.03 (d, J = 8.9 Hz, 2H), 6.89 (br t, J = 7.7 Hz, 1H), |
| | | 6.64 (br d, J = 7.2 Hz, 1H), 6.48-6.05 (m, 2H), 5.77-5.65 |
| | | (m, 1H), 4.91-4.51 (m, 4H), 4.34 (t, J = 5.1 Hz, 2H), 3.62 |
| | | (s, 3H), 3.55-3.51 (m, 2H), 2.91 (s, 6H), 2.14 (s, 3H) |
| 034 | 560.4 | δ: 9.89 (s, 1H), 8.75 (s, 1H), 7.92-7.77 (m, 3H), 7.36 (d, J = |
| | | 8.6 Hz, 2H), 6.89 (br t, J = 7.9 Hz, 1H), 6.65 (br d, J = 5.5 |
| | | Hz, 2H), 6.42-6.18 (m, 3H), 5.79-5.62 (m, 1H), 5.03-4.72 |
| | | (m, 1H), 4.26 (s, 2H), 3.79-3.41 (m, 5H), 3.03-2.89 (m, |
| | | 1H), 2.58 (d, J = 4.5 Hz, 3H), 2.14 (s, 3H) |
| 035 | 586.4 | δ: 10.13-10.09 (m, 1H), 8.77 (s, 1H), 7.94 (s, 1H), 7.68- |
| | | 7.62 (m, 2H), 7.17 (d, J = 8.2 Hz, 1H), 6.92 (t, J = 7.8 Hz, |
| | | 1H), 6.67 (d, J = 7.0 Hz, 1H), 6.37 (d, J = 8.8 Hz, 1H), 6.31- |
| | | 6.25 (m, 2H), 5.74 (dd, J = 4.0, 8.6 Hz, 1H), 4.94-4.85 (m, |
| | | 1H), 4.37 (s, 3H), 3.73 (br dd, J = 4.1, 10.0 Hz, 2H), 3.65- |
| | | 3.61 (m, 4H), 2.97 (s, 3H), 2.88 (br t, J = 5.7 Hz, 3H) |
| 036 | 569.6 | δ: 9.83-9.66 (m, 1H), 9.73 (br s, 1H), 9.47 (s, 1H), 8.68 (s, |
| | | 1H), 7.90 (s, 1H), 7.52 (br s, 1H), 6.98 (dd, J = 2.6, 13.8 Hz, |
| | | 1H), 6.93-6.82 (m, 2H), 6.65 (d, J = 7.4 Hz, 1H), 6.35 (d, J = |
| | | 8.1 Hz, 1H), 6.30-6.23 (m, 2H), 5.72 (dd, J = 3.6, 8.8 Hz, |
| | | 1H), 5.76-5.65 (m, 1H), 4.88 (br dd, J = 5.1, 12.5 Hz, 1H), |
| | | 3.89 (br d, J = 13.5 Hz, 2H), 3.70 (br dd, J = 4.6, 10.3 Hz, |
| | | 1H), 3.52 (br d, J = 12.0 Hz, 2H), 3.48 (s, 3H), 3.41 (s, 1H), |
| | | 3.16 (br d, J = 10.6 Hz, 2H), 3.06-2.93 (m, 3H), 2.87 (br d, |
| | | J = 2.8 Hz, 3H), 2.14-2.01 (m, 3H) |
| 037 | 514.3 | δ: 10.14-10.03 (m, 1H), 8.69 (br s, 1H), 8.11-8.00 (m, |
| | | 1H), 7.91 (s, 1H), 7.60 (br s, 1H), 6.90 (t, J = 7.9 Hz, 1H), |
| | | 6.64 (br d, J = 7.8 Hz, 1H), 6.39-6.19 (m, 3H), 5.73 (dd, J = |
| | | 4.0, 8.6 Hz, 1H), 4.88 (br dd, J = 5.3, 12.3 Hz, 1H), 4.19 (br |
| | | s, 2H), 3.71-3.60 (m, 3H), 3.48-3.38 (m, 2H), 3.01 (dt, J = |
| | | 4.5, 12.1 Hz, 1H), 2.63 (t, J = 6.3 Hz, 2H), 2.21-2.10 (m, 9H) |
| 038 | 486.2 | δ: 9.74 (br s, 1H), 8.68 (s, 1H), 7.99 (s, 1H), 7.84 (s, 1H), |
| | | 7.65 (s, 1H), 6.88 (br t, J = 7.8 Hz, 1H), 6.63 (br d, J = 7.2 |
| | | Hz, 1H), 6.49-6.10 (m, 3H), 5.71 (br d, J = 6.8 Hz, 1H), |
| | | 5.06-4.77 (m, 1H), 4.73-4.57 (m, 1H), 4.15 (t, J = 5.7 Hz, |
| | | 2H), 3.82-3.73 (m, 2H), 3.71-3.40 (m, 5H), 2.13 (s, 3H) |
| 039 | 540.4 | δ: 10.19-9.88 (m, 1H), 9.70-9.44 (m, 1H), 8.71 (br s, 1H), |
| | | 8.08 (br d, J = 13.4 Hz, 1H), 8.11-7.86 (m, 1H), 7.77-7.58 |
| | | (m, 1H), 6.93-6.77 (m, 1H), 6.64 (d, J = 7.3 Hz, 1H), 6.37- |
| | | 6.31 (m, 1H), 6.30-6.19 (m, 2H), 5.73 (dd, J = 3.6, 8.8 Hz, |
| | | 1H), 4.93-4.85 (m, 1H), 4.60-4.40 (m, 1H), 3.72 (br s, 1H), |
| | | 3.58 (br s, 1H), 3.18-3.07 (m, 2H), 3.07-2.94 (m, 1H), |
| | | 2.83 (br d, J = 4.3 Hz, 3H), 2.30-2.21 (m, 2H), 2.20-2.02 |
| | | (m, 5H) |
| 040 | 569.6 | δ: 10.03-9.78 (m, 1H), 8.75 (s, 1H), 7.96-7.84 (m, 1H), |
| | | 7.83-7.66 (m, 1H), 7.52 (br dd, J = 3.6, 4.6 Hz, 1H), 7.27- |
| | | 7.01 (m, 1H), 6.98-6.76 (m, 1H), 6.72-6.55 (m, 1H), 6.47- |
| | | 6.17 (m, 3H), 5.71 (br dd, J = 3.6, 5.7 Hz, 1H), 5.03-4.64 |
| | | (m, 1H), 3.91-3.71 (m, 1H), 3.64 (s, 3H), 3.60-3.49 (m, |
| | | 4H), 3.33-2.97 (m, 6H), 2.89 (s, 3H), 2.14 (s, 3H) |
| 041 | 549.5 | δ: 9.80 (br s, 1H), 8.69 (s, 1H), 8.06 (s, 1H), 7.85 (s, 1H), |
| | | 7.71 (s, 1H), 6.88 (br t, J = 7.8 Hz, 1H), 6.63 (br d, J = 7.5 |
| | | Hz, 1H), 6.40-6.15 (m, 3H), 5.76-5.66 (m, 1H), 5.04-4.72 |
| | | (m, 1H), 4.61-4.52 (m, 2H), 3.75-3.66 (m, 4H), 3.65 (s, |
| | | 3H), 3.21-2.94 (m, 1H), 2.88 (s, 3H), 2.13 (s, 3H) |
| 042 | 596.4 | δ: 9.79 (br s, 1H), 8.73-8.62 (m, 1H), 8.19 (s, 1H), 7.84 (s, |
| | | 1H), 7.67-7.53 (m, 2H), 6.98-6.86 (m, 3H), 6.65 (br d, J = |
| | | 7.5 Hz, 1H), 6.39-6.12 (m, 3H), 5.86-5.70 (m, 1H), 5.07- |
| | | 4.82 (m, 2H), 4.03 (t, J = 5.8 Hz, 2H), 3.93-3.83 (m, 1H), |
| | | 3.70 (br dd, J = 3.8, 10.6 Hz, 1H), 3.42 (dt, J = 5.4, 11.1 Hz, |
| | | 2H), 3.19-3.08 (m, 1H), 3.07-2.93 (m, 1H), 2.77 (br d, J = |
| | | 7.3 Hz, 2H), 2.62 (br t, J = 5.8 Hz, 2H), 2.23 (s, 6H), 2.13 (s, 3H) |
| | | (VTNMR @353K) δ: 9.50 (s, 1H), 8.65 (s, 1H), 8.17 (s, 1H), |
| | | 7.85-7.75 (m, 1H), 7.60 (br d, J = 8.9 Hz, 2H), 7.00-6.85 |
| | | (m, 3H), 6.63 (br d, J = 6.8 Hz, 1H), 6.44-6.20 (m, 3H), |
| | | 5.78-5.66 (m, 1H), 5.06-4.93 (m, 1H), 4.91-4.70 (m, 1H), |
| | | 4.06 (t, J = 5.8 Hz, 2H), 3.90 (br t, J = 7.2 Hz, 1H), 3.73- |
| | | 3.42 (m, 3H), 3.23-3.16 (m, 1H), 2.85-2.73 (m, 2H), 2.73- |
| | | 2.60 (m, 4H), 2.25 (s, 6H), 2.18-2.09 (m, 3H) |
| 043 | 596.4 | δ: 9.79 (br s, 1H), 8.68 (s, 1H), 7.84 (s, 1H), 7.61 (d, J = 9.0 |
| | | Hz, 2H), 7.04-6.86 (m, 3H), 6.70-6.52 (m, 1H), 6.42-6.21 |
| | | (m, 3H), 5.74 (dd, J = 3.9, 8.4 Hz, 1H), 5.15-4.82 (m, 3H), |
| | | 4.07-3.98 (m, 2H), 3.94-3.82 (m, 1H), 3.70 (br dd, J = 3.8, |
| | | 10.7 Hz, 1H), 3.42 (dt, J = 5.5, 11.2 Hz, 2H), 3.05-2.94 (m, |
| | | 1H), 2.77 (br d, J = 6.4 Hz, 2H), 2.68-2.58 (m, 4H), 2.22 (s, |
| | | 6H), 2.13 (s, 3H) |
| | | (VTNMR @353K) δ: 9.51 (s, 1H), 8.66 (s, 1H), 7.79 (s, 1H), |
| | | 7.61 (br d, J = 8.9 Hz, 2H), 7.01-6.85 (m, 3H), 6.64 (br d, J = |
| | | 7.3 Hz, 1H), 6.43-6.19 (m, 3H), 5.80-5.62 (m, 1H), 5.07- |
| | | 4.71 (m, 3H), 4.11-4.04 (m, 2H), 3.91 (br d, J = 6.6 Hz, |
| | | 1H), 3.77-3.63 (m, 1H), 3.55-3.41 (m, 1H), 2.85-2.75 (m, |
| | | 2H), 2.73-2.62 (m, 4H), 2.26 (s, 6H), 2.14 (br s, 3H) |
| 044 | 596.4 | δ: 9.82 (br s, 1H), 8.73-8.65 (m, 1H), 7.89-7.81 (m, 1H), |
| | | 7.64-7.54 (m, 2H), 6.98-6.88 (m, 3H), 6.64 (br d, J = 7.4 |
| | | Hz, 1H), 6.35-6.27 (m, 2H), 6.24-6.14 (m, 1H), 5.84-5.68 |
| | | (m, 1H), 4.99 (br d, J = 3.9 Hz, 1H), 4.87 (br dd, J = 4.6, |
| | | 12.8 Hz, 1H), 4.36 (br s, 1H), 4.03 (t, J = 5.8 Hz, 2H), 3.69 |
| | | (br dd, J = 4.3, 10.4 Hz, 1H), 3.54-3.38 (m, 3H), 3.20-3.07 |
| | | (m, 2H), 3.05-2.94 (m, 1H), 2.62 (t, J = 5.8 Hz, 2H), 2.22 |
| | | (s, 6H), 2.12 (s, 3H) |
| | | (VTNMR @353K) δ: 9.53 (s, 1H), 8.66 (s, 1H), 7.79 (s, 1H), |
| | | 7.60 (br d, J = 8.8 Hz, 2H), 6.99-6.84 (m, 3H), 6.69-6.58 |
| | | (m, 1H), 6.36-6.24 (m, 2H), 6.15 (br t, J = 8.5 Hz, 1H), 5.75- |
| | | 5.66 (m, 1H), 5.03-4.78 (m, 1H), 4.73 (br d, J = 1.5 Hz, |
| | | 1H), 4.41 (br s, 1H), 4.06 (br t, J = 5.8 Hz, 2H), 3.77-3.39 |
| | | (m, 2H), 3.24-3.09 (m, 3H), 2.65 (br t, J = 5.8 Hz, 2H), 2.25 |
| | | (s, 6H), 2.21-2.10 (m, 5H) |
| 045 | 586.4 | δ: 9.68 (s, 1H), 8.74 (s, 1H), 7.87 (s, 1H), 7.54 (s, 1H), 7.34 |
| | | (br d, J = 8.2 Hz, 1H), 7.27-7.19 (m, 1H), 6.89 (br t, J = 7.7 |
| | | Hz, 1H), 6.74-6.62 (m, 2H), 6.42-6.20 (m, 3H), 5.71 (br d, |
| | | J = 6.8 Hz, 1H), 5.05-4.74 (m, 1H), 3.81-3.77 (m, 4H), |
| | | 3.76-3.67 (m, 1H), 3.64 (s, 3H), 3.56-3.44 (m, 1H), 3.16 |
| | | (br d, J = 4.8 Hz, 4H), 3.08-3.02 (m, 1H), 2.14 (s, 3H) |
| 046 | 607.4 | δ: 9.90-9.74 (m, 1H), 9.65 (br s, 1H), 8.76-8.61 (m, 1H), |
| | | 7.93-7.79 (m, 1H), 7.70-7.52 (m, 2H), 7.07-6.86 (m, 3H), |
| | | 6.65 (br d, J = 7.4 Hz, 1H), 6.39-6.18 (m, 3H), 5.74 (dd, J = |
| | | 3.9, 8.6 Hz, 1H), 5.09-4.95 (m, 1H), 4.89 (br dd, J = 4.7, |
| | | 12.4 Hz, 1H), 3.88 (s, 1H), 3.79 (br d, J = 12.8 Hz, 2H), 3.69 |
| | | (br d, J = 4.1 Hz, 1H), 3.53 (br d, J = 12.4 Hz, 2H), 3.43 (td, |
| | | J = 5.5, 11.2 Hz, 1H), 3.17 (br s, 3H), 2.92 (br s, 3H), 2.87 |
| | | (br d, J = 3.5 Hz, 3H), 2.79 (br d, J = 8.0 Hz, 2H), 2.65 (br d, |
| | | J = 7.1 Hz, 2H), 2.13 (s, 3H) |
| 047 | 570.4 | δ: 9.81-9.32 (m, 2H), 8.80-8.60 (m, 1H), 7.84 (s, 1H), 7.75 |
| | | (d, J = 8.9 Hz, 2H), 7.08-6.94 (m, 2H), 6.93-6.85 (m, 1H), |
| | | 6.69-6.59 (m, 1H), 6.40-6.32 (m, 1H), 6.29-6.21 (m, 1H), |
| | | 5.78-5.64 (m, 1H), 4.99-4.78 (m, 1H), 4.48-4.38 (m, 2H), |
| | | 4.37-4.29 (m, 2H), 3.74-3.66 (m, 3H), 3.56-3.49 (m, 3H), |
| | | 3.24 (br d, J = 4.1 Hz, 4H), 2.91 (s, 6H), 2.20-2.07 (m, 3H) |
| 048 | 600.3 | δ: 10.06 (s, 1H), 8.75 (s, 1H), 7.92 (s, 1H), 7.75 (d, J = 8.4 |
| | | Hz, 2H), 7.20 (d, J = 8.5 Hz, 2H), 6.91 (t, J = 7.8 Hz, 1H), |
| | | 6.65 (d, J = 7.5 Hz, 1H), 6.36 (d, J = 8.1 Hz, 1H), 6.33-6.19 |
| | | (m, 2H), 5.73 (dd, J = 4.0, 8.5 Hz, 1H), 4.92-4.86 (m, 1H), |
| | | 4.54 (br d, J = 11.4 Hz, 1H), 3.89 (br t, J = 7.8 Hz, 2H), 3.72 |
| | | (br dd, J = 4.1, 10.6 Hz, 1H), 3.65-3.62 (m, 2H), 3.61 (s, |
| | | 3H), 3.43 (td, J = 5.5, 11.4 Hz, 1H), 3.08-3.00 (m, 1H), 2.98 |
| | | (s, 3H), 2.84 (br s, 2H), 2.16-2.00 (m, 3H) |
| | | (VTNMR @353K) δ: 9.76 (s, 1H), 8.73 (s, 1H), 7.86 (s, 1H), |
| | | 7.75 (d, J = 8.5 Hz, 2H), 7.19 (d, J = 8.5 Hz, 2H), 6.89 (br t, |
| | | J = 7.8 Hz, 1H), 6.64 (br d, J = 7.4 Hz, 1H), 6.40-6.21 (m, |
| | | 3H), 5.70 (br dd, J = 3.9, 6.9 Hz, 1H), 4.98-4.78 (m, 2H), |
| | | 3.96-3.87 (m, 2H), 3.79-3.67 (m, 1H), 3.66-3.61 (m, 5H), |
| | | 3.57-3.42 (m, 1H), 2.93 (s, 3H), 2.91-2.84 (m, 3H), 2.14 |
| | | (s, 3H) |
| 049 | 602.3 | δ: 9.71 (s, 1H), 8.71 (s, 1H), 7.85 (s, 1H), 7.73 (d, J = 8.9 |
| | | Hz, 2H), 6.92-6.82 (m, 3H), 6.68-6.58 (m, 1H), 6.42-6.19 |
| | | (m, 3H), 5.76-5.62 (m, 1H), 5.13-4.92 (m, 2H), 4.92-4.79 |
| | | (m, 1H), 4.30 (dd, J = 6.4, 9.5 Hz, 2H), 3.91 (dd, J = 4.7, 9.6 |
| | | Hz, 2H), 3.67-3.66 (m, 1H), 3.62 (s, 3H), 3.56-3.42 (m, |
| | | 1H), 3.02 (s, 3H), 2.13 (s, 3H) |
| 050 | 565.5 | δ: 10.19 (br s, 1H), 8.78 (s, 1H), 7.94 (s, 1H), 7.84 (br d, J = |
| | | 8.5 Hz, 2H), 7.33 (br d, J = 7.4 Hz, 2H), 6.91 (br t, J = 7.8 |
| | | Hz, 1H), 6.67 (br d, J = 7.9 Hz, 1H), 6.37 (br d, J = 7.8 Hz, |
| | | 1H), 6.30-6.22 (m, 2H), 5.76-5.69 (m, 1H), 4.95-4.83 (m, |
| | | 2H), 3.74-3.71 (m, 2H), 3.64-3.60 (m, 6H), 3.07-3.00 (m, |
| | | 4H), 2.76 (br s, 2H), 2.67 (br s, 2H), 2.33 (br s, 2H), 2.13 (s, 3H) |
| 051 | 553.4 | δ: 9.98 (br s, 1H), 9.81-9.47 (m, 2H), 8.72 (s, 1H), 7.92 (s, |
| | | 1H), 7.71 (br d, J = 8.8 Hz, 2H), 7.02 (br d, J = 9.0 Hz, 2H), |
| | | 6.75 (t, J = 8.2 Hz, 1H), 6.56-6.33 (m, 1H), 6.28 (d, J = 7.6 |
| | | Hz, 1H), 6.19 (dd, J = 1.9, 16.8 Hz, 1H), 5.96 (d, J = 8.1 Hz, |
| | | 1H), 5.63 (br d, J = 10.5 Hz, 1H), 4.96-4.73 (m, 1H), 3.79 |
| | | (br d, J = 12.9 Hz, 3H), 3.60 (s, 3H), 3.52 (br d, J = 12.4 Hz, |
| | | 2H), 3.39 (br dd, J = 12.1, 16.4 Hz, 2H), 3.20-3.11 (m, 2H), |
| | | 2.97-2.91 (m, 2H), 2.87 (br d, J = 3.1 Hz, 3H) |
| 052 | 614.5 | δ: 9.61 (s, 1H), 8.77 (s, 1H), 8.70 (br d, J = 3.8 Hz, 1H), 8.58 |
| | | (d, J = 1.9 Hz, 1H), 7.98 (s, 1H), 7.87 (br d, J = 7.6 Hz, 1H), |
| | | 7.62 (dd, J = 5.0, 7.9 Hz, 1H), 7.24 (br d, J = 8.6 Hz, 2H), |
| | | 6.97-6.88 (m, 1H), 6.74-6.63 (m, 3H), 6.56 (br d, J = 7.6 |
| | | Hz, 1H), 6.28-6.19 (m, 1H), 5.70-5.63 (m, 1H), 3.78-3.69 |
| | | (m, 4H), 3.56-3.43 (m, 4H), 3.31-2.95 (m, 4H), 2.89 (s, |
| | | 3H), 2.13 (br s, 3H) |
| 053 | 562.4 | δ: 10.20 (br s, 1H), 9.69 (br d, J = 1.9 Hz, 1H), 8.78 (br s, |
| | | 1H), 8.00-7.78 (m, 3H), 7.34 (br s, 2H), 6.97-6.69 (m, 2H), |
| | | 6.53-6.38 (m, 1H), 6.33-6.14 (m, 2H), 5.98 (br d, J = 5.6 |
| | | Hz, 1H), 5.64 (br s, 1H), 4.84 (br s, 1H), 4.28 (br s, 2H), 3.63 |
| | | (br s, 4H), 3.02-2.89 (m, 1H), 2.57 (br s, 4H) |
| 054 | 569.4 | δ: 9.95 (s, 1H), 8.83 (s, 1H), 7.77-7.62 (m, 2H), 7.08-6.97 |
| | | (m, 2H), 6.90 (br t, J = 7.8 Hz, 1H), 6.69-6.57 (m, 1H), 6.48- |
| | | 6.09 (m, 3H), 5.72 (br d, J = 6.4 Hz, 1H), 5.02-4.63 (m, |
| | | 1H), 4.03-3.67 (m, 2H), 3.60 (s, 3H), 3.44 (br s, 6H), 3.31- |
| | | 3.04 (m, 4H), 2.88 (s, 3H), 2.19-2.00 (m, 3H) |
| 055 | 616.2 | δ: 9.93-9.81 (m, 1H), 8.72 (s, 1H), 7.89 (s, 1H), 7.58 (d, J = |
| | | 9.0 Hz, 2H), 6.98-6.88 (m, 3H), 6.66 (d, J = 7.4 Hz, 1H), |
| | | 6.41 (d, J = 8.3 Hz, 1H), 6.36-6.20 (m, 2H), 5.78-5.71 (m, |
| | | 1H), 5.70-5.57 (m, 1H), 4.88 (dd, J = 4.9, 12.6 Hz, 1H), |
| | | 4.03 (t, J = 5.8 Hz, 2H), 3.72 (br dd, J = 4.1, 10.0 Hz, 1H), |
| | | 3.64-3.41 (m, 3H), 3.07-2.82 (m, 4H), 2.61 (t, J = 5.8 Hz, |
| | | 2H), 2.21 (s, 6H), 2.13 (s, 3H) |
| 056 | 610.5 | δ: 10.05-9.80 (m, 1H), 9.62 (br s, 1H), 8.70 (s, 1H), 7.86 (s, |
| | | 1H), 7.69 (br d, J = 9.0 Hz, 2H), 7.00 (d, J = 9.0 Hz, 2H), |
| | | 6.95-6.79 (m, 1H), 6.70-6.51 (m, 1H), 6.39-6.16 (m, 3H), |
| | | 5.85-5.70 (m, 1H), 5.21-5.06 (m, 1H), 4.89 (br dd, J = 5.1, |
| | | 12.6 Hz, 1H), 4.29 (br t, J = 4.9 Hz, 2H), 3.90-3.77 (m, 1H), |
| | | 3.70 (br dd, J = 4.1, 10.8 Hz, 1H), 3.52 (br d, J = 4.4 Hz, |
| | | 2H), 3.47-3.39 (m, 1H), 3.00-2.91 (m, 2H), 2.87 (d, J = 4.3 |
| | | Hz, 6H), 2.40-2.28 (m, 2H), 2.16-2.03 (m, 3H), 1.32 (s, 3H) |
| 057 | 610.4 | δ: 9.62 (s, 1H), 8.67 (s, 1H), 7.80 (s, 1H), 7.73-7.66 (m, |
| | | 2H), 7.01 (d, J = 9.1 Hz, 2H), 6.89 (br t, J = 7.7 Hz, 1H), |
| | | 6.63 (br d, J = 7.2 Hz, 1H), 6.35 (br d, J = 8.1 Hz, 1H), 6.30- |
| | | 6.21 (m, 1H), 6.00 (t, J = 8.9 Hz, 1H), 5.71 (br d, J = 5.8 Hz, |
| | | 1H), 5.24-4.69 (m, 1H), 4.33 (t, J = 5.1 Hz, 2H), 3.77-3.59 |
| | | (m, 3H), 3.53 (t, J = 5.1 Hz, 3H), 2.99-2.85 (m, 8H), 2.34- |
| | | 2.22 (m, 2H), 2.13 (s, 3H), 1.39-1.25 (m, 3H) |
| 058 | 598.3 | δ: 9.61 (s, 1H), 8.71 (s, 1H), 7.87 (s, 1H), 7.74-7.54 (m, |
| | | 2H), 6.96-6.85 (m, 3H), 6.63 (br d, J = 6.9 Hz, 1H), 6.43- |
| | | 6.19 (m, 3H), 5.74-5.67 (m, 1H), 4.90 (br s, 1H), 4.46 (s, |
| | | 2H), 4.21 (s, 1H), 4.06 (t, J = 5.8 Hz, 2H), 3.78-3.32 (m, |
| | | 2H), 3.07-3.06 (m, 1H), 2.64 (t, J = 5.9 Hz, 2H), 2.25 (s, |
| | | 6H), 2.13 (s, 3H), 1.13 (s, 6H) |
| 059 | 605.4 | δ: 9.91 (s, 1 H) 8.82 (s, 1 H) 7.62 (s, 2 H) 6.84-7.08 (m, 3 |
| | | H) 6.23-6.50 (m, 2 H) 5.78 (br d, J = 12.40 Hz, 1 H) 3.57 (s, |
| | | 3 H) 2.89 (s, 3 H) 2.03 (s, 3 H) |
| 060 | 582.4 | δ: 9.64-9.56 (m, 1H), 8.67 (br d, J = 1.4 Hz, 1H), 8.14 (s, |
| | | 1H), 7.84 (s, 1H), 7.69-7.54 (m, 2H), 6.98-6.86 (m, 3H), |
| | | 6.64 (br d, J = 6.7 Hz, 1H), 6.42-6.33 (m, 1H), 6.31-6.23 |
| | | (m, 1H), 5.87-5.68 (m, 2H), 4.88-4.79 (m, 1H), 4.60 (dd, J = |
| | | 4.8, 11.0 Hz, 1H), 4.07 (t, J = 5.7 Hz, 2H), 4.01-3.91 (m, |
| | | 2H), 3.78-3.42 (m, 2H), 3.28-3.17 (m, 2H), 2.66 (t, J = 5.7 |
| | | Hz, 2H), 2.26 (s, 6H), 2.14 (s, 3H) |
| 061 | 540.4 | δ: 9.54 (s, 1H), 8.70 (s, 1H), 7.90 (s, 1H), 7.41 (br d, J = 7.5 |
| | | Hz, 1H), 7.02 (d, J = 11.5 Hz, 1H), 6.90 (t, J = 7.8 Hz, 1H), |
| | | 6.65 (d, J = 7.5 Hz, 1H), 6.42-6.30 (m, 1H), 6.30-6.20 (m, |
| | | 2H), 5.72 (dd, J = 4.0, 8.5 Hz, 1H), 4.88 (br dd, J = 5.0, 12.5 |
| | | Hz, 1H), 3.73-3.68 (m, 1H), 3.51-3.48 (m, 3H), 3.45 (s, |
| | | 2H), 3.42 (br s, 1H), 3.06-2.97 (m, 1H), 2.81 (br t, J = 5.5 |
| | | Hz, 2H), 2.58 (br t, J = 5.8 Hz, 2H), 2.34 (s, 3H), 2.17-2.01 |
| | | (m, 3H) |
| 062 | 484.2 | δ: 10.29 (s, 1H), 8.84 (s, 1H), 8.04 (d, J = 6.0 Hz, 1H), 7.90 |
| | | (s, 1H), 7.46 (s, 1H), 7.41 (d, J = 6.0 Hz, 1H), 6.95-6.87 (m, |
| | | 1H), 6.68 (br d, J = 6.6 Hz, 1H), 6.40 (d, J = 8.2 Hz, 1H), |
| | | 6.30-6.20 (m, 1H), 5.75-5.64 (m, 1H), 4.72-4.52 (m, 1H), |
| | | 3.90 (s, 3H), 3.81-3.70 (m, 1H), 3.68 (s, 3H), 3.60-3.37 |
| | | (m, 1H), 3.24-2.93 (m, 1H), 2.14 (s, 3H) |
| 063 | 498.3 | δ: 9.23-9.40 (m, 1 H) 8.85 (s, 1 H) 7.96 (d, J = 1.50 Hz, 2 H) |
| | | 7.66 (s, 1 H) 6.88-6.97 (m, 1 H) 6.69 (d, J = 7.50 Hz, 1 H) |
| | | 6.39 (d, J = 8.00 Hz, 1 H) 6.19-6.30 (m, 2 H) 5.73 (dd, |
| | | J = 8.38, 4.13 Hz, 1 H) 4.90 (dd, J = 12.63, 5.25 Hz, 1 H) 3.86 |
| | | (s, 3 H) 3.78 (br d, J = 3.88 Hz, 1 H) 3.60-3.62 (m, 3 H) 3.40 |
| | | (br s, 1 H) 3.04 (td, J = 12.04, 4.69 Hz, 1 H) 2.25 (s, 3 H) 2.13 |
| | | (s, 3 H) |
| 064 | 528.3 | δ: 9.92-9.81 (m, 1H), 9.75-9.64 (m, 1H), 9.59-9.51 (m, |
| | | 1H), 8.76-8.61 (m, 1H), 7.70 (br d, J = 8.4 Hz, 2H), 7.39 (s, |
| | | 1H), 7.13-6.93 (m, 3H), 6.69 (br d, J = 8.1 Hz, 1H), 6.59- |
| | | 6.42 (m, 2H), 6.16 (br d, J = 16.9 Hz, 1H), 5.68 (br d, J = |
| | | 10.0 Hz, 1H), 3.78 (br d, J = 13.0 Hz, 2H), 3.63 (s, 3H), 3.52 |
| | | (br d, J = 11.8 Hz, 2H), 3.22-3.11 (m, 2H), 3.00-2.90 (m, |
| | | 2H), 2.87 (br s, 3H) |
| 065 | 552.2 | δ: 9.56 (s, 1H), 8.66 (s, 1H), 8.20 (s, 1H), 7.78 (s, 1H), 7.64 |
| | | (d, J = 8.9 Hz, 2H), 6.92 (d, J = 9.1 Hz, 2H), 6.68 (t, J = 8.0 |
| | | Hz, 1H), 6.62-6.48 (m, 1H), 6.30-6.11 (m, 2H), 5.77 (d, J = |
| | | 7.6 Hz, 1H), 5.65 (br d, J = 8.9 Hz, 1H), 4.87-4.57 (m, |
| | | 2H), 3.61 (s, 3H), 3.14-3.10 (m, 4H), 3.08-2.99 (m, 2H), |
| | | 2.47 (br d, J = 5.0 Hz, 4H), 2.24 (s, 3H) |
| 066 | 542.3 | δ: 10.04 (br s, 1H), 9.76-9.54 (m, 2H), 8.74 (s, 1H), 7.93 (s, |
| | | 1H), 7.76 (br d, J = 8.9 Hz, 2H), 7.02 (d, J = 9.0 Hz, 2H), |
| | | 6.75 (t, J = 8.2 Hz, 1H), 6.53-6.38 (m, 1H), 6.28 (d, J = 7.4 |
| | | Hz, 1H), 6.19 (dd, J = 2.1, 16.9 Hz, 1H), 5.96 (dd, J = 0.8, |
| | | 8.3 Hz, 1H), 5.63 (br d, J = 9.9 Hz, 1H), 4.93-4.74 (m, 1H), |
| | | 4.30 (t, J = 5.0 Hz, 2H), 3.60 (s, 3H), 3.51 (br d, J = 4.6 Hz, |
| | | 3H), 3.45-3.32 (m, 1H), 3.03-2.91 (m, 1H), 2.87 (d, J = 4.4 |
| | | Hz, 6H) |
| 067 | 603.3 | δ: 10.11-9.78 (m, 1H), 8.79 (s, 1H), 8.73 (br d, J = 3.5 Hz, |
| | | 1H), 8.59 (d, J = 2.0 Hz, 1H), 8.26-8.14 (m, 1H), 8.03 (s, |
| | | 1H), 7.90 (br d, J = 8.5 Hz, 1H), 7.65 (dd, J = 4.5, 8.0 Hz, |
| | | 1H), 7.21 (br d, J = 4.0 Hz, 2H), 7.00-6.89 (m, 1H), 6.71- |
| | | 6.64 (m, 1H), 6.58 (br d, J = 8.0 Hz, 3H), 6.30-6.13 (m, |
| | | 2H), 5.69 (dd, J = 3.0, 9.5 Hz, 1H), 4.90 (br dd, J = 5.5, 12.5 |
| | | Hz, 1H), 3.95 (t, J = 5.8 Hz, 2H), 3.79 (br dd, J = 4.5, 10.0 |
| | | Hz, 1H), 3.51-3.46 (m, 1H), 3.09-2.96 (m, 1H), 2.59 (t, J = |
| | | 5.8 Hz, 2H), 2.22 (s, 6H), 2.11 (s, 3H) |
| 068 | 570.2 | δ: 9.74-9.41 (m, 1H), 8.68 (s, 1H), 8.43 (s, 1H), 7.94-7.80 |
| | | (m, 2H), 6.89 (br t, J = 7.7 Hz, 1H), 6.75 (d, J = 2.1 Hz, 1H), |
| | | 6.71-6.61 (m, 2H), 6.40-6.20 (m, 3H), 5.77-5.62 (m, 1H), |
| | | 4.89 (br s, 1H), 4.37 (t, J = 5.0 Hz, 2H), 3.87 (s, 3H), 3.77- |
| | | 3.62 (m, 1H), 3.56 (s, 3H), 3.55-3.51 (m, 2H), 3.51-3.40 |
| | | (m, 1H), 2.96-2.95 (m, 1H), 2.92 (s, 5H), 2.13 (s, 3H) |
| 069 | 509.2 | δ: 9.99-9.90 (m, 1H), 8.73 (s, 1H), 7.86 (s, 1H), 7.76 (d, J = |
| | | 7.8 Hz, 2H), 7.35 (t, J = 7.9 Hz, 2H), 7.03 (t, J = 7.4 Hz, 1H), |
| | | 6.92 (t, J = 7.9 Hz, 1H), 6.66 (d, J = 7.4 Hz, 1H), 6.41-6.32 |
| | | (m, 1H), 6.32-6.26 (m, 2H), 5.74 (dd, J = 4.1, 8.4 Hz, 1H), |
| | | 5.15-4.95 (m, 2H), 4.89 (br dd, J = 4.9, 12.6 Hz, 1H), 3.96- |
| | | 3.81 (m, 1H), 3.72 (br dd, J = 3.8, 10.7 Hz, 1H), 3.43 (dt, J = |
| | | 5.3, 11.1 Hz, 1H), 3.00 (dt, J = 4.6, 12.1 Hz, 1H), 2.85-2.73 |
| | | (m, 2H), 2.72-2.59 (m, 2H), 2.19-1.95 (m, 3H) |
| | | (VTNMR @353K) δ: 9.65 (s, 1H), 8.70 (s, 1H), 7.80 (s, 1H), |
| | | 7.76 (s, 1H), 7.74 (s, 1H), 7.35 (t, J = 7.9 Hz, 2H), 7.05 (t, J = |
| | | 7.3 Hz, 1H), 6.90 (br t, J = 7.7 Hz, 1H), 6.64 (br d, J = 7.2 |
| | | Hz, 1H), 6.37 (br d, J = 8.1 Hz, 1H), 6.31-6.21 (m, 1H), |
| | | 5.78-5.65 (m, 1H), 5.08-4.95 (m, 1H), 4.95-4.84 (m, 1H), |
| | | 4.80 (d, J = 6.7 Hz, 1H), 3.98-3.84 (m, 1H), 3.79-3.39 (m, |
| | | 2H), 3.08-3.06 (m, 1H), 2.87-2.63 (m, 4H), 2.14 (s, 3H) |
| 070 | 509.2 | δ: 9.99 (s, 1H), 8.74 (s, 1H), 7.88 (s, 1H), 7.75 (d, J = 7.8 |
| | | Hz, 2H), 7.35 (t, J = 7.9 Hz, 2H), 7.05 (t, J = 7.4 Hz, 1H), |
| | | 6.91 (t, J = 7.9 Hz, 1H), 6.65 (d, J = 7.4 Hz, 1H), 6.36 (d, J = |
| | | 8.1 Hz, 1H), 6.31-6.26 (m, 2H), 6.25-6.16 (m, 1H), 5.74 |
| | | (dd, J = 4.2, 8.3 Hz, 1H), 5.01 (br d, J = 3.9 Hz, 1H), 4.88 (br |
| | | dd, J = 4.6, 12.4 Hz, 1H), 4.41 (br s, 1H), 3.71 (br dd, J = |
| | | 4.4, 10.2 Hz, 1H), 3.42 (dt, J = 5.5, 11.2 Hz, 1H), 3.22-3.09 |
| | | (m, 2H), 3.00 (dt, J = 4.5, 12.1 Hz, 1H), 2.21-2.00 (m, 6H) |
| 071 | 610.3 | δ: 9.89 (br s, 1H), 9.70 (br s, 1H), 8.76-8.64 (m, 1H), 7.92- |
| | | 7.83 (m, 1H), 7.67 (d, J = 9.0 Hz, 2H), 7.02 (d, J = 9.1 Hz, |
| | | 2H), 6.96-6.79 (m, 1H), 6.66 (br d, J = 7.4 Hz, 1H), 6.40- |
| | | 6.22 (m, 3H), 5.88-5.69 (m, 1H), 5.27-5.07 (m, 1H), 4.89 |
| | | (br dd, J = 5.2, 12.2 Hz, 1H), 4.30 (t, J = 4.9 Hz, 2H), 3.75- |
| | | 3.62 (m, 2H), 3.52 (br d, J = 4.3 Hz, 2H), 3.43 (td, J = 5.4, |
| | | 11.3 Hz, 1H), 3.19-3.13 (m, 3H), 3.08-2.95 (m, 1H), 2.88 |
| | | (d, J = 3.9 Hz, 6H), 2.86-2.75 (m, 2H), 2.72-2.59 (m, 2H), |
| | | 2.17-2.00 (m, 3H) |
| | | (VTNMR @353K) δ: 9.64-9.54 (m, 1H), 8.74-8.61 (m, |
| | | 1H), 7.84-7.76 (m, 1H), 7.72-7.61 (m, 2H), 7.02 (d, J = 8.9 |
| | | Hz, 2H), 6.90 (br t, J = 7.6 Hz, 1H), 6.64 (br d, J = 7.5 Hz, |
| | | 1H), 6.36 (br d, J = 7.9 Hz, 1H), 6.28 (br s, 1H), 5.77-5.64 |
| | | (m, 1H), 5.19-5.02 (m, 1H), 4.33 (t, J = 5.1 Hz, 2H), 3.68 |
| | | (td, J = 7.0, 13.9 Hz, 2H), 3.53 (t, J = 5.0 Hz, 3H), 3.21- |
| | | 3.15 (m, 3H), 3.14-2.98 (m, 1H), 2.91 (s, 6H), 2.88-2.77 |
| | | (m, 2H), 2.75-2.63 (m, 2H), 2.36-2.24 (m, 1H), 2.14 (s, 3H) |
| 072 | 596.3 | δ: 9.96 (br s, 1H), 9.68 (br s, 1H), 8.74 (s, 1H), 7.91 (s, 1H), |
| | | 7.67 (br d, J = 8.8 Hz, 2H), 7.03 (br d, J = 8.8 Hz, 2H), 6.92 |
| | | (br t, J = 7.8 Hz, 1H), 6.66 (br d, J = 7.3 Hz, 1H), 6.37 (br d, |
| | | J = 8.1 Hz, 1H), 6.32-6.22 (m, 2H), 6.19-6.07 (m, 1H), |
| | | 5.74 (br dd, J = 3.6, 8.6 Hz, 1H), 4.89 (br dd, J = 4.9, 12.2 |
| | | Hz, 1H), 4.39-4.24 (m, 2H), 4.04 (br d, J = 6.5 Hz, 1H), |
| | | 3.97-3.80 (m, 3H), 3.71 (br d, J = 7.3 Hz, 1H), 3.57-3.38 |
| | | (m, 3H), 3.08-2.95 (m, 1H), 2.88 (br d, J = 2.9 Hz, 6H), |
| | | 2.45-2.39 (m, 1H), 2.22-1.99 (m, 4H) |
| | | (VTNMR @353K) δ: 9.67 (s, 1H), 8.71 (s, 1H), 7.84 (s, 1H), |
| | | 7.68-7.63 (m, 2H), 7.02 (d, J = 9.1 Hz, 2H), 6.89 (br t, J = |
| | | 7.9 Hz, 1H), 6.64 (br d, J = 7.4 Hz, 1H), 6.36 (br d, J = 8.2 |
| | | Hz, 1H), 6.29-6.23 (m, 1H), 6.17-6.05 (m, 1H), 5.78-5.66 |
| | | (m, 1H), 5.02-4.74 (m, 1H), 4.34 (t, J = 5.1 Hz, 2H), 4.06 |
| | | (q, J = 7.5 Hz, 1H), 4.02-3.96 (m, 1H), 3.94-3.81 (m, 2H), |
| | | 3.76-3.61 (m, 1H), 3.60-3.39 (m, 3H), 3.26-2.98 (m, 1H), |
| | | 2.91 (s, 7H), 2.49-2.43 (m, 1H), 2.13 (s, 4H) |
| 073 | 525.3 | δ: 10.22-10.07 (m, 1H), 8.87-8.60 (m, 1H), 8.02-7.80 (m, |
| | | 1H), 7.38 (br s, 1H), 6.91 (t, J = 7.9 Hz, 1H), 6.75 (d, J = 0.9 |
| | | Hz, 1H), 6.65 (d, J = 7.4 Hz, 1H), 6.36 (d, J = 8.3 Hz, 1H), |
| | | 6.30-6.25 (m, 2H), 5.74 (dd, J = 3.9, 8.6 Hz, 1H), 4.96- |
| | | 4.83 (m, 1H), 4.22-4.07 (m, 2H), 3.46-3.39 (m, 2H), 3.09- |
| | | 2.95 (m, 4H), 2.13 (s, 3H) |
| 074 | 556.3 | δ: 10.04 (br s, 1H), 9.66 (br s, 1H), 8.74 (s, 1H), 7.96 (s, |
| | | 1H), 7.76 (br d, J = 8.9 Hz, 2H), 7.03 (d, J = 9.0 Hz, 2H), |
| | | 6.91 (t, J = 8.3 Hz, 1H), 6.45 (d, J = 7.9 Hz, 1H), 6.37-6.26 |
| | | (m, 1H), 6.21-6.11 (m, 2H), 5.66-5.59 (m, 1H), 4.89-4.74 |
| | | (m, 1H), 4.30 (t, J = 4.9 Hz, 2H), 3.72 (s, 3H), 3.61 (s, 3H), |
| | | 3.51 (br d, J = 4.0 Hz, 3H), 3.41 (br dd, J = 4.0, 10.9 Hz, |
| | | 1H), 2.87 (br d, J = 3.5 Hz, 7H) |
| 075 | 558.3 | δ: 10.18 (s, 1H), 9.78 (br s, 1H), 8.78 (s, 1H), 7.99-7.76 (m, |
| | | 2H), 7.56 (br d, J = 9.0 Hz, 1H), 7.27 (t, J = 9.3 Hz, 1H), |
| | | 7.01-6.81 (m, 1H), 6.66 (d, J = 7.4 Hz, 1H), 6.52-6.10 (m, |
| | | 3H), 5.73 (dd, J = 3.9, 8.6 Hz, 1H), 4.89 (br dd, J = 5.1, 12.4 |
| | | Hz, 1H), 4.38 (t, J = 4.9 Hz, 2H), 3.73 (br dd, J = 4.1, 10.4 |
| | | Hz, 1H), 3.65-3.59 (m, 3H), 3.54 (br s, 2H), 3.42 (dt, J = |
| | | 5.5, 11.2 Hz, 1H), 3.07-2.99 (m, 1H), 2.89 (br s, 6H), 2.19- |
| | | 1.95 (m, 3H) |
| 076 | 538.4 | δ: 10.04 (br s, 1H), 8.74 (s, 1H), 7.92 (s, 1H), 7.77 (br d, J = |
| | | 8.3 Hz, 2H), 6.90 (br d, J = 7.0 Hz, 3H), 6.69-6.62 (m, 1H), |
| | | 6.36 (br d, J = 8.1 Hz, 1H), 6.30-6.20 (m, 2H), 5.78-5.66 |
| | | (m, 1H), 4.88 (br dd, J = 5.0, 12.4 Hz, 1H), 4.79-4.67 (m, |
| | | 1H), 4.46-4.35 (m, 1H), 4.31-4.22 (m, 1H), 4.13-3.99 (m, |
| | | 2H), 3.54-3.38 (m, 5H), 2.94-2.89 (m, 2H), 2.49-2.42 (m, |
| | | 3H), 2.13 (s, 3H) |
| 077 | 612.4 | δ: 9.90 (br d, J = 11.4 Hz, 1H), 9.70 (br s, 1H), 8.74-8.66 |
| | | (m, 1H), 7.94-7.86 (m, 1H), 7.72-7.60 (m, 2H), 7.02 (d, J = |
| | | 9.1 Hz, 2H), 6.96-6.87 (m, 1H), 6.45 (d, J = 8.3 Hz, 1H), |
| | | 6.39-6.27 (m, 1H), 6.20 (d, J = 2.4 Hz, 1H), 6.16-6.11 (m, |
| | | 1H), 5.68-5.58 (m, 1H), 5.09-4.94 (m, 1H), 4.82 (br d, J = |
| | | 2.6 Hz, 1H), 4.31 (br t, J = 4.9 Hz, 2H), 3.88 (br t, J = 7.2 |
| | | Hz, 1H), 3.72 (s, 3H), 3.66 (br d, J = 10.1 Hz, 1H), 3.52 (br |
| | | d, J = 4.4 Hz, 2H), 3.40 (br d, J = 4.3 Hz, 1H), 3.16 (s, 1H), |
| | | 3.02-2.92 (m, 1H), 2.88 (d, J = 4.1 Hz, 6H), 2.79 (br d, J = |
| | | 8.0 Hz, 1H), 2.65 (br d, J = 7.1 Hz, 1H), 2.13 (br t, J = 10.1 |
| | | Hz, 1H) |
| 078 | 542.4 | δ: 10.07 (br s, 1H), 9.62 (br s, 1H), 9.17-8.94 (m, 1H), 8.77 |
| | | (s, 1H), 7.98 (s, 1H), 7.76 (br d, J = 8.9 Hz, 2H), 7.03 (d, J = |
| | | 9.1 Hz, 2H), 6.86-6.58 (m, 2H), 6.25 (br d, J = 16.6 Hz, |
| | | 1H), 6.13 (dd, J = 2.4, 8.6 Hz, 1H), 5.89 (d, J = 2.4 Hz, 1H), |
| | | 5.83-5.69 (m, 1H), 4.31 (t, J = 5.0 Hz, 2H), 3.95 (br t, J = |
| | | 4.9 Hz, 2H), 3.63 (s, 3H), 3.52 (br t, J = 4.6 Hz, 4H), 2.88 (d, |
| | | J = 4.5 Hz, 6H) |
| 079 | 527.3 | δ: 9.85-9.27 (m, 1H), 8.62 (s, 1H), 8.15-8.09 (m, 1H), 7.91 |
| | | (s, 1H), 7.73 (dd, J = 2.6, 8.6 Hz, 1H), 7.00 (d, J = 8.8 Hz, |
| | | 1H), 6.94-6.76 (m, 2H), 6.63 (br d, J = 7.4 Hz, 1H), 6.49 (br |
| | | d, J = 8.1 Hz, 1H), 6.27-6.17 (m, 1H), 5.66 (br d, J = 4.0 |
| | | Hz, 1H), 4.95-4.74 (m, 1H), 4.72-4.60 (m, 2H), 3.80-3.63 |
| | | (m, 2H), 3.59-3.54 (m, 2H), 3.23-3.17 (m, 1H), 2.95-2.86 |
| | | (m, 6H), 2.11 (s, 3H) |
| 080 | 526.3 | δ: 9.79-9.66 (m, 1H), 8.67-8.54 (m, 1H), 7.93 (s, 1H), 7.23 |
| | | (br d, J = 8.8 Hz, 2H), 7.14-6.97 (m, 4H), 6.91 (br t, J = 7.9 |
| | | Hz, 1H), 6.62 (br d, J = 7.3 Hz, 1H), 6.50-6.44 (m, 1H), |
| | | 6.29-6.13 (m, 2H), 5.73-5.64 (m, 1H), 4.94-4.79 (m, 1H), |
| | | 4.35 (br s, 2H), 3.55 (br s, 3H), 3.43 (dt, J = 5.7, 11.0 Hz, |
| | | 2H), 2.88 (br d, J = 3.3 Hz, 6H), 2.12-2.05 (m, 1H), 2.14- |
| | | 1.98 (m, 2H) |
| 081 | 540.4 | δ: 8.62 (s, 1H), 7.88 (s, 1H), 7.27-7.20 (m, 3H), 7.13-7.09 |
| | | (m, 2H), 6.90 (br t, J = 7.8 Hz, 1H), 6.62 (br d, J = 7.1 Hz, |
| | | 1H), 6.46 (br d, J = 8.0 Hz, 1H), 6.22 (br d, J = 13.4 Hz, 2H), |
| | | 5.74-5.58 (m, 1H), 5.01-4.65 (m, 1H), 4.41-4.36 (m, 2H), |
| | | 3.58-3.43 (m, 6H), 2.91 (s, 6H), 2.65 (br s, 3H), 2.11 (s, 3H) |
| 082 | 566.4 | (298K) δ: 8.73-8.56 (m, 1H), 8.16 (s, 1H), 7.93 (s, 1H), |
| | | 7.77-7.41 (m, 1H), 7.17 (br d, J = 5.5 Hz, 2H), 7.01 (br d, J = |
| | | 8.9 Hz, 2H), 6.92 (t, J = 7.9 Hz, 1H), 6.63 (d, J = 7.4 Hz, |
| | | 1H), 6.51-6.36 (m, 1H), 6.30-6.13 (m, 2H), 5.68 (dd, J = |
| | | 3.0, 9.5 Hz, 1H), 4.87 (br dd, J = 4.9, 12.6 Hz, 1H), 4.08 (t, J = |
| | | 5.7 Hz, 2H), 3.71 (br dd, J = 3.6, 10.8 Hz, 1H), 3.39 (br s, |
| | | 2H), 2.98 (dt, J = 4.6, 12.1 Hz, 1H), 2.64 (t, J = 5.8 Hz, 2H), |
| | | 2.22 (s, 6H), 2.12-2.00 (m, 3H), 0.69-0.29 (m, 4H) |
| | | (353K) δ: 8.62 (s, 1H), 8.13 (s, 1H), 7.87 (s, 1H), 7.44 (br s, |
| | | 1H), 7.17 (d, J = 8.8 Hz, 2H), 7.05-6.99 (m, 2H), 6.90 (br t, |
| | | J = 7.9 Hz, 1H), 6.62 (br d, J = 7.3 Hz, 1H), 6.46 (d, J = 8.3 |
| | | Hz, 1H), 6.33-6.10 (m, 2H), 5.66 (br s, 1H), 4.98-4.77 (m, |
| | | 1H), 4.11 (t, J = 5.8 Hz, 2H), 3.76-3.60 (m, 1H), 3.59-3.41 |
| | | (m, 1H), 3.03-2.92 (m, 1H), 2.67 (t, J = 5.8 Hz, 2H), 2.61- |
| | | 2.52 (m, 1H), 2.26 (s, 6H), 2.11 (s, 3H), 0.56-0.37 (m, 4H) |
| 083 | 417.3 | δ: 8.60 (br d, J = 1.1 Hz, 1H), 8.10 (br s, 1H), 7.86 (s, 1H), |
| | | 6.89 (br t, J = 7.9 Hz, 1H), 6.63 (br d, J = 7.5 Hz, 1H), 6.37- |
| | | 6.20 (m, 3H), 5.72 (dd, J = 3.8, 8.6 Hz, 1H), 4.87 (br dd, J = |
| | | 4.9, 12.4 Hz, 1H), 3.79-3.28 (m, 5H), 3.00 (dt, J = 4.5, 12.1 |
| | | Hz, 1H), 2.85 (br d, J = 2.5 Hz, 1H), 2.21-2.02 (m, 3H), |
| | | 0.75 (br d, J = 5.3 Hz, 2H), 0.61-0.50 (m, 2H) |
| 084 | 523.4 | δ: 9.75-9.65 (m, 1H), 8.75-8.68 (m, 1H), 7.85-7.80 (m, |
| | | 1H), 7.77-7.72 (m, 2H), 7.36 (t, J = 7.9 Hz, 2H), 7.12-7.02 |
| | | (m, 1H), 6.91 (br t, J = 7.7 Hz, 1H), 6.65 (br d, J = 7.3 Hz, |
| | | 1H), 6.38 (br d, J = 8.1 Hz, 1H), 6.32-6.24 (m, 2H), 5.78- |
| | | 5.66 (m, 1H), 5.20-5.07 (m, 1H), 5.02-4.82 (m, 1H), 3.75- |
| | | 3.65 0(m, 2H), 3.57-3.45 (m, 1H), 3.21-3.17 (m, 3H), 2.89- |
| | | 2.79 (m, 2H), 2.77-2.65 (m, 2H), 2.38-2.26 (m, 1H), 2.15 |
| | | (s, 3H) |
| 085 | 511.4 | δ: 11.15 (s, 1H), 8.93 (s, 1H), 7.54-7.37 (m, 1H), 7.02 (s, |
| | | 1H), 6.61 (br d, J = 7.0 Hz, 1H), 6.52-6.36 (m, 3H), 6.32- |
| | | 6.21 (m, 1H), 5.85-5.74 (m, 1H), 4.95-4.79 (m, 2H), 4.53- |
| | | 4.36 (m, 2H), 3.74 (s, 3H), 3.68 (br s, 2H), 3.62 (br d, J = 5.0 |
| | | Hz, 2H), 3.39 (br d, J = 4.4 Hz, 2H), 2.94 (s, 3H), 2.11 (s, 3H) |
| 086 | 475.4 | δ: 8.54 (s, 1H), 7.73 (s, 1H), 6.87 (br t, J = 7.7 Hz, 1H), 6.61 |
| | | (br d, J = 7.3 Hz, 1H), 6.32-6.22 (m, 4H), 5.74-5.66 (m, |
| | | 1H), 5.29 (ddd, J = 3.8, 8.4, 11.8 Hz, 1H), 4.99-4.75 (m, |
| | | 1H), 3.71-3.35 (m, 3H), 3.27 (s, 4H), 3.12-2.87 (m, 1H), |
| | | 2.80-2.66 (m, 2H), 2.15-2.08 (m, 5H), 1.64-1.54 (m, 2H), |
| | | 1.31-1.16 (m, 2H) |
| 087 | 475.4 | δ: 8.56 (s, 1H), 7.83-7.76 (m, 1H), 7.51-7.14 (m, 2H), |
| | | 6.89 (t, J = 7.9 Hz, 1H), 6.65-6.50 (m, 1H), 6.35-6.16 (m, |
| | | 3H), 5.73 (dd, J = 3.6, 8.9 Hz, 1H), 5.34 (br dd, J = 3.6, 5.4 |
| | | Hz, 1H), 4.85 (br dd, J = 4.9, 12.8 Hz, 1H), 3.61 (br dd, J = |
| | | 3.8, 10.9 Hz, 1H), 3.48-3.33 (m, 1H), 2.97 (dt, J = 4.3, 12.1 |
| | | Hz, 1H), 2.77-2.56 (m, 2H), 2.15-1.99 (m, 3H), 1.63 (br d, |
| | | J = 11.9 Hz, 2H), 1.54-1.39 (m, 4H), 1.23 (br s, 3H) |
| | | δ: 8.54 (s, 1H), 7.74 (s, 1H), 6.87 (br t, J = 7.8 Hz, 1H), 6.60 |
| | | (br d, J = 7.1 Hz, 1H), 6.40-6.20 (m, 3H), 5.70 (br d, J = 6.6 |
| | | Hz, 1H), 5.42-5.25 (m, 1H), 4.99-4.76 (m, 1H), 3.70-3.34 |
| | | (m, 3H), 3.15-2.91 (m, 1H), 2.79-2.62 (m, 2H), 2.12 (s, |
| | | 3H), 1.71-1.61 (m, 2H), 1.57-1.42 (m, 4H), 1.27 (s, 3H) |
| 088 | 475.4 | δ: 8.53 (s, 1H), 7.76 (s, 1H), 7.17 (br s, 2H), 6.89 (t, J = 7.8 |
| | | Hz, 1H), 6.61 (d, J = 7.3 Hz, 1H), 6.32-6.17 (m, 3H), 5.73 |
| | | (dd, J = 4.1, 8.4 Hz, 1H), 5.39-5.21 (m, 1H), 4.86 (br dd, J = |
| | | 5.1, 12.7 Hz, 1H), 4.00 (s, 1H), 3.63 (br dd, J = 3.9, 10.7 |
| | | Hz, 1H), 3.47-3.36 (m, 1H), 3.05-2.82 (m, 3H), 2.14-2.01 |
| | | (m, 3H), 1.64 (br d, J = 12.9 Hz, 2H), 1.44-1.32 (m, 2H), |
| | | 1.32-1.22 (m, 2H), 1.12 (s, 3H) |
| | | δ: 8.52 (s, 1H), 7.72 (s, 1H), 6.87 (s, 3H), 6.60 (br d, J = 7.0 |
| | | Hz, 1H), 6.29-6.22 (m, 2H), 5.76-5.64 (m, 1H), 5.34-5.24 |
| | | (m, 1H), 4.86 (br s, 1H), 3.72 (s, 1H), 3.69-3.55 (m, 1H), |
| | | 3.53-3.38 (m, 1H), 3.04-2.86 (m, 3H), 2.15-2.08 (m, 3H), |
| | | 1.67 (br d, J = 12.6 Hz, 2H), 1.42 (dt, J = 4.0, 13.3 Hz, 2H), |
| | | 1.32 (br d, J = 11.6 Hz, 2H), 1.15 (s, 3H) |
| 089 | 524.3 | δ: 8.59 (s, 1H), 7.85 (s, 1H), 7.12-7.05 (m, 2H), 7.04-6.97 |
| | | (m, 2H), 6.90 (br t, J = 7.8 Hz, 1H), 6.62 (br d, J = 7.4 Hz, |
| | | 1H), 6.46 (br d, J = 8.3 Hz, 1H), 6.28-6.17 (m, 2H), 5.65 (br |
| | | d, J = 1.1 Hz, 1H), 4.64-4.51 (m, 1H), 3.83-3.73 (m, 4H), |
| | | 3.69 (br d, J = 2.9 Hz, 1H), 3.59-3.42 (m, 1H), 3.23-3.14 |
| | | (m, 4H), 3.10-2.91 (m, 1H), 2.11 (s, 3H) |
| 090 | 469.3 | δ: 8.60 (s, 1H), 7.87 (s, 1H), 7.39 (t, J = 8.0 Hz, 1H), 6.99 |
| | | (dd, J = 2.3, 8.8 Hz, 1H), 6.91 (br t, J = 7.8 Hz, 1H), 6.88- |
| | | 6.79 (m, 2H), 6.62 (br d, J = 7.5 Hz, 1H), 6.48 (d, J = 8.0 Hz, |
| | | 1H), 6.30-6.15 (m, 2H), 5.65 (br d, J = 2.0 Hz, 1H), 4.99- |
| | | 4.74 (m, 1H), 3.78 (s, 4H), 3.56-3.40 (m, 1H), 3.10-2.87 |
| | | (m, 1H), 2.11 (s, 3H) |
| 091 | 473.3 | δ: 8.63 (s, 1H), 7.93 (s, 1H), 7.58-7.45 (m, 3H), 7.32-7.26 |
| | | (m, 1H), 7.25-7.10 (m, 2H), 6.92 (t, J = 7.9 Hz, 1H), 6.64 |
| | | (d, J = 7.4 Hz, 1H), 6.51 (br d, J = 8.4 Hz, 1H), 6.30-6.12 |
| | | (m, 2H), 5.68 (dd, J = 2.8, 9.6 Hz, 1H), 4.87 (br dd, J = 5.1, |
| | | 12.3 Hz, 1H), 3.71 (br dd, J = 4.0, 11.0 Hz, 1H), 3.44 (dt, J = |
| | | 5.4, 11.1 Hz, 1H), 3.05-2.90 (m, 1H), 2.14-2.00 (m, 3H) |
| 092 | 537.3 | δ: 8.59 (s, 1H), 7.90 (s, 1H), 7.11-6.95 (m, 6H), 6.91 (t, J = |
| | | 7.9 Hz, 1H), 6.62 (d, J = 7.4 Hz, 1H), 6.44 (d, J = 8.0 Hz, |
| | | 1H), 6.29-6.13 (m, 2H), 5.74-5.58 (m, 1H), 4.86 (br dd, J = |
| | | 4.7, 12.9 Hz, 1H), 3.68 (br dd, J = 3.6, 10.6 Hz, 1H), 3.42 |
| | | (dt, J = 5.6, 11.3 Hz, 1H), 3.22-3.14 (m, 4H), 2.97 (dt, J = |
| | | 4.6, 12.3 Hz, 1H), 2.47-2.43 (m, 4H), 2.23 (s, 3H), 2.13- |
| | | 1.98 (m, 3H) |
| 093 | 511.3 | δ: 8.61 (s, 1H), 7.91 (s, 1H), 7.28-7.03 (m, 4H), 6.92-6.82 |
| | | (m, 3H), 6.63 (d, J = 7.4 Hz, 1H), 6.49-6.41 (m, 1H), 6.29- |
| | | 6.11 (m, 2H), 5.72-5.63 (m, 1H), 5.34-5.27 (m, 1H), 4.95 |
| | | (t, J = 6.6 Hz, 2H), 4.86 (br dd, J = 4.9, 12.7 Hz, 1H), 4.58- |
| | | 4.55 (m, 2H), 3.69 (br dd, J = 3.9, 10.7 Hz, 1H), 3.42 (td, J = |
| | | 5.6, 11.1 Hz, 1H), 2.97 (dt, J = 4.6, 12.1 Hz, 1H), 2.15-2.00 |
| | | (m, 3H) |
| 094 | 538.4 | δ: 10.13-10.00 (m, 1H), 8.64 (s, 1H), 7.96 (s, 1H), 7.62 (br |
| | | d, J = 8.1 Hz, 2H), 7.42 (br d, J = 8.3 Hz, 2H), 7.16-6.95 |
| | | (m, 2H), 6.91 (t, J = 7.9 Hz, 1H), 6.63 (d, J = 7.5 Hz, 1H), |
| | | 6.49 (d, J = 8.0 Hz, 1H), 6.29-6.13 (m, 2H), 5.68 (dd, J = |
| | | 2.4, 9.8 Hz, 1H), 4.87 (br dd, J = 5.3, 12.5 Hz, 1H), 4.42 (br |
| | | s, 2H), 3.99 (br d, J = 12.0 Hz, 2H), 3.72-3.64 (m, 4H), 3.27 |
| | | (br s, 2H), 3.19-3.13 (m, 2H), 2.97 (dt, J = 4.4, 12.1 Hz, |
| | | 1H), 2.10 (s, 3H) |
| 095 | 579.4 | δ: 9.87-9.28 (m, 1H), 8.62 (s, 1H), 7.87 (s, 1H), 7.51-7.34 |
| | | (m, 1H), 7.09 (d, J = 8.9 Hz, 2H), 6.93-6.84 (m, 3H), 6.62 |
| | | (br d, J = 6.9 Hz, 1H), 6.45 (br d, J = 8.0 Hz, 1H), 6.22 (br d, |
| | | J = 13.0 Hz, 2H), 5.69-5.62 (m, 1H), 5.00-4.76 (m, 1H), |
| | | 3.69 (br t, J = 7.3 Hz, 3H), 3.57-3.44 (m, 1H), 3.27 (t, J = |
| | | 7.3 Hz, 2H), 3.21-2.98 (m, 1H), 2.96 (s, 3H), 2.87 (s, 6H), |
| | | 2.59 (br s, 1H), 2.11 (s, 3H), 0.56-0.41 (m, 4H) |
| 096 | 473.3 | δ: 8.65-8.51 (m, 1H), 7.88-7.59 (m, 2H), 6.95-6.82 (m, |
| | | 1H), 6.61 (br d, J = 7.1 Hz, 1H), 6.33 (br d, J = 8.1 Hz, 1H), |
| | | 6.29-6.23 (m, 1H), 6.19-6.06 (m, 1H), 5.76-5.65 (m, 1H), |
| | | 5.02-4.69 (m, 1H), 4.20-4.13 (m, 1H), 4.10-4.05 (m, 1H), |
| | | 3.90-3.83 (m, 3H), 3.73-3.40 (m, 2H), 3.20-2.79 (m, 2H), |
| | | 2.63-2.52 (m, 1H), 2.17-2.01 (m, 4H), 0.81-0.69 (m, 2H), |
| | | 0.66-0.51 (m, 2H) |
| 097 | 487.3 | δ: 8.57 (s, 1H), 7.77 (s, 2H), 6.88 (br t, J = 7.6 Hz, 1H), 6.62 |
| | | (br d, J = 6.9 Hz, 1H), 6.31 (br d, J = 8.0 Hz, 1H), 6.28-6.22 |
| | | (m, 1H), 5.75-5.67 (m, 1H), 5.55 (ddd, J = 3.8, 8.1, 11.9 |
| | | Hz, 1H), 5.01-4.75 (m, 1H), 3.99 (br dd, J = 4.2, 11.2 Hz, |
| | | 2H), 3.64 (br d, J = 2.1 Hz, 1H), 3.57-3.35 (m, 4H), 3.15- |
| | | 2.96 (m, 3H), 2.82 (td, J = 3.4, 7.1 Hz, 1H), 2.13 (s, 3H), |
| | | 1.53 (br d, J = 10.3 Hz, 2H), 0.83-0.75 (m, 2H), 0.63-0.56 |
| | | (m, 2H) |
| 098 | 455.3 | δ: 9.91-9.33 (m, 1H), 8.62 (s, 1H), 7.91 (s, 1H), 7.40-7.03 |
| | | (m, 3H), 6.92 (t, J = 7.9 Hz, 1H), 6.80 (dd, J = 2.0, 8.1 Hz, |
| | | 1H), 6.73-6.59 (m, 3H), 6.47 (d, J = 8.1 Hz, 1H), 6.30- |
| | | 6.14 (m, 2H), 5.67 (dd, J = 2.7, 9.6 Hz, 1H), 4.87 (br dd, J = |
| | | 5.3, 12.6 Hz, 1H), 3.69 (br dd, J = 3.7, 10.4 Hz, 1H), 3.43 |
| | | (dt, J = 6.1, 11.2 Hz, 1H), 2.98 (dt, J = 4.5, 12.1 Hz, 1H), |
| | | 2.10 (s, 3H) |
| 099 | 526.3 | δ: 8.62 (s, 1H), 7.89 (s, 1H), 7.30 (br d, J = 2.1 Hz, 2H), 7.00 |
| | | (br d, J = 8.9 Hz, 2H), 6.92 (t, J = 7.9 Hz, 1H), 6.76 (br d, J = |
| | | 8.8 Hz, 2H), 6.64 (d, J = 7.5 Hz, 1H), 6.46 (d, J = 8.1 Hz, |
| | | 1H), 6.30-6.12 (m, 2H), 5.67 (dd, J = 2.8, 9.6 Hz, 1H), 4.87 |
| | | (br dd, J = 5.1, 12.6 Hz, 1H), 3.70 (br dd, J = 3.6, 10.6 Hz, |
| | | 1H), 3.56-3.48 (m, 4H), 3.44 (td, J = 5.5, 11.2 Hz, 1H), 3.26 |
| | | (s, 3H), 3.04-2.97 (m, 1H), 2.95 (s, 3H), 2.17-1.97 (m, 3H) |
| 100 | 566.4 | δ: 8.81-8.54 (m, 1H), 8.00-7.76 (m, 2H), 7.01 (br s, 2H), |
| | | 6.92 (t, J = 7.9 Hz, 1H), 6.77 (br s, 2H), 6.64 (br d, J = 7.5 |
| | | Hz, 1H), 6.46 (d, J = 8.0 Hz, 1H), 6.30-6.14 (m, 2H), 5.68 |
| | | (dd, J = 2.9, 9.6 Hz, 1H), 4.87 (br dd, J = 5.2, 12.8 Hz, 1H), |
| | | 3.71 (br dd, J = 3.5, 10.4 Hz, 1H), 3.58-3.38 (m, 5H), 3.24 |
| | | (s, 3H), 3.06-2.69 (m, 5H), 2.14-1.99 (m, 3H), 0.72-0.31 |
| | | (m, 4H) |
| 101 | 539.4 | δ: 9.65-9.43 (m, 1H), 8.61 (s, 1H), 7.92 (s, 1H), 7.35-7.02 |
| | | (m, 4H), 6.98-6.78 (m, 3H), 6.72-6.56 (m, 1H), 6.46 (d, J = |
| | | 8.4 Hz, 1H), 6.36-6.11 (m, 2H), 5.84-5.64 (m, 1H), 4.95- |
| | | 4.83 (m, 1H), 3.69-3.67 (m, 3H), 3.49-3.36 (m, 3H), 3.29- |
| | | 3.21 (m, 2H), 3.06-2.93 (m, 4H), 2.86 (d, J = 4.8 Hz, 6H), |
| | | 2.11 (s, 3H) |
| 102 | 593.4 | δ: 9.57 (br s, 1H), 8.65-8.57 (m, 1H), 8.08-7.60 (m, 2H), |
| | | 7.07 (br s, 2H), 6.94-6.81 (m, 3H), 6.62 (br d, J = 7.5 Hz, |
| | | 1H), 6.45 (br d, J = 8.1 Hz, 1H), 6.30-6.13 (m, 2H), 5.67 (br |
| | | dd, J = 2.3, 9.8 Hz, 1H), 4.87 (br dd, J = 4.9, 12.6 Hz, 1H), |
| | | 4.55-4.37 (m, 1H), 3.69 (br d, J = 5.6 Hz, 3H), 3.47-3.39 |
| | | (m, 1H), 3.23 (br d, J = 4.1 Hz, 2H), 2.97 (br s, 1H), 2.95 (s, |
| | | 3H), 2.85 (br s, 6H), 2.10 (s, 3H), 2.05-1.80 (m, 4H), 1.63- |
| | | 1.33 (m, 2H) |
| 103 | 551.4 | δ: 8.61 (s, 1H), 7.89 (s, 1H), 7.44 (d, J = 8.4 Hz, 2H), 7.25 |
| | | (d, J = 8.4 Hz, 2H), 6.94-6.86 (m, 1H), 6.62 (br d, J = 7.4 |
| | | Hz, 1H), 6.46 (br d, J = 8.1 Hz, 1H), 6.28-6.18 (m, 2H), |
| | | 5.72-5.60 (m, 1H), 5.01-4.76 (m, 1H), 3.79-3.60 (m, 6H), |
| | | 3.58-3.44 (m, 2H), 3.31-3.09 (m, 5H), 2.78 (s, 3H), 2.11 |
| | | (s, 3H) |
| 104 | 544.4 | δ: 9.78-9.62 (m, 1H), 8.62 (s, 1H), 7.93 (s, 1H), 7.39-7.29 |
| | | (m, 2H), 7.21-7.01 (m, 3H), 6.91 (t, J = 7.9 Hz, 1H), 6.63 |
| | | (d, J = 7.4 Hz, 1H), 6.49 (d, J = 8.3 Hz, 1H), 6.33-6.10 (m, |
| | | 2H), 5.68 (dd, J = 2.6, 9.8 Hz, 1H), 4.87 (br dd, J = 4.8, 12.3 |
| | | Hz, 1H), 4.44 (br t, J = 4.7 Hz, 2H), 3.69 (br dd, J = 3.4, 10.6 |
| | | Hz, 1H), 3.59 (br d, J = 4.5 Hz, 2H), 3.43 (dt, J = 5.6, 11.2 |
| | | Hz, 1H), 2.97 (dt, J = 4.6, 12.3 Hz, 1H), 2.90 (d, J = 4.4 Hz, |
| | | 6H), 2.10 (s, 3H) |
| 105 | 500.4 | δ: 9.26-9.11 (m, 1H), 8.59 (br s, 1H), 8.15-8.04 (m, 1H), |
| | | 7.91-7.79 (m, 1H), 7.03-6.76 (m, 1H), 6.68-6.59 (m, 1H), |
| | | 6.39-6.15 (m, 3H), 5.77-5.68 (m, 1H), 5.67-5.47 (m, 1H), |
| | | 4.95-4.83 (m, 1H), 3.73-3.61 (m, 1H), 3.54 (br d, J = 7.0 |
| | | Hz, 2H), 3.48-3.35 (m, 1H), 3.26-2.95 (m, 5H), 2.90 (br s, |
| | | 1H), 2.80 (br d, J = 4.5 Hz, 3H), 2.16-2.01 (m, 3H), 1.91- |
| | | 1.80 (m, 2H), 0.91-0.74 (m, 2H), 0.62-0.46 (m, 2H) |
| 106 | 487.3 | δ: 8.59 (br s, 1H), 8.14-8.04 (m, 1H), 7.87 (s, 1H), 6.89 (t, J = |
| | | 7.8 Hz, 1H), 6.63 (d, J = 7.5 Hz, 1H), 6.33-6.24 (m, 3H), |
| | | 5.73 (dd, J = 3.6, 8.8 Hz, 1H), 4.87 (br dd, J = 4.9, 12.8 Hz, |
| | | 1H), 4.45-4.21 (m, 2H), 3.82-3.73 (m, 1H), 3.70-3.59 (m, |
| | | 3H), 3.57-3.48 (m, 1H), 3.46-3.37 (m, 1H), 2.99 (dt, J = |
| | | 4.8, 12.2 Hz, 1H), 2.79 (br s, 2H), 2.12 (s, 3H), 1.93-1.82 |
| | | (m, 1H), 1.74-1.61 (m, 1H), 0.77-0.71 (m, 2H), 0.58-0.52 |
| | | (m, 2H) |

### Example 2: HTRF-based EGFR biochemical assays

To measure the IC₅₀ values of compounds herein against EGFR, a Z′-LYTE assay (ThermoFisher) is used. Briefly, 2.5 μL of different concentrations of the compounds in 4% DMSO are added to 5 μL kinase/peptide mixture in each well of a 384-well plate (Corning Cat. #3676). 2.5 μL of 4× ATP solution (4× ATP, 50 mM HEPES, pH 7.5, 0.01% BRIJ-35, 10 mM MgCl₂, 1 mM EGTA) are added to each well. The plate is shaken for 30 seconds, and then incubated at room temperature for 60 minutes. 5 μL of a 1:100000 dilution of Development Reagent A is added to each well. The plate is then incubated for 60 mins at room temperature. 5 μL of the stop reagent is then added. The plate is subsequently read on a fluorescence plate reader, and the emissions ratio is calculated to determine the ratio of Ser/Thr 07 phosphorylated by the reaction. Emissions Ratio=Coumarin Emission (443 nm)/Flourescein Emission (520 nm).

### Example 3: Ba/F3 Cell Proliferation Models

The EGFR mutant L858R Ba/F3 cells have been previously described (Zhou, W., et al. Nature 462, 2009, 1070-1074). The EGFR C797S and C775S mutations were introduced via site directed mutagenesis using the Quick Change Site-Directed Mutagenesis kit into a vector containing EGFR L858R mutation (Stratagene; La Jolla, CA) according to the manufacturer's instructions. All constructs were confirmed by DNA sequencing. The constructs were then shuttled into the retroviral vector JP1540 by either using the Cre-recombination system (Agilent Technologies, Santa Clara, CA) or the In-fusion HD Cloning kit (Takara Bio USA, Inc.; Mountain view, CA). Ba/F3 cells were then infected with retrovirus per standard protocols, as described previously (Zhou, et al, Nature 2009). Stable clones were obtained by selection in puromycin (2 ug/ml). All BaF/3 mutant cells were maintained in RPMI 1640 (Cellgro; Mediatech Inc., Herndon, CA) supplemented with 10% FBS, 100 units/mL penicillin, 100 units/mL streptomycin.

Growth and inhibition of growth was assessed by the Cell Titer Glo assay (Promega, Madison, WI) and was performed according to the manufacturer's instructions. The Cell Titer Glo assay is a luminescence-based method used to determine the number of viable cells based on quantitation of the ATP present, which is directly proportional to the amount of metabolically active cells present. Ba/F3 cells of different EGFR genotypes were exposed to compounds for 72 hours and the number of cells used per experiment was determined empirically as has been previously established (Zhou, et al., Nature 2009). All experimental points were set up in triplicates in 384-well plates. The luminescent signal was detected using a spectrometer and the data was graphically displayed using GraphPad Prism version 5.0 for Windows, (GraphPad Software; www.graphpad.com). The curves were fitted using a non-linear regression model with a sigmoidal dose response.

Ba/F3 cells assay data of the test compounds are provided in Table 4 below. For inhibitory activity against EGFR mutants, the following designations are used: ≤0·25 μM=A, 0·25 μM˜0.5 μM=B, 0.5 μM˜2.5 μM=C, ≥2.5 μM=D.

TABLE 4

| Compound | L858R BaF3 | L858R/C797S | L858R/C775S |
|---|---|---|---|
| No. | (μM) | (μM) | (μM) |
| 001 | B | C | C |
| 002 | A | B | D |
| 003 | C | D | C |
| 004 | B | C | D |
| 005 | C | D | D |
| 006 | B | D | D |
| 007 | B | D | D |
| 008 | C | D | D |
| 009 | D | D | D |
| 010 | A | A | D |
| 011 | D | D | N/D |
| 012 | D | D | N/D |
| 013 | A | B | N/D |
| 014 | A | A | N/D |
| 015 | D | D | N/D |
| 016 | D | D | N/D |
| 017 | A | A | N/D |
| 018 | D | D | N/D |
| 019 | A | A | N/D |
| 020 | A | B | N/D |
| 021 | A | A | N/D |
| 022 | D | D | N/D |
| 023 | D | D | N/D |
| 024 | D | D | N/D |
| 025 | D | D | N/D |
| 026 | C | C | N/D |
| 027 | A | A | N/D |
| 028 | A | B | N/D |
| 029 | A | A | N/D |
| 030 | A | A | N/D |
| 031 | A | A | N/D |
| 032 | A | A | N/D |
| 033 | A | A | N/D |
| 034 | A | A | N/D |
| 035 | A | A | N/D |
| 036 | A | A | N/D |
| 037 | A | A | N/D |
| 038 | A | A | N/D |
| 039 | A | A | N/D |
| 040 | A | A | N/D |
| 041 | A | B | N/D |
| 042 | A | A | N/D |
| 043 | A | A | N/D |
| 044 | A | A | N/D |
| 045 | A | A | N/D |
| 046 | A | A | N/D |
| 047 | A | A | N/D |
| 048 | A | A | N/D |
| 049 | A | A | N/D |
| 050 | A | A | N/D |
| 051 | A | A | N/D |
| 052 | A | A | N/D |
| 053 | A | B | N/D |
| 054 | A | A | N/D |
| 055 | A | A | N/D |
| 056 | A | A | N/D |
| 057 | A | A | N/D |
| 058 | A | A | N/D |
| 059 | A | A | N/D |
| 060 | B | C | N/D |
| 061 | A | A | N/D |
| 062 | A | A | N/D |
| 063 | B | C | N/D |
| 064 | A | B | N/D |
| 065 | A | A | N/D |
| 066 | A | A | N/D |
| 067 | A | A | N/D |
| 068 | A | A | N/D |
| 069 | A | A | N/D |
| 070 | A | A | N/D |
| 071 | A | A | N/D |
| 072 | A | A | N/D |
| 073 | A | A | N/D |
| 074 | A | A | N/D |
| 075 | A | A | N/D |
| 076 | A | A | N/D |
| 077 | A | A | N/D |
| 078 | A | A | N/D |
| 079 | A | C | N/D |
| 080 | A | A | N/D |
| 081 | A | A | N/D |
| 082 | A | A | N/D |
| 083 | C | C | N/D |
| 084 | A | A | N/D |
| 085 | D | D | N/D |
| 086 | A | A | N/D |
| 087 | A | A | N/D |
| 088 | A | A | N/D |
| 089 | A | A | N/D |
| 090 | A | B | N/D |
| 091 | A | B | N/D |
| 092 | A | A | N/D |
| 093 | A | B | N/D |
| 094 | A | A | N/D |
| 095 | A | A | N/D |
| 096 | A | A | N/D |
| 097 | A | A | N/D |
| 098 | B | C | N/D |
| 099 | A | A | N/D |
| 100 | A | A | N/D |
| 101 | B | C | N/D |
| 102 | A | A | N/D |
| 103 | B | C | N/D |
| 104 | A | A | N/D |
| 105 | A | A | N/D |
| 106 | A | B | N/D |

*N/D = not determined

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula A-I: 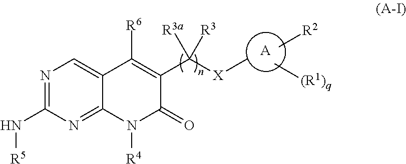
or a pharmaceutically acceptable salt thereof;
wherein:
X is O, S, NH, CH₂, or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-11 membered bicyclic ring;
each R¹ is independently selected from the group consisting of H, ═O, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, NS(O)(C₁-C₆ alkyl)₂, NS(NH)(C₁-C₆ alkyl)₂, NS(NC₁-C₆ alkyl) (C₁-C₆ alkyl)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
q is 1, 2, or 3;
R³ and R^{3a} are each independently selected from the group consisting of H, halo, CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with R⁹;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted with one or two R⁸;
R⁵ is selected from the group consisting of H, C₁-C₆ alkyl, C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl, 6-11 membered bicyclic ring, wherein 3-10 membered heterocycloalkyl, 6-11 membered bicyclic ring, C₆-C₁₀ aryl, and 5-10 membered heteroaryl are each optionally substituted with one, two, or three R^{5a};
each R^{5a} is independently selected from the group consisting of ═O, 3-10 membered heterocycloalkyl, O-3-10 membered heterocycloalkyl, halo, NS(O)(C₁-C₆ alkyl)₂, NS(NH)(C₁-C₆ alkyl)₂, NS(NC₁-C₆ alkyl) (C₁-C₆ alkyl)₂, SO₂C₁-C₆ alkyl, C₁-C₆ alkyl, C₁-C₆ alkoxy, and C₃-C₁₀ cycloalkyl, wherein 3-10 membered heterocycloalkyl, O-3-10 membered heterocycloalkyl, C₁-C₆ alkyl, and C₁-C₆ alkoxy are optionally substituted with one, two, or three R^{5aa};
each R^{5aa} is independently selected from the group consisting of ═O, C₁-C₆ alkyl, C₁-C₆ haloalkyl, halo, CN, OH, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-10 membered heterocycloalkyl, wherein 3-10 membered heterocycloalkyl is optionally substituted with one or two C₁-C₆ alkyl or SO₂C₁-C₆ alkyl;
R⁶ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
each R⁸ is independently selected from halo, C₁-C₆ alkyl, C₁-C₆ alkoxy, C₁-C₆ haloalkoxy, C₃-C₁₀ cycloalkyl, 3-10 membered heterocycloalkyl, O-3-10 membered heterocycloalkyl, C₆-C₁₀ aryl, 5-10 membered heteroaryl, OH, CN, NH₂, NH(C₁-C₆ alkyl), N(C₁-C₆ alkyl)₂, C₁-C₆ alkyl-OH, C(O)C₁-C₆ alkyl-OH, SO₂C₁-C₆ alkyl, SO₂C₃-C₆ cycloalkyl, C(O)C₁-C₆ alkyl, C(O)NHC₁-C₆ alkyl, NHC(O)C₁-C₆ alkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl), wherein C₁-C₆ alkyl, C₁-C₆ alkoxy, 3-10 membered heterocycloalkyl, O-3-10 membered heterocycloalkyl, NH(C₁-C₆ alkyl), and N(C₁-C₆ alkyl)₂ are optionally substituted with one or two R^{8a};
R^{8a} is selected from C₁-C₆ alkyl, C₁-C₆ alkoxy, OH, NH₂, NH(C₁-C₆ alkyl), N(C₁-C₆ alkyl)₂, and 3-10 membered heterocycloalkyl, wherein 3-10 membered heterocycloalkyl is optionally substituted with one or two C₁-C₆ alkyl and C₁-C₆ alkoxy;
each R⁹ is independently selected from the group consisting of halo, CN, OH, C₁-C₆ alkyl, OC₁-C₆ alkoxy, NH₂, NH(C₁-C₆ alkyl), and N(C₁-C₆ alkyl)₂;
R² is selected from the group consisting of: 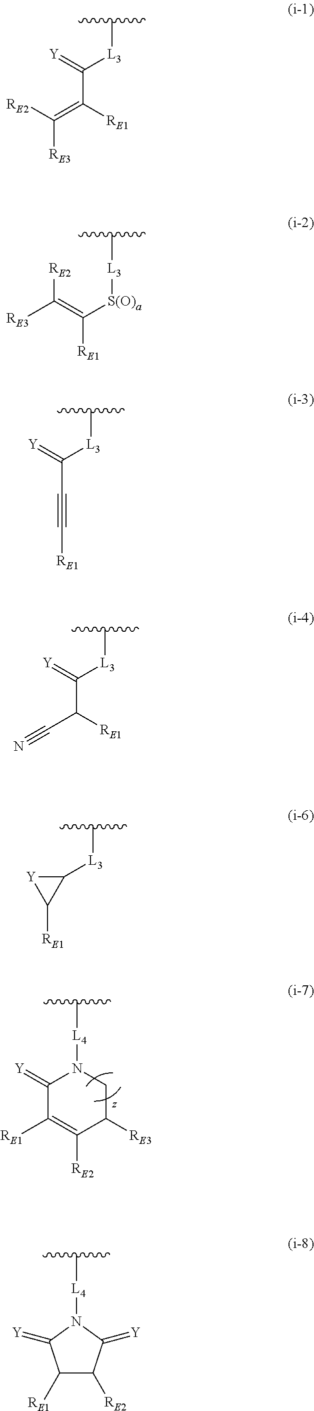 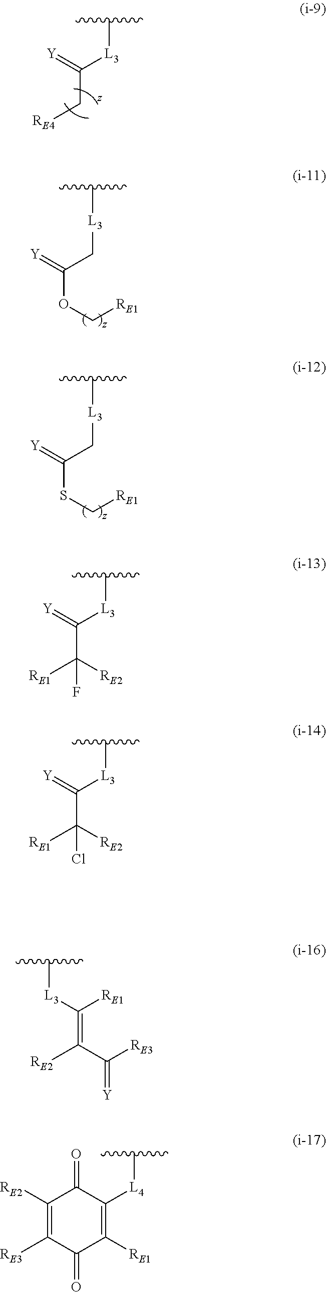 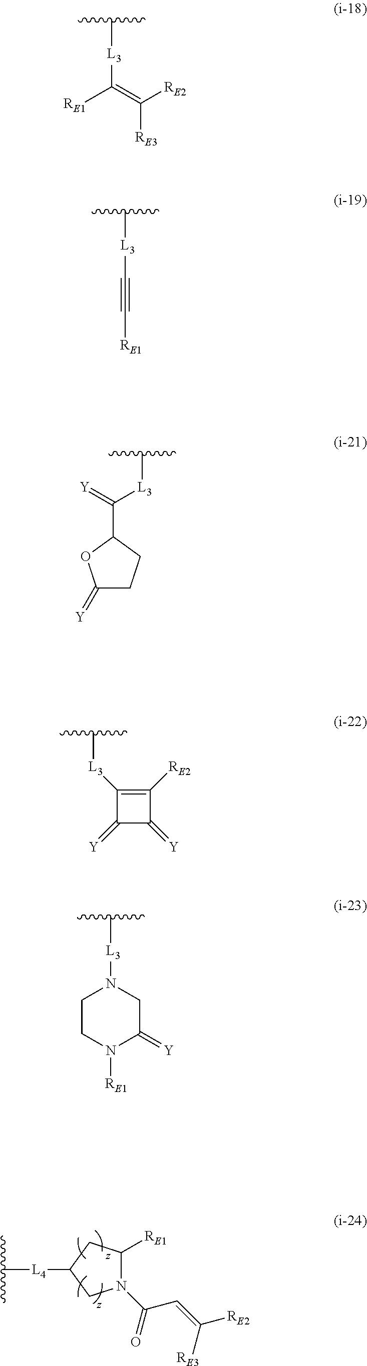 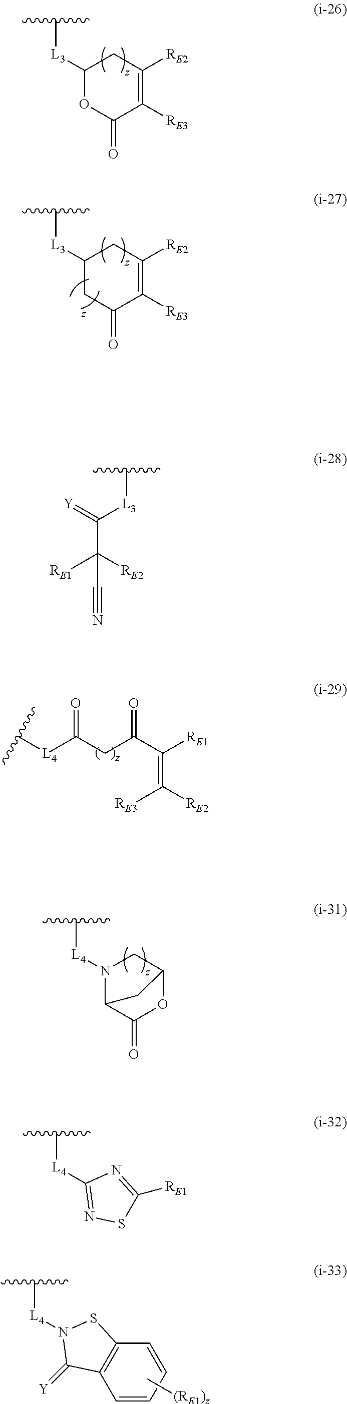 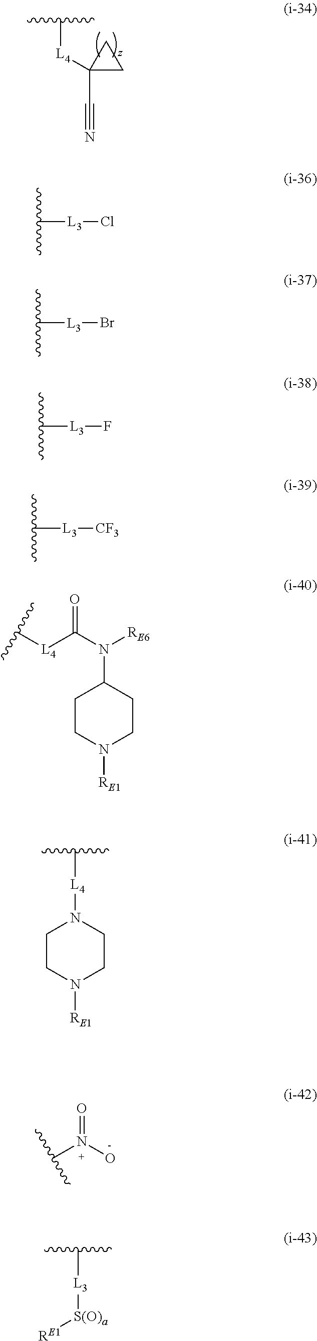 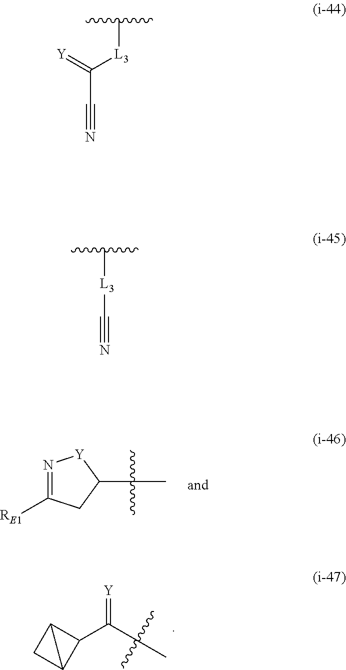
L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)-, —C(S) NR_{L3a}—, trans —CR_{L3b}═CR_{L3b}—, cis —CR_{L3b}═CR_{L3b}—, —C═C-, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R^{EE})₂, CH₂SR_{EE}, OR_{EE}, N(R^{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

2. The compound of claim 1, wherein
X is O, S, NH, CH₂ or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
each R¹ is independently selected from the group consisting of H, ═O, halo, CN, OR⁷, C₁-C₆ alkyl, C₁-C₆ haloalkyl, N(R⁷)₂, C₆-C₁₀ aryl, and 5-10 membered heteroaryl;
q is 1, 2, or 3;
R³ and R^{3a} are each independently selected from the group consisting of H, halo, and C₁-C₆ alkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, heterocycloalkyl, and heteroaryl are each optionally substituted with one R⁸, and wherein cycloalkyl and aryl are optionally substituted with one or two R⁸;
R⁵ is selected from the group consisting of H, C₁-C₆ alkyl, C₆-C₁₀ aryl, 5-10 membered heteroaryl, C₃-C₁₀ cycloalkyl, 3-10 membered heterocycloalkyl, and 6-11 membered bicyclic ring, wherein C₆-C₁₀ aryl, 5-10 membered heteroaryl, and 6-11 membered bicyclic ring are each optionally substituted with one, two, or three R^{5a};
each R^{5a} is independently selected from ═O, 3-10 membered heterocycloalkyl, O-3-10 membered heterocycloalkyl, halo, SO₂C₁-C₆ alkyl, C₁-C₆ alkyl, and C₁-C₆ alkoxy, wherein O-3-membered heterocycloalkyl, C₁-C₆ alkyl, and C₁-C₆ alkoxy are optionally substituted with one R^{5aa} and 3-10 membered heterocycloalkyl is optionally substituted with one or two R^{5aa};
each R^{5aa} is independently selected from ═O, C₁-C₆ alkyl, C₁-C₆ haloalkyl, halo, CN, OH, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-10 membered heterocycloalkyl, wherein 3-10 membered heterocycloalkyl is optionally substituted with one C₁-C₆ alkyl or SO₂C₁-C₆ alkyl;
R⁶ is selected from the group consisting of H, halo, OH, CN, C₁-C₆ alkyl, and C₁-C₆ haloalkyl;
each R⁷ is independently H or C₁-C₆ alkyl;
each R⁸ is independently selected from OH, halo, C₁-C₆ alkyl, C₁-C₆ alkoxy, C₁-C₆ haloalkoxy, 3-10 membered heterocycloalkyl, O-3-10 membered heterocycloalkyl, NH₂, NH(C₁-C₆ alkyl), N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-10 membered heterocycloalkyl, O-3-10 membered heterocycloalkyl, NH(C₁-C₆ alkyl), and N(C₁-C₆ alkyl)₂ are optionally substituted with one or two R^{8a};
R^{8a} is selected from C₁-C₆ alkyl, C₁-C₆ alkoxy, OH, NH₂, NH(C₁-C₆ alkyl), N(C₁-C₆ alkyl)₂, and 3-10 membered heterocycloalkyl, wherein 3-10 membered heterocycloalkyl is optionally substituted with one C₁-C₆ alkyl or C₁-C₆ alkoxy;
R² is selected from the group consisting of: 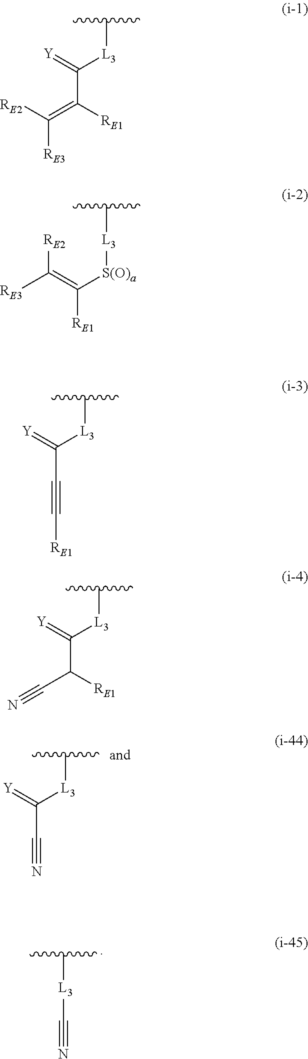
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl;
each Y is independently O, S, or CH₂; and
a is 0, 1, or 2.

3. The compound of claim 1 or claim 2, wherein
X is O, S, or a bond;
A is selected from the group consisting of C₆-C₁₀ aryl, 5-10 membered heteroaryl, 3-10 membered heterocycloalkyl, and 6-10 membered bicyclic ring;
each R¹ is independently selected from the group consisting of H, ═O, halo, CN, OH, C₁-C₆ alkyl, C₁-C₆ alkoxy, C₁-C₆ haloalkyl, NH₂, and phenyl;
q is 1, 2, or 3;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₆ cycloalkyl, 3-6 membered heterocycloalkyl, C₆-C₈ aryl, and 6-8 membered heteroaryl, wherein alkyl, heterocycloalkyl, and heteroaryl are each optionally substituted with one R⁸, and wherein cycloalkyl and aryl are optionally substituted with one or two R⁸;
R⁵ is selected from the group consisting of H, C₁-C₆ alkyl, C₆-C₈ aryl, 5-7 membered heteroaryl, C₃-C₆ cycloalkyl, 3-6 membered heterocycloalkyl, and 8-11 membered bicyclic ring, wherein C₆-C₁₀ aryl, 5-7 membered heteroaryl, and 8-11 membered bicyclic ring are each optionally substituted with one or two R^{5a};
each R^{5a} is independently selected from ═O, 3-6 membered heterocycloalkyl, O-3-6 membered heterocycloalkyl, halo, SO₂C₁-C₆ alkyl, C₁-C₆ alkyl, and C₁-C₆ alkoxy, wherein 3-6 membered heterocycloalkyl, O-3-6 membered heterocycloalkyl, C₁-C₆ alkyl, and C₁-C₆ alkoxy are optionally substituted with one R^{5aa} and 3-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, halo, and CN;
each R⁸ is independently selected from OH, halo, C₁-C₆ alkyl, C₁-C₆ alkoxy, C₁-C₆ haloalkoxy, 3-6 membered heterocycloalkyl, O-3-6 membered heterocycloalkyl, NH₂, NH(C₁-C₆ alkyl), N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-6 membered heterocycloalkyl, O-3-6 membered heterocycloalkyl, NH(C₁-C₆ alkyl), and N(C₁-C₆ alkyl)₂ are optionally substituted with one R^{8a};
R^{8a} is selected from C₁-C₆ alkyl, C₁-C₆ alkoxy, OH, NH₂, NH(C₁-C₆ alkyl), N(C₁-C₆ alkyl)₂, and 3-6 membered heterocycloalkyl, wherein 3-6 membered heterocycloalkyl is optionally substituted with one C₁-C₆ alkyl or C₁-C₆ alkoxy;
R² is selected from the group consisting of: 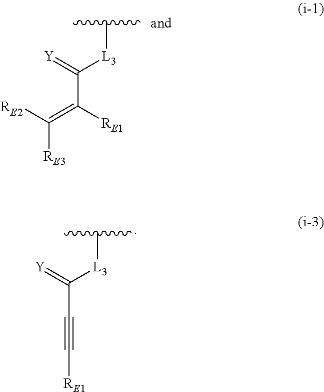
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

4. The compound of any one of claims 1-3, wherein
X is O, S, or a bond;
A is selected from the group consisting of phenyl, indole, piperazine, indoline, piperidine, tetrahydroquinoxaline, quinoxaline, tetrahydroquinoline, quinoline, azaspiro[2.5]octane, azaspiro[3.5]nonane, azaspiro[4.5]decane, diazaspiro[2.5]octane, diazaspiro[3.5]nonane, diazaspiro[4.5]decane, 2,5-diazabicyclo[4.1.0]heptane, diazabicyclo[4·2.0]octane, octahydro-1H-cyclopenta[c]pyridine, octahydro-1H-cyclopenta[b]pyrazine, and octahydro-1H-pyrrolo[3,4-c]pyridine;
each R¹ is independently selected from the group consisting of H, C₁-C₃ alkyl, and C₁-C₃ haloalkyl;
q is 1, 2, or 3;
R³ and R^{3a} are each independently selected from the group consisting of H, halo, and C₁-C₃ alkyl;
n is 0 or 1;
R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸;
R⁵ is selected from the group consisting of H, C₁-C₆ alkyl, C₆-C₁₀ aryl, 5-10 membered heteroaryl, C₃-C₆ cycloalkyl, 9-10 membered bicyclic ring, and 4-5 membered heterocycloalkyl, wherein C₆-C₁₀ aryl, 5-10 membered heteroaryl, and 9-10 membered bicyclic ring are each optionally substituted with one or two R^{5a};
each R^{5a} is independently selected from ═O, 3-6 membered heterocycloalkyl, O-3-6 membered heterocycloalkyl, halo, SO₂C₁-C₆ alkyl, C₁-C₆ alkyl, and C₁-C₆ alkoxy, wherein 3-6 membered heterocycloalkyl, O-3-6 membered heterocycloalkyl, C₁-C₆ alkyl, and C₁-C₆ alkoxy are optionally substituted with one R^{5aa} and 3-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, C₁-C₃ alkyl, and C₁-C₃ haloalkyl;
each R⁸ is independently selected from OH, halo, C₁-C₆ alkyl, C₁-C₆ alkoxy, C₁-C₆ haloalkoxy, 3-6 membered heterocycloalkyl, O-3-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 3-6 membered heterocycloalkyl, and N(C₁-C₆ alkyl)₂ are optionally substituted with one R^{8a};
R^{8a} is selected from C₁-C₆ alkyl, C₁-C₆ alkoxy, OH, NH₂, NH(C₁-C₆ alkyl), N(C₁-C₆ alkyl)₂, and 3-6 membered heterocycloalkyl, wherein 3-6 membered heterocycloalkyl is optionally substituted with one C₁-C₆ alkyl or C₁-C₆ alkoxy;
R² is selected from the group consisting of: 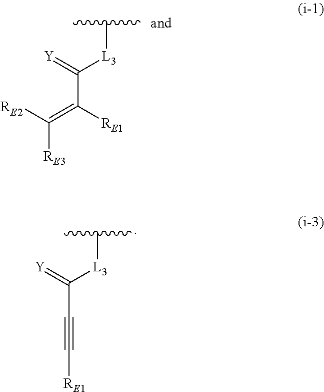
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

5. The compound of any one of claims 1-4, wherein
X is O, S, or a bond;
A is selected from the group consisting of: 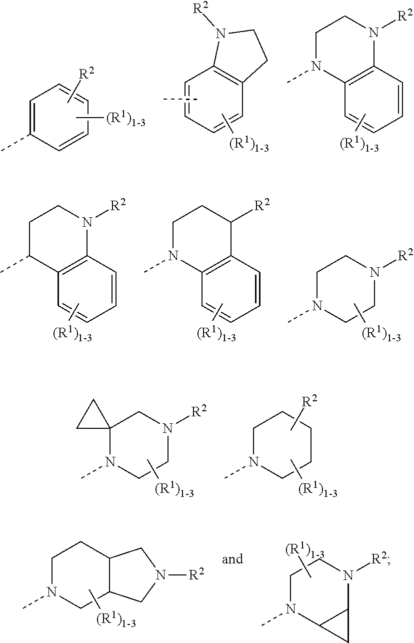
each R¹ is independently selected from the group consisting of H, ═O, halo, OH, C₁-C₆ alkyl, C₁-C₆ alkoxy, NH₂, and phenyl;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₆ cycloalkyl, 4-6 membered heterocycloalkyl, phenyl, and 6-membered heteroaryl, wherein alkyl, heterocycloalkyl, and heteroaryl are each optionally substituted with one R⁸, and wherein cycloalkyl and phenyl are optionally substituted with one or two R⁸;
R⁵ is selected from the group consisting of H, C₁-C₆ alkyl, phenyl, 5-6 membered heteroaryl, C₃-C₆ cycloalkyl, 9-10 membered bicyclic ring, and 4-5 membered heterocycloalkyl, wherein phenyl, 5-6 membered heteroaryl, and 9-10 membered bicyclic ring are each optionally substituted with one or two R^{5a};
each R^{5a} is independently selected from ═O, 5-6 membered heterocycloalkyl, O-3-4 membered heterocycloalkyl, halo, SO₂C₁-C₆ alkyl, C₁-C₆ alkyl, and C₁-C₆ alkoxy, wherein 5-6 membered heterocycloalkyl, O-3-4 membered heterocycloalkyl, C₁-C₆ alkyl, and C₁-C₆ alkoxy are optionally substituted with one R^{5aa} and 5-6 membered heterocycloalkyl is optionally substituted with one or two R^{5aa};
each R^{5aa} is independently selected from ═O, C₁-C₆ alkyl, C₁-C₆ haloalkyl, halo, CN, OH, C₁-C₆ alkyl, SO₂C₁-C₆ alkyl, NH₂, N(H)(C₁-C₆ alkyl), N(C₁-C₆ alkyl)₂, SO₂NH₂, SO₂N(H)(C₁-C₆ alkyl), SO₂N(C₁-C₆ alkyl)₂, and 3-6 membered heterocycloalkyl, wherein 3-6 membered heterocycloalkyl is optionally substituted with one C₁-C₆ alkyl or SO₂C₁-C₆ alkyl;
R⁶ is selected from the group consisting of H, C₁-C₃ alkyl, and C₁-C₃ haloalkyl;
each R⁸ is independently selected from OH, halo, C₁-C₆ alkyl, C₁-C₆ alkoxy, C₁-C₆ haloalkoxy, 4-6 membered heterocycloalkyl, O-4-6 membered heterocycloalkyl, N(C₁-C₆ alkyl)₂, C(O)C₁-C₆ alkyl-OH, C(O)C₁-C₆ alkyl, C(O)NHC₁-C₆ alkyl, and NHC(O)C₁-C₆ alkyl, wherein C₁-C₆ alkyl, C₁-C₆ alkoxy, 4-6 membered heterocycloalkyl, and N(C₁-C₆ alkyl)₂ are optionally substituted with one R^{8a};
R^{8a} is selected from C₁-C₆ alkyl, C₁-C₆ alkoxy, OH, NH₂, NH(C₁-C₆ alkyl), N(C₁-C₆ alkyl)₂, and 5-6 membered heterocycloalkyl, wherein 5-6 membered heterocycloalkyl is optionally substituted with one C₁-C₆ alkyl or C₁-C₆ alkoxy;
R² is selected from the group consisting of: 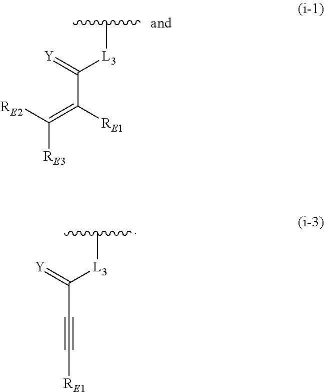
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

6. The compound of claim 1, wherein the compound is a compound of Formula I: 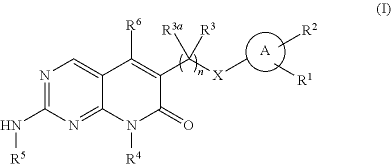
or a pharmaceutically acceptable salt thereof;
wherein:
X is O, S, NH, CH₂, or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-11 membered bicyclic ring;
R¹ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
R³ and R^{3a} are each independently selected from the group consisting of H, halo, CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with Ro;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein alkyl, cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl, 6-11 membered bicyclic ring, wherein 3-10 membered heterocycloalkyl, 6-11 membered bicyclic ring, C₆-C₁₀ aryl, and 5-10 membered heteroaryl are each optionally substituted with one, two, or three R^{5a};
each R^{5a} is independently selected from 3-10 membered heterocycloalkyl, C₁-C₆ alkoxy, and C₃-C₁₀ cycloalkyl, wherein 3-10 membered heterocycloalkyl is optionally substituted with C₁-C₆ alkyl;
R⁶ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O) R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
R⁸ is selected from C₁-C₆ alkyl, C₃-C₁₀ cycloalkyl, 3-10 membered heterocycloalkyl, C₆-C₁₀ aryl, and 5-10 membered heteroaryl, OH, CN, NH₂, NH(C₁-C₆ alkyl), N(C₁-C₆ alkyl)₂, C₁-C₆ alkyl-OH, C(O)C₁-C₆ alkyl-OH, SO₂C₁-C₆ alkyl, SO₂C₃-C₆ cycloalkyl, halo, C(O)C₁-C₆ alkyl, C₁-C₆ alkyl, C₁-C₆ alkoxy, NHC(O)C₁-C₆ alkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl);
each R⁹ is independently selected from the group consisting of halo, CN, OH, C₁-C₆ alkyl, OC₁-C₆ alkoxy, NH₂, NH(C₁-C₆ alkyl), and N(C₁-C₆ alkyl)₂;
R² is selected from the group consisting of: 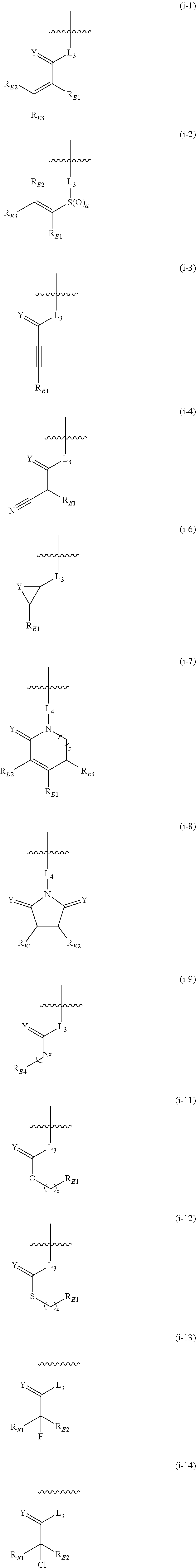 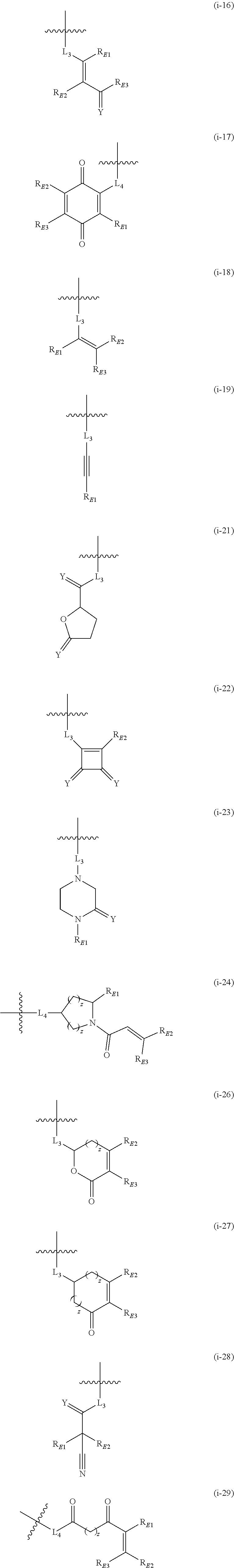 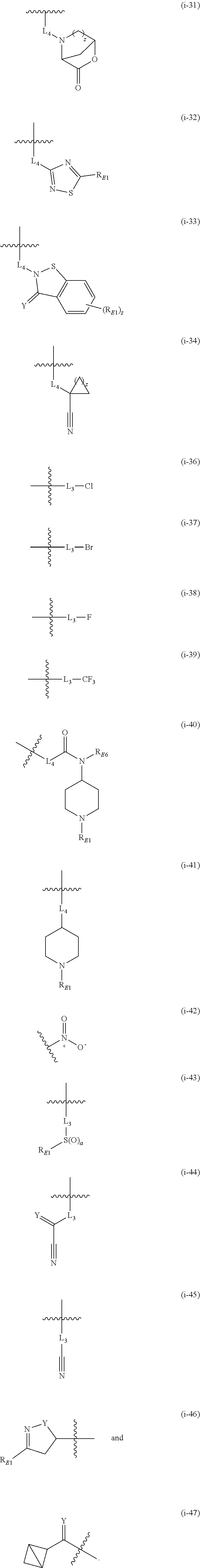
L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}-SC(O)—, —C(O)S—, —OC(O)—, —C(O)O—, —NR¹³aC(S)-, —C(S) NR_{L3a}—, trans —CR_{L3b}═CR_{L3b}—, cis —CR_{L3b}═CR_{L3b}—, —C═C-, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R^{EE})₂, CH₂SR_{EE}, OR_{EE}, N(R^{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

7. The compound of claim 6, wherein
X is O, S, NH, CH₂ or a bond;
A is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
R¹ is selected from the group consisting of H, halo, CN, C₁-C₆ alkyl, and C₁-C₆ haloalkyl;
R³ and R^{3a} are each independently selected from the group consisting of H, halo, and C₁-C₆ alkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl, wherein cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, and 3-10 membered heterocycloalkyl, wherein C₆-C₁₀ aryl and 5-10 membered heteroaryl are each optionally substituted with one, two, or three R^{5a};
each R^{5a} is independently selected from 3-10 membered heterocycloalkyl and C₁-C₆ alkoxy, wherein 3-10 membered heterocycloalkyl is optionally substituted with C₁-C₆ alkyl;
R⁶ is selected from the group consisting of H, halo, CN, C₁-C₆ alkyl, and C₁-C₆ haloalkyl;
R⁸ is selected from C(O)C₁-C₆ alkyl-OH, C(O)C₁-C₆ alkyl, and NHC(O)C₁-C₆ alkyl;
R² is selected from the group consisting of: 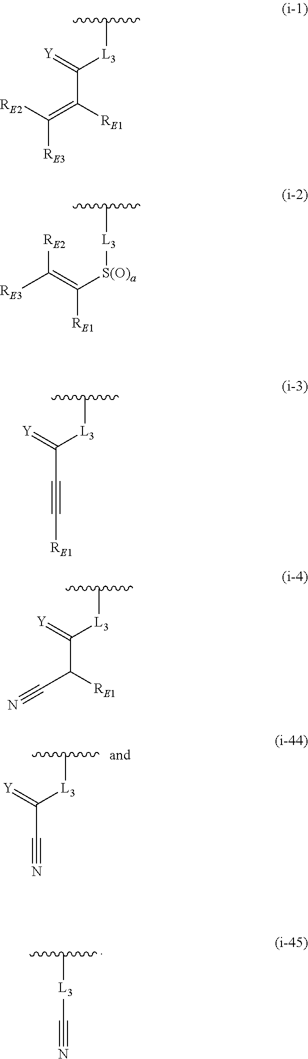
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl;
each Y is independently O, S, or CH₂; and
a is 0, 1, or 2.

8. The compound of claim 6 or claim 7, wherein
X is O, S, or a bond;
A is selected from the group consisting of C₆-C₁₀ aryl, 5-10 membered heteroaryl, 3-10 membered heterocycloalkyl, and 6-10 membered bicyclic ring;
R¹ is selected from the group consisting of H, halo, CN, C₁-C₆ alkyl, and C₁-C₆ haloalkyl;
R³ and R^{3a} are each independently selected from the group consisting of H, halo, and C₁-C₆ alkyl;
n is 0 or 1;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R⁸;
R⁵ is selected from the group consisting of C₆-C₁₀ aryl, 5-10 membered heteroaryl, C₃-C₁₀ cycloalkyl, and 3-10 membered heterocycloalkyl, wherein C₆-C₁₀ aryl and 5-10 membered heteroaryl are each optionally substituted with one, two, or three R^{5a};
each R^{5a} is independently selected from 3-10 membered heterocycloalkyl and C₁-C₆ alkoxy, wherein 3-10 membered heterocycloalkyl is optionally substituted with C₁-C₆ alkyl;
R⁶ is selected from the group consisting of H, halo, CN, C₁-C₆ alkyl, and C₁-C₆ haloalkyl;
R⁸ is selected from C(O)C₁-C₆ alkyl-OH, C(O)C₁-C₆ alkyl, and NHC(O)C₁-C₆ alkyl;
R² is selected from the group consisting of: 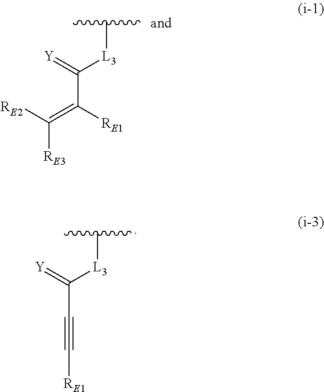
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

9. The compound of any one of claims 6-8 wherein
X is O, S, or a bond;
A is selected from the group consisting of phenyl, indole, piperazine, indoline, piperidine, and tetrahydroquinoxaline;
R¹ is selected from the group consisting of H, C₁-C₃ alkyl, and C₁-C₃ haloalkyl;
R³ and R^{3a} are each independently selected from the group consisting of H, halo, and C₁-C₃ alkyl;
n is 0 or 1;
R⁴ is selected from the group consisting of H, C₁-C₃ alkyl, 6-10 membered heterocycloalkyl, and C₆-C₁₀ aryl; wherein heterocycloalkyl and aryl are each optionally substituted with R⁸;
R⁵ is selected from the group consisting of C₆-C₁₀ aryl and 5-10 membered heteroaryl, wherein C₆-C₁₀ aryl and 5-10 membered heteroaryl are each optionally substituted with one or two R^{5a};
each R^{5a} is independently selected from 6-10 membered heterocycloalkyl and C₁-C₃ alkoxy, wherein the 6-10 membered heterocycloalkyl is optionally substituted with C₁-C₃ alkyl;
R⁶ is selected from the group consisting of H, C₁-C₃ alkyl, and C₁-C₃ haloalkyl;
R⁸ is selected from C(O)C₁-C₃ alkyl-OH, C(O)C₁-C₃ alkyl, and NHC(O)C₁-C₃ alkyl;
R² is selected from the group consisting of: 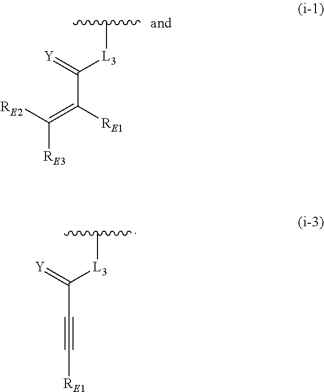
L₃ is a bond, —NH—, or —N(C₁-C₄ alkyl)-;
each of R_{E1}, R_{E2}, and R_{E3} is independently selected from the group consisting of hydrogen, halogen, and C₁-C₆ alkyl; and
each Y is independently O, S, or CH₂.

10. The compound of any one of claims 1-9, wherein the compound of Formula I is a compound of Formula IA: 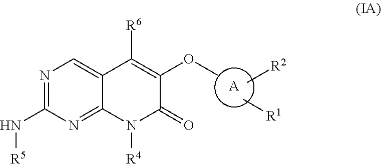
or a pharmaceutically acceptable salt thereof.

11. The compound of any one of claims 1-9, wherein the compound of Formula I is a compound of Formula IB: 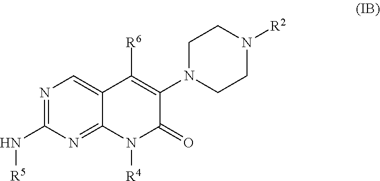
or a pharmaceutically acceptable salt thereof.

12. The compound of any one of claims 1-9, wherein R³ and R^{3a} are each independently H or C₁-C₃ alkyl.

13. The compound of any one of claims 1-9 and 12, wherein X is O or a bond.

14. The compound of any one of claims 1-9, 12, and 13, wherein X is O.

15. The compound of any one of claims 1-9, 12, and 13, wherein X is a bond.

16. The compound of claim 1 or claim 6, wherein R³ and R^{3a} are each independently CN, C₁-C₆ alkyl, C₁-C₆ alkoxy, NH₂, NH(C₁-C₆ alkyl), and N(C₁-C₆ alkyl)₂, wherein C₁-C₆ alkyl is optionally substituted one, two, or three times with R⁹.

17. The compound of any one of claims 1, 6, and 16, wherein R³ and R^{3a} are each independently CN, C₁-C₃ alkyl, C₁-C₃ alkoxy, NH(C₁-C₃ alkyl), and N(C₁-C₃ alkyl)₂, wherein C₁-C₃ alkyl is optionally substituted one or two times with R⁹.

18. The compound of any one of claims 1, 6, 16, and 17, wherein R³ and R^{3a} are each independently CN, CH₂CN, OCH₃, NH(CH₃), and N(CH₃)₂.

19. The compound of any one of claims 1 6, and 16-18, wherein R⁵ is 3-10 membered heterocycloalkyl or 6-11 membered bicyclic ring, wherein 3-10 membered heterocycloalkyl and 6-11 membered bicyclic ring are optionally substituted with R^{5a}.

20. The compound of any one of claims 1, 6 and 16-19, wherein R^{5a} is C₃-C₆ cycloalkyl and wherein R⁵ is substituted by cycloalkyl in spiro configuration.

21. The compound of any one of claims 1-4, 6-10, and 12-20, wherein A is piperazine or tetrahydroquinoxaline.

22. The compound of any one of claims 1-10 and 12-21, wherein R¹ is H or C₁-C₃ alkyl.

23. The compound of any one of claims 1-22, wherein R⁴ is H, C₁-C₃ alkyl, piperidine, and phenyl, wherein piperidine and phenyl are optionally substituted with R⁸.

24. The compound of any one of claims 1-4 and 6-23, wherein R⁵ is phenyl and wherein each R^{5a} is independently 6-10 membered heterocycloalkyl or C₁-C₃ alkoxy, wherein 6-10 membered heterocycloalkyl is optionally substituted with C₁-C₃ alkyl.

25. The compound of any one of claims 1-9 and 12-24, wherein the compound of Formula I is a compound of Formula IC: 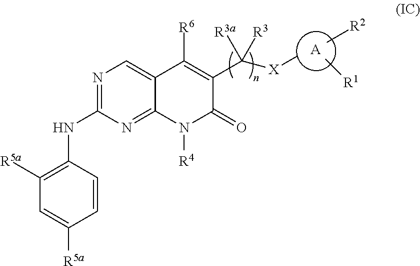
or a pharmaceutically acceptable salt thereof.

26. The compound of any one of claims 1-19 and 21-25, wherein each R^{5a} is individually selected from piperazine and C₁-C₃ alkoxy, wherein piperazine is optionally substituted with C₁-C₃ alkyl.

27. The compound of any one of claims 1-26, wherein R⁶ is H or C₁-C₃ alkyl.

28. The compound of any one of claims 1-27, wherein R⁸ is C(O)C₁-C₆ alkyl or NHC(O)C₁-C₆ alkyl.

29. The compound of any one of claims 1-28, wherein R² is selected from the group consisting of

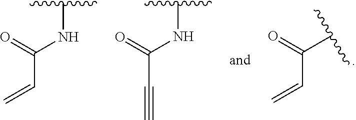

30. The compound of any one of claims 1, 6, and 10-28, where in R² is selected from the group consisting of

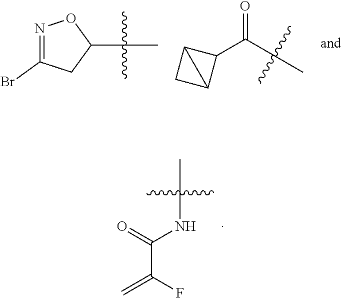

31. The compound of any one of claims 1, 6, 10-25, and 25, wherein R⁸ is selected from C₁-C₆ alkyl, C₃-C₁₀ cycloalkyl, 3-10 membered heterocycloalkyl, C₆-C₁₀ aryl, 5-10 membered heteroaryl, OH, CN, NH₂, C₁-C₆ alkyl-OH, 3-10 membered heterocycloalkyl, SO₂NH₂, SO₂NH(C₁-C₆ alkyl), SO₂NH(C₃-C₆ cycloalkyl), SO₂N(C₁-C₆ alkyl)₂, SO₂N(C₃-C₆ cycloalkyl)₂, NHSO₂ (C₁-C₆ alkyl), and NHSO₂ (C₃-C₁₀ cycloalkyl).

32. The compound of any one of claims 1, 6, 12-17, and 19-31, wherein each R⁹ is independently selected from halo, OH, C₁-C₆ alkyl, OC₁-C₆ alkoxy, NH₂, NH(C₁-C₆ alkyl), and N(C₁-C₆ alkyl)₂.

33. The compound of any one of claims 1-32, wherein the compound of Formula I is selected from the group consisting of 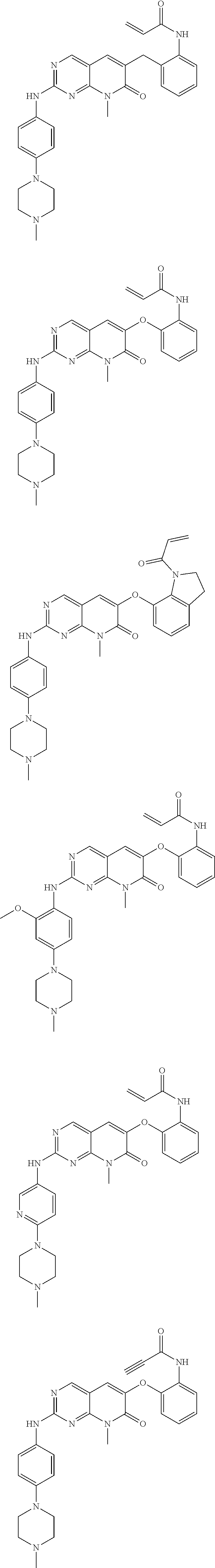 [SMILES: C=CC(=O)Nc1ccccc1Cc1cc2cnc(Nc3ccc(N4CCN(C)CC4)cc3)nc2n(C)c1=O ; C=CC(=O)Nc1ccccc1Oc1cc2cnc(Nc3ccc(N4CCN(C)CC4)cc3)nc2n(C)c1=O ; C=CC(=O)N1CCc2cccc(Oc3cc4cnc(Nc5ccc(N6CCN(C)CC6)cc5)nc4n(C)c3=O)c21 ; C=CC(=O)Nc1ccccc1Oc1cc2cnc(Nc3ccc(N4CCN(C)CC4)cc3OC)nc2n(C)c1=O ; C=CC(=O)Nc1ccccc1Oc1cc2cnc(Nc3ccc(N4CCN(C)CC4)nc3)nc2n(C)c1=O ; C#CC(=O)Nc1ccccc1Oc1cc2cnc(Nc3ccc(N4CCN(C)CC4)cc3)nc2n(C)c1=O] 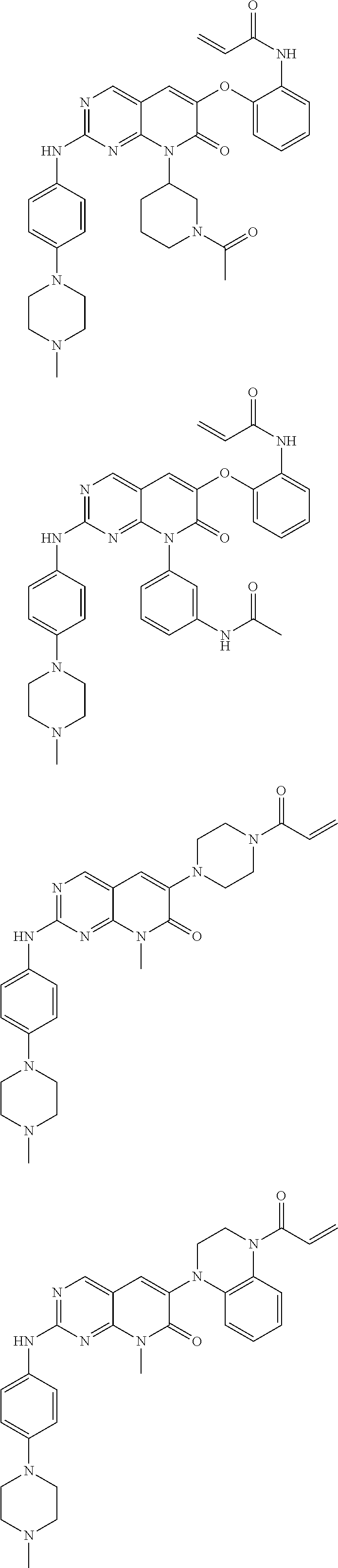 [SMILES: C=CC(=O)Nc1ccccc1Oc1cc2cnc(Nc3ccc(N4CCN(C)CC4)cc3)nc2n(C2CCCN(C(C)=O)C2)c1=O ; C=CC(=O)Nc1ccccc1Oc1cc2cnc(Nc3ccc(N4CCN(C)CC4)cc3)nc2n(-c2cccc(NC(C)=O)c2)c1=O ; C=CC(=O)N1CCN(c2cc3cnc(Nc4ccc(N5CCN(C)CC5)cc4)nc3n(C)c2=O)CC1 ; C=CC(=O)N1CCN(c2cc3cnc(Nc4ccc(N5CCN(C)CC5)cc4)nc3n(C)c2=O)c2ccccc21]
or a pharmaceutically acceptable salt thereof.

34. The compound of any one of claims 1-32, wherein the compound is selected from the group consisting of a compound in Table 2, or a pharmaceutically acceptable salt thereof.

35. A pharmaceutical composition comprising a compound of any one of claims 1-34, or a pharmaceutically acceptable salt thereof, and a pharmaceutically acceptable carrier.

36. A method of inhibiting the activity of EGFR in a subject in need thereof comprising administering to the subject a therapeutically effective amount of a compound of any one of claims 1-34 or the pharmaceutical composition of claim 35.

37. A method of treating cancer in a subject in need thereof comprising administering to the subject a therapeutically effective amount of a compound of any one of claims 1-34 or the pharmaceutical composition of claim 35.

38. The method of claim 37, wherein the cancer is selected from the group consisting of lung cancer, colon cancer, breast cancer, endometrial cancer, thyroid cancer, glioma, squamous cell carcinoma, and prostate cancer.

39. The method according to claim 37, wherein the cancer is non-small cell lung cancer (NSCLC).
